(19) United States
(12) Patent Application Publication
(10) Pub. No.: US 2023/0295119 A1
(43) Pub. Date: Sep. 21, 2023

(54) SUBSTITUTED PYRIDINES FOR THE TREATMENT OF INFLAMMATORY DISEASES

(71) Applicant: GOSSAMER BIO SERVICES, INC., San Diego, CA (US)

(72) Inventors: Craig Alan Coburn, San Diego, CA (US); Chad Alan Van Huis, San Diego, CA (US); Thomas Daniel Aicher, San Diego, CA (US)

(21) Appl. No.: 17/918,536

(22) PCT Filed: Apr. 14, 2021
(86) PCT No.: PCT/US2021/027329

§ 371 Date: Oct. 12, 2022

Related U.S. Application Data
(60) Provisional application No. 63/009,943, filed on Apr. 14, 2020.

(51) Int. Cl. C07D 401/14; A61P 29/00; A61K 45/06; C07D 401/12; C07D 213/75; C07D 405/12; C07D 413/14; C07D 491/107; C07D 491/08

(52) U.S. Cl. CPC C07D 401/14; A61K 45/06; A61P 29/00; C07D 213/75; C07D 401/12; C07D 405/12; C07D 413/14; C07D 491/08; C07D 491/107; C07B 2200/05

(57) ABSTRACT

Compounds having the structure of Formula (I) or pharmaceutically acceptable isomers, racemates, hydrates, solvates or salts thereof, where A, R¹, R^{2a}, R^{2b}, R^{2c} and R³ are as defined herein, are useful in the modulation of IL-12, IL-23 and/or IFNα by acting on TYK2 to cause signal transduction inhibition, as well as to pharmaceutical compositions containing the same and to methods of their use and preparation.

### FIELD OF THE INVENTION

The present invention generally relates to compounds useful in the modulation of IL-12, IL-23 and/or IFNα by acting on TYK2 to cause signal transduction inhibition, as well as to pharmaceutical compositions containing the same and to methods of their use and preparation.

### BACKGROUND

Janus kinases (or JAK’s) are an intracellular, non-receptor tyrosine kinase family consisting of four different subtypes, namely JAK1, JAK2, JAK3 and tyrosine kinase 2 (TYK2). JAK1, JAK2, and TYK2 are ubiquitously expressed, while JAK3 expression is limited to leukocytes. Cytokines mediate a broad range of biological functions and play pivotal roles in immunity and inflammation by regulating the survival, proliferation, differentiation and function of immune cells, as well as cells from other organ systems. JAKs bind to various cytokine receptors (interleukins, interferons and hemoproteins), leading to tyrosine phosphorylation and thereby activation of STAT (signal transducers and activators of transcription) proteins and ultimately transcriptional activation of specific genes. Thus, JAKs play a key role in modulating immune and inflammatory responses to a variety of cytokines.

The JAK proteins are relatively large (120-140 kDa), with defined structures featuring seven distinct regions named Janus Homology domains 1-7 (JH1-7). Cytokine receptors typically functional as heterodimers, and as a result, more than one type of JAK kinase is often associated with cytokine receptor complexes.

JAK1 associates with the type I interferon (e.g., IFNα), type II interferon (e.g., IFNγ), IL-2 and IL-6 cytokine receptor complexes. JAK1 knockout mice die perinatally from defects in LIP receptor signaling.

JAK2 associates with single-chain (e.g., EPO), IL-3 and interferon gamma cytokine receptor families. JAK2 knockout mice die of anemia and kinase activating mutations in JAK2 (e.g., JAK2 V617F) are associated with myeloproliferative disorders (MPDs). Complete JAK2 inhibition leads to thrombocytopenia.

JAK3 associates exclusively with the gamma common cytokine receptor chain, present in the IL-2, IL-4, IL-7, IL-9, IL-15 and IL-21 cytokine receptor complexes. JAK3 is critical for lymphoid cell development and proliferation. Mutations in JAK3 result in severe combined immunodeficiency (SCID). JAK3 and JAK3-mediated pathways have been targeted for immunosuppressive indications (e.g., transplantation rejection and rheumatoid arthritis).

TYK2 associates with the type I interferon (e.g., IFNα), IL-6, IL-10, IL-12 and IL-23 cytokine receptor complexes, particularly IL12, IL23 and IFNα. Primary cells derived from a TYK2 deficient human are defective in type I interferon, IL-6, IL-10, IL-12 and IL-23 signaling. TYK2 -/- mice are resistant to experimental arthritis, non-responsive to small amounts of IFN-α, and exhibit abnormal responses to inflammatory challenges. TYK2 plays an important role in immunity to infection, and autoimmune and inflammatory diseases. Further, TYK2 activating mutants and fusion proteins have been detected in patients with leukemic diseases suggesting TYK2 is a potent oncogene. Tumor immune surveillance is the immune system’s ability to identify and subsequently eliminate cancerous self, thus counteracting spontaneous cellular mutations that otherwise would have targeted proto-oncogenes or tumor suppressor genes. TYK2 is associated with tumor surveillance and carcinogenesis. (see Leitner et al, “Tyrosine kinase 2- surveillant of tumors and bona fide oncogene”, Cytokine 2017, 89, 209-218).

JAK’s as therapeutic targets have been explored and validated. Approved JAK inhibitor drugs include:
Ruxolitinib (Jakafi®) is a JAK1/2 dual inhibitor indicated for the treatment of polycythemia vera (PV), intermediate or high-risk myelofibrosis (MF), and steroid-refractory acute graft-versus-host disease (GVHD).
Baricitinib (Olumiant®) is a JAK1/2 dual inhibitor for the treatment of rheumatoid arthritis (RA), atopic dermatitis and systemic lupus erythematosus.
Tofacitinib (Xeljanz®) is a pan-JAK inhibitor for the treatment of moderate to severe rheumatoid arthritis (RA), psoriatic arthritis, and ulcerative colitis.
Ustekinumab (Stelara®) is a human IgG1κ monoclonal antibody targeting the p40 subunit of the IL-12 and IL-23 cytokines for treatment of moderate to severe active Crohn’s disease, moderate to severe active ulcerative colitis, moderate or severe psoriasis and active psoriatic arthritis.

Side effects of these drugs can be very serious and include infections (pneumonia, herpes zoster, UTI, tuberculosis, candidiasis, pneumocystosis, bacterial, viral and other infections), malignancy (lymphoma) and thrombosis (deep venous thrombosis (DVT) pulmonary embolism (PE), arterial thrombosis).

Studies have shown that inhibition of TYK2 can regulate interleukin-12 (IL12), interleukin-23 (IL23) and type I interferon (IFNα), while leaving other cytokines unaffected would minimize side effects. As such, selective inhibition of TYK2 is a potential therapeutic strategy for treatment of diseases related to regulation of IFNα, IL12, and IL23, while minimizing the side effects of other JAK family subtypes. Notably, no small molecule TYK2 inhibitor has been approved for therapeutic use. There is, therefore, a need for selective inhibitors of TYK2.

Described herein are compounds that modulate IL-12, IL-23 and/or IFNα by acting on TYK2, and methods for using them to treat diseases, conditions, syndromes, and the like, that are affected by 12, IL-23 and/or IFNα levels. Regulation of these factors may provide methods for re-balancing or regulating one or more biological pathways associated with abnormal conditions, particularly autoimmune disorders, such as but not limited to Psoriasis, Psoriatic Arthritis, Atopic Dermatitis, Crohn’s Disease, Ulcerative Colitis, Lupus Nephritis, Systemic lupus erythematosus (SLE), Alopecia Areata, Vitiligo and Hidradenitis Suppurativa. Also described herein are pharmaceutical compositions containing at least one compound according to the invention that are useful for the treatment of conditions related to the modulation of IL-12, IL-23 and/or IFNα. Also described are methods for the preparation of the compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1.

### BRIEF SUMMARY

In one embodiment, compounds are provided having the structure of Formula (I):

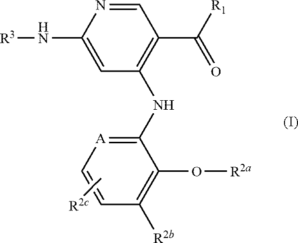

or a pharmaceutically acceptable salt, solvate, hydrate, isomer, tautomer, racemate, or isotope thereof, wherein:
A is N or CR^{2c};
R¹ is C₁₋₄ alkyl, C₃₋₆ cycloalkyl, C₁₋₄ haloalkyl, C₁₋₄ hydroxyalkyl or alkoxyalkyl;
R^{2a} is H, C₁₋₄ alkyl or C₁₋₄ fluoroalkyl;
R^{2b} is H, —CN, —C(O)OH, -C(O)OC₁₋₄alkyl, —C(O)NR⁵R⁶, or 5- or 6-membered heteroaryl, wherein R^{2b} is substituted with 0-2 R′;
R^{2c} is H, halo, —CN, C₁₋₄ alkyl, C₁₋₄ alkoxy or C₁₋₄haloalkyl;
R³ is H, C₂₋₄ alkoxy, —C(O)R⁷, carbocycle, heterocycle, aryl or heteroaryl, wherein R³ is substituted with 0-2 R′;
R⁵ is H or C₁₋₄ alkyl;
R⁶ is H, C₁₋₄ alkyl, -(CH₂)ₘ-carbocycle, -(CH₂)ₘ-heterocycle, -(CH₂)ₘ-aryl or -(CH₂)ₘ-heteroaryl;
R⁷ is C₁₋₄ alkyl, —(CH₂)ₙ—OH, -(CH₂)ₙ-OC₁₋₄alkyl, —(CH₂)ₙ—NH₂, -(CH₂)ₙ NHC₁₋₄alkyl, -(CH₂)ₙ-N(C₁₋₄alkyl)(C₁₋₄alkyl), -(CH₂)ₙ-carbocycle, -(CH₂)ₙ-heterocycle, -(CH₂)ₘ-aryl, -(CH₂)ₘ-heteroaryl or halocycloalkyl, wherein R⁷ is substituted with 0-2 R′;
R′ is —CN, —NO₂, halo, C₁₋₆ alkyl, C₁₋₆ haloalkyl, C₁₋₆ hydroxyalkyl, C₁₋₆ alkoxy, C₁₋₆ haloalkoxy, alkoxyalkyl, carbocycle, —(CH₂)_{q}—N(R)₂, —(CH₂)_{q}—C(O)R,
—(CH₂)_{q}—C(O)OR, —(CH₂)_{q}—C(O)N(R)₂, —(CH₂)_{q}—NHC(O)R, —(CH₂)_{q}—S(O)₂R, -(CH₂)_{q}-carbocycle or -(CH₂)_{q}-heterocycle;
each R is, independently, H, C₁₋₄ alkyl, carbocycle or heterocycle;
m is 0-2;
n is 0-2; and
q is 0-4.

In another embodiment, compounds are provided having the structure listed in Table 1, or a pharmaceutically acceptable isomer, racemate, hydrate, solvate, isotope, or salt thereof.

In another embodiment, a composition is provided, comprising a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or a pharmaceutically acceptable isomer, racemate, hydrate, solvate, isotope, or salt thereof, and a pharmaceutically acceptable carrier, diluent, or excipient.

In another embodiment, a use is provided for a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or a pharmaceutically acceptable salt, solvate, hydrate, isomer, tautomer, racemate, or isotope thereof in the manufacture of a medicament.

In another embodiment, a method for inhibiting tyrosine kinase 2 (TYK2) activity is provided, comprising contacting the TYK with an effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or a pharmaceutically acceptable salt, solvate, hydrate, isomer, tautomer, racemate, or isotope thereof

In another embodiment, a method for inhibiting tyrosine kinase 2 (TYK2) activity in a subject is provided, comprising administering to the subject an effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or a pharmaceutically acceptable salt, solvate, hydrate, isomer, tautomer, racemate, or isotope thereof.

In another embodiment, a method for modulating IL-12, IL-23 and/or IFNα is provided, comprising contacting the IL-12, IL-23 and/or IFNα with an effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or a pharmaceutically acceptable salt, solvate, hydrate, isomer, tautomer, racemate, or isotope thereof.

In another embodiment, a method is provided for treating a subject with Psoriasis, Psoriatic Arthritis, Atopic Dermatitis, Crohn’s Disease, Ulcerative Colitis, Lupus Nephritis, Systemic lupus erythematosus (SLE), Alopecia Areata, Vitiligo or Hidradenitis Suppurativa is provided, comprising administering to the subject an effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or a pharmaceutically acceptable salt, solvate, hydrate, isomer, tautomer, racemate, or isotope thereof.

In another embodiment, a method is provided for treating Psoriasis, Psoriatic Arthritis, Atopic Dermatitis, Crohn’s Disease, Ulcerative Colitis, Lupus Nephritis, Systemic lupus erythematosus (SLE), Alopecia Areata, Vitiligo or Hidradenitis Suppurativa, comprising administering to a subject an effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or a pharmaceutically acceptable salt, solvate, hydrate, isomer, tautomer, racemate, or isotope thereof.

### DETAILED DESCRIPTION

The present invention relates to pyridine compounds, pharmaceutical compositions containing them, methods of using them for the treatment of disease states, disorders and conditions related to the modulation of TYK2, IL-12, IL-23 and/or IFNα and methods for preparing them.

As used herein, “alkyl” means a straight chain or branched saturated hydrocarbon group. “Lower alkyl” means a straight chain or branched alkyl group having from 1 to 8 carbon atoms, in some embodiments from 1 to 6 carbon atoms, in some embodiments from 1 to 4 carbon atoms, and in some embodiments from 1 to 2 carbon atoms. Examples of straight chain lower alkyl groups include, but are not limited to, methyl, ethyl, n-propyl, n-butyl, n-pentyl, n-hexyl, n-heptyl, and n-octyl groups. Examples of branched lower alkyl groups include, but are not limited to, isopropyl, iso-butyl, sec-butyl, t-butyl, neopentyl, isopentyl, and 2,2-dimethylpropyl groups.

“Alkenyl” groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to —CH═CH2, —CH═CH(CH₃), —CH═C(CH₃)₂, —C(CH₃)═CH₂, —C(CH₃)═CH(CH₃), —C(CH₂CH₃)═CH₂, —CH═CHCH₂CH₃, —CH═CH(CH₂)₂CH₃, —CH═CH(CH₂)₃CH₃, —CH═CH(CH₂)₄CH₃, vinyl, cyclohexenyl, cyclopentenyl, cyclohexadienyl, butadienyl, pentadienyl, and hexadienyl among others.

“Alkynyl” groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others.

As used herein, “alkylene” means a divalent alkyl group. Examples of straight chain lower alkylene groups include, but are not limited to, methylene (i.e., —CH₂—), ethylene (i.e., —CH₂CH₂—), propylene (i.e., —CH₂CH₂CH₂—), and butylene (i.e., —CH₂CH₂CH₂CH₂—). As used herein, “heteroalkylene” is an alkylene group of which one or more carbon atoms is replaced with a heteroatom such as, but not limited to, N, O, S, or P.

“Alkoxy” refers to an alkyl as defined above joined by way of an oxygen atom (i.e., -O-alkyl). Examples of lower alkoxy groups include, but are not limited to, methoxy, ethoxy, n-propoxy, n-butoxy, isopropoxy, sec-butoxy, tert-butoxy, and the like.

The terms “carbocyclic” and “carbocycle” denote a ring structure wherein the atoms of the ring are carbon. Carbocycles may be monocyclic or polycyclic. Carbocycle encompasses both saturated and unsaturated rings. Carbocycle encompasses both cycloalkyl and aryl groups. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N substituents wherein N is the size of the carbocyclic ring with for example, alkyl, amino, hydroxy, cyano, carboxy, nitro, thio, alkoxy, and halogen groups.

“Cycloalkyl” groups are alkyl groups forming a ring structure, which can be substituted or unsubstituted. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups.

“Aryl” groups are cyclic aromatic hydrocarbons that do not contain heteroatoms. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include include fused rings wherein at least one ring, but not necessarily all rings, are aromatic, such as fused aromatic-aliphatic ring systems (e.g., indanyl, tetrahydronaphthyl, and the like).

“Carbocyclealkyl” refers to an alkyl as defined above with one or more hydrogen atoms replaced with carbocycle. Examples of carbocyclealkyl groups include but are not limited to benzyl and the like.

As used herein, “heterocycle” or “heterocyclyl” groups include aromatic and non-aromatic ring compounds (heterocyclic rings) containing 3 or more ring members, of which one or more is a heteroatom such as, but not limited to, N, O, S, or P. A heterocycle group as defined herein can be a heteroaryl group or a partially or completely saturated cyclic group including at least one ring heteroatom. In some embodiments, heterocycle groups include 3 to 20 ring members, whereas other such groups have 3 to 15 ring members. At least one ring contains a heteroatom, but every ring in a polycyclic system need not contain a heteroatom. For example, a dioxolanyl ring and a benzdioxolanyl ring system (methylenedioxyphenyl ring system) are both heterocycle groups within the meaning herein. A heterocycle group designated as a C2-heterocycle can be a 5-membered ring with two carbon atoms and three heteroatoms, a 6-membered ring with two carbon atoms and four heteroatoms and so forth. Likewise, a C4-heterocycle can be a 5-membered ring with one heteroatom, a 6-membered ring with two heteroatoms, and so forth. The number of carbon atoms plus the number of heteroatoms sums up to equal the total number of ring atoms. A saturated heterocyclic ring refers to a heterocyclic ring containing no unsaturated carbon atoms.

“Heteroaryl” groups are aromatic ring compounds containing 5 or more ring members, of which, one or more is a heteroatom such as, but not limited to, N, O, and S. A heteroaryl group designated as a C₂-heteroaryl can be a 5-membered ring with two carbon atoms and three heteroatoms, a 6-membered ring with two carbon atoms and four heteroatoms and so forth. Likewise, a C₄-heteroaryl can be a 5-membered ring with one heteroatom, a 6-membered ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl.

“Heterocyclealkyl” refers to an alkyl as defined above with one or more hydrogen atoms replaced with heterocycle. Examples of heterocyclealkyl groups include, but are not limited to, morpholinoethyl and the like.

“Halo” or “halogen” refers to fluorine, chlorine, bromine and iodine.

“Haloalkyl” refers to an alkyl as defined above with one or more hydrogen atoms replaced with halogen. Examples of lower haloalkyl groups include, but are not limited to, —CF₃, —CH₂CF₃, and the like.

“Haloalkoxy” refers to an alkoxy as defined above with one or more hydrogen atoms replaced with halogen. Examples of lower haloalkoxy groups include, but are not limited to -OCF₃, -OCH₂CF₃, and the like.

“Hydroxyalkyl” refers to an alkyl as defined above with one or more hydrogen atoms replaced with —OH. Examples of lower hydroxyalkyl groups include, but are not limited to —CH₂OH, —CH₂CH₂OH, and the like.

As used herein, the term “optionally substituted” refers to a group (e.g., an alkyl, carbocycle, or heterocycle) having 0, 1, or more substituents, such as 0-25, 0-20, 0-10 or 0-5 substituents. Substituents include, but are not limited to —OR^{x}, —NR^{x}R^{y}, —S(O)₂R^{x} or —S(O)₂OR^{x}, halogen, cyano, alkyl, haloalkyl, alkoxy, carbocycle, heterocycle, carbocyclalkyl, or heterocyclealkyl, wherein each R^{x} and R^{y} is, independently, H, alkyl, haloalkyl, carbocycle, or heterocycle, or R^{x} and R^{y}, together with the atom to which they are attached, form a 3-8 membered carbocycle or heterocycle.

Described herein are compounds having the structure of Formula (I):

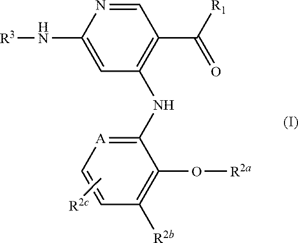

wherein:
A is N or CR^{2c};
R¹ is C₁₋₄ alkyl, C₃₋₆ cycloalkyl, C₁₋₄ haloalkyl, C₁₋₄ hydroxyalkyl or alkoxyalkyl;
R^{2a} is H, C₁₋₄ alkyl or C₁₋₄ fluoroalkyl;
R^{2b} is H, —CN, —C(O)OH, -C(O)OC₁₋₄alkyl, —C(O)NR⁵R⁶, or 5- or 6-membered heteroaryl, wherein R^{2b} is substituted with 0-2 R′;
R^{2c} is H, halo, —CN, C₁₋₄ alkyl, C₁₋₄ alkoxy or C₁₋₄haloalkyl;
R³ is H, C₂₋₄ alkoxy, —C(O)R⁷, carbocycle, heterocycle, aryl or heteroaryl, wherein R³ is substituted with 0-2 R′;
R⁵ is H or C₁₋₄ alkyl;
R⁶ is H, C₁₋₄ alkyl, -(CH₂)ₘ-carbocycle, -(CH₂)ₘ-heterocycle, -(CH₂)ₘ-aryl or -(CH₂)ₘ-heteroaryl;
R⁷ is C₁₋₄ alkyl, —(CH₂)ₙ—OH, -(CH₂)ₙ-OC₁₋₄alkyl, —(CH₂)ₙ—NH₂, -(CH₂)ₙ-NHC₁₋₄alkyl, -(CH₂)ₙ-N(C₁₋₄alkyl)(C₁₋₄alkyl), -(CH₂)ₙ-carbocycle, -(CH₂)ₙ-heterocycle, -(CH₂)ₘ-aryl, -(CH₂)ₘ-heteroaryl or halocycloalkyl, wherein R⁷ is substituted with 0-2 R′;
R′ is —CN, —NO₂, halo, C₁₋₆ alkyl, C₁₋₆ haloalkyl, C₁₋₆ hydroxyalkyl, C₁₋₆ alkoxy, _{C1-6} haloalkoxy, alkoxyalkyl, carbocycle, -(CH₂)_{q}—N(R)₂, —(CH₂)_{q}—C(O)R, —(CH₂)_{q}—C(O)OR, —(CH₂)_{q}—C(O)N(R)₂, —(CH₂)_{q}—NHC(O)R, —(CH₂)_{q}—S(O)₂R, -(CH₂)_{q}-carbocycle or -(CH₂)_{q}-heterocycle;
each R is, independently, H, C₁₋₄ alkyl, carbocycle or heterocycle;
m is 0-2;
n is 0-2; and
q is 0-4.

In some embodiments R^{2a} is H. In other embodiments R^{2a} is C₁₋₄ alkyl. In other embodiments R^{2a} is C₁₋₄ fluoroalkyl. In other embodiments R^{2a} is methyl. In other embodiments R^{2a} is ethyl. In other embodiments R^{2a} is difluoromethyl. In other embodiments R^{2a} is trifluoromethyl. In other embodiments R^{2a} is fluoroethyl.

In some embodiments R^{2b} is H. In other embodiments R^{2b} is —CN. In other embodiments R^{2b} is R^{2b} is —C(O)OH or -C(O)OC₁₋₄alkyl. In other embodiments R^{2b} is —C(O)OH. In other embodiments R^{2b} is -C(O)OC₁₋₄alkyl. In other embodiments R^{2b} is —C(O)OMe. In other embodiments R^{2b} is —C(O)NR⁵R⁶. In other embodiments R^{2b} is —C(O)NH₂. In other embodiments R^{2b} is -C(O)NHR⁶. In other embodiments R^{2b} is -C(O)NH-C₁₋₄ alkyl. In other embodiments R^{2b} is -C(O)NHMe. In other embodiments R^{2b} is —C(O)NMet. In other embodiments R^{2b} is -C(O)NHEt. In other embodiments R^{2b} is -C(O)NH-CH₂-carbocycle, -C(O)NH-CH₂-heterocycle, -C(O)NH-CH₂-aryl or -C(O)NH-CH₂-heteroaryl. In other embodiments R^{2b} is -C(O)NH-CH_{2\}-carbocycle, -C(O)NH-CH₂-heterocycle, -C(O)NH-CH₂-aryl or -C(O)NH-CH₂-heteroaryl, substituted with halo or C₁₋₆ alkyl. In other embodiments R^{2b} is -C(O)NH-CH₂-heterocycle or -C(O)NH-CH₂-heteroaryl, substituted with F or methyl. In other embodiments R^{2b} is -C(O)NH-(CH₂)ₘ-carbocycle. In other embodiments R^{2b} is -C(O)NH-carbocycle. In other embodiments R^{2b} is -C(O)NH-(CH₂)-carbocycle. In other embodiments R^{2b} is -C(O)NH-(CH₂)₂-carbocycle. In other embodiments R^{2b} is -C(O)NH-(CH₂)ₘ-heterocycle. In other embodiments R^{2b} is -C(O)NH-heterocycle. In other embodiments R^{2b} is -C(O)NH-(CH₂)-heterocycle. In other embodiments R^{2b} is -C(O)NH-(CH₂)₂-heterocycle. In other embodiments R^{2b} is -C(O)NH-(CH₂)ₘ-aryl. In other embodiments R^{2b} is -C(O)NH-aryl. In other embodiments R^{2b} is -C(O)NH-aryl substituted with 1 or 2 R′. In other embodiments R^{2b} is -C(O)NH-phenyl.

In other embodiments R^{2b} is -C(O)NH-phenyl substituted with 1 R′. In other embodiments R^{2b} is -C(O)NH-phenyl substituted with 2 R′. In other embodiments R^{2b} is -C(O)NH-(CH₂)-aryl. In other embodiments R^{2b} is -C(O)NH-(CH₂)-aryl substituted with 1 or 2 R′. In other embodiments R^{2b} is -C(O)NH-(CH₂)-aryl substituted with 1 R′. In other embodiments R^{2b} is -C(O)NH-(CH₂)-aryl substituted with 2 R′. In other embodiments R^{2b} is -C(O)NH-(CH₂)₂-aryl. In other embodiments R^{2b} is -C(O)NH-(CH₂)ₘ-heteroaryl. In other embodiments R^{2b} is -C(O)NH-(CH₂)ₘ-heteroaryl substituted with 1 or 2 R′. In other embodiments R^{2b} is -C(O)NH-(CH₂)ₘ-heteroaryl substituted with 1 R′. In other embodiments R^{2b} is -C(O)NH-(CH₂)ₘ-heteroaryl substituted with 2 R′. In other embodiments R^{2b} is -C(O)NH-heteroaryl. In other embodiments R^{2b} is -C(O)NH-(CH₂)-heteroaryl. In other embodiments R^{2b} is -C(O)NH-(CH₂)₂-heteroaryl.

In some embodiments R^{2b} is 5- or 6-membered heteroaryl, substituted with 0-2 R′. In other embodiments R^{2b} is a 5- or 6-membered heteroaryl. In other embodiments R^{2b} is a 5-membered heteroaryl. In other embodiments R^{2b} is a 6-membered heteroaryl. In other embodiments R^{2b} is a 5- or 6-membered heteroaryl substituted with 0-2 R′. In other embodiments R^{2b} is 5- or 6-membered heteroaryl, substituted with methyl. In other embodiments R^{2b} is 5membered heteroaryl, substituted with methyl. In other embodiments R^{2b} is 6-membered heteroaryl, substituted with methyl. In other embodiments R^{2b} is a 5-membered heteroaryl substituted with 0-2 R′. In other embodiments R^{2b} is a 6-membered heteroaryl substituted with 0-2 R′. In other embodiments R^{2b} is a. 5- or 6-membered heteroaryl substituted with 1 R′. In other embodiments R^{2b} is a. 5-membered heteroaryl substituted with 1 R′. In other embodiments R^{2b} is a. 6-membered heteroaryl substituted with 1 R′. In other embodiments R^{2b} is a. 5- or 6-membered heteroaryl substituted with 2 R′. In other embodiments R^{2b} is a. 5-membered heteroaryl substituted with 2 R′. In other embodiments R^{2b} is a. 6-membered heteroaryl substituted with 2 R′.

In some embodiments R^{2c} is H. In other embodiments R^{2c} is halo. In other embodiments R^{2c} is Cl. In other embodiments R^{2c} is F. In other embodiments R^{2c} is —CN. In other embodiments R^{2c} is C₁₋₄ alkyl. In other embodiments R^{2c} is Me. In other embodiments R^{2c} is Et.

In other embodiments R^{2c} is C₁₋₄ alkoxy. In other embodiments R^{2c} is OMe. In other embodiments R^{2c} is OEt. In other embodiments R^{2c} is C₁₋₄haloalkyl. In some embodiments R³ is H.In other embodiments R^{2c} is CF₃.

In some embodiments R³ is H. In other embodiments R³ is C₂₋₄ alkoxy. In other embodiments R³ is OMe. In other embodiments R³ isOEt. In other embodiments R³ is carbocycle, heterocycle, aryl or heteroaryl. In other embodiments R³ is carbocycle, heterocycle, aryl or heteroaryl, substituted with 1 or 2 R′. In other embodiments R³ is carbocycle, heterocycle, aryl or heteroaryl, substituted with 1 R′. In other embodiments R³ is carbocycle, heterocycle, aryl or heteroaryl, substituted with 2 R′. In other embodiments R³ is aryl or heteroaryl substituted with 1 or 2 R′. In other embodiments R³ is aryl or heteroaryl substituted with 1 R′. In other embodiments R³ is aryl or heteroaryl substituted with 2 R′. In other embodiments R³ is carbocycle. In other embodiments R³ is carbocycle, substituted with 1 or 2 R′. In other embodiments R³ is carbocycle, substituted with 1 R′. In other embodiments R³ is heterocycle. In other embodiments R³ is heterocycle, substituted with 1 or 2 R′. In other embodiments R³ is heterocycle, substituted with 1 R′. In other embodiments R³ is aryl. In other embodiments R³ is aryl, substituted with 1 or 2 R′. In other embodiments R³ is aryl, substituted with 1 R′. In other embodiments R³ is phenyl. In other embodiments R³ is phenyl, substituted with 1 or 2 R′. In other embodiments R³ is phenyl, substituted with 1 R′. In other embodiments R³ is heteroaryl. In other embodiments R³ is heteroaryl, substituted with 1 or 2 R′. In other embodiments R³ is heteroaryl, substituted with 1 R′. In other embodiments R³ is pyridyl. In other embodiments R³ is pyridyl, substituted with 1 or 2 R′. In other embodiments R³ is pyridyl, substituted with 1 R′.

In some embodiments R³ is —C(O)R⁷. In some embodiments R⁷ is -(CH₂)ₙ-carbocycle, -(CH₂)ₙ-heterocycle, -(CH₂)ₘ-aryl or -(CH₂)ₘ-heteroaryl. In some embodiments R⁷ is -(CH₂)ₙ-carbocycle, -(CH₂)ₙ-heterocycle, -(CH₂)ₘ-aryl or -(CH₂)ₘ-heteroaryl, substituted with 1 or 2 R′. In some embodiments R⁷ is -(CH₂)ₙ-carbocycle, -(CH₂)ₙ-heterocycle, -(CH₂)ₘ-aryl or -(CH₂)ₘ-heteroaryl, substituted with 1 R′. In other embodiments R⁷ is C₁₋₄ alkyl or carbocycle. In other embodiments R⁷ is C₁₋₄ alkyl or carbocycle, substituted with 1 or 2 R′. In other embodiments R⁷ is C₁₋₄ alkyl or carbocycle, substituted with 1 R′. In other embodiments R⁷ is carbocycle, heterocycle, aryl or heteroaryl. In other embodiments R⁷ is carbocycle, heterocycle, aryl or heteroaryl, substituted with 1 or 2 R′. In other embodiments R⁷ is carbocycle, heterocycle, aryl or heteroaryl, substituted with 1 R′.

In other embodiments R³ is -C(O) C₁₋₄ alkyl. In other embodiments R³ is -C(O)Me.

In other embodiments R³ is —C(O)Et. In other embodiments R³ is -C(O)-halocycloalkyl. In other embodiments R³ is —C(O)—(CH₂)ₙ—OH. In other embodiments R³ is —C(O)OH. In other embodiments R³ is —C(O)—(CH₂)—OH. In other embodiments R³ is —C(O)—(CH₂)₂—OH.

In other embodiments R³ is -C(O)-(CH₂)ₙ-OC₁₋₄alkyl. In other embodiments R³ is -C(O)-OC₁₋₄alkyl. In other embodiments R³ is -C(O)-(CH₂)-OC₁₋₄alkyl. In other embodiments R³ is -C(O)-(CH₂)₂-OC₁₋₄alkyl. In other embodiments R³ is —C(O)—(CH₂)ₙ—OMe. In other embodiments R³ is —C(O)—OMe. In other embodiments R³ is —C(O)—(CH₂)—OMe. In other embodiments R³ is —C(O)—(CH₂)₂—OMe. In other embodiments R³ is —C(O)—(CH₂)ₙ—NH₂. In other embodiments R³ is —C(O)—NH₂. In other embodiments R³ is —C(O)—(CH₂)—NH₂. In other embodiments R³ is —C(O)—(CH₂)₂—NH₂. In other embodiments R³ is -C(O)-(CH₂)ₙ-NHC₁₋₄alkyl. In other embodiments R³ is -C(O)-NHC₁₋₄alkyl. In other embodiments R³ is -C(O)-(CH₂)-NHC₁₋₄alkyl. In other embodiments R³ is -C(O)-(CH₂)₂-NHC₁₋₄alkyl. In other embodiments R³ is -C(O)-(CH₂)ₙ-NHMe. In other embodiments R³ is -C(O)-NHMe. In other embodiments R³ is -C(O)-(CH₂)-NHMe. In other embodiments R³ is -C(O)-(CH₂)₂-NHMe. In other embodiments R³ is -C(O)-(CH₂)ₙ-N(C₁₋₄alkyl)(C₁₋₄alkyl). In other embodiments R³ is -C(O)-N(C₁₋₄alkyl)(C₁₋₄alkyl). In other embodiments R³ is -C(O)-(CH₂)-N(C₁₋₄alkyl)(C₁₋₄alkyl). In other embodiments R³ is -C(O)-(CH₂)₂-N(C₁₋₄alkyl)(C₁₋₄alkyl). In other embodiments R³ is —C(O)—(CH₂)ₙ—NMe₂. In other embodiments R³ is —C(O)—NMe₂. In other embodiments R³ is —C(O)—(CH₂)—NMe₂. In other embodiments R³ is —C(O)—(CH₂)₂—NMe₂. In other embodiments R³ is —C(O)—(CH₂)ₙ—carbocycle. In other embodiments R³ is -C(O)-carbocycle. In other embodiments R³ is -C(O)-(CH₂)-carbocycle. In other embodiments R³ is -C(O)-(CH₂2-carbocycle. In other embodiments R³ is -C(O)-(CH₂)ₙ-heterocycle. In other embodiments R³ is -C(O)-heterocycle. In other embodiments R³ is -C(O)-(CH₂)-heterocycle. In other embodiments R³ is -C(O)-(CH₂)₂-heterocycle. In other embodiments R³ is -C(O)-(CH₂)ₘ-aryl. In other embodiments R³ is -C(O)-aryl. In other embodiments R³ is -C(O)-(CH₂)-aryl. In other embodiments R³ is -C(O)-(CH₂)₂-aryl. In other embodiments R³ is -C(O)-(CH₂)ₘ-heteroaryl. In other embodiments R³ is -C(O)-heteroaryl. In other embodiments R³ is -C(O)-(CH₂)-heteroaryl. In other embodiments R³ is -C(O)-(CH₂)₂-heteroaryl.

In other embodiments R⁷ is substituted with 1 or 2 R′. In other embodiments R⁷ is unsubstituted. In other embodiments R⁷ is substituted with 1 R′. In other embodiments R⁷ is substituted with 2 R′. In other embodiments m is 0, 1 or 2. In other embodiments m is 0. In other embodiments m is 1. In other embodiments m is 2. In other embodiments n is 0, 1 or 2. In other embodiments n is 0. In other embodiments n is 1. In other embodiments n is 2.

In some embodiments R′ is halo. In other embodiments R′ is Cl. In other embodiments R′ is F. In other embodiments R′ is C₁₋₆ alkyl. In other embodiments R′ is Me. In other embodiments R′ is Et. In other embodiments R′ is C₁₋₆ alkoxy. In other embodiments R′ is —OMe. In other embodiments R′ is —OEt. In other embodiments R′ is -CN. In other embodiments R′ is —NO₂. In other embodiments R′ is C₁₋₆ haloalkyl. In other embodiments R′ is CF₃. In other embodiments R′ is C₁₋₆ hydroxyalkyl. In other embodiments R′ is CH₂OH. In other embodiments R′ is C₁₋₆ alkoxy. In other embodiments R′ is OMe. In other embodiments R′ is C₁₋₆ haloalkoxy. In other embodiments R′ is OCF₃. In other embodiments R′ is alkoxyalkyl. In other embodiments R′ is CH₂OMe. In other embodiments R′ is a carbocycle. In other embodiments R′ is a heterocycle. In other embodiments R′ is —(CH₂)_{q}—N(R)₂. In other embodiments R′ is —(CH₂)_{q}—NH_{2.} In other embodiments R′ is —(CH₂)_{q}—NHMe. In other embodiments R′ is —(CH₂)_{q}—NMe_{2.} In other embodiments R′ is -(CH₂)_{q}-NH-carbocycle. In other embodiments R′ is -(CH₂)_{q}-NH- heterocycle.

In other embodiments R′ is —N(R)₂. In other embodiments R′ is —NH₂. In other embodiments R′ is —NMe₂. In other embodiments R′ is —NHMe. In other embodiments R′ is -NH-carbocycle. In other embodiments R′ is -NH-heterocycle. In other embodiments R′ is —(CH_{2)q}—C(O)R. In other embodiments R′ is —(CH₂)_{q}—C(O)H. In other embodiments R′ is —(CH₂)_{q}—C(O)Me. In other embodiments R′ is -(CH₂)_{q}-C(O)-carbocycle. In other embodiments R′ is -(CH₂)_{q}-C(O)-heterocycle. In other embodiments R′ is -C(O)R. In other embodiments R′ is —C(O)H. In other embodiments R′ is —C(O)Me. In other embodiments R′ is -C(O)-carbocycle. In other embodiments R′ is -C(O)-heterocycle. In other embodiments R′ is —(CH₂)_{q}—C(O)OR. In other embodiments R′ is —(CH₂)_{q}—C(O)OH. In other embodiments R′ is —(CH₂)_{q}—C(O)OMe. In other embodiments R′ is -(CH₂)_{q}-C(O)O-carbocycle. In other embodiments R′ is -(CH₂)_{q}-C(O)O-heterocycle. In other embodiments R′ is —C(O)OR. In other embodiments R′ is —C(O)OH. In other embodiments R′ is —C(O)OMe. In other embodiments R′ is -C(O)O-carbocycle. In other embodiments R′ is -C(O)O-heterocycle. In other embodiments R′ is —(CH₂)_{q}—C(O)N(R)₂. In other embodiments R′ is —(CH₂)_{q}—C(O)NH₂. In other embodiments R′ is —(CH₂)_{q}—C(O)NMe_{2.} In other embodiments R′ is -(CH₂)_{q} C(O)NHMe. In other embodiments R′ is -(CH₂)_{q}-C(O)NH-carbocycle. In other embodiments R′ is -(CH₂)_{q}-C(O)NH-heterocycle. In other embodiments R′ is —C(O)N(R)₂. In other embodiments R′ is —C(O)NH₂. In other embodiments R′ is —C(O)NMe₂. In other embodiments R′ is -C(O)NHMe. In other embodiments R′ is —C(O)NH—carbocycle. In other embodiments R′ is -C(O)NH-heterocycle. In other embodiments R′ is —(CH₂)_{q}—NHC(O)R. In other embodiments R′ is —(CH₂)_{q}—NHC(O)H. In other embodiments R′ is -(CH₂)_{q}-NHC(O)Me.

In other embodiments R′ is -NHC(O)R. In other embodiments R′ is -NHC(O)H. In other embodiments R′ is -NHC(O)Me. In other embodiments R′ is —(CH₂)_{q}—S(O)₂R. In other embodiments R′ is —(CH₂)_{q}—S(O)₂H. In other embodiments R′ is —(CH₂)_{q}—S(O)₂Me.

In other embodiments R′ is —S(O)₂R. In other embodiments R′ is —S(O)₂H. In other embodiments R′ is —S(O)₂Me.

In some embodiments R′ is a carbocycle. In other embodiments R′ is -(CH₂)_{q}-heterocycle. In other embodiments R′ is a heterocycle. In other embodiments R′ is -(CH₂)-heterocycle. In other embodiments R′ is -(CH₂)₂-heterocycle.

In one embodiment, R′ is R⁸ or R⁹, as defined below. That is, R⁸ and R⁹ are embodiments of R′.

In one embodiment, a compound is provided having the structure of Formula (II):

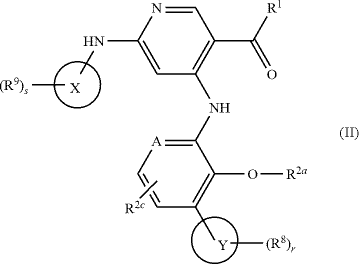

or a pharmaceutically acceptable isomer, racemate, hydrate, solvate, or salt thereof, wherein:
A is N or CR^{2c};
ring X is a 5- or 6-membered heteroaryl;
ring Y is heteroaryl;
R¹ is C₁₋₄ alkyl, C₃₋₆ cycloalkyl, C₁₋₄ haloalkyl, C₁₋₄ hydroxyalkyl or alkoxyalkyl;
R^{2a} is H, C₁₋₄ alkyl or C₁₋₄ fluoroalkyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, C₁₋₆ haloalkyl, -OR^{11a}, -NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, C₁₋₆ haloalkyl, carbocycle, heterocycle, —CH₂CN, —OH, or —C(O)OH; and
   R^{11b} is, at each occurrence, independently, H, halo, C₁₋₆ alkyl, C₁₋₆ haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle;
q is 0-4;
r is 0-2; and
s is 0-2.

In one embodiment, a compound having the structure of Formula (II) is provided, or a pharmaceutically acceptable salt, solvate, hydrate, isomer, tautomer, racemate, or isotope thereof, wherein ring Y is a 5- or 6-membered heteroaryl. In one embodiment, ring Y is a 5-membered heteroaryl. In one embodiment, ring Y is pyrrolyl, pyrazolyl, imidazolyl, triazolyl, furanyl, oxazolyl, oxadiazolyl, thiophenyl, thiazolyl, or thiadiazolyl. In another embodiment, ring Y is triazolyl. In another embodiment, ring Y is a 6-membered heteroaryl. In one embodiment, ring Y is pyridinyl, pyrimidinyl, or pyridazinyl.

In one embodiment, a compound having the structure of Formula (II) is provided, or a pharmaceutically acceptable salt, solvate, hydrate, isomer, tautomer, racemate, or isotope thereof, wherein R¹ is C₁₋₄ alkyl. In one embodiment, R¹ is methyl. In another embodiment, R¹ is ethyl. In another embodiment, R¹ is propyl or butyl. In one embodiment, R¹ is C₃₋₆ cycloalkyl. In one embodiment, R¹ is cyclopropyl. In another embodiment, R¹ is cyclobutyl. In another embodiment, R¹ is cyclopentyl. In another embodiment, R¹ is cyclohexyl. In another embodiment, R¹ is C₁₋₄ hydroxyalkyl. In another embodiment, R¹ is alkoxyalkyl.

In another embodiment, a compound having the structure of Formula (II) is provided, or a pharmaceutically acceptable salt, solvate, hydrate, isomer, tautomer, racemate, or isotope thereof, wherein R^{2a} is H. In another embodiment, R^{2a} is C₁₋₄ alkyl. In another embodiment, R^{2a} is C₁₋₄ fluoroalkyl. In a further embodiment, R^{2a} is methyl. In another embodiment, R^{2a} is ethyl. In one embodiment, R^{2a} is difluoromethyl. In another embodiment, R^{2a} is trifluoromethyl. In another embodiment, R^{2a} is fluoroethyl.

In another embodiment, a compound having the structure of Formula (II) is provided, or a pharmaceutically acceptable salt, solvate, hydrate, isomer, tautomer, racemate, or isotope thereof, wherein r is 0. In another embodiment, r is 1. In another embodiment, r is 2.

In one embodiment, a compound is provided having the structure of Formula (III):

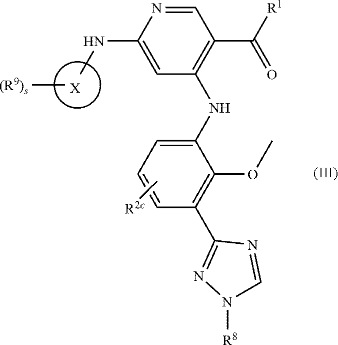

or a pharmaceutically acceptable salt, solvate, hydrate, isomer, tautomer, racemate, or isotope thereof, wherein:
ring X is a 5- or 6-membered heteroaryl;
R¹ is ethyl or cyclopropyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, C₁₋₆ haloalkyl, -OR^{11a}, -NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1-6} haloalkyl, carbocycle, heterocycle, —CH₂CN, —OH, or —C(O)OH; and
   R^{11b} is, at each occurrence, independently, H, halo, C₁₋₆ alkyl, _{C1-6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle;
q is 0-4;
r is 0-2; and
s is 0-2.

In one embodiment, a compound is provided having the structure of Formula (III-i):

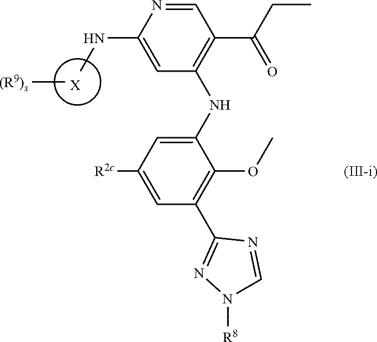

or a pharmaceutically acceptable salt, solvate, hydrate, isomer, tautomer, racemate, or isotope thereof, wherein:
ring X is a 5- or 6-membered heteroaryl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1- 6} haloalkyl, —OR^{11a}, —NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, C₁₋₆ haloalkyl, carbocycle, heterocycle, —CH₂CN, —OH, or —C(O)OH; and
   R^{11b} is, at each occurrence, independently, H, halo, C₁₋₆ alkyl, C₁₋₆ haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle;
q is 0-4;
r is 0-2; and
s is 0-2.

In one embodiment, a compound is provided having the structure of Formula (III-ii):

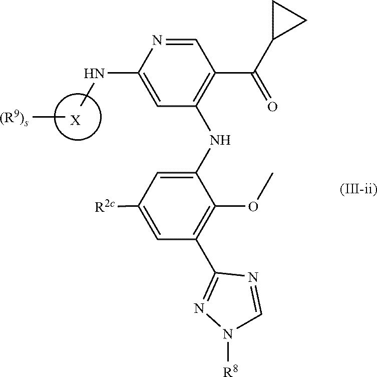

or a pharmaceutically acceptable salt, solvate, hydrate, isomer, tautomer, racemate, or isotope thereof, wherein:
ring X is a 5- or 6-membered heteroaryl;
R¹ is ethyl or cyclopropyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1- 6} haloalkyl, —OR^{11a}, —NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1- 6} haloalkyl, carbocycle, heterocycle, —CH₂CN, —OH, or —C(O)OH; and
   R^{11b} is, at each occurrence, independently, H, halo, C₁₋₆ alkyl, _{C1- 6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle;
q is 0-4;
r is 0-2; and
s is 0-2.

In one embodiment, a compound having the structure of any one of Formula (II), (III), (III-i), or (III-ii) is provided, or a pharmaceutically acceptable salt, solvate, hydrate, isomer, tautomer, racemate, or isotope thereof, wherein ring X is a 5-membered heteroaryl. In another embodiment, ring X is pyrrolyl, pyrazolyl, imidazolyl, triazolyl, furanyl, oxazolyl, oxadiazolyl, thiophenyl, thiazolyl, or thiadiazolyl. In another embodiment, ring X is pyrazolyl or imidazolyl. In another embodiment, ring X is pyrazolyl. In another embodiment, ring X is a 6-membered heteroaryl. In one embodiment, ring X is pyridinyl, pyrimidinyl, or pyridazinyl.

In one embodiment, a compound is provided having the structure of Formula (IV):

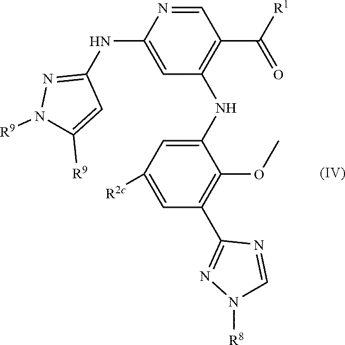

or a pharmaceutically acceptable salt, solvate, hydrate, isomer, tautomer, racemate, or isotope thereof, wherein:
R¹ is ethyl or cyclopropyl;
R^{2c} is H, halo, —CN, C₁₋₄ alkyl, C₁₋₄ alkoxy, or C₁₋₄haloalkyl;
R⁸ is C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1- 6} haloalkyl, -OR^{11a}, -NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1- 6} haloalkyl, carbocycle, heterocycle, —CH₂CN, —OH, or —C(O)OH; and
   R^{11b} is, at each occurrence, independently, H, halo, C₁₋₆ alkyl, _{C1- 6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4.

In another embodiment, a compound is provided having the structure of Formula (IV-i):

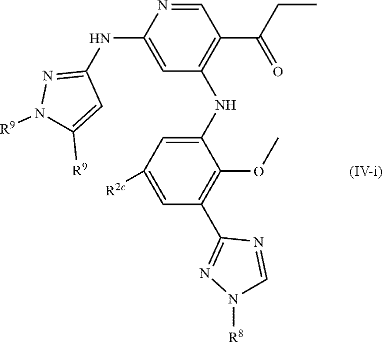

or a pharmaceutically acceptable salt, solvate, hydrate, isomer, tautomer, racemate, or isotope thereof, wherein:
R^{2c} is H, halo, —CN, C₁₋₄ alkyl, C₁₋₄ alkoxy, or C₁₋₄haloalkyl;
R⁸ is C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1- 6} haloalkyl, —OR^{11a}, —NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1- 6} haloalkyl, carbocycle, heterocycle, —CH₂CN, —OH, or —C(O)OH; and
   R^{11b} is, at each occurrence, independently, H, halo, C₁₋₆ alkyl, _{C1- 6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4.

In another embodiment, a compound is provided having the structure of Formula (IV-ii):

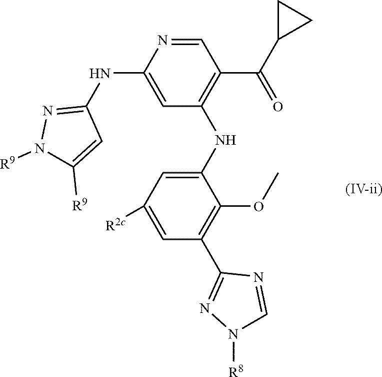

or a pharmaceutically acceptable salt, solvate, hydrate, isomer, tautomer, racemate, or isotope thereof, wherein:
R^{2c} is H, halo, —CN, C₁₋₄ alkyl, C₁₋₄ alkoxy, or C₁₋₄haloalkyl;
R⁸ is C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1- 6} haloalkyl, —OR^{11a}, —NR^{11a}R^{11b}, —SO₂R^{11a}, —SO₂NR^{11a}R^{11b}, —SO(═NH)R^{11a},
—C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1- 6} haloalkyl, carbocycle, heterocycle, —CH₂CN, —OH, or —C(O)OH; and
   R^{11b} is, at each occurrence, independently, H, halo, C₁₋₆ alkyl, _{C1- 6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4.

In one embodiment, a compound having the structure of any one of Formula (II), (III), (III-i), (III-ii), (IV), (IV-i), or (IV-ii) is provided, or a pharmaceutically acceptable salt, solvate, hydrate, isomer, tautomer, racemate, or isotope thereof, wherein R^{2c} is H. In other embodiments R^{2c} is halo. In other embodiments R^{2c} is Cl. In other embodiments, R^{2c} is F. In other embodiments R^{2c} is -CN. In other embodiments R^{2c} is C₁₄ alkyl. In other embodiments R^{2c} is methyl. In other embodiments R^{2c} is ethyl. In other embodiments R^{2c} is C₁₋₄ alkoxy. In other embodiments, R^{2c} is -OCH₃. In other embodiments, R^{2c} is -OCH₂CH₃. In other embodiments, R^{2c} is C₁₋₄ haloalkyl. In other embodiments, R^{2c} is —CF₃.

In one embodiment, a compound having the structure of any one of Formula (II), (III), (III-i), (III-ii), (IV), (IV-i), or (IV-ii) is provided, or a pharmaceutically acceptable salt, solvate, hydrate, isomer, tautomer, racemate, or isotope thereof, wherein R⁸ is C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle. In one embodiment, R⁸ is C₁₋₆ alkyl. In one embodiment, R⁸ is methyl. In another embodiment, R⁸ is alkoxyalkyl.

In one embodiment, a compound is provided having the structure of Formula (V):

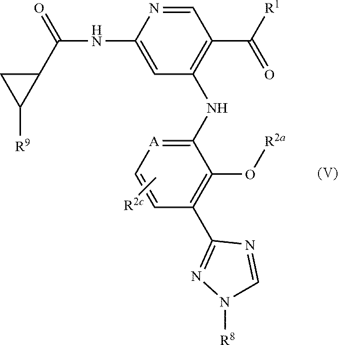

or a pharmaceutically acceptable salt, solvate, hydrate, isomer, tautomer, racemate, or isotope thereof, wherein:
A is N or CR^{2c};
R¹ is C₁₋₄ alkyl, C₃₋₆ cycloalkyl, C₁₋₄ haloalkyl, C₁₋₄ hydroxyalkyl or alkoxyalkyl;
R^{2a} is H, C₁₋₄ alkyl or C₁₋₄ fluoroalkyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1- 6} haloalkyl, —OR^{11a}, —NR^{11a}R^{11b}, —SO₂R^{11a}, -SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1- 6} haloalkyl, carbocycle, heterocycle, —CH₂CN, —OH, or —C(O)OH; and
   R^{11b} is, at each occurrence, independently, H, halo, C₁₋₆ alkyl, _{C1- 6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4;
wherein R^{2c} is halo, —CN, C₁₋₄ alkyl, C₁₋₄ alkoxy, or C₁₋₄haloalkyl when R⁹ is H.

In one embodiment, a compound is provided having the structure of Formula (VI):

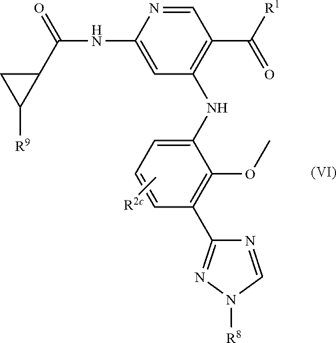

or a pharmaceutically acceptable salt, solvate, hydrate, isomer, tautomer, racemate, or isotope thereof, wherein:
R¹ is C₁₋₄ alkyl or C₃₋₆ cycloalkyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1-6} haloalkyl, -OR^{11a}, -NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1- 6} haloalkyl, carbocycle, heterocycle, —CH₂CN, —OH, or —C(O)OH; and
   R^{11b} is, at each occurrence, independently, H, halo, C₁₋₆ alkyl, _{C1- 6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4;
wherein R^{2c} is halo, —CN, C₁₋₄ alkyl, C₁₋₄ alkoxy, or C₁₋₄haloalkyl when R⁹ is H.

In one embodiment, a compound is provided having the structure of Formula (VI-A):

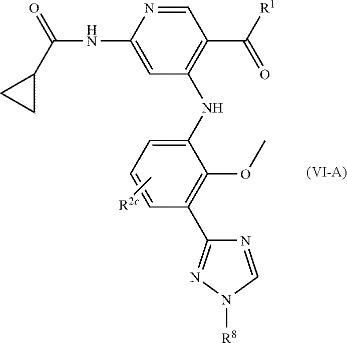

or a pharmaceutically acceptable salt, solvate, hydrate, isomer, tautomer, racemate, or isotope thereof, wherein:
R¹ is C₁₋₄ alkyl or C₃₋₆ cycloalkyl;
R^{2c} is halo, —CN, C₁₋₄ alkyl, C₁₋₄ alkoxy, or C₁₋₄haloalkyl; and
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle.

In one embodiment, a compound is provided having the structure of Formula (VI-A-i):

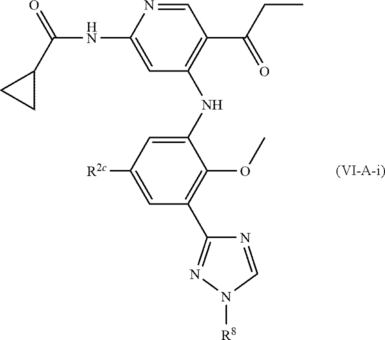

or a pharmaceutically acceptable salt, solvate, hydrate, isomer, tautomer, racemate, or isotope thereof, wherein:
R^{2c} is halo, —CN, C₁₋₄ alkyl, C₁₋₄ alkoxy, or C₁₋₄haloalkyl; and
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle.

In one embodiment, a compound is provided having the structure of Formula (VI-A-ii):

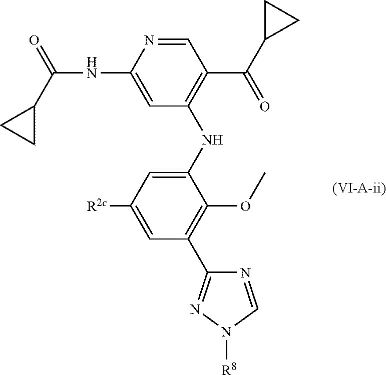

or a pharmaceutically acceptable salt, solvate, hydrate, isomer, tautomer, racemate, or isotope thereof, wherein:
R^{2c} is halo, —CN, C₁₋₄ alkyl, C₁₋₄ alkoxy, or C₁₋₄haloalkyl; and
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle.

In one embodiment, a compound having the structure of any one of Formula (V), (VI), (VI-A), (VI-A-i), or (VI-A-ii),is provided, or a pharmaceutically acceptable salt, solvate, hydrate, isomer, tautomer, racemate, or isotope thereof, wherein R^{2c} is R^{2c} is halo. In other embodiments R^{2c} is Cl. In other embodiments, R^{2c} is F. In other embodiments R^{2c} is —CN. In other embodiments R^{2c} is C₁₄ alkyl. In other embodiments R^{2c} is methyl. In other embodiments R^{2c} is ethyl. In other embodiments R^{2c} is C₁₋₄ alkoxy. In other embodiments, R^{2c} is -OCH₃. In other embodiments, R^{2c} is -OCH₂CH₃. In other embodiments, R^{2c} is C₁₋₄ haloalkyl. In other embodiments, R^{2c} is -CF₃.

In one embodiment, a compound is provided having the structure of Formula (VI-B):

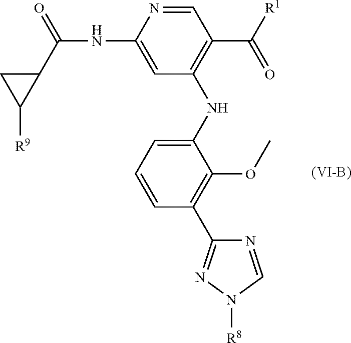

or a pharmaceutically acceptable salt, solvate, hydrate, isomer, tautomer, racemate, or isotope thereof, wherein:
R¹ is C₁₋₄ alkyl or C₃₋₆ cycloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1- 6} haloalkyl, —OR^{11a}, —NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1- 6} haloalkyl, carbocycle, heterocycle, —CH₂CN, —OH, or —C(O)OH; and
   R^{11b} is, at each occurrence, independently, H, halo, C₁₋₆ alkyl, _{C1- 6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4.

In one embodiment, a compound is provided having the structure of Formula (VI-B-i):

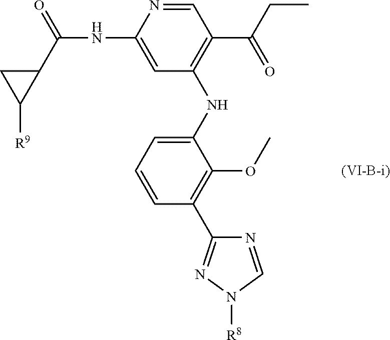

or a pharmaceutically acceptable salt, solvate, hydrate, isomer, tautomer, racemate, or isotope thereof, wherein:
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1-6} haloalkyl, —OR^{11a}, —NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1-6} haloalkyl, carbocycle, heterocycle, —CH₂CN, —OH, or —C(O)OH; and
   R^{11b} is, at each occurrence, independently, H, halo, C₁₋₆ alkyl, _{C1-6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4.

In one embodiment, a compound is provided having the structure of Formula (VI-B-ii):

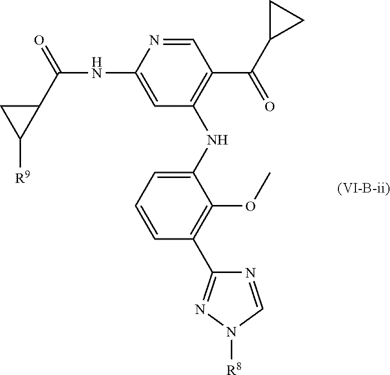

or a pharmaceutically acceptable salt, solvate, hydrate, isomer, tautomer, racemate, or isotope thereof, wherein:
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, _{C1- 6} haloalkyl, —OR^{11a}, —NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
   R^{11a} is, at each occurrence, independently, H, halo, C₁₋₆ alkyl, _{C1- 6} haloalkyl, carbocycle, heterocycle, —CH₂CN, —OH, or —C(O)OH; and
   R^{11b} is, at each occurrence, independently, H, halo, C₁₋₆ alkyl, _{C1- 6} haloalkyl, carbocycle, heterocycle, —CH₂CN, —OH, or —C(O)OH; or
   R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4.

In one embodiment, a compound having the structure of any one of Formula (V), (VI), (VI-A), (VI-A-i), (VI-A-ii), (VI-B), (VI-B-i), or (VI-B-ii) is provided, or a pharmaceutically acceptable salt, solvate, hydrate, isomer, tautomer, racemate, or isotope thereof, wherein R⁸ is C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle. In one embodiment, R⁸ is C₁₋₆ alkyl. In one embodiment, R⁸ is methyl. In another embodiment, R⁸ is alkoxyalkyl.

In one embodiment, a compound is provided, or a pharmaceutically acceptable salt, solvate, hydrate, isomer, tautomer, racemate, or isotope thereof, having the structure of a compound listed in Table 1, below:

TABLE 1

REPRESENTATIVE COMPOUNDS

| | |
|---|---|
| Compound No. | Structure |
| 1A | 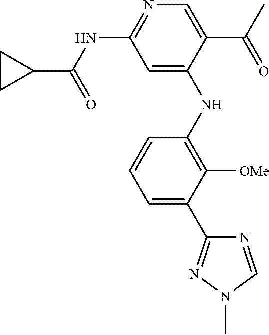 [SMILES: COc1c(Nc2cc(NC(=O)C3CC3)ncc2C(C)=O)cccc1-c1ncn(C)n1] |
| 1B | 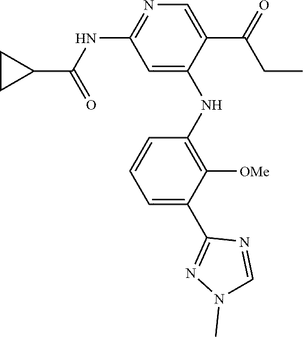 [SMILES: CCC(=O)c1cnc(NC(=O)C2CC2)cc1Nc1cccc(-c2ncn(C)n2)c1OC] |
| 1C | 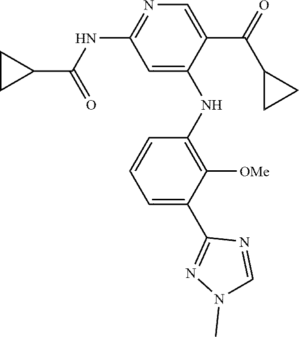 [SMILES: COc1c(Nc2cc(NC(=O)C3CC3)ncc2C(=O)C2CC2)cccc1-c1ncn(C)n1] |
| 1D | 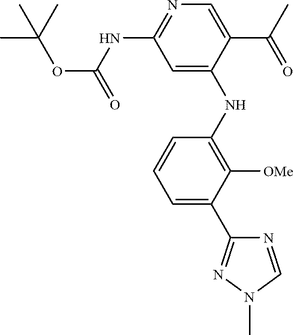 [SMILES: COc1c(Nc2cc(NC(=O)OC(C)(C)C)ncc2C(C)=O)cccc1-c1ncn(C)n1] |
| 1G | 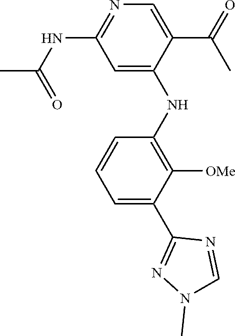 [SMILES: COc1c(Nc2cc(NC(C)=O)ncc2C(C)=O)cccc1-c1ncn(C)n1] |
| 1H | 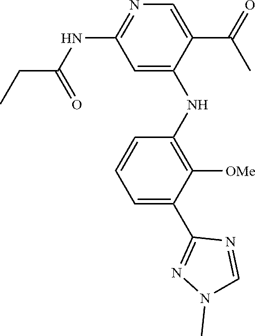 [SMILES: CCC(=O)Nc1cc(Nc2cccc(-c3ncn(C)n3)c2OC)c(C(C)=O)cn1] |
| 1I | 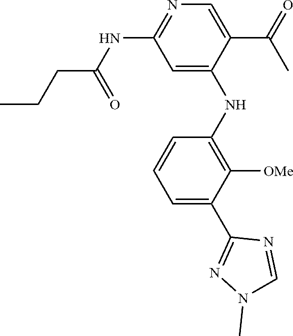 [SMILES: CCCC(=O)Nc1cc(Nc2cccc(-c3ncn(C)n3)c2OC)c(C(C)=O)cn1] |
| 1J | 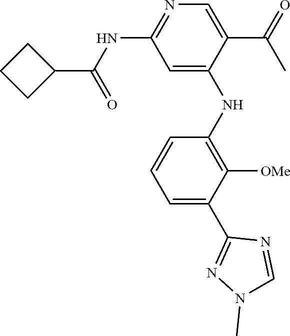 [SMILES: COc1c(Nc2cc(NC(=O)C3CCC3)ncc2C(C)=O)cccc1-c1ncn(C)n1] |
| 1K | 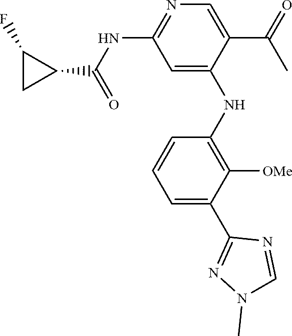 [SMILES: COc1c(Nc2cc(NC(=O)[C@@H]3C[C@@H]3F)ncc2C(C)=O)cccc1-c1ncn(C)n1] |
| 1L | 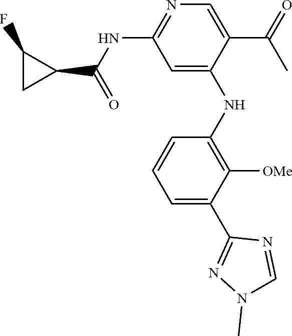 [SMILES: COc1c(Nc2cc(NC(=O)[C@H]3C[C@H]3F)ncc2C(C)=O)cccc1-c1ncn(C)n1] |
| 1M | 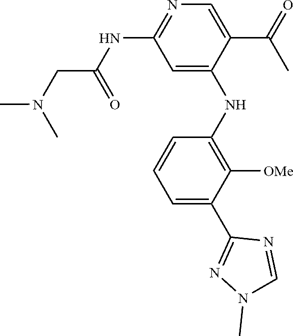 [SMILES: COc1c(Nc2cc(NC(=O)CN(C)C)ncc2C(C)=O)cccc1-c1ncn(C)n1] |
| 1N | 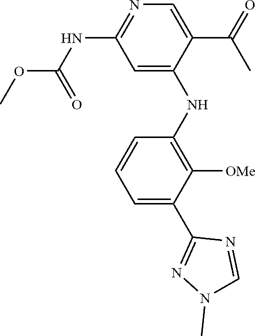 [SMILES: COC(=O)Nc1cc(Nc2cccc(-c3ncn(C)n3)c2OC)c(C(C)=O)cn1] |
| 1O | 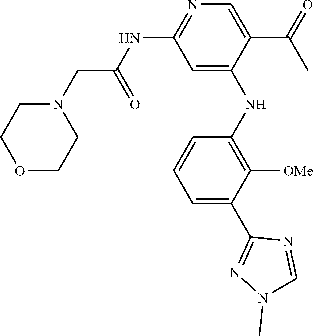 [SMILES: COc1c(Nc2cc(NC(=O)CN3CCOCC3)ncc2C(C)=O)cccc1-c1ncn(C)n1] |
| 1P | 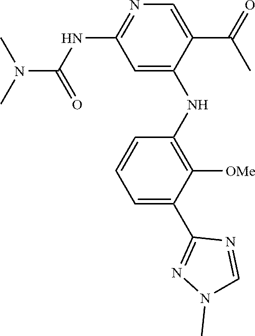 [SMILES: COc1c(Nc2cc(NC(=O)N(C)C)ncc2C(C)=O)cccc1-c1ncn(C)n1] |
| 2 | 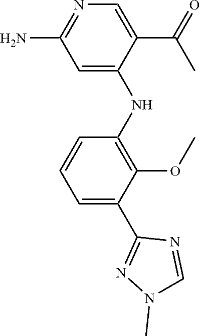 [SMILES: COc1c(Nc2cc(N)ncc2C(C)=O)cccc1-c1ncn(C)n1] |
| 3 | 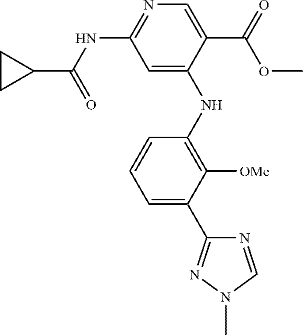 [SMILES: COC(=O)c1cnc(NC(=O)C2CC2)cc1Nc1cccc(-c2ncn(C)n2)c1OC] |
| 4A | 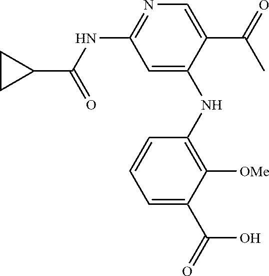 [SMILES: COc1c(Nc2cc(NC(=O)C3CC3)ncc2C(C)=O)cccc1C(=O)O] |
| 4B | 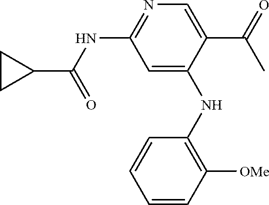 [SMILES: COc1ccccc1Nc1cc(NC(=O)C2CC2)ncc1C(C)=O] |
| 4C | 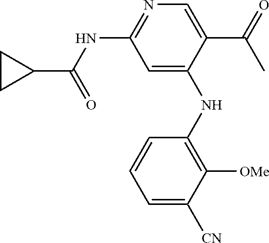 [SMILES: COc1c(C#N)cccc1Nc1cc(NC(=O)C2CC2)ncc1C(C)=O] |
| 4D | 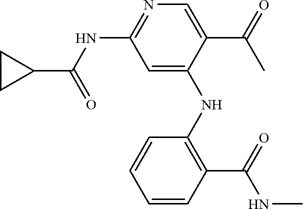 [SMILES: CNC(=O)c1ccccc1Nc1cc(NC(=O)C2CC2)ncc1C(C)=O] |
| 4E | 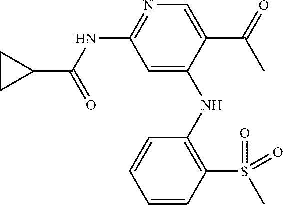 [SMILES: CC(=O)c1cnc(NC(=O)C2CC2)cc1Nc1ccccc1S(C)(=O)=O] |
| 5A | 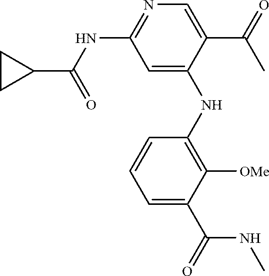 [SMILES: CNC(=O)c1cccc(Nc2cc(NC(=O)C3CC3)ncc2C(C)=O)c1OC] |
| 5B | 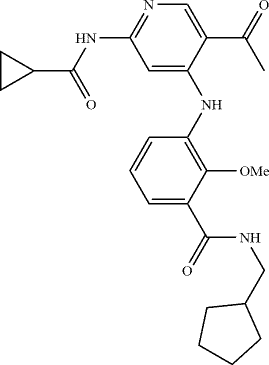 [SMILES: COc1c(Nc2cc(NC(=O)C3CC3)ncc2C(C)=O)cccc1C(=O)NCC1CCCC1] |
| 5C | 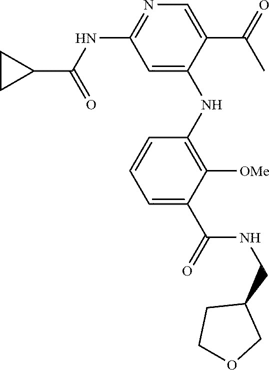 [SMILES: COc1c(Nc2cc(NC(=O)C3CC3)ncc2C(C)=O)cccc1C(=O)NC[C@@H]1CCOC1] |
| 5D | 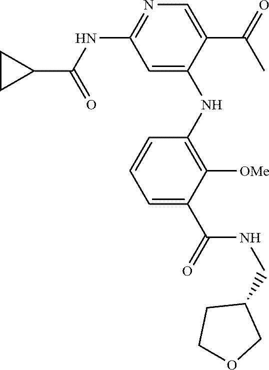 [SMILES: COc1c(Nc2cc(NC(=O)C3CC3)ncc2C(C)=O)cccc1C(=O)NC[C@H]1CCOC1] |
| 5E | 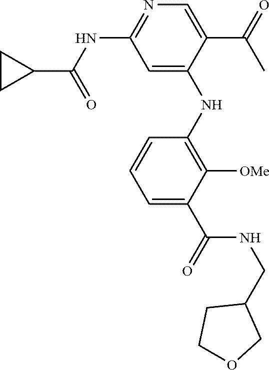 [SMILES: COc1c(Nc2cc(NC(=O)C3CC3)ncc2C(C)=O)cccc1C(=O)NCC1CCOC1] |
| 5F | 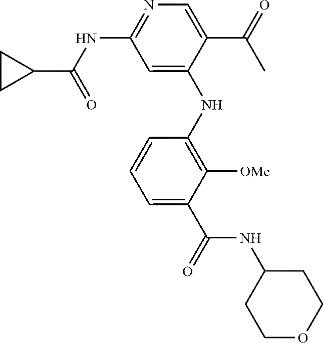 [SMILES: COc1c(Nc2cc(NC(=O)C3CC3)ncc2C(C)=O)cccc1C(=O)NC1CCOCC1] |
| 6A | 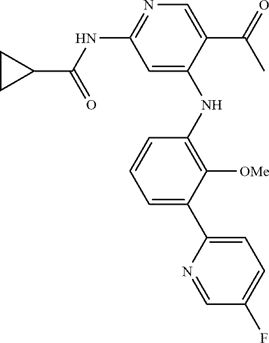 [SMILES: COc1c(Nc2cc(NC(=O)C3CC3)ncc2C(C)=O)cccc1-c1ccc(F)cn1] |
| 6B | 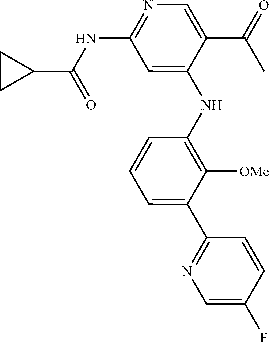 [SMILES: COc1c(Nc2cc(NC(=O)C3CC3)ncc2C(C)=O)cccc1-c1ccc(F)cn1] |
| 6C | 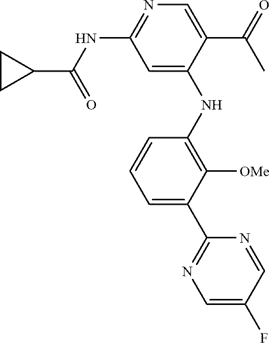 [SMILES: COc1c(Nc2cc(NC(=O)C3CC3)ncc2C(C)=O)cccc1-c1ncc(F)cn1] |
| 6D | 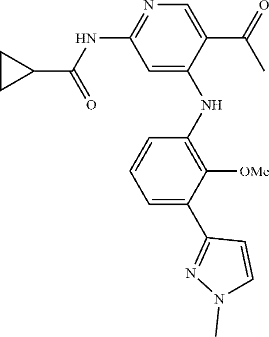 [SMILES: COc1c(Nc2cc(NC(=O)C3CC3)ncc2C(C)=O)cccc1-c1ccn(C)n1] |
| 7A | 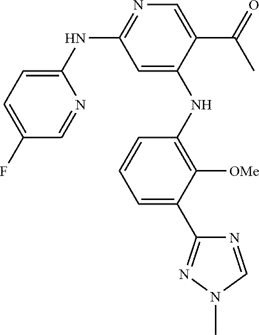 [SMILES: COc1c(Nc2cc(Nc3ccc(F)cn3)ncc2C(C)=O)cccc1-c1ncn(C)n1] |
| 7B | 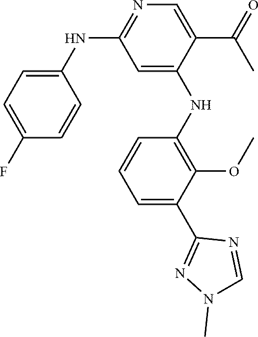 [SMILES: COc1c(Nc2cc(Nc3ccc(F)cc3)ncc2C(C)=O)cccc1-c1ncn(C)n1] |
| 7C | 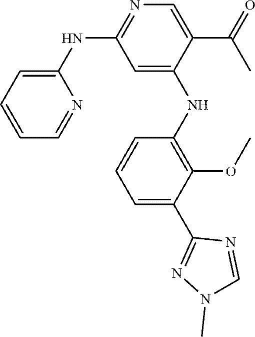 [SMILES: COc1c(Nc2cc(Nc3ccccn3)ncc2C(C)=O)cccc1-c1ncn(C)n1] |
| 7D | 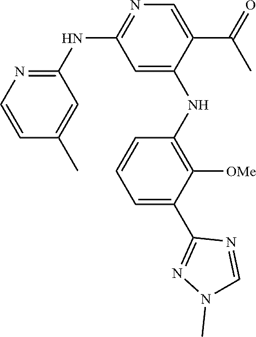 [SMILES: COc1c(Nc2cc(Nc3cc(C)ccn3)ncc2C(C)=O)cccc1-c1ncn(C)n1] |
| 7E | 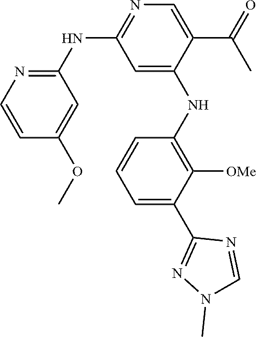 [SMILES: COc1ccnc(Nc2cc(Nc3cccc(-c4ncn(C)n4)c3OC)c(C(C)=O)cn2)c1] |
| 7F | 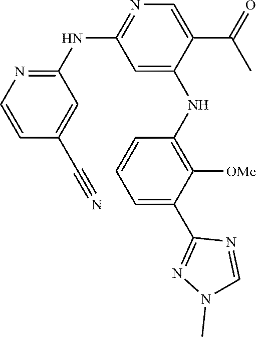 [SMILES: COc1c(Nc2cc(Nc3cc(C#N)ccn3)ncc2C(C)=O)cccc1-c1ncn(C)n1] |
| 7G | 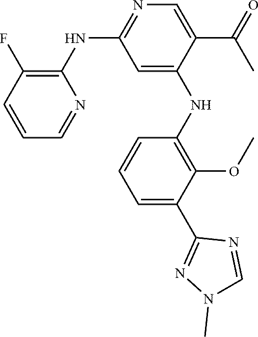 [SMILES: COc1c(Nc2cc(Nc3ncccc3F)ncc2C(C)=O)cccc1-c1ncn(C)n1] |
| 7H | 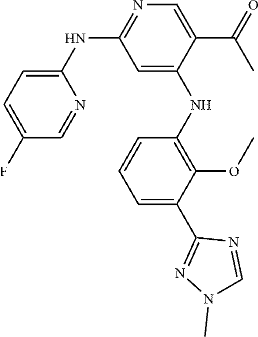 [SMILES: COc1c(Nc2cc(Nc3ccc(F)cn3)ncc2C(C)=O)cccc1-c1ncn(C)n1] |
| 7I | 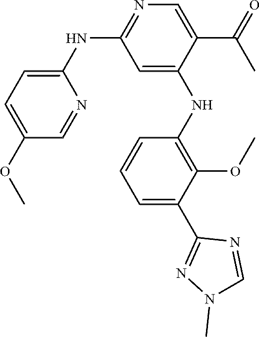 [SMILES: COc1ccc(Nc2cc(Nc3cccc(-c4ncn(C)n4)c3OC)c(C(C)=O)cn2)nc1] |
| 7J | 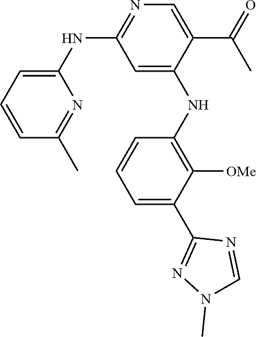 [SMILES: COc1c(Nc2cc(Nc3cccc(C)n3)ncc2C(C)=O)cccc1-c1ncn(C)n1] |
| 7K | 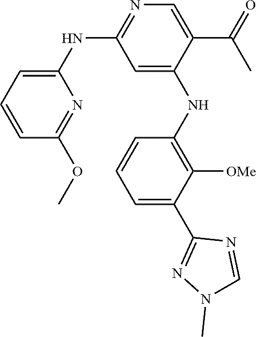 [SMILES: COc1cccc(Nc2cc(Nc3cccc(-c4ncn(C)n4)c3OC)c(C(C)=O)cn2)n1] |
| 7L | 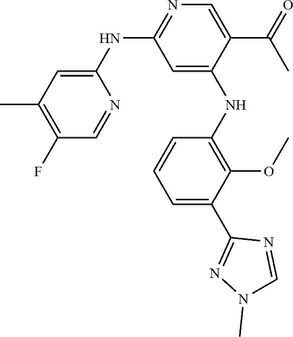 |
| 7M | 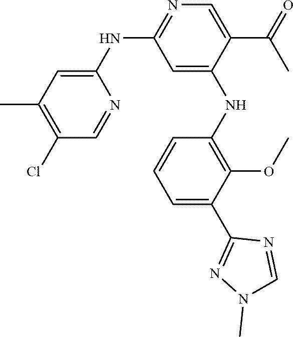 |
| 7N | 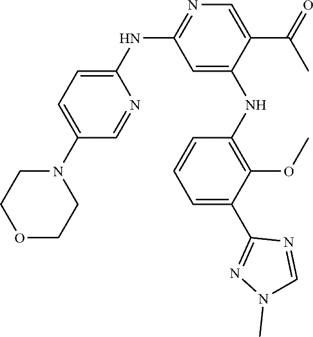 [SMILES: COc1c(Nc2cc(Nc3ccc(N4CCOCC4)cn3)ncc2C(C)=O)cccc1-c1ncn(C)n1] |
| 7O | 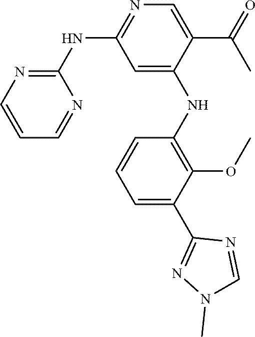 [SMILES: COc1c(Nc2cc(Nc3ncccn3)ncc2C(C)=O)cccc1-c1ncn(C)n1] |
| 7P | 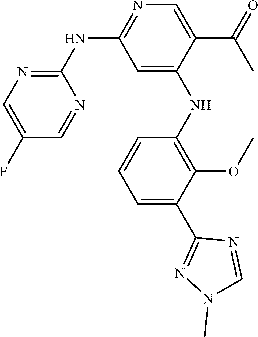 [SMILES: COc1c(Nc2cc(Nc3ncc(F)cn3)ncc2C(C)=O)cccc1-c1ncn(C)n1] |
| 7Q | 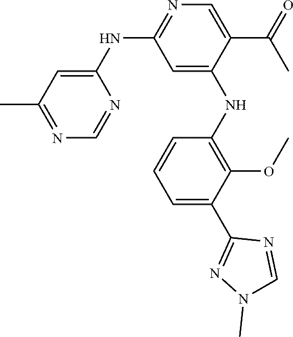 |
| 7R | 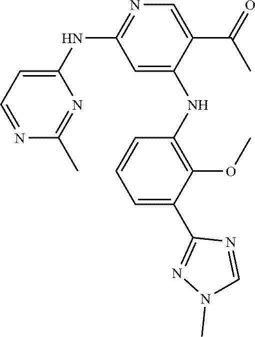 [SMILES: COc1c(Nc2cc(Nc3ccnc(C)n3)ncc2C(C)=O)cccc1-c1ncn(C)n1] |
| 7S | 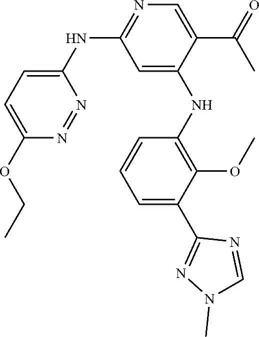 [SMILES: CCOc1ccc(Nc2cc(Nc3cccc(-c4ncn(C)n4)c3OC)c(C(C)=O)cn2)nn1] |
| 7T | 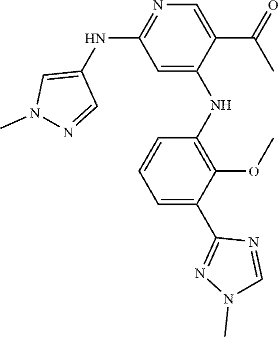 |
| 7U | 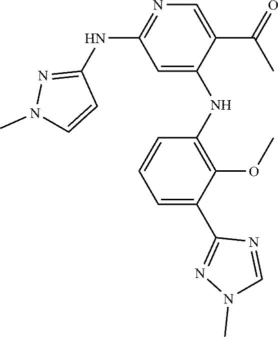 |
| 8A | 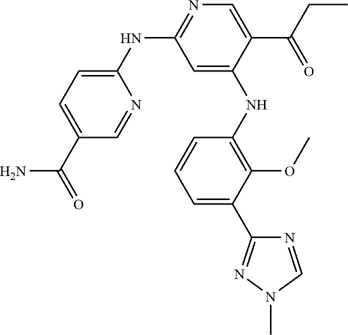 [SMILES: CCC(=O)c1cnc(Nc2ccc(C(N)=O)cn2)cc1Nc1cccc(-c2ncn(C)n2)c1OC] |
| 8B | 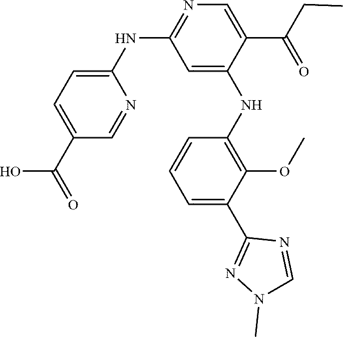 [SMILES: CCC(=O)c1cnc(Nc2ccc(C(=O)O)cn2)cc1Nc1cccc(-c2ncn(C)n2)c1OC] |
| 8C | 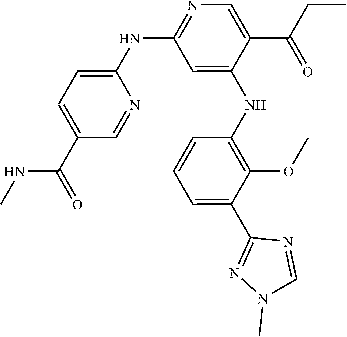 [SMILES: CCC(=O)c1cnc(Nc2ccc(C(=O)NC)cn2)cc1Nc1cccc(-c2ncn(C)n2)c1OC] |
| 8D | 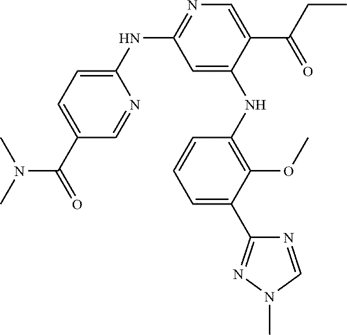 [SMILES: CCC(=O)c1cnc(Nc2ccc(C(=O)N(C)C)cn2)cc1Nc1cccc(-c2ncn(C)n2)c1OC] |
| 8E | 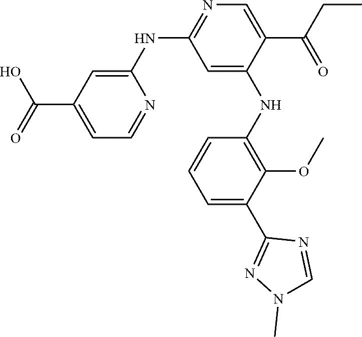 [SMILES: CCC(=O)c1cnc(Nc2cc(C(=O)O)ccn2)cc1Nc1cccc(-c2ncn(C)n2)c1OC] |
| 8F | 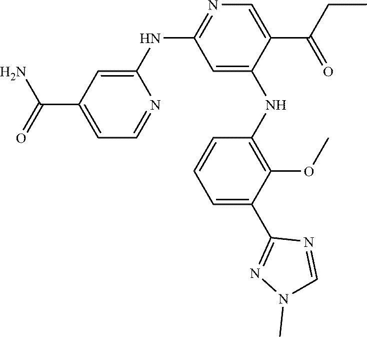 [SMILES: CCC(=O)c1cnc(Nc2cc(C(N)=O)ccn2)cc1Nc1cccc(-c2ncn(C)n2)c1OC] |
| 8G | 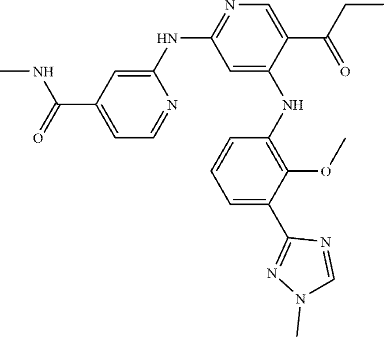 [SMILES: CCC(=O)c1cnc(Nc2cc(C(=O)NC)ccn2)cc1Nc1cccc(-c2ncn(C)n2)c1OC] |
| 8H | 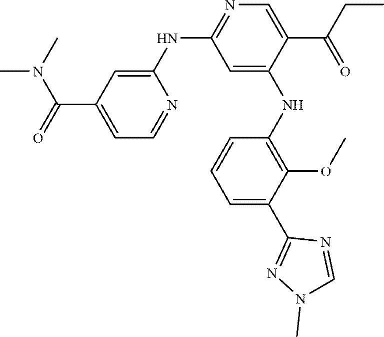 [SMILES: CCC(=O)c1cnc(Nc2cc(C(=O)N(C)C)ccn2)cc1Nc1cccc(-c2ncn(C)n2)c1OC] |
| 8I | 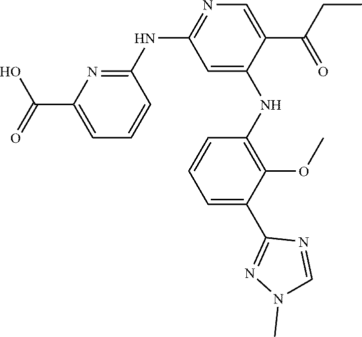 [SMILES: CCC(=O)c1cnc(Nc2cccc(C(=O)O)n2)cc1Nc1cccc(-c2ncn(C)n2)c1OC] |
| 8J | 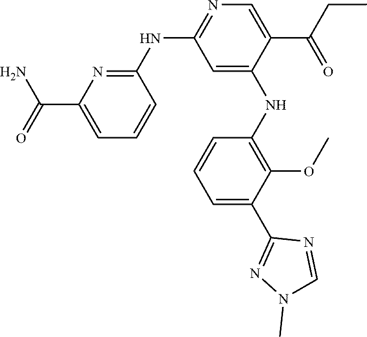 [SMILES: CCC(=O)c1cnc(Nc2cccc(C(N)=O)n2)cc1Nc1cccc(-c2ncn(C)n2)c1OC] |
| 8K | 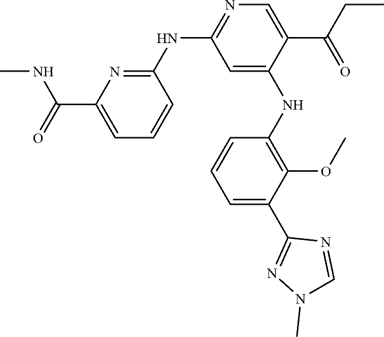 [SMILES: CCC(=O)c1cnc(Nc2cccc(C(=O)NC)n2)cc1Nc1cccc(-c2ncn(C)n2)c1OC] |
| 8L | 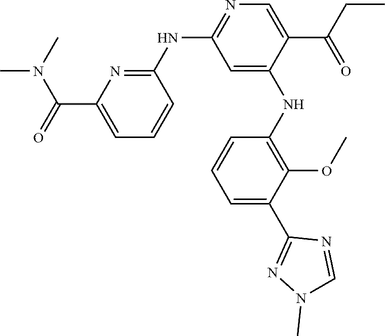 [SMILES: CCC(=O)c1cnc(Nc2cccc(C(=O)N(C)C)n2)cc1Nc1cccc(-c2ncn(C)n2)c1OC] |
| 8M | 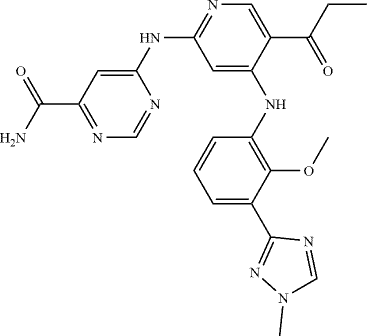 [SMILES: CCC(=O)c1cnc(Nc2cc(C(N)=O)ncn2)cc1Nc1cccc(-c2ncn(C)n2)c1OC] |
| 8N | 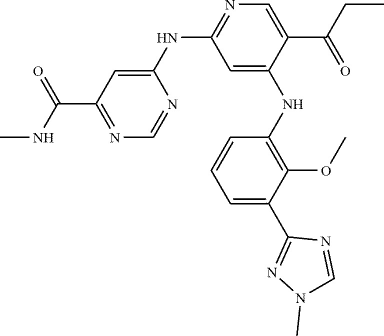 [SMILES: CCC(=O)c1cnc(Nc2cc(C(=O)NC)ncn2)cc1Nc1cccc(-c2ncn(C)n2)c1OC] |
| 8O | 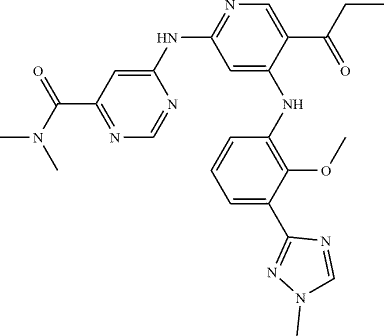 [SMILES: CCC(=O)c1cnc(Nc2cc(C(=O)N(C)C)ncn2)cc1Nc1cccc(-c2ncn(C)n2)c1OC] |
| 9A | 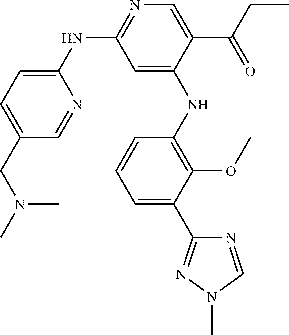 [SMILES: CCC(=O)c1cnc(Nc2ccc(CN(C)C)cn2)cc1Nc1cccc(-c2ncn(C)n2)c1OC] |
| 9B | 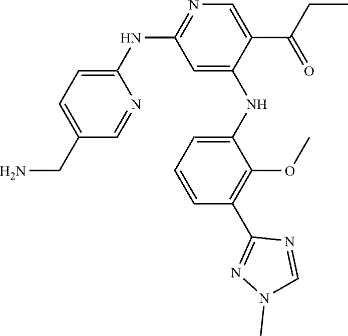 [SMILES: CCC(=O)c1cnc(Nc2ccc(CN)cn2)cc1Nc1cccc(-c2ncn(C)n2)c1OC] |
| 9C | 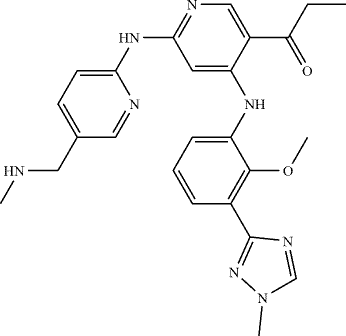 [SMILES: CCC(=O)c1cnc(Nc2ccc(CNC)cn2)cc1Nc1cccc(-c2ncn(C)n2)c1OC] |
| 9D | 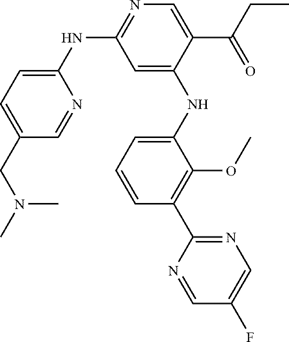 [SMILES: CCC(=O)c1cnc(Nc2ccc(CN(C)C)cn2)cc1Nc1cccc(-c2ncc(F)cn2)c1OC] |
| 9E | 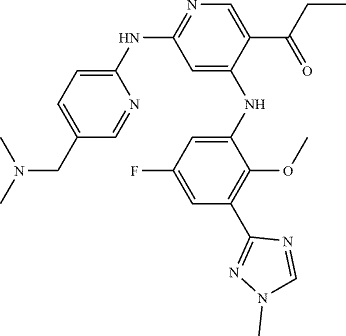 [SMILES: CCC(=O)c1cnc(Nc2ccc(CN(C)C)cn2)cc1Nc1cc(F)cc(-c2ncn(C)n2)c1OC] |
| 9F | 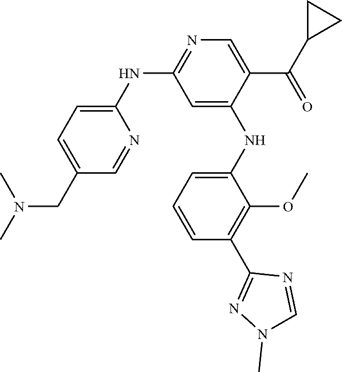 [SMILES: COc1c(Nc2cc(Nc3ccc(CN(C)C)cn3)ncc2C(=O)C2CC2)cccc1-c1ncn(C)n1] |
| 9G | 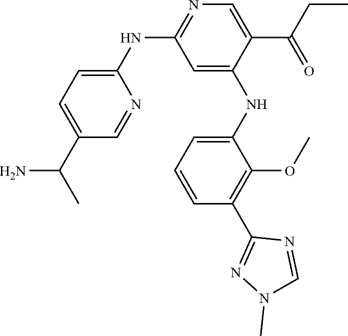 [SMILES: CCC(=O)c1cnc(Nc2ccc(C(C)N)cn2)cc1Nc1cccc(-c2ncn(C)n2)c1OC] |
| 9H | 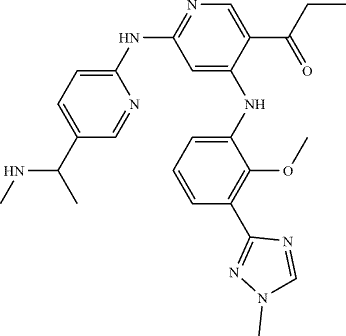 [SMILES: CCC(=O)c1cnc(Nc2ccc(C(C)NC)cn2)cc1Nc1cccc(-c2ncn(C)n2)c1OC] |
| 9I | 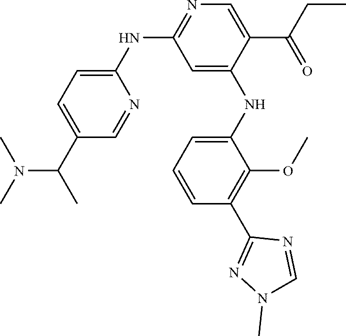 [SMILES: CCC(=O)c1cnc(Nc2ccc(C(C)N(C)C)cn2)cc1Nc1cccc(-c2ncn(C)n2)c1OC] |
| 9J | 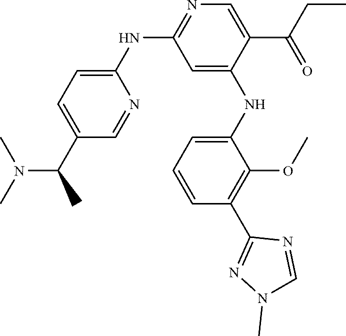 [SMILES: CCC(=O)c1cnc(Nc2ccc([C@@H](C)N(C)C)cn2)cc1Nc1cccc(-c2ncn(C)n2)c1OC] |
| 9K | 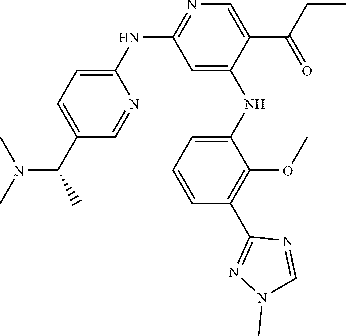 [SMILES: CCC(=O)c1cnc(Nc2ccc([C@H](C)N(C)C)cn2)cc1Nc1cccc(-c2ncn(C)n2)c1OC] |
| 9L | 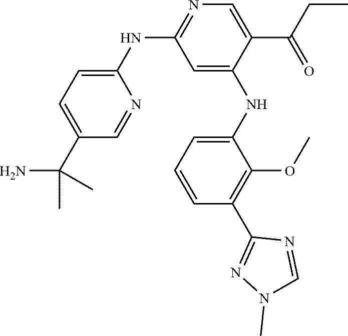 [SMILES: CCC(=O)c1cnc(Nc2ccc(C(C)(C)N)cn2)cc1Nc1cccc(-c2ncn(C)n2)c1OC] |
| 9M | 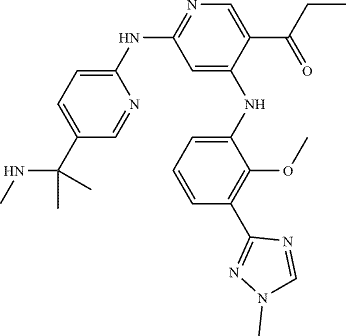 [SMILES: CCC(=O)c1cnc(Nc2ccc(C(C)(C)NC)cn2)cc1Nc1cccc(-c2ncn(C)n2)c1OC] |
| 9N | 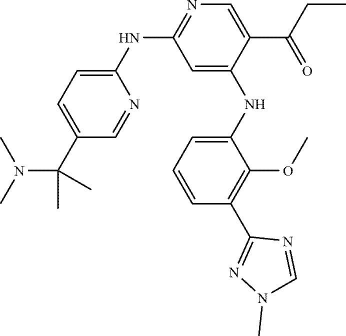 [SMILES: CCC(=O)c1cnc(Nc2ccc(C(C)(C)N(C)C)cn2)cc1Nc1cccc(-c2ncn(C)n2)c1OC] |
| 9O | 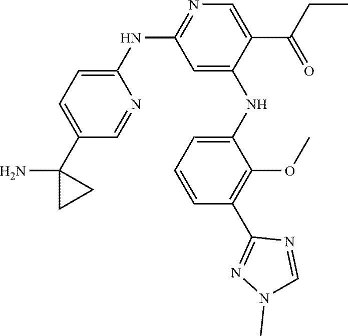 [SMILES: CCC(=O)c1cnc(Nc2ccc(C3(N)CC3)cn2)cc1Nc1cccc(-c2ncn(C)n2)c1OC] |
| 9P | 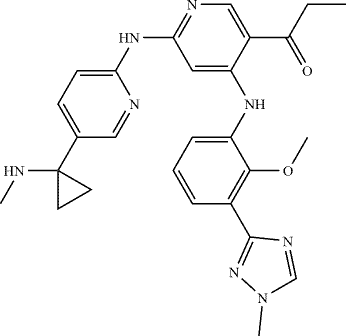 [SMILES: CCC(=O)c1cnc(Nc2ccc(C3(NC)CC3)cn2)cc1Nc1cccc(-c2ncn(C)n2)c1OC] |
| 9Q | 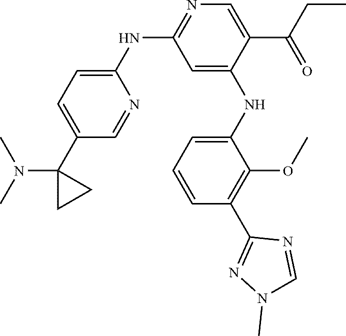 [SMILES: CCC(=O)c1cnc(Nc2ccc(C3(N(C)C)CC3)cn2)cc1Nc1cccc(-c2ncn(C)n2)c1OC] |
| 9R | 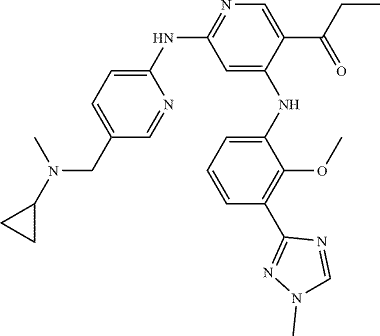 [SMILES: CCC(=O)c1cnc(Nc2ccc(CN(C)C3CC3)cn2)cc1Nc1cccc(-c2ncn(C)n2)c1OC] |
| 9S | 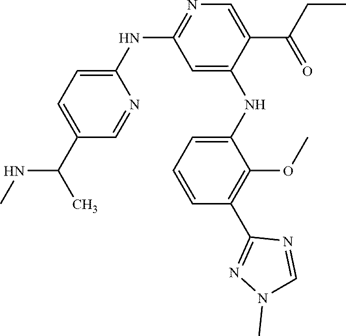 [SMILES: CCC(=O)c1cnc(Nc2ccc(C(C)NC)cn2)cc1Nc1cccc(-c2ncn(C)n2)c1OC] |
| 9T | 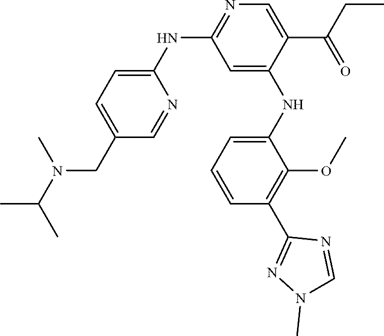 [SMILES: CCC(=O)c1cnc(Nc2ccc(CN(C)C(C)C)cn2)cc1Nc1cccc(-c2ncn(C)n2)c1OC] |
| 9U | 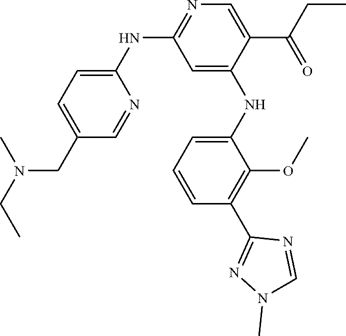 [SMILES: CCC(=O)c1cnc(Nc2ccc(CN(C)CC)cn2)cc1Nc1cccc(-c2ncn(C)n2)c1OC] |
| 9V | 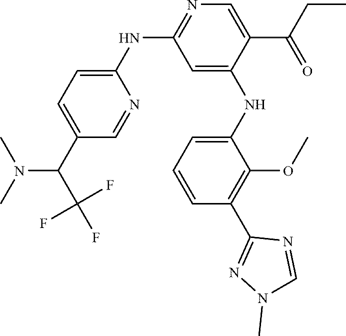 [SMILES: CCC(=O)c1cnc(Nc2ccc(C(N(C)C)C(F)(F)F)cn2)cc1Nc1cccc(-c2ncn(C)n2)c1OC] |
| 9W | 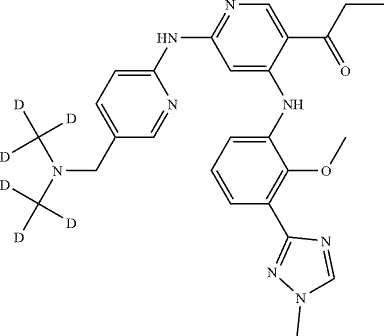 [SMILES: [2H]C([2H])([2H])N(Cc1ccc(Nc2cc(Nc3cccc(-c4ncn(C)n4)c3OC)c(C(=O)CC)cn2)nc1)C([2H])([2H])[2H]] |
| 9X | 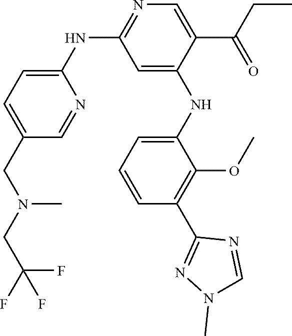 [SMILES: CCC(=O)c1cnc(Nc2ccc(CN(C)CC(F)(F)F)cn2)cc1Nc1cccc(-c2ncn(C)n2)c1OC] |
| 9Y | 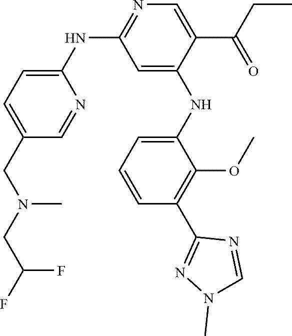 [SMILES: CCC(=O)c1cnc(Nc2ccc(CN(C)CC(F)F)cn2)cc1Nc1cccc(-c2ncn(C)n2)c1OC] |
| 9Z | 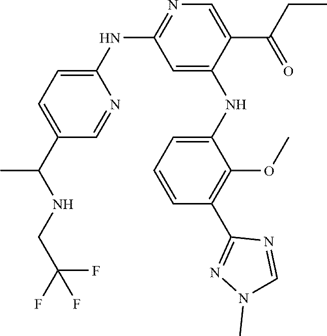 [SMILES: CCC(=O)c1cnc(Nc2ccc(C(C)NCC(F)(F)F)cn2)cc1Nc1cccc(-c2ncn(C)n2)c1OC] |
| 9 AA | 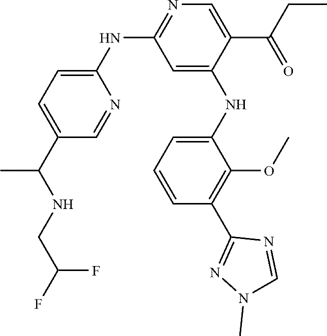 [SMILES: CCC(=O)c1cnc(Nc2ccc(C(C)NCC(F)F)cn2)cc1Nc1cccc(-c2ncn(C)n2)c1OC] |
| 9BB | 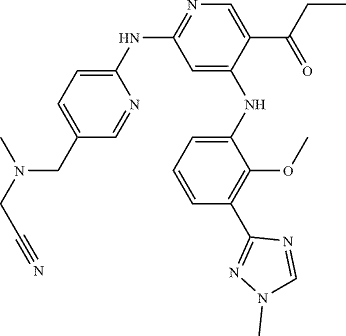 [SMILES: CCC(=O)c1cnc(Nc2ccc(CN(C)CC#N)cn2)cc1Nc1cccc(-c2ncn(C)n2)c1OC] |
| 9CC | 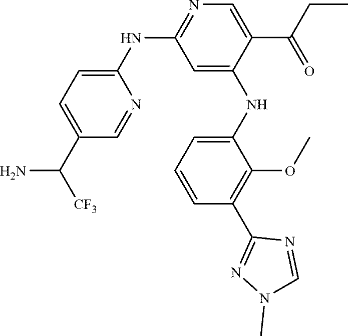 [SMILES: CCC(=O)c1cnc(Nc2ccc(C(N)C(F)(F)F)cn2)cc1Nc1cccc(-c2ncn(C)n2)c1OC] |
| 9 DD | 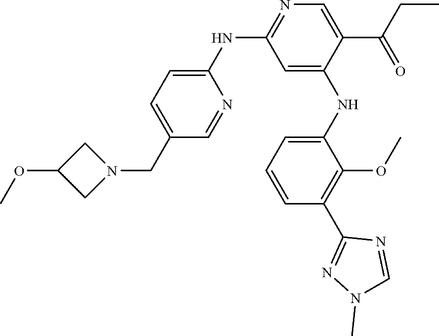 [SMILES: CCC(=O)c1cnc(Nc2ccc(CN3CC(OC)C3)cn2)cc1Nc1cccc(-c2ncn(C)n2)c1OC] |
| 9 EE | 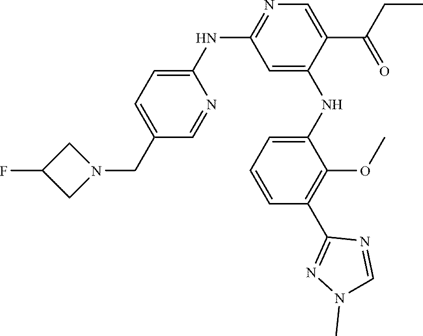 [SMILES: CCC(=O)c1cnc(Nc2ccc(CN3CC(F)C3)cn2)cc1Nc1cccc(-c2ncn(C)n2)c1OC] |
| 9 FF | 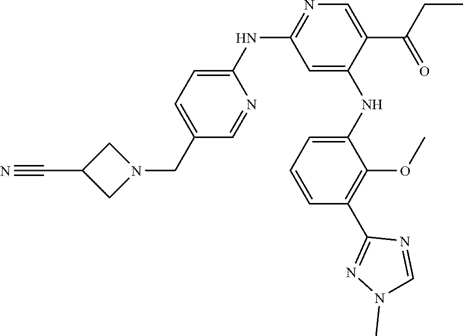 [SMILES: CCC(=O)c1cnc(Nc2ccc(CN3CC(C#N)C3)cn2)cc1Nc1cccc(-c2ncn(C)n2)c1OC] |
| 9GG | 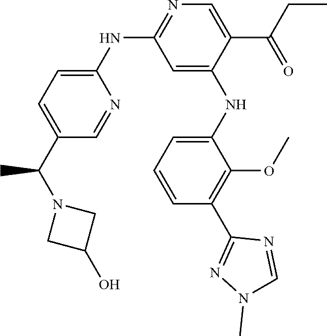 [SMILES: CCC(=O)c1cnc(Nc2ccc([C@H](C)N3CC(O)C3)cn2)cc1Nc1cccc(-c2ncn(C)n2)c1OC] |
| 9 HH | 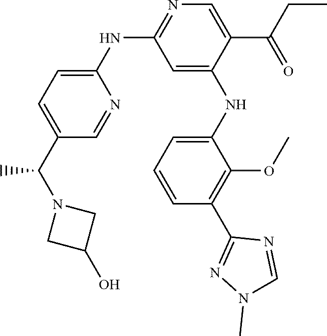 [SMILES: CCC(=O)c1cnc(Nc2ccc([C@@H](C)N3CC(O)C3)cn2)cc1Nc1cccc(-c2ncn(C)n2)c1OC] |
| 9 II | 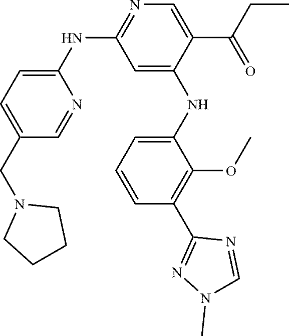 [SMILES: CCC(=O)c1cnc(Nc2ccc(CN3CCCC3)cn2)cc1Nc1cccc(-c2ncn(C)n2)c1OC] |
| 9 JJ | 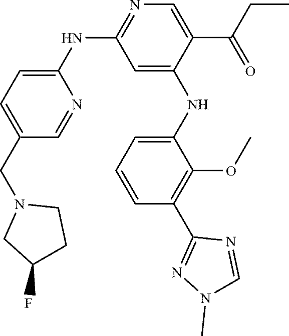 [SMILES: CCC(=O)c1cnc(Nc2ccc(CN3CC[C@@H](F)C3)cn2)cc1Nc1cccc(-c2ncn(C)n2)c1OC] |
| 9 KK | 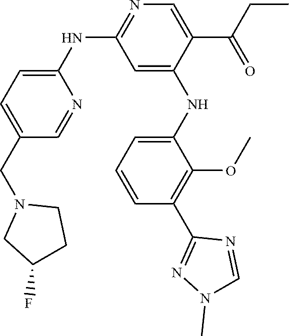 [SMILES: CCC(=O)c1cnc(Nc2ccc(CN3CC[C@H](F)C3)cn2)cc1Nc1cccc(-c2ncn(C)n2)c1OC] |
| 9 LL | |
| 9 MM | 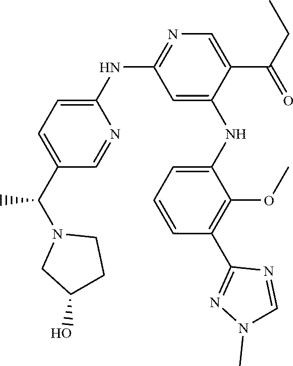 [SMILES: CCC(=O)c1cnc(Nc2ccc([C@@H](C)N3CC[C@H](O)C3)cn2)cc1Nc1cccc(-c2ncn(C)n2)c1OC] |
| 9 NN | 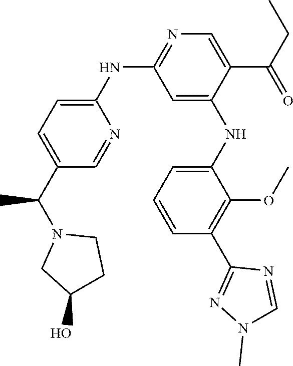 [SMILES: CCC(=O)c1cnc(Nc2ccc([C@H](C)N3CC[C@@H](O)C3)cn2)cc1Nc1cccc(-c2ncn(C)n2)c1OC] |
| 9OO | 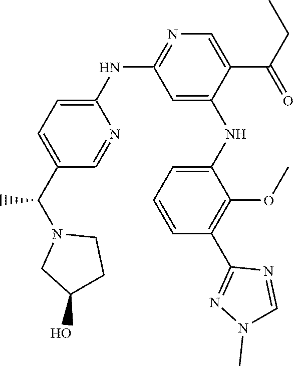 [SMILES: CCC(=O)c1cnc(Nc2ccc([C@@H](C)N3CC[C@@H](O)C3)cn2)cc1Nc1cccc(-c2ncn(C)n2)c1OC] |
| 9PP | 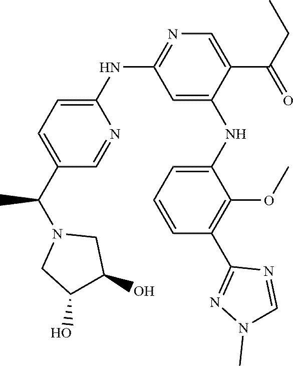 [SMILES: CCC(=O)c1cnc(Nc2ccc([C@H](C)N3C[C@@H](O)[C@H](O)C3)cn2)cc1Nc1cccc(-c2ncn(C)n2)c1OC] |
| 9 QQ | 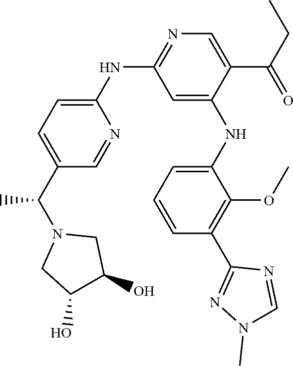 [SMILES: CCC(=O)c1cnc(Nc2ccc([C@@H](C)N3C[C@@H](O)[C@H](O)C3)cn2)cc1Nc1cccc(-c2ncn(C)n2)c1OC] |
| 9 RR | 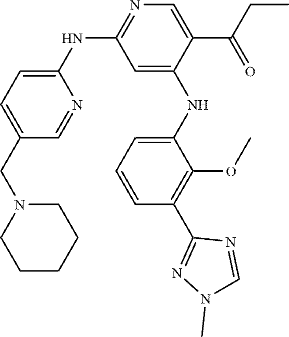 [SMILES: CCC(=O)c1cnc(Nc2ccc(CN3CCCCC3)cn2)cc1Nc1cccc(-c2ncn(C)n2)c1OC] |
| 9 SS | 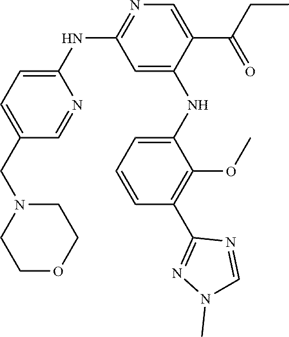 [SMILES: CCC(=O)c1cnc(Nc2ccc(CN3CCOCC3)cn2)cc1Nc1cccc(-c2ncn(C)n2)c1OC] |
| 9 TT | 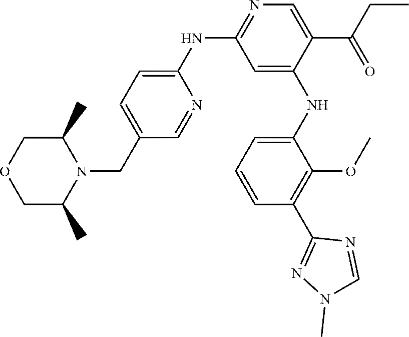 [SMILES: CCC(=O)c1cnc(Nc2ccc(CN3[C@H](C)COC[C@@H]3C)cn2)cc1Nc1cccc(-c2ncn(C)n2)c1OC] |
| 9UU | 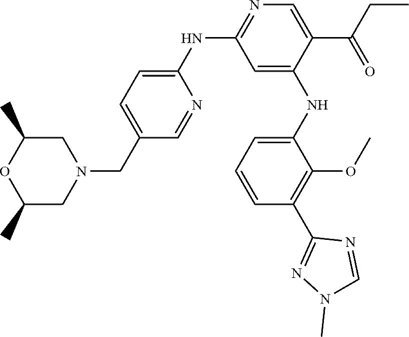 [SMILES: CCC(=O)c1cnc(Nc2ccc(CN3C[C@@H](C)O[C@@H](C)C3)cn2)cc1Nc1cccc(-c2ncn(C)n2)c1OC] |
| 9 VV | 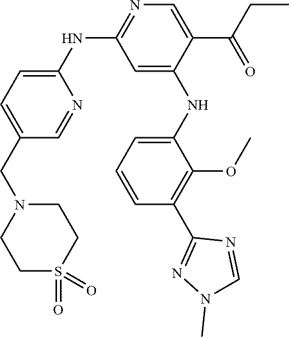 [SMILES: CCC(=O)c1cnc(Nc2ccc(CN3CCS(=O)(=O)CC3)cn2)cc1Nc1cccc(-c2ncn(C)n2)c1OC] |
| 9WW | 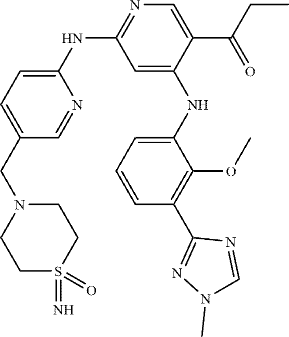 [SMILES: CCC(=O)c1cnc(Nc2ccc(CN3CCS(=N)(=O)CC3)cn2)cc1Nc1cccc(-c2ncn(C)n2)c1OC] |
| 9 XX | 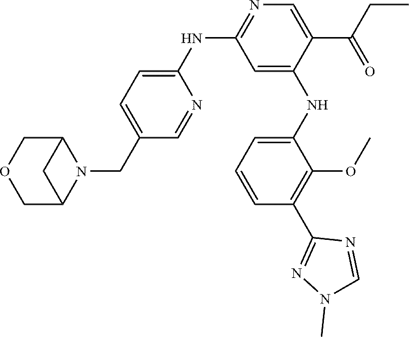 [SMILES: CCC(=O)c1cnc(Nc2ccc(CN3C4COCC3C4)cn2)cc1Nc1cccc(-c2ncn(C)n2)c1OC] |
| 9 YY | 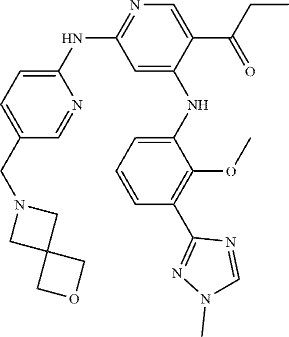 [SMILES: CCC(=O)c1cnc(Nc2ccc(CN3CC4(COC4)C3)cn2)cc1Nc1cccc(-c2ncn(C)n2)c1OC] |
| 9 ZZ | 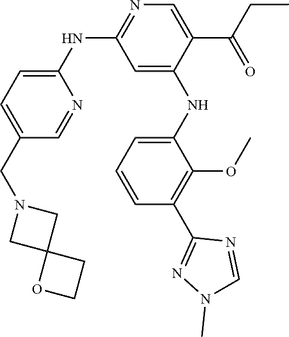 [SMILES: CCC(=O)c1cnc(Nc2ccc(CN3CC4(CCO4)C3)cn2)cc1Nc1cccc(-c2ncn(C)n2)c1OC] |
| 9 AAA | 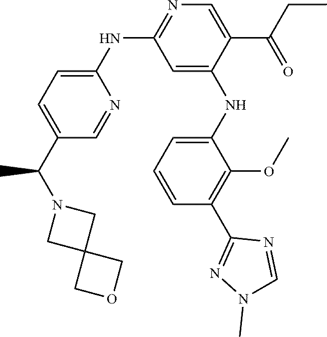 [SMILES: CCC(=O)c1cnc(Nc2ccc([C@H](C)N3CC4(COC4)C3)cn2)cc1Nc1cccc(-c2ncn(C)n2)c1OC] |
| 9 BBB | 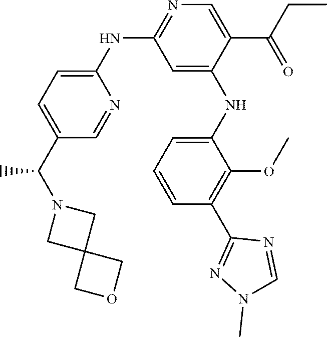 [SMILES: CCC(=O)c1cnc(Nc2ccc([C@@H](C)N3CC4(COC4)C3)cn2)cc1Nc1cccc(-c2ncn(C)n2)c1OC] |
| 9CCC | 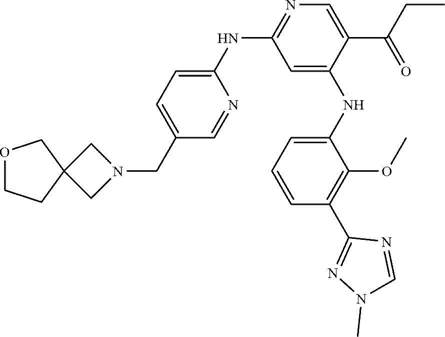 [SMILES: CCC(=O)c1cnc(Nc2ccc(CN3CC4(CCOC4)C3)cn2)cc1Nc1cccc(-c2ncn(C)n2)c1OC] |
| 9 DDD | 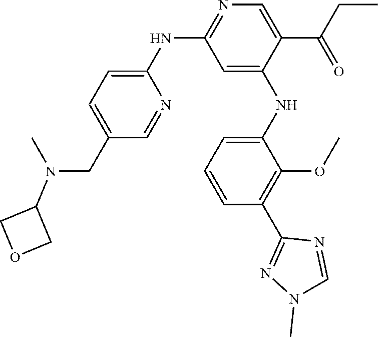 [SMILES: CCC(=O)c1cnc(Nc2ccc(CN(C)C3COC3)cn2)cc1Nc1cccc(-c2ncn(C)n2)c1OC] |
| 9 EEE | 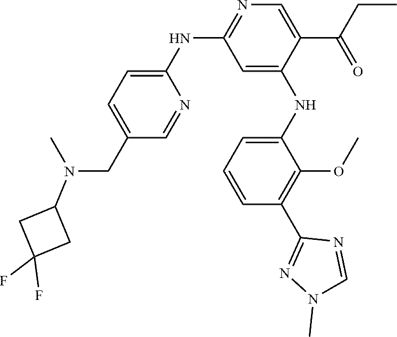 [SMILES: CCC(=O)c1cnc(Nc2ccc(CN(C)C3CC(F)(F)C3)cn2)cc1Nc1cccc(-c2ncn(C)n2)c1OC] |
| 9FFF | 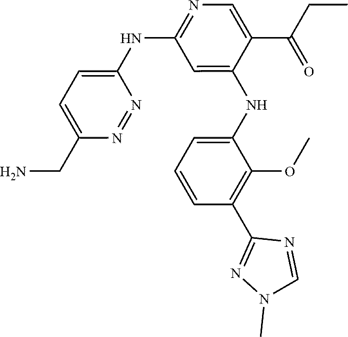 [SMILES: CCC(=O)c1cnc(Nc2ccc(CN)nn2)cc1Nc1cccc(-c2ncn(C)n2)c1OC] |
| 9 GGG | 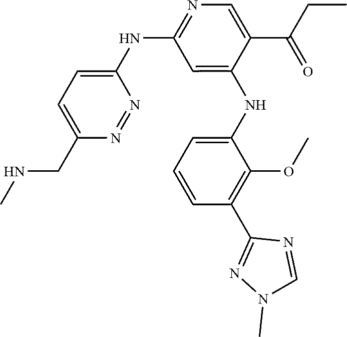 [SMILES: CCC(=O)c1cnc(Nc2ccc(CNC)nn2)cc1Nc1cccc(-c2ncn(C)n2)c1OC] |
| 9 HHH | 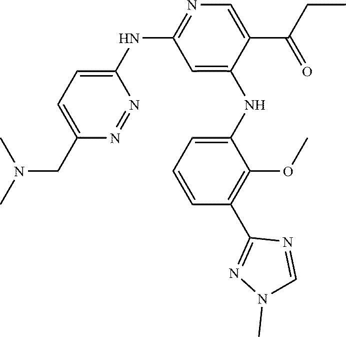 [SMILES: CCC(=O)c1cnc(Nc2ccc(CN(C)C)nn2)cc1Nc1cccc(-c2ncn(C)n2)c1OC] |
| 9 III | 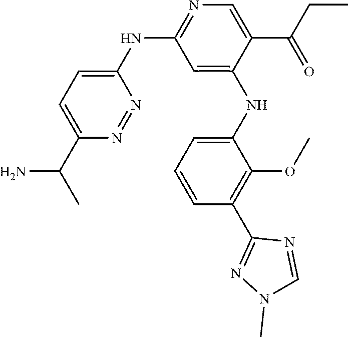 [SMILES: CCC(=O)c1cnc(Nc2ccc(C(C)N)nn2)cc1Nc1cccc(-c2ncn(C)n2)c1OC] |
| 9 JJJ | 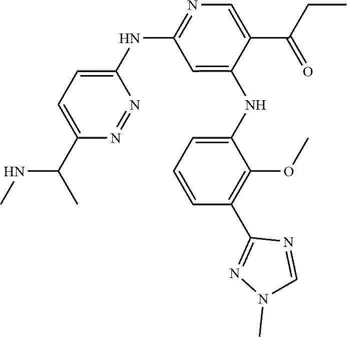 [SMILES: CCC(=O)c1cnc(Nc2ccc(C(C)NC)nn2)cc1Nc1cccc(-c2ncn(C)n2)c1OC] |
| 9 KKK | 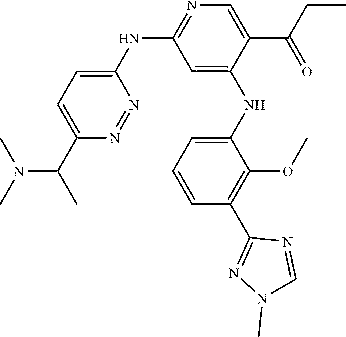 [SMILES: CCC(=O)c1cnc(Nc2ccc(C(C)N(C)C)nn2)cc1Nc1cccc(-c2ncn(C)n2)c1OC] |
| 9 LLL | 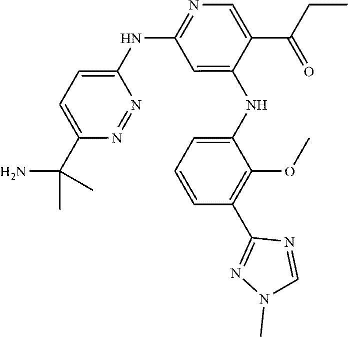 [SMILES: CCC(=O)c1cnc(Nc2ccc(C(C)(C)N)nn2)cc1Nc1cccc(-c2ncn(C)n2)c1OC] |
| 9 MMM | 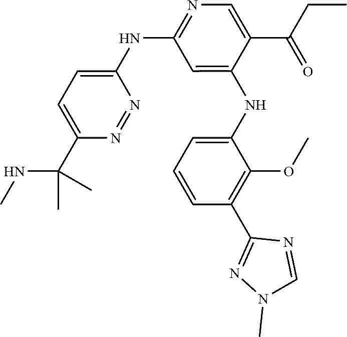 [SMILES: CCC(=O)c1cnc(Nc2ccc(C(C)(C)NC)nn2)cc1Nc1cccc(-c2ncn(C)n2)c1OC] |
| 9 NNN | 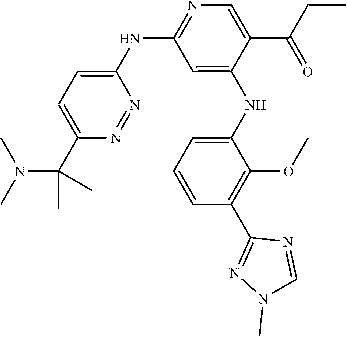 [SMILES: CCC(=O)c1cnc(Nc2ccc(C(C)(C)N(C)C)nn2)cc1Nc1cccc(-c2ncn(C)n2)c1OC] |
| 9OOO | 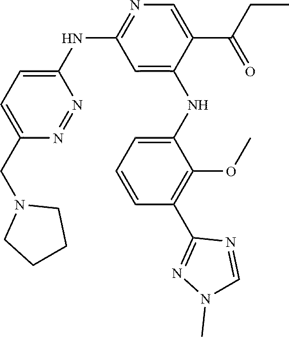 [SMILES: CCC(=O)c1cnc(Nc2ccc(CN3CCCC3)nn2)cc1Nc1cccc(-c2ncn(C)n2)c1OC] |
| 9 PPP | 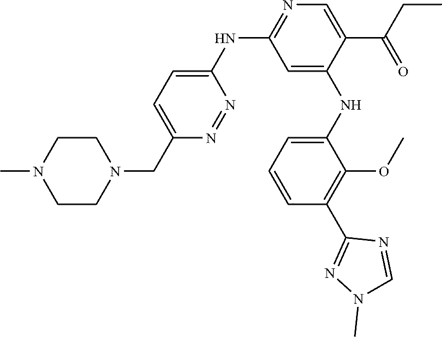 [SMILES: CCC(=O)c1cnc(Nc2ccc(CN3CCN(C)CC3)nn2)cc1Nc1cccc(-c2ncn(C)n2)c1OC] |
| 9 QQQ | 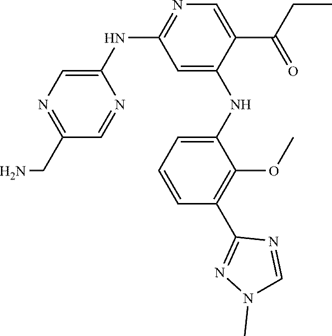 [SMILES: CCC(=O)c1cnc(Nc2cnc(CN)cn2)cc1Nc1cccc(-c2ncn(C)n2)c1OC] |
| 9 RRR | 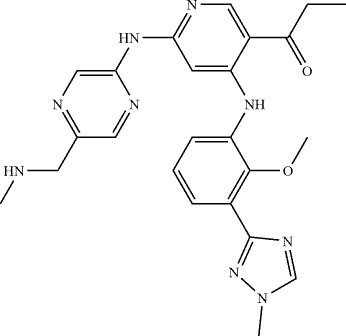 [SMILES: CCC(=O)c1cnc(Nc2cnc(CNC)cn2)cc1Nc1cccc(-c2ncn(C)n2)c1OC] |
| 9 SSS | 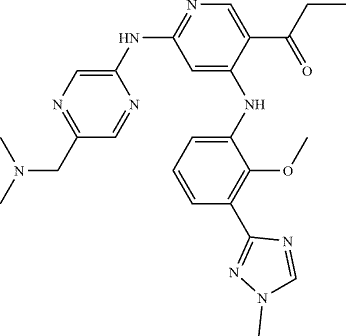 [SMILES: CCC(=O)c1cnc(Nc2cnc(CN(C)C)cn2)cc1Nc1cccc(-c2ncn(C)n2)c1OC] |
| 9 TTT | 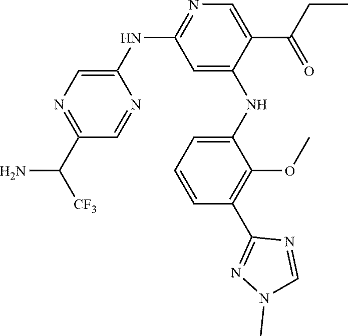 [SMILES: CCC(=O)c1cnc(Nc2cnc(C(N)C(F)(F)F)cn2)cc1Nc1cccc(-c2ncn(C)n2)c1OC] |
| 9 UUU | 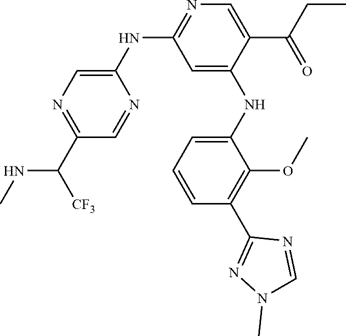 [SMILES: CCC(=O)c1cnc(Nc2cnc(C(NC)C(F)(F)F)cn2)cc1Nc1cccc(-c2ncn(C)n2)c1OC] |
| 9 VVV | 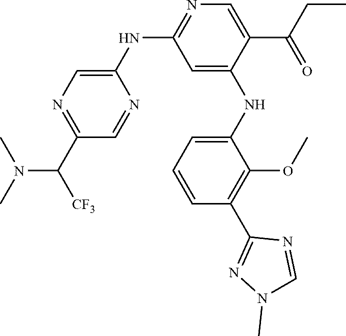 [SMILES: CCC(=O)c1cnc(Nc2cnc(C(N(C)C)C(F)(F)F)cn2)cc1Nc1cccc(-c2ncn(C)n2)c1OC] |
| 9 WWW | 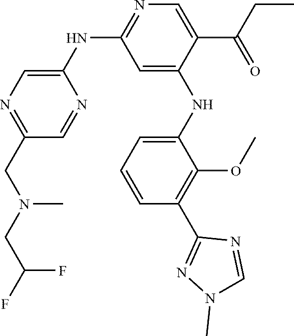 [SMILES: CCC(=O)c1cnc(Nc2cnc(CN(C)CC(F)F)cn2)cc1Nc1cccc(-c2ncn(C)n2)c1OC] |
| 9 XXX | 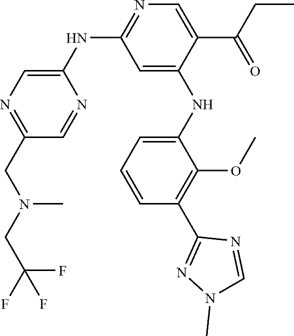 [SMILES: CCC(=O)c1cnc(Nc2cnc(CN(C)CC(F)(F)F)cn2)cc1Nc1cccc(-c2ncn(C)n2)c1OC] |
| 9 YYY | 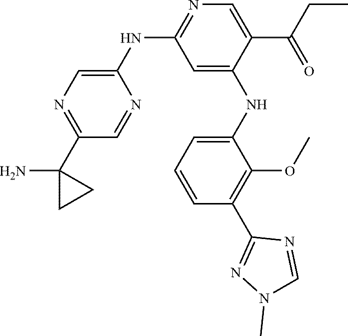 [SMILES: CCC(=O)c1cnc(Nc2cnc(C3(N)CC3)cn2)cc1Nc1cccc(-c2ncn(C)n2)c1OC] |
| 9 ZZZ | 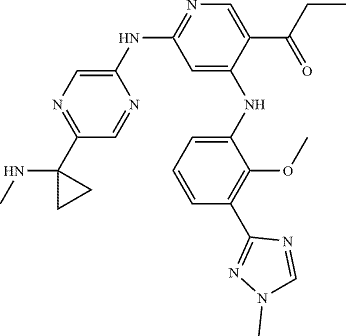 [SMILES: CCC(=O)c1cnc(Nc2cnc(C3(NC)CC3)cn2)cc1Nc1cccc(-c2ncn(C)n2)c1OC] |
| 9 AAAA | 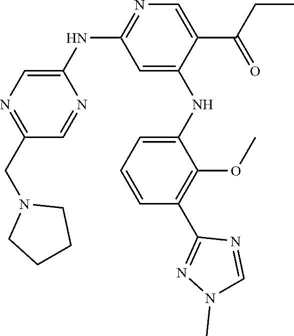 [SMILES: CCC(=O)c1cnc(Nc2cnc(CN3CCCC3)cn2)cc1Nc1cccc(-c2ncn(C)n2)c1OC] |
| 9BBBB | 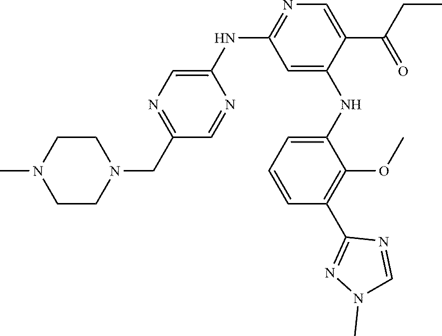 [SMILES: CCC(=O)c1cnc(Nc2cnc(CN3CCN(C)CC3)cn2)cc1Nc1cccc(-c2ncn(C)n2)c1OC] |
| 9CCCC | 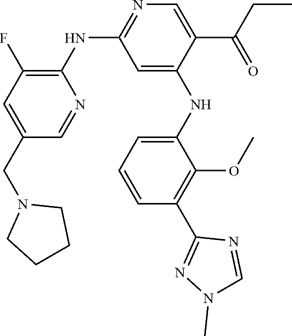 [SMILES: CCC(=O)c1cnc(Nc2ncc(CN3CCCC3)cc2F)cc1Nc1cccc(-c2ncn(C)n2)c1OC] |
| 9 DDDD | 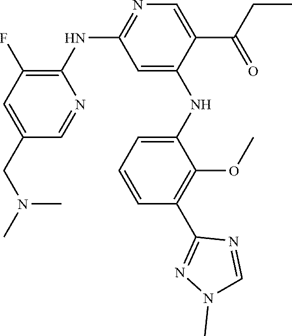 [SMILES: CCC(=O)c1cnc(Nc2ncc(CN(C)C)cc2F)cc1Nc1cccc(-c2ncn(C)n2)c1OC] |
| 9 EEEE | 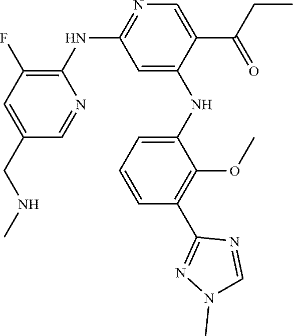 [SMILES: CCC(=O)c1cnc(Nc2ncc(CNC)cc2F)cc1Nc1cccc(-c2ncn(C)n2)c1OC] |
| 9 FFFF | 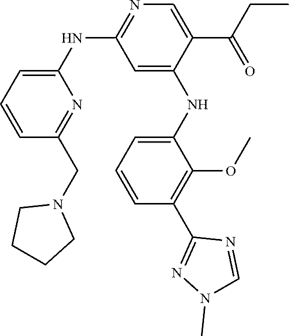 [SMILES: CCC(=O)c1cnc(Nc2cccc(CN3CCCC3)n2)cc1Nc1cccc(-c2ncn(C)n2)c1OC] |
| 9 GGGG | 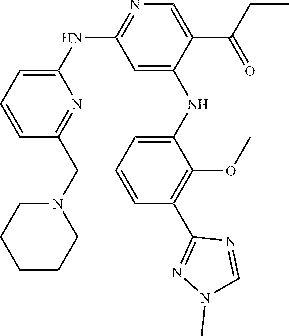 [SMILES: CCC(=O)c1cnc(Nc2cccc(CN3CCCCC3)n2)cc1Nc1cccc(-c2ncn(C)n2)c1OC] |
| 9 HHHH | 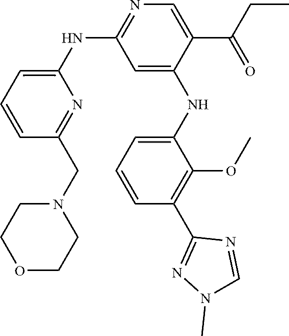 [SMILES: CCC(=O)c1cnc(Nc2cccc(CN3CCOCC3)n2)cc1Nc1cccc(-c2ncn(C)n2)c1OC] |
| 9 IIII | 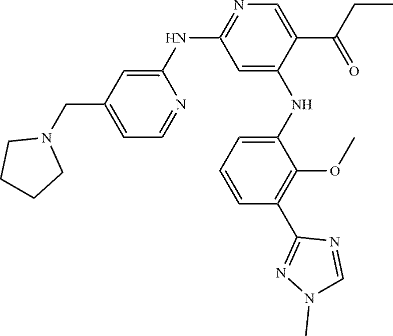 [SMILES: CCC(=O)c1cnc(Nc2cc(CN3CCCC3)ccn2)cc1Nc1cccc(-c2ncn(C)n2)c1OC] |
| 9 JJJJ | 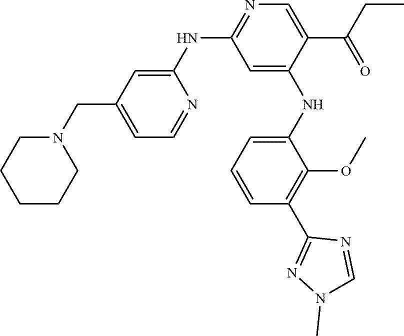 [SMILES: CCC(=O)c1cnc(Nc2cc(CN3CCCCC3)ccn2)cc1Nc1cccc(-c2ncn(C)n2)c1OC] |
| 9 KKKK | 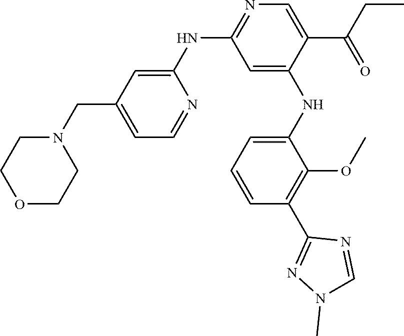 [SMILES: CCC(=O)c1cnc(Nc2cc(CN3CCOCC3)ccn2)cc1Nc1cccc(-c2ncn(C)n2)c1OC] |
| 9 LLLL | 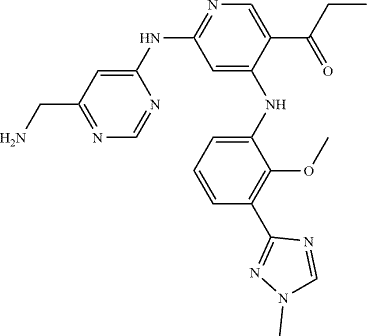 [SMILES: CCC(=O)c1cnc(Nc2cc(CN)ncn2)cc1Nc1cccc(-c2ncn(C)n2)c1OC] |
| 9 MMMM | 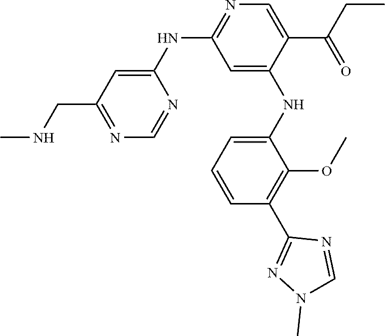 [SMILES: CCC(=O)c1cnc(Nc2cc(CNC)ncn2)cc1Nc1cccc(-c2ncn(C)n2)c1OC] |
| 9 NNNN | 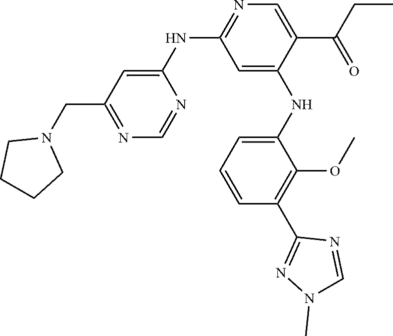 [SMILES: CCC(=O)c1cnc(Nc2cc(CN3CCCC3)ncn2)cc1Nc1cccc(-c2ncn(C)n2)c1OC] |
| 9OOOO | 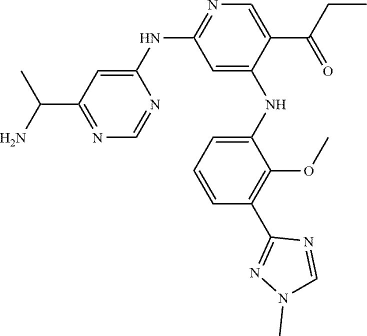 [SMILES: CCC(=O)c1cnc(Nc2cc(C(C)N)ncn2)cc1Nc1cccc(-c2ncn(C)n2)c1OC] |
| 9 PPPP | 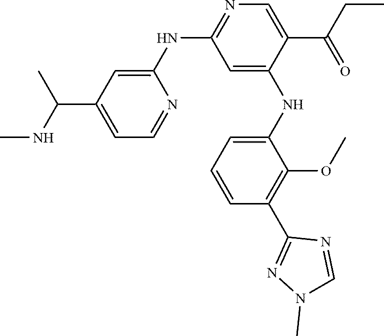 [SMILES: CCC(=O)c1cnc(Nc2cc(C(C)NC)ccn2)cc1Nc1cccc(-c2ncn(C)n2)c1OC] |
| 9 QQQQ | 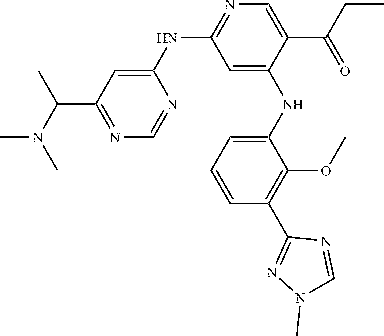 [SMILES: CCC(=O)c1cnc(Nc2cc(C(C)N(C)C)ncn2)cc1Nc1cccc(-c2ncn(C)n2)c1OC] |
| 9 RRRR | 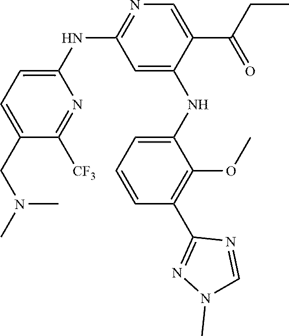 [SMILES: CCC(=O)c1cnc(Nc2ccc(CN(C)C)c(C(F)(F)F)n2)cc1Nc1cccc(-c2ncn(C)n2)c1OC] |
| 9 SSSS | 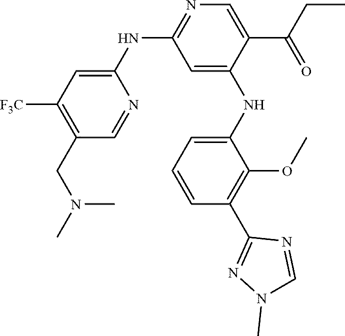 [SMILES: CCC(=O)c1cnc(Nc2cc(C(F)(F)F)c(CN(C)C)cn2)cc1Nc1cccc(-c2ncn(C)n2)c1OC] |
| 9 TTTT | 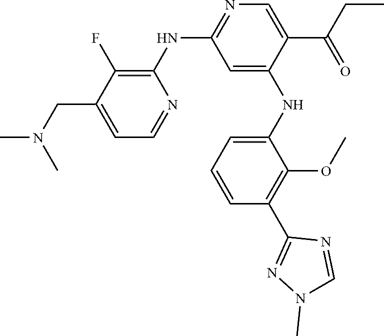 [SMILES: CCC(=O)c1cnc(Nc2nccc(CN(C)C)c2F)cc1Nc1cccc(-c2ncn(C)n2)c1OC] |
| 9 UUUU | 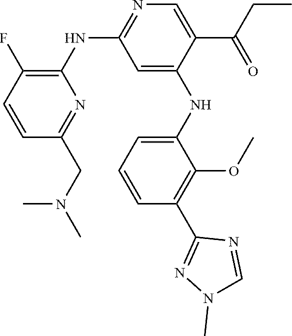 [SMILES: CCC(=O)c1cnc(Nc2nc(CN(C)C)ccc2F)cc1Nc1cccc(-c2ncn(C)n2)c1OC] |
| 10A | 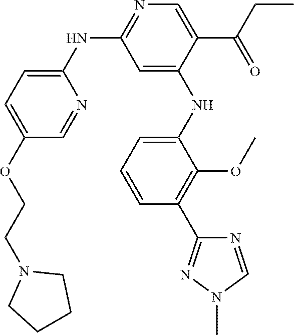 [SMILES: CCC(=O)c1cnc(Nc2ccc(OCCN3CCCC3)cn2)cc1Nc1cccc(-c2ncn(C)n2)c1OC] |
| 11A | 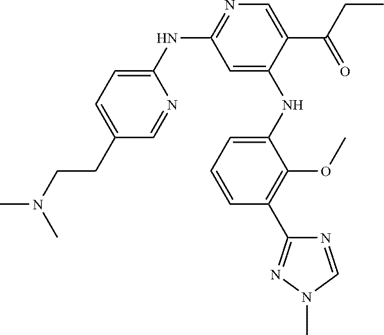 [SMILES: CCC(=O)c1cnc(Nc2ccc(CCN(C)C)cn2)cc1Nc1cccc(-c2ncn(C)n2)c1OC] |
| 11B | 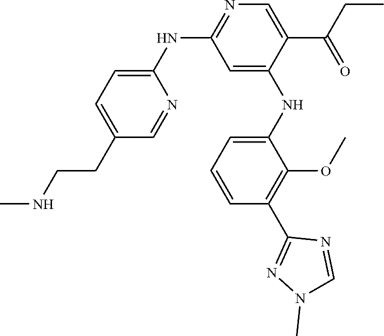 [SMILES: CCC(=O)c1cnc(Nc2ccc(CCNC)cn2)cc1Nc1cccc(-c2ncn(C)n2)c1OC] |
| 11C | 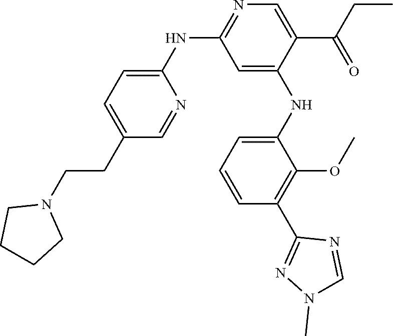 [SMILES: CCC(=O)c1cnc(Nc2ccc(CCN3CCCC3)cn2)cc1Nc1cccc(-c2ncn(C)n2)c1OC] |
| 11D | 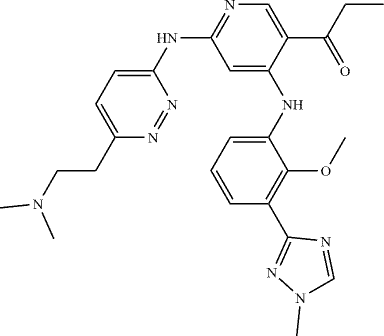 [SMILES: CCC(=O)c1cnc(Nc2ccc(CCN(C)C)nn2)cc1Nc1cccc(-c2ncn(C)n2)c1OC] |
| 11E | 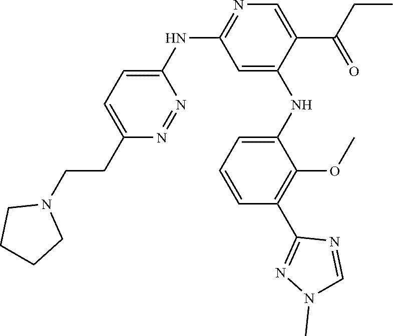 [SMILES: CCC(=O)c1cnc(Nc2ccc(CCN3CCCC3)nn2)cc1Nc1cccc(-c2ncn(C)n2)c1OC] |
| 11F | 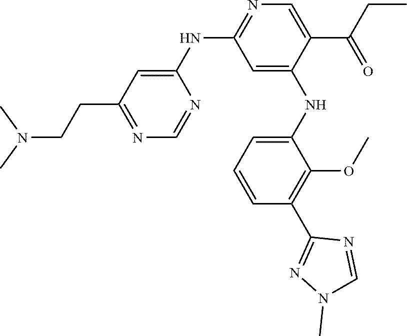 [SMILES: CCC(=O)c1cnc(Nc2cc(CCN(C)C)ncn2)cc1Nc1cccc(-c2ncn(C)n2)c1OC] |
| 11G | 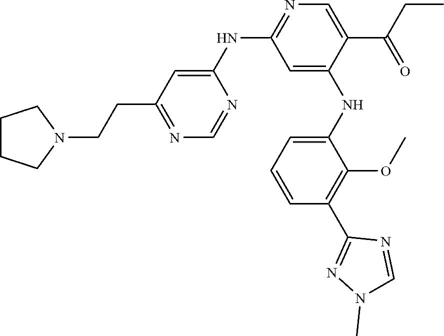 [SMILES: CCC(=O)c1cnc(Nc2cc(CCN3CCCC3)ncn2)cc1Nc1cccc(-c2ncn(C)n2)c1OC] |
| 11H | 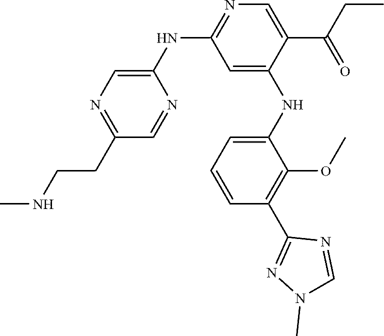 [SMILES: CCC(=O)c1cnc(Nc2cnc(CCNC)cn2)cc1Nc1cccc(-c2ncn(C)n2)c1OC] |
| 11I | 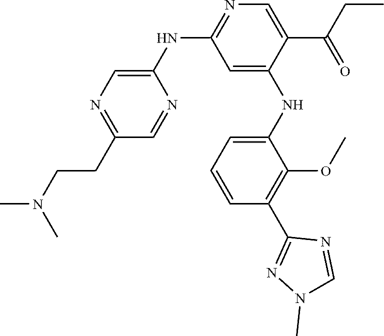 [SMILES: CCC(=O)c1cnc(Nc2cnc(CCN(C)C)cn2)cc1Nc1cccc(-c2ncn(C)n2)c1OC] |
| 11J | 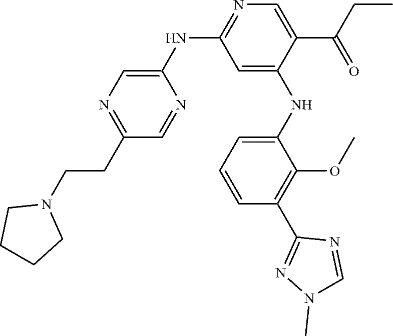 [SMILES: CCC(=O)c1cnc(Nc2cnc(CCN3CCCC3)cn2)cc1Nc1cccc(-c2ncn(C)n2)c1OC] |
| 12A | 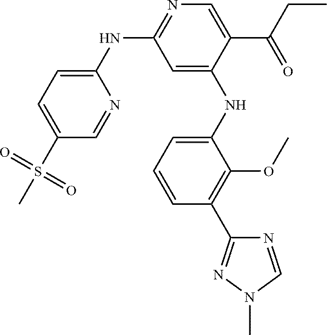 [SMILES: CCC(=O)c1cnc(Nc2ccc(S(C)(=O)=O)cn2)cc1Nc1cccc(-c2ncn(C)n2)c1OC] |
| 13A | 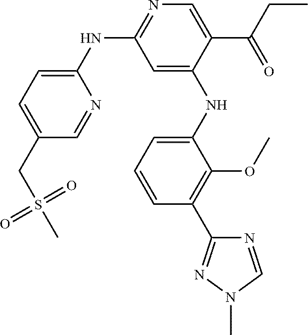 [SMILES: CCC(=O)c1cnc(Nc2ccc(CS(C)(=O)=O)cn2)cc1Nc1cccc(-c2ncn(C)n2)c1OC] |
| 13B | 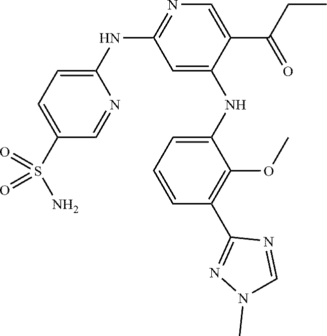 [SMILES: CCC(=O)c1cnc(Nc2ccc(S(N)(=O)=O)cn2)cc1Nc1cccc(-c2ncn(C)n2)c1OC] |
| 13C | 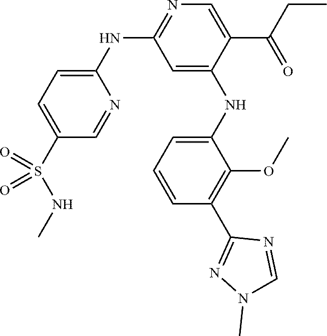 [SMILES: CCC(=O)c1cnc(Nc2ccc(S(=O)(=O)NC)cn2)cc1Nc1cccc(-c2ncn(C)n2)c1OC] |
| 13D | 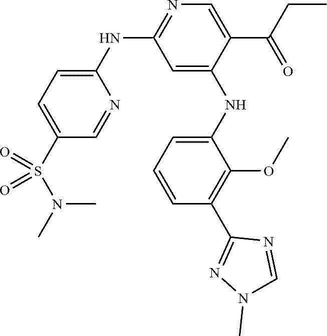 [SMILES: CCC(=O)c1cnc(Nc2ccc(S(=O)(=O)N(C)C)cn2)cc1Nc1cccc(-c2ncn(C)n2)c1OC] |
| 13E | 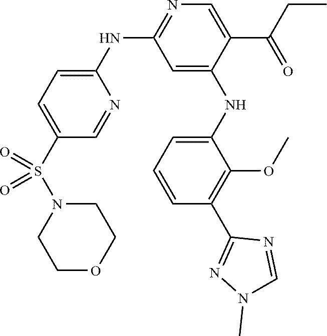 [SMILES: CCC(=O)c1cnc(Nc2ccc(S(=O)(=O)N3CCOCC3)cn2)cc1Nc1cccc(-c2ncn(C)n2)c1OC] |
| 13F | 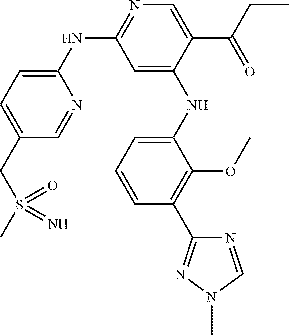 [SMILES: CCC(=O)c1cnc(Nc2ccc(CS(C)(=N)=O)cn2)cc1Nc1cccc(-c2ncn(C)n2)c1OC] |
| 13G | 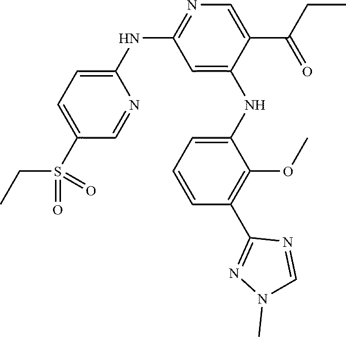 [SMILES: CCC(=O)c1cnc(Nc2ccc(S(=O)(=O)CC)cn2)cc1Nc1cccc(-c2ncn(C)n2)c1OC] |
| 13H | 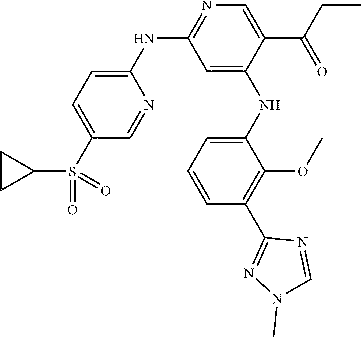 [SMILES: CCC(=O)c1cnc(Nc2ccc(S(=O)(=O)C3CC3)cn2)cc1Nc1cccc(-c2ncn(C)n2)c1OC] |
| 13I | 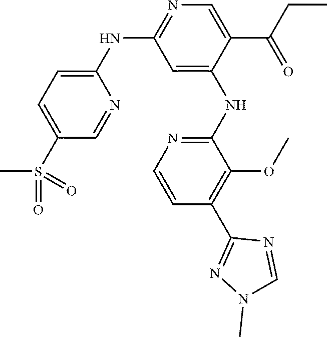 [SMILES: CCC(=O)c1cnc(Nc2ccc(S(C)(=O)=O)cn2)cc1Nc1nccc(-c2ncn(C)n2)c1OC] |
| 13J | 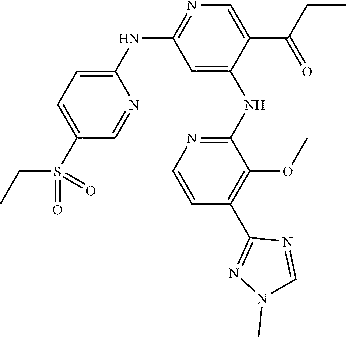 [SMILES: CCC(=O)c1cnc(Nc2ccc(S(=O)(=O)CC)cn2)cc1Nc1nccc(-c2ncn(C)n2)c1OC] |
| 13K | 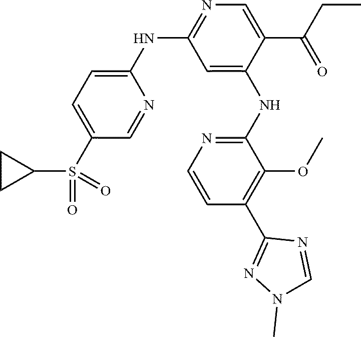 [SMILES: CCC(=O)c1cnc(Nc2ccc(S(=O)(=O)C3CC3)cn2)cc1Nc1nccc(-c2ncn(C)n2)c1OC] |
| 13L | 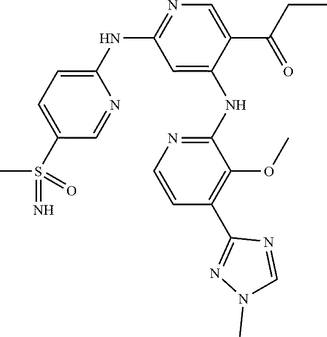 [SMILES: CCC(=O)c1cnc(Nc2ccc(S(C)(=N)=O)cn2)cc1Nc1nccc(-c2ncn(C)n2)c1OC] |
| 13M | 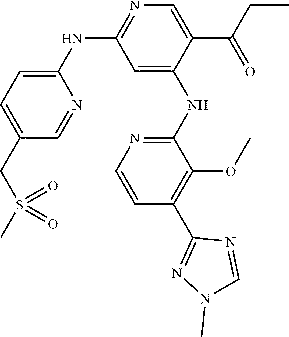 [SMILES: CCC(=O)c1cnc(Nc2ccc(CS(C)(=O)=O)cn2)cc1Nc1nccc(-c2ncn(C)n2)c1OC] |
| 13N | 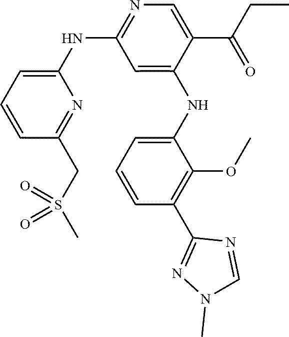 [SMILES: CCC(=O)c1cnc(Nc2cccc(CS(C)(=O)=O)n2)cc1Nc1cccc(-c2ncn(C)n2)c1OC] |
| 13O | 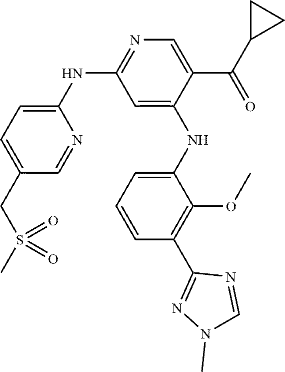 [SMILES: COc1c(Nc2cc(Nc3ccc(CS(C)(=O)=O)cn3)ncc2C(=O)C2CC2)cccc1-c1ncn(C)n1] |
| 13P | 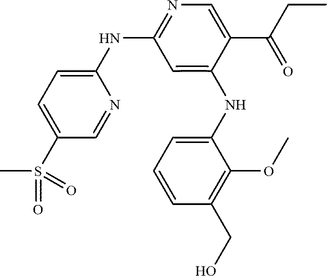 [SMILES: CCC(=O)c1cnc(Nc2ccc(S(C)(=O)=O)cn2)cc1Nc1cccc(CO)c1OC] |
| 13Q | 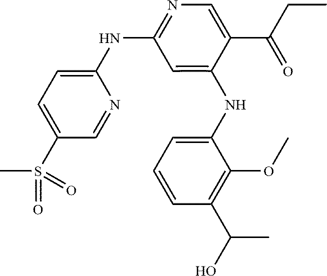 [SMILES: CCC(=O)c1cnc(Nc2ccc(S(C)(=O)=O)cn2)cc1Nc1cccc(C(C)O)c1OC] |
| 13R | 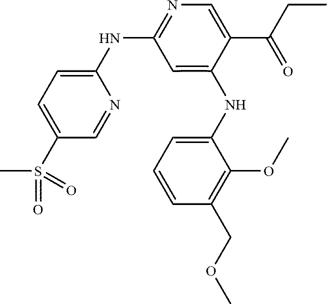 [SMILES: CCC(=O)c1cnc(Nc2ccc(S(C)(=O)=O)cn2)cc1Nc1cccc(COC)c1OC] |
| 13D | 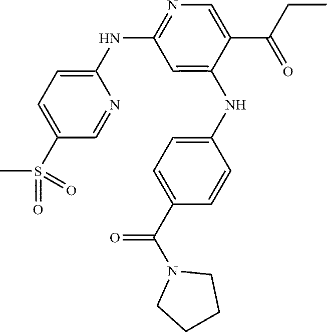 [SMILES: CCC(=O)c1cnc(Nc2ccc(S(C)(=O)=O)cn2)cc1Nc1ccc(C(=O)N2CCCC2)cc1] |
| 13T | 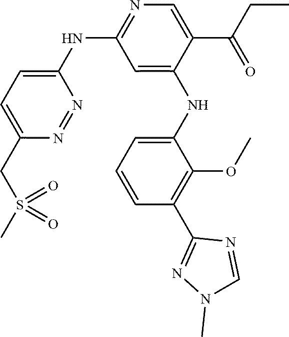 [SMILES: CCC(=O)c1cnc(Nc2ccc(CS(C)(=O)=O)nn2)cc1Nc1cccc(-c2ncn(C)n2)c1OC] |
| 13U | 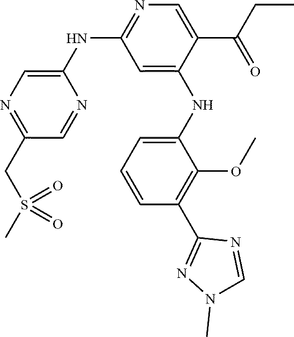 [SMILES: CCC(=O)c1cnc(Nc2cnc(CS(C)(=O)=O)cn2)cc1Nc1cccc(-c2ncn(C)n2)c1OC] |
| 13V | 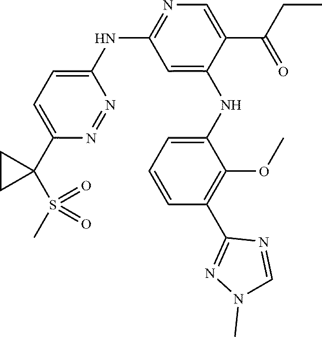 [SMILES: CCC(=O)c1cnc(Nc2ccc(C3(S(C)(=O)=O)CC3)nn2)cc1Nc1cccc(-c2ncn(C)n2)c1OC] |
| 13W | 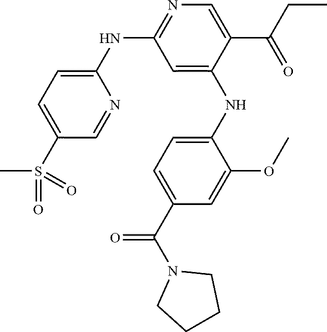 [SMILES: CCC(=O)c1cnc(Nc2ccc(S(C)(=O)=O)cn2)cc1Nc1ccc(C(=O)N2CCCC2)cc1OC] |
| 13X | 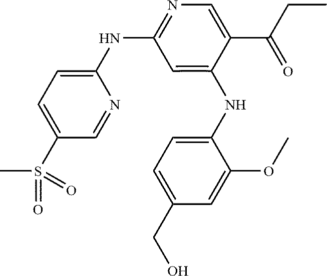 [SMILES: CCC(=O)c1cnc(Nc2ccc(S(C)(=O)=O)cn2)cc1Nc1ccc(CO)cc1OC] |
| 13Y | 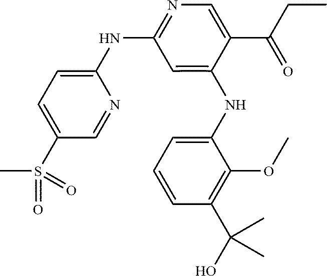 [SMILES: CCC(=O)c1cnc(Nc2ccc(S(C)(=O)=O)cn2)cc1Nc1cccc(C(C)(C)O)c1OC] |
| 13Z | 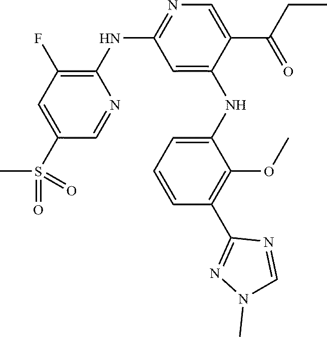 [SMILES: CCC(=O)c1cnc(Nc2ncc(S(C)(=O)=O)cc2F)cc1Nc1cccc(-c2ncn(C)n2)c1OC] |
| 13 AA | 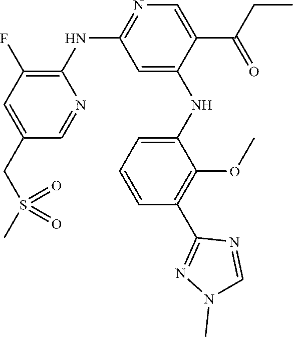 [SMILES: CCC(=O)c1cnc(Nc2ncc(CS(C)(=O)=O)cc2F)cc1Nc1cccc(-c2ncn(C)n2)c1OC] |
| 14A | 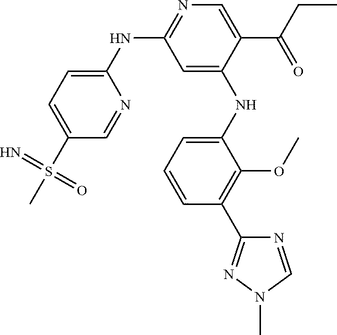 [SMILES: CCC(=O)c1cnc(Nc2ccc(S(C)(=N)=O)cn2)cc1Nc1cccc(-c2ncn(C)n2)c1OC] |
| 15A | 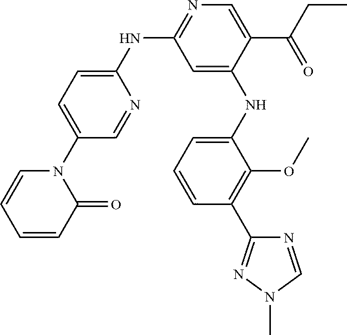 [SMILES: CCC(=O)c1cnc(Nc2ccc(-n3ccccc3=O)cn2)cc1Nc1cccc(-c2ncn(C)n2)c1OC] |
| 15B | 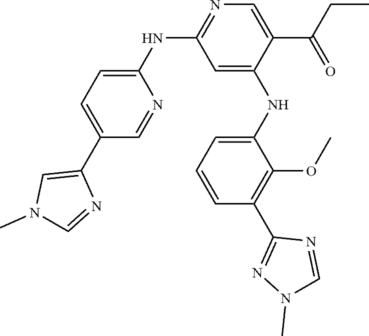 [SMILES: CCC(=O)c1cnc(Nc2ccc(-c3cn(C)cn3)cn2)cc1Nc1cccc(-c2ncn(C)n2)c1OC] |
| 15C | 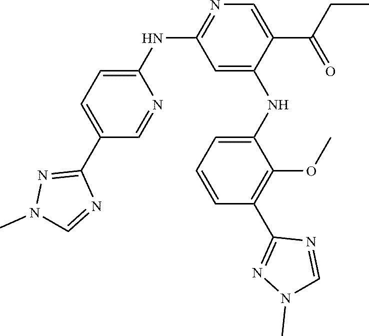 [SMILES: CCC(=O)c1cnc(Nc2ccc(-c3ncn(C)n3)cn2)cc1Nc1cccc(-c2ncn(C)n2)c1OC] |
| 15D | 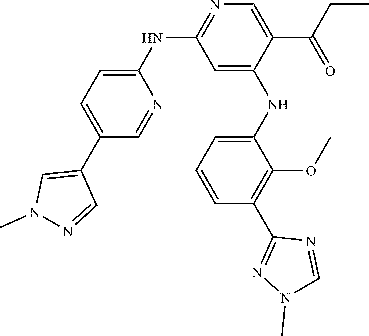 [SMILES: CCC(=O)c1cnc(Nc2ccc(-c3cnn(C)c3)cn2)cc1Nc1cccc(-c2ncn(C)n2)c1OC] |
| 15E | 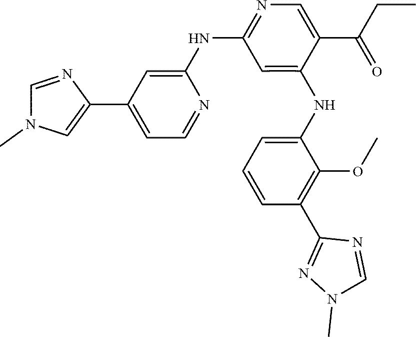 [SMILES: CCC(=O)c1cnc(Nc2cc(-c3cn(C)cn3)ccn2)cc1Nc1cccc(-c2ncn(C)n2)c1OC] |
| 15F | 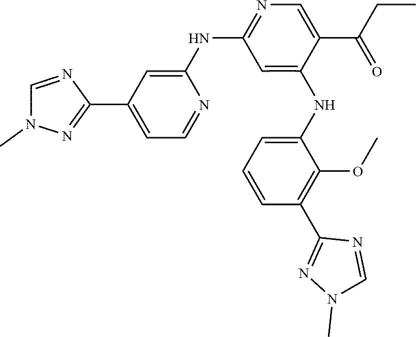 [SMILES: CCC(=O)c1cnc(Nc2cc(-c3ncn(C)n3)ccn2)cc1Nc1cccc(-c2ncn(C)n2)c1OC] |
| 15G | 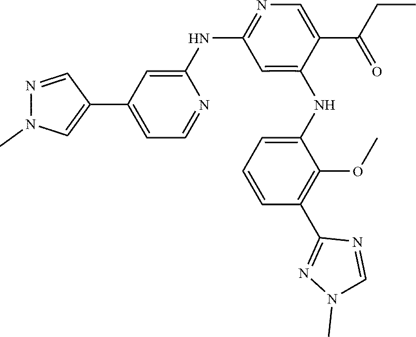 [SMILES: CCC(=O)c1cnc(Nc2cc(-c3cnn(C)c3)ccn2)cc1Nc1cccc(-c2ncn(C)n2)c1OC] |
| 15H | 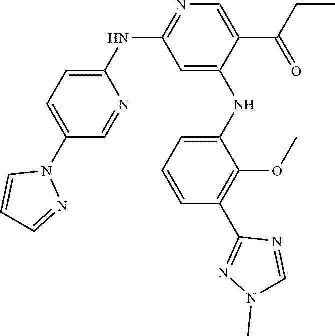 [SMILES: CCC(=O)c1cnc(Nc2ccc(-n3cccn3)cn2)cc1Nc1cccc(-c2ncn(C)n2)c1OC] |
| 15I | 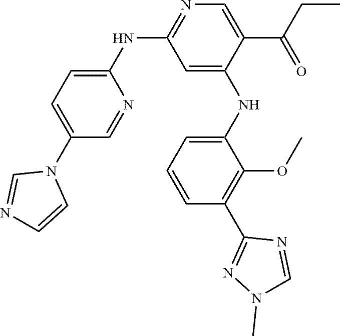 [SMILES: CCC(=O)c1cnc(Nc2ccc(-n3ccnc3)cn2)cc1Nc1cccc(-c2ncn(C)n2)c1OC] |
| 15J | 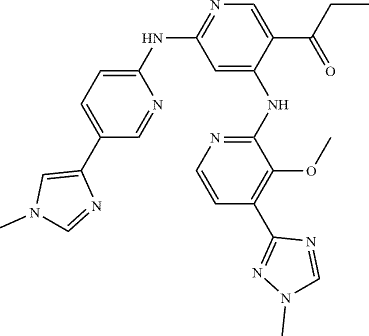 [SMILES: CCC(=O)c1cnc(Nc2ccc(-c3cn(C)cn3)cn2)cc1Nc1nccc(-c2ncn(C)n2)c1OC] |
| 15K | 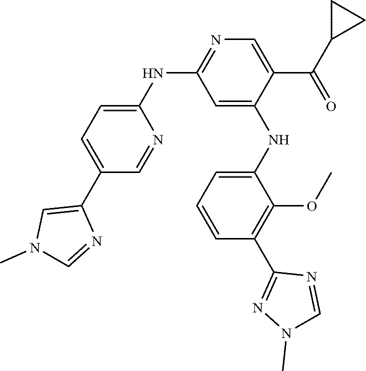 [SMILES: COc1c(Nc2cc(Nc3ccc(-c4cn(C)cn4)cn3)ncc2C(=O)C2CC2)cccc1-c1ncn(C)n1] |
| 15L | 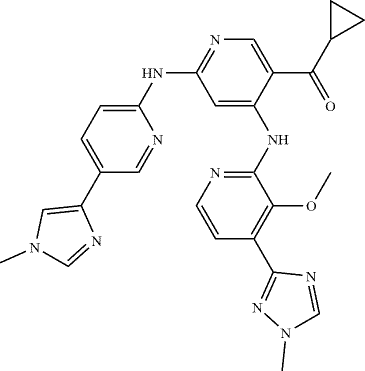 [SMILES: COc1c(-c2ncn(C)n2)ccnc1Nc1cc(Nc2ccc(-c3cn(C)cn3)cn2)ncc1C(=O)C1CC1] |
| 15M | 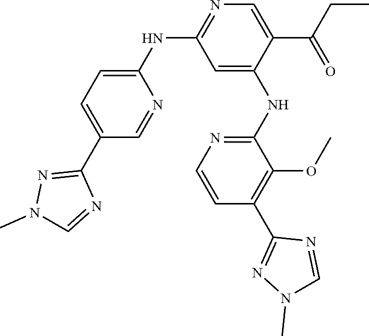 [SMILES: CCC(=O)c1cnc(Nc2ccc(-c3ncn(C)n3)cn2)cc1Nc1nccc(-c2ncn(C)n2)c1OC] |
| 15N | 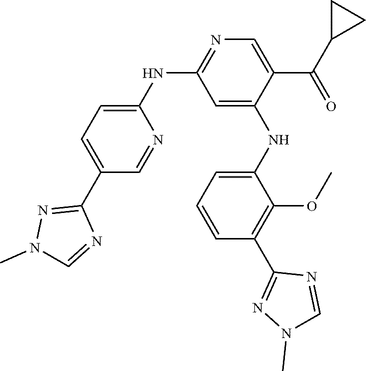 [SMILES: COc1c(Nc2cc(Nc3ccc(-c4ncn(C)n4)cn3)ncc2C(=O)C2CC2)cccc1-c1ncn(C)n1] |
| 15O | 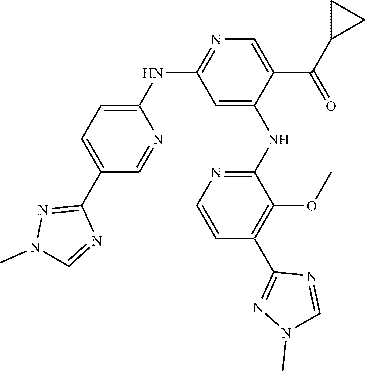 [SMILES: COc1c(-c2ncn(C)n2)ccnc1Nc1cc(Nc2ccc(-c3ncn(C)n3)cn2)ncc1C(=O)C1CC1] |
| 15P | 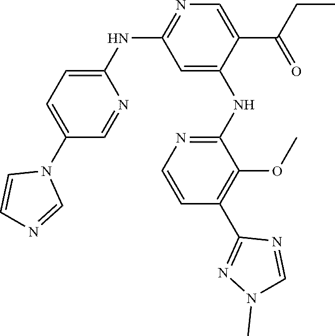 [SMILES: CCC(=O)c1cnc(Nc2ccc(-n3ccnc3)cn2)cc1Nc1nccc(-c2ncn(C)n2)c1OC] |
| 15Q | 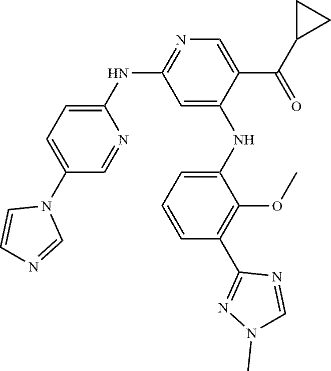 [SMILES: COc1c(Nc2cc(Nc3ccc(-n4ccnc4)cn3)ncc2C(=O)C2CC2)cccc1-c1ncn(C)n1] |
| 15R | 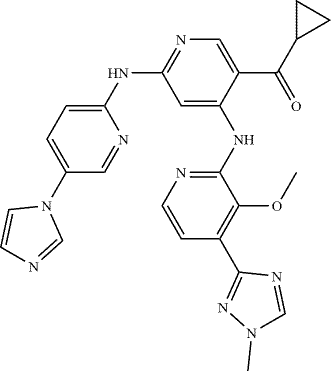 [SMILES: COc1c(-c2ncn(C)n2)ccnc1Nc1cc(Nc2ccc(-n3ccnc3)cn2)ncc1C(=O)C1CC1] |
| 15S | 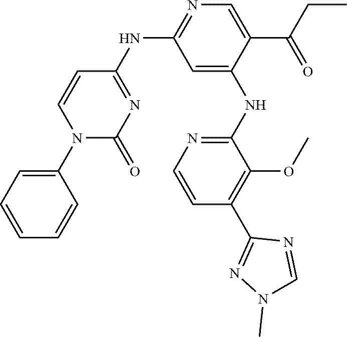 [SMILES: CCC(=O)c1cnc(Nc2ccn(-c3ccccc3)c(=O)n2)cc1Nc1nccc(-c2ncn(C)n2)c1OC] |
| 15T | 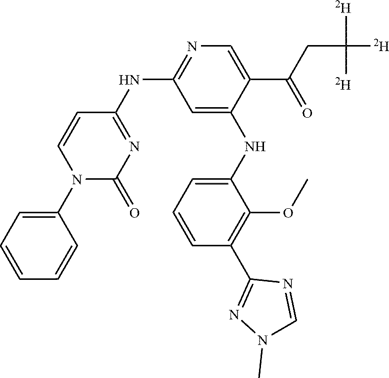 [SMILES: [2H]C([2H])([2H])CC(=O)c1cnc(Nc2ccn(-c3ccccc3)c(=O)n2)cc1Nc1cccc(-c2ncn(C)n2)c1OC] |
| 15U | 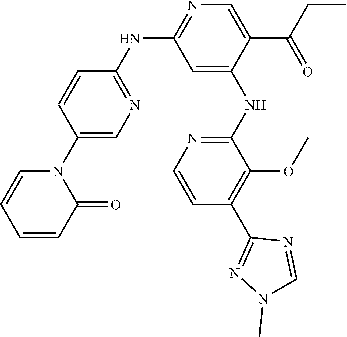 [SMILES: CCC(=O)c1cnc(Nc2ccc(-n3ccccc3=O)cn2)cc1Nc1nccc(-c2ncn(C)n2)c1OC] |
| 15V | 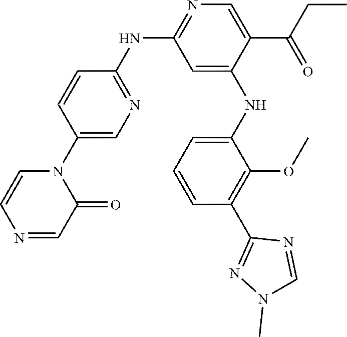 [SMILES: CCC(=O)c1cnc(Nc2ccc(-n3ccncc3=O)cn2)cc1Nc1cccc(-c2ncn(C)n2)c1OC] |
| 15W | 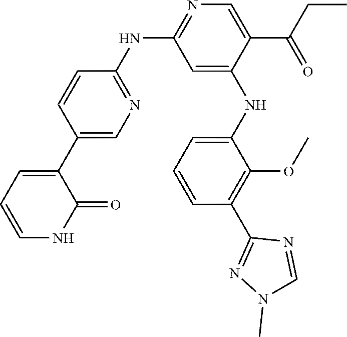 [SMILES: CCC(=O)c1cnc(Nc2ccc(-c3ccc[nH]c3=O)cn2)cc1Nc1cccc(-c2ncn(C)n2)c1OC] |
| 15X | 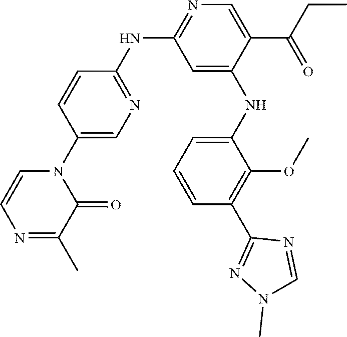 [SMILES: CCC(=O)c1cnc(Nc2ccc(-n3ccnc(C)c3=O)cn2)cc1Nc1cccc(-c2ncn(C)n2)c1OC] |
| 15Y | 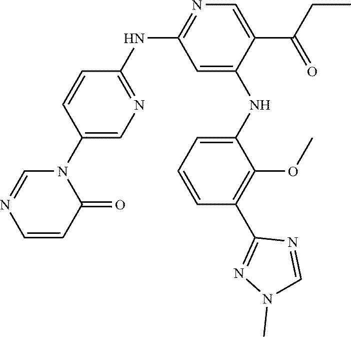 [SMILES: CCC(=O)c1cnc(Nc2ccc(-n3cnccc3=O)cn2)cc1Nc1cccc(-c2ncn(C)n2)c1OC] |
| 15Z | 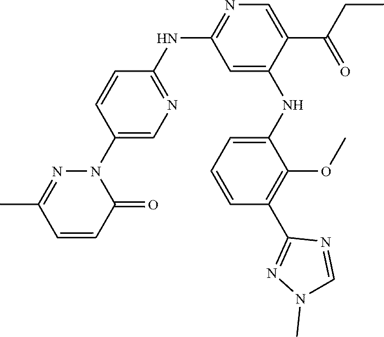 [SMILES: CCC(=O)c1cnc(Nc2ccc(-n3nc(C)ccc3=O)cn2)cc1Nc1cccc(-c2ncn(C)n2)c1OC] |
| 15AA | 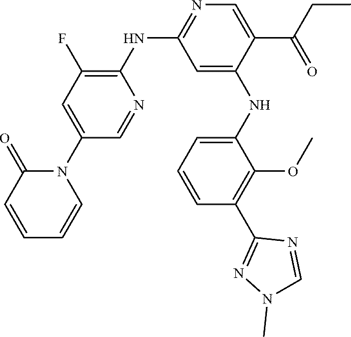 [SMILES: CCC(=O)c1cnc(Nc2ncc(-n3ccccc3=O)cc2F)cc1Nc1cccc(-c2ncn(C)n2)c1OC] |
| 15BB | 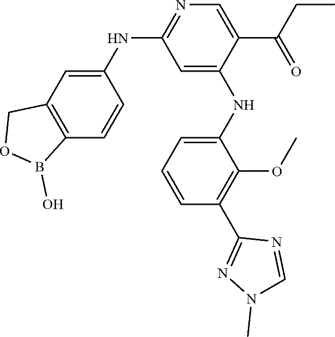 [SMILES: CCC(=O)c1cnc(Nc2ccc3c(c2)COB3O)cc1Nc1cccc(-c2ncn(C)n2)c1OC] |
| 15CC | 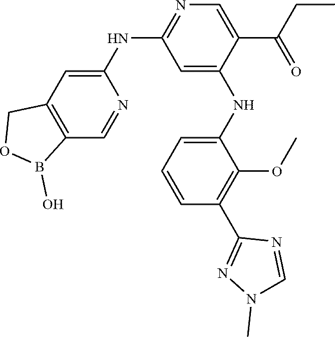 [SMILES: CCC(=O)c1cnc(Nc2cc3c(cn2)B(O)OC3)cc1Nc1cccc(-c2ncn(C)n2)c1OC] |
| 15DD | 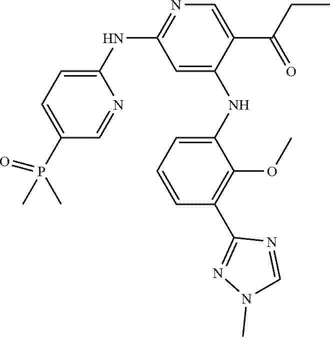 [SMILES: CCC(=O)c1cnc(Nc2ccc(P(C)(C)=O)cn2)cc1Nc1cccc(-c2ncn(C)n2)c1OC] |
| 16A | 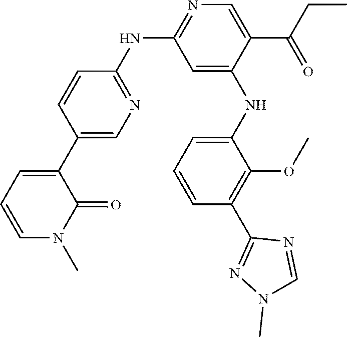 [SMILES: CCC(=O)c1cnc(Nc2ccc(-c3cccn(C)c3=O)cn2)cc1Nc1cccc(-c2ncn(C)n2)c1OC] |
| 17A | 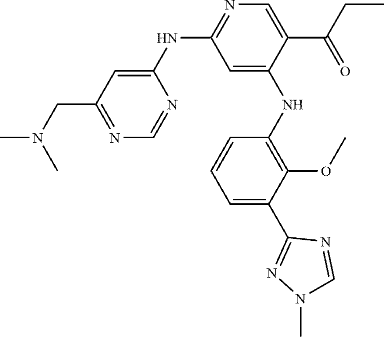 [SMILES: CCC(=O)c1cnc(Nc2cc(CN(C)C)ncn2)cc1Nc1cccc(-c2ncn(C)n2)c1OC] |
| 18A | 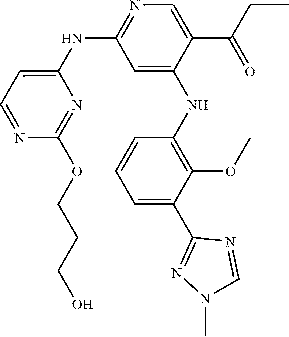 [SMILES: CCC(=O)c1cnc(Nc2ccnc(OCCCO)n2)cc1Nc1cccc(-c2ncn(C)n2)c1OC] |
| 18B | 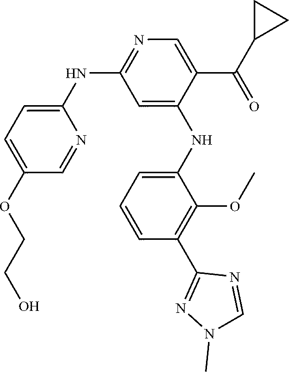 [SMILES: COc1c(Nc2cc(Nc3ccc(OCCO)cn3)ncc2C(=O)C2CC2)cccc1-c1ncn(C)n1] |
| 18C | 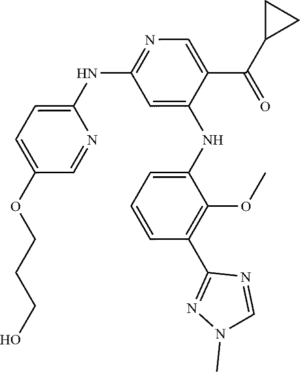 [SMILES: COc1c(Nc2cc(Nc3ccc(OCCCO)cn3)ncc2C(=O)C2CC2)cccc1-c1ncn(C)n1] |
| 18D | 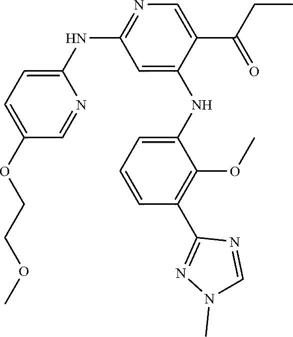 [SMILES: CCC(=O)c1cnc(Nc2ccc(OCCOC)cn2)cc1Nc1cccc(-c2ncn(C)n2)c1OC] |
| 18E | 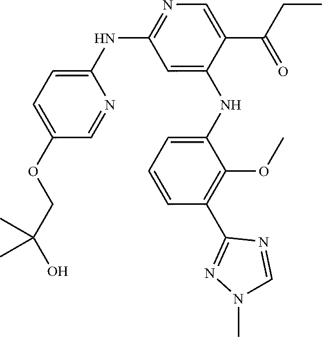 [SMILES: CCC(=O)c1cnc(Nc2ccc(OCC(C)(C)O)cn2)cc1Nc1cccc(-c2ncn(C)n2)c1OC] |
| 18F | 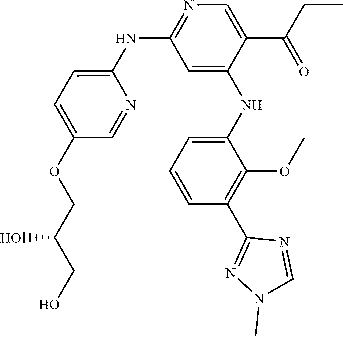 [SMILES: CCC(=O)c1cnc(Nc2ccc(OC[C@@H](O)CO)cn2)cc1Nc1cccc(-c2ncn(C)n2)c1OC] |
| 18G | 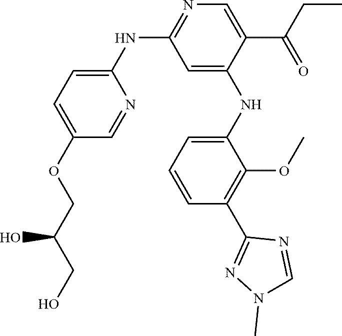 [SMILES: CCC(=O)c1cnc(Nc2ccc(OC[C@H](O)CO)cn2)cc1Nc1cccc(-c2ncn(C)n2)c1OC] |
| 18H | 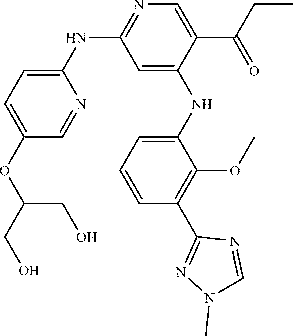 [SMILES: CCC(=O)c1cnc(Nc2ccc(OC(CO)CO)cn2)cc1Nc1cccc(-c2ncn(C)n2)c1OC] |
| 18I | 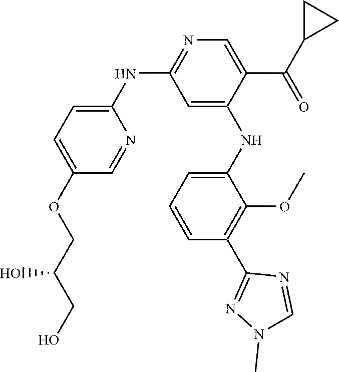 [SMILES: COc1c(Nc2cc(Nc3ccc(OC[C@@H](O)CO)cn3)ncc2C(=O)C2CC2)cccc1-c1ncn(C)n1] |
| 18J | 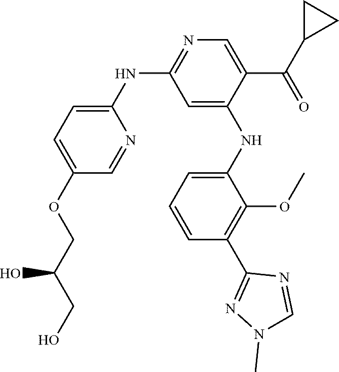 [SMILES: COc1c(Nc2cc(Nc3ccc(OC[C@H](O)CO)cn3)ncc2C(=O)C2CC2)cccc1-c1ncn(C)n1] |
| 18K | 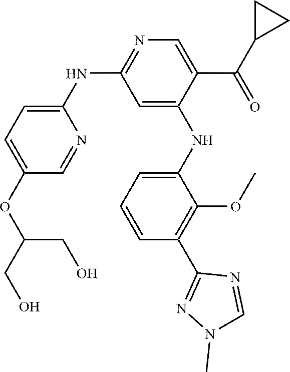 [SMILES: COc1c(Nc2cc(Nc3ccc(OC(CO)CO)cn3)ncc2C(=O)C2CC2)cccc1-c1ncn(C)n1] |
| 18L | 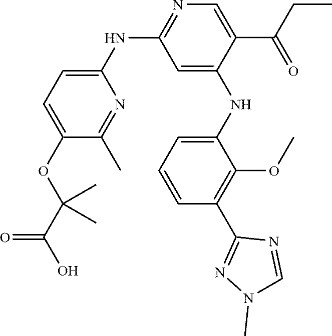 [SMILES: CCC(=O)c1cnc(Nc2ccc(OC(C)(C)C(=O)O)c(C)n2)cc1Nc1cccc(-c2ncn(C)n2)c1OC] |
| 18M | 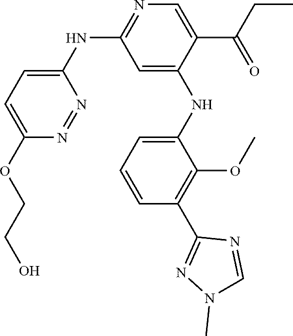 [SMILES: CCC(=O)c1cnc(Nc2ccc(OCCO)nn2)cc1Nc1cccc(-c2ncn(C)n2)c1OC] |
| 18N | 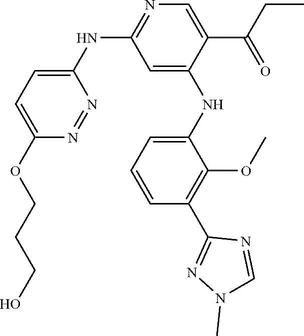 [SMILES: CCC(=O)c1cnc(Nc2ccc(OCCCO)nn2)cc1Nc1cccc(-c2ncn(C)n2)c1OC] |
| 18O | 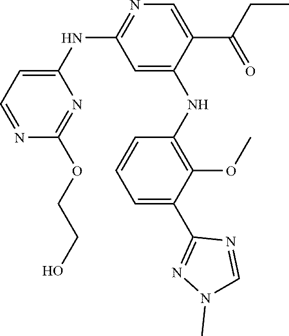 [SMILES: CCC(=O)c1cnc(Nc2ccnc(OCCO)n2)cc1Nc1cccc(-c2ncn(C)n2)c1OC] |
| 18P | 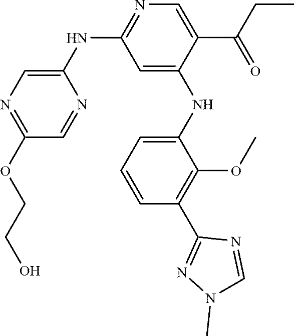 [SMILES: CCC(=O)c1cnc(Nc2cnc(OCCO)cn2)cc1Nc1cccc(-c2ncn(C)n2)c1OC] |
| 18Q | 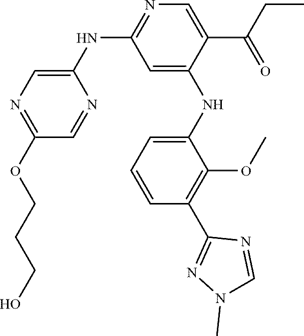 [SMILES: CCC(=O)c1cnc(Nc2cnc(OCCCO)cn2)cc1Nc1cccc(-c2ncn(C)n2)c1OC] |
| 18R | 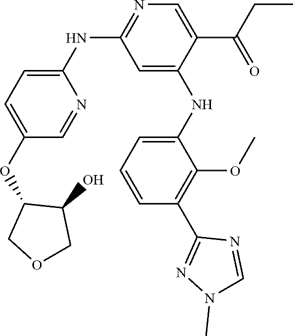 [SMILES: CCC(=O)c1cnc(Nc2ccc(O[C@H]3COC[C@@H]3O)cn2)cc1Nc1cccc(-c2ncn(C)n2)c1OC] |
| 18S | 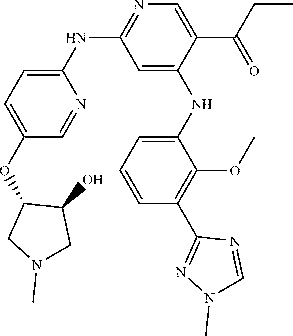 [SMILES: CCC(=O)c1cnc(Nc2ccc(O[C@H]3CN(C)C[C@@H]3O)cn2)cc1Nc1cccc(-c2ncn(C)n2)c1OC] |
| 18T | 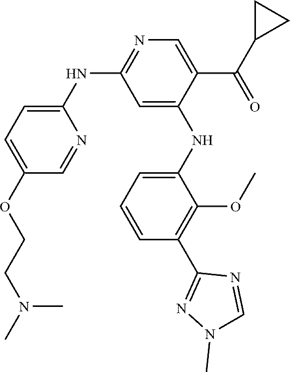 [SMILES: COc1c(Nc2cc(Nc3ccc(OCCN(C)C)cn3)ncc2C(=O)C2CC2)cccc1-c1ncn(C)n1] |
| 18U | 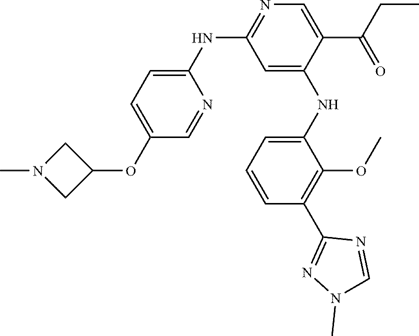 [SMILES: CCC(=O)c1cnc(Nc2ccc(OC3CN(C)C3)cn2)cc1Nc1cccc(-c2ncn(C)n2)c1OC] |
| 18V | 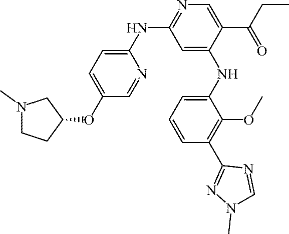 [SMILES: CCC(=O)c1cnc(Nc2ccc(O[C@@H]3CCN(C)C3)cn2)cc1Nc1cccc(-c2ncn(C)n2)c1OC] |
| 18W | 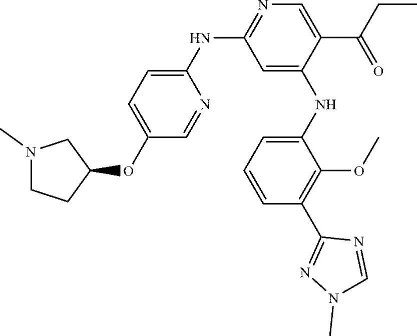 [SMILES: CCC(=O)c1cnc(Nc2ccc(O[C@H]3CCN(C)C3)cn2)cc1Nc1cccc(-c2ncn(C)n2)c1OC] |
| 18X | 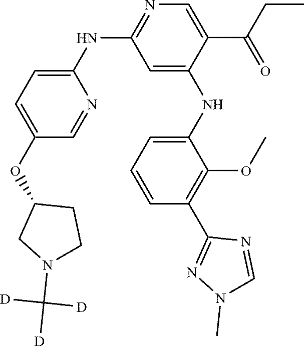 [SMILES: [2H]C([2H])([2H])N1CC[C@@H](Oc2ccc(Nc3cc(Nc4cccc(-c5ncn(C)n5)c4OC)c(C(=O)CC)cn3)nc2)C1] |
| 18Y | 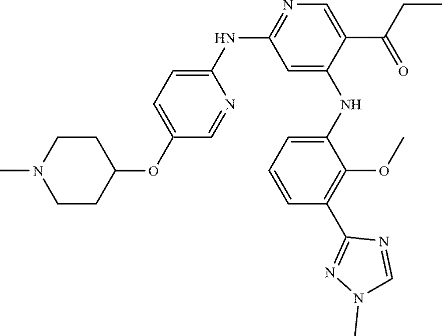 [SMILES: CCC(=O)c1cnc(Nc2ccc(OC3CCN(C)CC3)cn2)cc1Nc1cccc(-c2ncn(C)n2)c1OC] |
| 18Z | 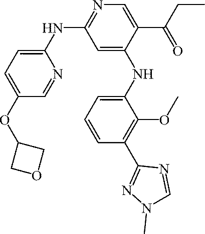 [SMILES: CCC(=O)c1cnc(Nc2ccc(OC3COC3)cn2)cc1Nc1cccc(-c2ncn(C)n2)c1OC] |
| 18AA | 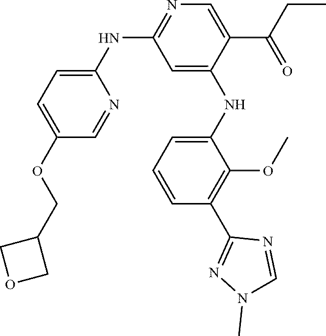 [SMILES: CCC(=O)c1cnc(Nc2ccc(OCC3COC3)cn2)cc1Nc1cccc(-c2ncn(C)n2)c1OC] |
| 18BB | 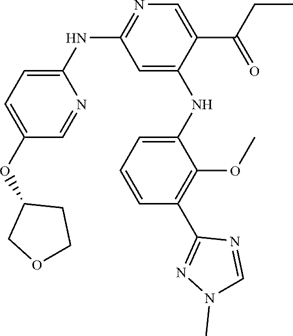 [SMILES: CCC(=O)c1cnc(Nc2ccc(O[C@@H]3CCOC3)cn2)cc1Nc1cccc(-c2ncn(C)n2)c1OC] |
| 18CC | 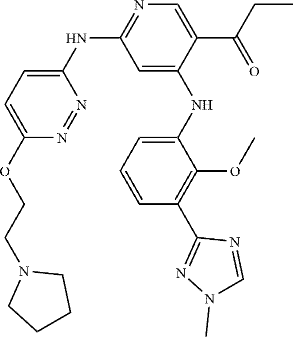 [SMILES: CCC(=O)c1cnc(Nc2ccc(OCCN3CCCC3)nn2)cc1Nc1cccc(-c2ncn(C)n2)c1OC] |
| 18DD | 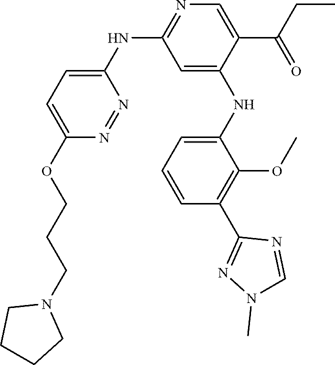 [SMILES: CCC(=O)c1cnc(Nc2ccc(OCCCN3CCCC3)nn2)cc1Nc1cccc(-c2ncn(C)n2)c1OC] |
| 18EE | 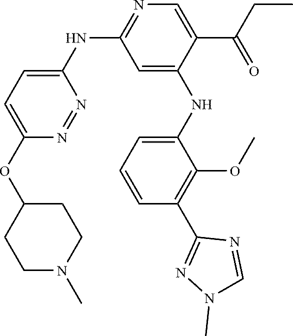 [SMILES: CCC(=O)c1cnc(Nc2ccc(OC3CCN(C)CC3)nn2)cc1Nc1cccc(-c2ncn(C)n2)c1OC] |
| 18FF | 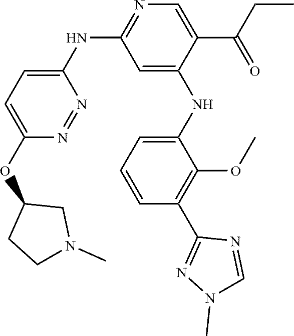 [SMILES: CCC(=O)c1cnc(Nc2ccc(O[C@@H]3CCN(C)C3)nn2)cc1Nc1cccc(-c2ncn(C)n2)c1OC] |
| 18GG | 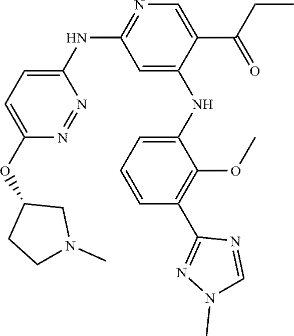 [SMILES: CCC(=O)c1cnc(Nc2ccc(O[C@H]3CCN(C)C3)nn2)cc1Nc1cccc(-c2ncn(C)n2)c1OC] |
| 18HH | 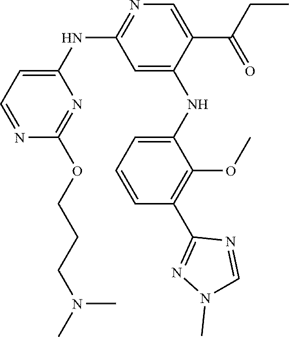 [SMILES: CCC(=O)c1cnc(Nc2ccnc(OCCCN(C)C)n2)cc1Nc1cccc(-c2ncn(C)n2)c1OC] |
| 18II | 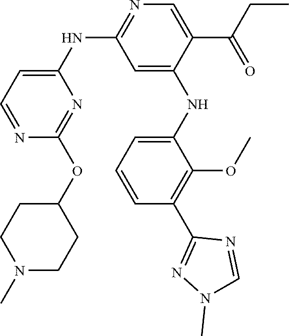 [SMILES: CCC(=O)c1cnc(Nc2ccnc(OC3CCN(C)CC3)n2)cc1Nc1cccc(-c2ncn(C)n2)c1OC] |
| 18JJ | 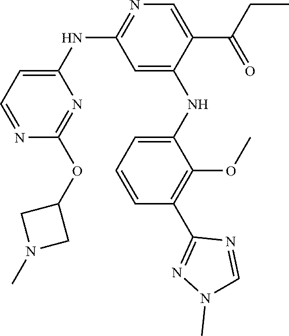 [SMILES: CCC(=O)c1cnc(Nc2ccnc(OC3CN(C)C3)n2)cc1Nc1cccc(-c2ncn(C)n2)c1OC] |
| 18KK | 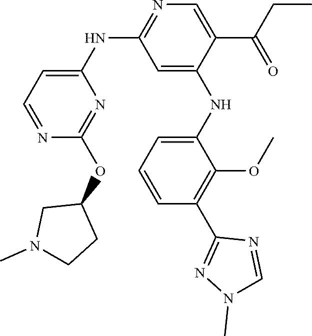 [SMILES: CCC(=O)c1cnc(Nc2ccnc(O[C@H]3CCN(C)C3)n2)cc1Nc1cccc(-c2ncn(C)n2)c1OC] |
| 18LL | 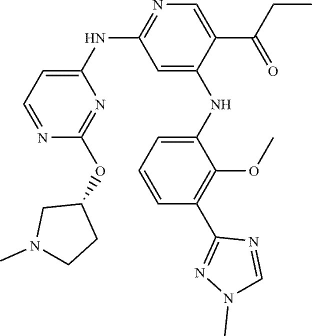 [SMILES: CCC(=O)c1cnc(Nc2ccnc(O[C@@H]3CCN(C)C3)n2)cc1Nc1cccc(-c2ncn(C)n2)c1OC] |
| 18MM | 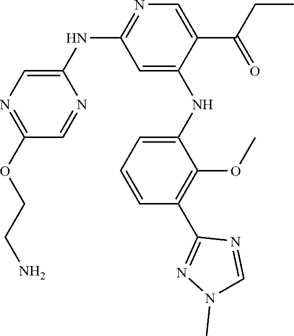 [SMILES: CCC(=O)c1cnc(Nc2cnc(OCCN)cn2)cc1Nc1cccc(-c2ncn(C)n2)c1OC] |
| 18NN | 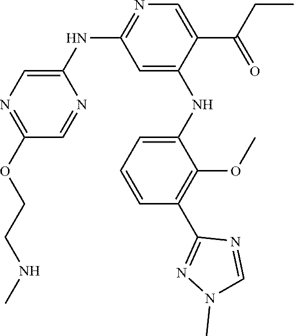 [SMILES: CCC(=O)c1cnc(Nc2cnc(OCCNC)cn2)cc1Nc1cccc(-c2ncn(C)n2)c1OC] |
| 1800 | 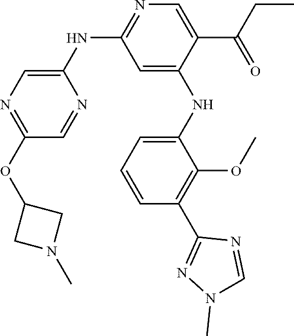 [SMILES: CCC(=O)c1cnc(Nc2cnc(OC3CN(C)C3)cn2)cc1Nc1cccc(-c2ncn(C)n2)c1OC] |
| 18PP | 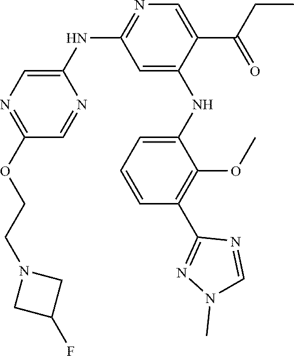 [SMILES: CCC(=O)c1cnc(Nc2cnc(OCCN3CC(F)C3)cn2)cc1Nc1cccc(-c2ncn(C)n2)c1OC] |
| 18QQ | 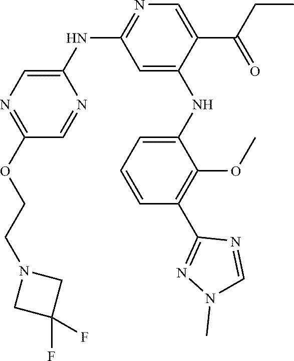 [SMILES: CCC(=O)c1cnc(Nc2cnc(OCCN3CC(F)(F)C3)cn2)cc1Nc1cccc(-c2ncn(C)n2)c1OC] |
| 18RR | 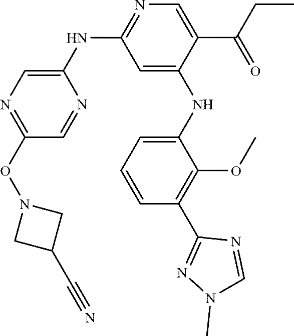 [SMILES: CCC(=O)c1cnc(Nc2cnc(ON3CC(C#N)C3)cn2)cc1Nc1cccc(-c2ncn(C)n2)c1OC] |
| 18SS | 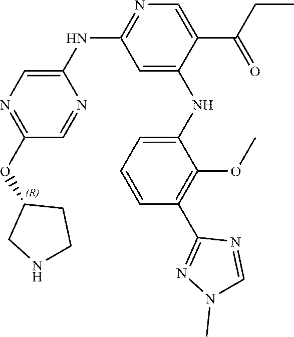 [SMILES: CCC(=O)c1cnc(Nc2cnc(O[C@@H]3CCNC3)cn2)cc1Nc1cccc(-c2ncn(C)n2)c1OC] |
| 18TT | 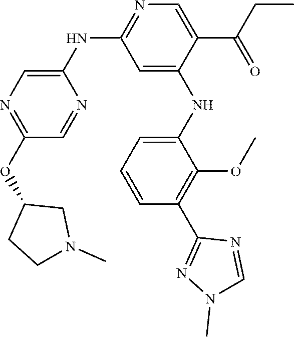 [SMILES: CCC(=O)c1cnc(Nc2cnc(O[C@H]3CCN(C)C3)cn2)cc1Nc1cccc(-c2ncn(C)n2)c1OC] |
| 18UU | 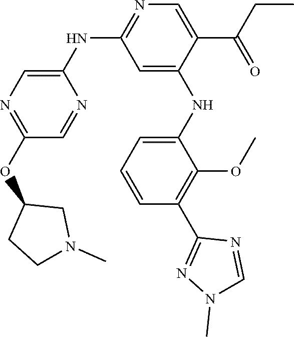 [SMILES: CCC(=O)c1cnc(Nc2cnc(O[C@@H]3CCN(C)C3)cn2)cc1Nc1cccc(-c2ncn(C)n2)c1OC] |
| 18VV | 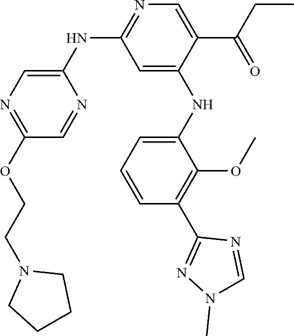 [SMILES: CCC(=O)c1cnc(Nc2cnc(OCCN3CCCC3)cn2)cc1Nc1cccc(-c2ncn(C)n2)c1OC] |
| 18WW | 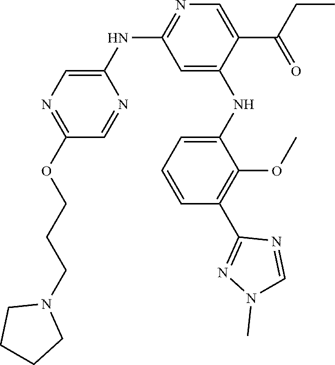 [SMILES: CCC(=O)c1cnc(Nc2cnc(OCCCN3CCCC3)cn2)cc1Nc1cccc(-c2ncn(C)n2)c1OC] |
| 18XX | 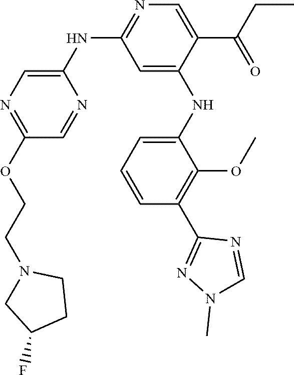 [SMILES: CCC(=O)c1cnc(Nc2cnc(OCCN3CC[C@H](F)C3)cn2)cc1Nc1cccc(-c2ncn(C)n2)c1OC] |
| 18YY | 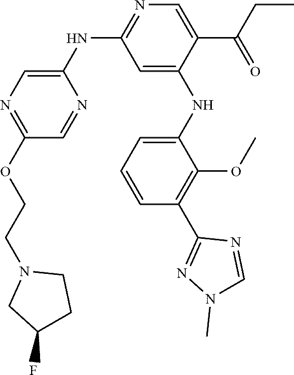 [SMILES: CCC(=O)c1cnc(Nc2cnc(OCCN3CC[C@@H](F)C3)cn2)cc1Nc1cccc(-c2ncn(C)n2)c1OC] |
| 18ZZ | 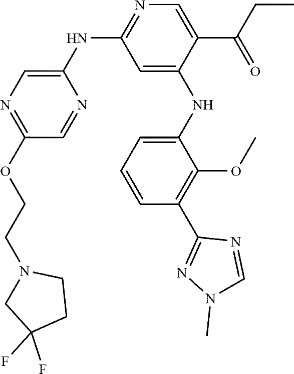 [SMILES: CCC(=O)c1cnc(Nc2cnc(OCCN3CCC(F)(F)C3)cn2)cc1Nc1cccc(-c2ncn(C)n2)c1OC] |
| 18 AAA | 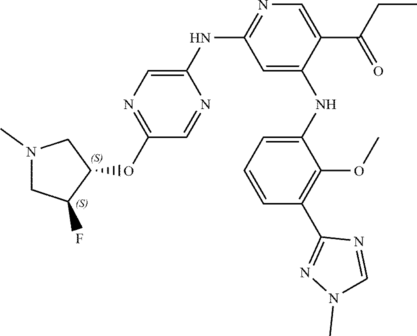 [SMILES: CCC(=O)c1cnc(Nc2cnc(O[C@H]3CN(C)C[C@@H]3F)cn2)cc1Nc1cccc(-c2ncn(C)n2)c1OC] |
| 18 BBB | 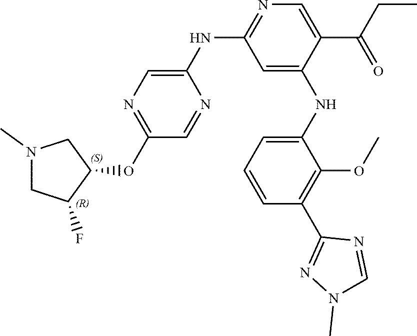 [SMILES: CCC(=O)c1cnc(Nc2cnc(O[C@H]3CN(C)C[C@H]3F)cn2)cc1Nc1cccc(-c2ncn(C)n2)c1OC] |
| 18 CCC | 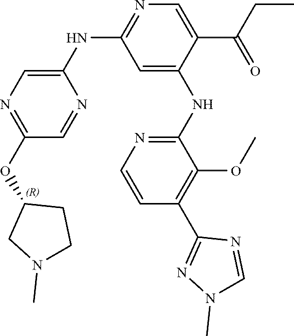 [SMILES: CCC(=O)c1cnc(Nc2cnc(O[C@@H]3CCN(C)C3)cn2)cc1Nc1nccc(-c2ncn(C)n2)c1OC] |
| 18 DDD | 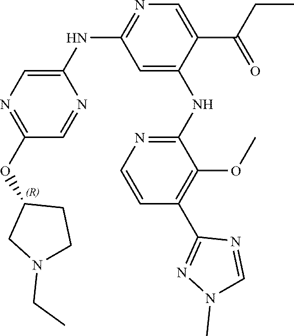 [SMILES: CCC(=O)c1cnc(Nc2cnc(O[C@@H]3CCN(CC)C3)cn2)cc1Nc1nccc(-c2ncn(C)n2)c1OC] |
| 18 EEE | 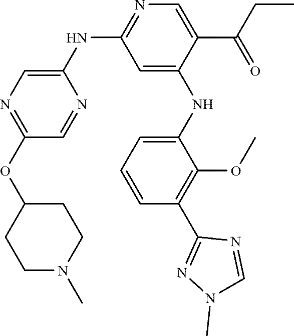 [SMILES: CCC(=O)c1cnc(Nc2cnc(OC3CCN(C)CC3)cn2)cc1Nc1cccc(-c2ncn(C)n2)c1OC] |
| 18 FFF | 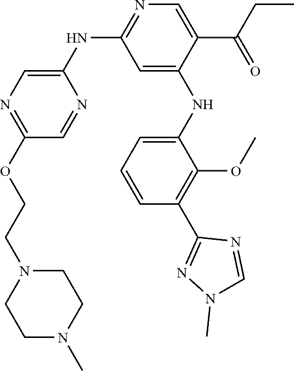 [SMILES: CCC(=O)c1cnc(Nc2cnc(OCCN3CCN(C)CC3)cn2)cc1Nc1cccc(-c2ncn(C)n2)c1OC] |
| 18 GGG | 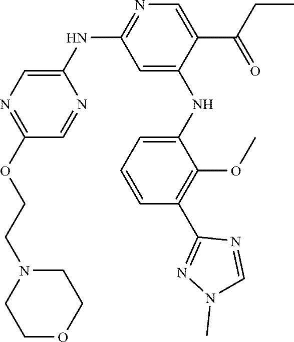 [SMILES: CCC(=O)c1cnc(Nc2cnc(OCCN3CCOCC3)cn2)cc1Nc1cccc(-c2ncn(C)n2)c1OC] |
| 18 HHH | 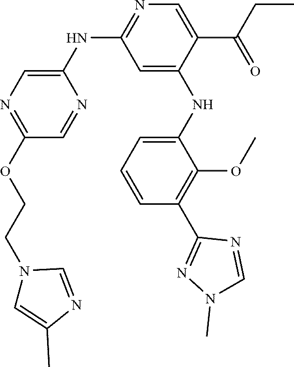 [SMILES: CCC(=O)c1cnc(Nc2cnc(OCCn3cnc(C)c3)cn2)cc1Nc1cccc(-c2ncn(C)n2)c1OC] |
| 18 III | 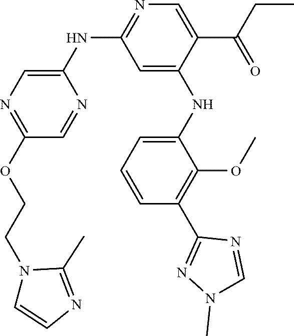 [SMILES: CCC(=O)c1cnc(Nc2cnc(OCCn3ccnc3C)cn2)cc1Nc1cccc(-c2ncn(C)n2)c1OC] |
| 18 JJJ | 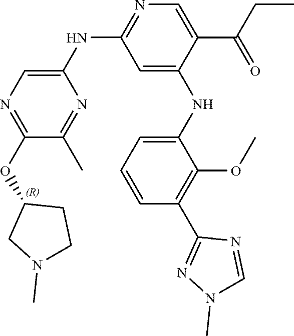 [SMILES: CCC(=O)c1cnc(Nc2cnc(O[C@@H]3CCN(C)C3)c(C)n2)cc1Nc1cccc(-c2ncn(C)n2)c1OC] |
| 18 KKK | 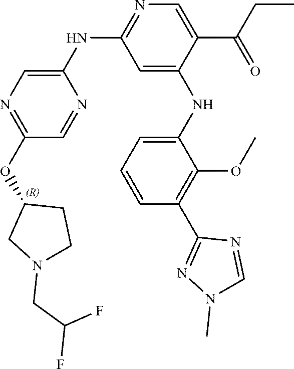 [SMILES: CCC(=O)c1cnc(Nc2cnc(O[C@@H]3CCN(CC(F)F)C3)cn2)cc1Nc1cccc(-c2ncn(C)n2)c1OC] |
| 18 LLL | 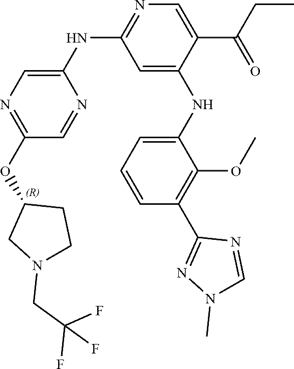 [SMILES: CCC(=O)c1cnc(Nc2cnc(O[C@@H]3CCN(CC(F)(F)F)C3)cn2)cc1Nc1cccc(-c2ncn(C)n2)c1OC] |
| 18 MMM | 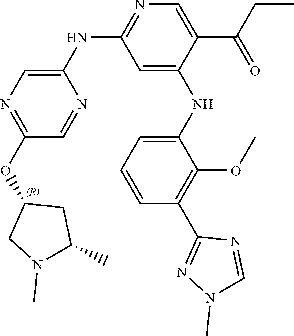 [SMILES: CCC(=O)c1cnc(Nc2cnc(O[C@@H]3C[C@H](C)N(C)C3)cn2)cc1Nc1cccc(-c2ncn(C)n2)c1OC] |
| 18NNN | 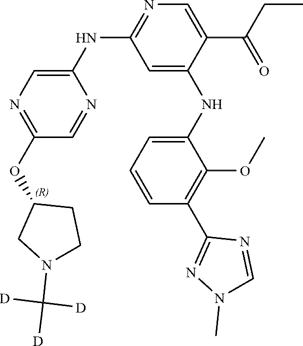 [SMILES: [2H]C([2H])([2H])N1CC[C@@H](Oc2cnc(Nc3cc(Nc4cccc(-c5ncn(C)n5)c4OC)c(C(=O)CC)cn3)cn2)C1] |
| 18 OOO | 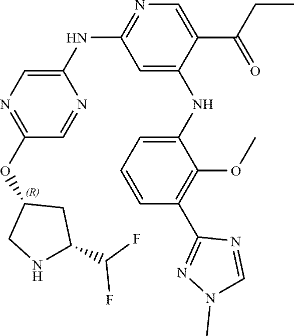 [SMILES: CCC(=O)c1cnc(Nc2cnc(O[C@H]3CN[C@@H](C(F)F)C3)cn2)cc1Nc1cccc(-c2ncn(C)n2)c1OC] |
| 18 PPP | 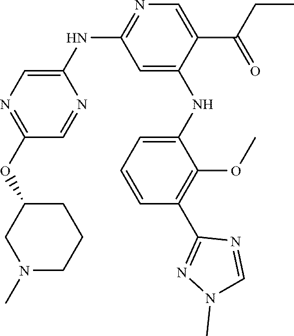 [SMILES: CCC(=O)c1cnc(Nc2cnc(O[C@@H]3CCCN(C)C3)cn2)cc1Nc1cccc(-c2ncn(C)n2)c1OC] |
| 18 QQQ | 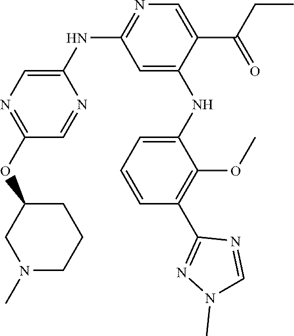 [SMILES: CCC(=O)c1cnc(Nc2cnc(O[C@H]3CCCN(C)C3)cn2)cc1Nc1cccc(-c2ncn(C)n2)c1OC] |
| 18 RRR | 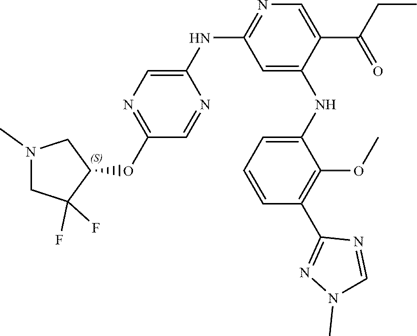 [SMILES: CCC(=O)c1cnc(Nc2cnc(O[C@H]3CN(C)CC3(F)F)cn2)cc1Nc1cccc(-c2ncn(C)n2)c1OC] |
| 18 SSS | 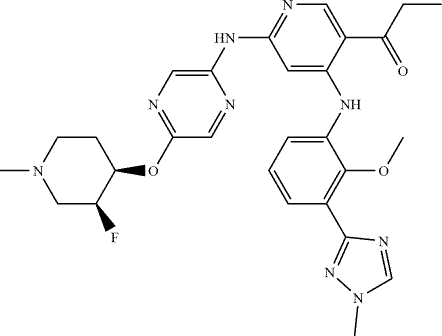 [SMILES: CCC(=O)c1cnc(Nc2cnc(O[C@@H]3CCN(C)C[C@@H]3F)cn2)cc1Nc1cccc(-c2ncn(C)n2)c1OC] |
| 18 TTT | 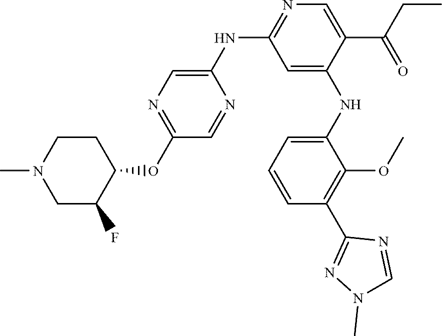 [SMILES: CCC(=O)c1cnc(Nc2cnc(O[C@H]3CCN(C)C[C@@H]3F)cn2)cc1Nc1cccc(-c2ncn(C)n2)c1OC] |
| 18 UUU | 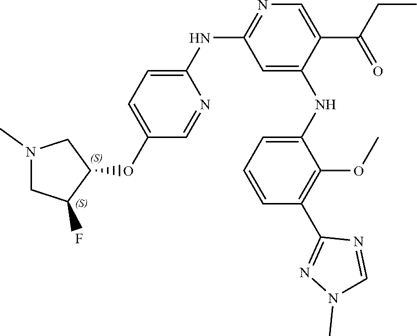 [SMILES: CCC(=O)c1cnc(Nc2ccc(O[C@H]3CN(C)C[C@@H]3F)cn2)cc1Nc1cccc(-c2ncn(C)n2)c1OC] |
| 18 VVV | 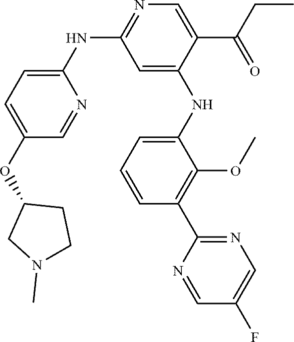 [SMILES: CCC(=O)c1cnc(Nc2ccc(O[C@@H]3CCN(C)C3)cn2)cc1Nc1cccc(-c2ncc(F)cn2)c1OC] |
| 18 WWW | 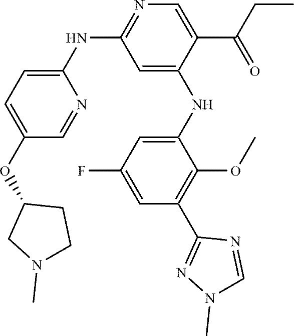 [SMILES: CCC(=O)c1cnc(Nc2ccc(O[C@@H]3CCN(C)C3)cn2)cc1Nc1cc(F)cc(-c2ncn(C)n2)c1OC] |
| 18 XXX | 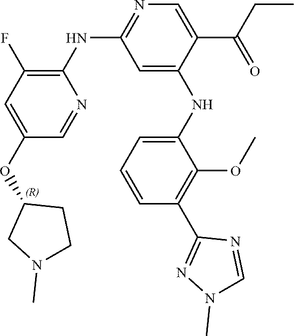 [SMILES: CCC(=O)c1cnc(Nc2ncc(O[C@@H]3CCN(C)C3)cc2F)cc1Nc1cccc(-c2ncn(C)n2)c1OC] |
| 18 YYY | 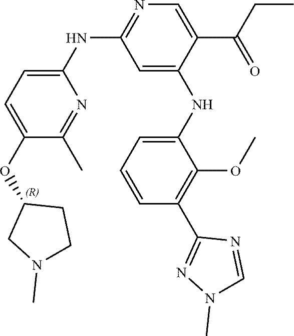 [SMILES: CCC(=O)c1cnc(Nc2ccc(O[C@@H]3CCN(C)C3)c(C)n2)cc1Nc1cccc(-c2ncn(C)n2)c1OC] |
| 18 ZZZ | 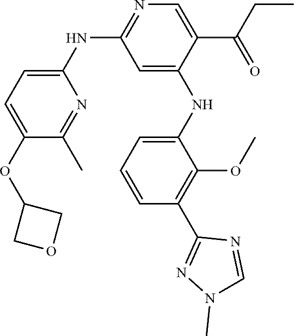 [SMILES: CCC(=O)c1cnc(Nc2ccc(OC3COC3)c(C)n2)cc1Nc1cccc(-c2ncn(C)n2)c1OC] |
| 18 AAAA | 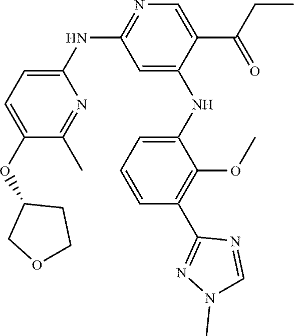 [SMILES: CCC(=O)c1cnc(Nc2ccc(O[C@@H]3CCOC3)c(C)n2)cc1Nc1cccc(-c2ncn(C)n2)c1OC] |
| 18 BBBB | 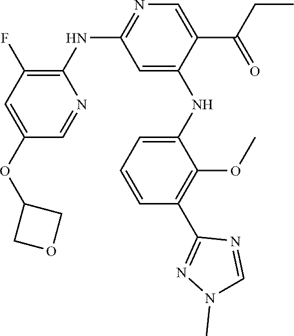 [SMILES: CCC(=O)c1cnc(Nc2ncc(OC3COC3)cc2F)cc1Nc1cccc(-c2ncn(C)n2)c1OC] |
| 18 CCCC | 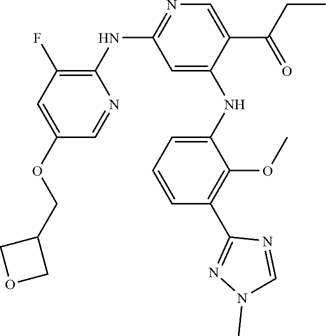 [SMILES: CCC(=O)c1cnc(Nc2ncc(OCC3COC3)cc2F)cc1Nc1cccc(-c2ncn(C)n2)c1OC] |
| 18 DDDD | 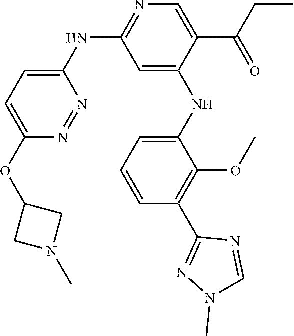 [SMILES: CCC(=O)c1cnc(Nc2ccc(OC3CN(C)C3)nn2)cc1Nc1cccc(-c2ncn(C)n2)c1OC] |
| 19A | 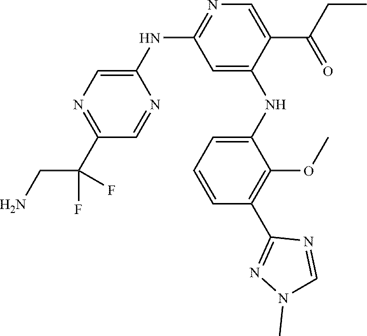 [SMILES: CCC(=O)c1cnc(Nc2cnc(C(F)(F)CN)cn2)cc1Nc1cccc(-c2ncn(C)n2)c1OC] |
| 19B | 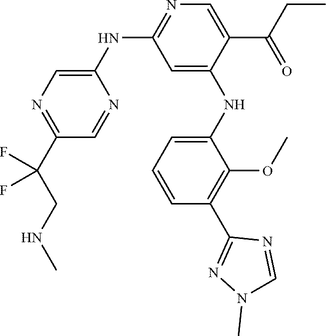 [SMILES: CCC(=O)c1cnc(Nc2cnc(C(F)(F)CNC)cn2)cc1Nc1cccc(-c2ncn(C)n2)c1OC] |
| 19C | 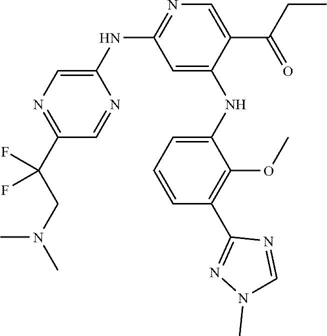 [SMILES: CCC(=O)c1cnc(Nc2cnc(C(F)(F)CN(C)C)cn2)cc1Nc1cccc(-c2ncn(C)n2)c1OC] |
| 19D | 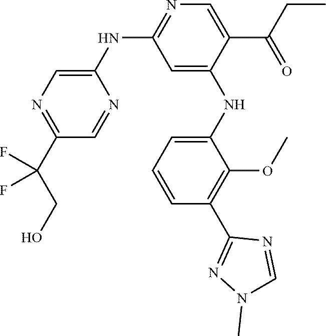 [SMILES: CCC(=O)c1cnc(Nc2cnc(C(F)(F)CO)cn2)cc1Nc1cccc(-c2ncn(C)n2)c1OC] |
| 19E | 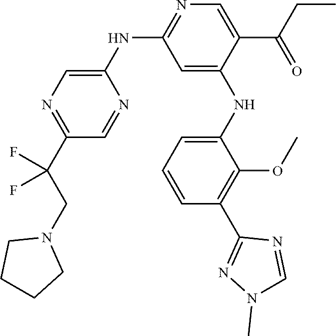 [SMILES: CCC(=O)c1cnc(Nc2cnc(C(F)(F)CN3CCCC3)cn2)cc1Nc1cccc(-c2ncn(C)n2)c1OC] |
| 20A | 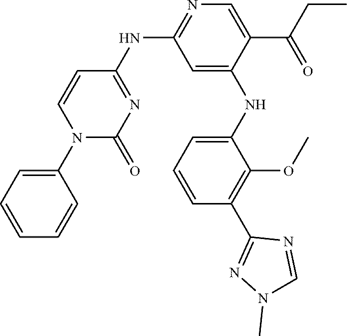 [SMILES: CCC(=O)c1cnc(Nc2ccn(-c3ccccc3)c(=O)n2)cc1Nc1cccc(-c2ncn(C)n2)c1OC] |
| 21A | 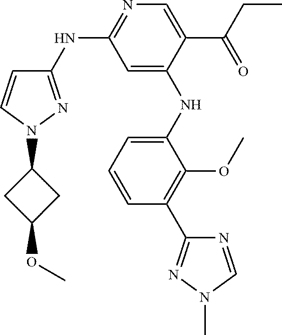 [SMILES: CCC(=O)c1cnc(Nc2ccn([C@H]3C[C@@H](OC)C3)n2)cc1Nc1cccc(-c2ncn(C)n2)c1OC] |
| 21B | 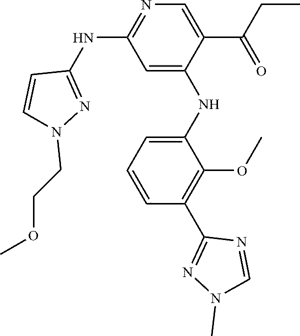 [SMILES: CCC(=O)c1cnc(Nc2ccn(CCOC)n2)cc1Nc1cccc(-c2ncn(C)n2)c1OC] |
| 21C | 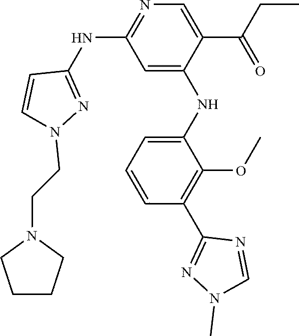 [SMILES: CCC(=O)c1cnc(Nc2ccn(CCN3CCCC3)n2)cc1Nc1cccc(-c2ncn(C)n2)c1OC] |
| 21D | 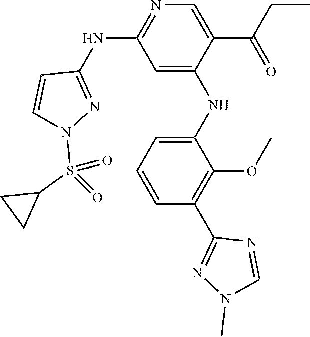 [SMILES: CCC(=O)c1cnc(Nc2ccn(S(=O)(=O)C3CC3)n2)cc1Nc1cccc(-c2ncn(C)n2)c1OC] |
| 21E | 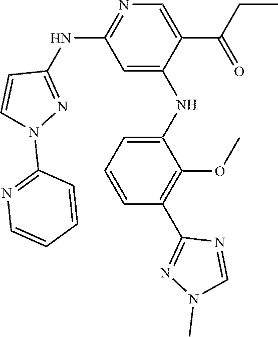 [SMILES: CCC(=O)c1cnc(Nc2ccn(-c3ccccn3)n2)cc1Nc1cccc(-c2ncn(C)n2)c1OC] |
| 21F | 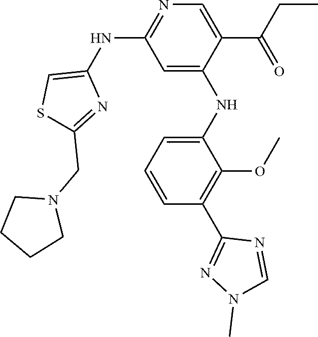 [SMILES: CCC(=O)c1cnc(Nc2csc(CN3CCCC3)n2)cc1Nc1cccc(-c2ncn(C)n2)c1OC] |
| 21G | 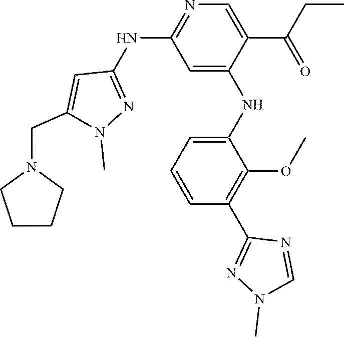 [SMILES: CCC(=O)c1cnc(Nc2cc(CN3CCCC3)n(C)n2)cc1Nc1cccc(-c2ncn(C)n2)c1OC] |
| 21H | 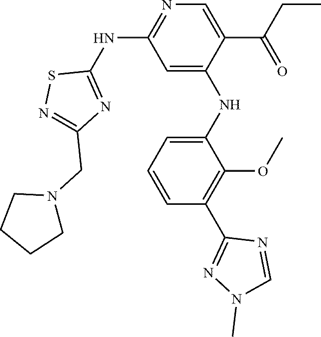 [SMILES: CCC(=O)c1cnc(Nc2nc(CN3CCCC3)ns2)cc1Nc1cccc(-c2ncn(C)n2)c1OC] |
| 211 | 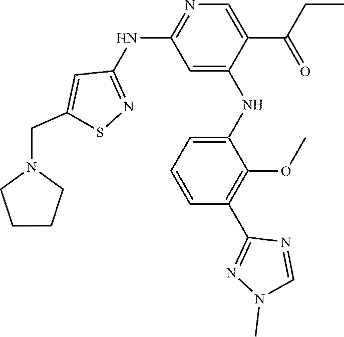 [SMILES: CCC(=O)c1cnc(Nc2cc(CN3CCCC3)sn2)cc1Nc1cccc(-c2ncn(C)n2)c1OC] |
| 21J | 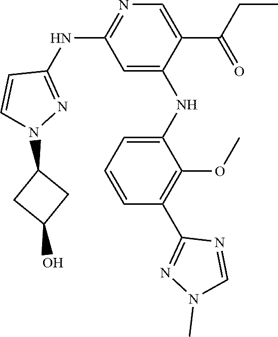 [SMILES: CCC(=O)c1cnc(Nc2ccn([C@H]3C[C@@H](O)C3)n2)cc1Nc1cccc(-c2ncn(C)n2)c1OC] |
| 21K | 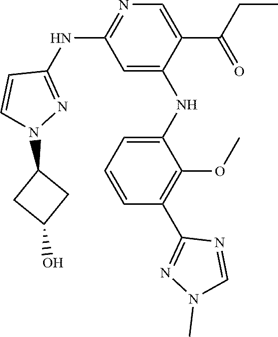 [SMILES: CCC(=O)c1cnc(Nc2ccn([C@H]3C[C@H](O)C3)n2)cc1Nc1cccc(-c2ncn(C)n2)c1OC] |
| 21L | 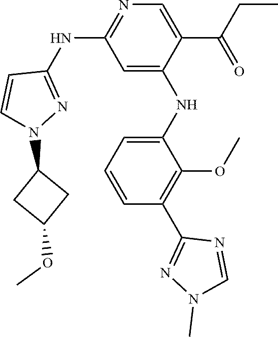 [SMILES: CCC(=O)c1cnc(Nc2ccn([C@H]3C[C@H](OC)C3)n2)cc1Nc1cccc(-c2ncn(C)n2)c1OC] |
| 21M | 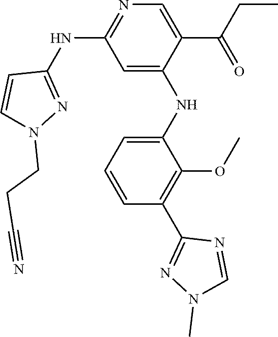 [SMILES: CCC(=O)c1cnc(Nc2ccn(CCC#N)n2)cc1Nc1cccc(-c2ncn(C)n2)c1OC] |
| 21N | 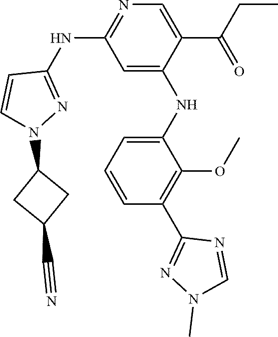 [SMILES: CCC(=O)c1cnc(Nc2ccn([C@H]3C[C@@H](C#N)C3)n2)cc1Nc1cccc(-c2ncn(C)n2)c1OC] |
| 210 | 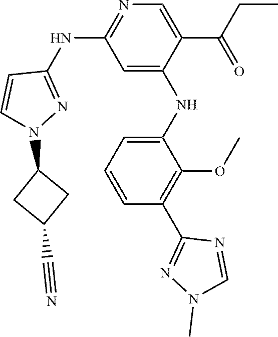 [SMILES: CCC(=O)c1cnc(Nc2ccn([C@H]3C[C@H](C#N)C3)n2)cc1Nc1cccc(-c2ncn(C)n2)c1OC] |
| 21P | 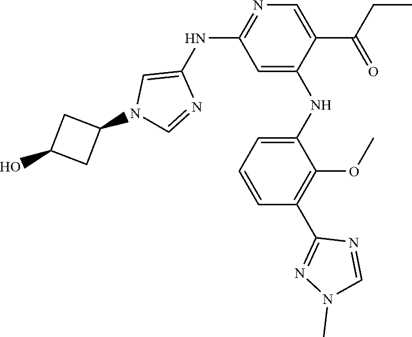 [SMILES: CCC(=O)c1cnc(Nc2cn([C@H]3C[C@@H](O)C3)cn2)cc1Nc1cccc(-c2ncn(C)n2)c1OC] |
| 21Q | 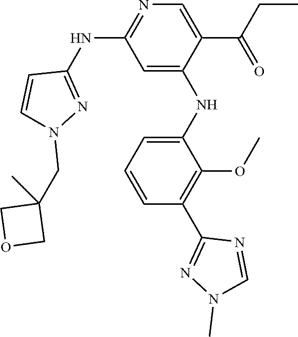 [SMILES: CCC(=O)c1cnc(Nc2ccn(CC3(C)COC3)n2)cc1Nc1cccc(-c2ncn(C)n2)c1OC] |
| 21R | 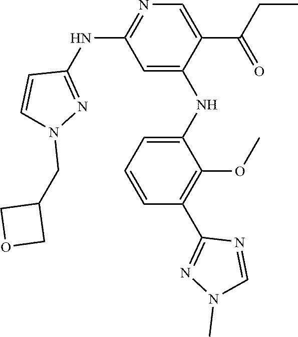 [SMILES: CCC(=O)c1cnc(Nc2ccn(CC3COC3)n2)cc1Nc1cccc(-c2ncn(C)n2)c1OC] |
| 21S | 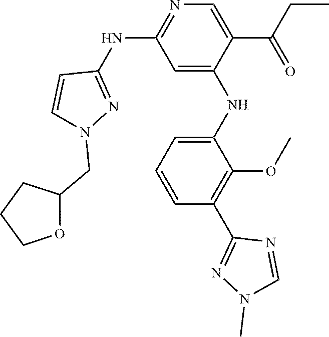 [SMILES: CCC(=O)c1cnc(Nc2ccn(CC3CCCO3)n2)cc1Nc1cccc(-c2ncn(C)n2)c1OC] |
| 21T | 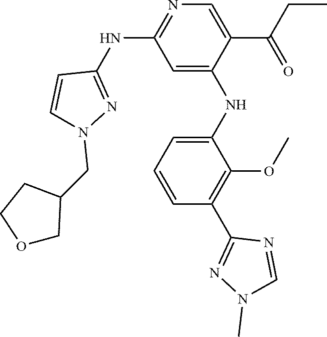 [SMILES: CCC(=O)c1cnc(Nc2ccn(CC3CCOC3)n2)cc1Nc1cccc(-c2ncn(C)n2)c1OC] |
| 21U | 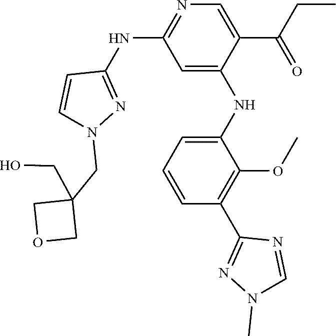 [SMILES: CCC(=O)c1cnc(Nc2ccn(CC3(CO)COC3)n2)cc1Nc1cccc(-c2ncn(C)n2)c1OC] |
| 21V | 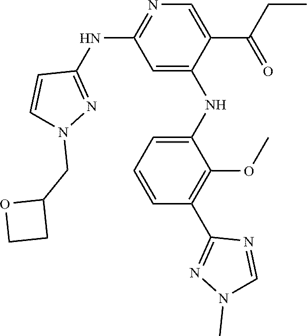 [SMILES: CCC(=O)c1cnc(Nc2ccn(CC3CCO3)n2)cc1Nc1cccc(-c2ncn(C)n2)c1OC] |
| 21W | 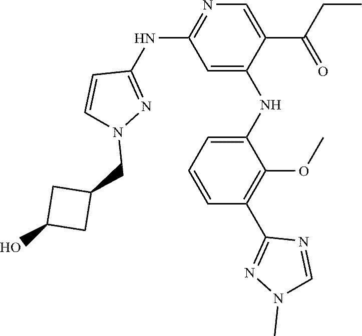 [SMILES: CCC(=O)c1cnc(Nc2ccn(C[C@H]3C[C@@H](O)C3)n2)cc1Nc1cccc(-c2ncn(C)n2)c1OC] |
| 21X | 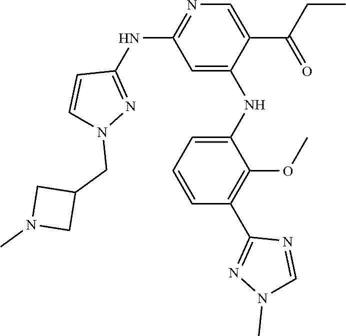 [SMILES: CCC(=O)c1cnc(Nc2ccn(CC3CN(C)C3)n2)cc1Nc1cccc(-c2ncn(C)n2)c1OC] |
| 21Y | 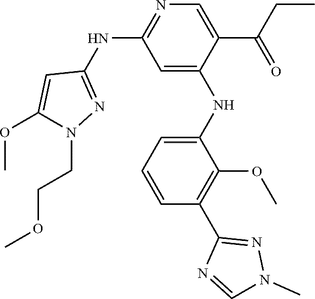 [SMILES: CCC(=O)c1cnc(Nc2cc(OC)n(CCOC)n2)cc1Nc1cccc(-c2ncn(C)n2)c1OC] |
| 21Z | 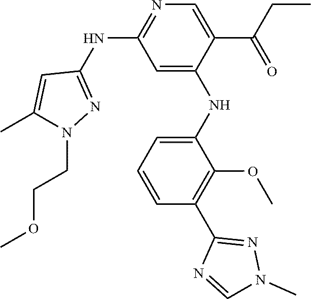 [SMILES: CCC(=O)c1cnc(Nc2cc(C)n(CCOC)n2)cc1Nc1cccc(-c2ncn(C)n2)c1OC] |
| 21 AA | 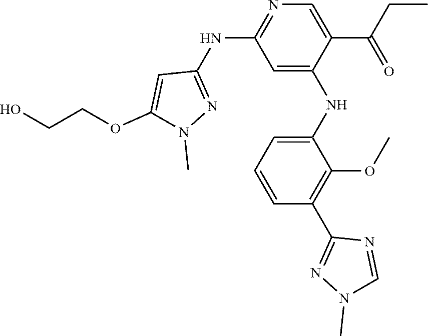 [SMILES: CCC(=O)c1cnc(Nc2cc(OCCO)n(C)n2)cc1Nc1cccc(-c2ncn(C)n2)c1OC] |
| 21 BB | 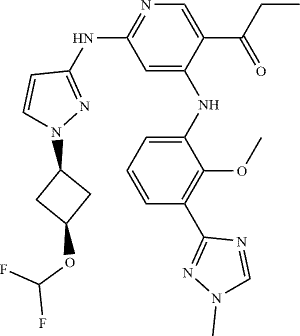 [SMILES: CCC(=O)c1cnc(Nc2ccn([C@H]3C[C@@H](OC(F)F)C3)n2)cc1Nc1cccc(-c2ncn(C)n2)c1OC] |
| 21 CC | 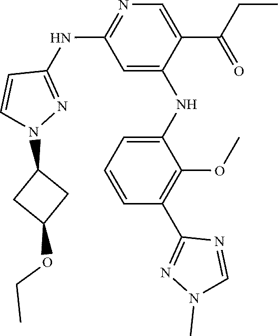 [SMILES: CCO[C@H]1C[C@@H](n2ccc(Nc3cc(Nc4cccc(-c5ncn(C)n5)c4OC)c(C(=O)CC)cn3)n2)C1] |
| 21 DD | 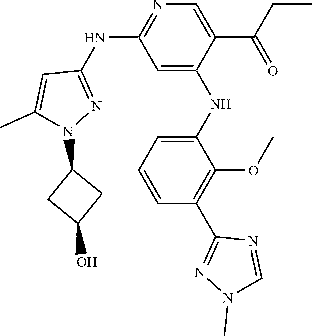 [SMILES: CCC(=O)c1cnc(Nc2cc(C)n([C@H]3C[C@@H](O)C3)n2)cc1Nc1cccc(-c2ncn(C)n2)c1OC] |
| 21 EE | 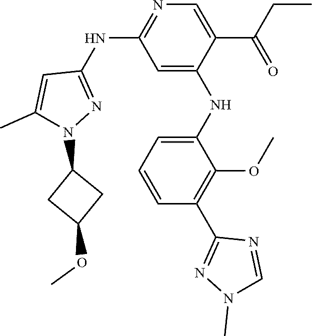 [SMILES: CCC(=O)c1cnc(Nc2cc(C)n([C@H]3C[C@@H](OC)C3)n2)cc1Nc1cccc(-c2ncn(C)n2)c1OC] |
| 21 FF | 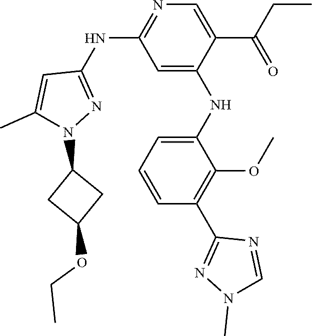 [SMILES: CCO[C@H]1C[C@@H](n2nc(Nc3cc(Nc4cccc(-c5ncn(C)n5)c4OC)c(C(=O)CC)cn3)cc2C)C1] |
| 21 GG | 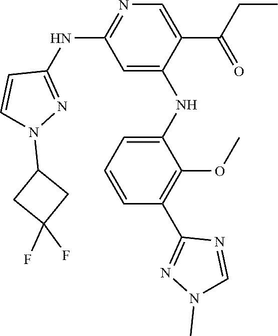 [SMILES: CCC(=O)c1cnc(Nc2ccn(C3CC(F)(F)C3)n2)cc1Nc1cccc(-c2ncn(C)n2)c1OC] |
| 22A | 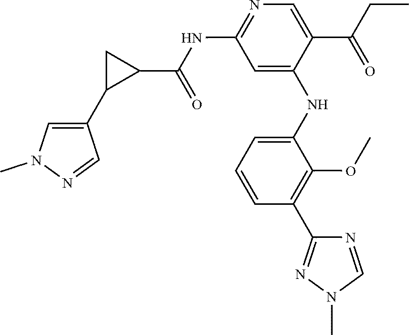 [SMILES: CCC(=O)c1cnc(NC(=O)C2CC2c2cnn(C)c2)cc1Nc1cccc(-c2ncn(C)n2)c1OC] |
| 22B | 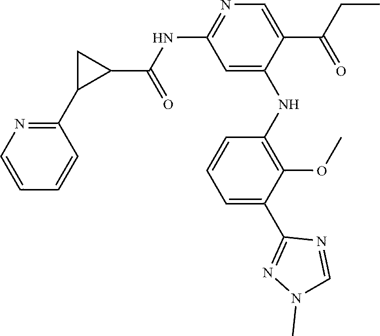 [SMILES: CCC(=O)c1cnc(NC(=O)C2CC2c2ccccn2)cc1Nc1cccc(-c2ncn(C)n2)c1OC] |
| 22C | 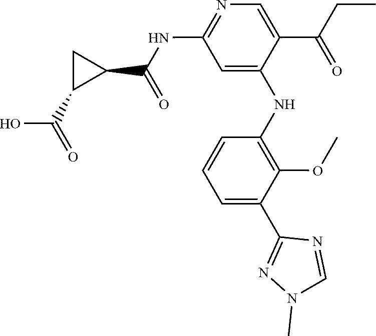 [SMILES: CCC(=O)c1cnc(NC(=O)[C@@H]2C[C@H]2C(=O)O)cc1Nc1cccc(-c2ncn(C)n2)c1OC] |
| 22D | 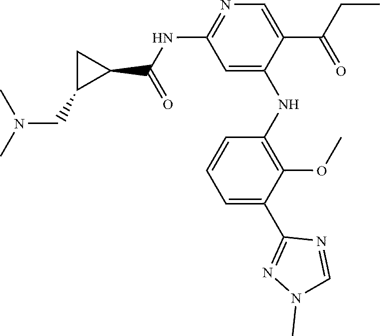 [SMILES: CCC(=O)c1cnc(NC(=O)[C@@H]2C[C@H]2CN(C)C)cc1Nc1cccc(-c2ncn(C)n2)c1OC] |
| 22E | 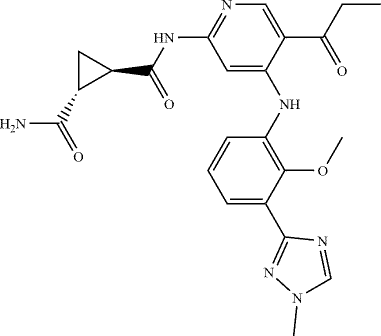 [SMILES: CCC(=O)c1cnc(NC(=O)[C@@H]2C[C@H]2C(N)=O)cc1Nc1cccc(-c2ncn(C)n2)c1OC] |
| 22F | 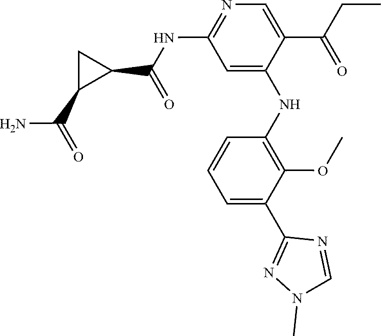 [SMILES: CCC(=O)c1cnc(NC(=O)[C@@H]2C[C@@H]2C(N)=O)cc1Nc1cccc(-c2ncn(C)n2)c1OC] |
| 22G | 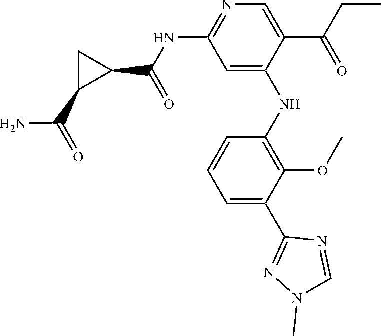 [SMILES: CCC(=O)c1cnc(NC(=O)[C@@H]2C[C@@H]2C(N)=O)cc1Nc1cccc(-c2ncn(C)n2)c1OC] |
| 22H | 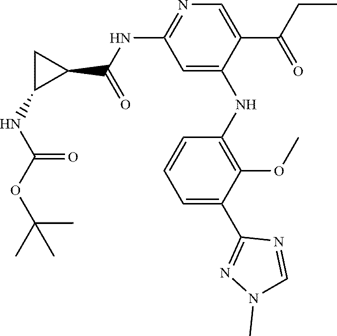 [SMILES: CCC(=O)c1cnc(NC(=O)[C@@H]2C[C@H]2NC(=O)OC(C)(C)C)cc1Nc1cccc(-c2ncn(C)n2)c1OC] |
| 22I | 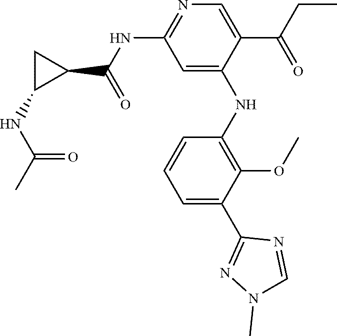 [SMILES: CCC(=O)c1cnc(NC(=O)[C@@H]2C[C@H]2NC(C)=O)cc1Nc1cccc(-c2ncn(C)n2)c1OC] |
| 22J | 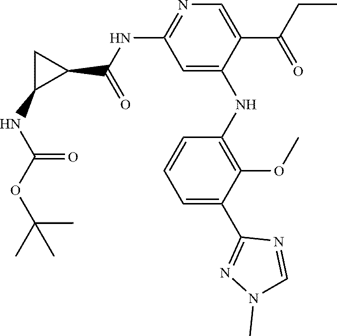 [SMILES: CCC(=O)c1cnc(NC(=O)[C@@H]2C[C@@H]2NC(=O)OC(C)(C)C)cc1Nc1cccc(-c2ncn(C)n2)c1OC] |
| 22K | 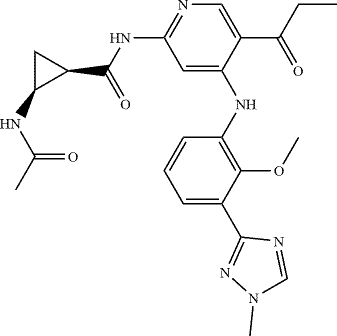 [SMILES: CCC(=O)c1cnc(NC(=O)[C@@H]2C[C@@H]2NC(C)=O)cc1Nc1cccc(-c2ncn(C)n2)c1OC] |
| 22L | 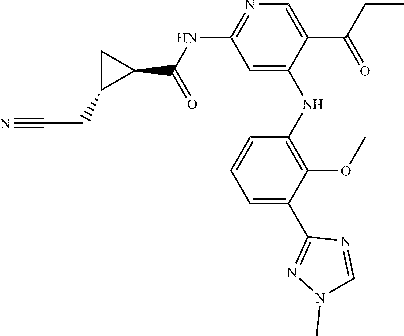 [SMILES: CCC(=O)c1cnc(NC(=O)[C@@H]2C[C@H]2CC#N)cc1Nc1cccc(-c2ncn(C)n2)c1OC] |
| 22M | 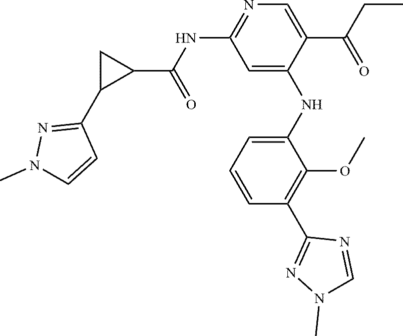 [SMILES: CCC(=O)c1cnc(NC(=O)C2CC2c2ccn(C)n2)cc1Nc1cccc(-c2ncn(C)n2)c1OC] |
| 22N | 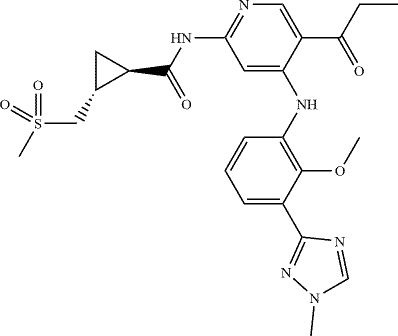 [SMILES: CCC(=O)c1cnc(NC(=O)[C@@H]2C[C@H]2CS(C)(=O)=O)cc1Nc1cccc(-c2ncn(C)n2)c1OC] |
| 23A | 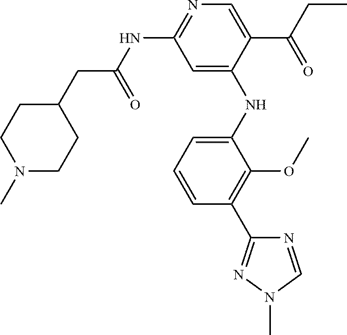 [SMILES: CCC(=O)c1cnc(NC(=O)CC2CCN(C)CC2)cc1Nc1cccc(-c2ncn(C)n2)c1OC] |
| 23B | 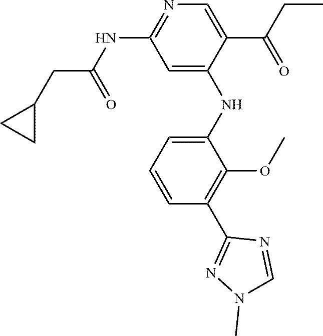 [SMILES: CCC(=O)c1cnc(NC(=O)CC2CC2)cc1Nc1cccc(-c2ncn(C)n2)c1OC] |
| 23C | 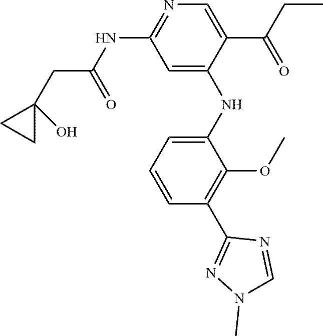 [SMILES: CCC(=O)c1cnc(NC(=O)CC2(O)CC2)cc1Nc1cccc(-c2ncn(C)n2)c1OC] |
| 23D | 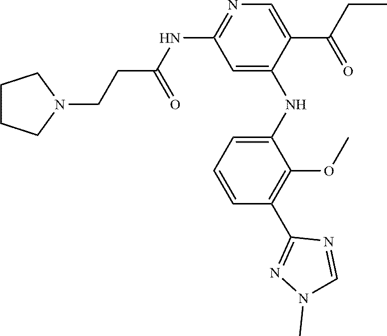 [SMILES: CCC(=O)c1cnc(NC(=O)CCN2CCCC2)cc1Nc1cccc(-c2ncn(C)n2)c1OC] |
| 23E | 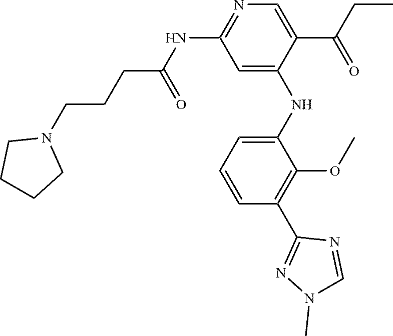 [SMILES: CCC(=O)c1cnc(NC(=O)CCCN2CCCC2)cc1Nc1cccc(-c2ncn(C)n2)c1OC] |
| 23F | 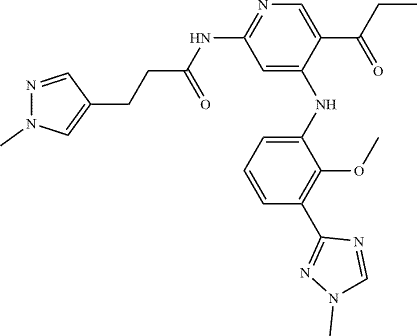 [SMILES: CCC(=O)c1cnc(NC(=O)CCc2cnn(C)c2)cc1Nc1cccc(-c2ncn(C)n2)c1OC] |
| 23G | 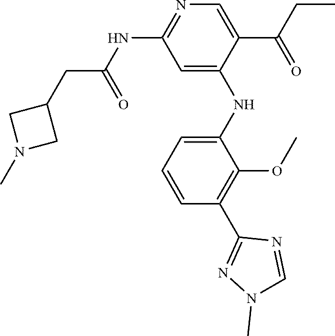 [SMILES: CCC(=O)c1cnc(NC(=O)CC2CN(C)C2)cc1Nc1cccc(-c2ncn(C)n2)c1OC] |
| 23H | 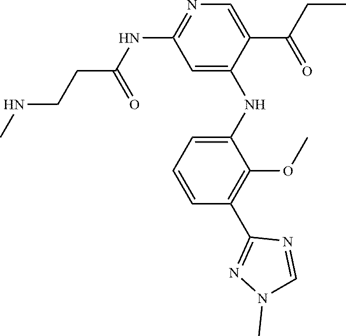 [SMILES: CCC(=O)c1cnc(NC(=O)CCNC)cc1Nc1cccc(-c2ncn(C)n2)c1OC] |
| 24A | 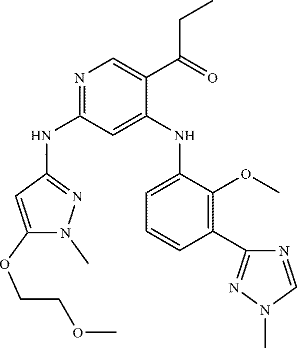 [SMILES: CCC(=O)c1cnc(Nc2cc(OCCOC)n(C)n2)cc1Nc1cccc(-c2ncn(C)n2)c1OC] |
| 25A | 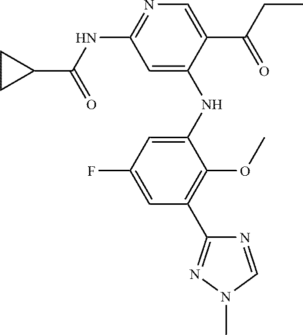 [SMILES: CCC(=O)c1cnc(NC(=O)C2CC2)cc1Nc1cc(F)cc(-c2ncn(C)n2)c1OC] |
| 25B | 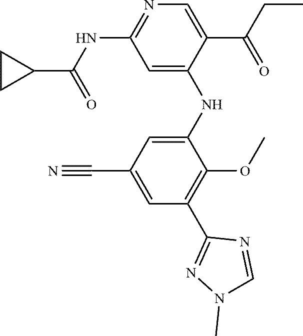 [SMILES: CCC(=O)c1cnc(NC(=O)C2CC2)cc1Nc1cc(C#N)cc(-c2ncn(C)n2)c1OC] |
| 25C | 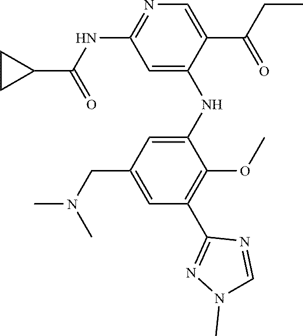 [SMILES: CCC(=O)c1cnc(NC(=O)C2CC2)cc1Nc1cc(CN(C)C)cc(-c2ncn(C)n2)c1OC] |
| 25D | 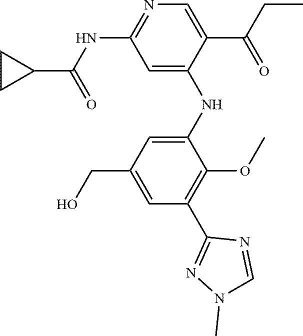 [SMILES: CCC(=O)c1cnc(NC(=O)C2CC2)cc1Nc1cc(CO)cc(-c2ncn(C)n2)c1OC] |
| 25E | 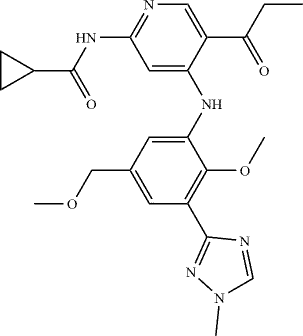 [SMILES: CCC(=O)c1cnc(NC(=O)C2CC2)cc1Nc1cc(COC)cc(-c2ncn(C)n2)c1OC] |
| 25F | 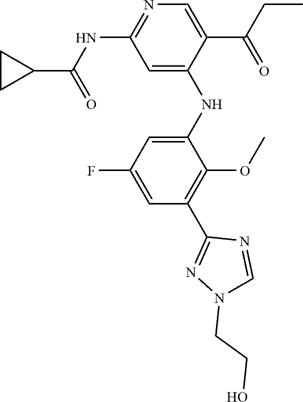 [SMILES: CCC(=O)c1cnc(NC(=O)C2CC2)cc1Nc1cc(F)cc(-c2ncn(CCO)n2)c1OC] |
| 26A | 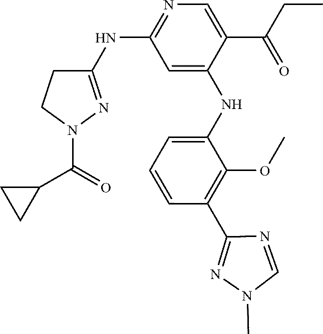 [SMILES: CCC(=O)c1cnc(NC2=NN(C(=O)C3CC3)CC2)cc1Nc1cccc(-c2ncn(C)n2)c1OC] |

In some embodiments are compounds having the structure of any one of Formulas (I), (II), (III), (III-i), (III-ii), (IV), (IV-i), (IV-ii), (V), (VI), (VI-A), (VI-A-i), (VI-A-ii), (VI-B), (VI-B-i), or (VI-B-ii) or of Table 1. In some embodiments are pharmaceutically acceptable salts of compounds having the structure of any one of Formulas (I), (II), (III), (III-i), (III-ii), (IV), (IV-i), (IV-ii), (V), (VI), (VI-A), (VI-A-i), (VI-A-ii), (VI-B), (VI-B-i), or (VI-B-ii) or of Table 1. In some embodiments are hydrates of compounds having the structure of any one of Formulas (I), (II), (III), (III-i), (III-ii), (IV), (IV-i), (IV-ii), (V), (VI), (VI-A), (VI-A-i), (VI-A-ii), (VI-B), (VI-B-i), or (VI-B-ii) or of Table 1. In some embodiments are isomers of compounds having the structure of any one of Formulas (I), (II), (III), (III-i), (III-ii), (IV), (IV-i), (IV-ii), (V), (VI), (VI-A), (VI-A-i), (VI-A-ii), (VI-B), (VI-B-i), or (VI-B-ii) or of Table 1. In some embodiments are atropisomers of compounds having the structure of any one of Formulas (I), (II), (III), (III-i), (III-ii), (IV), (IV-i), (IV-ii), (V), (VI), (VI-A), (VI-A-i), (VI-A-ii), (VI-B), (VI-B-i), or (VI-B-ii) or of Table 1. In some embodiments are tautomers of compounds having the structure of any one of Formulas (I), (II), (III), (III-i), (III-ii), (IV), (IV-i), (IV-ii), (V), (VI), (VI-A), (VI-A-i), (VI-A-ii), (VI-B), (VI-B-i), or (VI-B-ii) or of Table 1. In some embodiments are racemates of compounds having the structure of any one of Formulas (I), (II), (III), (III-i), (III-ii), (IV), (IV-i), (IV-ii), (V), (VI), (VI-A), (VI-A-i), (VI-A-ii), (VI-B), (VI-B-i), or (VI-B-ii) or of Table 1. In some embodiments are isotopic forms of compounds having the structure of any one of Formulas (I), (II), (III), (III-i), (111-ii), (IV), (IV-i), (IV-ii), (V), (VI), (VI-A), (VI-A-i), (VI-A-ii), (VI-B), (VI-B-i), or (VI-B-ii) or of Table 1.

In further embodiments, pharmaceutical compositions are provided comprising a compound having the structure of any one of Formulas (I), (II), (III), (III-i), (III-ii), (IV), (IV-i), (IV-ii), (V), (VI), (VI-A), (VI-A-i), (VI-A-ii), (VI-B), (VI-B-i), or (VI-B-ii) or of Table 1, or a pharmaceutically acceptable salt, solvate, hydrate, isomer, tautomer, racemate or isotope, and at least one pharmaceutically acceptable excipient.

In some embodiments are methods of treating diseases, comprising administering to a patient in need thereof, a therapeutically effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or an isomer, a stereoisomer, tautomer, solvate or prodrug thereof, or a pharmaceutically acceptable salt thereof.

In some embodiments are methods of treating diseases, comprising administering to a patient suffering from the disease, a therapeutically effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or an isomer, a stereoisomer, tautomer, solvate or prodrug thereof, or a pharmaceutically acceptable salt thereof. In some embodiments are methods of treating a disease responsive to the inhibition of TYK2 kinase activity, comprising administering to a patient suffering from the disease, a therapeutically effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or an isomer, a stereoisomer, tautomer, solvate or prodrug thereof, or a pharmaceutically acceptable salt thereof. In some embodiments the disease is an inflammatory disease. In some embodiments the disease is asthma, inflammatory bowel disease, Crohn’s disease, ulcerative colitis, rheumatoid arthritis, psoriasis, allergic rhinitis, atopic dermatitis, contact dermatitis, delayed hypersensitivity reactions, lupus or multiple sclerosis.

In some embodiments are methods of treating diseases, comprising administering to a patient suffering from the disease, a therapeutically effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or an isomer, a stereoisomer, tautomer, solvate or prodrug thereof, or a pharmaceutically acceptable salt thereof, and a second therapeutic agent.

In some embodiments are kits, comprising a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or an isomer, a stereoisomer, tautomer, solvate or prodrug thereof, or a pharmaceutically acceptable salt thereof, and instructions for use.

In some embodiments are compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or an isomer, a stereoisomer, tautomer, solvate or prodrug thereof, or a pharmaceutically acceptable salt thereof, for use in treating an inflammatory disease, in particular wherein the inflammatory disease is asthma, inflammatory bowel disease, Crohn’s disease, ulcerative colitis, rheumatoid arthritis, psoriasis, allergic rhinitis, atopic dermatitis, contact dermatitis, delayed hypersensitivity reactions, lupus or multiple sclerosis.

In some embodiments are uses of compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or an isomer, a stereoisomer, tautomer, solvate or prodrug thereof, or a pharmaceutically acceptable salt thereof, in the manufacture of a medicament for the treatment of an inflammatory disease, in particular wherein the inflammatory disease is asthma, inflammatory bowel disease, Crohn’s disease, ulcerative colitis, rheumatoid arthritis, psoriasis, allergic rhinitis, atopic dermatitis, contact dermatitis, delayed hypersensitivity reactions, lupus or multiple sclerosis.

Also described herein are pharmaceutical compositions comprising compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or pharmaceutically acceptable salts, solvates, hydrates, isomers, tautomers, racemates, or isotopes thereof, and at least one pharmaceutically acceptable excipient. In some embodiments, the pharmaceutical compositions comprise compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or pharmaceutically acceptable salts, solvates, hydrates, isomers, tautomers, racemates, or isotopes thereof, and at least one pharmaceutically acceptable excipient.

Also described herein are uses of compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or a pharmaceutically acceptable salts, solvates, hydrates, isomers, tautomers, racemates or isotopes thereof, in the manufacture of a medicament. In some embodiments the medicament is for the treatment of asthma. In some embodiments the medicament is for the treatment of inflammatory bowel disease. In some embodiments the medicament is for the treatment of Crohn’s disease. In some embodiments the medicament is for the treatment of ulcerative colitis. In some embodiments the medicament is for the treatment of rheumatoid arthritis. In some embodiments the medicament is for the treatment of psoriasis. In some embodiments the medicament is for the treatment of allergic rhinitis. In some embodiments the medicament is for the treatment of atopic dermatitis. In some embodiments the medicament is for the treatment of contact dermatitis. In some embodiments the medicament is for the treatment of delayed hypersensitivity reactions. In some embodiments the medicament is for the treatment of lupus. In some embodiments the medicament is for the treatment of multiple sclerosis.

“Isomer” as used herein to encompasses all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the disclosure. The isomers resulting from the presence of a chiral center comprise a pair of non-superimposable isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same Formula. For example, the isolated isomer may be at least about 80%, at least 80% or at least 85% pure. In other embodiments, the isolated isomer is at least 90% pure or at least 98% pure, or at least 99% pure by weight.

“Substantially enantiomerically or diastereomerically” pure means a level of enantiomeric or diastereomeric enrichment of one enantiomer with respect to the other enantiomer or diastereomer of at least about 80%, and more specifically in excess of 80%, 85%, 90%, 95%, 98%, 99%, 99.5% or 99.9%.

As used herein the terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out).

Unless stated to the contrary, a Formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine 19 is longest-lived. Thus, an isotope of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 includes, but is not limited to, compounds having the structure of Formula wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the disclosure include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, trifluoroacetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, (βhydroxybutyric, salicylic, -galactaric, and galacturonic acid.

Although pharmaceutically unacceptable salts are not generally useful as medicaments, such salts may be useful, for example as intermediates in the synthesis of compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, for example in their purification by recrystallization.

In certain embodiments, the disclosure provides a pharmaceutical composition comprising a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or a pharmaceutically acceptable isomer, racemate, hydrate, solvate, isotope, or salt thereof, together with at least one pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, homolog or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

In another embodiment, there are provided methods of making a composition of a compound described herein including formulating a compound of the disclosure with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation.

As used herein, the term “pharmaceutically acceptable carrier” refers to any ingredient other than the disclosed compounds, or a pharmaceutically acceptable isomer, racemate, hydrate, solvate, homolog or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the disclosure to the appropriate or desired site of action, such as oral, nasal, pulmonary, buccal, subdermal, intradermal, transdermal, or parenteral, e.g., rectal, depot, subcutaneous, intravenous, inhalation of a dry powder form or a nebulized form, intraurethral, intramuscular, intranasal, ophthalmic solution, or an ointment, the oral route being preferred.

Dosage forms can be administered once a day, or more than once a day, such as twice or thrice daily. Alternatively, dosage forms can be administered less frequently than daily, such as every other day, or weekly, if found to be advisable by a prescribing physician. Dosing regimens include, for example, dose titration to the extent necessary or useful for the indication to be treated, thus allowing the patient’s body to adapt to the treatment and/or to minimize or avoid unwanted side effects associated with the treatment. Other dosage forms include delayed or controlled-release forms. Suitable dosage regimens and/or forms include those set out, for example, in the latest edition of the Physicians’ Desk Reference, incorporated herein by reference.

As used herein, the term “administering” or “administration” refers to providing a compound, a pharmaceutical composition comprising the same, to a subject by any acceptable means or route, including (for example) by oral, parenteral (e.g., intravenous), inhaled, or topical administration.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal, such as a human). A subject to be treated according to the methods described herein may be one who has been diagnosed with a disease, e.g., a subject diagnosed with an inflammatory disease or lupus, or one at risk of developing the condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

As used herein, the term “therapeutically effective amount” is intended to include an amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 that is effective when administered alone or in combination to inhibit IL-23, IL-12 and/or IFNα function and/or treat diseases. The methods of treating IL-23-, IL-12 and/or IFNα-associated conditions may comprise administering compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 alone or in combination with each other and/or other suitable therapeutic agents useful in treating such conditions. Accordingly, “therapeutically effective amount” is also intended to include an amount of the combination of compounds claimed that is effective to inhibit IL-23, IL-12 and/or IFNα function and/or treat diseases associated with IL-23, IL-12 and/or IFNα.

As used herein, the term “chemotherapeutic agent” includes any other pharmaceutically active compound that can be used in conjunction with the disclosed TYK2 inhibitors.

As used herein, the terms “IL-23-, IL-12- and/or IFNα-associated condition” or “IL-23-, IL-12- and/or IFNα-associated disease or disorder” are intended to encompass all of the conditions identified above as if repeated at length, as well as any other condition that is affected by IL-23, IL-12 and/or IFNα.

The present disclosure further relates to the use of one or more of the TYK2 inhibitors disclosed herein for making a medicament for treating asthma.

The present disclosure further relates to the use of one or more of the TYK2 inhibitors disclosed herein for making a medicament for treating an inflammatory bowel disease.

The present disclosure further relates to the use of one or more of the TYK2 inhibitors disclosed herein for making a medicament for treating Crohn’s disease.

The present disclosure further relates to the use of one or more of the TYK2 inhibitors disclosed herein for making a medicament for treating ulcerative colitis.

The present disclosure further relates to the use of one or more of the TYK2 inhibitors disclosed herein for making a medicament for treating rheumatoid arthritis.

The present disclosure further relates to the use of one or more of the TYK2 inhibitors disclosed herein for making a medicament for treating psoriasis.

The present disclosure further relates to the use of one or more of the TYK2 inhibitors disclosed herein for making a medicament for treating allergic rhinitis.

The present disclosure further relates to the use of one or more of the TYK2 inhibitors disclosed herein for making a medicament for treating atopic or contact dermatitis.

The present disclosure further relates to the use of one or more of the TYK2 inhibitors disclosed herein for making a medicament for treating delayed hypersensitivity reactions.

The present disclosure further relates to the use of one or more of the TYK2 inhibitors disclosed herein for making a medicament for treating lupus.

The present disclosure further relates to the use of one or more of the TYK2 inhibitors disclosed herein for making a medicament for treating multiple sclerosis.

Compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 have utility in treating conditions associated with the modulation of the function of IL12, IL-23 or IFNα, and particularly the selective inhibition of function of IL-23, IL-12 and/or IFNα, by acting on TYK2 to mediate signal transduction. Such conditions include IL-23-, IL-12-, or IFNα-associated diseases in which pathogenic mechanisms are mediated by these cytokines.

In view of their activity as modulators of IL-23-, IL-12 and IFNα-stimulated cellular responses, compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 are useful in treating IL-23-, IL-12- or IFNα-associated diseases including, but not limited to, inflammatory diseases such as Crohn’s disease, ulcerative colitis, asthma, graft versus host disease, allograft rejection, chronic obstructive pulmonary disease; autoimmune diseases such as Graves’ disease, rheumatoid arthritis, systemic lupus erythematosis, cutaneous lupus, lupus nephritis, discoid lupus erythematosus, psoriasis; auto-inflammatory diseases including CAPS, TRAPS, FMF, adult onset stills, systemic onset juvenile idiopathic arthritis, gout, gouty arthritis; metabolic diseases including type 2 diabetes, atherosclerosis, myocardial infarction; destructive bone disorders such as bone resorption disease, osteoarthritis, osteoporosis, multiple myeloma-related bone disorder; proliferative disorders such as acute myelogenous leukemia, chronic myelogenous leukemia; angiogenic disorders such as angiogenic disorders including solid tumors, ocular neovasculization, and infantile haemangiomas; infectious diseases such as sepsis, septic shock, and Shigellosis; neurodegenerative diseases such as Alzheimer’s disease, Parkinson’s disease, cerebral ischemias or neurodegenerative disease caused by traumatic injury, oncologic and viral diseases such as metastatic melanoma, Kaposi’s sarcoma, multiple myeloma, and HIV infection and CMV retinitis, AIDS, respectively.

More particularly, the specific conditions or diseases that may be treated with the inventive compounds include, without limitation, pancreatitis (acute or chronic), asthma, allergies, adult respiratory distress syndrome, chronic obstructive pulmonary disease, glomerulonephritis, rheumatoid arthritis, systemic lupus erythematosis, cutaneous lupus, lupus nephritis, discoid lupus erythematosus, scleroderma, chronic thyroiditis, Graves’ disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn’s disease, psoriasis, graft vs. host disease, inflammatory reaction induced by endotoxin, tuberculosis, atherosclerosis, muscle degeneration, cachexia, psoriatic arthritis, Reiter’s syndrome, gout, traumatic arthritis, rubella arthritis, acute synovitis, pancreatic β-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoidosis, bone resorption disease, allograft rejections, fever and myalgias due to infection, cachexia secondary to infection, keloid formation, scar tissue formation, ulcerative colitis, pyresis, influenza, osteoporosis, osteoarthritis, acute myelogenous leukemia, chronic myelogenous leukemia, metastatic melanoma, Kaposi’s sarcoma, multiple myeloma, sepsis, septic shock, and Shigellosis; Alzheimer’s disease, Parkinson’s disease, cerebral ischemias or neurodegenerative disease caused by traumatic injury; angiogenic disorders including solid tumors, ocular neovasculization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hypoxia [should this be hypoxia], vascular hyperplasia, cardiac and renal reperfusion injury, thrombosis, cardiac hypertrophy, thrombin-induced platelet aggregation, endotoxemia and/or toxic shock syndrome, conditions associated with prostaglandin endoperoxidase syndase-2, and pemphigus vulgaris. Preferred methods of treatment are those wherein the condition is selected from Crohn’s disease, ulcerative colitis, allograft rejection, rheumatoid arthritis, psoriasis, ankylosing spondylitis, psoriatic arthritis, and pemphigus vulgaris. Alternatively preferred methods of treatment are those wherein the condition is selected from ischemia reperfusion injury, including cerebral ischemia reperfusions injury arising from stroke and cardiac ischemia reperfusion injury arising from myocardial infarction. Another preferred method of treatment is one in which the condition is multiple myeloma.

The methods of treating IL-23-, IL-12 and/or IFNα-associated conditions may comprise administering compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 alone or in combination with each other and/or other suitable therapeutic agents useful in treating such conditions.

Examples of such other therapeutic agents include, but are not limited to, corticosteroids, rolipram, calphostin, cytokine-suppressive anti-inflammatory drugs (CSAIDs), Interleukin-10, glucocorticoids, salicylates, nitric oxide, and other immunosuppressants; nuclear translocation inhibitors, such as deoxyspergualin (DSG); non-steroidal anti-inflammatory drugs (NSAIDs) such as ibuprofen, celecoxib and rofecoxib; steroids such as prednisone or dexamethasone; antiviral agents such as abacavir; antiproliferative agents such as methotrexate, leflunomide, FK506 (tacrolimus, PROGRAF®); anti-malarials such as hydroxychloroquine; cytotoxic drugs such as azathiprine and cyclophosphamide; TNF-α inhibitors such as tenidap, anti-TNF antibodies or soluble TNF receptor, and rapamycin (sirolimus or RAPAMUNE®) or derivatives thereof.

The above other therapeutic agents, when employed in combination with the compounds described herein, may be used, for example, in those amounts indicated in the Physicians’ Desk Reference (PDR) or as otherwise determined by one of ordinary skill in the art. In the methods of the present invention, such other therapeutic agent(s) may be administered prior to, simultaneously with, or following the administration of the inventive compounds.

The compounds and compositions described herein may be administered via a variety of routes.

Orally administered preparations can be in the form of solids, liquids, emulsions, suspensions, or gels, or in dosage unit form, for example as tablets or capsules. Tablets can be compounded in combination with other ingredients customarily used, such as tale, vegetable oils, polyols, gums, gelatin, starch, and other carriers The TYK2 inhibitors can be dispersed in or combined with a suitable liquid carrier in solutions, suspensions, or emulsions.

Parenteral compositions intended for injection, either subcutaneously, intramuscularly, or intravenously, can be prepared as liquids or solid forms for solution in liquid prior to injection, or as emulsions. Such preparations are sterile, and liquids to be injected intravenously should be isotonic. Suitable excipients are, for example, water, dextrose, saline, and glycerol.

Administration of pharmaceutically acceptable salts of the substances described herein is included within the scope of the present disclosure. Such salts can be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like. For a helpful discussion of pharmaceutical salts, see S. M. Berge et al., Journal of PharmaceuticalSciences 66.1-19 (1977) the disclosure of which is hereby incorporated by reference.

Substances for injection can be prepared in unit dosage form in ampules, or in multidose containers. The TYK2 inhibitors or compositions comprising one or more TYK2 inhibitors to be delivered, can be present in such forms as suspensions, solutions, or emulsions in oily or preferably aqueous vehicles. Alternatively, a salt of theTYK2 inhibitor can be in lyophilized form for reconstitution, at the time of delivery, with a suitable vehicle, such as sterile pyrogen-free water. Both liquids as well as lyophilized forms that are to be reconstituted will comprise agents, preferably buffers, in amounts necessary to suitably adjust the pH of the injected solution. For any parenteral use, particularly if the formulation is to be administered intravenously, the total concentration of solutes should be controlled to make the preparation isotonic, hypotonic, or weakly hypertonic. Nonionic materials, such as sugars, are preferred for adjusting tonicity, and sucrose is particularly preferred. Any of these forms can further comprise suitable formulary agents, such as search or sugar, glycerol or saline. The compositions per unit dosage, whether liquid or solid, can contain from 0.1% to 99% of polynucleotide material.

### Methods Relating to Inhibition of TYK2 & Modulation of IL-12, IL-23 And/or IFNα

Described herein are compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, or pharmaceutically acceptable isomers, racemates, hydrates, solvates, or salts thereof, useful for the modulation of IL-12, IL-23 and/or IFNαby acting on TYK2 to cause signal transduction inhibition.

Also described herein are methods of treating diseases associated with the modulation of IL-12, IL-23, and/or IFNα, comprising administering to a patient in need of such treatment a therapeutically effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1.

Also described herein are methods for treating proliferative, metabolic, allergic, autoimmune and inflammatory diseases (or use of the compounds of the present invention for the manufacture of a medicament for the treatment of these diseases), comprising administering to a host in need of such treatment a therapeutically effective amount of at least one of the compounds of the present invention.

Also described herein are methods of treating an inflammatory or autoimmune disease (or use of the compounds of the present invention for the manufacture of a medicament for the treatment of these diseases) comprising administering to a patient in need of such treatment a therapeutically-effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1.

Also described herein are methods for treating a disease (or use of the compounds of the present invention for the manufacture of a medicament for the treatment of these diseases), comprising administering to a patient in need of such treatment a therapeutically-effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, wherein the disease is rheumatoid arthritis, multiple sclerosis, systemic lupus erythematosus (SLE), lupus nephritis, cutaneous lupus, inflammatory bowel disease, psoriasis, Crohn’s Disease, psoriatic arthritis, Sjogren’s syndrome, systemic scleroderma, ulcerative colitis, Graves’ disease, discoid lupus erythematosus, adult onset Stills, systemic onset juvenile idiopathic arthritis, gout, gouty arthritis, type 1 diabetes, insulin dependent diabetes mellitus, sepsis, septic shock, Shigellosis, pancreatitis (acute or chronic), glomerulonephritis, autoimmune gastritis, diabetes, autoimmune hemolytic anemia, autoimmune neutropenia, thrombocytopenia, atopic dermatitis, myasthenia gravis, pancreatitis (acute or chronic), ankylosing spondylitis, pemphigus vulgaris, Goodpasture’s disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease, and chronic demyelinating polyneuropathy.

Also described herein are methods of treating an inflammatory or autoimmune disease (or use of the compounds of the present invention for the manufacture of a medicament for the treatment of said diseases), comprising administering to a patient in need of such treatment a therapeutically-effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, wherein the disease is selected from systemic lupus erythematosus (SLE), lupus nephritis, cutaneous lupus, Crohn’s Disease, ulcerative colitis, type 1 diabetes, psoriasis, rheumatoid arthritis, systemic onset juvenile idiopathic arthritis, ankylosing spondylitis, and multiple sclerosis.

Also described herein are methods for treating rheumatoid arthritis (or use of the compounds of the present invention for the manufacture of a medicament for the treatment of rheumatoid arthritis), comprising administering to a patient in need of such treatment a therapeutically-effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1.

Also described herein are methods of treating a condition (or use of the compounds of the present invention for the manufacture of a medicament for the treatment of these conditions) comprising administering to a patient in need of such treatment a therapeutically-effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, wherein the condition is selected from acute myelogenous leukemia, chronic myelogenous leukemia, metastatic melanoma, Kaposi’s sarcoma, multiple myeloma, solid tumors, ocular neovasculization, and infantile haemangiomas, B cell lymphoma, systemic lupus erythematosus (SLE), rheumatoid arthritis, psoriatic arthritis, multiple vasculitides, idiopathic thrombocytopenic purpura (ITP), myasthenia gravis, allergic rhinitis, multiple sclerosis (MS), transplant rejection, Type I diabetes, membranous nephritis, inflammatory bowel disease, autoimmune hemolytic anemia, autoimmune thyroiditis, cold and warm agglutinin diseases, Evans syndrome, hemolytic uremic syndrome/thrombotic thrombocytopenic purpura (HUS/TTP), sarcoidosis, Sjogren’s syndrome, peripheral neuropathies, pemphigus vulgaris and asthma.

Also described herein are methods of treating an IL-12, IL-23, and/or IFNαmediated disease (or use of the compounds of the present invention for the manufacture of a medicament for the treatment of these diseases), comprising administering to a patient in need of such treatment a therapeutically-effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1.

Also described herein are methods of treating an IL-12, IL-23 and/or IFNαmediated disease (or use of the compounds of the present invention for the manufacture of a medicament for the treatment of these diseases), comprising administering to a patient in need of such treatment a therapeutically-effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1, wherein the IL-12, IL-23 and/or IFNαmediated disease is a disease modulated by IL-12, IL-23 and/or IFNα.

Also described herein are methods of treating diseases, comprising administering to a patient in need of such treatment a therapeutically effective amount of a compound having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 in combination with other therapeutic agents.

Also described herein are compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 for use in therapy. In some embodiments, the compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 are selected from exemplified compounds or combinations of exemplified compounds or other embodiments herein.

In other embodiments the compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 have an IC₅₀<1000nM in at least one of the assays described herein.

### Methods Relating to the Treatment of Cancer

As used herein cancer is defined herein as “an abnormal growth of cells which tend to proliferate in an uncontrolled way and, in some cases, to metastasize.” As such, both metastatic and non-metastatic cancers can be treated by the disclosed methods.

Described herein are methods for treating cancer in a human or mammal, comprising administering, to a human or mammal with cancer, an effective amount of one or more compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or pharmaceutically acceptable isomers, racemates, hydrates, solvates, or salts thereof.

Also described herein are methods for treating a human or mammal diagnosed with cancer, comprising administering, to a human or mammal with cancer, an effective amount of one or more compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or pharmaceutically acceptable isomers, racemates, hydrates, solvates, or salts thereof.

Also described herein are methods for treating cancer in a human or mammal, comprising co-administering, to a human or mammal with cancer, an effective amount of one or more compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or pharmaceutically acceptable isomers, racemates, hydrates, solvates or salts thereof, in combination with an effective amount of one or more chemotherapeutic agent or chemotherapeutic compound.

Also described herein are methods for treating a human or mammal diagnosed with cancer, comprising administering, to a human or mammal with cancer, an effective amount of one or more compounds having the structure of any one of Formulas (I), (II), (III), (III i), (III ii), (IV), (IV i), (IV ii), (V), (VI), (VI A), (VI A i), (VI A ii), (VI B), (VI B i), or (VI B ii) or of Table 1 or pharmaceutically acceptable isomers, racemates, hydrates, solvates or salts thereof, in combination with an effective amount of one or more chemotherapeutic agent or chemotherapeutic compound.

The following are non-limiting examples of malignant and non-malignant cancers. Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytoma/Malignant Glioma; Craniopharyngioma; Ependymoblastoma; Ependymoma; Medulloblastoma; Medulloepithelioma; Pineal Parenchymal Tumors of Intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma; Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; Intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocytoma of Bone and Osteosarcoma; Medulloblastoma; Melanoma; Melanoma, Intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis Fungoides; Myelodysplastic Syndromes; Myelodysplastic/Myelo-proliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Cell Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

### Methods for Making a Compound of Formula (I)

A compound having the structure of any one of Formulas (I) (II), (III), (III-i), (III-ii), (IV), (IV-i), (IV-ii), (V), (VI), (VI-A), (VI-A-i-), (VI-A-ii), (VI-B), (VI-B-i), or (VI-B-ii), or having the structure of a compound listed in Table 1, may be synthesized using standard synthetic techniques known to those of skill in the art.

To this end, the reactions, processes and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed.

Unless otherwise indicated, conventional methods of mass spectroscopy (MS), liquid chromatography-mass spectroscopy (LCMS), NMR, HPLC, protein chemistry, biochemistry, recombinant DNA techniques, and pharmacology are employed. Compounds are prepared using standard organic chemistry techniques such as those described in, for example, March’s Advanced Organic Chemistry, 7th Edition, John Wiley and Sons, Inc (2013). Alternate reaction conditions for the synthetic transformations described herein may be employed such as variation of solvent, reaction temperature, reaction time, as well as different chemical reagents and other reaction conditions. As necessary, the use of appropriate protecting groups may be required. The incorporation and cleavage of such groups may be carried out using standard methods described in Peter G. M. Wuts and Theodora W. Green, Protecting Groups in Organic Synthesis, 4th Edition, Wiley-Interscience. (2006). All starting materials and reagents are commercially available or readily prepared.

### Preparation of Pyridine Compounds of Formula I

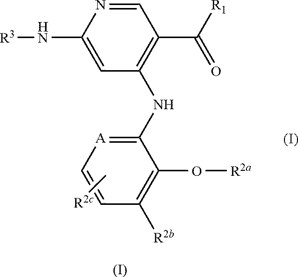

Compounds of Formula (I) may be prepared from known or readily prepared starting materials, following methods known to one skilled in the art of organic synthesis. Methods useful for making the Compounds of Formula (I) are set forth in the Examples below and generalized in Schemes 1, 2, 3, and 4 below. Alternative synthetic pathways and analogous structures will be apparent to those skilled in the art of organic synthesis.

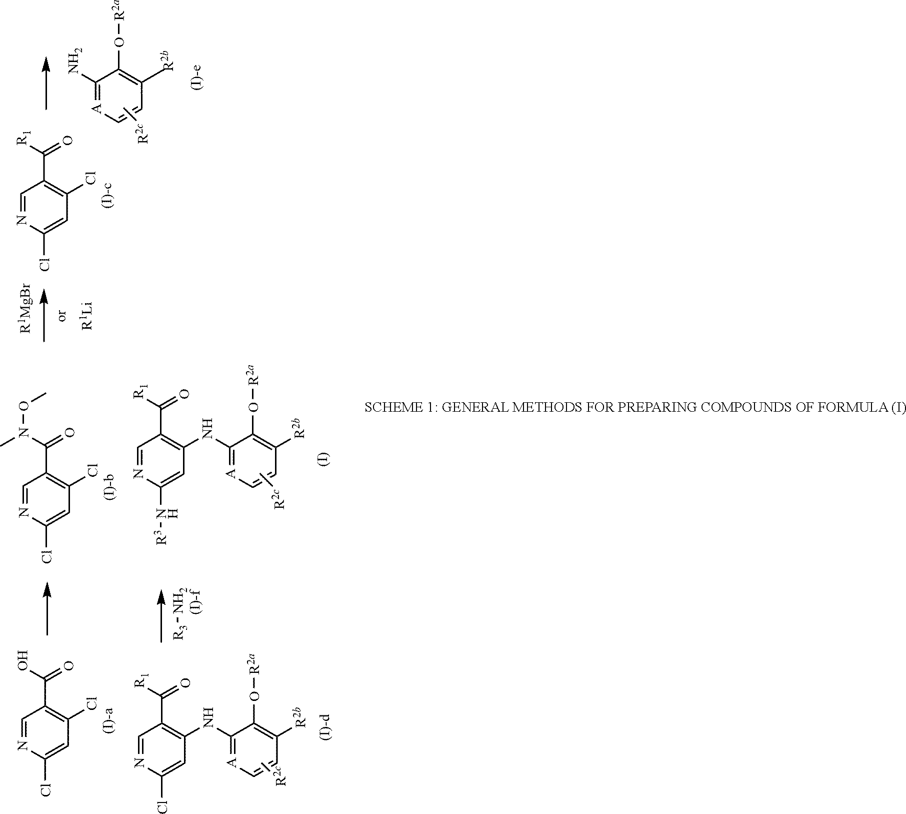

Scheme 1 illustrates general methods for preparing substituted pyridines. The carboxylic acid (I)-a, containing di-halo substitution such as the di-chloro, may be converted to the Weinreb amide (I)-b by conversion to the acid chloride using oxalyl chloride, followed by reaction with N,O-dimethylhydroxylamine. Addition of an R¹ organometallic species, such as a Grignard or organolithium reagent, to (I)-b affords ketone (I)-c. Selective addition of optionally substituted anilines or aminoheterocycles (I)-e can be achieved using an acid catalyzed (such as concentrated hydrochloric acid) SNAr reaction to provide mono-amino substituted pyridine (I)-d. Alternatively, monosubstitution can be achieved via a base mediated reaction using, for example, lithium bis(trimethylsilyl)amide. A palladium-mediated Buchwald coupling of (I)-d with substituted amino-heterocycles or substituted primary amides (I)-f affords Compounds of Formula (I).

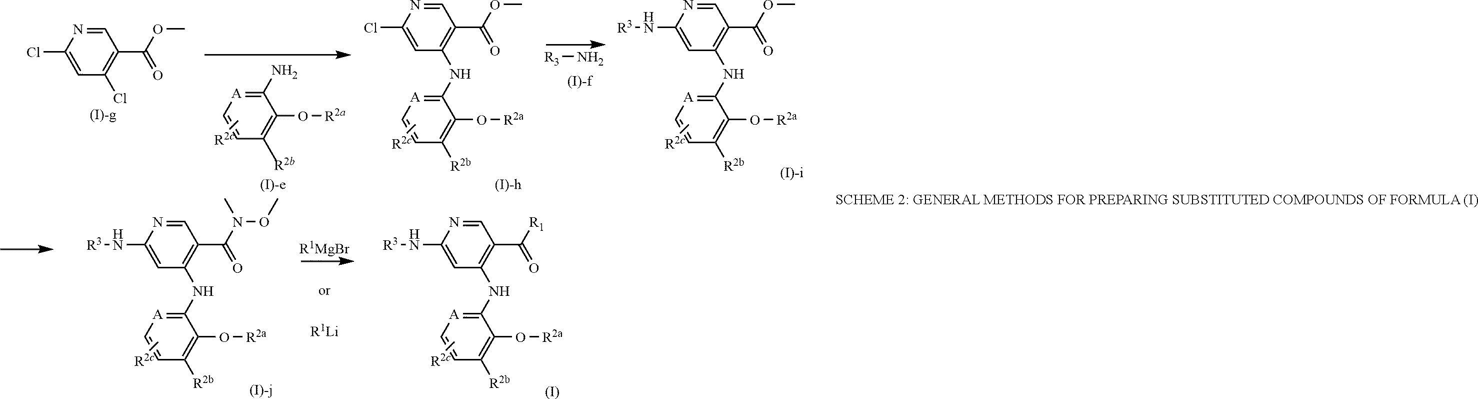

Scheme 2 illustrates general methods for preparing substituted pyridines. The ester (I)-g can undergo substitution at the o-chloro to the ester with various anilines or aminoheterocycles (I)-e using either an acid catalyzed (using, for example, concentrated hydrochloric acid) or a base promoted (using, for example, lithium bis(trimethylsilyl)amide) SNAr reaction to provide mono-amino substituted pyridine (I)-h. This is followed by a palladium-mediated Buchwald coupling with substituted amino-heterocycles or substituted primary amides (I)-f to afford the optionally substituted pyridine (I)-i. Conversion of (I)-i to the Weinreb amide (I)₋j can be achieved by hydrolysis (with for example an aqueous solution of sodium hydroxide in methanol), conversion to the acid chloride (using, for example, oxalyl chloride) and then reaction with N,O-dimethylhydroxylamine. Reaction of (I)₋j with an R¹ organometallic reagent (such as a Grignard or organolithium reagent) affords Compounds of Formula (I).

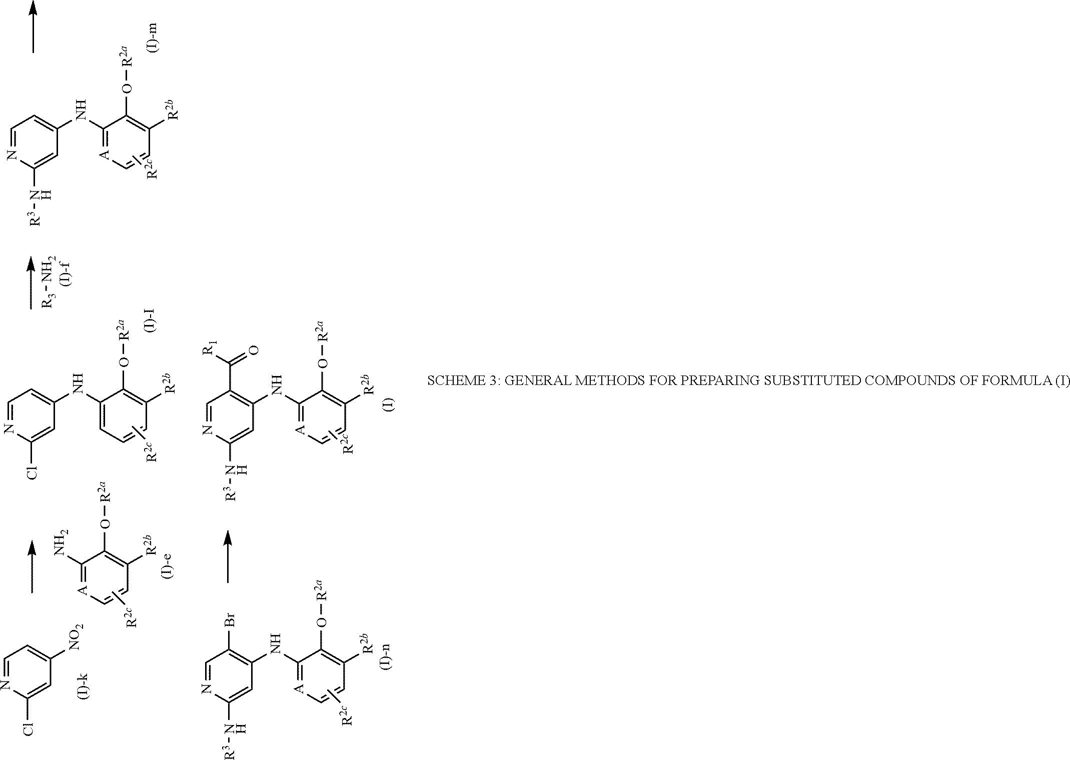

Scheme 3 illustrates general methods for preparing substituted pyridines. The para-nitro o-halopyridine (I)-k can be displaced with optionally substituted anilines or aminoheterocycles (I)-e using a base mediated SNAr reaction (such as sodium hydride in N,N-dimethylformamide). Palladium-mediated Buchwald coupling of (I)-I with amino-heterocycles or substituted primary amines (I)-f affords the optionally substituted pyridine (I)-m. Bromination is achieved using a brominating reagent such as N-bromosuccinimide to afford (I)-n. A palladium-mediated Heck reaction with an R¹ substituted vinyl ether, followed by acid hydrolysis affords Compounds of Formula (I).

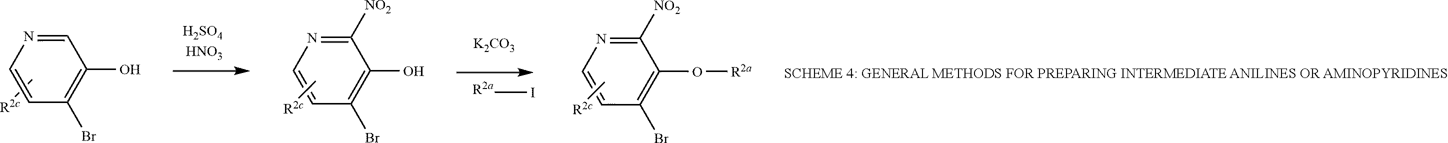

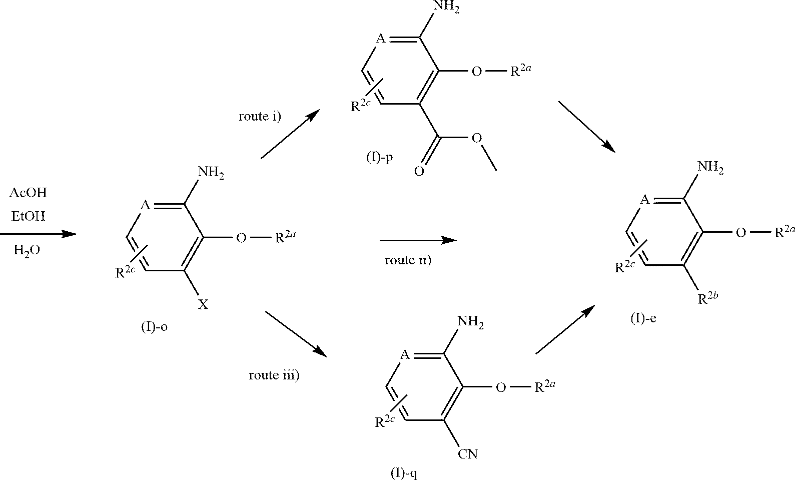

Scheme 4 illustrates general methods for preparing intermediate anilines or aminopyridines. X = halo-substituted aminopyridine starting material (I)-o may be prepared as previously described (see for example WO20191831860), according to the general route shown above. The X = halo-substituted (I)-o can be converted to the ester (I)-p via a palladium mediated carboxylation. Hydrolysis of (I)-p (with, for example, sodium hydroxide in methanol) followed by amide bond formation (with, for example, chloro-N,N,N,N-tetramethyl formamidinium hexafluorophosphate and 1-methylimidazole in acetonitrile), affords intermediate (I)-e, where R^{2b} is an amide (see route i). A palladium mediated Suzuki reaction of (I)-o with a suitable R^{2b} derivative, affords intermediate (I)-e, where R^{2a} is a heteroaryl (see route ii). Alternatively, X = halo-substituted (I)-o can be converted to the nitrile (I)-q (with, for example, tetrakistriphenylphosphine palladium(0), zinc(II)cyanide in N,N-dimethylformamide). Nucleophilic substitution of nitrile (I)-q (with, for example, hydroxylamine), followed by cyclization (with, for example, an acid chloride) affords intermediate (I)-e, where R^{2b} is a heteroaryl (see route iii).

### EXAMPLES

The following examples are provided for illustrative purposes only and not to limit the scope of the claims provided herein. While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1

Preparation of N-(5-Acetyl-4-((2-methoxy-3-(1-methyl-1H-1,2,4-triazol-3-yl)phenyl) amino)pyridin-2-yl) cyclopropanecarboxamide (Compound 1A)

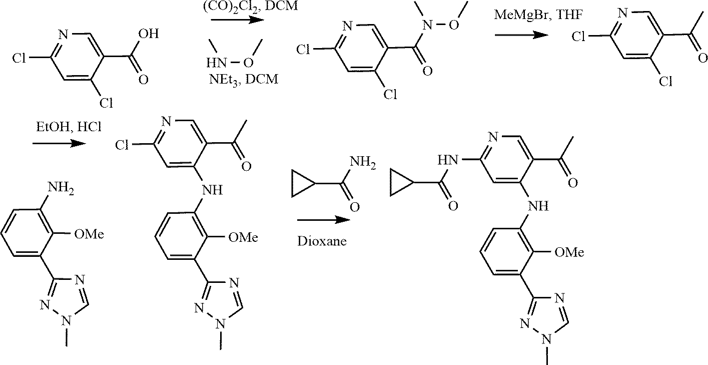

### Step 1: 4,6-Dichloro-N-methoxy-N-methylnicotinamide

Oxalyl chloride (4.53 mL, 52.1 mmol) was added to a solution of 4,6-dichloronicotinic acid (10 g, 52.1 mmol) in dichloromethane (100 mL) and N,N-dimethylformamide (2 mL) at 0° C. under nitrogen. The resulting suspension was stirred for 10 min at 0° C. and then allowed to warm ambient temperature and stirred overnight. The reaction mixture was concentrated under vacuum and the residue resuspended in dichloromethane (100 mL) and added to an ice-bath-cooled mixture of N,O-dimethylhydroxylamine hydrochloride (4.8 g, 78 mmol) and triethylamine (25.4 mL, 182 mmol) in dichloromethane (100 mL). The mixture was stirred at ambient temperature for 4 hours. The resulting solution was diluted with sat. NaHCO₃₍aq) and dichloromethane. The organic layer was separated, washed with water and saturated brine, dried over anhydrous sodium sulfate, filtered and concentrated to provide crude 4,6-dichloro-N-methoxy-N-methylnicotinamide (9.5 g, 78%).

### Step 2: 1-(4,6-Dichloropyridin-3-yl)ethan-1-one

To a solution of 4,6-dichloro-N-methoxy-N-methylnicotinamide (500 mg, 2.1 mmol) in tetrahydrofuran (30 mL) at 0° C. under nitrogen was added a 1 M solution of methyl magnesium bromide in tetrahydrofuran (740 uL, 6.38 mmol) dropwise over 10 minutes. The mixture was stirred for 2 hours at 0° C., then quenched with saturated aqueous ammonium chloride (30 ml). Extracted with ethyl acetate (3 × 50 mL) and the organic layers were dried over anhydrous sodium sulfate. The mixture was filtered and concentrated to yield crude 1-(4,6-dichloropyridin-3-yl)ethan-1-one (350 mg, 87%).

### Step 3: 1-(6-Chloro-4-((2-methoxy-3-(1-methyl-1H-1,2,4-triazol-3-yl)phenyl)amino)pyridin-3-yl)ethan-1-one

To a solution of 2-methoxy-3-(1-methyl-1H-1,2,4-triazol-3-yl)aniline (100 mg, 0.49 mmol) and 1-(4,6-dichloropyridin-3-yl)ethan-1-one (93 mg, 0.49 mmol) in ethanol (20 mL) was added concentrated hydrochloric acid (200 uL) and the resulting mixture stirred overnight at 85° C. The solvent was removed in vacuo and the residue purified on a silica gel column eluting with dichloromethane/methanol (10:1). Desired fractions were combined and concentrated to yield 1-(6-chloro-4-((2-methoxy-3-(1-methyl-1H-1,2,4-triazol-3-yl)phenyl)amino)pyridin-3-yl)ethan-1-one (100 mg, 57%).

### Step 4: N-(5-Acetyl-4-((2-methoxy-3-(1-methyl-1H-1,2,4-triazol-3-yl)phenyl) amino)pyridin-2-yl)cyclopropanecarboxamide

In a vial was combined 1-(6-chloro-4-((2-methoxy-3-(1-methyl-1H-1,2,4-triazol-3-yl)phenyl)amino)pyridin-3-yl)ethan-1-one (70 mg, 0.195 mmol),cyclopropanecarboxamide (25 mg, 0.29 mmol), 2-dicyclohexylhosphino-2′,4′,6′-triisopropylbiphenyl (9 mg, 0.02 mmol), (2-dicyclohexylphosphino-2′,4′,6′-triisopropyl-1,1′-biphenyl)[2-(2′-amino-1,1′-biphenyl)]palladium(II) methanesulfonate (17 mg, 0.02 mmol), cesium carbonate (127 mg, 0.39 mmol) and 1,4-dioxane (12 mL). The vial was sealed and heated to 90° C. for 3 hours. The solvents were removed and the residue was purified on a silica gel column eluting with ethyl acetate. The crude product was purified by Prep-HPLC to yield N-(5-acetyl-4-((2-methoxy-3-(1-methyl-1H-1,2,4-triazol-3-yl)phenyl)amino)pyridin-2-yl)cyclopropanecarboxamide (17 mg, 21%) as a solid.

$$\left(\text{ES} m / z\right) : {\left[\text{M+H}\right]}^{+} = 407.30 .$$

¹H NMR (400 MHz, Methanol-d4) δ 8.82 (s, 1H), 8.49 (s, 1H), 8.10 (s, 1H), 7.68 - 7.63 (m, 2H), 7.31 (t, J= 7.9 Hz, 1H), 4.03 (s, 3H), 3.72 (s, 3H), 2.68 (s, 3H), 1.93 - 1.77 (m, 1H), 0.96 (m, 2H), 0.90 (m, 2H).

### Preparation of Compounds 1B-1C

Compounds 1B and 1C were prepared in a similar fashion using ethyl magnesium bromide and cyclopropyl magnesium bromide respectively, in place of methyl magnesium bromide, in STEP 2, as indicated in TABLE 2.

TABLE 2

COMPOUNDS IB AND 1C

| Cmpd. No. | Structure | Alkylating Agent | ¹H NMR | MS (M+H)⁺ |
|---|---|---|---|---|
| 1B | 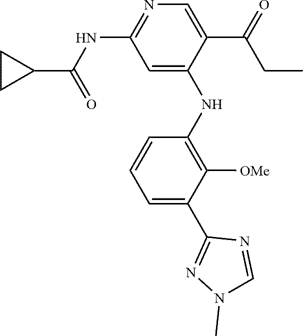 [SMILES: CCC(=O)c1cnc(NC(=O)C2CC2)cc1Nc1cccc(-c2ncn(C)n2)c1OC] | EtMgBr | (300 MHz, Methanol-d4) δ 8.82 (s, 1H), 8.46 (s, 1H), 8.08 (s, 1H), 7.64 (m, 2H), 7.28 (m, 1H), 4.02 (s, 3H), 3.72 (s, 3H), 3.1 (m, 2H), 1.85 (m, 1H), 1.21 (m, 3H), 0.9 (m, 4H). | 421.10 |
| 1C | 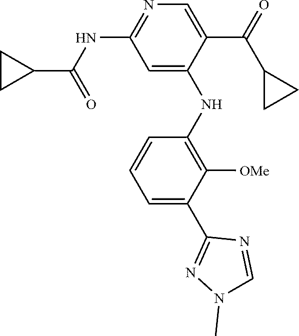 [SMILES: COc1c(Nc2cc(NC(=O)C3CC3)ncc2C(=O)C2CC2)cccc1-c1ncn(C)n1] | 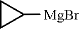 | (300 MHz, Methanol-d4) δ 9.04 (s, 1H), 8.49 (s, 1H), 8.09 (s, 1H), 7.64 (m, 2H), 7.3 (m, 1H), 4.04 (s, 3H), 3.71 (s, 3H), 2.84 (m, 1H), 1.88 (m, 1H), 1.22 (m, 1H), 1.12 (m, 2H), 0.95-0.85 (m, 4H). | 433.25 |

### Preparation of Compound 1D-1P

Compounds 1D-1P were prepared in a similar fashion, using the amide indicated, in place of cyclopropanecarboxamide, in STEP 4, as indicated in TABLE 3.

TABLE 3

COMPOUNDS ID THROUGH 1P

| Cmpd. No. | Structure | Amide | ¹H NMR | MS (M+H)⁺ |
|---|---|---|---|---|
| 1D | 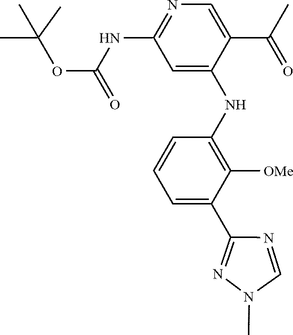 [SMILES: COc1c(Nc2cc(NC(=O)OC(C)(C)C)ncc2C(C)=O)cccc1-c1ncn(C)n1] | 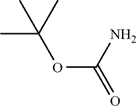 | | |
| 1G | 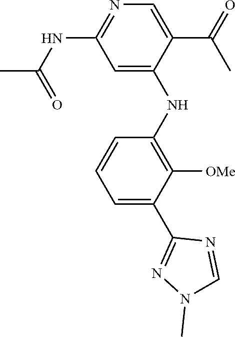 [SMILES: COc1c(Nc2cc(NC(C)=O)ncc2C(C)=O)cccc1-c1ncn(C)n1] | 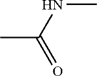 | ¹H NMR (300 MHz, Methanol-d4) δ 8.84 (s, 1H), 8.51 (s, 1H), 8.12 (s, 1H), 7.65 (m, 2H), 7.32 (m, 1H), 4.05 (s, 3H), 3.74 (s, 3H), 2.70 (s, 3H), 2.18 (s, 3H). | 381.10 |
| 1H | 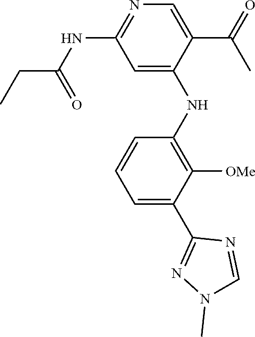 [SMILES: CCC(=O)Nc1cc(Nc2cccc(-c3ncn(C)n3)c2OC)c(C(C)=O)cn1] | 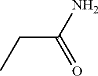 | ¹H NMR (300 MHz, Methanol-d4) δ 8.78 (s, 1H), 8.56 (s, 1H), 7.91 (m, 1H), 7.55 (m, 1H), 7.41 (m, 1H), 6.58 (s, 1H), 4.05 (s, 3H), 3.68 (s, 3H), 2.74 (s, 3H), 2.50 (m, 2H), 1.20 (m, 3H). | 395.15 |
| 1I | 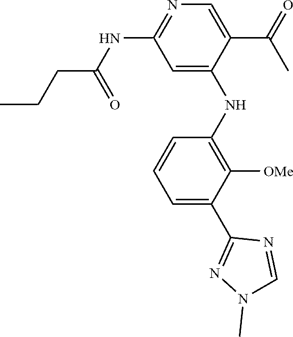 [SMILES: CCCC(=O)Nc1cc(Nc2cccc(-c3ncn(C)n3)c2OC)c(C(C)=O)cn1] | 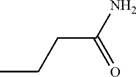 | ¹H NMR (300 MHz, Methanol-d4) δ 8.78 (s, 1H), 8.56 (s, 1H), 7.89 (m, 1H), 7.54 (m, 1H), 7.38 (m, 1H), 6.54 (s, 1H), 4.02 (s, 3H), 3.68 (s, 3H), 2.72 (s, 3H), 2.44 (m, 2H), 1.72 (m, 2H), 0.99 (m, 3H). | 409.15 |
| 1J | 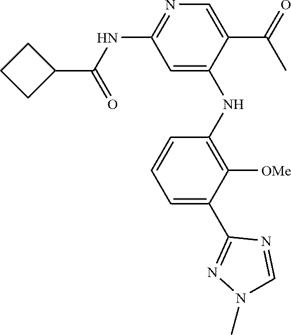 [SMILES: COc1c(Nc2cc(NC(=O)C3CCC3)ncc2C(C)=O)cccc1-c1ncn(C)n1] | 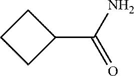 | ¹H NMR (300 MHz, Methanol-d4) δ 8.78 (s, 1H), 8.55 (s, 1H), 7.89 (m, 1H), 7.54 (m, 1H), 7.38 (m, 1H), 6.54 (s, 1H), 4.03 (s, 3H), 3.66 (s, 3H), 3.26 (m, 1H), 2.72 (s, 3H), 2.4-2.2 (m, 4H), 2.1-1.9 (m, 2H). | 421.15 |
| 1K | 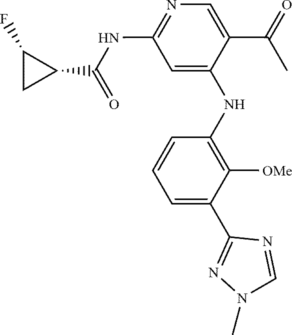 [SMILES: COc1c(Nc2cc(NC(=O)[C@@H]3C[C@@H]3F)ncc2C(C)=O)cccc1-c1ncn(C)n1] | 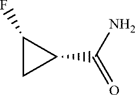 | ¹H NMR (300 MHz, DMSO-d6) δ 11.2 (br s, 1H), 11.12 (br s, 1H), 8.84 (s, 1H), 8.57 (s, 1H), 7.65-7.6 (m, 2H), 7.54 (m, 1H), 7.32 (m, 1H), 5.05-4.82 (m, 1H), 3.96 (s, 3H), 3.72 (s, 3H), 2.68 (s, 3H), 2.18 (m, 1H), 1.7-1.55 (m, 1H), 1.2 (m, 1H). | 425.20 |
| 1L | 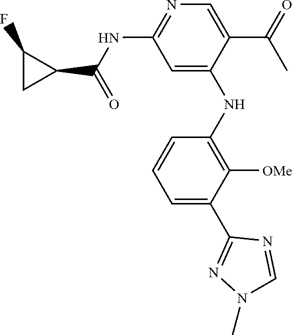 [SMILES: COc1c(Nc2cc(NC(=O)[C@H]3C[C@H]3F)ncc2C(C)=O)cccc1-c1ncn(C)n1] | 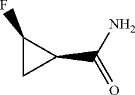 | ¹H NMR (300 MHz, DMSO-d6) δ 11.28 (br s, 1H), 11.14 (br s, 1H), 8.84 (s, 1H), 8.57 (s, 1H), 7.65-7.6 (m, 2H), 7.54 (m, 1H), 7.32 (m, 1H), 5.05-4.82 (m, 1H), 3.96 (s, 3H), 3.72 (s, 3H), 2.68 (s, 3H), 2.18 (m, 1H), 1.7-1.55 (m, 1H), 1.2 (m, 1H). | 425.15 |
| 1M | 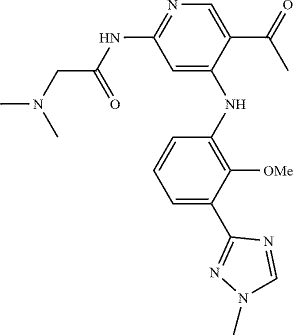 [SMILES: COc1c(Nc2cc(NC(=O)CN(C)C)ncc2C(C)=O)cccc1-c1ncn(C)n1] | 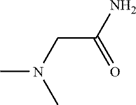 | ¹H NMR (300 MHz, Methanol-d4) δ 8.81 (s, 1H), 8.48 (s, 1H), 8.15 (s, 1H), 7.72-7.64 (m, 2H), 7.28 (m, 1H), 4.04 (s, 3H), 3.72 (s, 3H), 3.14 (s, 2H), 2.68 (s, 3H), 2.37 (s, 6H). | 424.25 |
| 1N | 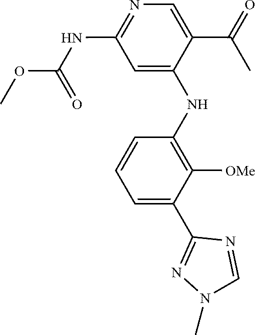 [SMILES: COC(=O)Nc1cc(Nc2cccc(-c3ncn(C)n3)c2OC)c(C(C)=O)cn1] | 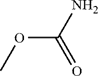 | ¹H NMR (300 MHz, DMSO-d6) δ 11.05 (br s, 1H), 10.42 (br s, 1H), 8.82 (s, 1H), 8.58 (s, 1H), 7.78 (s, 1H), 7.68 (m, 1H), 7.56 (m, 1H), 7.30 (m, 1H), 3.98 (s, 3H), 3.73 (s, 3H), 3.66 (s, 3H), 2.72 (s, 3H), 2.65 (s, 3H). | 397.10 |
| 1O | 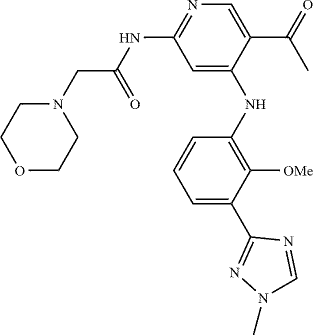 [SMILES: COc1c(Nc2cc(NC(=O)CN3CCOCC3)ncc2C(C)=O)cccc1-c1ncn(C)n1] | 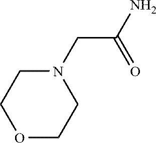 | ¹H NMR (300 MHz, Methanol-d4) δ 8.82 (s, 1H), 8.48 (s, 1H), 8.15 (s, 1H), 7.68 (m, 2H), 7.32 (m, 1H), 4.02 (s, 3H), 3.76 (m, 4H), 3.72 (s, 3H), 3.18 (s, 2H), 2.66 (s, 3H), 2.60 (m, 4H). | 466.20 |
| 1P | 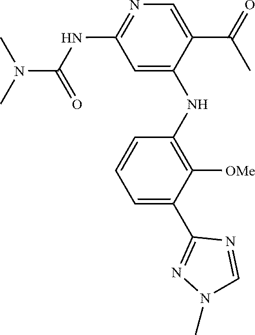 [SMILES: COc1c(Nc2cc(NC(=O)N(C)C)ncc2C(C)=O)cccc1-c1ncn(C)n1] | 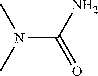 | ¹H NMR (400 MHz, DMSO-d6) δ 11.22 (br s, 1H), 10.05 (br s, 1H), 8.86 (s, 1H), 8.62 (s, 1H), 7.85 (m, 1H), 7.54 (m, 1H), 7.37 (m, 1H), 7.05 (m, 1H), 3.96 (s, 3H), 3.72 (s, 3H), 2.98 (s, 6H), 2.68 (s, 3H). | 410.10 |

### Example 2

Preparation of 1-(6-amino-4-((2-methoxy-3-(1-methyl-1H-1,2,4-triazol-3-yl)phenyl)amino)pyridin-3-yl)ethan-1-one (Compound 2)

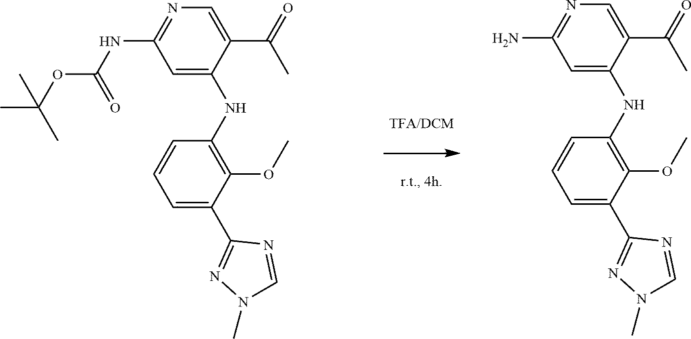

Into a flask, purged and maintained with an inert atmosphere of nitrogen, was placed tert-butyl (5-acetyl-4-((2-methoxy-3-(1-methyl-1H-1,2,4-triazol-3-yl)phenyl)amino)pyridin-2-yl)carbamate (compound 1E, 2 g, 4.6 mmol), dichloromethane (60 ml), and trifluoroacetic acid (60 mL). The resulting solution was stirred for 3 hours at room temperature and then concentrated. The resulting residue was applied onto a silica gel column and eluted with dichloromethane/ methanol (10:1). Desired fractions were combined and concentrated to yield 1-(6-amino-4-((2-methoxy-3-(1-methyl-1H-1,2,4-triazol-3-yl)phenyl)amino)pyridin-3-yl)ethan-1-one (0.91 g, 59%).

$$\text{(ES,} m / z ) : {\text{[M+H]}}^{+} = 339.20$$

¹H NMR (400 MHz, Methanol-d4) δ 8.75 (m, 1H), 8.64 (s, 1H), 7.83 (m, 1H), 7.58 (m, 1H), 7.38 (m, 1H), 6.22 (s, 1H), 4.08 (s, 3H), 3.72 (s, 3H), 2.64 (s, 3H).

### Example 3

Preparation of methyl 6-(cyclopropanecarboxamido)-4-((2-methoxy-3-(1-methyl-1H-1,2,4-triazol-3-yl)phenyl)amino)nicotinate (Compound 3)

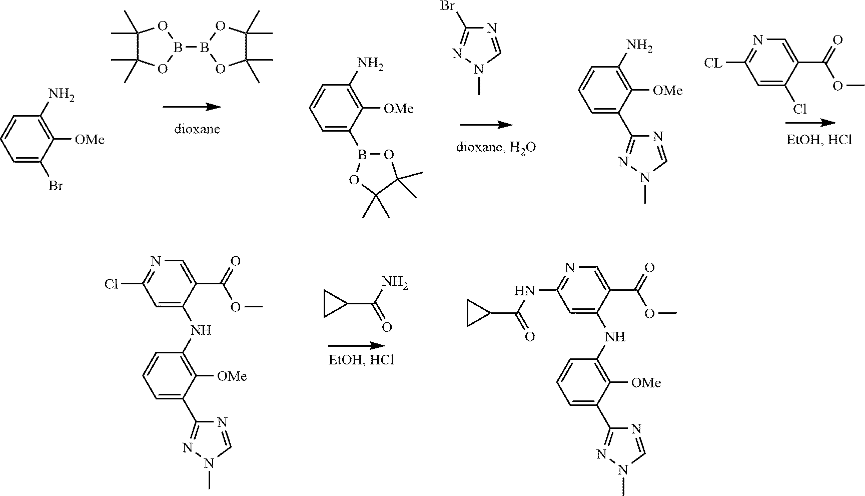

### Step 1: 2-Methoxy-3-(4,4,5,5-tetramethyl-1,3,2-dioxaborolan-2-yl)aniline

To a solution of 3-bromo-2-methoxy-aniline (2.0 g, 9.9 mmol) in 1,4-dioxane (100 mL) was added 4,4,5,5-tetramethyl-2-(4,4,5,5-tetramethyl-1,3,2-dioxaborolan-2-yl)-1,3,2-dioxaborolane (3.0 g, 11.9 mmol), dichloro[1,1′-bis(diphenylphosphino)ferrocene]palladium(II) (724 mg, 0.99 mmol) and potassium acetate (1.9 g, 19.8 mmol) under a nitrogen atmosphere. The resultant mixture was heated at reflux at 100° C. for 4 hours. The mixture was cooled, filtered and the filtrate was evaporated under reduced pressure. The residue was purified by silica gel column eluting with ethyl acetate/petroleum ether (3:1). Desired fractions were combined and concentrated to yield 2-methoxy-3-(4,4,5,5-tetramethyl-1,3,2-dioxaborolan-2-yl)aniline (2.0 g, 80%).

### Step 2: 2-Methoxy-3-(1-methyl-1H-1,2,4-triazol-3-yl)aniline

To a solution of 2-methoxy-3-(4,4,5,5-tetramethyl-1,3,2-dioxaborolan-2-yl)aniline (2.0 g, 8.0 mmol) in 1,4-dioxane (80 mL) and water (20 mL) was added 3-bromo-1-methyl-1H-1,2,4-triazole (1.5 g, 9.6 mmol), 2-dicyclohexylhosphino-2′,4′,6′-triisopropylbiphenyl (760 mg, 1.6 mmol), (2-dicyclohexylphosphino-2′,4′,6′-triisopropyl-1,1′-biphenyl)[2-(2′-amino-1,1′-biphenyl)]palladium(II) methanesulfonate (67 mg, 0.8 mmol) and potassium phosphate tribasic (3.4 g, 16 mmol) under a nitrogen atmosphere. The mixture was stirred at 90° C. for 4 hours. The reaction mixture was cooled, filtered and the filtrate was evaporated under reduced pressure. The residue was purified by silica gel column eluting with 5% methanol in dichloromethane. Desired fractions were combined and concentrated to yield 2-methoxy-3-(1-methyl-1H-1,2,4-triazol-3-yl)aniline (1.4 g, 88%).

### Step 3: Methyl 6-chloro-4-((2-methoxy-3-(1-methyl-1H-1,2,4-triazol-3-yl) phenyl)amino)nicotinate

To a solution of 2-methoxy-3-(1-methyl-1,2,4-triazol-3-yl)aniline (300 mg, 1.47 mmol, 1eq ) and methyl 4,6-dichloronicotinate (303 mg, 1.47 mmol. 1 eq) in ethanol (9.9 mL) was added concentrated hydrochloric acid (100 uL) and stirred overnight at 85° C. The solvent was removed in vacuo, and the resulting residue purified on a silica gel column eluting with dichloromethane/methanol (10:1) to yield methyl 6-chloro-4-((2-methoxy-3-(1-methyl-1H-1,2,4-triazol-3-yl)phenyl)amino)nicotinate (200 mg, 36%).

### step 4: Methyl 6-(cyclopropanecarboxamido)-4-((2-methoxy-3-(1-methyl-1H-1,2,4-triazol-3-yl)phenyl)amino)nicotinate

Into a vial was placed cyclopropanecarboxamide (170 mg, 2.00 mmol), methyl 6-chloro-4-((2-methoxy-3-(1-methyl-1H-1,2,4-triazol-3-yl)phenyl)amino)nicotinate (498 mg, 1.33 mmol), BrettPhos Pd G3 (121 mg, 0.13 mmol), BrettPhos (71 mg, 0.13 mmol), cesium carbonate (868 mg, 2.66 mmol) and 1,4-dioxane (20 mL). The resulting solution was stirred under nitrogen at 90° C. for 2 hours. The solvent was evaporated under reduced pressure, and the resulting residue purified on a silica gel column, eluting with ethyl acetate/petroleum ether (2:1). The product was further purified by Prep-HPLC to yield methyl 6-(cyclopropanecarboxamido)-4-((2-methoxy-3-(1-methyl-1H-1,2,4-triazol-3-yl)phenyl)amino)nicotinate (27.9 mg, 15%).

$$\text{(ES,} m / z ) : {\text{[M+H]}}^{+} 423.15.$$

¹H-NMR (methanol-d4, 400 MHz): 8.77 (s, 1H), 8.51 (s, 1H), 8.12 (s, 1H), 7.70-7.60 (m, 2H), 7.32 (m, 1H), 4.05 (s, 3H), 3.96 (s, 3H), 3.74 (s, 3H), 1.88 (m, 1H), 0.96 (m, 2H), 0.88 (m, 2H).

### Example 4

Preparation of 3-((5-acetyl-2-(cyclopropanecarboxamido)pyridin-4-yl)amino)-2-methoxybenzoic acid (Compound 4A)

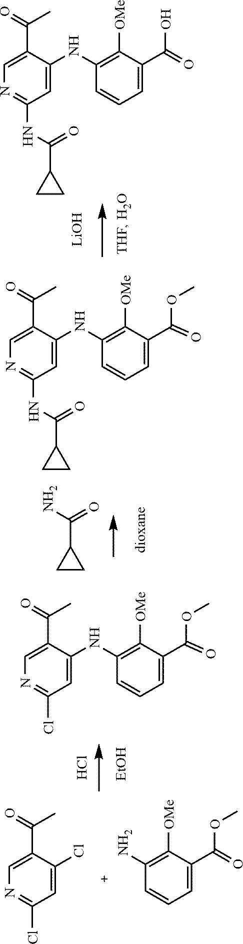

### Step 1: Methyl 3-((5-acetyl-2-chloropyridin-4-yl)amino)-2-methoxybenzoate

A mixture of 1-(4,6-dichloropyridin-3-yl)ethan-1-one (1.0 g, 5.3 mmol) and methyl 3-amino-2-methoxybenzoate (0.95 g, 5.3 mmol) in ethanol (20 mL) and concentrated hydrochloric acid (0.20 mL) was stirred for 4 hours at 80° C. under a nitrogen atmosphere. The resulting mixture was concentrated under reduced pressure, then purified by silica gel column chromatography eluted with hexane/ ethyl acetate (1:1) to yield methyl 3-((5-acetyl-2-chloropyridin-4-yl)amino)-2-methoxybenzoate (1.2 g, 68%) as a solid.

### Step 2: Methyl 3-((5-acetyl-2-(cyclopropanecarboxamido)pyridin-4-yl)amino)-2-methoxybenzoate

A mixture of methyl 3-((5-acetyl-2-chloropyridin-4-yl)amino)-2-methoxybenzoate methoxybenzoate (1.0 g, 3 mmol), cyclopropanecarboxamide (0.38 g, 4.5 mmol), 2-(dicyclohexylphosphino)3,6-dimethoxy-2′,4′,6′-triisopropyl-1,1′-biphenyl (0.32 g, 0.6 mmol), (2-dicyclohexylphosphino-2′,4′,6′-triisopropyl-1,1′-biphenyl)[2-(2′-amino-1,1′-biphenyl)]palladium(II) methanesulfonate (0.27 g, 0.3 mmol) and cesium carbonate (2.9 g, 9 mmol) in 1,4-dioxane (20 mL) was stirred for 3 hours at 90° C. under nitrogen atmosphere. The resulting mixture was concentrated under reduced pressure. The residue was purified by silica gel column chromatography eluting with 10% methanol in dichloromethane. Desired fractions were combined and concentrated to yield methyl 3-((5-acetyl-2-(cyclopropanecarboxamido)pyridin-4-yl)amino)-2-methoxybenzoate (600 mg, 52%) as a solid.

### Step 3: 3-((5-Acetyl-2-(cyclopropanecarboxamido)pyridin-4-yl)amino)-2-methoxybenzoic acid

Lithium hydroxide (55 mg, 1.3 mmol) was added to methyl 3-((5-acetyl-2-(cyclopropanecarbox amido)pyridin-4-yl)amino)-2-methoxybenzoate (100 mg, 0.26 mmol) in tetrahydrofuran (30 mL) and water (10 mL) and the mixture stirred at room temperature overnight. The mixture was concentrated and purified by Prep-HPLC. Combined and concentrated desired fractions to yield 3-((5-acetyl-2-(cyclopropanecarboxamido)pyridin-4-yl)amino)-2-methoxybenzoic acid (29.8 mg, 31%) as the trifluoroacetate salt.

$$\text{(ES,} m / z ) : \text{[M+H]+=370} \text{.1} \text{.}$$

¹H NMR (400 MHz, DMSO-d6) δ 11.04 (s, 1H), 11.01 (s, 1H), 8.85 (s, 1H), 7.85 (s, 1H), 7.64 (dd, J = 8.0, 1.8 Hz, 1H), 7.53 (dd, J = 7.8, 1.6 Hz, 1H), 7.27 (t, J= 7.9 Hz, 1H), 3.73 (s, 3H), 2.66 (s, 3H), 2.05-1.96 (m, 1H), 0.85-0.78 (m, 4H).

### Preparation of Compounds 4B-4E

Compounds 4B-4E were prepared in a similar fashion, according to STEPS 1 and 2, using 2-methoxyaniline, 3-amino-2-methoxybenzonitrile, 2-amino-N-methylbenzamide and 2-amino-N-methylbenzamide in place of methyl 3-amino-2-methoxybenzoate, in STEP 1, as indicated in TABLE 4.

TABLE 4

COMPOUNDS 4B THROUGH 4E

| Cmpd. No. | Structure | Aniline | ¹H NMR | MS (M+H)⁺ |
|---|---|---|---|---|
| 4B | 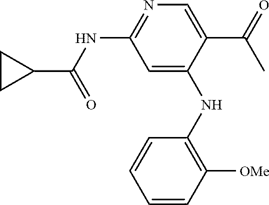 [SMILES: COc1ccccc1Nc1cc(NC(=O)C2CC2)ncc1C(C)=O] | 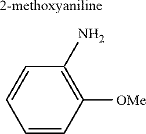 | ¹H NMR (400 MHz, DMSO-d6) δ 10.85 (br s, 1H), 10.76 (br s, 1H), 8.8 (s, 1H), 7.8 (m, 1H), 7.41 (m, 1H), 7.18 (m, 2H), 7.0 (m, 1H), 3.84 (s, 3H), 2.64 (s, 3H), 2.02 (m, 1H), 0.78 (m, 4H). | 326.15 |
| 4C | 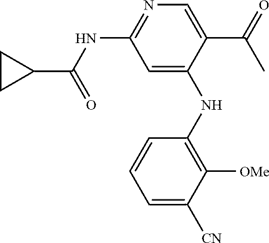 [SMILES: COc1c(C#N)cccc1Nc1cc(NC(=O)C2CC2)ncc1C(C)=O] | 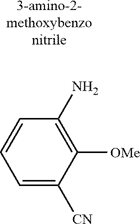 | ¹H NMR (300 MHz, Methanol-d4) δ 8.79 (s, 1H), 7.74 (m, 2H), 7.42 (m, 1H), 6.78 (s, 1H), 4.01 (s, 3H), 2.72 (s, 3H), 1.8 (m, 1H), 1.11.0 (m, 4H). | 349.05 |
| 4D | 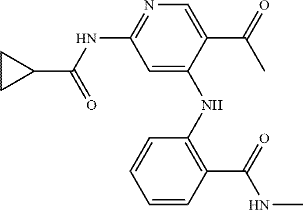 [SMILES: CNC(=O)c1ccccc1Nc1cc(NC(=O)C2CC2)ncc1C(C)=O] | 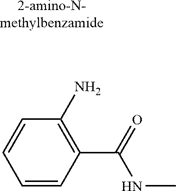 | ¹H NMR (300 MHz, DMSO-d6) δ 11.4 (br s, 1H), 10.84 (br s, 1H), 8.79 (s, 1H), 8.45 (m, 1H), 8.02 (s, 1H), 7.5 (m, 3H), 7.2 (m, 1H), 2.73 (m, 3H), 2.6 (s, 3H), 2.0 (m, 1H), 0.78 (m, 4H). | 353.10 |
| 4E | 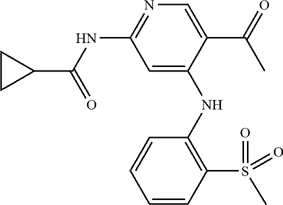 [SMILES: CC(=O)c1cnc(NC(=O)C2CC2)cc1Nc1ccccc1S(C)(=O)=O] | 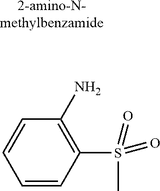 | ¹H NMR (400 MHz, Methanol-d4) δ 8.83 (s, 1H), 8.04 (m, 2H), 7.74 (m, 2H), 7.44 (m, 1H), 4.05 (s, 3H), 3.04 (s, 3H), 2.63 (s, 3H) 1.85 (m, 1H), 0.88 (m, 4H). | 374.05 |

### Example 5

Preparation of 3-((5-acetyl-2-(cyclopropanecarboxamido)pyridin-4-yl)amino)-2-methoxy-N-methylbenzamide (Compound 5A)

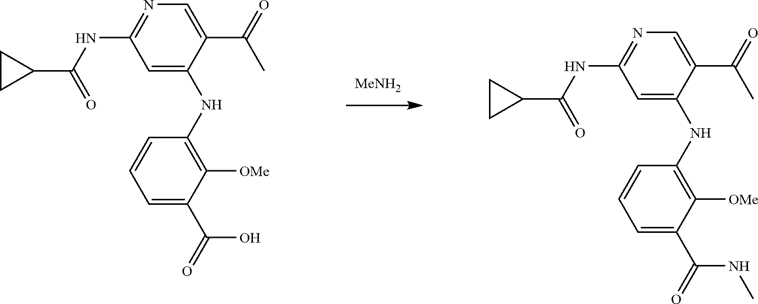 [SMILES: "MeNH2" ; COc1c(Nc2cc(NC(=O)C3CC3)ncc2C(C)=O)cccc1C(=O)O ; CNC(=O)c1cccc(Nc2cc(NC(=O)C3CC3)ncc2C(C)=O)c1OC]

3-((5-acetyl-2-(cyclopropanecarboxamido)pyridin-4-yl)amino)-2-methoxybenzoic acid (120 mg, 0.32 mmol), methylamine hydrochloride (15 mg, 0.49 mmol), chloro-N,N,N′,N′-tetramethylformamidinium hexafluorophosphate (140 mg, 0.49 mmol) and 1-methylimidazole (93 mg, 1.1 mmol) in acetonitrile (20 mL) were stirred for 2 hours at ambient temperature. The mixture was concentrated and purified on a silica gel column eluting with dichloromethane / methanol (10/1). Desired fractions were combined and concentrated to yield 3-((5-acetyl-2-(cyclopropanecarboxamido)pyridin-4-yl)amino)-2-methoxy-N-methylbenzamide (48.9 mg, 39%) as a solid.

$$\text{(ES, m/z): [M+H]+=383} \text{.15} \text{.}$$

¹H NMR (400 MHz, DMSO-d6) δ 11.17 (s, 1H), 11.04 (s, 1H), 8.84 (s, 1H), 8.27 (q, J = 4.6 Hz, 1H), 7.74 (s, 1H), 7.55 (dd, J = 7.9, 1.8 Hz, 1H), 7.37 (dd, J = 7.7, 1.6 Hz, 1H), 7.26 (t, J= 7.8 Hz, 1H), 3.70 (s, 3H), 2.86 - 2.77 (m, 3H), 2.66 (s, 3H), 2.05 - 1.90 (m, 1H), 0.84 (m, 4H).

### Preparation of Compound 5B-5F

Compounds 5B-5F were prepared in a similar fashion to Compound 5A, using an appropriate amine in place of methylamine, as indicated in TABLE 5.

TABLE 5

COMPOUNDS 5B THROUGH 5F

| Cmpd. No. | Structure | Amine | ¹H NMR | MS (M+H)⁺ |
|---|---|---|---|---|
| 5B | 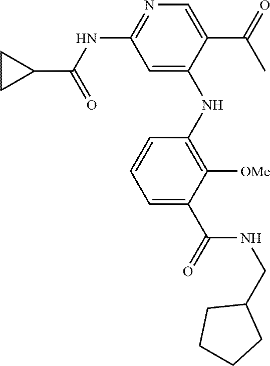 [SMILES: COc1c(Nc2cc(NC(=O)C3CC3)ncc2C(C)=O)cccc1C(=O)NCC1CCCC1] | 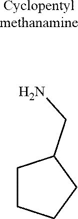 | ¹H NMR (300 MHz, Methanol-d4) δ 8.78 (s, 1H), 7.60 (m, 2H), 7.45 (m, 1H), 7.31 (m, 1H), 3.80 (s, 3H), 3.35 (m, 2H), 2.68 (s, 3H), 2.24 (m, 1H), 1.85 (m, 3H), 1.65 (m, 4H), 1.34 (m, 2H), 1.05-0.9 (m, 4H). | 451.10 |
| 5C | 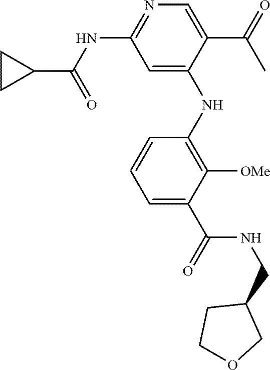 [SMILES: COc1c(Nc2cc(NC(=O)C3CC3)ncc2C(C)=O)cccc1C(=O)NC[C@@H]1CCOC1] | 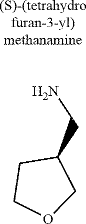 | ¹H NMR (300 MHz, Methanol-d4) δ 8.79 (s, 1H), 7.94 (s, 1H) 7.63 (m, 1H), 7.50 (m, 1H), 7.27 (m, 1H), 3.95-3.77 (m, 6H), 3.62 (m, 1H), 3.44 (m, 2H), 2.66-2.58 (m, 4H), 2.12 (m, 1H), 1.9-1.7 (m, 2H), 0.95-0.85 (m, 4H). | 453.05 |
| 5D | 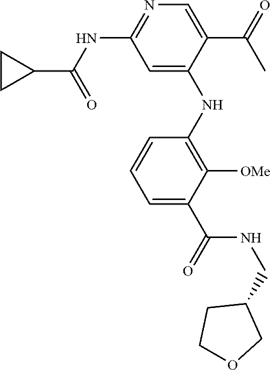 [SMILES: COc1c(Nc2cc(NC(=O)C3CC3)ncc2C(C)=O)cccc1C(=O)NC[C@H]1CCOC1] | 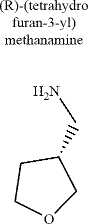 | ¹H NMR (300 MHz, Methanol-d4) δ 8.79 (s, 1H), 7.94 (s, 1H) 7.63 (m, 1H), 7.50 (m, 1H), 7.28 (m, 1H), 3.95-3.75 (m, 6H), 3.62 (m, 1H), 3.44 (m, 2H), 2.66-2.58 (m, 4H), 2.12 (m, 1H), 1.92-1.68 (m, 2H), 0.98-0.85 (m, 4H). | 453.00 |
| 5E | 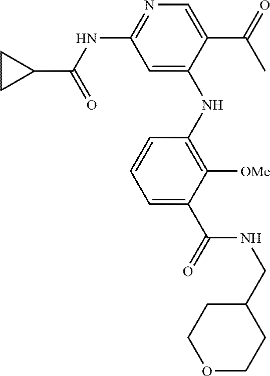 [SMILES: COc1c(Nc2cc(NC(=O)C3CC3)ncc2C(C)=O)cccc1C(=O)NCC1CCOCC1] | 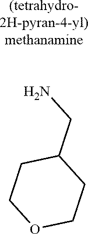 | ¹H NMR (400 MHz, DMSO-d6) δ 10.98 (s, 1H), 10. 95 (s, 1H), 8.75 (s, 1H), 8.35 (m, 1H), 8.0 (m, 1H), 7.54 (m, 1H), 7.3-7.2 (m, 2H), 3.87 (m, 2H), 3.68 (s, 3H), 3.35-3.25 (m, 2H), 3.16 (m, 2H), 2.67 (s, 3H), 2.03 (m, 1H), 1.79 (m, 1H), 1.63 (m, 2H), 1.22 (m, 2H), 0.8 (m, 4H). | 467.10 |
| 5F | 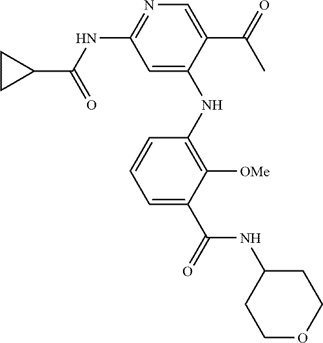 [SMILES: COc1c(Nc2cc(NC(=O)C3CC3)ncc2C(C)=O)cccc1C(=O)NC1CCOCC1] | 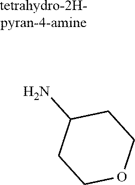 | ¹H NMR (300 MHz, Methanol-d4) δ 8.80 (s, 1H), 7.94 (s, 1H) 7.63 (m, 1H), 7.47 (m, 1H), 7.26 (m, 1H), 4.14 (m, 1H), 4.04-3.94 (m, 2H), 3.77 (s, 3H), 3.55 (m, 2H), 2.66 (s, 3H), 2.0-1.82 (m, 4H), 1.75-1.6 (m, 2H), 0.98-0.85 (m, 4H). | 453.10 |

### Example 6

Preparation of N-(5-acetyl-4-((3-(5-fluoropyridin-2-yl)-2-methoxyphenyl)amino)pyridin-2-yl)cyclopropanecarboxamide (Compound 6A)

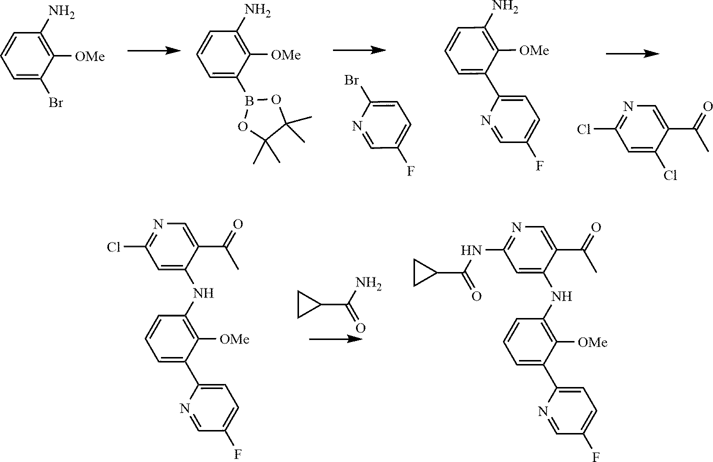

### Step 1: 2-Methoxy-3-(4,4,5,5-tetramethyl-1,3,2-dioxaborolan-2-yl)aniline

To a solution of 3-bromo-2-methoxy-aniline (2.0 g, 9.90 mmol) in 1,4-dioxane (100 mL) under a nitrogen atmosphere was added 4,4,5,5-tetramethyl-2-(4,4,5,5-tetramethyl-1,3,2-dioxaborolan-2-yl)-1,3,2-dioxaborolane (3 g, 12 mmol), [1,1′-bis(diphenylphosphino)ferrocene]dichloropalladium(II) (724 mg, 1 mmol) and potassium acetate (1.94 g, 19.8 mmol). The mixture was refluxed at 100° C. for 4 hours, then cooled, filtered and the filtrate was evaporated under reduced pressure. The residue was purified on a silica gel column eluting with 75% ethyl acetate in petroleum ether. Desired fractions combined and concentrated to yield 2-methoxy-3-(4,4,5,5-tetramethyl-1,3,2-dioxaborolan-2-yl)aniline (2.0 g, 80%).

### Step 2: 3-(5-Fluoropyridin-2-yl)-2-methoxyaniline

Into a round-bottom flask, purged and maintained with an inert atmosphere of nitrogen, was placed 2-methoxy-3-(4,4,5,5-tetramethyl-1,3,2-dioxaborolan-2-yl)aniline (500 mg, 2.0 mmol), 2-bromo-5-fluoropyridine (530 mg, 3 mmol), potassium phosphate tribasic (1.3 g, 6 mmol) in 1,4-dioxane (16 mL) and water (4 mL). [1,1′-bis(di-tert-butylphosphino)ferrocene] dichloropalladium(II) (130 mg, 0.2 mmol) was then added and the mixture stirred for 2 hours at 100° C. Filtered off solids, extracted with ethyl acetate (3 × 50 mL) and concentrated under vacuum. The residue was purified on a silica gel column eluting with 20% ethyl acetate in petroleum ether. The collected fractions were combined and concentrated to yield 3-(5-fluoropyridin-2-yl)-2-methoxyaniline (310 mg, 71%).

### Step 3: 1-(6-Chloro-4-((3-(5-fluoropyridin-2-yl)-2-methoxyphenyl)amino)pyridin-3-yl)ethan-1-one

Into a round-bottom flask, purged and maintained with an inert atmosphere of nitrogen, was placed 3-(5-fluoropyridin-2-yl)-2-methoxyaniline (310 mg, 1.4 mmol), ethanol (10 mL), 1-(4,6-dichloropyridin-3-yl)ethanone (540 mg, 2.8 mmol), and p-toluenesulfonic acid (24 mg, 0.14 mmol). The mixture was stirred overnight at 80° C., then concentrated under vacuum. The residue was purified on a silica gel column with ethyl acetate/petroleum ether (1:2). Collected fractions were combined and concentrated to yield 1-(6-chloro-4-((3-(5-fluoropyridin-2-yl)-2-methoxyphenyl)amino)pyridin-3-yl)ethan-1-one (75 mg, 14%) as a solid.

### Step 4: N-(5-Acetyl-4-((3-(5-fluoropyridin-2-yl)-2-methoxyphenyl)amino)pyridin-2-yl)cyclopropanecarboxamide

Into a vial purged and maintained with an inert atmosphere of nitrogen, was placed 1-(6-chloro-4-((3-(5-fluoropyridin-2-yl)-2-methoxyphenyl)amino)pyridin-3-yl)ethan-1-one (75 mg, 0.20 mmol), cyclopropanecarboxamide (26 mg, 0.30 mmol), 1,4-dioxane (5 mL), cesium carbonate (130 mg, 0.40 mmol), 2-dicyclohexylphosphino-2′,6′-diisopropoxybiphenyl (9 mg, 0.02 mmol), (2-dicyclohexylphosphino-2′,6′-diisopropoxy-1,1′-biphenyl)[2-(2′-amino-1,1′-biphenyl)]palladium(II) methanesulfonate (17 mg, 0.02 mmol). The resulting solution was stirred overnight at 110° C. The solids were removed by filtration and the filtrate extracted with ethyl acetate (3 × 30 mL) and the organic layers combined and concentrated. The residue was purified on a silica gel column eluting with 5% methanol in dichloromethane. The collected fractions were combined and concentrated and repurified by Prep-HPLC. Desired fractions were combined and concentrated to yield N-(5-acetyl-4-((3-(5-fluoropyridin-2-yl)-2-methoxyphenyl)amino)pyridin-2-yl)cyclopropanecarboxamide (25.9 mg (31%) as a solid.

$$\left(\text{ES} \text{m} / \text{z}\right) : \left[\text{M+H}\right] + = 421.15 .$$

¹H NMR (400 MHz, DMSO-d6) δ 11.04 (s, 1H), 10.96 (s, 1H), 8.85 (s, 1H), 8.43 (d, J= 2.5 Hz, 1H), 8.25 - 8.15 (m, 1H), 8.06 (s, 1H), 7.53 (dd, J= 7.7, 1.9 Hz, 1H), 7.36 - 7.24 (m, 3H), 3.3 (s, 3H), 2.66 (s, 3H), 2.02 (m, 1H), 0.85 - 0.77 (m, 4H).

### Preparation of Compounds 6B-6D

Compounds 6B-6D were prepared in a similar fashion using 2-bromo-5-fluoropyridine, 2-bromo-5-fluoropyrimidine and 3-bromo-1-methyl-1H-pyrazole respectively, in place of bromo-5-fluoropyridine, in STEP 2, as indicated in TABLE 6.

TABLE 6

COMPOUNDS 6B THROUGH 6D

| Cmpd. No. | Structure | Aryl bromide | ¹H NMR | MS (M+H)⁺ |
|---|---|---|---|---|
| 6B | 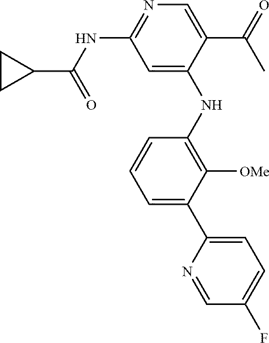 [SMILES: COc1c(Nc2cc(NC(=O)C3CC3)ncc2C(C)=O)cccc1-c1ccc(F)cn1] | 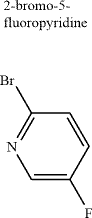 | ¹H NMR (400 MHz, DMSO-d6) δ 11.02 (br s, 1H), 10.96 (br s, 1H), 8.86 (s, 1H), 8.45 (m, 1H), 8.2 (m, 1H), 8.08 (m, 1H), 7.55 (m, 1H), 7.31 (m, 3H), 3.3 (s, 3H), 2.65 (s, 3H), 2.04 (m, 1H), 0.8 (m, 4H). | 421.15 |

| Cmpd. No. | Structure | Aryl bromide | ¹H NMR | MS (M+H)⁺ |
|---|---|---|---|---|
| 6C | 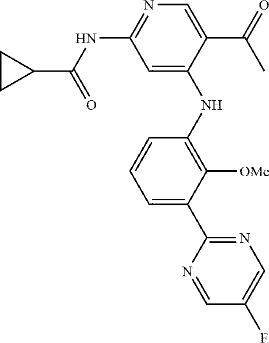 [SMILES: COc1c(Nc2cc(NC(=O)C3CC3)ncc2C(C)=O)cccc1-c1ncc(F)cn1] | 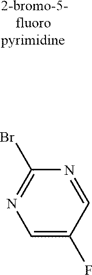 | ¹H NMR (400 MHz, DMSO-d6) δ 11.04 (br s, 1H), 10.96 (br s, 1H), 9.06 (s, 2H), 8.86 (s, 1H), 8.06 (m, 1H), 7.60 (m, 1H), 7.52 (m, 1H), 7.33 (m, 1H), 3.66 (s, 3H), 2.67 (s, 3H), 2.04 (m, 1H), 0.82 (m, 4H). | 422.15 |
| 6D | 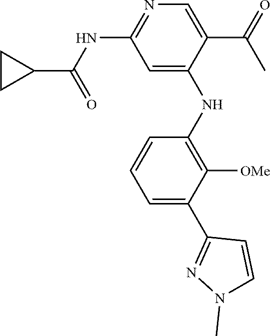 [SMILES: COc1c(Nc2cc(NC(=O)C3CC3)ncc2C(C)=O)cccc1-c1ccn(C)n1] | 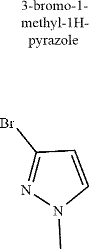 | ¹H NMR (400 MHz, DMSO-d6) δ 11.04 (br s, 1H), 10.95 (br s, 1H), 8.86 (s, 1H), 8.05 (m, 1H), 7.78 (m, 1H), 7.70 (m, 1H), 7.42 (m, 1H), 7.22 (m, 1H), 6.74 (m, 1H), 3.92 (s, 3H), 3.6 (s, 3H), 2.67 (s, 3H), 2.04 (m, 1H), 0.8 (m, 4H). | 406.15 |

### Example 7

Preparation of (6-((5-fluoropyridin-2-yl)amino)-4-((2-methoxy-3-(1-methyl-1H-1,2,4-triazol-3-yl)phenyl)amino)pyridin-3-yl)methanol (Compound 7A)

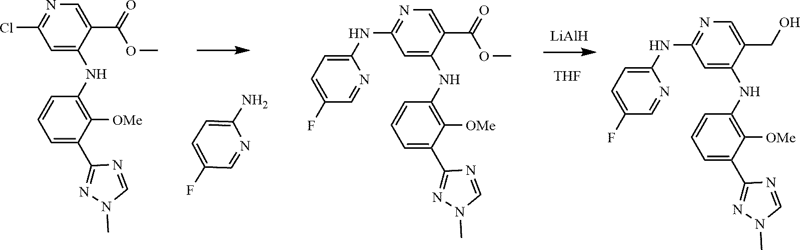

### Step 1: Methyl 6-((5-fluoropyridin-2-yl)amino)-4-((2-methoxy-3-(1-methyl-1H-1,2,4-triazol-3-yl)phenyl)amino)nicotinate

To a solution of methyl 6-chloro-4-[2-methoxy-3-(1-methyl-1,2,4-triazol-3-yl)anilino]pyridine-3-carboxylate (530 mg, 1.4 mmol, prepared as described herein) and 5-fluoropyridin-2-amine (191 mg, 1.7 mmol) in N,N-dimethylformamide (10 mL) was added 2-(dicyclohexylphosphino)3,6-dimethoxy-2′,4′,6′-triisopropyl-1,1′-biphenyl (152 mg, 0.28 mmol), [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate (128 mg, 0.14 mmol), cesium carbonate (924 mg, 2.8 mmol) and then stirred at 100° C. overnight. The solvent was removed under vacuum and the residue was purified by column chromatography eluting with dichloromethane/methanol (20:1) to obtain methyl 6-((5-fluoropyridin-2-yl)amino)-4-((2-methoxy-3-(1-methyl-1H-1,2,4-triazol-3-yl)phenyl)amino)nicotinate (232 mg, 36%).

### Step 2: (6-((5-Fluoropyridin-2-yl)amino)-4-((2-methoxy-3-(1-methyl-1H-1,2,4-triazol-3-yl)phenyl)amino)pyridin-3-yl)methanol

To a solution of methyl 6-((5-fluoropyridin-2-yl)amino)-4-((2-methoxy-3-(1-methyl-1H-1,2,4-triazol-3-yl)phenyl)amino)nicotinate (230 mg, 0.5 mmol) in tetrahydrofuran (10 mL) was slowly added lithium aluminum hydride (117 mg, 3.1 mmol) at 0° C. The reaction was stirred at room temperature for 2 hours, then quenched with saturated ammonium chloride. Extracted with ethyl acetate, washed with brine and dried over anhydrous sodium sulfate. The organic phase was concentrated under vacuum. Purified by Prep-HPLC to yield (6-((5-fluoropyridin-2-yl)amino)-4-((2-methoxy-3-(1-methyl-1H-1,2,4-triazol-3-yl)phenyl)amino)pyridin-3-yl)methanol (12 mg, 6%) as a solid.

$$\left(\text{ES,} m / z\right) : \left[{\text{M+H}}^{+}\right] : = 422.25 .$$

¹H NMR (400 MHz, Methanol-d4) δ 8.62(s,1H), 8.54 (d, J= 15.9 Hz, 1H), 8.19 (d, J = 3.0 Hz, 1H), 7.93 (s, 1H), 7.72 (m, J = 7.8, 1.7 Hz, 1H), 7.61 (m, 2H), 7.35 (t, J = 7.9 Hz, 1H), 7.20 (m, 1H), 6.91 (s, 1H), 4.75 (s, 2H), 4.04 (s, 3H), 3.71 (s, 3H).

### Preparation of Compounds 7B-7U

Compounds 7B-7U were prepared in a similar fashion, starting from 1-(6-chloro-4-((2-methoxy-3-(1-methyl-1H-1,2,4-triazol-3-yl)phenyl)amino)pyridin-3-yl)ethan-1-one in place of methyl 6-chloro-4-((2-methoxy-3-(1-methyl-1H-1,2,4-triazol-3-yl)phenyl)amino)nicotinate, and using the aniline indicatedin TABLE 7 in place of 5-fluoropyridin-2-amine in STEP 1.

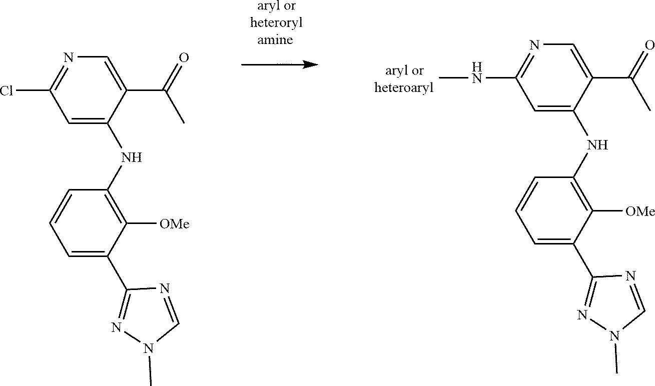

TABLE 7

COMPOUNDS 7B THROUGH 7U

| Cmpd. No. | Structure | Aniline | ¹H NMR | MS (M+H)+ |
|---|---|---|---|---|
| 7B | 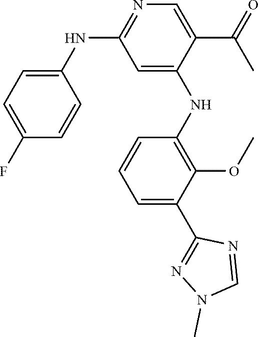 [SMILES: COc1c(Nc2cc(Nc3ccc(F)cc3)ncc2C(C)=O)cccc1-c1ncn(C)n1] | 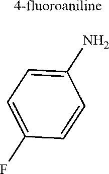 | ¹H NMR (300 MHz, Methanol-d4) δ 8.68 (s, 1H), 8.47 (s, 1H), 7.67 (m, 1H), 7.48 (m, 1H), 7.37 (m, 2H), 7.25 (m, 1H), 7.03 (m, 2H), 6.31 (s, 1H), 4.02 (s, 3H), 3.68 (s, 3H), 2.62 (s, 3H). | 433.10 |
| 7C | 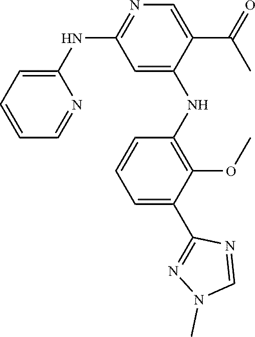 [SMILES: COc1c(Nc2cc(Nc3ccccn3)ncc2C(C)=O)cccc1-c1ncn(C)n1] | 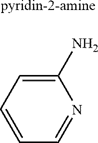 | ¹H NMR (300 MHz, Methanol-d4) δ 8.92 (s, 1H), 8.55 (s, 1H), 8.38 (m, 1H), 7.95-7.85 (m, 2H), 7.58 (m, 1H), 7.4 (m, 1H), 7.22 (m, 1H), 7.06 (m, 1H), 6.48 (s, 1H), 4.04 (s, 3H), 3.71 (s, 3H), 2.74 (s, 3H). | 416.15 |
| 7D | 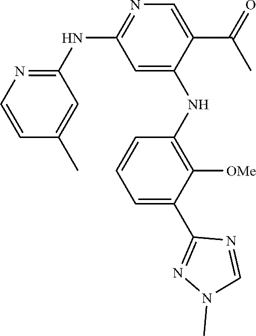 [SMILES: COc1c(Nc2cc(Nc3cc(C)ccn3)ncc2C(C)=O)cccc1-c1ncn(C)n1] | 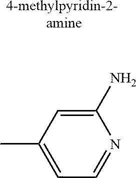 | ¹H NMR (400 MHz, DMSO-d6) δ 11.18 (br s, 1H), 11.06 (br s, 1H), 8.94 (s, 1H), 8.60 (s, 1H), 8.24 (m, 1H), 7.82 (m, 1H), 7.58 (m, 1H), 7.39 (m, 1H), 7.8 (m, 1H), 6.98 (m, 1H), 6.72 (m, 1H), 3.96 (s, 3H), 3.72 (s, 3H), 2.7 (s, 3H), 2.4 (s, 3H). | 430.10 |
| 7E | 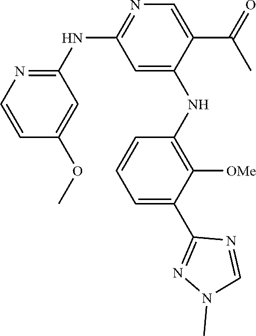 [SMILES: COc1ccnc(Nc2cc(Nc3cccc(-c4ncn(C)n4)c3OC)c(C(C)=O)cn2)c1] | 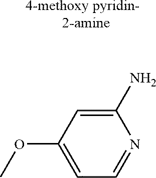 | ¹H NMR (300 MHz, Methanol-d4) δ 8.92 (s, 1H), 8.54 (s, 1H), 8.18 (m, 1H), 7.80 (m, 1H), 7.55 (m, 1H), 7.34 (m, 1H), 6.88 (m, 1H), 6.54 (m, 1H), 6.5 (s, 1H), 4.05 (s, 3H), 3.98 (s, 3H), 3.69 (s, 3H), 2.7 (s, 3H). | 446.20 |
| 7F | 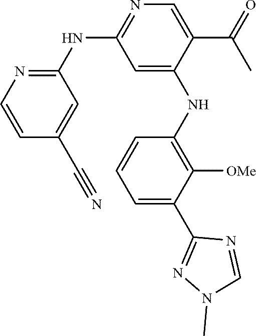 [SMILES: COc1c(Nc2cc(Nc3cc(C#N)ccn3)ncc2C(C)=O)cccc1-c1ncn(C)n1] | 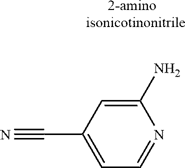 | ¹H NMR (400 MHz, DMSO-d6) δ 11.08 (br s, 1H), 10.44 (br s, 1H), 8.94 (s, 1H), 8.66 (s, 1H), 8.45 (m, 1H), 8.18 (m, 1H), 7.65 (m, 1H), 7.57 (m, 1H), 7.35 (m, 2H), 3.96 (s, 3H), 3.75 (s, 3H), 2.66 (s, 3H). | 441.05 |
| 7G | 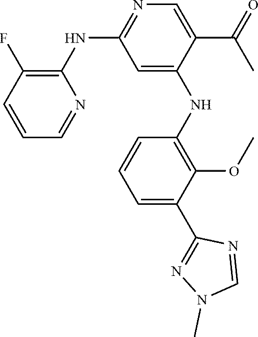 [SMILES: COc1c(Nc2cc(Nc3ncccc3F)ncc2C(C)=O)cccc1-c1ncn(C)n1] | 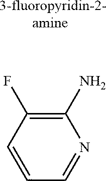 | ¹H NMR (400 MHz, Methanol-d4) δ 8.79 (s, 1H), 8.53 (s, 1H), 8.26 (m, 1H), 8.06 (m, 1H), 7.77 (m, 1H), 7.71 (m, 1H), 7.56 (m, 1H), 7.36 (m, 1H), 7.02 (m, 1H), 4.05 (s, 3H), 3.74 (s, 3H), 2.68 (s, 3H). | 434.10 |
| 7H | 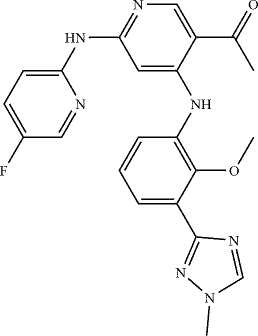 [SMILES: COc1c(Nc2cc(Nc3ccc(F)cn3)ncc2C(C)=O)cccc1-c1ncn(C)n1] | 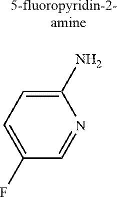 | ¹H NMR (400 MHz, Methanol-d4) δ 8.79 (s, 1H), 8.52 (s, 1H), 8.1 (s, 1H), 7.75-7.65 (m, 3H), 7.54 (m, 2H), 7.35 (m, 1H), 4.06 (s, 3H), 3.74 (s, 3H), 2.66 (s, 3H). | 434.15 |
| 7I | 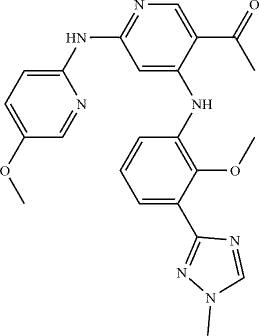 [SMILES: COc1ccc(Nc2cc(Nc3cccc(-c4ncn(C)n4)c3OC)c(C(C)=O)cn2)nc1] | 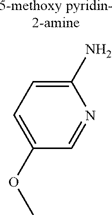 | ¹H NMR (400 MHz, Methanol-d4) δ 8.77 (s, 1H), 8.49 (s, 1H), 7.95 (m, 1H), 7.68 (m, 2H), 7.4-7.25 (m, 4H), 4.05 (s, 3H), 3.85 (s, 3H), 3.73 (s, 3H), 2.65 (s, 3H). | 446.05 |
| 7J | 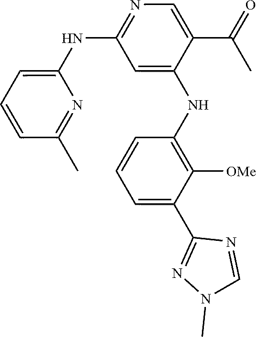 [SMILES: COc1c(Nc2cc(Nc3cccc(C)n3)ncc2C(C)=O)cccc1-c1ncn(C)n1] | 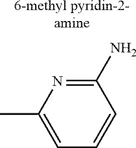 | ¹H NMR (300 MHz, Methanol-d4) δ 9.08 (s, 1H), 8.52 (s, 1H), 7.88-7.78 (m, 2H), 7.59 (m, 1H), 7.38 (m, 1H), 7.10 (m, 1H), 6.86 (m, 1H), 6.51 (s, 1H), 4.05 (s, 3H), 4.02 (s, 3H), 3.7 (s, 3H), 2.71 (s, 3H). | 430.10 |
| 7K | 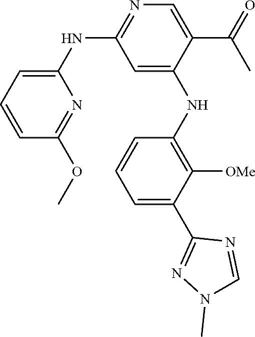 [SMILES: COc1cccc(Nc2cc(Nc3cccc(-c4ncn(C)n4)c3OC)c(C(C)=O)cn2)n1] | 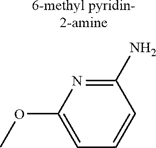 | ¹H NMR (300 MHz, Methanol-d4) δ 9.04 (s, 1H), 8.54 (s, 1H), 7.88 (m, 1H), 7.76 (m, 1H), 7.55 (m, 1H), 7.36 (m, 1H), 6.62-6.52 (m, 3H), 4.05 (s, 3H), 4.02 (s, 3H), 3.7 (s, 3H), 2.71 (s, 3H). | 446.10 |
| 7L | 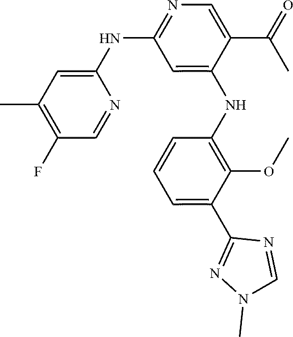 | 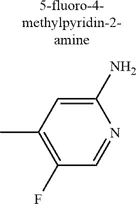 | ¹H NMR (300 MHz, Methanol-d4) δ 8.78 (s, 1H), 8.52 (s, 1H), 7.97 (s, 1H), 7.68 (m, 2H), 7.59 (s, 1H), 7.34 (m, 2H), 4.06 (s, 3H), 3.74 (s, 3H), 2.66 (s, 3H). | 448.15 |
| 7M | 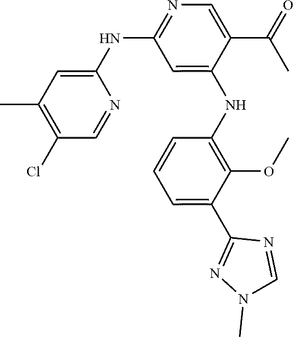 | 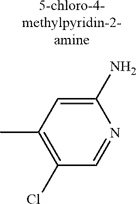 | ¹H NMR (400 MHz, DMSO-d6) δ 11.10 (br s, 1H), 10.08 (br s, 1H), 8.84 (s, 1H), 8.56 (s, 1H), 8.17 (s, 1H), 7.75-7.6 (m, 4H), 7.34 (m, 1H), 3.96 (s, 3H), 3.73 (s, 3H), 2.64 (s, 3H), 2.31 (s, 3H). | 464.25 |
| 7N | 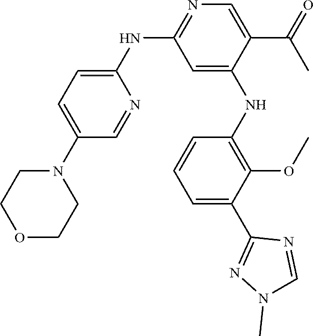 [SMILES: COc1c(Nc2cc(Nc3ccc(N4CCOCC4)cn3)ncc2C(C)=O)cccc1-c1ncn(C)n1] | 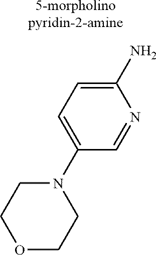 | ¹H NMR (300 MHz, Methanol-d4) δ 8.86 (s, 1H), 8.54 (s, 1H), 8.03 (m, 1H), 7.85 (m, 1H), 7.65-7.55 (m, 2H), 7.37 (m, 1H), 6.98 (m, 1H), 6.42 (s, 1H), 4.04 (s, 3H), 3.85 (m, 4H), 3.71 (s, 3H), 3.18 (m, 4H), 2.71 (s, 3H). | 501.20 |
| 7O | 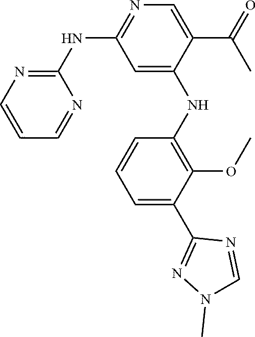 [SMILES: COc1c(Nc2cc(Nc3ncccn3)ncc2C(C)=O)cccc1-c1ncn(C)n1] | 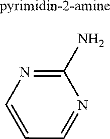 | ¹H NMR (400 MHz, DMSO-d6) δ 11.54 (br s, 1H), 11.22 (br s, 1H), 8.96 (s, 1H), 8.73 (m, 2H), 8.62 (s, 1H), 7.84 (m, 1H), 7.61 (m, 1H), 7.4 (m, 1H), 7.27 (m, 1H), 7.14 (m, 1H), 3.96 (s, 3H), 3.75 (s, 3H), 2.7 (s, 3H). | 417.10 |
| 7P | 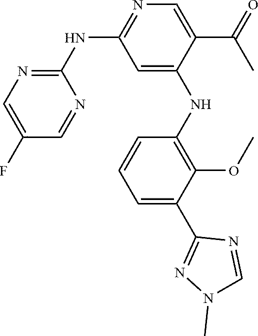 [SMILES: COc1c(Nc2cc(Nc3ncc(F)cn3)ncc2C(C)=O)cccc1-c1ncn(C)n1] | 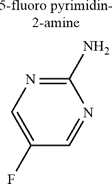 | ¹H NMR (400 MHz, DMSO-d6) δ 11.18 (br s, 1H), 10.38 (br s, 1H), 8.86 (s, 1H), 8.68 (s, 2H), 8.57 (s, 1H), 8.31 (s, 1H), 7.72 (m, 1H), 7.64 (m, 1H), 7.4 (m, 1H), 3.96 (s, 3H), 3.74 (s, 3H), 2.66 (s, 3H). | 435.10 |
| 7Q | 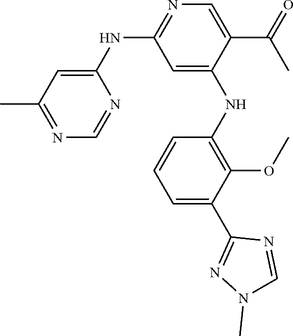 [SMILES: COc1c(Nc2cc(Nc3cc(C)ncn3)ncc2C(C)=O)cccc1-c1ncn(C)n1] | 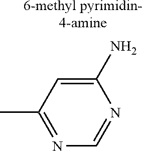 | ¹H NMR (400 MHz, Methanol-d4) δ 8.85 (s, 1H), 8.52 (m, 2H), 7.72 (m, 3H), 7.51 (s, 1H), 7.35 (m, 1H), 4.06 (s, 3H), 3.72 (s, 3H), 2.68 (s, 3H), 2.44 (s, 3H). | 431.10 |
| 7R | 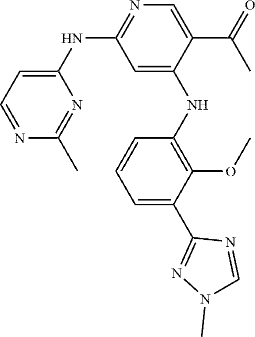 [SMILES: COc1c(Nc2cc(Nc3ccnc(C)n3)ncc2C(C)=O)cccc1-c1ncn(C)n1] | 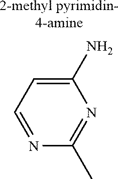 | ¹H NMR (400 MHz, Methanol-d4) δ 8.86 (s, 1H), 8.52 (s, 1H), 8.26 (m, 1H), 8.15 (m, 1H), 7.73 (m, 2H), 7.37 (m, 1H), 7.19 (m, 1H), 4.06 (s, 3H), 3.75 (s, 3H), 2.68 (s, 3H), 2.49 (s, 3H). | 431.15 |
| 7S | 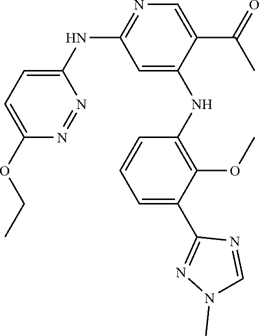 [SMILES: CCOc1ccc(Nc2cc(Nc3cccc(-c4ncn(C)n4)c3OC)c(C(C)=O)cn2)nn1] | 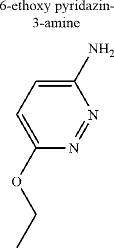 | ¹H NMR (400 MHz, DMSO-d6) δ 11.1 (br s, 1H), 10.9 (br s, 1H), 8.95 (s, 1H), 8.62 (s, 1H), 7.78 (m, 1H), 7.6 (m, 2H), 7.32 (m, 2H), 7.0 (m, 1H), 4.44 (m, 2H), 3.96 (s, 3H), 3.75 (s, 3H), 2.68 (s, 3H), 1.38 (m, 3H). | 461.10 |

| Cmpd. No. | Structure | Aniline | 1H NMR | MS (M+H)+ |
|---|---|---|---|---|
| 7T | 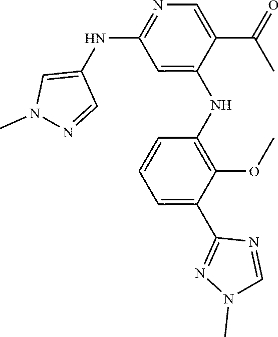 | 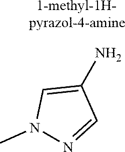 | ¹H NMR (400 MHz, Methanol-d4) δ 8.70 (s, 1H), 8.51 (s, 1H), 7.68 (s, 1H), 7.64 (m, 1H), 7.55 (m, 1H), 7.48 (s, 1H), 7.26 (m, 1H), 6.32 (s, 1H), 4.04 (s, 3H), 3.85 (s, 3H), 3.71 (s, 3H), 2.62 (s, 3H). | 419.15 |
| 7U | 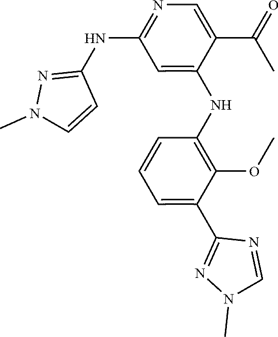 | 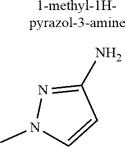 | ¹H NMR (300 MHz, Methanol-d4) δ 8.86 (s, 1H), 8.55 (s, 1H), 7.84 (m, 1H), 7.55 (m, 2H), 7.36 (m, 1H), 6.46 (s, 1H), 5.95 (s, 1H), 4.04 (s, 3H), 3.90 (s, 3H), 3.70 (s, 3H), 2.68 (s, 3H). | 419.10 |

### Example 8

Preparation of 6-(( 4-((2-methoxy-3-(1-methyl-1H-1,2,4-triazol-3-yl)phenyl)amino )-5-propionylpyridin-2-yl)amino)nicotinamide (Compound 8A)

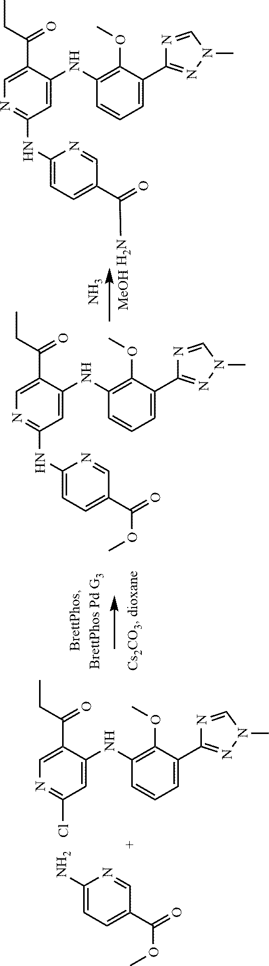

### Step 1: Methyl 6-((4-((2-methoxy-3-(1-methyl-1H-1,2,4-triazol-3-yl)phenyl)amino)-5-propionylpyridin-2-yl)amino)nicotinate

To a stirred solution of 1-(6-chloro-4-((2-methoxy-3-(1-methyl-1H-1,2,4-triazol-3-yl)phenyl)amino)pyridin-3-yl)propan-1-one (500 mg, 1.34 mmol) and methyl 6-aminopyridine-3-carboxylate (246 mg, 1.61 mol) in 1,4-dioxane (10 mL) was added cesium carbonate (1.31 g, 4.03 mmol), BrettPhos (144 mg, 0.37 mmol) and BrettPhos Pd G3 (122 mg, 0.14 mol) under a nitrogen atmosphere. The mixture was stirred for 3 hours at 90° C. The reaction mixture was filtered, and the filtrate was concentrated under vacuum. The residue was purified on a silica gel column eluting with methanol/dichloromethane (1:20) to yield methyl 6-((4-((2-methoxy-3-(1-methyl-1H-1,2,4-triazol-3-yl)phenyl)amino)-5-propionylpyridin-2-yl)amino)nicotinate (550 mg, 84%) as a solid.

### Step 2: 6-((4-((2-methoxy-3-(1-methyl-1H-1,2,4-triazol-3-yl)phenyl)amino)-5-propionylpyridin-2-yl)amino)nicotinamide

To a stirred solution of methyl 6-((4-((2-methoxy-3-(1-methyl-1H-1,2,4-triazol-3-yl)phenyl)amino)-5-propionylpyridin-2-yl)amino)nicotinate (100 mg, 0.2 mmol) in a 20 mL pressure reactor was added 7 M ammonia in methanol (4 mL). The vessel was sealed and heated to 90° C. for 16 hours. The solution was cooled, then concentrated under vacuum. The residue was purified on a silica gel column eluting with methanol/dichloromethane (1:20). The crude product was repurified by Prep-HPLC to yield 6-((4-((2-methoxy-3-(1-methyl-1H-1,2,4-triazol-3-yl)phenyl)amino)-5-propionylpyridin-2-yl)amino)nicotinamide (18 mg, 15%) as a solid.

$$\text{(ES,} m / z ) : {[ \text{M+H]}}^{+} 473.20$$

¹H NMR (300 MHz, Methanol-d₄) δ 8.99-8.88 (m, 2H), 8.54 (s, 1H), 8.30 (d, J = 8.7 Hz, 1H), 7.89 (d, J = 7.8, Hz, 1H), 7.60 (d, J = 7.9, Hz, 1H), 7.41 (t, J = 7.9 Hz, 1H), 7.08 (d, J= 8.7 Hz, 1H), 6.57 (s, 1H), 4.05 (s, 3H), 3.73 (s, 3H), 3.10-3.25 (m, 2H), 1.29 (t, J= 7.1 Hz, 3H).

### Preparation of Compounds 8B-8O

Compounds 8B-8O in TABLE 8 were prepared in a similar manner and according to the general synthetic schemes and procedures described herein.

TABLE 8

COMPOUNDS 8B THROUGH 8O

| Cmpd. No. | Structure | ¹H NMR | MS (M+H)⁺ |
|---|---|---|---|
| 8B | 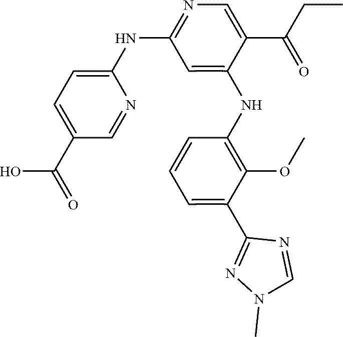 [SMILES: CCC(=O)c1cnc(Nc2ccc(C(=O)O)cn2)cc1Nc1cccc(-c2ncn(C)n2)c1OC] | 1H NMR (400 MHz, DMSO-d6) δ 11.09 (s, 1H), 10.36 (s, 1H), 8.89 (m, 1H), 8.68 (m, 1H), 8.56 (m, 1H), 8.11 (m, 1H), 7.96 (m, 1H), 7.72-7.64 (m, 3H), 7.33 (m, 1H), 3.96 (s, 3H), 3.75 (s, 3H), 3.12 (m, 2H), 1.14 (m, 3H). | 474.25 |
| 8C | 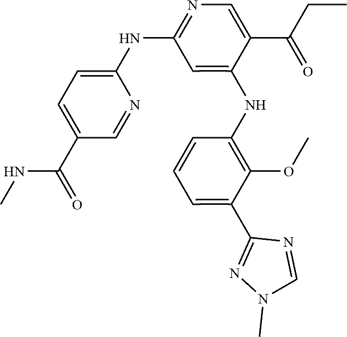 [SMILES: CCC(=O)c1cnc(Nc2ccc(C(=O)NC)cn2)cc1Nc1cccc(-c2ncn(C)n2)c1OC] | 1H NMR (300 MHz, Methanol-d4) δ 8.96 (m, 1H), 8.86 (m, 1H), 8.57 (m, 1H), 8.26 (m, 1H), 7.89 (m, 1H), 7.59 (m, 1H), 7.41 (m, 1H), 7.06 (m, 1H), 6.53 (m, 1H), 4.05 (s, 3H), 3.73 (s, 3H), 3.14 (m, 2H), 2.96 (s, 3H), 1.29 (m, 3H). | 487.15 |
| 8D | 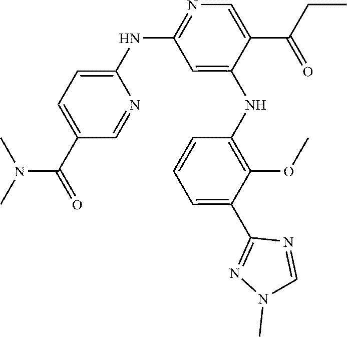 [SMILES: CCC(=O)c1cnc(Nc2ccc(C(=O)N(C)C)cn2)cc1Nc1cccc(-c2ncn(C)n2)c1OC] | 1H NMR (300 MHz, Methanol-d4) δ 8.85 (m, 1H), 8.51 (m, 1H), 8.34 (m, 1H), 7.82 (m, 1H), 7.76-7.68 (m, 3H), 7.46 (m, 1H), 7.36 (m, 1H), 4.05 (s, 3H), 3.74 (s, 3H), 3.11 (m, 8H), 1.27 (m, 3H). | 501.15 |
| 8E | 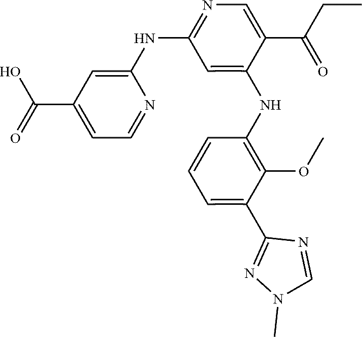 [SMILES: CCC(=O)c1cnc(Nc2cc(C(=O)O)ccn2)cc1Nc1cccc(-c2ncn(C)n2)c1OC] | 1H NMR (400 MHz, DMSO-d6) δ 11.11 (s, 1H), 10.14 (s, 1H), 8.89 (m, 1H), 8.57 (m, 1H), 8.29 (m, 1H), 8.11 (m, 1H), 7.96 (m, 1H), 7.71 (m, 1H), 7.64 (m, 1H), 7.33 (m, 2H), 3.96 (s, 3H), 3.75 (s, 3H), 3.10 (m, 2H), 1.14 (m, 3H). | 474.20 |
| 8F | 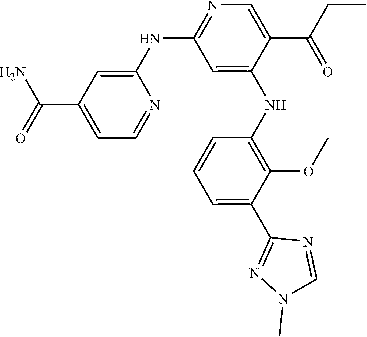 [SMILES: CCC(=O)c1cnc(Nc2cc(C(N)=O)ccn2)cc1Nc1cccc(-c2ncn(C)n2)c1OC] | 1H NMR (400 MHz, DMSO-d6) δ 11.10 (s, 1H), 10.10 (s, 1H), 8.89 (m, 1H), 8.57 (m, 1H), 8.30 (m, 1H), 8.10 (m, 1H), 8.02 (m, 1H), 7.88 (m, 1H), 7.73-7.62 (m, 3H), 7.35-7.25 (m, 2H), 3.96 (s, 3H), 3.76 (s, 3H), 3.12 (m, 2H), 1.15 (m, 3H). | 473.20 |
| 8G | 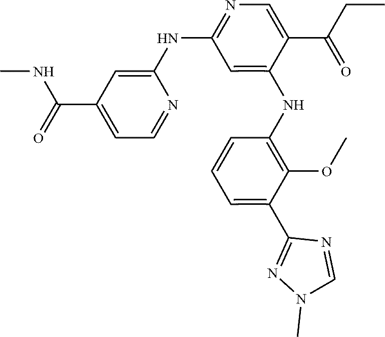 [SMILES: CCC(=O)c1cnc(Nc2cc(C(=O)NC)ccn2)cc1Nc1cccc(-c2ncn(C)n2)c1OC] | 1H NMR (400 MHz, DMSO-d6) δ 11.11 (s, 1H), 10.26 (s, 1H), 8.90 (m, 1H), 8.67-8.57 (m, 2H), 8.31 (m, 1H), 7.93 (m, 1H), 7.68 (m, 3H), 7.38-7.24 (m, 2H), 3.96 (s, 3H), 3.75 (s, 3H), 3.12 (m, 2H), 2.79 (m, 3H), 1.15 (m, 3H). | 487.30 |
| 8H | 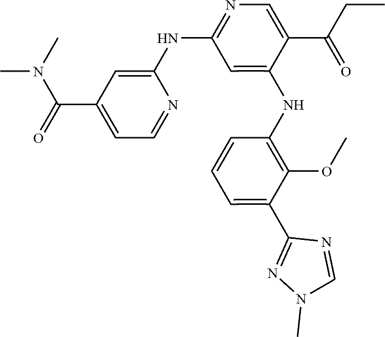 [SMILES: CCC(=O)c1cnc(Nc2cc(C(=O)N(C)C)ccn2)cc1Nc1cccc(-c2ncn(C)n2)c1OC] | 1H NMR (400 MHz, Methanol-d4) δ 8.84 (m, 1H), 8.50 (m, 1H), 8.28 (m, 1H), 7.76-7.66 (m, 3H), 7.58 (m, 1H), 7.34 (m, 1H), 6.90 (m, 1H), 4.04 (s, 3H), 3.74 (s, 3H), 3.13 (m, 5H), 3.02 (s, 3H), 1.26 (m, 3H). | 501.20 |
| 81 | 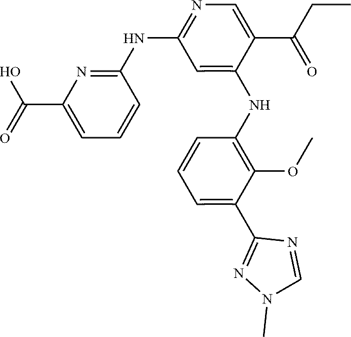 [SMILES: CCC(=O)c1cnc(Nc2cccc(C(=O)O)n2)cc1Nc1cccc(-c2ncn(C)n2)c1OC] | 1H NMR (300 MHz, Methanol-d4) δ 9.06 (m, 1H), 8.53 (m, 1H), 8.04 (m, 1H), 7.94-7.86 (m, 2H), 7.57 (m, 1H), 7.39 (m, 1H), 7.21 (m, 1H), 6.49 (m, 1H), 4.03 (s, 3H), 3.71 (s, 3H), 3.16 (m, 5H), 1.27 (m, 3H). | 474.15 |
| 8J | 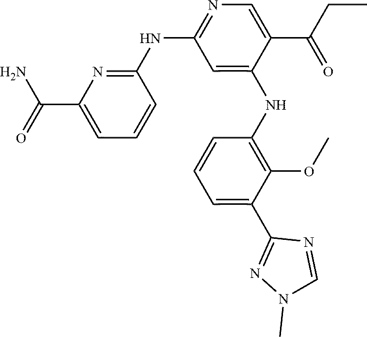 [SMILES: CCC(=O)c1cnc(Nc2cccc(C(N)=O)n2)cc1Nc1cccc(-c2ncn(C)n2)c1OC] | 1H NMR (300 MHz, DMSO-d6) δ 11.1 (s, 1H), 10.05 (s, 1H), 8.89 (m, 2H), 7.85 (m, 2H), 7.66 (m, 2H), 7.56 (m, 1H), 7.32 (m, 2H), 3.95 (s, 3H), 3.72 (s, 3H), 3.12 (m, 2H), 1.14 (m, 3H). | 473.15 |
| 8K | 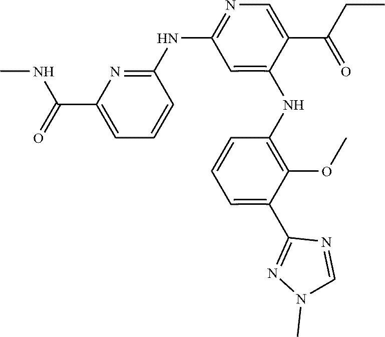 [SMILES: CCC(=O)c1cnc(Nc2cccc(C(=O)NC)n2)cc1Nc1cccc(-c2ncn(C)n2)c1OC] | 1H NMR (400 MHz, DMSO-d6) δ 11.16 (s, 1H), 11.0 (s, 1H), 8.96 (m, 1H), 8.59 (m, 1H), 7.98 (m, 1H), 7.78 (m, 1H), 7.68 (m, 1H), 7.63 (m, 1H), 7.41 (m, 1H), 7.31 (m, 1H), 6.92 (m, 1H), 3.96 (s, 3H), 3.75 (s, 3H), 3.15 (m, 2H), 2.81 (m, 3H), 1.16 (m, 3H). | 487.2 |
| 8L | 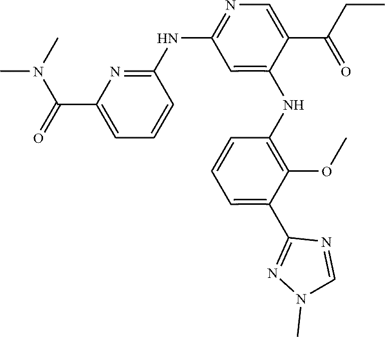 [SMILES: CCC(=O)c1cnc(Nc2cccc(C(=O)N(C)C)n2)cc1Nc1cccc(-c2ncn(C)n2)c1OC] | 1H NMR (400 MHz, DMSO-d6) δ 11.18 (s, 1H), 11.1 (s, 1H), 9.05 (m, 1H), 8.59 (m, 1H), 7.92 (m, 1H), 7.82 (m, 1H), 7.59 (m, 1H), 7.34 (m, 1H), 7.23 (m, 2H), 7.03 (m, 1H), 3.96 (s, 3H), 3.74 (s, 3H), 3.13 (m, 2H), 3.00 (s, 3H), 2.86 (s, 3H), 1.16 (m, 3H). | 501.20 |
| 8M | 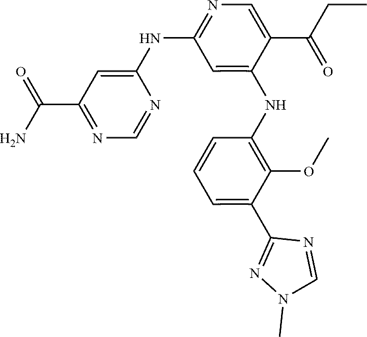 [SMILES: CCC(=O)c1cnc(Nc2cc(C(N)=O)ncn2)cc1Nc1cccc(-c2ncn(C)n2)c1OC] | 1H NMR (400 MHz, Methanol-d4) δ 8.97 (m, 1H), 8.93 (m, 1H), 8.53 (m, 1H), 7.87 (m, 1H), 7.64 (m, 1H), 7.57 (m, 1H), 7.38 (m, 1H), 6.70 (m, 1H), 4.03 (s, 3H), 3.70 (s, 3H), 3.15 (m, 2H), 1.27 (m, 3H). | 474.15 |
| 8N | 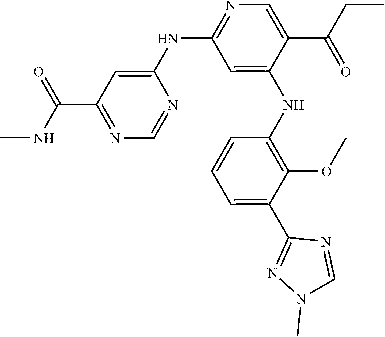 [SMILES: CCC(=O)c1cnc(Nc2cc(C(=O)NC)ncn2)cc1Nc1cccc(-c2ncn(C)n2)c1OC] | 1H NMR (400 MHz, Methanol-d4) δ 8.98 (m, 1H), 8.94 (m, 1H), 8.56 (m, 1H), 7.89 (m, 1H), 7.6 (m, 2H), 7.40 (m, 1H), 6.62 (m, 1H), 4.03 (s, 3H), 3.71 (s, 3H), 3.16 (m, 2H), 2.96 (s, 3H), 1.27 (m, 3H). | 488.15 |
| 8O | 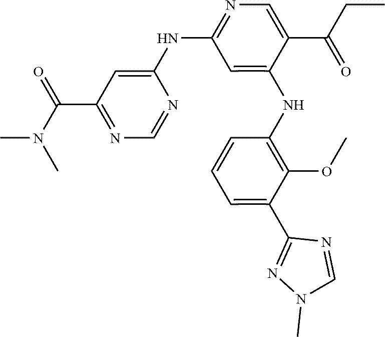 [SMILES: CCC(=O)c1cnc(Nc2cc(C(=O)N(C)C)ncn2)cc1Nc1cccc(-c2ncn(C)n2)c1OC] | 1H NMR (400 MHz, Methanol-d4) δ 8.95 (m, 2H), 8.55 (m, 1H), 7.89 (m, 1H), 7.60 (m, 1H), 7.41 (m, 1H), 7.19 (m, 1H), 6.69 (m, 1H), 4.05 (s, 3H), 3.73 (s, 3H), 3.22-3.05 (m, 8H), 1.28 (m, 3H). | 502.25 |

### Example 9

Preparation of 1-(6-((5-(( dimethylamino )methyl)pyridin-2-yl )amino )-4-( (2-methoxy-3-(1 -methyl-1H-1,2,4-triazol-3-yl)phenyl)amino )pyridin-3-yl)propan-1-one (Compound 9A)

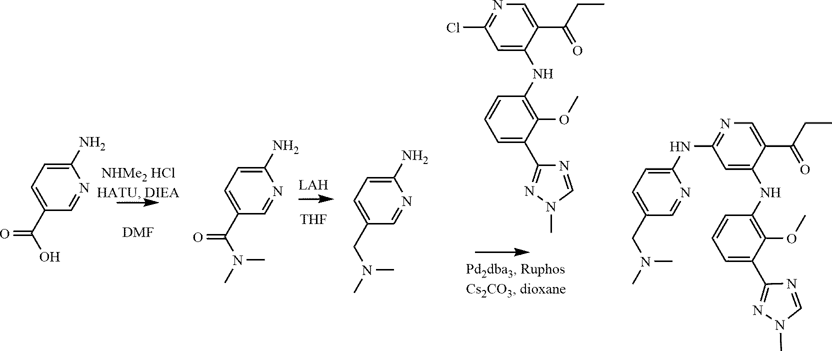

### Step 1: 6-amino-N,N-dimethylnicotinamide

A mixture of 6-aminonicotinic acid (1 g, 7.24 mmol), dimethylamine hydrochloride (709 mg, 8.69 mmol), 1-[bis(dimethylamino)methylene]-1H-1,2,3-triazolo[4,5-b]pyridinium 3-oxid hexafluorophosphate (3.03 g, 7.96 mmol) and diisopropylethylamine (2.81 g, 21.7 mmol) in N,N-dimethylformamide (10 mL) was stirred at 25° C. for 4 hours The reaction mixture was quenched with water (30 mL) and extracted with methanol in dichloromethane (10:90; 4 × 100 mL), the combined organic layer was dried over sodium sulfate and concentrated in vacuo. The residue was purified by column chromatography on silica gel eluting with methanol in dichloromethane (0 to 18%) to yield 6-amino-N,N-dimethylnicotinamide (600 mg, 50%) as a solid.

### Step 2: 5-((Dimethylamino )methyl)pyridin-2-amine

To a solution of lithium aluminum hydride (2.5 M in THF, 726 µL) was added 6-amino-N,N-dimethylnicotinamide (100 mg, 0.61 mmol) in tetrahydrofuran (3 mL) dropwise. The mixture was stirred at 45° C. for 16 hours. The reaction mixture was quenched with water (20 mL) and extracted with methanol in dichloromethane (10:90; 4 × 50 mL), dried over sodium sulfate and concentrated in vacuo to yield 5-(dimethylamino)methyl)pyridin-2-amine (40 mg, 43%) as a solid.

### Step 3: 1-(6-((5-((dimethylamino)methyl)pyridin-2-yl)amino)-4-((2-methoxy-3-(1-methyl-1H-1,2,4-triazol-3-yl)phenyl)amino)pyridin-3-yl)propan-1-one

A mixture of 1-(6-chloro-4-((2-methoxy-3-(1-methyl-1H-1,2,4-triazol-3-yl)phenyl)amino)pyridin-3-yl)propan-1-one (80 mg, 0.22 mmol), 5-((dimethylamino) methyl)pyridin-2-amine (36 mg, 0.24 mmol), tris(dibenzylideneacetone)dipalladium(0) (20 mg, 0.02 mmol), Ruphos (20 mg, 0.043 mmol) and cesium carbonate (210 mg, 0.65 mmol) in 1,4-dioxane (5 mL) was stirred at 100° C. for 2 hours under N2. The reaction mixture was cooled to room temperature, added water (15 mL) and extracted with ethyl acetate (3 × 40 mL), the combined organic layer was washed with brine (30 mL), dried over sodium sulfate and concentrated in vacuo. The residue was purified by prep-HPLC to yield 1-(6-((5-((dimethylamino)methyl)pyridin-2-yl)amino)-4-((2-methoxy-3-(1-methyl-1H-1,2,4-triazol-3-yl)phenyl)amino)pyridin-3-yl)propan-1-one (59 mg, 55%) as a solid.

$$\text{ESI-MS [M+H]+: 487} \text{.20} \text{.}$$

¹H NMR (400 MHz, DMSO-d6) δ 11.12 (s, 1H), 9.96 (s, 1H), 8.85 (s, 1H), 8.57 (s, 1H), 8.09-7.91 (m, 2H), 7.73-7.67 (m, 1H), 7.65-7.52 (m, 3H), 7.35-7.27 (m, 1H), 3.96 (s, 3H), 3.75 (s, 3H), 3.30 (s, 2H), 3.09 (q, J = 7.2 Hz, 2H), 2.12 (s, 6H), 1.13 (t, J = 7.2 Hz, 3H).

### Preparation of Compounds 9B-9UUUU

Compounds 9B-9UUUU in TABLE 9 were prepared in a similar manner and according to the general synthetic schemes and procedures described herein.

TABLE 9

COMPOUNDS 9B THROUGH 9UUUU

| Cmpd. No. | Structure | ¹H NMR | MS (M+H)⁺ |
|---|---|---|---|
| 9B | 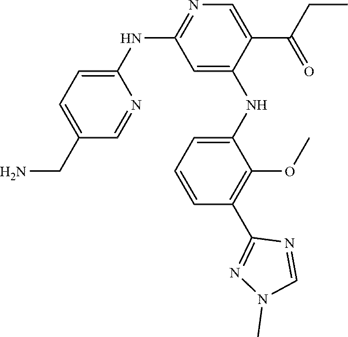 [SMILES: CCC(=O)c1cnc(Nc2ccc(CN)cn2)cc1Nc1cccc(-c2ncn(C)n2)c1OC] | 1H NMR (300 MHz, Methanol-d4) δ 8.96 (s, 1H), 8.56 (s, 1H), 8.51 (m, 1H), 7.97 (m, 1H), 7.89 (m, 1H), 7.61 (m, 1H), 7.41 (m, 1H), 7.09 (m, 1H), 6.53 (m, 1H), 4.20 (m, 2H), 4.05 (s, 3H), 3.73 (s, 3H), 3.16 (m, 2H), 1.29 (m, 3H). | 459.25 |
| 9C | 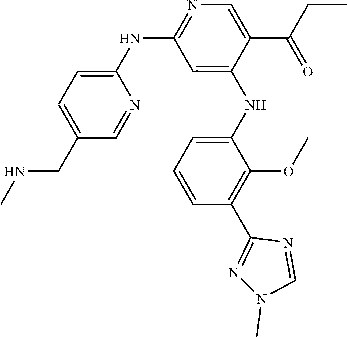 [SMILES: CCC(=O)c1cnc(Nc2ccc(CNC)cn2)cc1Nc1cccc(-c2ncn(C)n2)c1OC] | 1H NMR (300 MHz, Methanol-d4) δ 8.95 (s, 1H), 8.56 (s, 1H), 8.52 (m, 1H), 7.98 (m, 1H), 7.89 (m, 1H), 7.61 (m, 1H), 7.41 (m, 1H), 7.10 (m, 1H), 6.53 (m, 1H), 4.27 (m, 2H), 4.05 (s, 3H), 3.73 (s, 3H), 3.16 (m, 2H), 2.78 (m, 3H), 1.29 (m, 3H). | 473.20 |
| 9D | 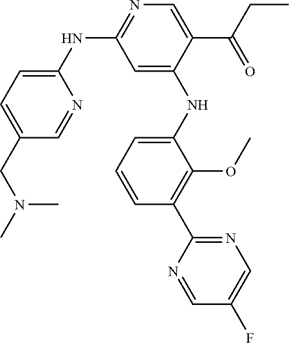 [SMILES: CCC(=O)c1cnc(Nc2ccc(CN(C)C)cn2)cc1Nc1cccc(-c2ncc(F)cn2)c1OC] | 1H NMR (400 MHz, Methanol-d4) δ 8.86 (m, 2H), 8.78 (m, 1H), 8.12 (m, 1H), 7.84 (m, 1H), 7.76 (m, 1H), 7.65-7.55 (m, 2H), 7.42-7.31 (m, 2H), 3.71 (s, 3H), 3.48 (m, 2H), 3.07 (m, 2H), 2.28 (s, 6H), 1.23 (m, 3H). | 502.30 |
| 9E | 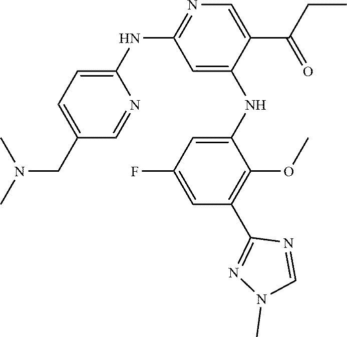 [SMILES: CCC(=O)c1cnc(Nc2ccc(CN(C)C)cn2)cc1Nc1cc(F)cc(-c2ncn(C)n2)c1OC] | 1H NMR (400 MHz, Methanol-d4) δ 8.81 (m, 1H), 8.50 (m, 1H), 8.18 (m, 2H), 7.65 (m, 2H), 7.36 (m, 2H), 4.04 (s, 3H), 3.78 (s, 3H), 3.46 (m, 2H), 3.07 (m, 2H), 2.27 (s, 6H), 1.26 (m, 3H). | 505.35 |
| 9F | 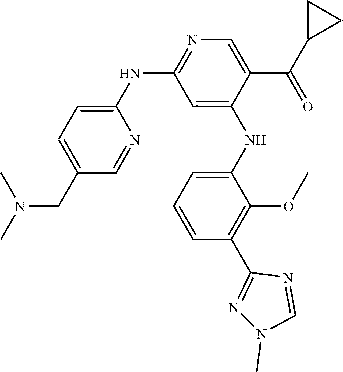 [SMILES: COc1c(Nc2cc(Nc3ccc(CN(C)C)cn3)ncc2C(=O)C2CC2)cccc1-c1ncn(C)n1] | 1H NMR (400 MHz, Methanol-d4) δ 8.95 (m, 1H), 8.46 (m, 1H), 8.10 (m, 1H), 7.84 (m, 1H), 7.72-7.60 (m, 3H), 7.40 (m, 1H), 7.31 (m, 1H), 4.00 (s, 3H), 3.69 (s, 3H), 3.43 (m, 2H), 2.77 (m, 1H), 2.24 (s, 6H), 1.25-1.10 (m, 4H). | 499.30 |
| 9G | 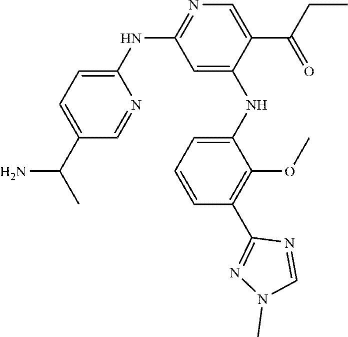 [SMILES: CCC(=O)c1cnc(Nc2ccc(C(C)N)cn2)cc1Nc1cccc(-c2ncn(C)n2)c1OC] | 1H NMR (400 MHz, Methanol-d4) δ 8.77 (m, 1H), 8.48 (m, 1H), 8.18 (m, 1H), 7.82 (m, 1H), 7.72-7.62 (m, 3H), 7.39 (m, 1H), 7.30 (m, 1H), 4.13 (m, 1H), 4.02 (s, 3H), 3.72 (s, 3H), 3.06 (m, 2H), 1.43 (m, 3H), 1.23 (m, 3H). | 473.20 |
| 9H | 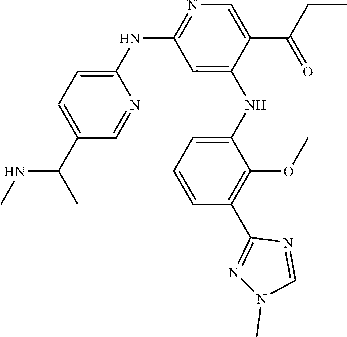 [SMILES: CCC(=O)c1cnc(Nc2ccc(C(C)NC)cn2)cc1Nc1cccc(-c2ncn(C)n2)c1OC] | 1H NMR (300 MHz, Methanol-d4) δ 8.80 (m, 1H), 8.57 (m, 1H), 8.15 (m, 1H), 7.85 (m, 1H), 7.76-7.65 (m, 3H), 7.44-7.31 (m, 2H), 4.04 (s, 3H), 3.7 (m, 4H), 3.07 (m, 2H), 2.27 (m, 1H), 1.41 (m, 3H), 1.26 (m, 3H). | 487.35 |
| 91 | 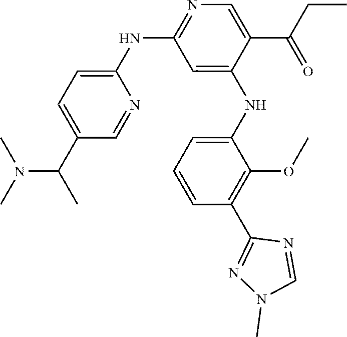 [SMILES: CCC(=O)c1cnc(Nc2ccc(C(C)N(C)C)cn2)cc1Nc1cccc(-c2ncn(C)n2)c1OC] | 1H NMR (300 MHz, Methanol-d4) δ 8.81 (m, 1H), 8.50 (m, 1H), 8.12 (m, 1H), 7.83 (m, 1H), 7.76-7.66 (m, 3H), 7.42 (m, 1H), 7.34 (m, 1H), 4.04 (s, 3H), 3.74 (s, 3H), 3.42 (m, 1H), 3.08 (m, 2H), 2.23 (m, 1H), 1.43 (m, 3H), 1.26 (m, 3H). | 501.35 |
| 9J | 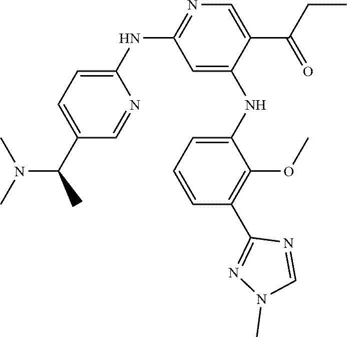 [SMILES: CCC(=O)c1cnc(Nc2ccc([C@@H](C)N(C)C)cn2)cc1Nc1cccc(-c2ncn(C)n2)c1OC] | 1H NMR (300 MHz, D₂O) δ 9.15 (m, 1H), 8.72 (m, 1H), 8.36 (m, 1H), 7.92 (m, 1H), 7.65 (m, 1H), 7.61 (m, 1H), 7.29 (m, 1H), 7.08 (m, 1H), 6.46 (m, 1H), 4.56 (m, 1H), 4.01 (s, 3H), 3.78 (s, 3H), 3.02 (m, 2H), 2.85-2.75 (m, 6H), 1.68 (m, 3H), 1.05 (m, 3H). | 501.25 |
| 9K | 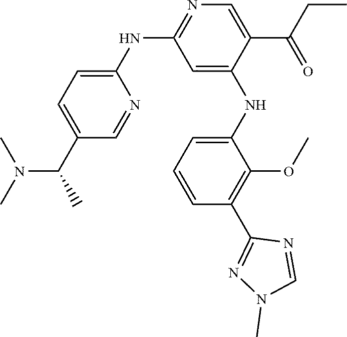 [SMILES: CCC(=O)c1cnc(Nc2ccc([C@H](C)N(C)C)cn2)cc1Nc1cccc(-c2ncn(C)n2)c1OC] | 1H NMR (300 MHz, D₂O) δ 9.08 (m, 1H), 8.73 (m, 1H), 8.37 (m, 1H), 7.94 (m, 1H), 7.67 (m, 1H), 7.55 (m, 1H), 7.28 (m, 1H), 7.06 (m, 1H), 6.44 (m, 1H), 4.56 (m, 1H), 4.03 (s, 3H), 3.53 (s, 3H), 3.02 (m, 2H), 2.78 (m, 3H), 2.65 (m, 3H), 1.67 (m, 3H), 1.03 (m, 3H). | 501.30 |
| 9L | 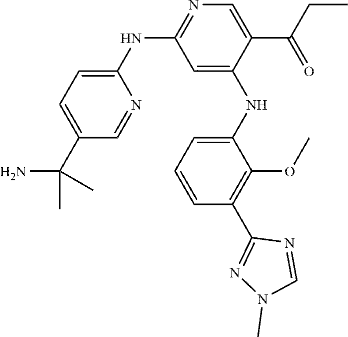 [SMILES: CCC(=O)c1cnc(Nc2ccc(C(C)(C)N)cn2)cc1Nc1cccc(-c2ncn(C)n2)c1OC] | 1H NMR (300 MHz, DMSO-d6) δ 11.09 (br s, 1H), 11.05 (br s, 1H), 8.95 (m, 1H), 8.57 (m, 1H), 8.39 (m, 1H), 8.02 (m, 1H), 7.83 (m, 1H), 7.59 (m, 1H), 7.35 (m, 1H), 7.08 (m, 1H), 6. 56 (m, 1H), 3.94 (s, 3H), 3.72 (s, 3H), 3.14 (m, 2H), 1.44 (s, 6H), 1.14 (m, 3H). | 488.10 |
| 9 M | 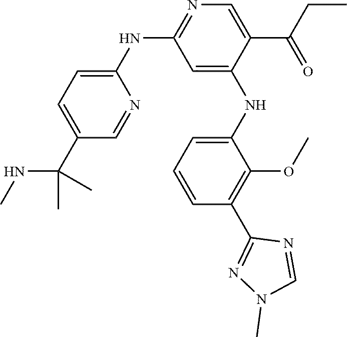 [SMILES: CCC(=O)c1cnc(Nc2ccc(C(C)(C)NC)cn2)cc1Nc1cccc(-c2ncn(C)n2)c1OC] | 1H NMR (300 MHz, DMSO-d6) δ 11.09 (m, 2H), 8.94 (m, 1H), 8.59 (m, 1H), 8.27 (m, 1H), 7.91 (m, 1H), 7.78 (m, 1H), 7.61 (m, 1H), 7.37 (m, 1H), 7.25 (m, 1H), 6. 9 (m, 1H), 3.96 (s, 3H), 3.75 (s, 3H), 3.15 (m, 2H), 3.02 (m, 3H), 1.48 (s, 6H), 1.17 (m, 3H). | 502.15 |
| 9N | 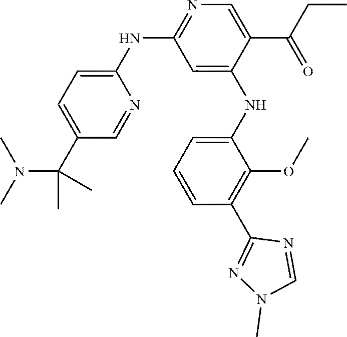 [SMILES: CCC(=O)c1cnc(Nc2ccc(C(C)(C)N(C)C)cn2)cc1Nc1cccc(-c2ncn(C)n2)c1OC] | 1H NMR (400 MHz, DMSO-d6) δ 11.12 (s, 1H), 9.92 (s, 1H), 8.85 (m, 1H), 8.57 (m, 1H), 8.21 (m, 1H), 7.94 (m, 1H), 7.8-7.55 (m, 4H), 7.31 (m, 1H), 3.96 (s, 3H), 3.74 (s, 3H), 3.10 (m, 2H), 2.09 (m, 6H), 1.29 (m, 6H), 1.14 (m, 3H). | 515.40 |
| 9O | 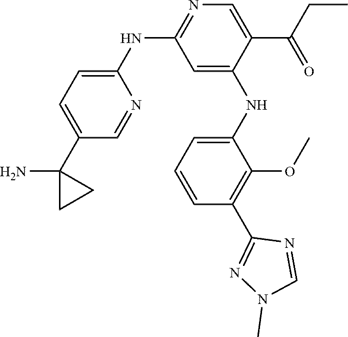 [SMILES: CCC(=O)c1cnc(Nc2ccc(C3(N)CC3)cn2)cc1Nc1cccc(-c2ncn(C)n2)c1OC] | 1H NMR (400 MHz, Methanol-d4) δ 8.85 (m, 1H), 8.54 (m, 2H), 7.99 (m, 1H), 7.87 (m, 1H), 7.67 (m, 1H), 7.41 (m, 1H), 7.05 (m, 1H), 6.48 (m, 1H), 4.06 (s, 3H), 3.70 (s, 3H), 3.12 (m, 2H), 1.44-1.25 (m, 7H). | 485.2 |
| 9P | 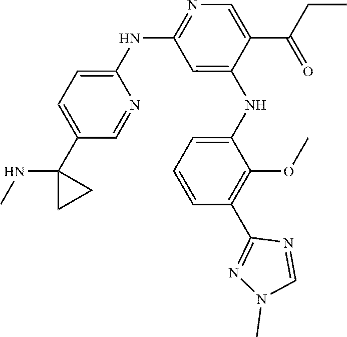 [SMILES: CCC(=O)c1cnc(Nc2ccc(C3(NC)CC3)cn2)cc1Nc1cccc(-c2ncn(C)n2)c1OC] | 1H NMR (400 MHz, Methanol-d4) δ 8.95 (m, 1H), 8.60 (m, 2H), 8.12 (m, 1H), 7.92 (m, 1H), 7.64 (m, 1H), 7.42 (m, 1H), 7.13 (m, 1H), 6.55 (m, 1H), 4.09 (s, 3H), 3.73 (s, 3H), 3.20 (m, 2H), 2.71 (m, 3H), 1.55-1.28 (m, 7H). | 499.25 |
| 9Q | 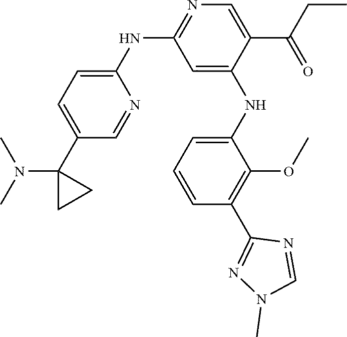 [SMILES: CCC(=O)c1cnc(Nc2ccc(C3(N(C)C)CC3)cn2)cc1Nc1cccc(-c2ncn(C)n2)c1OC] | 1H NMR (400 MHz, Methanol-d4) δ 8.95 (m, 1H), 8.61 (m, 2H), 8.12 (m, 1H), 7.86 (m, 1H), 7.60 (m, 1H), 7.39 (m, 1H), 7.10 (m, 1H), 6.57 (m, 1H), 4.04 (s, 3H), 3.72 (s, 3H), 3.15 (m, 2H), 2.89 (m, 6H), 1.62 (m, 2H), 1.37-1.25 (m, 5H). | 513.25 |
| 9R | 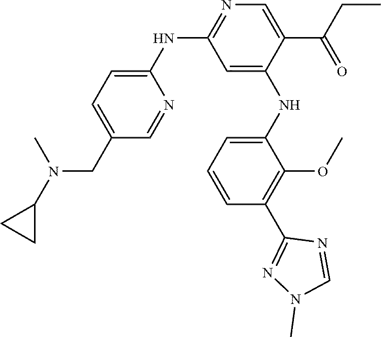 [SMILES: CCC(=O)c1cnc(Nc2ccc(CN(C)C3CC3)cn2)cc1Nc1cccc(-c2ncn(C)n2)c1OC] | 1H NMR (400 MHz, Methanol-d4) δ 8.98 (m, 1H), 8.83 (m, 1H), 8.55 (m, 1H), 8.04 (m, 1H), 7.87 (m, 1H), 7.64 (m, 1H), 7.39 (m, 1H), 7.13 (m, 1H), 6.65 (m, 1H), 4.56 (m, 2H), 4.09 (s, 3H), 3.73 (s, 3H), 3.15 (m, 2H), 3.00 (s, 3H), 2.92 (m, 1H), 1.24 (m, 3H), 0.97 (m, 4H). | 513.35 |
| 9S | 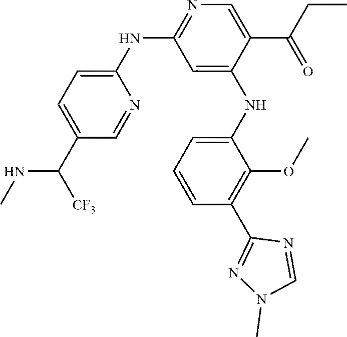 [SMILES: CCC(=O)c1cnc(Nc2ccc(C(NC)C(F)(F)F)cn2)cc1Nc1cccc(-c2ncn(C)n2)c1OC] | 1H NMR (400 MHz, Methanol-d4) δ 8.94 (s, 1H), 8.57 (m, 2H), 8.03 (m, 1H), 7.87 (m, 1H), 7.58 (m, 1H), 7.37 (m, 1H), 7.17 (m, 1H), 6.58 (m, 1H), 5.27 (m, 1H), 4.04 (s, 3H), 3.71 (s, 3H), 3.16 (m, 2H), 2.67 (m, 3H), 1.27 (m, 3H). | 541.20 |
| 9T | 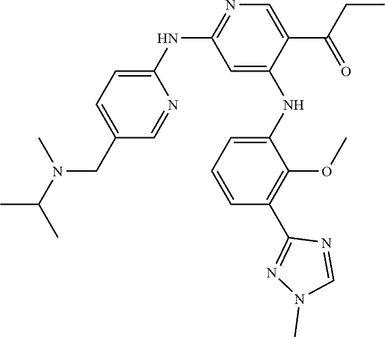 [SMILES: CCC(=O)c1cnc(Nc2ccc(CN(C)C(C)C)cn2)cc1Nc1cccc(-c2ncn(C)n2)c1OC] | 1H NMR (400 MHz, Methanol-d4) δ 8.80 (m, 1H), 8.50 (m, 1H), 8.13 (m, 1H), 7.85 (m, 1H), 7.74 (m, 1H), 7.66 (m, 1H), 7.41-7.31 (m, 2H), 4.04 (s, 3H), 3.75 (s, 3H), 3.56 (m, 2H), 3.10 (m, 2H), 2.94 (m, 1H), 2.19 (s, 3H), 1.29 (m, 3H), 1.26 (m, 6H). | 515.30 |
| 9U | 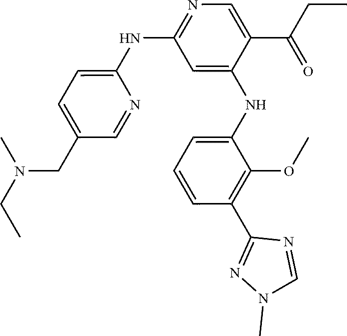 [SMILES: CCC(=O)c1cnc(Nc2ccc(CN(C)CC)cn2)cc1Nc1cccc(-c2ncn(C)n2)c1OC] | 1H NMR (400 MHz, Methanol-d4) δ 8.80 (m, 1H), 8.49 (m, 1H), 8.13 (m, 1H), 7.84 (m, 1H), 7.73 (m, 1H), 7.66 (m, 1H), 7.40 (m, 1H), 7.33 (m, 1H), 4.03 (s, 3H), 3.74 (s, 3H), 3.51 (m, 2H), 3.07 (m, 2H), 2.52 (m, 2H), 2.22 (s, 3H), 1.27 (m, 3H), 1.15 (m, 3H). | 501.35 |
| 9V | 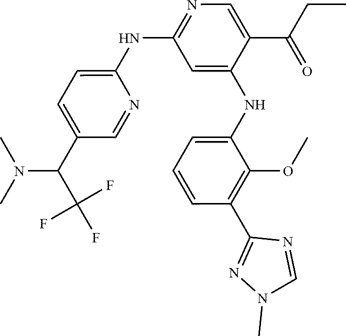 [SMILES: CCC(=O)c1cnc(Nc2ccc(C(N(C)C)C(F)(F)F)cn2)cc1Nc1cccc(-c2ncn(C)n2)c1OC] | 1H NMR (400 MHz, Methanol-d4) δ 8.93 (s, 1H), 8.58 (s, 1H), 8.47 (m, 1H), 7.97 (m, 1H), 7.86 (m, 1H), 7.58 (m, 1H), 7.38 (m, 1H), 7.10 (m, 1H), 6.54 (m, 1H), 4.65 (m, 1H), 4.04 (s, 3H), 3.72 (s, 3H), 3.16 (m, 2H), 2.51 (s, 6H), 1.27 (m, 3H). | 555.20 |
| 9W | 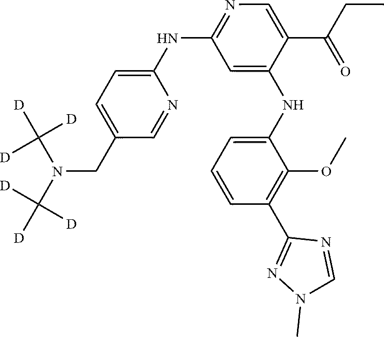 [SMILES: [2H]C([2H])([2H])N(Cc1ccc(Nc2cc(Nc3cccc(-c4ncn(C)n4)c3OC)c(C(=O)CC)cn2)nc1)C([2H])([2H])[2H]] | 1H NMR (400 MHz, Methanol-d4) δ 8.93 (m, 1H), 8.54 (m, 1H), 7.98 (m, 1H), 7.87 (m, 1H), 7.58 (m, 1H), 7.39 (m, 1H), 7.10 (m, 1H), 6.51 (m, 1H), 4.37 (s, 2H), 4.03 (s, 3H), 3.71 (s, 3H), 3.13 (m, 2H), 1.27 (m, 3H). | 493.30 |
| 9X | 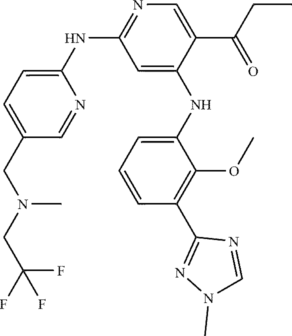 [SMILES: CCC(=O)c1cnc(Nc2ccc(CN(C)CC(F)(F)F)cn2)cc1Nc1cccc(-c2ncn(C)n2)c1OC] | 1H NMR (400 MHz, Methanol-d4) δ 8.94 (s, 1H), 8.55 (s, 1H), 8.35 (m, 1H), 7.92-7.86 (m, 2H), 7.60 (m, 1H), 7.40 (m, 1H), 7.03 (m, 1H), 6.49 (m, 1H), 4.05 (s, 3H), 3.78 (s, 2H), 3.73 (s, 3H), 3.21-3.13 (m, 4H), 2.45 (m, 3H), 1.28 (m, 3H). | 555.35 |
| 9Y | 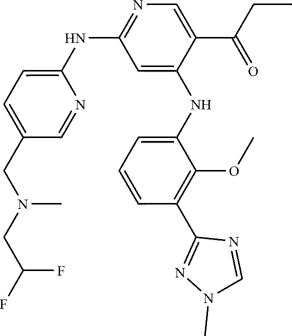 [SMILES: CCC(=O)c1cnc(Nc2ccc(CN(C)CC(F)F)cn2)cc1Nc1cccc(-c2ncn(C)n2)c1OC] | 1H NMR (400 MHz, Methanol-d4) δ 8.97 (s, 1H), 8.61 (s, 1H), 8.53 (m, 1H), 8.02 (m, 1H), 7.89 (m, 1H), 7.62 (m, 1H), 7.40 (m, 1H), 7.12 (m, 1H), 6.6-6.3 (m, 2H), 4.51 (m, 2H), 4.06 (s, 3H), 3.74-3.63 (m, 5H), 3.18 (m, 2H), 2.94 (m, 3H), 1.29 (m, 3H). | 537.20 |
| 9Z | 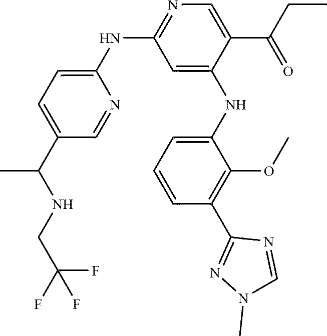 [SMILES: CCC(=O)c1cnc(Nc2ccc(C(C)NCC(F)(F)F)cn2)cc1Nc1cccc(-c2ncn(C)n2)c1OC] | 1H NMR (400 MHz, Methanol-d4) δ 8.80 (m, 1H), 8.50 (m, 1H), 8.14 (m, 1H), 7.81 (m, 1H), 7.75-7.65 (m, 3H), 7.41 (m, 1H), 7.33 (m, 1H), 4.04 (s, 3H), 3.89 (m, 1H), 3.74 (s, 3H), 3.14-3.0 (m, 4H), 1.38 (m, 3H), 1.24 (m, 3H). | 555.35 |
| 9AA | 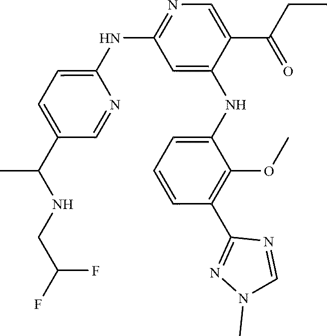 [SMILES: CCC(=O)c1cnc(Nc2ccc(C(C)NCC(F)F)cn2)cc1Nc1cccc(-c2ncn(C)n2)c1OC] | 1H NMR (400 MHz, Methanol-d4) δ 8.80 (m, 1H), 8.50 (m, 1H), 8.14 (m, 1H), 7.82 (m, 1H), 7.75-7.65 (m, 3H), 7.41 (m, 1H), 7.34 (m, 1H), 5.87 (m, 1H), 4.04 (s, 3H), 3.84 (m, 1H), 3.74 (s, 3H), 3.09 (m, 2H), 2.9-2.65 (m, 2H), 1.38 (m, 3H), 1.26 (m, 3H). | 537.35 |
| 9BB | 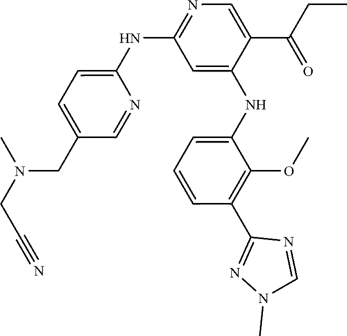 [SMILES: CCC(=O)c1cnc(Nc2ccc(CN(C)CC#N)cn2)cc1Nc1cccc(-c2ncn(C)n2)c1OC] | 1H NMR (400 MHz, Methanol-d4) δ 8.77 (m, 1H), 8.47 (m, 1H), 8.11 (m, 1H), 7.82 (m, 1H), 7.72-7.61 (m, 3H), 7.39 (m, 1H), 7.30 (m, 1H), 4.02 (s, 3H), 3.72 (s, 3H), 3.6-3.54 (m, 4H), 3.06 (m, 2H), 2.37 (m, 3H), 1.25 (m, 3H). | 512.20 |
| 9CC | 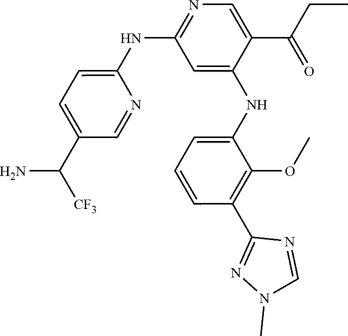 [SMILES: CCC(=O)c1cnc(Nc2ccc(C(N)C(F)(F)F)cn2)cc1Nc1cccc(-c2ncn(C)n2)c1OC] | 1H NMR (400 MHz, Methanol-d4) δ 8.93 (s, 1H), 8.57 (m, 1H), 8.54 (m, 1H), 8.03 (m, 1H), 7.87 (m, 1H), 7.57 (m, 1H), 7.37 (m, 1H), 7.13 (m, 1H), 6.55 (m, 1H), 5.39 (m, 1H), 4.03 (s, 3H), 3.71 (s, 3H), 3.16 (m, 2H), 1.27 (m, 3H). | 527.15 |
| 9DD | 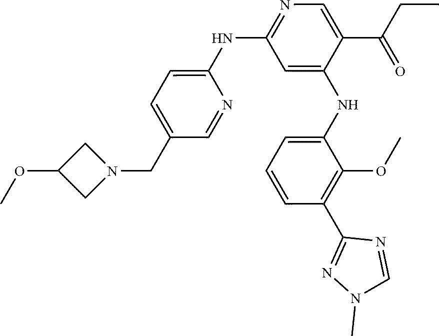 [SMILES: CCC(=O)c1cnc(Nc2ccc(CN3CC(OC)C3)cn2)cc1Nc1cccc(-c2ncn(C)n2)c1OC] | 1H NMR (400 MHz, Methanol-d4) δ 8.80 (m, 1H), 8.50 (m, 1H), 8.12 (m, 1H), 7.86 (m, 1H), 7.73 (m, 1H), 7.68-7.60 (m, 2H), 7.40-7.32 (m, 2H), 4.05 (m, 4H), 3.74 (s, 3H), 3.61-3.55 (m, 4H), 3.27 (s, 3H), 3.07 (m, 4H), 1.26 (m, 3H). | 529.35 |
| 9EE | 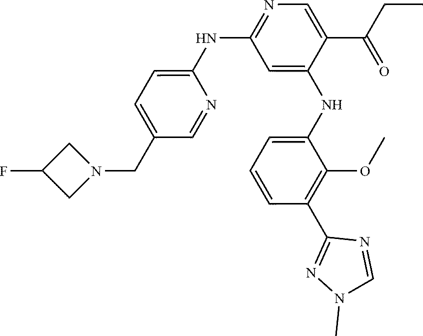 [SMILES: CCC(=O)c1cnc(Nc2ccc(CN3CC(F)C3)cn2)cc1Nc1cccc(-c2ncn(C)n2)c1OC] | 1H NMR (400 MHz, Methanol-d4) δ 8.80 (m, 1H), 8.50 (m, 1H), 8.12 (m, 1H), 7.86 (m, 1H), 7.75-7.61 (m, 3H), 7.40-7.32 (m, 2H), 5.23-5.06 (m, 1H), 4.04 (s, 3H), 3.74 (s, 3H), 3.66-3.58 (m, 4H), 3.29 (m, 2H), 3.10 (m, 2H), 1.26 (m, 3H). | 517.30 |
| 9FF | 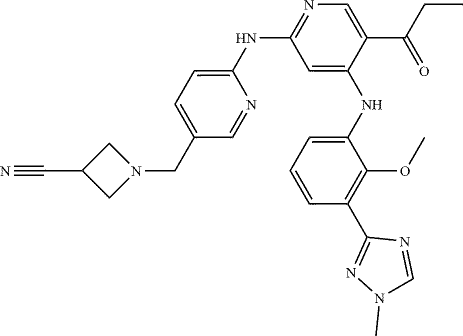 [SMILES: CCC(=O)c1cnc(Nc2ccc(CN3CC(C#N)C3)cn2)cc1Nc1cccc(-c2ncn(C)n2)c1OC] | 1H NMR (400 MHz, Methanol-d4) δ 8.80 (m, 1H), 8.51 (m, 1H), 8.12 (m, 1H), 7.85 (m, 1H), 7.75-7.61 (m, 3H), 7.40-7.33 (m, 2H), 4.05 (s, 3H), 3.74 (s, 3H), 3.59-3.53 (m, 4H), 3.46-3.35 (m, 3H), 3.10 (m, 2H), 1.27 (m, 3H). | 524.35 |
| 9GG | 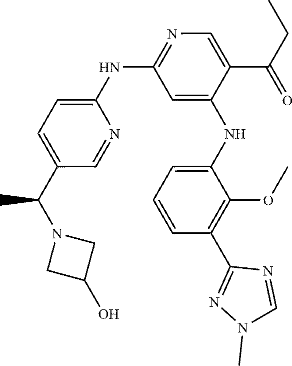 [SMILES: CCC(=O)c1cnc(Nc2ccc([C@H](C)N3CC(O)C3)cn2)cc1Nc1cccc(-c2ncn(C)n2)c1OC] | 1H NMR (400 MHz, Methanol-d4) δ 8.80 (s, 1H), 8.50 (s, 1H), 8.13 (d, J = 2.3 Hz, 1H), 7.86 (s, 1H), 7.75 -7.62 (m, 3H), 7.42 - 7.30 (m, 2H), 4.35 - 4.31 (m, 1H), 4.04 (s, 3H), 3.76 - 3.73 (m, 4H), 3.40 - 3.35 (m, 1H), 3.30 (m, 1H), 3.09 (q, J = 7.3 Hz, 2H), 2.95 - 2.84 (m, 2H), 1.29 -1.23 (m, 6H). | 529.2 |
| 9HH | 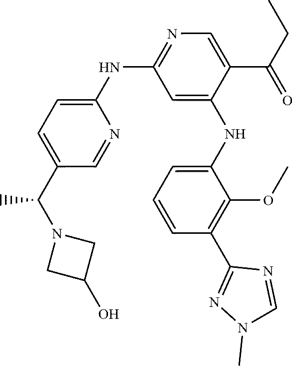 [SMILES: CCC(=O)c1cnc(Nc2ccc([C@@H](C)N3CC(O)C3)cn2)cc1Nc1cccc(-c2ncn(C)n2)c1OC] | 1H NMR (400 MHz, Methanol-d4) δ 8.80 (s, 1H), 8.50 (s, 1H), 8.13 (d, J = 2.3 Hz, 1H), 7.84 (s, 1H), 7.79 -7.74 (m, 1H), 7.79 - 7.66 (m, 2H), 7.39 (d, J = 8.6 Hz, 1H), 7.34 (t, J = 7.9 Hz, 1H), 4.39 - 4.25 (m, 1H), 4.04 (s, 3H), 3.78 (t, J = 7.3 Hz, 1H), 3.74 (s, 3H), 3.40 - 3.48 (m, 2H), 3.15 - 3.05 (m, 2H), 2.98 (t, J = 7.1 Hz, 1H), 2.88 (t, J = 7.1 Hz, 1H), 1.39 - 1.23 (m, 6H). | 529.3 |
| 9II | 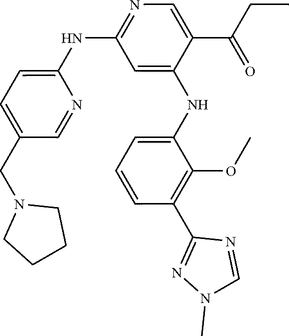 [SMILES: CCC(=O)c1cnc(Nc2ccc(CN3CCCC3)cn2)cc1Nc1cccc(-c2ncn(C)n2)c1OC] | 1H NMR (300 MHz, Methanol-d4) δ 8.97 (s, 1H), 8.60 (s, 1H), 8.55 (m, 1H), 8.02 (m, 1H), 7.89 (m, 1H), 7.62 (m, 1H), 7.41 (m, 1H), 7.11 (m, 1H), 6.58 (s, 1H), 4.46 (s, 2H), 4.06 (s, 3H), 3.74 (s, 3H), 3. 58 (m, 2H), 3.34-3.15 (m, 4H), 2.20 (m, 2H), 2.06 (m, 2H), 1.29 (m, 3H). | 513.2 |
| 9JJ | 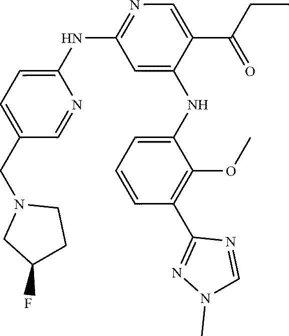 [SMILES: CCC(=O)c1cnc(Nc2ccc(CN3CC[C@@H](F)C3)cn2)cc1Nc1cccc(-c2ncn(C)n2)c1OC] | 1H NMR (400 MHz, Methanol-d4) δ 8.78 (s, 1H), 8.48 (s, 1H), 8.13 (m, 1H), 7.84 (m, 1H), 7.73 (m, 1H), 7.67 (m, 2H), 7.38 (m, 1H), 7.32 (m, 1H), 5.2 (m, 1H), 4.03 (s, 3H), 3.74 (s, 3H), 3.62 (m, 2H), 3.08 (m, 2H), 2.86 (m, 2H), 2.69 (m, 1H), 2.45 (m, 1H), 2.22 (m, 1H), 2.04 (m, 1H), 1.23 (m, 3H). | 531.25 |
| 9KK | 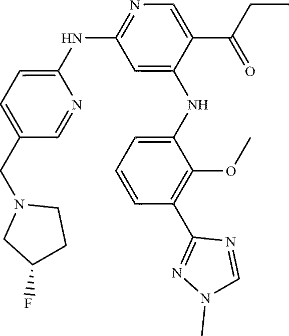 [SMILES: CCC(=O)c1cnc(Nc2ccc(CN3CC[C@H](F)C3)cn2)cc1Nc1cccc(-c2ncn(C)n2)c1OC] | 1H NMR (400 MHz, Methanol-d4) δ 8.79 (s, 1H), 8.49 (s, 1H), 8.15 (m, 1H), 7.85 (m, 1H), 7.73 (m, 1H), 7.67 (m, 2H), 7.38 (m, 1H), 7.34 (m, 1H), 5.18 (m, 1H), 4.03 (s, 3H), 3.74 (s, 3H), 3.62 (m, 2H), 3.08 (m, 2H), 2.86 (m, 2H), 2.69 (m, 1H), 2.45 (m, 1H), 2.22 (m, 1H), 2.04 (m, 1H), 1.25 (m, 3H). | 531.35 |
| 9LL | 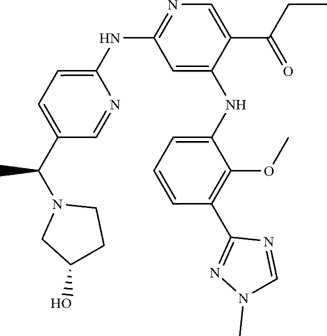 [SMILES: CCC(=O)c1cnc(Nc2ccc([C@H](C)N3CC[C@H](O)C3)cn2)cc1Nc1cccc(-c2ncn(C)n2)c1OC] | 1H NMR (400 MHz, Methanol-d4) δ 8.80 (s, 1H), 8.50 (s, 1H), 8.15 (d, J = 2.3 Hz, 1H), 7.84 (s, 1H), 7.78 -7.69 (m, 2H), 7.67 (dd, J = 7.8, 1.6 Hz, 1H), 7.40 (d, J = 8.6 Hz, 1H), 7.34 (t, J = 7.9 Hz, 1H), 4.38 - 4.28 (m, 1H), 4.04 (s, 3H), 3.74 (s, 3H), 3.42 - 3.34 (m, 1H), 3.08 (q, J = 7.3 Hz, 2H), 2.75 - 2.52 (m, 4H), 2.20 -2.10 (m, 1H), 1.68 - 1.58 (m, 1H), 1.42 (d, J = 6.6 Hz, 3H), 1.26 (t, J = 7.3 Hz, 3H). | 543.3 |
| 9MM | 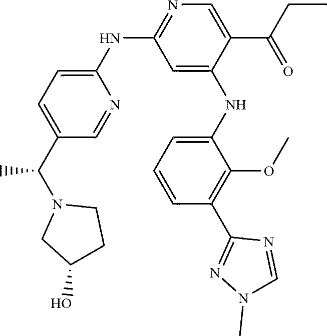 [SMILES: CCC(=O)c1cnc(Nc2ccc([C@@H](C)N3CC[C@H](O)C3)cn2)cc1Nc1cccc(-c2ncn(C)n2)c1OC] | 1H NMR (400 MHz, Methanol-d4) δ 8.80 (s, 1H), 8.50 (s, 1H), 8.14 (d, J = 2.3 Hz, 1H), 7.84 (s, 1H), 7.78 -7.63 (m, 3H), 7.40 (d, J = 8.6 Hz, 1H), 7.34 (t, J = 7.9 Hz, 1H), 4.38 -4.30 (m, 1H), 4.04 (s, 3H), 3.74 (s, 3H), 3.31 (s, 1H) 3.08 (q, J = 7.3 Hz, 2H), 2.85 (dd, J = 10.5, 6.2 Hz, 2H), 2.44 - 2.33 (m, 2H), 2.15 - 2.07 (m, 1H), 1.75 - 1.65 (m, 1H), 1.42 (d, J = 6.6 Hz, 3H), 1.26 (t, J = 7.3 Hz, 3H). | 543.3 |
| 9NN | 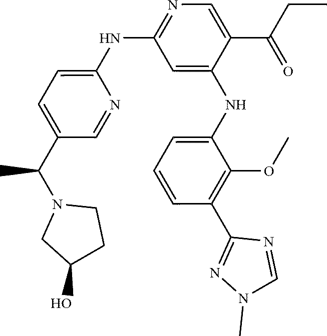 [SMILES: CCC(=O)c1cnc(Nc2ccc([C@H](C)N3CC[C@@H](O)C3)cn2)cc1Nc1cccc(-c2ncn(C)n2)c1OC] | 1H NMR (400 MHz, Methanol-d4) δ 8.77 (s, 1H), 8.49 (s, 1H), 8.13 (d, J = 2.4 Hz, 1H), 7.83 (s, 1H), 7.76 -7.62 (m, 3H), 7.59- 7.39 (m, 2H), 4.35 - 4.25 (m, 1H), 4.03 (s, 3H), 3.74 (s, 3H), 3.39 - 3.28 (m, 1H), 3.06 (q, J = 7.3 Hz, 2H), 2.72 - 2.49 (m, 4H), 2.18 - 2.08 (m, 1H), 1.75 -1.68 (m, 1H), 1.40 (d, J = 6.6 Hz, 3H), 1.23 (t, J = 7.2 Hz, 3H). | 543.3 |
| 9OO | 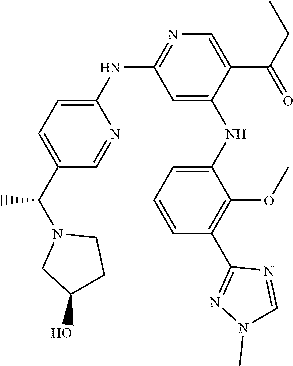 [SMILES: CCC(=O)c1cnc(Nc2ccc([C@@H](C)N3CC[C@@H](O)C3)cn2)cc1Nc1cccc(-c2ncn(C)n2)c1OC] | 1H NMR (400 MHz, Methanol-d4) δ 8.80 (s, 1H), 8.50 (s, 1H), 8.14 (d, J = 2.4 Hz, 1H), 7.85 (s, 1H), 7.78 -7.63 (m, 3H), 7.40 (d, J = 8.6 Hz, 1H), 7.34 (t, J = 7.9 Hz, 1H), 4.38 -4.33 (m, 1H), 4.04 (s, 3H), 3.74 (s, 3H), 3.30 (s, 1H), 3.08 (q, J = 7.3 Hz, 2H), 2.85 - 2.80 (m, 2H), 2.42 -2.37 (m, 2H), 2.17 - 2.08 (m, 1H), 1.79 - 1.67 (m, 1H), 1.42 (d, J = 6.6 Hz, 3H), 1.26 (t, J = 7.3 Hz, 3H). | 543.3 |
| 9PP | 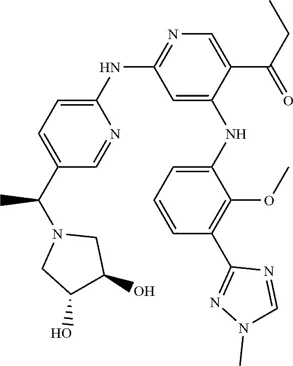 [SMILES: CCC(=O)c1cnc(Nc2ccc([C@H](C)N3C[C@@H](O)[C@H](O)C3)cn2)cc1Nc1cccc(-c2ncn(C)n2)c1OC] | 1H NMR (400 MHz, Methanol-d4) δ 8.80 (s, 1H), 8.50 (s, 1H), 8.15 (d, J = 2.3 Hz, 1H), 7.84 (s, 1H), 7.78 -7.60 (m, 2H), 7.66 (dd, J = 7.8, 1.6 Hz, 1H), 7.45 - 7.27 (m, 2H), 4.04 (m, 5H), 3.74 (s, 3H), 3.44 - 3.36 (m, 1H), 3.13 - 2.96 (m, 4H), 2.40 (dd, J = 10.3, 3.6 Hz, 2H), 1.41 (d, J = 6.6 Hz, 3H), 1.26 (t, J = 7.3 Hz, 3H). | 559.3 |
| 9QQ | 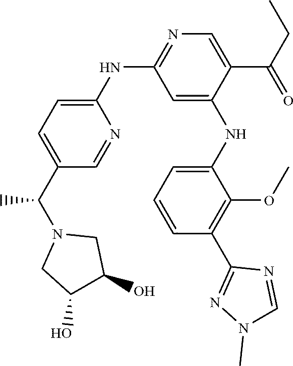 [SMILES: CCC(=O)c1cnc(Nc2ccc([C@@H](C)N3C[C@@H](O)[C@H](O)C3)cn2)cc1Nc1cccc(-c2ncn(C)n2)c1OC] | 1H NMR (400 MHz, Methanol-d4) δ 8.80 (s, 1H), 8.50 (s, 1H), 8.14 (d, J = 2.3 Hz, 1H), 7.83 (s, 1H), 7.79 -7.70 (m, 2H), 7.66 (dd, J = 7.9, 1.6 Hz, 1H), 7.39 (d, J = 8.6 Hz, 1H), 7.35 (d, J = 7.9 Hz, 1H), 4.14 - 3.99 (m, 5H), 3.74 (s, 3H), 3.44 - 3.36 (m, 1H), 3.08 (q, J = 7.3 Hz, 2H), 2.85 (dd, J = 10.2, 5.7 Hz, 2H), 2.57 (dd, J = 10.4, 3.6 Hz, 2H), 1.39 (d, J = 6.6 Hz, 3H), 1.26 (t, J = 7.3 Hz, 3H). | 559.3 |
| 9RR | 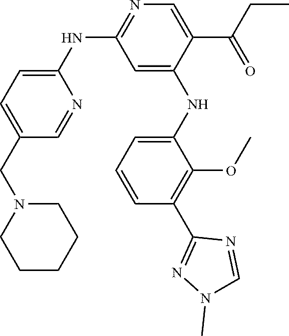 [SMILES: CCC(=O)c1cnc(Nc2ccc(CN3CCCCC3)cn2)cc1Nc1cccc(-c2ncn(C)n2)c1OC] | 1H NMR (300 MHz, Methanol-d4) δ 8.78 (s, 1H), 8.48 (s, 1H), 8.10 (m, 1H), 7.84 (m, 1H), 7.72 (m, 1H), 7.64 (m, 2H), 7.38 (m, 1H), 7.31 (m, 1H), 4.01 (s, 3H), 3.72 (s, 3H), 3.44 (s, 2H), 3.05 (m, 2H), 2.4 (m, 4H), 1.60 (m, 4H), 1.45 (m, 2H), 1.23 (m, 3H). | 527.4 |
| 9SS | 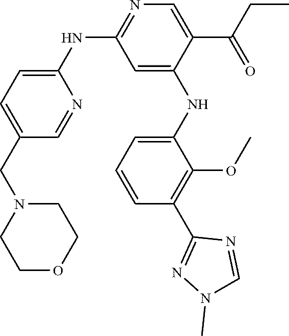 [SMILES: CCC(=O)c1cnc(Nc2ccc(CN3CCOCC3)cn2)cc1Nc1cccc(-c2ncn(C)n2)c1OC] | 1H NMR (300 MHz, Methanol-d4) δ 8.96 (m, 1H), 8.64-8.54 (m, 2H), 8.02 (m, 1H), 7.89 (m, 1H), 7.60 (m, 1H), 7.42 (m, 1H), 7.11 (m, 1H), 6.53 (m, 1H), 4.44 (s, 2H), 4.06-3.74 (m, 10H) 3.4-3.2 (m, 4H), 3.16 (m, 2H), 1.29 (m, 3H). | 529.25 |
| 9TT | 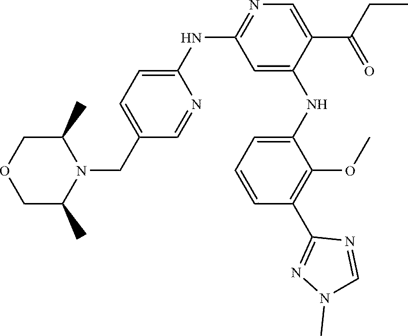 [SMILES: CCC(=O)c1cnc(Nc2ccc(CN3[C@H](C)COC[C@@H]3C)cn2)cc1Nc1cccc(-c2ncn(C)n2)c1OC] | 1H NMR (300 MHz, Methanol-d4) δ 8.81 (s, 1H), 8.50 (s, 1H), 8.22 (m, 1H), 7.74-7.6 (m, 4H), 7.37-7.29 (m, 2H), 4.04 (s, 3H), 3.87 (m, 2H), 3.74-3.68 (m, 5H), 3.25 (m, 2H), 3.11 (m, 2H), 2.63 (m, 2H), 1.31 (m, 3H), 1.02 (m, 6H). | 557.40 |
| 9UU | 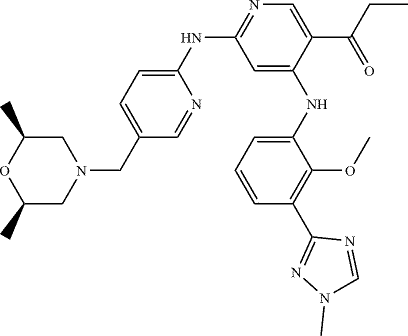 [SMILES: CCC(=O)c1cnc(Nc2ccc(CN3C[C@@H](C)O[C@@H](C)C3)cn2)cc1Nc1cccc(-c2ncn(C)n2)c1OC] | 1H NMR (300 MHz, Methanol-d4) δ 8.96 (s, 1H), 8.54 (m, 2H), 8.01 (m, 1H), 7.90 (m, 1H), 7.59 (m, 1H), 7.41 (m, 1H), 7.11 (m, 1H), 6.53 (m, 1H), 4.41 (m, 1H), 4.05 (s, 3H), 3.87 (m, 2H), 3.73 (s, 3H), 3.42 (m, 2H), 3.16 (m, 2H), 2.86 (m, 2H), 1.31-1.24 (m, 9H). | 557.30 |
| 9VV | 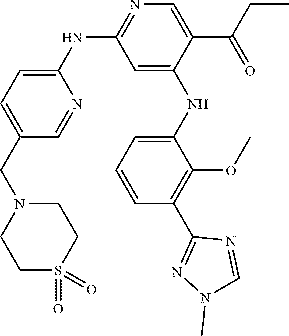 [SMILES: CCC(=O)c1cnc(Nc2ccc(CN3CCS(=O)(=O)CC3)cn2)cc1Nc1cccc(-c2ncn(C)n2)c1OC] | 1H NMR (300 MHz, Methanol-d4) δ 8.80 (s, 1H), 8.50 (s, 1H), 8.14 (m, 1H), 7.83 (m, 1H), 7.75-7.65 (m, 3H), 7.42-7.31 (m, 2H), 4.04 (s, 3H), 3.74 (s, 3H), 3.65 (m, 2H), 3.12-2.97 (m, 10H), 1.27 (m, 3H). | 577.25 |
| 9WW | 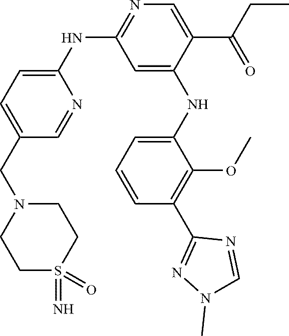 [SMILES: CCC(=O)c1cnc(Nc2ccc(CN3CCS(=N)(=O)CC3)cn2)cc1Nc1cccc(-c2ncn(C)n2)c1OC] | 1H NMR (300 MHz, DMSO-d6) δ 11.11 (br s, 1H), 9.99 (br s, 1H), 8.85 (m, 1H), 8.57 (m, 1H), 8.11 (m, 1H), 7.98 (m, 1H), 7.71-7.53 (m, 4H), 7.33 (m, 1H), 3.96 (s, 3H), 3.75 (s, 3H), 3.58 (m, 2H), 3.1 (m, 2H), 2.99 (m, 4H), 2.8 (m, 4H), 1.13 (m, 3H). | 576.25 |
| 9XX | 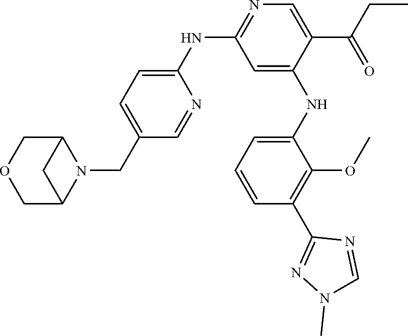 [SMILES: CCC(=O)c1cnc(Nc2ccc(CN3C4COCC3C4)cn2)cc1Nc1cccc(-c2ncn(C)n2)c1OC] | 1H NMR (400 MHz, Methanol-d4) δ 8.79 (m, 1H), 8.50 (m, 1H), 8.20 (m, 1H), 7.84 (m, 1H), 7.75-7.66 (m, 3H), 7.40-7.32 (m, 2H), 4.36 (m, 2H), 4.04 (s, 3H), 3.86-3.74 (m, 7H), 3.51 (m, 2H), 3.09 (m, 2H), 2.71 (m, 1H), 1.28 (m, 3H). | 541.30 |
| 9YY | 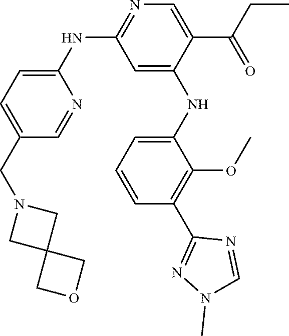 [SMILES: CCC(=O)c1cnc(Nc2ccc(CN3CC4(COC4)C3)cn2)cc1Nc1cccc(-c2ncn(C)n2)c1OC] | 1H NMR (300 MHz, Methanol-d4) δ 8.80 (m, 1H), 8.50 (m, 1H), 8.10 (m, 1H), 7.87 (m, 1H), 7.76-7.60 (m, 3H), 7.40-7.31 (m, 2H), 4.74 (m, 4H), 4.04 (s, 3H), 3.74 (s, 3H), 3.52 (m, 2H), 3.33 (m, 4H), 3.09 (m, 2H), 1.28 (m, 3H). | 541.25 |
| 9ZZ | 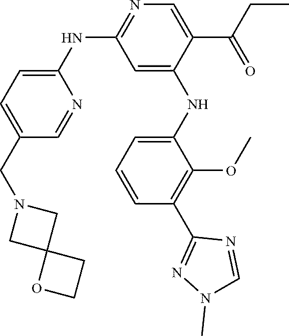 [SMILES: CCC(=O)c1cnc(Nc2ccc(CN3CC4(CCO4)C3)cn2)cc1Nc1cccc(-c2ncn(C)n2)c1OC] | 1H NMR (300 MHz, Methanol-d4) δ 8.77 (m, 1H), 8.48 (m, 1H), 8.08 (m, 1H), 7.84 (m, 1H), 7.73-7.56 (m, 3H), 7.38-7.29 (m, 2H), 4.49 (m, 2H), 4.02 (s, 3H), 3.72 (s, 3H), 3.55 (m, 4H), 3.30 (m, 2H), 3.07 (m, 2H), 2.83 (m, 2H), 1.24 (m, 3H). | 541.35 |
| 9AAA | 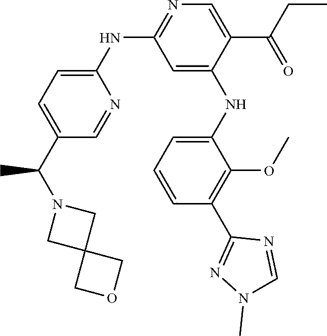 [SMILES: CCC(=O)c1cnc(Nc2ccc([C@H](C)N3CC4(COC4)C3)cn2)cc1Nc1cccc(-c2ncn(C)n2)c1OC] | 1H NMR (400 MHz, DMSO-d6) δ 11.12 (s, 1H), 9.94 (s, 1H), 8.85 (s, 1H), 8.58 (s, 1H), 8.05 (d, J = 2.3 Hz, 1H), 7.99 (s, 1H), 7.70 (d, J = 8.0 Hz, 1H), 7.63 (d, J = 7.8 Hz, 1H), 7.58 (d, J = 8.7, 2.3 Hz, 1H), 7.51 (d, J = 8.7 Hz, 1H), 7.33 (t, J = 7.9 Hz, 1H), 4.58 (m, 4H), 3.96 (s, 3H), 3.75 (s, 3H), 3.24 (d, J = 7.4 Hz, 2H), 3.17 (d, J = 5.9 Hz, 1H), 3.10 - 3.05 (m, 4H), 1.14 (t, J = 7.2 Hz, 3H), 1.09 (d, J = 6.4 Hz, 3H). | 555.3 |
| 9BBB | 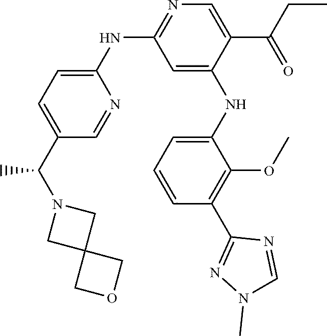 [SMILES: CCC(=O)c1cnc(Nc2ccc([C@@H](C)N3CC4(COC4)C3)cn2)cc1Nc1cccc(-c2ncn(C)n2)c1OC] | 1H NMR (300 MHz, Methanol-d4) δ 8.78 (s, 1H), 8.48 (s, 1H), 8.09 (d, J = 2.3 Hz, 1H), 7.82 (s, 1H), 7.78 -7.71 (m, 1H), 7.68 - 7.55 (m, 2H), 7.40 - 7.26 (m, 2H), 4.71 (m, 4H), 4.02 (s, 3H), 3.72 (s, 3H), 3.42 (d, J = 7.9 Hz, 2H), 3.26 (d, J = 8.0 Hz, 3H), 3.06 (q, J = 7.3 Hz, 2H), 1.31 -1.15 (m, 6H). | 555.3 |
| 9CCC | 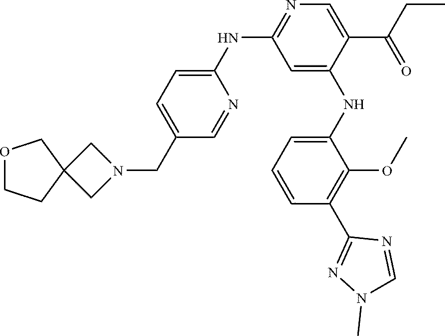 [SMILES: CCC(=O)c1cnc(Nc2ccc(CN3CC4(CCOC4)C3)cn2)cc1Nc1cccc(-c2ncn(C)n2)c1OC] | 1H NMR (400 MHz, Methanol-d4) δ 8.81 (m, 1H), 8.51 (m, 1H), 8.15 (m, 1H), 7.85 (m, 1H), 7.74 (m, 1H), 7.66 (m, 2H), 7.42-7.33 (m, 2H), 4.05 (s, 3H), 3.83-3.67 (m, 9H), 3.4 (m, 4H), 3.15 (m, 2H), 2.14 (m, 2H), 1.28 (m, 3H). | 555.30 |
| 9DDD | 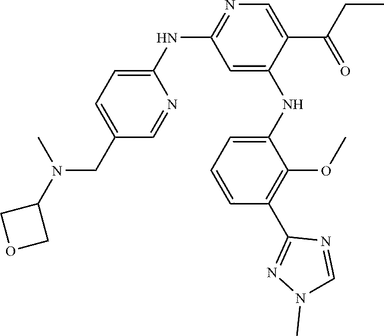 [SMILES: CCC(=O)c1cnc(Nc2ccc(CN(C)C3COC3)cn2)cc1Nc1cccc(-c2ncn(C)n2)c1OC] | 1H NMR (400 MHz, Methanol-d4) δ 8.80 (m, 1H), 8.50 (m, 1H), 8.11 (m, 1H), 7.84 (m, 1H), 7.75-7.65 (m, 3H), 7.43-7.31 (m, 2H), 4.88-4.56 (m, 4H), 4.04 (s, 3H), 3.74 (s, 3H), 3.68 (m, 1H), 3.40 (m, 2H), 3.10 (m, 2H), 2.09 (s, 3H), 1.26 (m, 3H). | 529.30 |
| 9EEE | 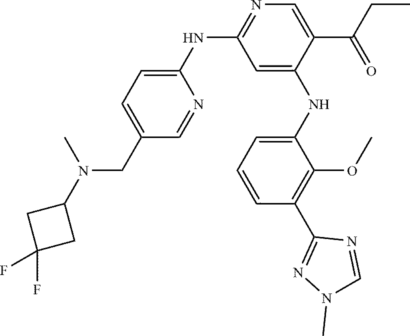 [SMILES: CCC(=O)c1cnc(Nc2ccc(CN(C)C3CC(F)(F)C3)cn2)cc1Nc1cccc(-c2ncn(C)n2)c1OC] | 1H NMR (400 MHz, Methanol-d4) δ 8.80 (m, 1H), 8.50 (m, 1H), 8.11 (m, 1H), 7.81 (m, 1H), 7.74-7.64 (m, 3H), 7.42-7.31 (m, 2H), 4.04 (s, 3H), 3.74 (s, 3H), 3.43 (m, 2H), 3.10 (m, 2H), 2.86 (m, 1H), 2.75 (m, 2H), 2.50 (m, 2H), 2.08 (s, 3H), 1.24 (m, 3H). | 563.35 |
| 9FFF | 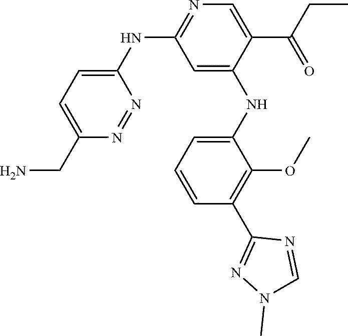 [SMILES: CCC(=O)c1cnc(Nc2ccc(CN)nn2)cc1Nc1cccc(-c2ncn(C)n2)c1OC] | 1H NMR (300 MHz, Methanol-d4) δ 8.98 (s, 1H), 8.54 (s, 1H), 7.89 (m, 1H), 7.83 (m, 1H), 7.59 (m, 1H), 7.40 (m, 2H), 6.64 (m, 1H), 4.46 (m, 2H), 4.03 (s, 3H), 3.71 (s, 3H), 3.18 (m, 2H), 1.27 (m, 3H). | 460.20 |
| 9GGG | 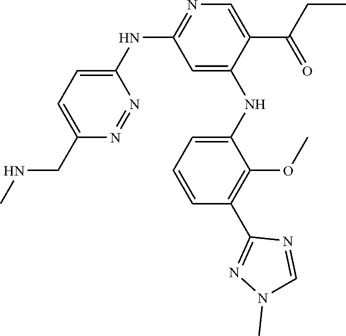 [SMILES: CCC(=O)c1cnc(Nc2ccc(CNC)nn2)cc1Nc1cccc(-c2ncn(C)n2)c1OC] | 1H NMR (300 MHz, Methanol-d4) δ 9.01 (s, 1H), 8.64 (s, 1H), 7.91 (m, 1H), 7.80 (m, 1H), 7.62 (m, 1H), 7.45 (m, 2H), 6.69 (m, 1H), 4.56 (m, 2H), 4.06 (s, 3H), 3.74 (s, 3H), 3.18 (m, 2H), 2.89 (m, 3H), 1.29 (m, 3H). | 474.25 |
| 9HHH | 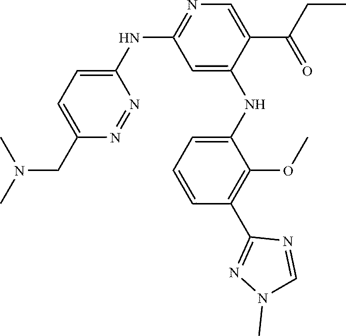 [SMILES: CCC(=O)c1cnc(Nc2ccc(CN(C)C)nn2)cc1Nc1cccc(-c2ncn(C)n2)c1OC] | 1H NMR (300 MHz, DMSO-d6) δ 11.10 (br s, 1H), 10.32 (br s, 1H), 8.88 (m, 1H), 8.57 (m, 1H), 8.10 (m, 1H), 7.65-7.57 (m, 4H), 7.27 (m, 1H), 3.96 (s, 3H), 3.76 (s, 3H), 3.59 (m, 2H), 3.12 (m, 2H), 2.17 (m, 6H), 1.13 (m, 3H). | 488.25 |
| 9III | 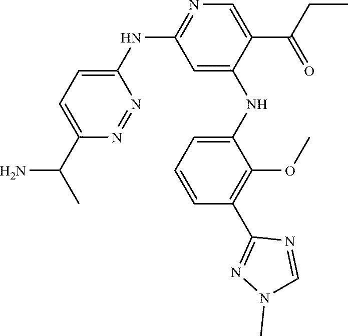 [SMILES: CCC(=O)c1cnc(Nc2ccc(C(C)N)nn2)cc1Nc1cccc(-c2ncn(C)n2)c1OC] | 1H NMR (300 MHz, DMSO-d6) δ 11.08 (br s, 1H), 10.25 (br s, 1H), 8.87 (m, 1H), 8.57 (m, 1H), 8.06 (m, 1H), 7.70-7.63 (m, 4H), 7.27 (m, 1H), 4.17 (m, 1H), 3.96 (s, 3H), 3.75 (s, 3H), 3.10 (m, 2H), 1.34 (m, 3H), 1.13 (m, 3H). | 474.15 |
| 9JJJ | 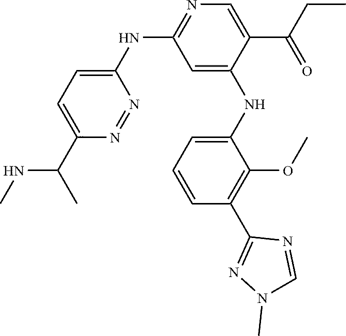 [SMILES: CCC(=O)c1cnc(Nc2ccc(C(C)NC)nn2)cc1Nc1cccc(-c2ncn(C)n2)c1OC] | 1H NMR (400 MHz, DMSO-d6) δ 11.09 (br s, 1H), 10.26 (br s, 1H), 8.87 (m, 1H), 8.57 (m, 1H), 8.08 (m, 1H), 7.66-7.60 (m, 4H), 7.28 (m, 1H), 3.96 (m, 3H), 3.84-3.73 (m, 4H), 3.11 (m, 2H), 2.14 (m, 3H), 1.26 (m, 3H), 1.15 (m, 3H). | 488.15 |
| 9KKK | 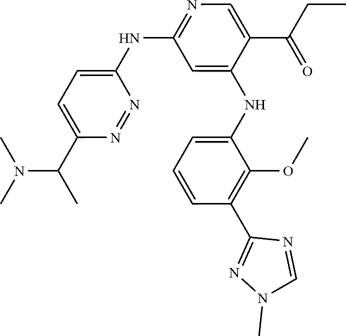 [SMILES: CCC(=O)c1cnc(Nc2ccc(C(C)N(C)C)nn2)cc1Nc1cccc(-c2ncn(C)n2)c1OC] | 1H NMR (300 MHz, DMSO-d6) δ 11.10 (br s, 1H), 10.30 (br s, 1H), 8.88 (m, 1H), 8.57 (m, 1H), 8.09 (m, 1H), 7.67-7.56 (m, 4H), 7.27 (m, 1H), 3.96 (m, 3H), 3.76 (s, 3H), 3.62 (m, 1H), 3.10 (m, 2H), 2.12 (s, 6H), 1.31 (m, 3H), 1.13 (m, 3H). | 502.15 |
| 9LLL | 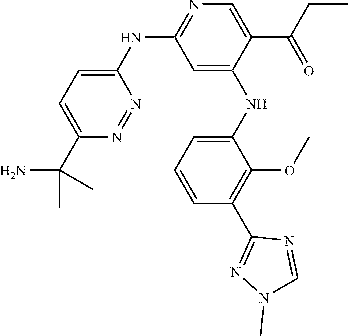 [SMILES: CCC(=O)c1cnc(Nc2ccc(C(C)(C)N)nn2)cc1Nc1cccc(-c2ncn(C)n2)c1OC] | 1H NMR (400 MHz, Methanol-d4) δ 8.85 (s, 1H), 8.51 (s, 1H), 8.20 (m, 1H), 7.78 (m, 1H), 7.67 (m, 2H), 7.32 (m, 2H), 4.04 (s, 3H), 3.74 (s, 3H), 3.10 (m, 2H), 1.57 (s, 6H), 1.28 (m, 3H). | 486.15 |
| 9MMM | 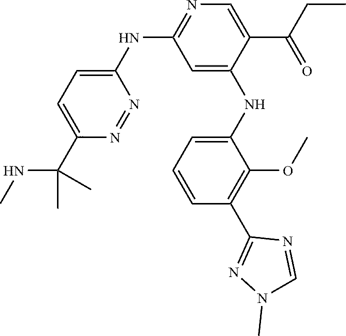 [SMILES: CCC(=O)c1cnc(Nc2ccc(C(C)(C)NC)nn2)cc1Nc1cccc(-c2ncn(C)n2)c1OC] | 1H NMR (300 MHz, Methanol-d4) δ 8.86 (s, 1H), 8.50 (s, 1H), 8.24 (m, 1H), 7.76-7.66 (m, 3H), 7.36-7.28 (m, 2H), 4.04 (s, 3H), 3.73 (s, 3H), 3.12 (m, 2H), 2.36 (m, 3H), 1.62 (s, 6H), 1.26 (m, 3H). | M-1 = 500.15 |
| 9NNN | 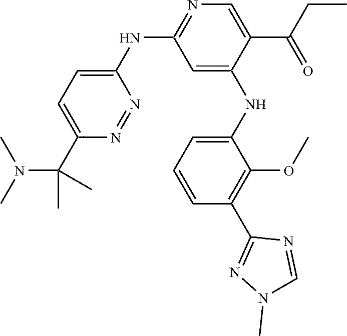 [SMILES: CCC(=O)c1cnc(Nc2ccc(C(C)(C)N(C)C)nn2)cc1Nc1cccc(-c2ncn(C)n2)c1OC] | 1H NMR (300 MHz, Methanol-d4) δ 9.01 (s, 1H), 8.63 (s, 1H), 8.01 (m, 1H), 7.92 (m, 1H), 7.64 (m, 1H), 7.51-7.40 (m, 2H), 6.72 (m, 1H), 4.06 (s, 3H), 3.74 (s, 3H), 3.21 (m, 2H), 2.91 (s, 6H), 1.84 (s, 6H), 1.29 (m, 3H). | 516.30 |
| 9OOO | 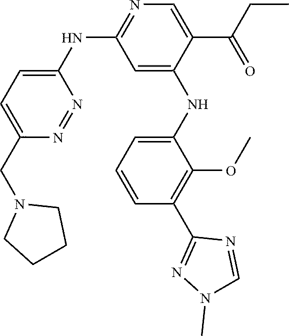 [SMILES: CCC(=O)c1cnc(Nc2ccc(CN3CCCC3)nn2)cc1Nc1cccc(-c2ncn(C)n2)c1OC] | 1H NMR (300 MHz, DMSO-d6) δ 11.07 (br s, 1H), 10.29 (br s, 1H), 8.86 (m, 1H), 8.55 (m, 1H), 8.06 (m, 1H), 7.64-7.54 (m, 4H), 7.24 (m, 1H), 3.94 (s, 3H), 3.74 (m, 5H), 3.11 (m, 2H), 2.5 (m, 4H), 1.68 (m, 4H), 1.12 (m, 3H). | 514.25 |
| 9PPP | 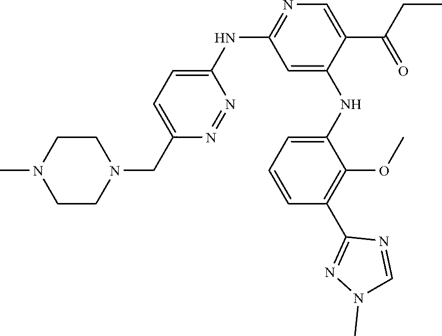 [SMILES: CCC(=O)c1cnc(Nc2ccc(CN3CCN(C)CC3)nn2)cc1Nc1cccc(-c2ncn(C)n2)c1OC] | 1H NMR (300 MHz, DMSO-d6) δ 11.08 (br s, 1H), 10.30 (br s, 1H), 8.86 (m, 1H), 8.56 (m, 1H), 8.07 (m, 1H), 7.63 (m, 3H), 7.54 (m, 1H), 7.26 (m, 1H), 3.94 (m, 3H), 3.74 (s, 3H), 3.63 (m, 2H), 3.08 (m, 2H), 2.4-2.2 (m, 8H), 2.13 (s, 3H), 1.12 (m, 3H). | 541.25 |
| 9QQQ | 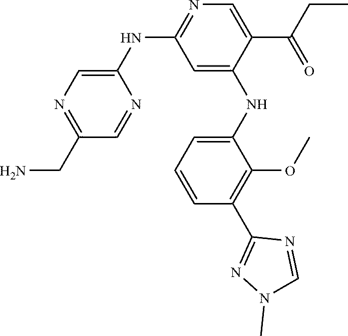 [SMILES: CCC(=O)c1cnc(Nc2cnc(CN)cn2)cc1Nc1cccc(-c2ncn(C)n2)c1OC] | 1H NMR (400 MHz, Methanol-d4) δ 8.84 (s, 1H), 8.82 (m, 1H), 8.50 (m, 1H), 8.21 (m, 1H), 7.76-7.66 (m, 3H), 7.33 (m, 1H), 4.04 (s, 3H), 3.89 (m, 2H), 3.74 (s, 3H), 3.09 (m, 2H), 1.26 (m, 3H). | 460.15 |
| 9RRR | 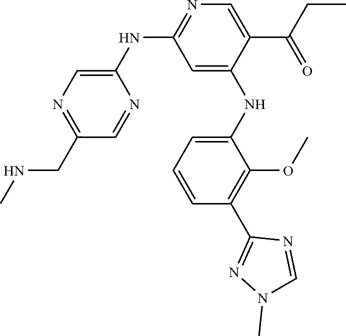 [SMILES: CCC(=O)c1cnc(Nc2cnc(CNC)cn2)cc1Nc1cccc(-c2ncn(C)n2)c1OC] | 1H NMR (400 MHz, Methanol-d4) δ 8.84 (m, 2H), 8.50 (m, 1H), 8.20 (m, 1H), 7.76-7.65 (m, 3H), 7.33 (m, 1H), 4.04 (s, 3H), 3.82 (m, 2H), 3.73 (s, 3H), 3.10 (m, 2H), 2.45 (m, 3H), 1.25 (m, 3H). | 474.20 |
| 9SSS | 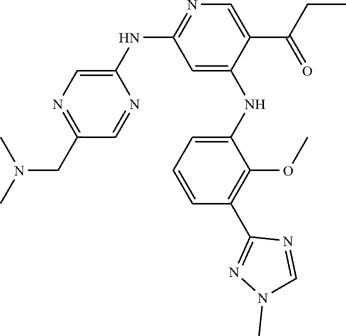 [SMILES: CCC(=O)c1cnc(Nc2cnc(CN(C)C)cn2)cc1Nc1cccc(-c2ncn(C)n2)c1OC] | 1H NMR (300 MHz, Methanol-d4) δ 8.83 (m, 2H), 8.49 (m, 1H), 8.19 (m, 1H), 7.75 (m, 1H), 7.70-7.63 (m, 2H), 7.31 (m, 1H), 4.02 (s, 3H), 3.7 (m, 5H), 3.06 (m, 2H), 2.38 (s, 6H), 1.23 (m, 3H). | 488.15 |
| 9TTT | 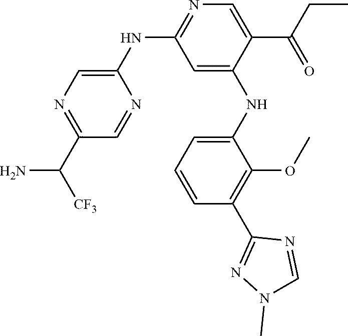 [SMILES: CCC(=O)c1cnc(Nc2cnc(C(N)C(F)(F)F)cn2)cc1Nc1cccc(-c2ncn(C)n2)c1OC] | 1H NMR (400 MHz, Methanol-d4) δ 8.93 (m, 1H), 8.59-8.53 (m, 3H), 7.91 (m, 1H), 7.62 (m, 1H), 7.42 (m, 1H), 6.79 (m, 1H), 5.68 (m, 1H), 4.05 (s, 3H), 3.74 (s, 3H), 3.17 (m, 2H), 1.29 (m, 3H). | 528.2 |
| 9UUU | 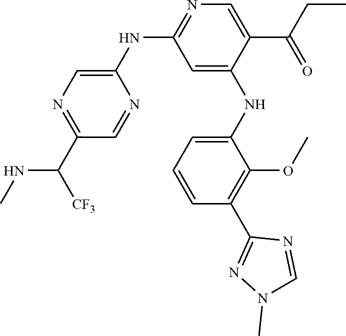 [SMILES: CCC(=O)c1cnc(Nc2cnc(C(NC)C(F)(F)F)cn2)cc1Nc1cccc(-c2ncn(C)n2)c1OC] | 1H NMR (400 MHz, Methanol-d4) δ 8.94 (m, 1H), 8.59-8.55 (m, 3H), 7.90 (m, 1H), 7.62 (m, 1H), 7.42 (m, 1H), 6.82 (m, 1H), 5.58 (m, 1H), 4.05 (s, 3H), 3.74 (s, 3H), 3.17 (m, 2H), 2.75 (m, 3H), 1.29 (m, 3H). | 542.20 |
| 9VVV | 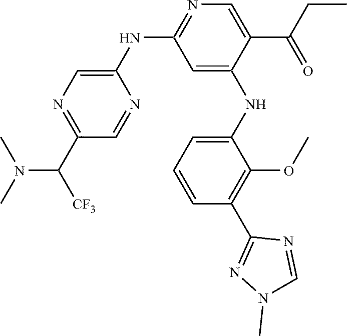 [SMILES: CCC(=O)c1cnc(Nc2cnc(C(N(C)C)C(F)(F)F)cn2)cc1Nc1cccc(-c2ncn(C)n2)c1OC] | 1H NMR (400 MHz, Methanol-d4) δ 8.92 (m, 1H), 8.87 (m, 1H), 8.51 (m, 1H), 8.23 (m, 1H), 7.70 (m, 3H), 7.34 (m, 1H), 4.42 (m, 1H), 4.04 (s, 3H), 3.74 (s, 3H), 3.12 (m, 2H), 2.42 (s, 6H), 1.26 (m, 3H). | 556.20 |
| 9WWW | 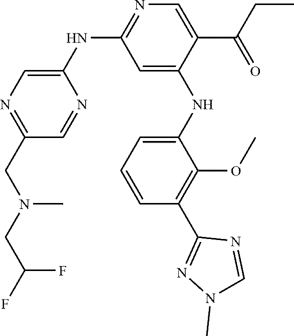 [SMILES: CCC(=O)c1cnc(Nc2cnc(CN(C)CC(F)F)cn2)cc1Nc1cccc(-c2ncn(C)n2)c1OC] | 1H NMR (300 MHz, Methanol-d4) δ 8.82 (m, 2H), 8.47 (m, 1H), 8.22 (m, 1H), 7.67 (m, 3H), 7.31 (m, 1H), 5.95 (m, 1H), 4.02 (s, 3H), 3.72 (m, 5H), 3.09 (m, 2H), 2.85 (m, 2H), 2.38 (s, 3H), 1.26 (m, 3H). | 538.35 |
| 9XXX | 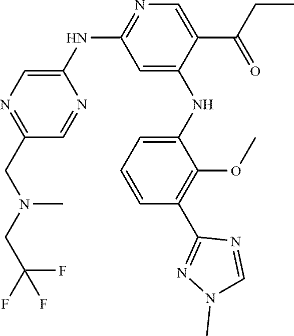 [SMILES: CCC(=O)c1cnc(Nc2cnc(CN(C)CC(F)(F)F)cn2)cc1Nc1cccc(-c2ncn(C)n2)c1OC] | 1H NMR (400 MHz, Methanol-d4) δ 8.92 (s, 1H), 8.62 (s, 1H), 8.47 (m, 1H), 8. 35(m, 1H), 7.89 (m, 1H), 7.62 (m, 1H), 7.41 (m, 1H), 6.64 (m, 1H), 4.06 (s, 3H), 4.01 (m, 2H), 3.74 (s, 3H), 3.35 (m, 2H), 3.18 (m, 2H), 2.56 (s, 3H), 1.29 (m, 3H). | 556.20 |
| 9YYY | 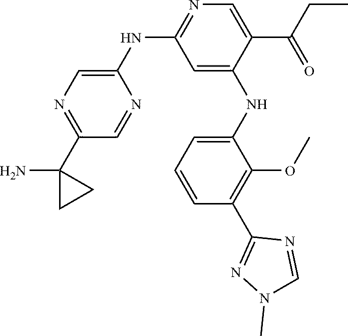 [SMILES: CCC(=O)c1cnc(Nc2cnc(C3(N)CC3)cn2)cc1Nc1cccc(-c2ncn(C)n2)c1OC] | 1H NMR (300 MHz, DMSO-d6) δ 11.10 (s, 1H), 10.13 (s, 1H), 8.87 (m, 1H), 8.81 (m, 1H), 8.57 (m, 1H), 8.53 (m, 1H), 7.75-7.62 (m, 3H), 7.32 (m, 1H), 3.96 (s, 3H), 3.75 (s, 3H), 3.08 (m, 2H), 1.13 (m, 5H), 0.93 (m, 2H). | 486.15 |
| 9ZZZ | 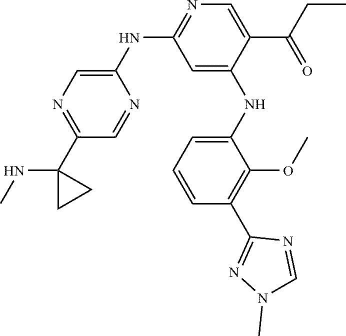 [SMILES: CCC(=O)c1cnc(Nc2cnc(C3(NC)CC3)cn2)cc1Nc1cccc(-c2ncn(C)n2)c1OC] | 1H NMR (300 MHz, DMSO-d6) δ 11.09 (s, 1H), 10.11 (s, 1H), 8.87 (m, 2H), 8.57 (m, 1H), 8.38 (m, 1H), 7.69-7.63 (m, 3H), 7.31 (m, 1H), 3.98 (s, 3H), 3.77 (s, 3H), 3.09 (m, 2H), 2.66 (m, 1H), 2.27 (m, 3H), 1.12 (m, 5H), 0.95 (m, 2H). | 500.3 |
| 9AAAA | 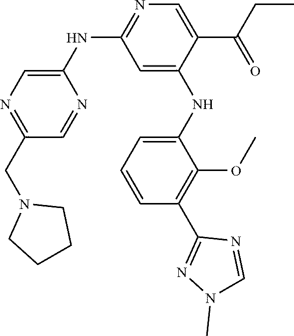 [SMILES: CCC(=O)c1cnc(Nc2cnc(CN3CCCC3)cn2)cc1Nc1cccc(-c2ncn(C)n2)c1OC] | 1H NMR (400 MHz, DMSO-d6) δ 11.09 (br s, 1H), 10.21 (br s, 1H), 8.90 (m, 2H), 8.57 (m, 1H), 8.17 (m, 1H), 7.72 (m, 1H), 7.64 (m, 2H), 7.31 (m, 1H), 3.95 (s, 3H), 3.74 (s, 3H), 3.64 (m, 2H), 3.10 (m, 2H), 2.51 (m, 4H), 1.69 (m, 4H), 1.14 (m, 3H). | 514.35 |
| 9BBBB | 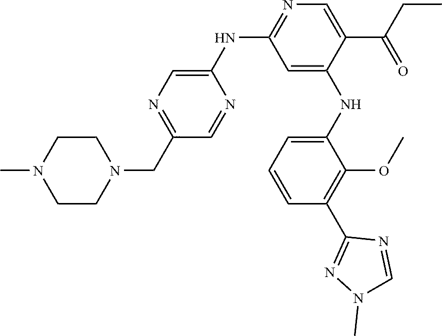 [SMILES: CCC(=O)c1cnc(Nc2cnc(CN3CCN(C)CC3)cn2)cc1Nc1cccc(-c2ncn(C)n2)c1OC] | 1H NMR (400 MHz, Methanol-d4) δ 8.80 (m, 2H), 8.48 (m, 1H), 8.19 (m, 1H), 7.71-7.64 (m, 3H), 7.30 (m, 1H), 4.02 (s, 3H), 3.71 (s, 3H), 3.60 (m, 2H), 3.07 (m, 2H), 2.6-2.4 (m, 8H), 2.27 (s, 3H), 1.23 (m, 3H). | 543.25 |
| 9CCCC | 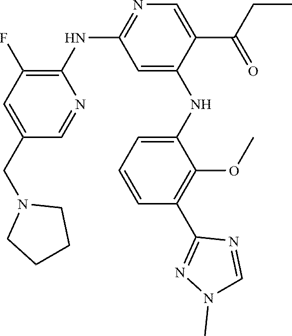 [SMILES: CCC(=O)c1cnc(Nc2ncc(CN3CCCC3)cc2F)cc1Nc1cccc(-c2ncn(C)n2)c1OC] | 1H NMR (300 MHz, Methanol-d4) δ 8.80 (m, 1H), 8.49 (m, 1H), 8.35 (m, 1H), 7.99 (m, 1H), 7.78 (m, 1H), 7.66 (m, 1H), 7.54 (m, 1H), 7.34 (m, 1H), 4.04 (s, 3H), 3.76 (s, 3H), 3.65 (m, 2H), 3.13 (m, 2H), 2.59 (m, 4H), 1.88 (m, 4H), 1.26 (m, 3H). | 531.45 |
| 9DDDD | 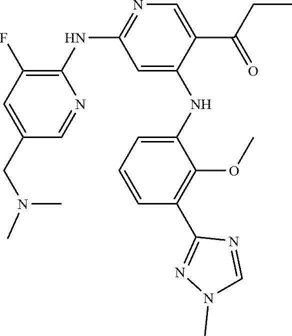 [SMILES: CCC(=O)c1cnc(Nc2ncc(CN(C)C)cc2F)cc1Nc1cccc(-c2ncn(C)n2)c1OC] | 1H NMR (400 MHz, Methanol-d4) δ 8.80 (m, 1H), 8.49 (m, 1H), 8.35 (m, 1H), 7.95 (m, 1H), 7.78 (m, 1H), 7.67 (m, 1H), 7.54 (m, 1H), 7.34 (m, 1H), 4.04 (s, 3H), 3.75 (s, 3H), 3.47 (m, 2H), 3.10 (m, 2H), 2.27 (s, 6H), 1.26 (m, 3H). | 505.35 |
| 9EEEE | 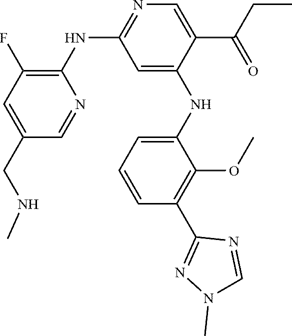 [SMILES: CCC(=O)c1cnc(Nc2ncc(CNC)cc2F)cc1Nc1cccc(-c2ncn(C)n2)c1OC] | 1H NMR (300 MHz, Methanol-d4) δ 8.79 (m, 1H), 8.49 (m, 1H), 8.35 (m, 1H), 7.99 (m, 1H), 7.76 (m, 1H), 7.67 (m, 1H), 7.56 (m, 1H), 7.34 (m, 1H), 4.04 (s, 3H), 3.79-3.75 (m, 5H), 3.08 (m, 2H), 2.39 (m, 3H), 1.26 (m, 3H). | 491.25 |
| 9FFFF | 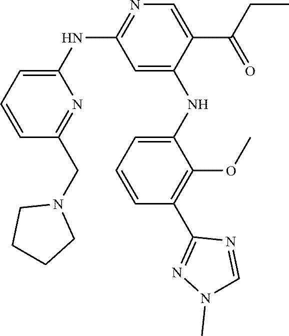 [SMILES: CCC(=O)c1cnc(Nc2cccc(CN3CCCC3)n2)cc1Nc1cccc(-c2ncn(C)n2)c1OC] | 1H NMR (400 MHz, DMSO-d6) δ 11.03 (br s, 1H), 9.92 (br s, 1H), 8.82 (s, 1H), 8.56 (m, 1H), 8.21 (m, 1H), 7.69-7.57 (m, 3H), 7.30 (m, 1H), 7.20 (m, 1H), 6.89 (m, 1H), 3.95 (s, 3H), 3.72 (s, 3H), 3.44 (s, 2H), 3.07 (m, 2H), 2.35 (m, 4H), 1.61 (m, 4H), 1.13 (m, 3H). | 513.35 |
| 9GGGG | 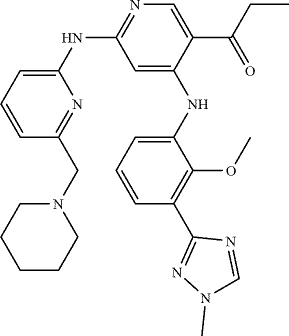 [SMILES: CCC(=O)c1cnc(Nc2cccc(CN3CCCCC3)n2)cc1Nc1cccc(-c2ncn(C)n2)c1OC] | 1H NMR (300 MHz, Methanol-d4) δ 8.79 (s, 1H), 8.50 (s, 1H), 8.32 (m, 1H), 7.78 (m, 1H), 7.70 (m, 1H), 7.63 (m, 2H), 7.37 (m, 1H), 7.06 (m, 1H), 6.98 (m, 1H), 4.04 (s, 3H), 3.75 (s, 3H), 3.55 (s, 2H), 3.09 (m, 2H), 2.33 (m, 4H), 1.55 (m, 4H), 1.36 (m, 2H), 1.25 (m, 3H). | 527.25 |
| 9HHHH | 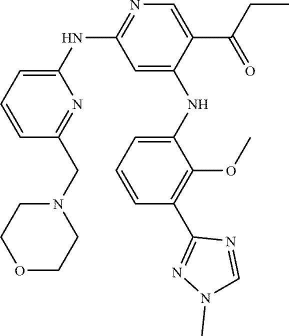 [SMILES: CCC(=O)c1cnc(Nc2cccc(CN3CCOCC3)n2)cc1Nc1cccc(-c2ncn(C)n2)c1OC] | 1H NMR (300 MHz, DMSO-d6) δ 11.04 (br s, 1H), 9.94 (br s, 1H), 8.83 (s, 1H), 8.56 (m, 1H), 8.16 (m, 1H), 7.70-7.59 (m, 3H), 7.34-7.24 (m, 2H), 6.93 (m, 1H), 3.91 (s, 3H), 3.73 (s, 3H), 3.53 (m, 4H), 3.3 (m, 2H), 3.11 (m, 2H), 2.31 (m, 4H), 1.16 (m, 3H). | 529.35 |
| 9IIII | 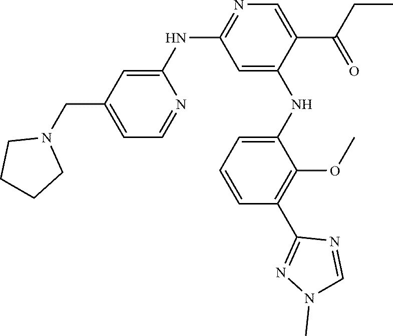 [SMILES: CCC(=O)c1cnc(Nc2cc(CN3CCCC3)ccn2)cc1Nc1cccc(-c2ncn(C)n2)c1OC] | 1H NMR (300 MHz, Methanol-d4) δ 8.79 (s, 1H), 8.48 (s, 1H), 8.15 (m, 1H), 7.82-7.63 (m, 3H), 7.38-7.28 (m, 2H), 6.94 (m, 1H), 4.24 (s, 3H), 3.73 (s, 3H), 3.64 (m, 2H), 3.08 (m, 2H), 2.61 (m, 4H), 1.84 (m, 4H), 1.26 (m, 3H). | 513.20 |
| 9JJJJ | 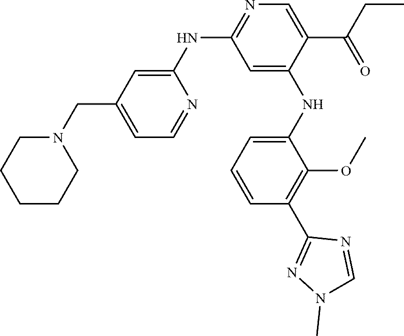 [SMILES: CCC(=O)c1cnc(Nc2cc(CN3CCCCC3)ccn2)cc1Nc1cccc(-c2ncn(C)n2)c1OC] | 1H NMR (300 MHz, Methanol-d4) δ 8.78 (s, 1H), 8.48 (s, 1H), 8.11 (m, 1H), 7.81 (m, 1H), 7.71 (m, 1H), 7.65 (m, 1H), 7.33-7.27 (m, 2H), 6.92 (m, 1H), 4.02 (s, 3H), 3.72 (s, 3H), 3.46 (m, 2H), 3.05 (m, 2H), 2.43 (m, 4H), 1.59 (m, 4H), 1.46 (m, 2H), 1.22 (m, 3H). | 527.40 |
| 9KKKK | 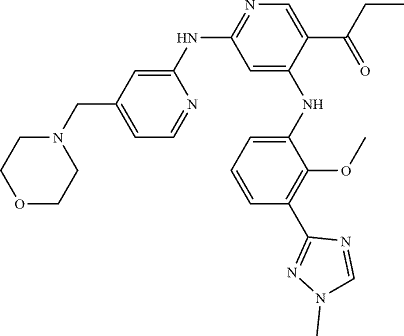 [SMILES: CCC(=O)c1cnc(Nc2cc(CN3CCOCC3)ccn2)cc1Nc1cccc(-c2ncn(C)n2)c1OC] | 1H NMR (400 MHz, DMSO-d6) δ 11.11 (br s, 1H), 9.92 (br s, 1H), 8.85 (m, 1H), 8.57 (m, 1H), 8.11 (m, 1H), 8.03 (m, 1H), 7.70 (m, 1H), 7.61 (m, 1H), 7.53 (m, 1H), 7.31 (m, 1H), 6.87 (m, 1H), 3.96 (s, 3H), 3.75 (s, 3H), 3.60 (m, 4H), 3.44 (m, 2H), 3.11 (m, 2H), 2.38 (m, 4H), 1.14 (m, 3H). | 529.25 |
| 9LLLL | 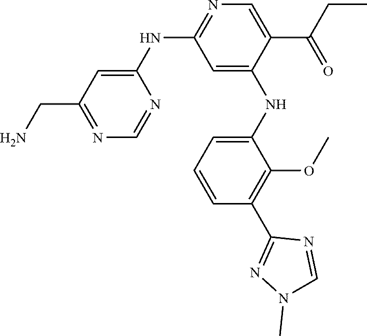 [SMILES: CCC(=O)c1cnc(Nc2cc(CN)ncn2)cc1Nc1cccc(-c2ncn(C)n2)c1OC] | 1H NMR (300 MHz, DMSO-d6) δ 11.07 (br s, 1H), 9.78 (br s, 1H), 8.84 (m, 1H), 8.57 (m, 1H), 8.12 (m, 1H), 7.67-7.61 (m, 3H), 7.32 (m, 1H), 7.09 (m, 1H), 6.81 (m, 1H), 3.96 (s, 3H), 3.76 (s, 3H), 3.11 (m, 2H), 2.76 (m, 3H), 1.15 (m, 3H). | 460.20 |
| 9MMMM | 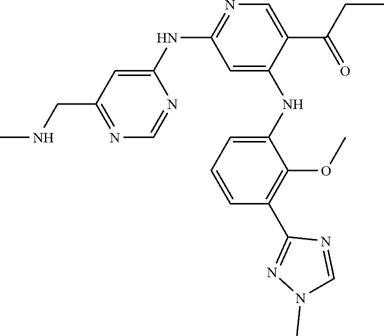 [SMILES: CCC(=O)c1cnc(Nc2cc(CNC)ncn2)cc1Nc1cccc(-c2ncn(C)n2)c1OC] | 1H NMR (300 MHz, Methanol-d4) δ 8.83 (s, 1H), 8.50 (s, 1H), 8.15 (m, 1H), 7.67 (m, 2H), 7.51 (m, 1H), 7.31 (m, 1H), 6.85 (m, 1H), 4.6 (br s, 1H), 4.04 (s, 3H), 3.73 (s, 3H), 3.15-3.05 (m, 7H), 1.23-1.24 (m, 3H). | 474.10 |
| 9NNNN | 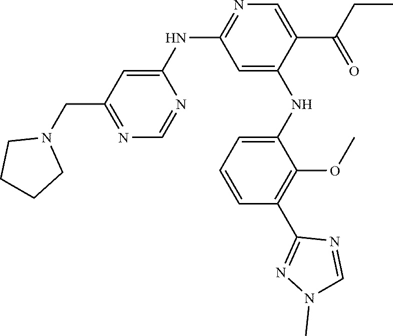 [SMILES: CCC(=O)c1cnc(Nc2cc(CN3CCCC3)ncn2)cc1Nc1cccc(-c2ncn(C)n2)c1OC] | 1H NMR (400 MHz, DMSO-d6) δ 11.10 (br s, 1H), 10.30 (br s, 1H), 8.90 (m, 1H), 8.58 (m, 2H), 7.92 (m, 1H), 7.71-7.64 (m, 3H), 7.33 (m, 1H), 3.96 (s, 3H), 3.78 (s, 3H), 3.60 (m, 2H), 3.13 (m, 2H), 2.5 (m, 4H), 1.74 (m, 4H), 1.14 (m, 3H). | 514.30 |
| 9OOOO | 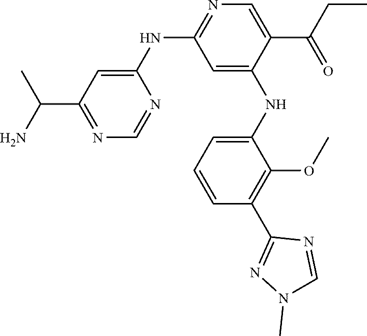 [SMILES: CCC(=O)c1cnc(Nc2cc(C(C)N)ncn2)cc1Nc1cccc(-c2ncn(C)n2)c1OC] | 1H NMR (300 MHz, DMSO-d6) δ 11.10 (br s, 1H), 10.31 (br s, 1H), 8.91 (m, 1H), 8.58 (m, 2H), 7.88 (m, 1H), 7.76 (m, 1H), 7.66 (m, 2H), 7.33 (m, 1H), 3.96 (m, 3H), 3.86 (m, 1H), 3.75 (s, 3H), 3.14 (m, 2H), 1.26 (m, 3H), 1.14 (m, 3H). | 474.15 |
| 9PPPP | 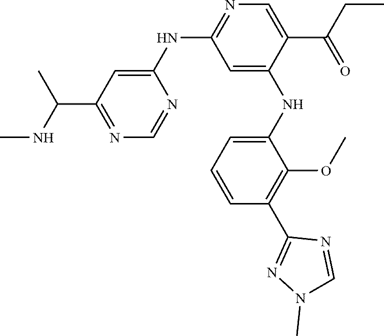 [SMILES: CCC(=O)c1cnc(Nc2cc(C(C)NC)ncn2)cc1Nc1cccc(-c2ncn(C)n2)c1OC] | 1H NMR (300 MHz, DMSO-d6) δ 11.10 (br s, 1H), 10.31 (br s, 1H), 8.92 (m, 1H), 8.62 (m, 1H), 8.57 (m, 1H), 7.86 (m, 1H), 7.71-7.62 (m, 3H), 7.33 (m, 1H), 3.96 (m, 3H), 3.75 (s, 3H), 3.48 (m, 1H), 3.14 (m, 2H), 2.19 (m, 3H), 1.23 (m, 3H), 1.14 (m, 3H). | 488.20 |
| 9QQQQ | 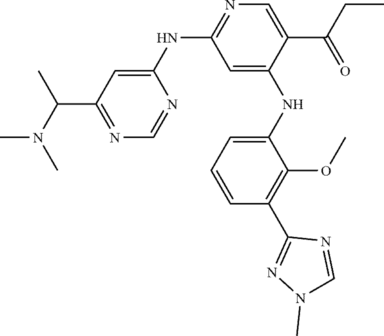 [SMILES: CCC(=O)c1cnc(Nc2cc(C(C)N(C)C)ncn2)cc1Nc1cccc(-c2ncn(C)n2)c1OC] | 1H NMR (300 MHz, DMSO-d6) δ 11.09 (br s, 1H), 10.29 (br s, 1H), 8.91 (m, 1H), 8.62 (m, 1H), 8.57 (m, 1H), 7.86 (m, 1H), 7.72-7.63 (m, 3H), 7.33 (m, 1H), 3.96 (m, 3H), 3.75 (s, 3H), 3.15 (m, 2H), 2.17 (m, 6H), 1.26 (m, 3H), 1.13 (m, 3H). | 502.25 |
| 9RRRR | 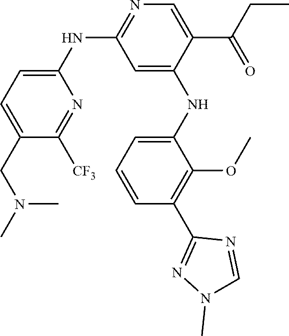 [SMILES: CCC(=O)c1cnc(Nc2ccc(CN(C)C)c(C(F)(F)F)n2)cc1Nc1cccc(-c2ncn(C)n2)c1OC] | 1H NMR (400 MHz, Methanol-d4) δ 8.83 (s, 1H), 8.49 (s, 1H), 7.96-7.93 (m, 2H), 7.69-7.56 (m, 3H), 7.27 (m, 1H), 4.04 (s, 3H), 3.74 (s, 3H), 3.55 (m, 2H), 3.09 (m, 2H), 2.26 (s, 6H), 1.26 (m, 3H). | 555.25 |
| 9SSSS | 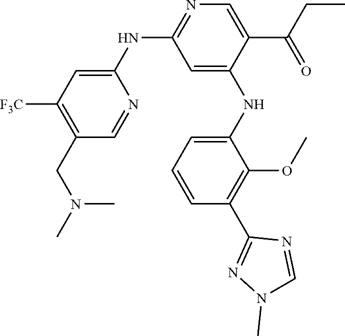 [SMILES: CCC(=O)c1cnc(Nc2cc(C(F)(F)F)c(CN(C)C)cn2)cc1Nc1cccc(-c2ncn(C)n2)c1OC] | 1H NMR (400 MHz, Methanol-d4) δ 8.86 (s, 1H), 8.51 (s, 1H), 8.47 (m, 1H), 7.96 (m, 1H), 7.76-7.61 (m, 3H), 7.35 (m, 1H), 4.05 (s, 3H), 3.74 (s, 3H), 3.57 (m, 2H), 3.12 (m, 2H), 2.29 (s, 6H), 1.26 (m, 3H). | 555.15 |
| 9TTTT | 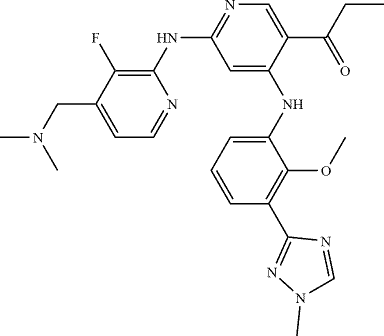 [SMILES: CCC(=O)c1cnc(Nc2nccc(CN(C)C)c2F)cc1Nc1cccc(-c2ncn(C)n2)c1OC] | 1H NMR (300 MHz, Methanol-d4) δ 8.87 (m, 1H), 8.54 (m, 1H), 8.36 (m, 1H), 8.02 (m, 1H), 7.77 (m, 1H), 7.67 (m, 1H), 7.36 (m, 1H), 7.04 (m, 1H), 4.04 (s, 3H), 3.75 (s, 3H), 3.60 (m, 2H), 3.14 (m, 2H), 2.31 (s, 6H), 1.27 (m, 3H). | 505.30 |
| 9UUUU | 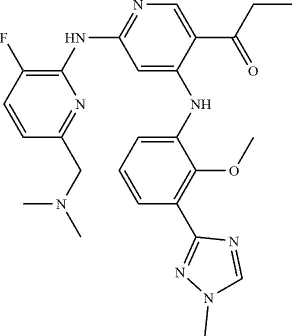 [SMILES: CCC(=O)c1cnc(Nc2nc(CN(C)C)ccc2F)cc1Nc1cccc(-c2ncn(C)n2)c1OC] | 1H NMR (400 MHz, Methanol-d4) δ 9.05 (s, 1H), 8.57 (s, 1H), 7.91 -7.85 (m, 2H), 7.60 - 7.58 (m, 1H), 7.47 - 7.36 (m, 2H), 6.95 (s, 1H), 4.53 (s, 2H), 4.05 (s, 3H), 3.72 (s, 3H), 3.19 (q, J = 7.1 Hz, 2H), 2.95 (s, 6H), 1.30 (t, J = 7.1 Hz, 3H). | 505.3 |

### Example 10

Preparation of 1-(4-((2-methoxy-3-(1-methyl-1H-1,2,4-triazol-3-yl)phenyl)amino)-6-((5-(2-(pyrrolidin-1-yl)ethoxy)pyridin-2-yl)amino )pyridin-3-yl)propan-1-one (Compound 10A)

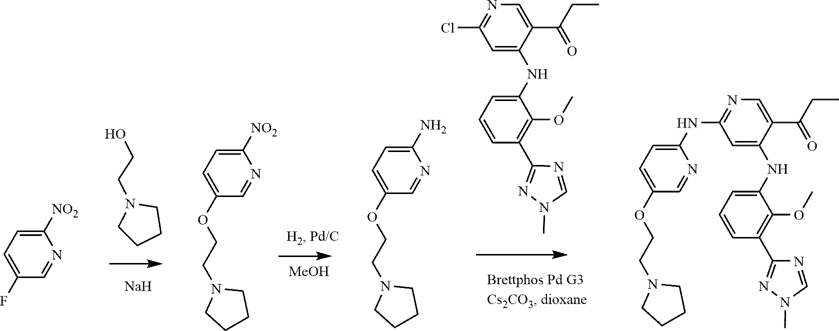

### Step 1. Synthesis of 2-nitro-5-(2-(pyrrolidin-1-yl)ethoxy)pyridine

To a solution of 5-fluoro-2-nitro-pyridine (300 mg, 2.11 mmol) in tetrahydrofuran (15 mL) was added sodium hydride (254 mg, 6.62 mmol, 60%) in portions at 0° C. The resulting solution was stirred for 40 min at 0° C. To this was added 2-pyrrolidin-1-ylethanol (292 mg, 2.53 mmol) dropwise. The resulting solution was stirred overnight at room temperature. The reaction was then quenched by the addition of saturated ammonium chloride, extracted with ethyl acetate (3 × 20 mL), washed with brine, dried over anhydrous sodium sulfate and concentrated under vacuum. The crude product was purified by prep-TLC (dichloromethane:ammonia in methanol; 6:1) to yield 2-nitro-5-(2-pyrrolidin-1-ylethoxy)pyridine (352 mg, 70%) as a solid.

### Step 2. Synthesis of 5-(2-(pyrrolidin-1-yl)ethoxy)pyridin-2-amine

A solution of 2-nitro-5-(2-pyrrolidin-1-ylethoxy)pyridine (300 mg, 1.26 mmol) in methanol (10 mL) was sparged with nitrogen for a few minutes before adding 10% palladium on carbon (208 mg, 0.04 mmol), sparged with nitrogen for a few minutes then allowing the mixture to stir under a balloon of hydrogen for 4 hours at room temperature. The mixture was then sparged with nitrogen and filtered through a pad of celite, washing with additional amounts of methanol. Concentrated and purified by prep-TLC (dichloromethane:methanol; 15:1) to yield 5-(2-pyrrolidin-1-ylethoxy)pyridin-2-amine (220 mg, 84%) as a solid.

### Step 3: 1-(4-((2-methoxy-3-(1-methyl-1H-1,2,4-triazol-3-yl)phenyl)amino)-6-((5-(2-(pyrrolidin-1-yl)ethoxy)pyridin-2-yl)amino)pyridin-3-yl)propan-1-one

To a solution of 1-(6-chloro-4-((2-methoxy-3-(1-methyl-1H-1,2,4-triazol-3-yl)phenyl)amino)pyridin-3-yl)propan-1-one (100 mg, 0.27 mmol) in 1,4-dioxane (10 mL) was added 5-(2-pyrrolidin-1-ylethoxy)pyridin-2-amine (111 mg, 0.54 mmol), Brettphos (29 mg, 0.054 mmol), Brettphos Pd G3 (24 mg, 0.03 mmol) and cesium carbonate (175 mg, 0.54 mmol). The reaction mixture was stirred for 4 hours at 100° C. under N2. After cooling, the mixture was filtered and the filtrate was concentrated. The residue was purified by prep-HPLC to yield 1-(4-((2-methoxy-3-(1-methyl-1H-1,2,4-triazol-3-yl)phenyl)amino)-6-((5-(2-(pyrrolidin-1-yl)ethoxy)pyridin-2-yl)amino)pyridin-3-yl)propan-1-one (62 mg, 42%) as a solid.

$$\left(\text{ES,} \text{m} / \text{z}\right) : \left[\text{M+H}\right] + = 543.35 .$$

¹H-NMR: (Methanol-d4, 300 MHz, ppm):8.77 (d, J = 1.0 Hz, 1H), 8.50 (s, 1H), 7.94 (d, J = 2.3 Hz, 1H), 7.72 (dd, J = 8.0, 1.6 Hz, 1H), 7.68 - 7.60 (m, 2H), 7.39 (q, J = 1.2 Hz, 2H), 7.37 - 7.27 (m, 1H), 4.22 - 4.12 (m, 2H), 4.04 (s, 3H), 3.74 (s, 3H), 3.07 (q, J = 7.3 Hz, 2H), 2.95 (d, J = 4.4 Hz, 2H), 2.71 (s, 4H), 1.87 (s, 4H), 1.25 (t, J = 7.3 Hz, 3H). LC-MS:

### Example 11

Preparation of 1-(6-((5-(2-( dimethylamino )ethyl)pyridin-2-yl)amino)-4-((2-methoxy-3-(1-methyl-1H-1,2,4-triazol-3-yl)phenyl)amino )pyridin-3-yl)propan-1-one (Compound 11A

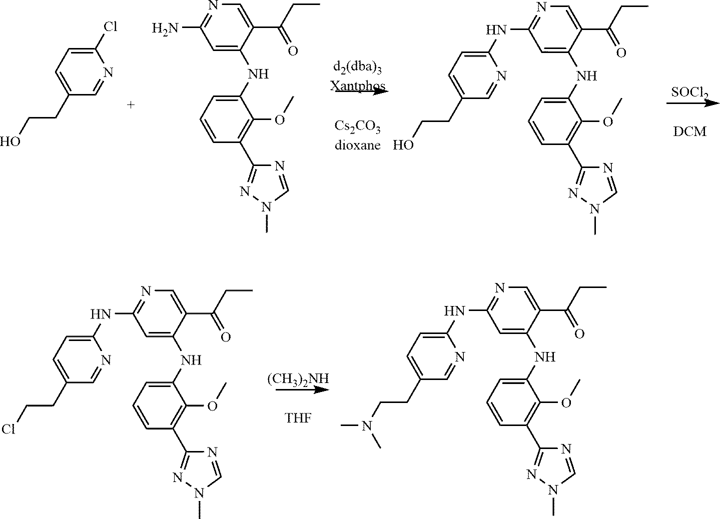

### Step 1: 1-(6-((5-(2-hydroxyethyl)pyridin-2-yl)amino)-4-((2-methoxy-3-(1-methyl-1H-1,2,4-triazol-3-yl)phenyl)amino)pyridin-3-yl)propan-1-one

1-(6-amino-4-((2-methoxy-3-(1-methyl-1H-1,2,4-triazol-3-yl)phenyl) amino)pyridin-3-yl)propan-1-one (179 mg, 1.1 mmol) and cesium carbonate (740 mg, 2.3 mmol) was suspended in 1,4-dioxane (20 mL), added XantPhos (131 mg, 0.22 mmol) and Pd₂(dba)₃ (104 mg, 0.11 mmol). The mixture was stirred at 100° C. under nitrogen overnight. The mixture was concentrated and purified with silica gel chromatography, eluting with dichloromethane:methanol (15:1) to yield 1-(6-((5-(2-hydroxyethyl)pyridin-2-yl) amino)-4-((2-methoxy-3-( 1-methyl-1H-1, 2, 4-triazol-3-yl)phenyl)amino)pyridin-3-yl)propan-1-one (220 mg, 41%) as a solid.

### Step 2: 1-(6-((5-(2-chloroethyl)pyridin-2-yl)amino)-4-((2-methoxy-3-(1-methyl-1H-1,2,4-triazol-3-yl)phenyl)amino)pyridin-3-yl)propan-1-one

1-(6-((5-(2-hydroxyethyl)pyridin-2-yl)amino)-4-((2-methoxy-3-(1-methyl-1H-1,2,4-triazol-3-yl)phenyl)amino )pyridin-3-yl)propan-1-one (220 mg, 0.46 mmol) was suspended in thionyl chloride (166 mg, 1.39 mmol) and dichloromethane (10 mL) and stirred at 50° C. under nitrogen for 2 hours. The reaction mixture was concentrated and purified with silica gel chromatography, eluting with dichloromethane:methanol (15:1) to yield 1-(6-((5-(2-chloroethyl)pyridin-2-yl)amino)-4-((2-methoxy-3-(1-methyl-1H-1,2,4-triazol-3-yl)phenyl)amino )pyridin-3-yl)propan-1-one (200 mg, 88% yield) as a solid.

### Step 3: 1-(6-((5-(2-(Dimethylamino)ethyl)pyridin-2-yl)amino)-4-((2-methoxy-3-(1-methyl-1H-1,2,4-triazol-3-yl)phenyl)amino)pyridin-3-yl)propan-1-one

1-(6-((5-(2-chloroethyl)pyridin-2-yl)amino)-4-((2-methoxy-3-(1-methyl-1H-1,2,4-triazol-3-yl)phenyl)amino)pyridin-3-yl)propan-1-one (60 mg, 0.12 mmol) was suspended in 2 M dimethylamine in tetrahydrofuran (8 mL) and stirred at 90° C. for over 2 days. The reaction mixture was concentrated and purified with silica gel chromatography, eluting with dichloromethane:methanol (15:1). The crude product was repurified by prep-HPLC to yield 1-(6-((5-(2-(dimethylamino)ethyl)pyridin-2-yl)amino)-4-((2-methoxy-3-(1-methyl-1H-1,2,4-triazol-3-yl)phenyl)amino)pyridin-3-yl)propan-1-one (26.3 mg, 43%) as a solid. ¹H NMR (300 MHz, Methanol-d₄) δ 8.93 (s, 1H), 8.55 (s, 1H), 8.34 (d, J= 2.3 Hz, 1H), 7.88-7.82 (m, 2H), 7.57 (d, J = 7.9 Hz, 1H), 7.38 (t, J = 7.9 Hz, 1H), 7.02 (d, J = 8.6 Hz, 1H), 6.49 (s, 1H), 4.03 (s, 3H), 3.71 (s, 3H), 3.45-3.31 (m, 2H), 3.21-3.04 (m, 4H), 2.97 (s, 6H), 1.26 (t, J= 7.1 Hz, 3H).

$$\text{(ES,} m / z ) : {[ \text{M+H]}}^{+} 501.4.$$

### Preparation of Compounds 11B-11J

Compounds 11B-11J as indicated in TABLE 10 were prepared in a similar manner and according to the general synthetic schemes and procedures described herein.

TABLE 10

COMPOUNDS IIB THROUGH IIJ

| Cmpd. No. | Structure | ¹H NMR (300MhZ) | MS (M+H)⁺ |
|---|---|---|---|
| 11B | 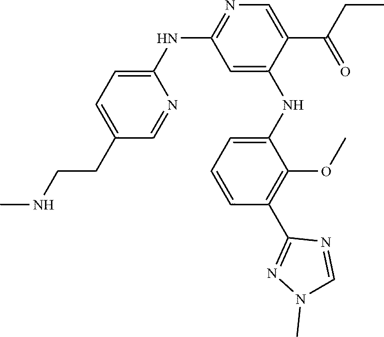 [SMILES: CCC(=O)c1cnc(Nc2ccc(CCNC)cn2)cc1Nc1cccc(-c2ncn(C)n2)c1OC] | (Methanol-d4) δ 8.93 (s, 1H), 8.53 (m, 1H), 8.31 (m, 1H), 7.84 (m, 2H), 7.56 (m, 1H), 7.38 (m, 1H), 7.02 (m, 1H), 6.46 (m, 1H), 4.03 (s, 3H), 3.71 (s, 3H), 3.3-3.25 (m, 2H), 3.15 (m, 2H), 3.03 (m, 2H), 2.75 (m, 3H), 1.26 (m, 3H). | 487.25 |
| 11C | 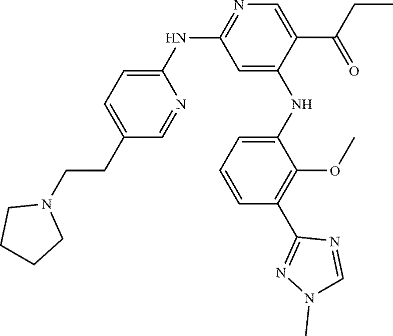 [SMILES: CCC(=O)c1cnc(Nc2ccc(CCN3CCCC3)cn2)cc1Nc1cccc(-c2ncn(C)n2)c1OC] | (Methanol-d4) δ 8.79 (s, 1H), 8.51 (m, 1H), 8.10 (m, 1H), 7.81 (m, 1H), 7.74 (m, 1H), 7.66 (m, 1H), 7.59 (m, 1H), 7.36 (m, 2H), 4.04 (s, 3H), 3.74 (s, 3H), 3.07 (m, 2H), 2.84-2.79 (m, 8H), 1.93 (m, 4H), 1.31-1.2 (m, 3H). | 527.30 |
| 11D | 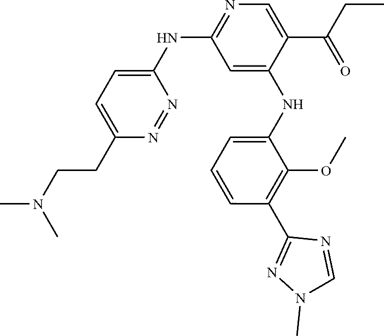 [SMILES: CCC(=O)c1cnc(Nc2ccc(CCN(C)C)nn2)cc1Nc1cccc(-c2ncn(C)n2)c1OC] | (Methanol-d4) δ 9.00 (s, 1H), 8.56 (s, 1H), 7.89 (m, 1H), 7.75 (m, 1H), 7.59 (m, 1H), 7.40 (m, 2H), 6.62 (m, 1H), 4.05 (s, 3H), 3.73-3.69 (m, 5H), 3.45 (m, 2H), 3.20 (m, 2H), 3.00 (s, 6H), 1.30 (m, 3H). | 502.30 |
| 11E | 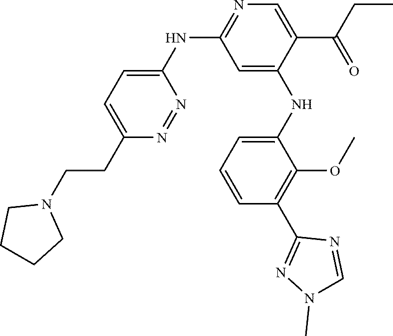 [SMILES: CCC(=O)c1cnc(Nc2ccc(CCN3CCCC3)nn2)cc1Nc1cccc(-c2ncn(C)n2)c1OC] | (Methanol-d4) δ 8.84 (s, 1H), 8.52 (s, 1H), 8.15 (m, 1H), 7.67 (m, 2H), 7.53 (m, 1H), 7.37 (m, 1H), 7.31 (m, 1H), 4.04 (s, 3H), 3.73 (s, 3H), 3.4-3.05 (m, 10H), 2.03 (m, 4H), 1.25 (m, 3H). | 528.25 |
| 11F | 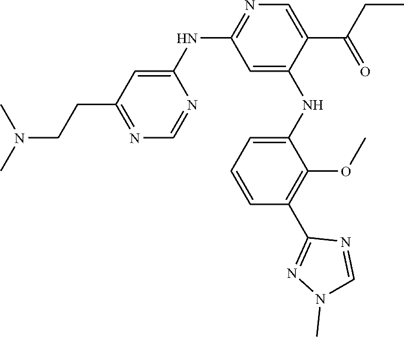 [SMILES: CCC(=O)c1cnc(Nc2cc(CCN(C)C)ncn2)cc1Nc1cccc(-c2ncn(C)n2)c1OC] | (Methanol-d4) δ 8.94 (s, 1H), 8.88 (m, 1H), 8.55 (m, 1H), 7.88 (m, 1H), 7.57 (m, 1H), 7.39 (m, 1H), 6.98 (m, 1H), 6.65 (m, 1H), 4.03 (s, 3H), 3.70 (s, 3H), 3.59 (m, 2H), 3.3-3.13 (m, 4H), 2.97 (s, 6H), 1.27 (m, 3H). | 502.3 |
| 11G | 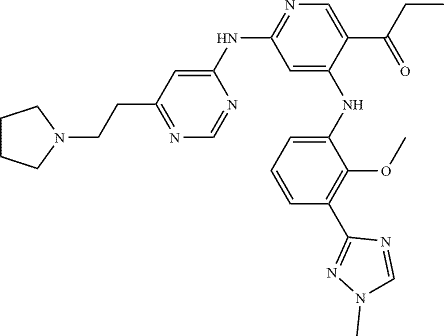 [SMILES: CCC(=O)c1cnc(Nc2cc(CCN3CCCC3)ncn2)cc1Nc1cccc(-c2ncn(C)n2)c1OC] | (DMSO-d6) δ 11.16 (s, 1H), 10.28 (s, 1H), 9.04 (m, 1H), 8.92 (m, 1H), 8.59 (m, 2H), 7.83 (m, 1H), 7.62 (m, 2H), 7.33 (m, 1H), 3.96 (s, 3H), 3.75 (s, 3H), 3.12 (m, 2H), 2.75 (m, 4H), 2.5-2.4 (m, 4H), 1.67 (m, 4H), 1.14 (m, 3H). | 528.30 |
| 11H | 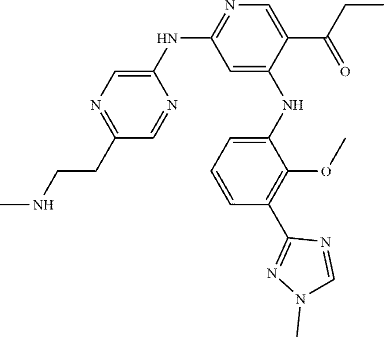 [SMILES: CCC(=O)c1cnc(Nc2cnc(CCNC)cn2)cc1Nc1cccc(-c2ncn(C)n2)c1OC] | (Methanol-d4) δ 8.89 (s, 1H), 8.53 (m, 1H), 8.36 (m, 2H), 7.87 (m, 1H), 7.57 (m, 1H), 7.39 (m, 1H), 6.61 (m, 1H), 4.03 (s, 3H), 3.71 (s, 3H), 3.48 (m, 2H), 3.35-3.3 (m, 2H), 3.14 (m, 2H), 2.78 (m, 3H), 1.28 (m, 3H). | 488.05 |
| 11I | 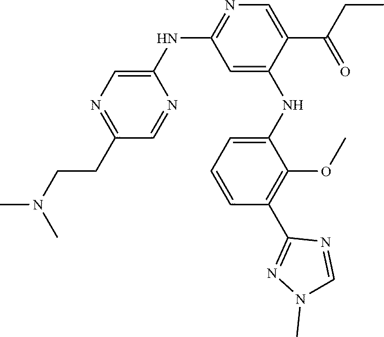 [SMILES: CCC(=O)c1cnc(Nc2cnc(CCN(C)C)cn2)cc1Nc1cccc(-c2ncn(C)n2)c1OC] | (Methanol-d4) δ 8.89 (s, 1H), 8.53 (m, 1H), 8.36 (m, 2H), 7.87 (m, 1H), 7.57 (m, 1H), 7.39 (m, 1H), 6.60 (m, 1H), 4.03 (s, 3H), 3.71 (s, 3H), 3.59 (m, 2H), 3.35-3.3 (m, 2H), 3.16 (m, 2H), 2.98 (s, 6H), 1.27 (m, 3H). | 502.1 |
| 11J | 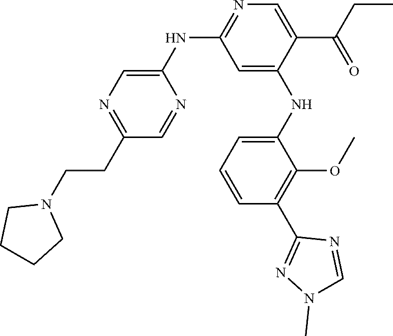 [SMILES: CCC(=O)c1cnc(Nc2cnc(CCN3CCCC3)cn2)cc1Nc1cccc(-c2ncn(C)n2)c1OC] | (Methanol-d4) δ 8.85 (s, 1H), 8.82 (m, 1H), 8.50 (m, 1H), 8.15 (m, 1H), 7.72-7.66 (m, 3H), 7.34 (m, 1H), 4.04 (s, 3H), 3.74 (s, 3H), 3.10 (m, 2H), 3.01 (m, 4H), 2.81 (m, 4H), 1.90 (m, 4H), 1.31-1.25 (m, 3H). | 528.15 |

### Example 12

Preparation of 1-(4-((2-methoxy-3-(1-methyl-1H-1,2,4-triazol-3-yl)phenyl)amino)-6-((5-(methylsulfonyl)pyridin-2-yl)amino)pyridin-3-yl)propan-1-one (Compound 12A)

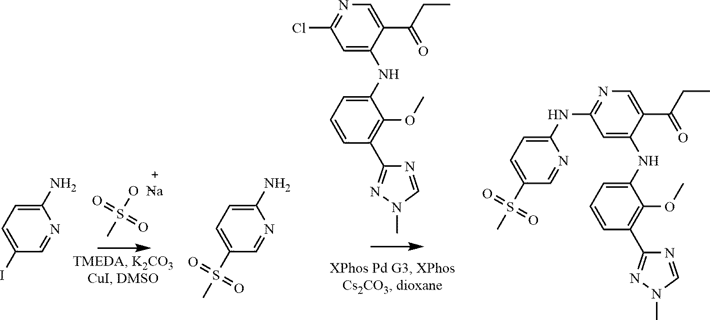

### Step 1: 5-(methylsulfonyl)pyridin-2-amine

5-iodopyridin-2-amine (200 mg, 0.91 mmol), sodium methyl sulfinate (186 mg, 1.82 mmol) and potassium carbonate (125 mg, 0.91 mmol) was suspended in dimethyl sulfoxide (10 mL). Copper(I)iodide (35 mg, 0.18 mmol) and N,N,N′,N′-tetramethylethylenediamine (53 mg, 0.46 mmol) was added to the suspension and stirred at 100° C. under nitrogen overnight. The mixture was cooled, diluted with water and ethyl acetate. The biphasic mixture was passed through a celite bed. The organic layer was separated and washed with water, brine, dried over anhydrous sodium sulfate, and concentrated. The residue was purified with silica gel chromatography eluting with ethyl acetate to yield 5-(methylsulfonyl)pyridin-2-amine (130 mg, 83%) as a solid.

### Step 2: 1-(4-((2-methoxy-3-(1-methyl-1H-1,2,4-triazol-3-yl)phenyl)amino)-6-((5-(methylsulfonyl)pyridin-2-yl)amino)pyridin-3-yl)propan-1-one

1-(6-chloro-4-((2-methoxy-3-(1-methyl- 1H-1,2,4-triazol-3-yl)phenyl)amino)pyridin-3-yl)propan-1-one (100 mg, 0.23 mmol) and 5-methylsulfonylpyridin-2-amine (56 mg, 0.33 mmol) was suspended in 1,4-dioxane (10 mL), added XPhos (52 mg, 0.11 mmol), XPhos Pd G3 (46 mg, 0.05 mmol) and cesium carbonate (265 mg, 0.81 mmol). The mixture was stirred at 100° C. under nitrogen overnight. The reaction mixture was concentrated and purified with silica gel chromatography eluting with dichloromethane:methanol (20:1). The crude product was repurified by Prep-HPLC to yield 1-(4-((2-methoxy-3-(1-methyl-1H-1,2,4-triazol-3-yl)phenyl)amino)-6-((5-(methylsulfonyl)pyridin-2-yl)amino)pyridin-3-yl)propan-1-one (102 mg, 74%) as a solid. ¹H NMR (300 MHz, DMSO-d6) δ 11.09 (s, 1H), 10.56 (s, 1H), 8.91 (s, 1H), 8.65 - 8.54 (m, 2H), 8.12 (dd, J = 8.9, 2.6 Hz, 1H), 7.92 - 7.80 (m, 2H), 7.66 (dq, J = 8.0, 1.7 Hz, 2H), 7.34 (t, J = 7.9 Hz, 1H), 3.96 (s, 3H), 3.74 (s, 3H), 3.23 (s, 3H), 3.12 (q, J = 7.2 Hz, 2H), 1.14 (t, J = 7.2 Hz, 3H)

$$\text{LC-MS (ES, m/z): [M+H]+ 508} \text{.10}$$

### Example 13

Preparation of 1-(4-((2-methoxy-3-(1-methyl-1H-1,2,4-triazol-3-yl)phenyl)amino)-6-((5-( (methylsulfonyl)methyl)pyridin-2-yl )amino )pyridin-3-yl )propan-1-one (Compound 13A)

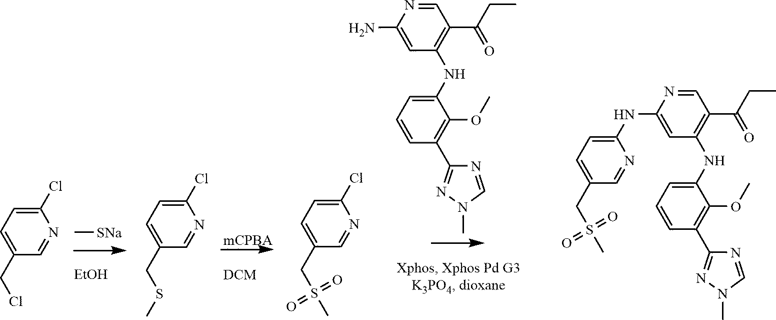

### Step 1: 2-chloro-5-((methylthio)methyl)pyridine

A solution of 2-chloro-5-(chloromethyl)pyridine (500 mg, 3.1 mmol) in ethanol (10 mL) was added to a suspension of sodium methanethiolate (259 mg, 3.7 mmol) in ethanol (20 ml) and the mixture was stirred at room temperature overnight. The solvent was removed under reduced pressure and the residue was redissolved in diethyl ether:EtOAc (1:1) and mixed with brine. The two phases were separated and the organic layer was dried over anhydrous sodium sulfate, filtered, concentrated and purified by eluting through a silica gel plug with 40% ethyl acetate in hexane to yield 2-chloro-5-((methylthio)methyl)pyridine (457 mg, 85%) as an oil.

### Step 2: 2-chloro-5-((methylsulfonyl)methyl)pyridine

3-chlorobenzenecarboperoxoic acid (77%, 653 mg, 2.91 mmol) was added to a solution of 2-chloro-5-((methylthio)methyl)pyridine (220 mg, 1.27 mmol) in dichloromethane (9 mL) at 0° C. The cooling bath was removed and the solution was stirred at room temperature overnight. The mixture was diluted with dichloromethane, washed with 10% aqueous potassium carbonate solution, dried with magnesium sulfate, and concentrated. The residue was chromatographed on prep-TLC (cyclohexane:ethyl acetate; 3:7) to yield 2-chloro-5-((methylsulfonyl)methyl)pyridine (199 mg, 76%) as a solid.

### Step 3: 1-(4-((2-methoxy-3-(1-methyl-1H-1,2,4-triazol-3-yl)phenyl)amino)-6-((5-((methylsulfonyl)methyl)pyridin-2-yl)amino)pyridin-3-yl)propan-1-one

To a solution of 1-(6-amino-4-((2-methoxy-3-(1-methyl-1H-1,2,4-triazol-3-yl)phenyl)amino)pyridin-3-yl)propan-1-one (100 mg, 0.28 mmol) in 1,4-dioxane (10 mL) was added 2-chloro-5-((methylsulfonyl)methyl)pyridine (88 mg, 0.43 mmol), Xphos (27 mg, 0.05 mmol) Xphos Pd G3 (24 mg, 0.03 mmol) and potassium phosphate (180 mg, 0.85 mmol). The reaction mixture was stirred at 100° C. under N2 overnight. After cooling to room temperature, the mixture was filtered and concentrated. The residue was purified by prep HPLC to yield 1-(4-((2-methoxy-3-(1-methyl-1H-1,2,4-triazol-3-yl)phenyl)amino)-6-((5-((methylsulfonyl)methyl)pyridin-2-yl)amino)pyridin-3-yl)propan-1-one (56 mg, 38%) as a solid. ¹H NMR (300 MHz, DMSO-d6) δ 11.12 (s, 1H), 10.10 (s, 1H), 8.88 (s, 1H), 8.58 (s, 1H), 8.20 (d, J = 2.3 Hz, 1H), 8.00 (s, 1H), 7.76 - 7.59 (m, 4H), 7.32 (t, J = 7.9 Hz, 1H), 4.45 (s, 2H), 3.97 (s, 3H), 3.77 (s, 3H), 3.11 (q, J= 7.2 Hz, 2H), 2.94 (s, 3H), 1.15 (t, J = 7.2 Hz, 3H).

$$\text{LC-MS} \left(\text{ES,} \text{m} / \text{z}\right) : \left[\text{M+H}\right] + = 522.2$$

### Preparation of Compounds 13B-13AA

Compounds 13B-13AA, as indicated in TABLE 11, were prepared in a similar manner and according to the general synthetic schemes and procedures described herein.

TABLE 11

COMPOUNDS 13B THROUGH 13AA

| Cmpd. No. | Structure | ¹H NMR | MS (M+H)⁺ |
|---|---|---|---|
| 13B | 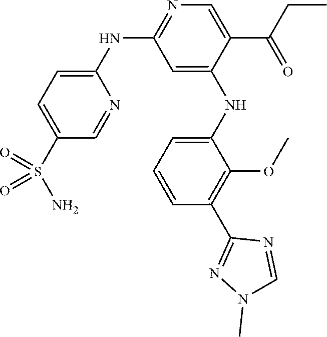 [SMILES: CCC(=O)c1cnc(Nc2ccc(S(N)(=O)=O)cn2)cc1Nc1cccc(-c2ncn(C)n2)c1OC] | 1H NMR (300 MHz, DMSO-d6) δ 11.12 (s, 1H), 10.8 (br s, 1H), 8.92 (s, 1H), 8.59 (m, 2H), 8.08 (m, 1H), 7.75-7.35 (m, 6H), 7.1-6.9 (m, 1H), 4.04 (s, 3H), 3.75 (s, 3H), 3.13 (m, 2H), 1.14 (m, 3H). | 509.05 |
| 13C | 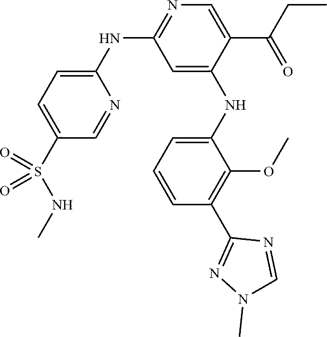 [SMILES: CCC(=O)c1cnc(Nc2ccc(S(=O)(=O)NC)cn2)cc1Nc1cccc(-c2ncn(C)n2)c1OC] | 1H NMR (300 MHz, DMSO-d6) δ 11.12 (s, 1H), 10.9 (br s, 1H), 8.92 (s, 1H), 8.58 (m, 2H), 8.06 (m, 1H), 7.76 (m, 1H), 7.70-7.50 (m, 3H), 7.35 (m, 1H), 3.96 (s, 3H), 3.75 (s, 3H), 3.16 (m, 2H), 2.45 (m, 3H), 1.15 (m, 3H). | 523.15 |
| 13D | 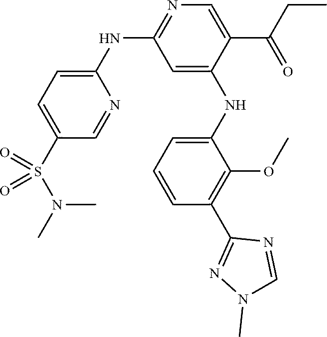 [SMILES: CCC(=O)c1cnc(Nc2ccc(S(=O)(=O)N(C)C)cn2)cc1Nc1cccc(-c2ncn(C)n2)c1OC] | 1H NMR (300 MHz, DMSO-d6) δ 11.10 (s, 1H), 10.92 (br s, 1H), 8.90 (s, 1H), 8.57 (m, 1H), 8.48 (m, 1H), 8.04 (m, 1H), 7.72-7.60 (m, 3H), 7.45-7.30 (m, 2H), 3.94 (s, 3H), 3.72 (s, 3H), 3.14 (m, 2H), 2.61 (s, 6H), 1.14 (m, 3H). | 537.15 |
| 13E | 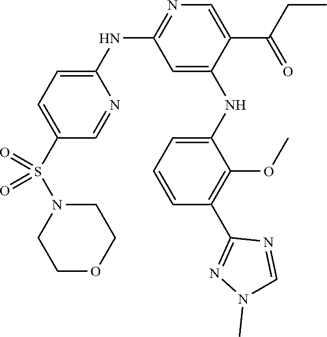 [SMILES: CCC(=O)c1cnc(Nc2ccc(S(=O)(=O)N3CCOCC3)cn2)cc1Nc1cccc(-c2ncn(C)n2)c1OC] | 1H NMR (400 MHz, DMSO-d6) δ 11.23 (s, 1H), 11.15 (s, 1H), 8.93 (s, 1H), 8.60 (m, 1H), 8.51 (m, 1H), 8.07 (m, 1H), 7.78 (m, 1H), 7.64 (m, 1H), 7.52 (m, 1H), 7.37 (m, 1H), 7.22 (m, 1H), 3.96 (s, 3H), 3.74 (s, 3H), 3.63 (m, 4H), 3.14 (m, 2H), 2.92 (m, 4H), 1.15 (m, 3H). | 579.20 |
| 13F | 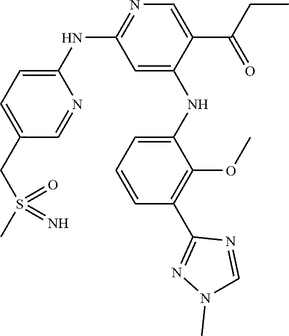 [SMILES: CCC(=O)c1cnc(Nc2ccc(CS(C)(=N)=O)cn2)cc1Nc1cccc(-c2ncn(C)n2)c1OC] | 1H NMR (300 MHz, DMSO-d6) δ 11.13 (s, 1H), 10.07 (s, 1H), 8.88 (s, 1H), 8.58 (m, 1H), 8.21 (m, 1H), 8.15 (s, 1H), 8.02 (m, 1H), 7.68-7.57 (m, 4H), 7.32 (m, 1H), 4.32 (m, 2H), 3.97 (s, 3H), 3.77 (s, 3H), 3.12 (m, 2H), 2.80 (s, 3H), 1.16 (m, 3H). | 521.10 |
| 13G | 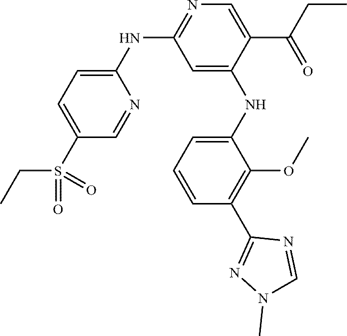 [SMILES: CCC(=O)c1cnc(Nc2ccc(S(=O)(=O)CC)cn2)cc1Nc1cccc(-c2ncn(C)n2)c1OC] | 1H NMR (300 MHz, DMSO-d6) δ 11.10 (s, 1H), 10.60 (s, 1H), 8.93 (s, 1H), 8.58 (m, 2H), 8.11 (m, 1H), 7.87 (m, 2H), 7.68 (m, 2H), 7.35 (m, 1H), 3.97 (s, 3H), 3.76 (s, 3H), 3.34 (m, 2H), 3.15 (m, 2H), 1.15 (m, 6H). | 522.15 |
| 13H | 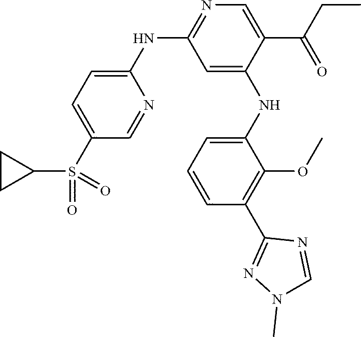 [SMILES: CCC(=O)c1cnc(Nc2ccc(S(=O)(=O)C3CC3)cn2)cc1Nc1cccc(-c2ncn(C)n2)c1OC] | 1H NMR (300 MHz, DMSO-d6) δ 11.10 (s, 1H), 10.58 (s, 1H), 8.92 (s, 1H), 8.59 (m, 2H), 8.13 (m, 1H), 7.87 (m, 2H), 7.68 (m, 2H), 7.35 (m, 1H), 3.97 (s, 3H), 3.76 (s, 3H), 3.15 (m, 2H), 2.90 (m, 1H), 1.2-1.0 (m, 7H). | 534.10 |
| 13I | 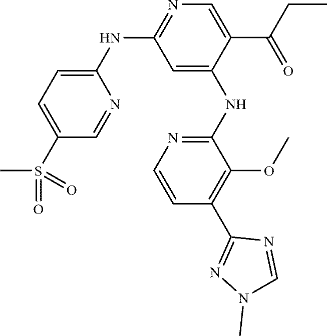 [SMILES: CCC(=O)c1cnc(Nc2ccc(S(C)(=O)=O)cn2)cc1Nc1nccc(-c2ncn(C)n2)c1OC] | 1H NMR (400 MHz, DMSO-d6) δ 12.39 (s, 1H), 10.75 (s, 1H), 9.60 (s, 1H), 9.00 (s, 1H), 8.78 (m, 1H), 8.68 (m, 1H), 8.26 (m, 1H), 8.16 (m, 1H), 7.95 (m, 1H), 7.53 (m, 1H), 4.01 (s, 3H), 3.93 (s, 3H), 3.32 (s, 3H), 3.16 (m, 2H), 1.16 (m, 3H). | 509.30 |
| 13J | 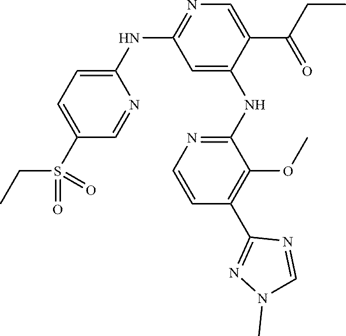 [SMILES: CCC(=O)c1cnc(Nc2ccc(S(=O)(=O)CC)cn2)cc1Nc1nccc(-c2ncn(C)n2)c1OC] | 1H NMR (400 MHz, DMSO-d6) δ 12.39 (s, 1H), 10.77 (s, 1H), 9.59 (s, 1H), 9.00 (s, 1H), 8.72 (m, 1H), 8.68 (m, 1H), 8.24 (m, 1H), 8.13 (m, 1H), 7.97 (m, 1H), 7.52 (m, 1H), 4.01 (s, 3H), 3.93 (s, 3H), 3.35 (m, 2H), 3.15 (m, 2H), 1.16 (m, 6H). | 523.30 |
| 13K | 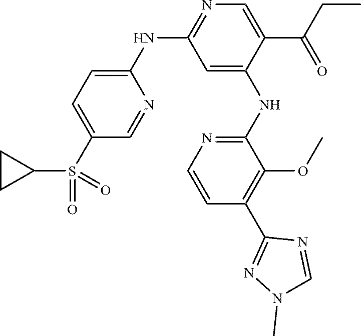 [SMILES: CCC(=O)c1cnc(Nc2ccc(S(=O)(=O)C3CC3)cn2)cc1Nc1nccc(-c2ncn(C)n2)c1OC] | 1H NMR (400 MHz, DMSO-d6) δ 12.37 (s, 1H), 10.74 (s, 1H), 9.55 (s, 1H), 8.98 (s, 1H), 8.71 (m, 1H), 8.66 (m, 1H), 8.22 (m, 1H), 8.14 (m, 1H), 7.94 (m, 1H), 7.50 (m, 1H), 3.99 (s, 3H), 3.91 (s, 3H), 3.15 (m, 2H), 2.92 (m, 1H), 1.21-1.03 (m, 7H). | 535.15 |
| 13L | 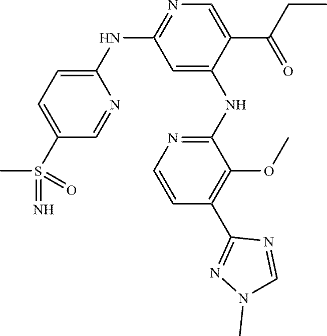 [SMILES: CCC(=O)c1cnc(Nc2ccc(S(C)(=N)=O)cn2)cc1Nc1nccc(-c2ncn(C)n2)c1OC] | 1H NMR (400 MHz, Methanol-d4) δ 8.82 (m, 1H), 8.64 (m, 1H), 8.48 (m, 1H), 8.15 (m, 1H), 7.77 (m, 1H), 7.43 (m, 1H), 7.25 (m, 2H), 4.01 (s, 3H), 3.98 (s, 3H), 3.51 (s, 3H), 2.95 (m, 2H), 1.17 (m, 3H). | 508.15 |
| 13M | 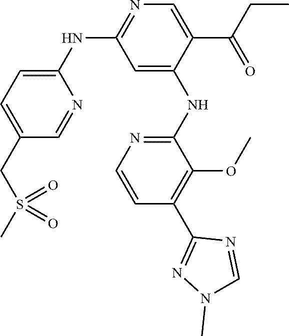 [SMILES: CCC(=O)c1cnc(Nc2ccc(CS(C)(=O)=O)cn2)cc1Nc1nccc(-c2ncn(C)n2)c1OC] | 1H NMR (400 MHz, DMSO-d6) δ 12.39 (s, 1H), 10.28 (s, 1H), 9.55 (s, 1H), 8.95 (s, 1H), 8.69 (m, 1H), 8.35 (m, 1H), 8.21 (m, 1H), 7.75 (m, 2H), 7.52 (m, 1H), 4.49 (s, 2H), 4.02 (s, 3H), 3.94 (s, 3H), 3.13 (m, 2H), 2.97 (s, 3H), 1.17 (m, 3H). | 523.20 |
| 13N | 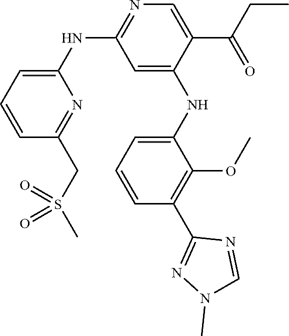 [SMILES: CCC(=O)c1cnc(Nc2cccc(CS(C)(=O)=O)n2)cc1Nc1cccc(-c2ncn(C)n2)c1OC] | 1H NMR (300 MHz, DMSO-d6) δ 11.09 (s, 1H), 10.09 (s, 1H), 8.87 (s, 1H), 8.58 (s, 1H), 8.05 (m, 1H), 7.75-7.67 (m, 3H), 7.52 (m, 1H), 7.36 (m, 1H), 7.02 (m, 1H), 4.35 (s, 2H), 3.96 (s, 3H), 3.72 (s, 3H), 3.12 (m, 2H), 2.73 (s, 3H), 1.14 (m, 3H). | 522.35 |
| 130 | 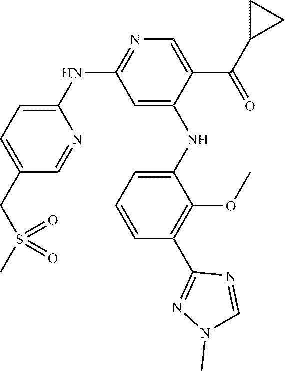 [SMILES: COc1c(Nc2cc(Nc3ccc(CS(C)(=O)=O)cn3)ncc2C(=O)C2CC2)cccc1-c1ncn(C)n1] | 1H NMR (300 MHz, DMSO-d6) δ 11.09 (s, 1H), 10.14 (s, 1H), 9.12 (s, 1H), 8.57 (s, 1H), 8.19 (m, 1H), 8.01 (m, 1H), 7.75-7.60 (m, 4H), 7.30 (m, 1H), 4.44 (s, 2H), 3.95 (s, 3H), 3.72 (s, 3H), 3.02-2.93 (m, 4H), 1.11-1.04 (m, 4H). | 534.30 |
| 13P | 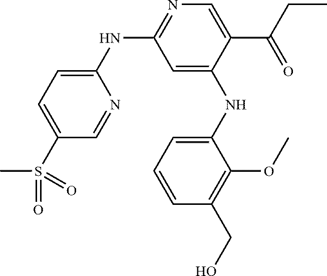 [SMILES: CCC(=O)c1cnc(Nc2ccc(S(C)(=O)=O)cn2)cc1Nc1cccc(CO)c1OC] | 1H NMR (400 MHz, Methanol-d4) δ 8.84 (m, 1H), 8.62 (m, 1H), 8.09 (m, 1H), 7.81 (m, 1H), 7.67 (m, 1H), 7.52 (m, 1H), 7.31 (m, 1H), 7.25 (m, 1H), 4.74 (s, 2H), 3.77 (s, 3H), 3.12 (m, 5H), 1.27 (m, 3H). | 457.10 |
| 13Q | 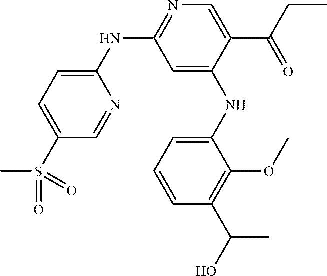 [SMILES: CCC(=O)c1cnc(Nc2ccc(S(C)(=O)=O)cn2)cc1Nc1cccc(C(C)O)c1OC] | 1H NMR (400 MHz, Methanol-d4) δ 8.83 (m, 1H), 8.59 (m, 1H), 8.07 (m, 1H), 7.81 (m, 1H), 7.63 (m, 1H), 7.47 (m, 1H), 7.39 (m, 1H), 7.26 (m, 1H), 5.22 (m, 1H), 3.74 (s, 3H), 3.15-3.05 (m, 5H), 1.46 (m, 3H), 1.24 (m, 3H). | 471.10 |
| 13R | 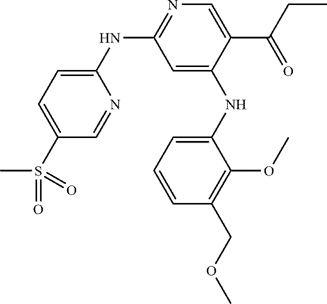 [SMILES: CCC(=O)c1cnc(Nc2ccc(S(C)(=O)=O)cn2)cc1Nc1cccc(COC)c1OC] | 1H NMR (300 MHz, DMSO-d6) δ 10.98 (s, 1H), 10.55 (s, 1H), 8.90 (s, 1H), 8.59 (s, 1H), 8.13 (m, 1H), 7.82 (m, 2H), 7.55 (m, 1H), 7.25 (m, 2H), 4.49 (s, 2H), 3.69 (s, 3H), 3.35 (s, 3H), 3.23 (s, 3H), 3.12 (m, 2H), 1.13 (m, 3H). | 471.20 |
| 13D | 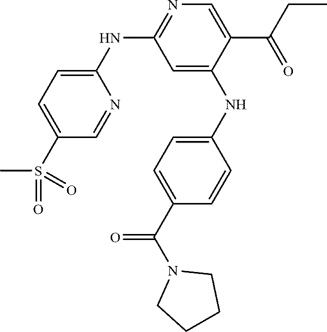 [SMILES: CCC(=O)c1cnc(Nc2ccc(S(C)(=O)=O)cn2)cc1Nc1ccc(C(=O)N2CCCC2)cc1] | 1H NMR (300 MHz, DMSO-d6) δ 10.98 (s, 1H), 10.68 (s, 1H), 8.92 (s, 1H), 8.61 (s, 1H), 8.16 (m, 1H), 7.86-7.65 (m, 4H), 7.43 (m, 2H), 3.4 (s, 3H), 3.25 (m, 4H), 3.15 (m, 2H), 1.89 (m, 4H), 1.16 (m, 3H). | 492.10 |
| 13T | 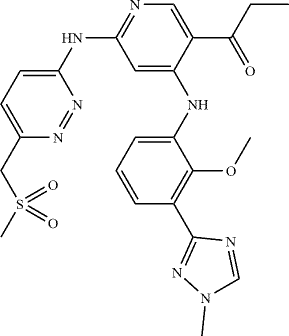 [SMILES: CCC(=O)c1cnc(Nc2ccc(CS(C)(=O)=O)nn2)cc1Nc1cccc(-c2ncn(C)n2)c1OC] | 1H NMR (300 MHz, DMSO-d6) δ 11.08 (s, 1H), 10.46 (s, 1H), 8.89 (s, 1H), 8.57 (s, 1H), 8.18 (m, 1H), 7.64 (m, 4H), 7.27 (m, 1H), 4.76 (s, 2H), 3.96 (s, 3H), 3.75 (s, 3H), 3.14 (m, 2H), 3.03 (s, 3H), 1.16 (m, 3H). | 523.20 |
| 13U | | 1H NMR (300 MHz, DMSO-d6) δ 11.09 (s, 1H), 10.42 (s, 1H), 8.98 (s, 1H), 8.91 (m, 1H), 8.57 (m, 1H), 8.27 (m, 1H), 7.78 (m, 1H), 7.66 (m, 2H), 7.31 (m, 1H), 4.60 (s, 2H), 3.95 (s, 3H), 3.74 (s, 3H), 3.13 (m, 2H), 3.01 (s, 3H), 1.14 (m, 3H). | 523.15 |
| 13V | 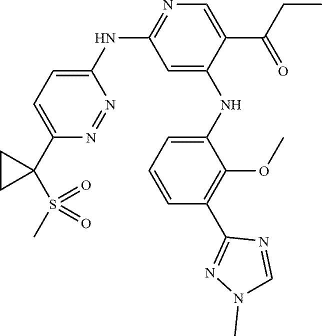 [SMILES: CCC(=O)c1cnc(Nc2ccc(C3(S(C)(=O)=O)CC3)nn2)cc1Nc1cccc(-c2ncn(C)n2)c1OC] | 1H NMR (300 MHz, DMSO-d6) δ 11.07 (s, 1H), 10.49 (s, 1H), 8.87 (s, 1H), 8.56 (m, 1H), 8.18 (m, 1H), 7.73 (m, 1H), 7.62 (m, 3H), 7.25 (m, 1H), 3.94 (s, 3H), 3.74 (s, 3H), 3.09 (m, 2H), 3.01 (s, 3H), 1.71 (m, 2H), 1.47 (m, 2H), 1.14 (m, 3H). | 549.20 |
| 13W | 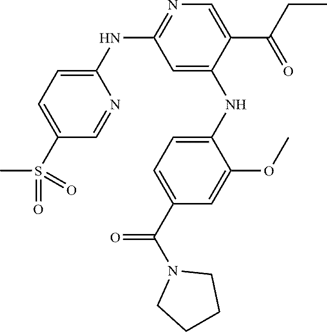 [SMILES: CCC(=O)c1cnc(Nc2ccc(S(C)(=O)=O)cn2)cc1Nc1ccc(C(=O)N2CCCC2)cc1OC] | 1H NMR (400 MHz, DMSO-d6) δ 10.93 (s, 1H), 10.56 (s, 1H), 8.89 (s, 1H), 8.59 (s, 1H), 8.13 (m, 1H), 7.95 (m, 1H), 7.78 (m, 1H), 7.59 (m, 1H), 7.26 (m, 2H), 3.90 (s, 3H), 3.50 (m, 4H), 3.25 (s, 3H), 3.11 (m, 2H), 1.88 (m, 4H), 1.12 (m, 3H). | 524.15 |
| 13X | 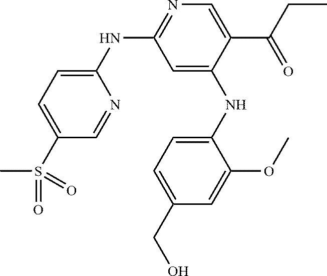 [SMILES: CCC(=O)c1cnc(Nc2ccc(S(C)(=O)=O)cn2)cc1Nc1ccc(CO)cc1OC] | 1H NMR (400 MHz, DMSO-d6) δ 10.82 (s, 1H), 10.51 (s, 1H), 8.87 (s, 1H), 8.59 (s, 1H), 8.12 (m, 1H), 7.84 (m, 1H), 7.73 (m, 1H), 7.47 (m, 1H), 7.13 (m, 1H), 7.01 (m, 1H), 5.27 (m, 1H), 4.54 (m, 2H), 3.86 (s, 3H), 3.24 (s, 3H), 3.11 (m, 2H), 1.13 (m, 3H). | 457.1 |
| 13Y | 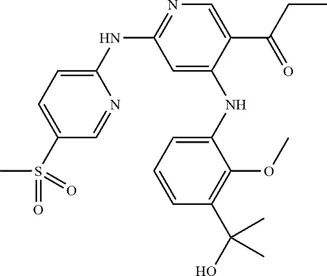 [SMILES: CCC(=O)c1cnc(Nc2ccc(S(C)(=O)=O)cn2)cc1Nc1cccc(C(C)(C)O)c1OC] | 1H NMR (400 MHz, Methanol-d4) δ 8.86 (m, 1H), 8.61 (m, 1H), 8.26 (m, 1H), 8.09 (m, 1H), 7.63 (m, 1H), 7.54 (m, 1H), 7.42 (m, 1H), 7.22 (m, 1H), 3.75 (s, 3H), 3.14 (m, 5H), 1.66 (s, 6H), 1.31 (m, 3H). | 485.2 |
| 13Z | 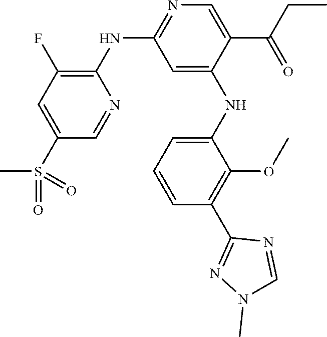 [SMILES: CCC(=O)c1cnc(Nc2ncc(S(C)(=O)=O)cc2F)cc1Nc1cccc(-c2ncn(C)n2)c1OC] | 1H NMR (400 MHz, DMSO-d6) δ 11.11 (s, 1H), 10.04 (s, 1H), 8.90 (m, 1H), 8.57 (m, 1H), 8.46 (m, 1H), 8.12 (m, 2H), 7.68 (m, 2H), 7.37 (m, 1H), 3.96 (s, 3H), 3.75 (s, 3H), 3.31 (s, 3H), 3.15 (m, 2H), 1.15 (m, 3H). | 526.15 |
| 13 AA | 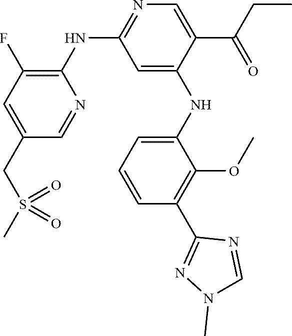 [SMILES: CCC(=O)c1cnc(Nc2ncc(CS(C)(=O)=O)cc2F)cc1Nc1cccc(-c2ncn(C)n2)c1OC] | 1H NMR (300 MHz, DMSO-d6) δ 11.10 (s, 1H), 9.53 (s, 1H), 8.86 (m, 1H), 8.57 (m, 1H), 8.09 (m, 1H), 8.00 (m, 1H), 7.72-7.63 (m, 3H), 7.31 (m, 1H), 4.52 (s, 2H), 3.96 (s, 3H), 3.75 (s, 3H), 3.12 (m, 2H), 2.96 (s, 3H), 1.14 (m, 3H). | 540.25 |

### Example 14

Preparation of imino(6-((4-((2-methoxy-3-(1-methyl-1H-1,2,4-triazol-3-yl)phenyl)amino)-5-propionylpyridin-2-yl)amino)pyridin-3-yl)(methyl)-16-sulfanone (Compound 14A)

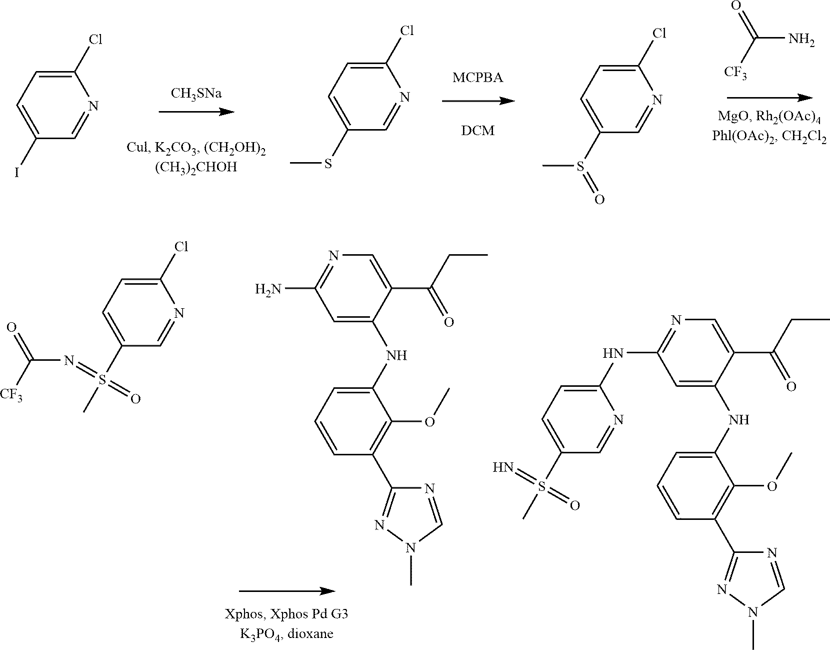

### Step 1: 2-chloro-5-(methylsulfinyl)pyridine

Into a bottom flask maintained with an inert atmosphere of nitrogen was placed 2-chloro-5-(methylthio)pyridine (536 mg, 3.36 mmol) in dichloromethane (50 ml), cooled in an ice bath, then added 3-chloroperbenzoic acid (637 mg, 3.69 mmol). The mixture was stirred for 30 minutes at 0° C. The mixture was basified to pH 7 with ammonia in methanol, filtered off solids, then concentrated filtrates. The residue was purified by prep-TLC (dichloromethane:methanol; 20:1) to yield 2-chloro-5-methylsulfinyl-pyridine (321 mg, 54%) as a solid.

### Step 2: N-((6-chloropyridin-3-yl)(methyl)(oxo)-16-sulfaneylidene)-2,2,2-trifluoroacetamide

Into a 40 mL tube maintained with an inert atmosphere of nitrogen was placed 2-chloro-5-methylsulfinyl-pyridine (150 mg, 0.85 mmol), 2,2,2-trifluoroacetamide (193 mg, 1.71 mmol), magnesium oxide (138 mg, 3.42 mmol), dirhodium tetraacetate (11 mg, 0.03 mmol), and iodobenzene diacetate (413 mg, 1.28 mmol) in dichloromethane (15 mL). The resulting mixture was stirred at room temperature overnight. Concentrated under vacuum and purified by prep-TLC (petroleum ether:ethyl acetate 1:1) to yield N-((6-chloropyridin-3-yl)(methyl)(oxo)-16-sulfaneylidene)-2,2,2-trifluoroacetamide (224 mg, 92%) as a solid.

### Step 3: imino(6-((4-((2-methoxy-3-(1-methyl-1H-1,2,4-triazol-3-yl)phenyl)amino)-5-propionylpyridin-2-yl)amino)pyridin-3-yl)(methyl)-16-sulfanone

Into a 20 mL tube maintained with an inert atmosphere of nitrogen was placed N-((6-chloropyridin-3-yl)(methyl)(oxo)-16-sulfaneylidene)-2,2,2-trifluoroacetamide (100 mg, 0.35 mmol), 1-(6-amino-4-((2-methoxy-3-(1-methyl-1H-1,2,4-triazol-3-yl)phenyl)amino)pyridin-3-yl)propan-1-one (123 mg, 0.35 mmol), tripotassium phosphate (148 mg, 0.69 mmol), Xphos (33 mg, 0.07 mmol), Xphos Pd G3 (30 mg, 0.035 mmol) in 1,4-dioxane (5 mL). The mixture was stirred at 90° C. for 2 hours. Concentrated the mixture and purified by Prep-HPLC to yield imino(6-((4-((2-methoxy-3-(1-methyl-1H-1,2,4-triazol-3-yl)phenyl)amino)-5-propionylpyridin-2-yl)amino)pyridin-3-yl)(methyl)-16-sulfanone (9.3 mg, 5%) as a solid. ¹H NMR (DMSO-d6, 300 MHz, ppm): 11.11 (s, 1H), 10.52 (s, 1H), 8.92 (s, 1H), 8.67 - 8.55 (m, 2H), 8.12 (dd, J = 8.9, 2.6 Hz, 1H), 7.90 (s, 1H), 7.80 (d, J = 8.9 Hz, 1H), 7.68 (dq, J = 8.1, 1.7 Hz, 2H), 7.34 (t, J = 7.9 Hz, 1H), 3.97 (s, 3H), 3.76 (s, 3H), 3.13 - 3.07 (m, 5H), 1.16 (t, J = 7.2 Hz, 3H).

$$\text{LC-MS} \text{: (ES, m/z): [M+H]+ 507} \text{.25} \text{.}$$

### Example 15

Preparation of 6′-((4-((2-methoxy-3-(1-methyl-1H-1,2,4-triazol-3-yl)phenyl)amino)-5-propionylpyridin-2-yl)amino)-2H-[1,3′-bipyridin]-2-one (Compound 15A)

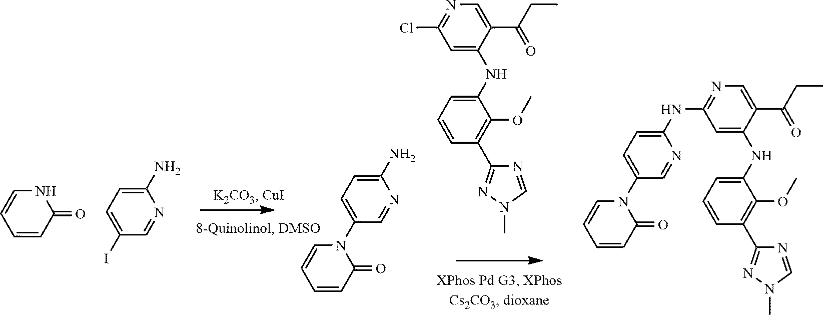

### Step 1: 6′-amino-2H-[1,3′-bipyridin]-2-one

Into a 40 mL tube maintained with an inert atmosphere of nitrogen was placed 5-iodopyridin-2-amine (500 mg, 2.27 mmol), 1H-pyridin-2-one (216 mg, 2.27 mmol), 8-quinolinol (67 mg, 0.45 mmol), potassium carbonate (942 mg, 6.82 mmol), copper(I) iodide (130 mg,0.68 mmol) in dimethyl sulfoxide (25 mL). The resulting mixture was heated to 150° C. overnight. After cooling the mixture was diluted with water and extracted with ethyl acetate (3 × 10 mL). The residue was purified by column chromatography to afford 6′-amino-2H-[1,3′-bipyridin]-2-one (220 mg, 52% yield) as a solid.

### Step 2: 6′-((4-((2-methoxy-3-(1-methyl-1H-1,2,4-triazol-3-yl)phenyl)amino)-5-propionylpyridin-2-yl)amino)-2H-[1,3′-bipyridin]-2-one

Into a 40 mL tube maintained with an inert atmosphere of nitrogen was placed 6′-amino-2H-[1,3′-bipyridin]-2-one (197 mg, 1.05 mmol), 1-(6-chloro-4-((2-methoxy-3-(1-methyl- 1H-1,2,4-triazol-3-yl)phenyl)amino)pyridin-3-yl)propan-1-one (300 mg, 0.81 mmol), XPhos (77 mg, 0.16 mmol), XPhos Pd G3 (69 mg, 0.08 mmol), cesium carbonate (528 mg, 1.62 mmol) and 1,4-dioxane (15 mL) at room temperature. The mixture was at 100° C. for 2 hours. The mixture was concentrated under vacuum and purified by Prep-HPLC to yield 6′-((4-((2-methoxy-3-(1-methyl-1H-1,2,4-triazol-3-yl)phenyl)amino)-5-propionylpyridin-2-yl)amino)-2H-[1,3′-bipyridin]-2-one (119 mg, 28%) as a white solid. ¹H-NMR (DMSO-d6, 300 MHz, ppm): 11.12 (s, 1H), 10.22 (s, 1H), 8.88 (s, 1H), 8.56 (s, 1H), 8.22 (d, J=2.5 Hz, 1H), 7.93 (s, 1H), 7.69 (dddd, J=25.4, 21.3, 8.3, 2.0 Hz, 5H), 7.52 (ddd, J=8.9, 6.6, 2.1 Hz, 1H), 7.33 (t, J=7.9 Hz, 1H), 6.54-6.44 (m, 1H), 6.33 (td, J=6.7, 1.3 Hz, 1H), 3.95 (s, 3H), 3.75 (s, 3H), 3.10 (q, J=7.2 Hz, 2H), 1.14 (t, J=7.2 Hz, 3H).

$$\text{LC-MS (ES, m/z): [M+H]+ 523} \text{.25} \text{.}$$

### Preparation of Compounds 15B-15DD

Compounds 15B-15AA, as indicated in TABLE 12, were prepared in a similar manner and according to the general synthetic schemes and procedures described herein.

TABLE 12

COMPOUNDS 15B THROUGH 15DD

| Cmpd. No. | Structure | ¹H NMR | MS (M+H)⁺ |
|---|---|---|---|
| 15B | 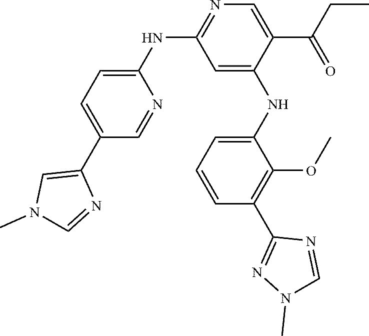 [SMILES: CCC(=O)c1cnc(Nc2ccc(-c3cn(C)cn3)cn2)cc1Nc1cccc(-c2ncn(C)n2)c1OC] | 1H NMR (300 MHz, Methanol-d4) δ 8.97-8.94 (m, 2H), 8.77 (m, 1H), 8.56 (m, 1H), 8.19 (m, 1H), 7.98 (m, 1H), 7.88 (m, 1H), 7.62 (m, 1H), 7.41 (m, 1H), 7.16 (m, 1H), 6.60 (m, 1H), 4.05 (s, 3H), 4.01 (s, 3H), 3.74 (s, 3H), 3.16 (m, 2H), 1.29 (m, 3H). | 510.15 |
| 15C | 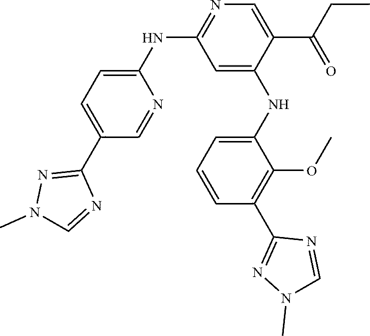 [SMILES: CCC(=O)c1cnc(Nc2ccc(-c3ncn(C)n3)cn2)cc1Nc1cccc(-c2ncn(C)n2)c1OC] | 1H NMR (300 MHz, DMSO-d6) δ 11.11 (s, 1H), 10.21 (s, 1H), 8.89 (s, 1H), 8.75 (m, 1H), 8.55 (m, 2H), 8.18 (m, 1H), 7.97 (m, 1H), 7.73-7.64 (m, 3H), 7.33 (m, 1H), 3.96 (s, 3H), 3.92 (s, 3H), 3.76 (s, 3H), 3.10 (m, 2H), 1.14 (m, 3H). | 511.30 |
| 15D | 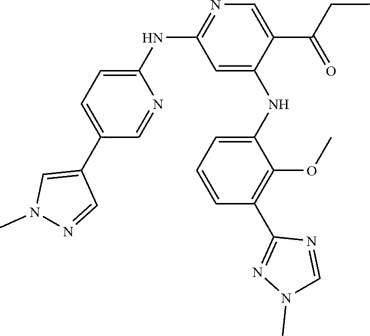 [SMILES: CCC(=O)c1cnc(Nc2ccc(-c3cnn(C)c3)cn2)cc1Nc1cccc(-c2ncn(C)n2)c1OC] | 1H NMR (300 MHz, DMSO-d6) δ 11.23 (s, 1H), 11.08 (s, 1H), 8.96 (s, 1H), 8.58 (m, 1H), 8.50 (m, 1H), 8.23 (m, 1H), 8.04 (m, 1H), 7.93 (m, 1H), 7.83 (m, 1H), 7.56 (m, 1H), 7.36 (m, 1H), 7.10 (m, 1H), 6.62 (m, 1H), 3.94 (s, 3H), 3.87 (s, 3H), 3.73 (s, 3H), 3.13 (m, 2H), 1.14 (m, 3H). | 510.15 |
| 15E | 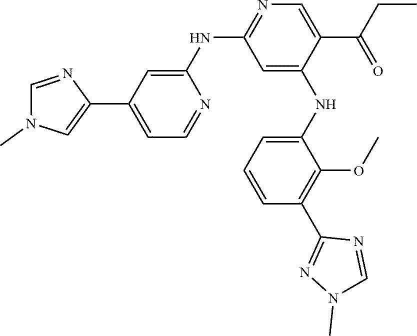 [SMILES: CCC(=O)c1cnc(Nc2cc(-c3cn(C)cn3)ccn2)cc1Nc1cccc(-c2ncn(C)n2)c1OC] | 1H NMR (400 MHz, Methanol-d4) δ 8.96 (m, 1H), 8.60 (m, 1H), 8.42 (m, 2H), 8.10 (m, 1H), 7.88 (m, 1H), 7.62 (m, 1H), 7.52 (m, 1H), 7.41 (m, 1H), 7.35 (m, 1H), 6.54 (m, 1H), 4.06 (s, 3H), 3.93 (m, 3H), 3.74 (s, 3H), 3.18 (m, 2H), 1.30 (m, 3H). | 510.20 |
| 15F | 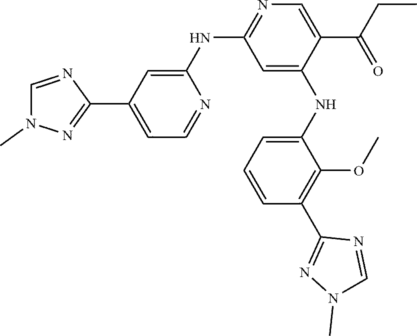 [SMILES: CCC(=O)c1cnc(Nc2cc(-c3ncn(C)n3)ccn2)cc1Nc1cccc(-c2ncn(C)n2)c1OC] | 1H NMR (300 MHz, DMSO-d6) δ 11.33 (s, 1H), 11.11 (s, 1H), 8.98 (m, 1H), 8.68 (m, 1H), 8.60 (m, 1H), 8.44 (m, 1H), 7.84 (m, 1H), 7.73-7.67 (m, 2H), 7.60 (m, 1H), 7.38 (m, 1H), 6.69 (m, 1H), 3.98 (m, 6H), 3.75 (s, 3H), 3.16 (m, 2H), 1.17 (m, 3H). | 511.15 |
| 15G | 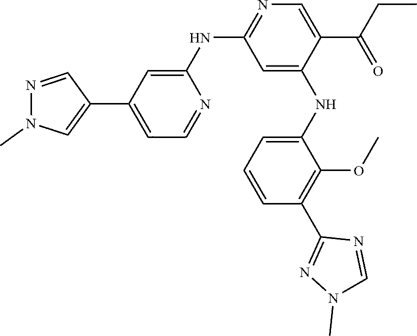 [SMILES: CCC(=O)c1cnc(Nc2cc(-c3cnn(C)c3)ccn2)cc1Nc1cccc(-c2ncn(C)n2)c1OC] | 1H NMR (300 MHz, Methanol-d4) δ 8.85 (m, 1H), 8.50 (m, 1H), 8.17 (m, 2H), 7.95 (m, 1H), 7.72 (m, 1H), 7.65 (m, 1H), 7.35 (m, 2H), 7.25-7.15 (m, 2H), 4.02 (s, 3H), 3.95 (s, 3H), 3.72 (s, 3H), 3.11 (m, 2H), 1.25 (m, 3H). | 510.25 |
| 15H | 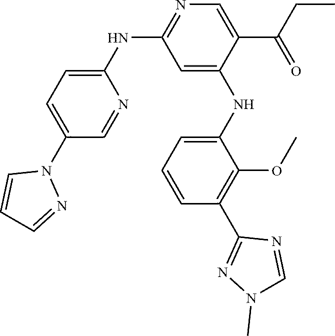 [SMILES: CCC(=O)c1cnc(Nc2ccc(-n3cccn3)cn2)cc1Nc1cccc(-c2ncn(C)n2)c1OC] | 1H NMR (400 MHz, DMSO-d6) δ 11.12 (s, 1H), 10.18 (s, 1H), 8.89 (m, 1H), 8.66 (m, 1H), 8.57 (m, 1H), 8.47 (m, 1H), 8.14 (m, 1H), 7.85-7.63 (m, 5H), 7.35 (m, 1H), 6.55 (m, 1H), 3.96 (s, 3H), 3.75 (s, 3H), 3.11 (m, 2H), 1.14 (m, 3H). | 496.20 |
| 15I | 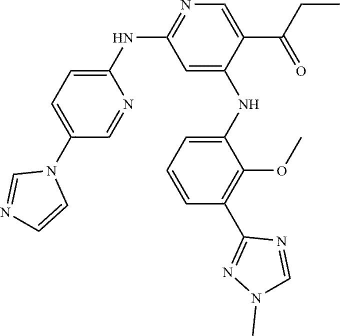 [SMILES: CCC(=O)c1cnc(Nc2ccc(-n3ccnc3)cn2)cc1Nc1cccc(-c2ncn(C)n2)c1OC] | 1H NMR (300 MHz, Methanol-d4) δ 9.51 (m, 1H), 8.97 (m, 1H), 8.77 (m, 1H), 8.65 (m, 1H), 8.23 (m, 1H), 8.10 (m, 1H), 7.92 (m, 1H), 7.82 (m, 1H), 7.64 (m, 1H), 7.43 (m, 1H), 7.26 (m, 1H), 6.64 (m, 1H), 4.06 (s, 3H), 3.79 (s, 3H), 3.17 (m, 2H), 1.32 (m, 3H). | 496.20 |
| 15J | 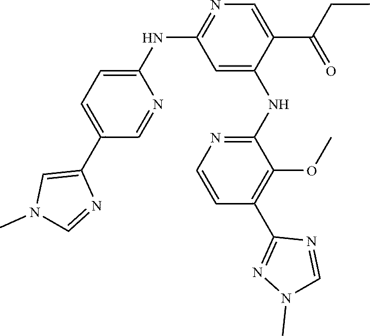 [SMILES: CCC(=O)c1cnc(Nc2ccc(-c3cn(C)cn3)cn2)cc1Nc1nccc(-c2ncn(C)n2)c1OC] | 1H NMR (300 MHz, Methanol-d4) δ 9.32 (m, 1H), 8.89 (m, 1H), 8.68 (m, 1H), 8.54 (m, 1H), 8.23 (m, 1H), 8.03 (m, 1H), 7.77 (m, 1H), 7.70 (m, 1H), 7.48 (m, 2H), 4.07 (s, 3H), 3.94 (s, 3H), 3.80 (s, 3H), 3.10 (m, 2H), 1.27 (m, 3H). | 511.20 |
| 15K | 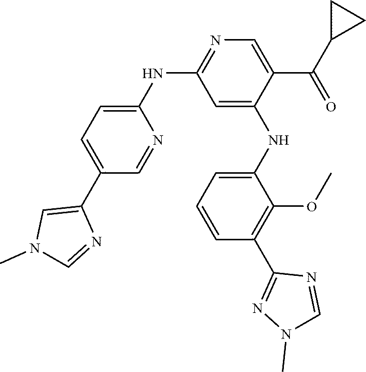 [SMILES: COc1c(Nc2cc(Nc3ccc(-c4cn(C)cn4)cn3)ncc2C(=O)C2CC2)cccc1-c1ncn(C)n1] | 1H NMR (300 MHz, DMSO-d6) δ 11.08 (s, 1H), 9.99 (s, 1H), 9.09 (m, 1H), 8.55 (m, 2H), 7.99 (m, 2H), 7.63-7.59 (m, 5H), 7.32 (m, 1H), 3.95 (s, 3H), 3.72 (s, 3H), 3.68 (s, 3H), 2.95 (m, 1H), 1.07 (m, 4H). | 522.15 |
| 15L | 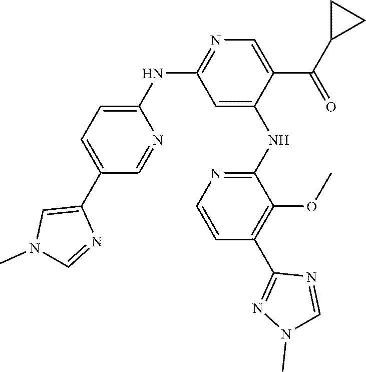 [SMILES: COc1c(-c2ncn(C)n2)ccnc1Nc1cc(Nc2ccc(-c3cn(C)cn3)cn2)ncc1C(=O)C1CC1] | 1H NMR (300 MHz, Methanol-d4) δ 9.30 (m, 1H), 9.09 (m, 1H), 8.68 (m, 1H), 8.55 (m, 1H), 8.22 (m, 1H), 8.04 (m, 1H), 7.75 (m, 1H), 7.69 (m, 1H), 7.47 (m, 2H), 4.06 (s, 3H), 3.90 (s, 3H), 3.80 (s, 3H), 2.87 (m, 1H), 1.22 (m, 2H), 1.10 (m, 2H). | 523.35 |
| 15M | 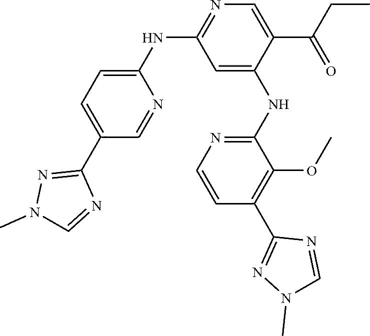 [SMILES: CCC(=O)c1cnc(Nc2ccc(-c3ncn(C)n3)cn2)cc1Nc1nccc(-c2ncn(C)n2)c1OC] | 1H NMR (300 MHz, DMSO-d6) δ 12.37 (s, 1H), 10.33 (s, 1H), 9.51 (m, 1H), 8.96 (m, 1H), 8.90 (m, 1H), 8.67 (m, 1H), 8.54 (m, 1H), 8.22 (m, 2H), 7.86 (m, 1H), 7.51 (m, 1H), 4.00 (s, 3H), 3.93 (m, 6H), 3.12 (m, 2H), 1.16 (m, 3H). | 512.15 |
| 15N | 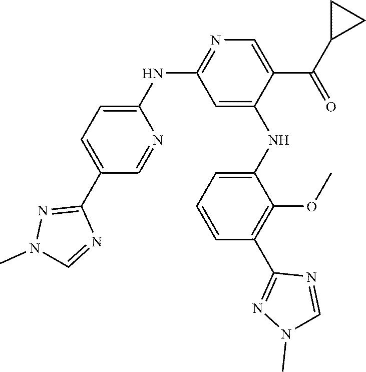 [SMILES: COc1c(Nc2cc(Nc3ccc(-c4ncn(C)n4)cn3)ncc2C(=O)C2CC2)cccc1-c1ncn(C)n1] | 1H NMR (300 MHz, DMSO-d6) δ 11.23 (s, 1H), 11.05 (s, 1H), 9.19 (m, 1H), 8.89 (m, 1H), 8.60 (m, 2H), 8.35 (m, 1H), 7.81 (m, 1H), 7.60 (m, 1H), 7.41-7.30 (m, 2H), 6.90 (m, 1H), 4.00-3.90 (m, 6H), 3.73 (s, 3H), 2.89 (m, 1H), 1.16 (m, 4H). | 523.20 |
| 150 | 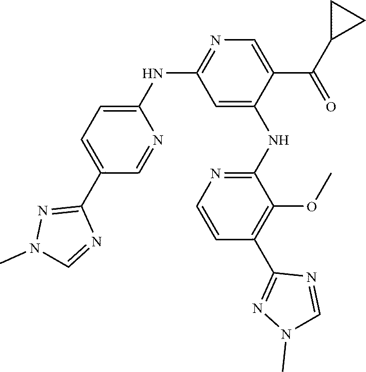 [SMILES: COc1c(-c2ncn(C)n2)ccnc1Nc1cc(Nc2ccc(-c3ncn(C)n3)cn2)ncc1C(=O)C1CC1] | 1H NMR (300 MHz, DMSO-d6) δ 12.33 (s, 1H), 10.36 (s, 1H), 9.52 (m, 1H), 9.20 (m, 1H), 8.90 (m, 1H), 8.66 (m, 1H), 8.54 (m, 1H), 8.22 (m, 2H), 7.85 (m, 1H), 7.50 (m, 1H), 4.00 (s, 3H), 3.94 (s, 3H), 3.88 (s, 3H), 3.01 (m, 1H), 1.15-1.00 (m, 4H). | 524.15 |
| 15P | 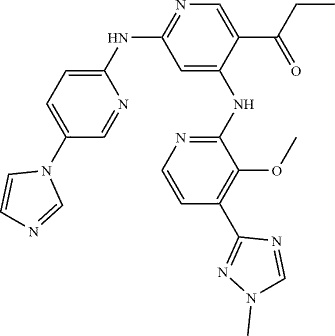 [SMILES: CCC(=O)c1cnc(Nc2ccc(-n3ccnc3)cn2)cc1Nc1nccc(-c2ncn(C)n2)c1OC] | 1H NMR (300 MHz, DMSO-d6) δ 12.39 (s, 1H), 10.33 (s, 1H), 9.53 (m, 1H), 8.96 (m, 1H), 8.66 (m, 2H), 8.27 (m, 2H), 8.04 (m, 1H), 7.88 (m, 1H), 7.78 (m, 1H), 7.49 (m, 1H), 7.16 (m, 1H), 4.00 (s, 3H), 3.93 (s, 3H), 3.13 (m, 2H), 1.16 (m, 3H). | 497.30 |
| 15Q | 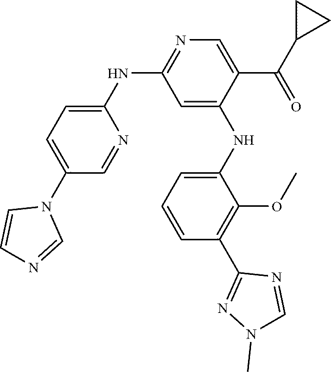 [SMILES: COc1c(Nc2cc(Nc3ccc(-n4ccnc4)cn3)ncc2C(=O)C2CC2)cccc1-c1ncn(C)n1] | 1H NMR (300 MHz, Methanol-d4) δ 9.24 (m, 1H), 9.14 (m, 1H), 8.75 (m, 1H), 8.55 (m, 1H), 8.22 (m, 1H), 8.01 (m, 1H), 7.90 (m, 1H), 7.73 (m, 1H), 7.61 (m, 1H), 7.42 (m, 1H), 7.25 (m, 1H), 6.61 (m, 1H), 4.04 (s, 3H), 3.72 (s, 3H), 2.80 (m, 1H), 1.34 (m, 2H), 1.24 (m, 2H). | 508.30 |
| 15R | 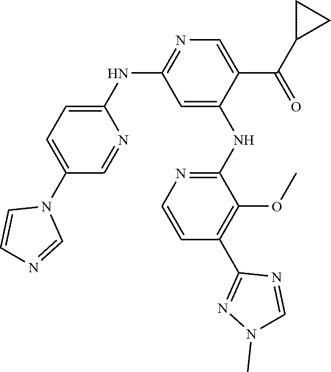 [SMILES: COc1c(-c2ncn(C)n2)ccnc1Nc1cc(Nc2ccc(-n3ccnc3)cn2)ncc1C(=O)C1CC1] | 1H NMR (300 MHz, DMSO-d6) δ 12.33 (s, 1H), 10.35 (s, 1H), 9.53 (m, 1H), 9.20 (m, 1H), 8.84 (m, 2H), 8.27 (m, 2H), 8.04 (m, 1H), 7.88 (m, 1H), 7.77 (m, 1H), 7.47 (m, 1H), 7.15 (m, 1H), 4.11 (s, 3H), 3.99 (s, 3H), 3.00 (m, 1H), 1.15-1.03 (m, 4H). | 509.15 |
| 15S | 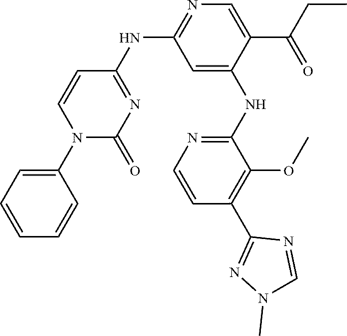 [SMILES: CCC(=O)c1cnc(Nc2ccn(-c3ccccc3)c(=O)n2)cc1Nc1nccc(-c2ncn(C)n2)c1OC] | 1H NMR (300 MHz, DMSO-d6) δ 12.35 (s, 1H), 10.62 (s, 1H), 10.26 (m, 1H), 8.99 (m, 1H), 8.66 (m, 1H), 8.23 (m, 2H), 7.95 (m, 1H), 7.55-7.44 (m, 6H), 6.64 (m, 1H), 3.99 (s, 3H), 3.93 (s, 3H), 3.16 (m, 2H), 1.16 (m, 3H). | 524.30 |
| 15T | 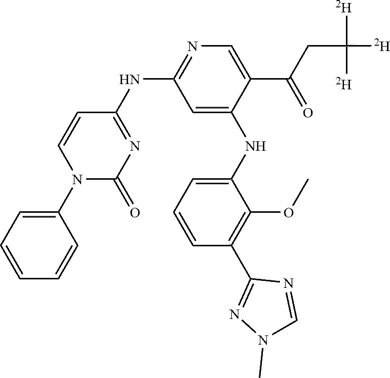 [SMILES: [2H]C([2H])([2H])CC(=O)c1cnc(Nc2ccn(-c3ccccc3)c(=O)n2)cc1Nc1cccc(-c2ncn(C)n2)c1OC] | 1H NMR (400 MHz, Methanol-d4) δ 8.89 (m, 1H), 8.49 (m, 1H), 8.14 (m, 1H), 7.87 (m, 2H), 7.65-7.35 (m, 7H), 6.84 (m, 1H), 4.03 (s, 3H), 3.76 (s, 3H), 3.11 (s, 2H). | 526.25 |
| 15U | 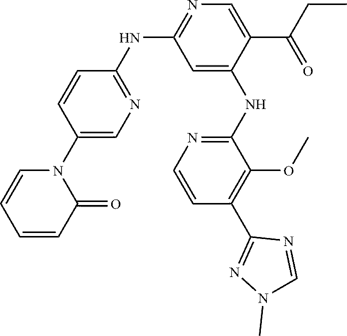 [SMILES: CCC(=O)c1cnc(Nc2ccc(-n3ccccc3=O)cn2)cc1Nc1nccc(-c2ncn(C)n2)c1OC] | 1H NMR (300 MHz, DMSO-d6) δ 12.40 (s, 1H), 10.35 (s, 1H), 9.58 (m, 1H), 8.97 (m, 1H), 8.68 (m, 1H), 8.38 (m, 1H), 8.26 (m, 1H), 7.9-7.85 (m, 2H), 7.62-7.55 (m, 2H), 6.54 (m, 1H), 6.38 (m, 1H), 4.01 (s, 3H), 3.94 (s, 3H), 3.15 (m, 2H), 1.17 (m, 3H). | NA |
| 15V | 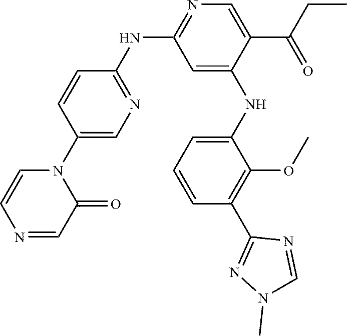 [SMILES: CCC(=O)c1cnc(Nc2ccc(-n3ccncc3=O)cn2)cc1Nc1cccc(-c2ncn(C)n2)c1OC] | 1H NMR (300 MHz, DMSO-d6) δ 11.14 (s, 1H), 11.0 (s, 1H), 8.94 (m, 1H), 8.60 (m, 1H), 8.45 (m, 1H), 8.16 (m, 1H), 7.97 (m, 1H), 7.76 (m, 2H), 7.65 (m, 1H), 7.46-7.35 (m, 3H), 7.1 (m, 1H), 3.96 (s, 3H), 3.75 (s, 3H), 3.13 (m, 2H), 1.16 (m, 3H). | 524.25 |
| 15W | 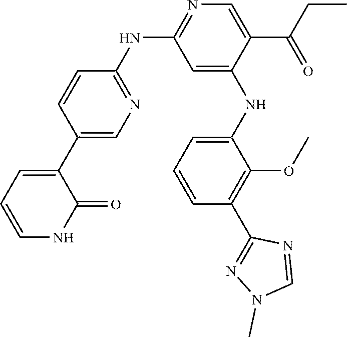 [SMILES: CCC(=O)c1cnc(Nc2ccc(-c3ccc[nH]c3=O)cn2)cc1Nc1cccc(-c2ncn(C)n2)c1OC] | 1H NMR (300 MHz, Methanol-d4) δ 11.32 (s, 1H), 8.93 (m, 1H), 8.80 (m, 1H), 8.53 (m, 1H), 8.22 (m, 1H), 7.85 (m, 2H), 7.68 (m, 1H), 7.50 (m, 1H), 7.38 (m, 1H), 7.05 (m, 1H), 6.56-6.47 (m, 2H), 4.03 (s, 3H), 3.76 (s, 3H), 3.13 (m, 2H), 1.29 (m, 3H). | 523.20 |
| 15X | 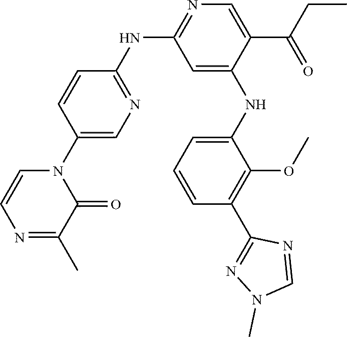 [SMILES: CCC(=O)c1cnc(Nc2ccc(-n3ccnc(C)c3=O)cn2)cc1Nc1cccc(-c2ncn(C)n2)c1OC] | 1H NMR (300 MHz, DMSO-d6) δ 11.13 (s, 1H), 10.26 (s, 1H), 8.90 (m, 1H), 8.58 (m, 1H), 8.32 (m, 1H), 7.95 (m, 1H), 7.87-7.57 (m, 5H), 7.37-7.25 (m, 2H), 3.96 (s, 3H), 3.76 (s, 3H), 3.14 (m, 2H), 2.37 (s, 3H), 1.16 (m, 3H). | 538.35 |
| 15Y | 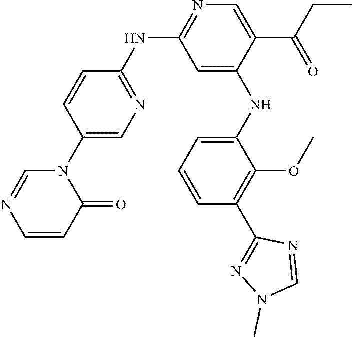 [SMILES: CCC(=O)c1cnc(Nc2ccc(-n3cnccc3=O)cn2)cc1Nc1cccc(-c2ncn(C)n2)c1OC] | 1H NMR (300 MHz, DMSO-d6) δ 12.74 (s, 1H), 11.11 (s, 1H), 8.88 (m, 1H), 8.55 (m, 1H), 8.48 (m, 1H), 8.29 (m, 1H), 7.96 (m, 2H), 7.80 (m, 1H), 7.75-7.58 (m, 3H), 7.32 (m, 1H), 6.51 (m, 1H), 3.93 (s, 3H), 3.73 (s, 3H), 3.13 (m, 2H), 1.14 (m, 3H). | 524.15 |
| 15Z | 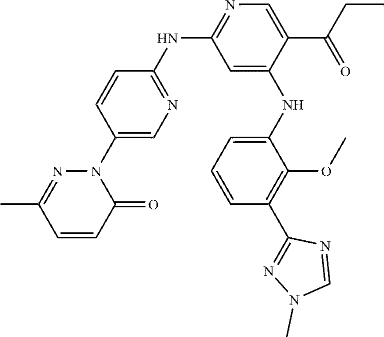 [SMILES: CCC(=O)c1cnc(Nc2ccc(-n3nc(C)ccc3=O)cn2)cc1Nc1cccc(-c2ncn(C)n2)c1OC] | 1H NMR (300 MHz, Methanol-d4) δ 8.93 (m, 1H), 8.74 (m, 1H), 8.55 (m, 1H), 8.17 (m, 1H), 7.89 (m, 1H), 7.62 (m, 1H), 7.50-7.40 (m, 2H), 7.10 (m, 2H), 6.62 (m, 1H), 4.05 (s, 3H), 3.77 (s, 3H), 3.16 (m, 2H), 2.42 (s, 3H), 1.32 (m, 3H). | 538.20 |
| 15AA | 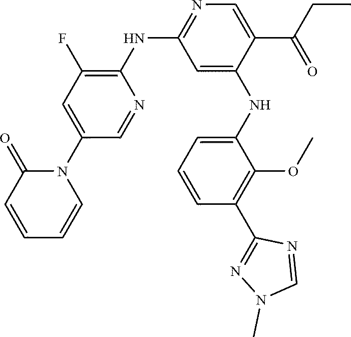 [SMILES: CCC(=O)c1cnc(Nc2ncc(-n3ccccc3=O)cc2F)cc1Nc1cccc(-c2ncn(C)n2)c1OC] | 1H NMR (300 MHz, DMSO-d6) δ 11.12 (s, 1H), 9.67 (s, 1H), 8.87 (m, 1H), 8.56 (m, 1H), 8.16 (m, 1H), 7.96 (m, 2H), 7.74-7.52 (m, 4H), 7.35 (m, 1H), 6.51 (m, 1H), 6.36 (m, 1H), 3.95 (s, 3H), 3.75 (s, 3H), 3.10 (m, 2H), 1.15 (m, 3H). | 541.25 |
| 15BB | 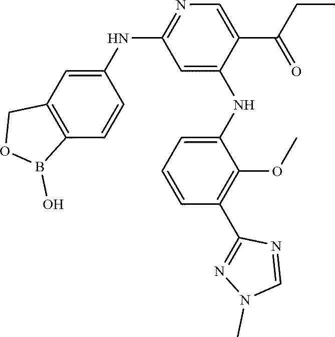 [SMILES: CCC(=O)c1cnc(Nc2ccc3c(c2)COB3O)cc1Nc1cccc(-c2ncn(C)n2)c1OC] | 1H NMR (400 MHz, Methanol-d4) δ 8.73 (m, 1H), 8.49 (m, 1H), 8.09 (m, 1H), 7.67 (m, 1H), 7.56 (m, 3H), 7.27 (m, 2H), 6.46 (m, 1H), 4.98 (s, 2H), 4.02 (s, 3H), 3.70 (s, 3H), 3.16 (m, 2H), 1.23 (m, 3H). | 485.15 |
| 15CC | 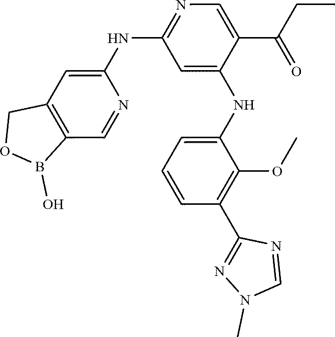 [SMILES: CCC(=O)c1cnc(Nc2cc3c(cn2)B(O)OC3)cc1Nc1cccc(-c2ncn(C)n2)c1OC] | 1H NMR (300 MHz, Methanol-d4) δ 8.96 (m, 1H), 8.62 (m, 1H), 8.54 (m, 1H), 7.86 (m, 1H), 7.59 (m, 1H), 7.40 (m, 1H), 7.06 (m, 1H), 6.52 (m, 1H), 5.12 (s, 2H), 4.05 (s, 3H), 3.73 (s, 3H), 3.16 (m, 2H), 1.27 (m, 3H). | 486.20 |
| 15DD | 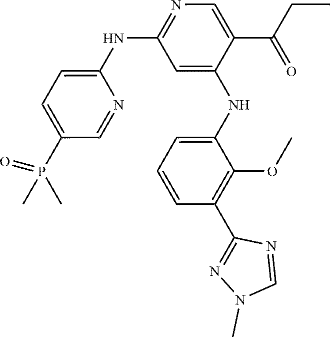 [SMILES: CCC(=O)c1cnc(Nc2ccc(P(C)(C)=O)cn2)cc1Nc1cccc(-c2ncn(C)n2)c1OC] | 1H NMR (300 MHz, Methanol-d4) δ 8.86 (m, 1H), 8.56-8.51 (m, 2H), 7.98 (m, 1H), 7.89 (m, 1H), 7.72 (m, 2H), 7.61 (m, 1H), 7.37 (m, 1H), 4.05 (s, 3H), 3.74 (s, 3H), 3.12 (m, 2H), 1.81 (m, 6H), 1.27 (m, 3H). | 506.30 |

### Example 16

Preparation of 6′-((4-((2-methoxy-3-(1-methyl-1H-1,2,4-triazol-3-yl)phenyl)amino)-5-propionylpyridin-2-yl)amino)-1-methyl-[3,3′-bipyridin]-2(1H)-one (Compound 16A)

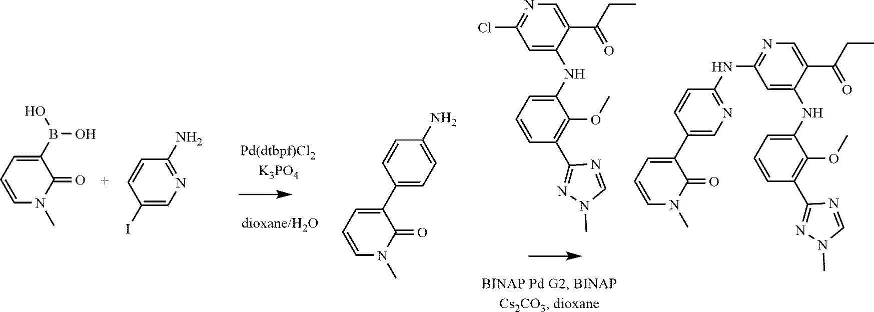

### Step 1: 6′-amino-1-methyl-[3,3′-bipyridin]-2(1H)-one

To a mixture of 4-iodoaniline (200 mg, 0.91 mmol) and (1-methyl-2-oxo-1,2-dihydropyridin-3-yl)boronic acid (168 mg, 1.10 mmol) in 1,4-dioxane (12 mL) was added Pd(dtbpf)Cl2 (60 mg, 0.091 mmol) and tripotassium phosphate (582 mg, 2.74 mmol). The mixture was stirred for 3 hours at 100° C. under N2 atmosphere. The mixture was concentrated under vacuum and the residue was purified by Prep-TLC (dichloromethane:methanol; 10:1) to yield 6′-amino-1-methyl-[3,3′-bipyridin]-2(1H)-one (100 mg, 55% yield) as solid.

### Step 2: 6′-((4-((2-methoxy-3-(1-methyl-1H-1,2,4-triazol-3-yl)phenyl)amino)-5-propionylpyridin-2-yl)amino)-1-methyl-[3,3′-bipyridin1-2(1H)-one

To 1-(6-chloro-4-((2-methoxy-3-(1-methyl-1H-1,2,4-triazol-3-yl)phenyl)amino)pyridin-3-yl)propan-1-one (100 mg, 0.27 mmol) in 1,4-dioxane (10 mL) was added 6′-amino-1-methyl-[3,3′-bipyridin]-2(1H)-one (81 mg, 0.40 mmol); BINAP (17 mg, 0.03 mmol), BINAP Pd G2 (25 mg, 0.03 mmol), and cesium carbonate (175 mg, 0.54 mmol). The mixture was stirred for 12 hours at 90° C. under a nitrogen atmosphere. The mixture was concentrated under vacuum and the residue was purified by prep HPLC to yield 6′-((4-((2-methoxy-3-(1-methyl-1H-1,2,4-triazol-3-yl)phenyl)amino)-5-propionylpyridin-2-yl)amino)-1-methyl-[3,3′-bipyridin]-2(1H)-one (43 mg, 30%) as a solid. ¹H NMR (300 MHz, DMSO-d6) δ 11.40 (s, 1H), 11.12 (s, 1H), 9.00 (s, 1H), 8.85 (d, J = 2.3 Hz, 1H), 8.61 (s, 1H), 8.26 (dd, J = 8.7, 2.4 Hz, 1H), 7.82 (dtd, J = 14.6, 7.5, 7.1, 1.9 Hz, 3H), 7.61 (dd, J = 7.9, 1.7 Hz, 1H), 7.45 - 7.28 (m, 1H), 7.24 - 7.10 (m, 1H), 6.70 (s, 1H), 6.41 (t, J = 6.9 Hz, 1H), 3.98 (s, 3H), 3.77 (s, 3H), 3.55 (s, 3H), 3.16 (q, J = 7.1 Hz, 2H), 1.18 (t, J = 7.1 Hz, 3H).

$$\text{LC-MS: (ES m/z): [M+H]+ 537} \text{.20} \text{.}$$

### Example 17

Preparation of 1-(6-((6-((dimethylamino)methyl)pyrimidin-4-yl)amino)-4-((2-methoxy-3-(1-methyl-1H-1,2,4-triazol-3-yl)phenyl)amino)pyridin-3-yl)propan-1-one (Comopund 17A)

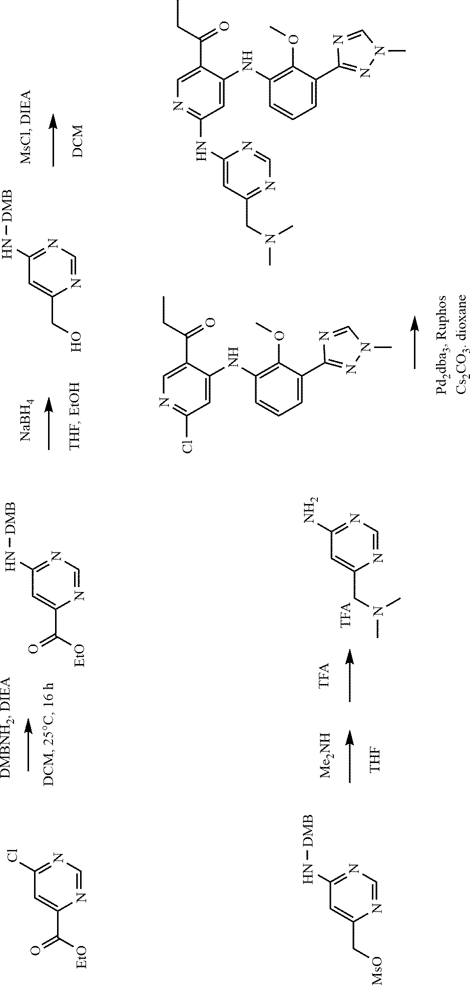

### Step 1: Ethyl 6-((3,4-dimethylbenzyl)amino)pyrimidine-4-carboxylate

A mixture of ethyl 6-chloropyrimidine-4-carboxylate (1 g, 5.36 mmol), 2,4-dimethoxybenzylamine (896 mg, 5.36 mmol) and diisopropylethylamine (1.39 g, 10.6 mmol) in dichloromethane (12 mL) was stirred at 25° C. for 16 hours. Water (50 mL) was added to the reaction mixture and extracted with ethyl acetate (3 × 100 mL). The combined organic layer was washed with brine (60 mL), dried over sodium sulfate and concentrated in vacuo. The residue was purified by column chromatography on silica gel eluting with ethyl acetate in dichloromethane (0 to 29%) to yield ethyl 6-((3,4-dimethylbenzyl)amino)pyrimidine-4-carboxylate (1 g, 58%) as an oil.

### Step 2: 6-((2,4-dimethoxybenzyl)amino)pyrimidine-4-carboxylic Acid

To a solution of ethyl 6-((3,4-dimethylbenzyl)amino)pyrimidine-4-carboxylate (1 g, 3.15 mmol) in tetrahydrofuran (9 mL) and ethanol (3 mL) was added sodium borohydride (252 mg, 6.30 mmol). The reaction mixture was stirred at 25° C. for 2 hours. The mixture was concentrated in vacu, added water (15 mL) and then adjusted to pH 4~5 with hydrochloric acid (1 M, 30 mL). The solid was collected by filtration to yield 6-((2,4-dimethoxybenzyl)amino)pyrimidine-4-carboxylic acid (830 mg, 91%) as a solid.

### Step 3: (6-((2,4-dimethoxybenzyl)amino)pyrimidin-4-yl)methyl methanesulfonate

To a solution of 6-((2,4-dimethoxybenzyl)amino)pyrimidine-4-carboxylic acid (500 mg, 1.82 mmol) in dichloromethane (10 mL) was added diisopropylethylamine (587 mg, 4.54 mmol) and methanesulfonyl chloride (312 mg, 2.72 mmol) at 0° C. The reaction mixture was stirred at 0° C. for 2 hours then quenched with water (5 mL) and extracted with methanol in dichloromethane (10:90; 3 × 50 mL). The combined organic layer was dried over sodium sulfate and concentrated in vacuo to yield (6-((2,4-dimethoxybenzyl)amino)pyrimidin-4-yl)methyl methanesulfonate (600 mg, crude theoretical) as a solid.

### Step 4: N-(2,4-dimethoxybenzyl)-6-((dimethylamino)methyl)pyrimidin-4-amine

To a solution of (6-((2,4-dimethoxybenzyl)amino)pyrimidin-4-yl)methyl methanesulfonate (600 mg, 1.70 mmol) in tetrahydrofuran (5 mL) was added dimethylamine (2 M in THF, 4.24 mL). The reaction mixture was stirred at 25° C. for 2 hours. The mixture was concentrated under a vacuum. To the residue was added water (5 mL) and extracted with methanol in dichloromethane (10:90; 4 × 50 mL). The combined organic layer was dried over sodium sulfate and concentrated in vacuo. The residue was purified by column chromatography on silica gel eluting with methanol in dichloromethane (0 to 15%) to yield N-(2,4-dimethoxybenzyl)-6-((dimethylamino)methyl)pyrimidin-4-amine (350 mg, 68%) as a solid.

### Step 5: 6-((dimethylamino)methyl)pyrimidin-4-amine 2,2,2-trifluoroacetate

A mixture of N-(2,4-dimethoxybenzyl)-6-((dimethylamino)methyl)pyrimidin-4-amine (350 mg, 1.16 mmol) and trifluoroacetic acid (5 mL) was stirred at 100° C. for 2 hours. The reaction mixture was concentrated in vacuo to yield 6-((dimethylamino)methyl)pyrimidin-4-amine 2,2,2-trifluoroacetate (500 mg, crude) as a solid.

### Step 6: 1-(6-((6-((dimethylamino)methyl)pyrimidin-4-yl)amino)-4-((2-methoxy-3-(1-methyl-1H-1,2,4-triazol-3-yl)phenyl)amino)pyridin-3-yl)propan-1-one

A mixture of 1-(6-chloro-4-((2-methoxy-3-(1-methyl-1H-1,2,4-triazol-3-yl)phenyl)amino)pyridin-3-yl)propan-1-one (70 mg, 0.188 mmol), 6-((dimethylamino) methyl)pyrimidin-4-amine 2,2,2-trifluoroacetate (43 mg, 0.161 mmol), tris(dibenzylideneacetone)dipalladium(0) (17 mg, 0.018 mmol), Ruphos (17 mg, 0.037 mmol) and cesium carbonate (307 mg, 0.941 mmol) in 1,4-dioxane (5 mL) was stirred at 100° C. for 2 hours under N2. The reaction mixture was cooled to room temperature, added water (15 mL) and extracted with ethyl acetate (3 × 40 mL). The combined organic layer was washed with brine (30 mL), dried over sodium sulfate and concentrated in vacuo. The residue was purified by Pre-HPLC to yield 1-(6-((6-((dimethylamino)methyl)pyrimidin-4-yl)amino)-4-((2-methoxy-3-(1-methyl-1H-1,2,4-triazol-3-yl)phenyl)amino)pyridin-3-yl)propan-1-one (44 mg, 48%) as a solid. ¹HNMR (300 MHz, DMSO-d6) δ 11.10 (s, 1H), 10.32 (s, 1H), 8.91 (s, 1H), 8.67-8.45 (m, 2H), 7.85 (s, 1H), 7.76 (s, 1H), 7.72-7.57 (m, 2H), 7.39-7.23 (m, 1H), 3.96 (s, 3H), 3.75 (s, 3H), 3.42 (s, 2H), 3.13 (q, J = 6.9 Hz, 2H), 2.22 (s, 6H), 1.13 (t, J = 7.2 Hz, 3H).

$$\text{ESI-MS [M+H]+: 488} \text{.20} \text{.}$$

### Example 18

Preparation of 1-(6-((2-(3-hydroxypropoxy)pyrimidin-4-yl)amino)-4-((2-methoxy-3-(1-methyl-1H-1,2,4-triazol-3-yl)phenyl)amino)pyridin-3-yl)propan-1-one (Compound 18A)

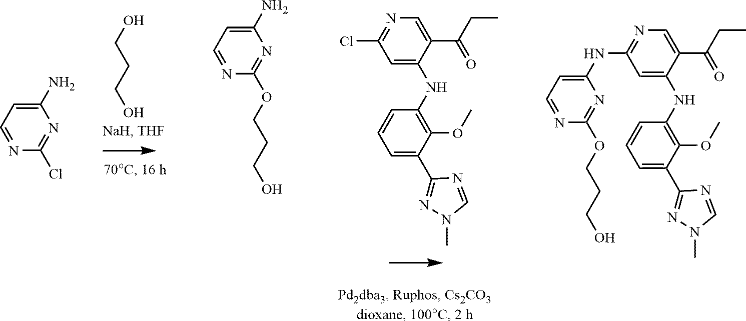

### Step 1: 3-((4-aminopyrimidin-2-yl)oxy)propan-1-ol

To a mixture of 2-chloropyrimidin-4-amine (500 mg, 3.86 mmol) and propane-1,3-diol (881 mg, 11.58 mmol) in tetrahydrofuran (10 mL) was added sodium hydride (464 mg, 11.58 mmol, 60% purity). The reaction mixture was stirred at 70° C. for 16 hours. The cooled mixture was diluted with water (30 mL) and extracted with ethyl acetate (3 × 40 mL). The combined organic layer was washed with brine (30 mL), dried over sodium sulfate and concentrated in vacuo. The residue was purified by column chromatography on silica gel eluting with ethyl acetate in petroleum ether (0 to 80%) to yield 3-((4-aminopyrimidin-2-yl)oxy)propan-1-ol (150 mg, 16%) as a solid.

### Step 2: 1-(6-((2-(3-hydroxypropoxy)pyrimidin-4-yl)amino)-4-((2-methoxy-3-(1-methyl-1H-1,2,4-triazol-3-yl)phenyl)amino)pyridin-3-yl)propan-1-one

A mixture of 1-(6-chloro-4-((2-methoxy-3-(1-methyl-1H-1,2,4-triazol-3-yl)phenyl)amino)pyridin-3-yl)propan-1-one (100 mg, 0.27 mmol), 3-((4-aminopyrimidin-2-yl)oxy)propan-1-ol (68 mg, 0.40 mmol), tris(dibenzylideneacetone)dipalladium(0) (28 mg, 0.027 mmol), Ruphos (25 mg, 0.54 mmol) and cesium carbonate (263 mg, 0.81 mmol) in 1,4-dioxane (4 mL) was stirred at 100° C. for 2 hours under N2. The cooled reaction mixture was quenched with water (10 mL) and extracted with ethyl acetate (3 × 30 mL), the combined organic layer was washed with brine (20 mL), dried over sodium sulfate and concentrated in vacuo. The residue was purified by prep-HPLC to yield 1-(6-((2-(3-hydroxypropoxy)pyrimidin-4-yl)amino)-4-((2-methoxy-3-(1-methyl-1H-1,2,4-triazol-3-yl)phenyl)amino)pyridin-3-yl)propan-1-one (51 mg, 37%) as a solid. ¹H NMR (400 MHz, DMSO-d6) δ 11.12 (s, 1H), 10.35 (s, 1H), 8.89 (s, 1H), 8.57 (s, 1H), 8.21 (d, J = 5.8 Hz, 1H), 8.00 (s, 1H), 7.73-7.59 (m, 2H), 7.27 (t, J = 8.0 Hz, 1H), 7.10 (d, J = 5.8 Hz, 1H), 4.50 (t, J = 5.2 Hz, 1H), 4.12 (t, J = 6.4 Hz, 2H), 3.96 (s, 3H), 3.74 (s, 3H), 3.48 (q, J = 6.2 Hz, 2H), 3.12 (q, J = 7.2 Hz, 2H), 1.80-1.71(m, 2H), 1.13 (t, J = 7.2 Hz, 3H).

$$\text{ESI-MS [M+H]+: 505} \text{.25} \text{.}$$

### Preparation of Compounds 18B-18DDDD

Compounds 18B-18DDDD, as indicated in TABLE 13, were prepared in a similar manner and according to the general synthetic schemes and procedures described herein.

TABLE 13

COMPOUNDS 18B THROUGH 18DDDD

| Cmpd. No. | Structure | ¹H NMR | MS (M+H)⁺ |
|---|---|---|---|
| 18B | 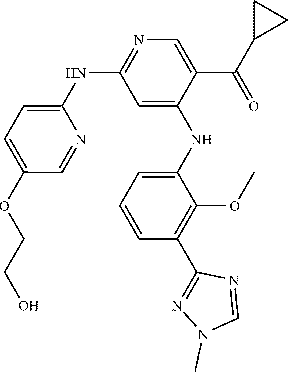 [SMILES: COc1c(Nc2cc(Nc3ccc(OCCO)cn3)ncc2C(=O)C2CC2)cccc1-c1ncn(C)n1] | 1H NMR (400 MHz, DMSO-d6) δ 11.06 (s, 1H), 9.8 (s, 1H), 9.07 (m, 1H), 8.56 (m, 1H), 7.92 (m, 1H), 7.66 (m, 1H), 7.68-7.59 (m, 3H), 7.40 (m, 1H), 7.32 (m, 1H), 4.88 (m, 1H), 4.01 (m, 2H), 3.95 (s, 3H), 3.71 (m, 5H), 2.94 (m, 1H), 1.08-1.00 (m, 4H). | 502.3 |
| 18C | 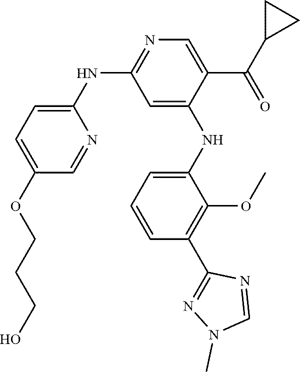 [SMILES: COc1c(Nc2cc(Nc3ccc(OCCCO)cn3)ncc2C(=O)C2CC2)cccc1-c1ncn(C)n1] | 1H NMR (300 MHz, DMSO-d6) δ 11.07 (s, 1H), 9.90 (s, 1H), 9.08 (m, 1H), 8.57 (m, 1H), 7.92 (m, 1H), 7.65 (m, 2H), 7.52-7.30 (m, 4H), 4.58 (m, 1H), 4.07 (m, 2H), 3.97 (s, 3H), 3.72 (s, 3H), 3.58 (m, 2H), 2.94 (m, 1H), 1.85 (m, 2H), 1.14-0.97 (m, 4H). | 516.25 |
| 18D | 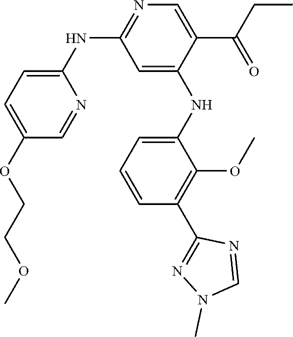 [SMILES: CCC(=O)c1cnc(Nc2ccc(OCCOC)cn2)cc1Nc1cccc(-c2ncn(C)n2)c1OC] | 1H NMR (400 MHz, DMSO-6) δ 11.11 (s, 1H), 9.81 (s, 1H), 8.83 (s, 1H), 8.57 (s, 1H), 7.92 (d, J = 3.0 Hz, 1H), 7.80 (s, 1H), 7.71 - 7.64 (m, 1H), 7.64 - 7.55 (m, 2H), 7.40 (dd, J = 9.1, 3.1 Hz, 1H), 7.33 (t, J = 7.9 Hz, 1H), 4.16 - 4.09 (m, 2H), 3.96 (s, 3H), 3.76 (s, 3H), 3.65 (dd, J = 5.6, 3.4 Hz, 2H), 3.32 (m, 3H), 3.08 (q, J = 7.2 Hz, 2H), 1.13 (t, J = 7.2 Hz, 3H). | 504.2 |
| 18E | 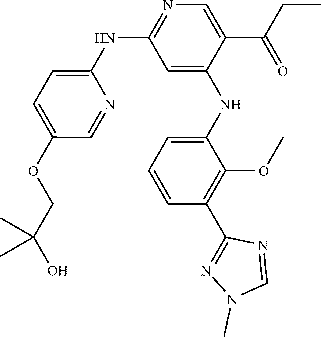 [SMILES: CCC(=O)c1cnc(Nc2ccc(OCC(C)(C)O)cn2)cc1Nc1cccc(-c2ncn(C)n2)c1OC] | 1H NMR (400 MHz, Methanol-d4) δ 8.77 (s, 1H), 8.50 (s, 1H), 7.95 (t, J = 1.8 Hz, 1H), 7.71 (dd, J = 8.0, 1.5 Hz, 1H), 7.66 (dd, J = 7.8, 1.5 Hz, 1H), 7.61 (s, 1H), 7.39 (s, 1H), 7.36 (s, 1H) 7.33 (t, J = 7.9 Hz, 1H), 4.04 (s, 3H), 3.83 (s, 2H), 3.74 (s, 3H), 3.07 (q, J = 7.3 Hz, 2H), 1.34 (s, 6H), 1.26 (t, J = 7.3 Hz, 3H). | 518.3 |
| 18F | 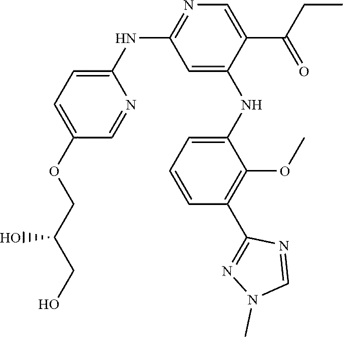 [SMILES: CCC(=O)c1cnc(Nc2ccc(OC[C@@H](O)CO)cn2)cc1Nc1cccc(-c2ncn(C)n2)c1OC] | 1H NMR (400 MHz, DMSO-d6) δ 11.21 (s, 1H), 11.06 (s, 1H), 8.94 (m, 1H), 8.57 (m, 1H), 8.01 (m, 1H), 7.81 (m, 1H), 7.60-7.54 (m, 2H), 7.36 (m, 1H), 7.05 (m, 1H), 6.56 (m, 1H), 4.07 (m, 1H), 3.97 (m, 4H), 3.79 (m, 1H), 3.72 (s, 3H), 3.43 (m, 2H), 3.09 (m, 2H), 1.16 (m, 3H). | 520.3 |
| 18G | 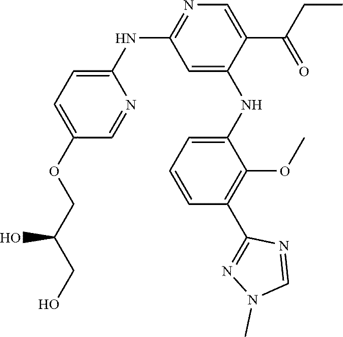 [SMILES: CCC(=O)c1cnc(Nc2ccc(OC[C@H](O)CO)cn2)cc1Nc1cccc(-c2ncn(C)n2)c1OC] | 1H NMR (300 MHz, Methanol-d4) δ 8.64 (s, 1H), 8.38 (m, 1H), 7.83 (m, 1H), 7.6-7.45 (m, 3H), 7.26-7.17 (m, 3H), 4.01-3.82 (m, 6H), 3.62-3.51 (m, 5H), 2.98 (m, 2H), 1.14 (m, 3H). | 520.25 |
| 18H | 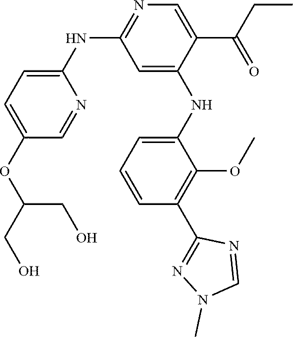 [SMILES: CCC(=O)c1cnc(Nc2ccc(OC(CO)CO)cn2)cc1Nc1cccc(-c2ncn(C)n2)c1OC] | 1H NMR (300 MHz, Methanol-d4) δ 8.77 (s, 1H), 8.50 (m, 1H), 8.02 (m, 1H), 7.72-7.61 (m, 3H), 7.45 (m, 1H), 7.39-7.29 (m, 2H), 4.28 (m, 1H), 4.04 (s, 3H), 3.80-3.73 (m, 7H), 3.07 (m, 2H), 1.26 (m, 3H). | 520.25 |
| 18I | 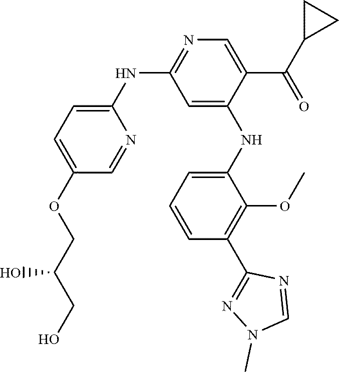 [SMILES: COc1c(Nc2cc(Nc3ccc(OC[C@@H](O)CO)cn3)ncc2C(=O)C2CC2)cccc1-c1ncn(C)n1] | 1H NMR (300 MHz, DMSO-d6) δ 11.17 (s, 1H), 11.0 (s, 1H), 9.18 (s, 1H), 8.59 (m, 1H), 8.04 (m, 1H), 7.83 (m, 1H), 7.61 (m, 2H), 7.38 (m, 1H), 7.12 (m, 1H), 6.62 (m, 1H), 4.11 (m, 1H), 4.0-3.95 (m, 4H), 3.82 (m, 1H), 3.73 (s, 3H), 3.46 (m, 2H), 2.83 (m, 1H), 1.16 (m, 4H). | 532.25 |
| 18J | 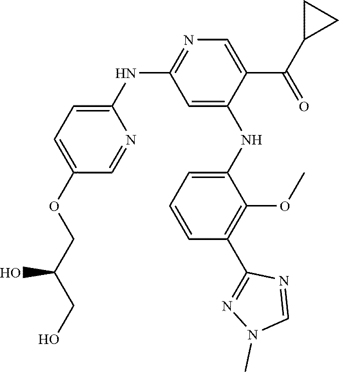 [SMILES: COc1c(Nc2cc(Nc3ccc(OC[C@H](O)CO)cn3)ncc2C(=O)C2CC2)cccc1-c1ncn(C)n1] | 1H NMR (300 MHz, Methanol-d4) δ 8.95 (m, 1H), 8.50 (m, 1H), 7.93 (m, 1H), 7.7-7.56 (m, 3H), 7.38-7.25 (m, 3H), 4.11 (m, 1H), 4.01-3.92 (m, 5H), 3.70-3.62 (m, 5H), 2.89 (m, 1H), 1.19 (m, 2H), 1.04 (m, 2H). | 532.25 |
| 18K | 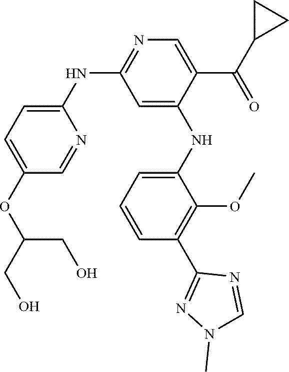 [SMILES: COc1c(Nc2cc(Nc3ccc(OC(CO)CO)cn3)ncc2C(=O)C2CC2)cccc1-c1ncn(C)n1] | 1H NMR (400 MHz, Methanol-d4) δ 8.96 (m, 1H), 8.50 (m, 1H), 8.03 (m, 1H), 7.72-7.58 (m, 3H), 7.46 (m, 1H), 7.40 (m, 1H), 7.31 (m, 1H), 4.31 (m, 1H), 4.04 (s, 3H), 3.82-3.7 (m, 7H), 2.81 (m, 1H), 1.22 (m, 2H), 1.07 (m, 2H). | 532.20 |
| 18L | 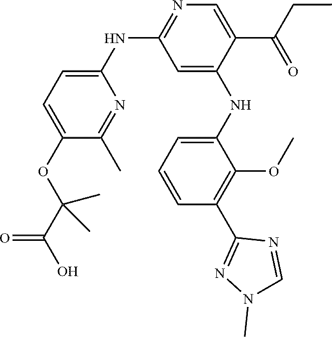 [SMILES: CCC(=O)c1cnc(Nc2ccc(OC(C)(C)C(=O)O)c(C)n2)cc1Nc1cccc(-c2ncn(C)n2)c1OC] | 1H NMR (400 MHz, Methanol-d4) δ 8.88 (m, 1H), 8.51 (m, 1H), 7.75 (m, 1H), 7.63 (m, 1H), 7.32 (m, 2H), 7.05 (m, 1H), 6.78 (m, 1H), 4.04 (s, 3H), 3.73 (s, 3H), 3.08 (m, 2H), 2.43 (s, 3H), 1.56 (s, 6H), 1.24 (m, 3H). | 546.30 |
| 18M | 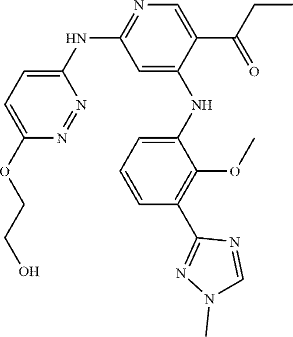 [SMILES: CCC(=O)c1cnc(Nc2ccc(OCCO)nn2)cc1Nc1cccc(-c2ncn(C)n2)c1OC] | 1H NMR (400 MHz, Methanol-d4) δ 8.82 (m, 1H), 8.50 (m, 1H), 8.04 (m, 1H), 7.68 (m, 2H), 7.37-7.30 (m, 2H), 7.15 (m, 1H), 4.46 (m, 2H), 4.04 (s, 3H), 3.92 (m, 2H), 3.74 (s, 3H), 3.15 (m, 2H), 1.26 (m, 3H). | 491.20 |
| Cmpd. No. | Structure | ¹H NMR | MS (M+H)⁺ |
| 18N | 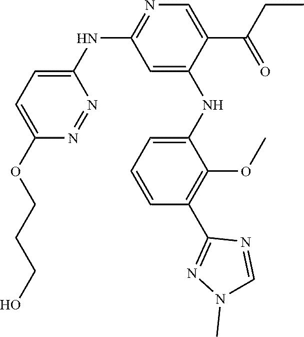 [SMILES: CCC(=O)c1cnc(Nc2ccc(OCCCO)nn2)cc1Nc1cccc(-c2ncn(C)n2)c1OC] | 1H NMR (300 MHz, DMSO-d6) δ 11.11 (s, 1H), 10.12 (s, 1H), 8.85 (m, 1H), 8.58 (m, 1H), 8.01 (m, 1H), 7.65 (m, 3H), 7.27 (m, 1H), 7.16 (m, 1H), 4.58 (m, 1H), 4.39 (m, 2H), 3.97 (s, 3H), 3.78 (s, 3H), 3.56 (m, 2H), 3.10 (m, 2H), 1.89 (m, 2H), 1.13 (m, 3H). | 505.20 |
| 180 | 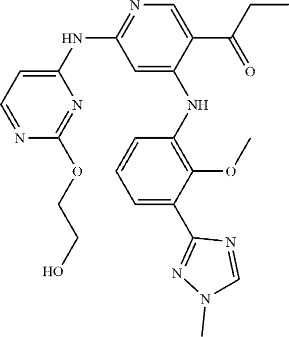 [SMILES: CCC(=O)c1cnc(Nc2ccnc(OCCO)n2)cc1Nc1cccc(-c2ncn(C)n2)c1OC] | 1H NMR (300 MHz, DMSO-d6) δ 11.10 (s, 1H), 10.35 (s, 1H), 8.90 (m, 1H), 8.57 (m, 1H), 8.21 (m, 1H), 8.01 (m, 1H), 7.66 (m, 2H), 7.36 (m, 1H), 7.10 (m, 1H), 4.78 (m, 1H), 4.06 (m, 2H), 3.96 (s, 3H), 3.74 (s, 3H), 3.59 (m, 2H), 3.12 (m, 2H), 1.14 (m, 3H). | 491.25 |
| 18P | 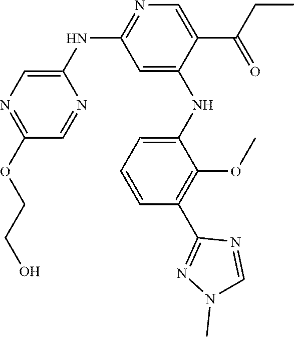 [SMILES: CCC(=O)c1cnc(Nc2cnc(OCCO)cn2)cc1Nc1cccc(-c2ncn(C)n2)c1OC] | 1H NMR (400 MHz, Methanol-d4) δ 8.80 (m, 1H), 8.51 (m, 2H), 7.92 (m, 1H), 7.65 (m, 2H), 7.31 (m, 2H), 4.37 (m, 2H), 4.04 (s, 3H), 3.88 (m, 2H), 3.73 (s, 3H), 3.11 (m, 2H), 1.3 (m, 3H). | 491.30 |
| 18Q | 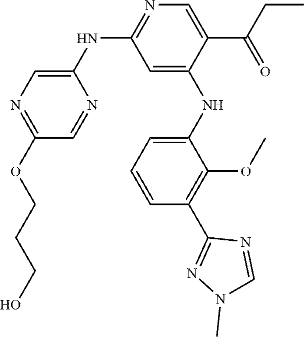 [SMILES: CCC(=O)c1cnc(Nc2cnc(OCCCO)cn2)cc1Nc1cccc(-c2ncn(C)n2)c1OC] | 1H NMR (300 MHz, Methanol-d4) δ 8.82 (m, 1H), 8.53 (m, 1H), 8.25 (m, 1H), 7.98 (m, 1H), 7.78 (m, 1H), 7.64 (m, 1H), 7.36 (m, 1H), 6.90 (m, 1H), 4.44 (m, 2H), 4.05 (s, 3H), 3.78-3.70 (m, 5H), 3.12 (m, 2H), 1.27 (m, 3H). | 505.20 |
| 18R | 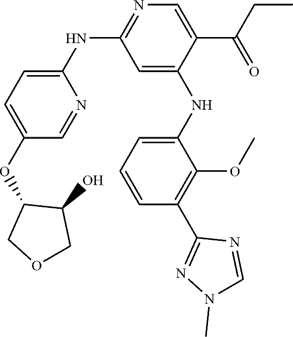 [SMILES: CCC(=O)c1cnc(Nc2ccc(O[C@H]3COC[C@@H]3O)cn2)cc1Nc1cccc(-c2ncn(C)n2)c1OC] | 1H NMR (300 MHz, Methanol-d4) δ 8.77 (s, 1H), 8.50 (s, 1H), 7.98 - 7.91 (m, 1H), 7.71 (dd, J = 8.0, 1.6 Hz, 1H), 7.70 - 7.59 (m, 2H), 7.43 - 7.37 (m, 2H), 7.33 (t, J = 7.9 Hz, 1H), 4.71 (d, J = 4.3 Hz, 1H), 4.34 (d, J = 4.1 Hz, 1H), 4.18 (dd, J = 10.2, 4.3 Hz, 1H), 4.04 (dd, J = 9.7, 4.2 Hz, 1H), 4.04 (s, 3H), 3.91 (d, J = 10.2 Hz, 1H), 3.77 (dd, J = 9.7, 1.8 Hz, 1H), 3.74 (s, 3H), 3.07 (q, J = 7.3 Hz, 2H), 1.25 (t, J = 7.3 Hz, 3H). | 532.2 |
| 18S | 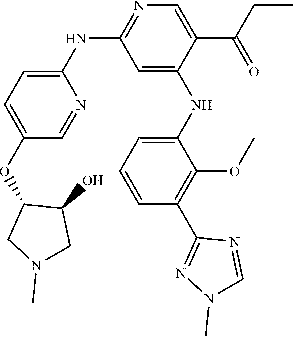 [SMILES: CCC(=O)c1cnc(Nc2ccc(O[C@H]3CN(C)C[C@@H]3O)cn2)cc1Nc1cccc(-c2ncn(C)n2)c1OC] | 1H NMR (400 MHz, Methanol-d4) δ 8.77 (s, 1H), 8.50 (s, 1H), 7.98 (s, 1H), 7.75-7.70 (m, 1H), 7.68 - 7.62 (m, 2H), 7.40 (d, J = 2.8 Hz, 2H), 7.33 (t, J = 7.9 Hz, 1H), 4.60 (d, J = 5.8 Hz, 1H), 4.30 (t, J = 5.6 Hz, 1H), 4.04 (s, 3H), 3.74 (s, 3H), 3.17 - 3.02 (m, 4H), 2.85 (m, 1H), 2.49-2.44 (m, 1H), 2.40 (s, 3H), 1.25 (t, J = 7.3, 3H). | 545.3 |
| 18T | 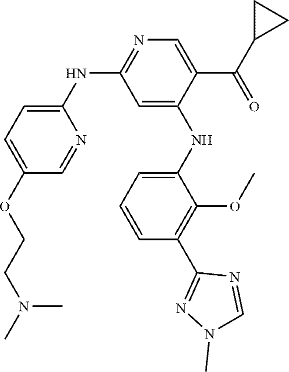 [SMILES: COc1c(Nc2cc(Nc3ccc(OCCN(C)C)cn3)ncc2C(=O)C2CC2)cccc1-c1ncn(C)n1] | 1H NMR (300 MHz, DMSO-d6) δ 11.09 (s, 1H), 9.84 (s, 1H), 9.08 (m, 1H), 8.57 (m, 1H), 7.93 (m, 1H), 7.81 (m, 1H), 7.68 (m, 1H), 7.58 (m, 2H), 7.43-7.31 (m, 2H), 4.10 (m, 2H), 3.97 (s, 3H), 3.73 (s, 3H), 2.95 (m, 1H), 2.63 (m, 2H), 2.24 (s, 6H), 1.11-1.00 (m, 4H). | 529.3 |
| 18U | 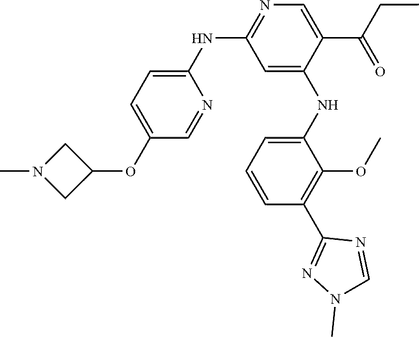 [SMILES: CCC(=O)c1cnc(Nc2ccc(OC3CN(C)C3)cn2)cc1Nc1cccc(-c2ncn(C)n2)c1OC] | 1H NMR (300 MHz, DMSO-d6) δ 11.08 (s, 1H), 9.80 (s, 1H), 8.82 (m, 1H), 8.57 (m, 1H), 7.80-7.56 (m, 5H), 7.33 (m, 2H), 4.77 (m, 1H), 3.96 (s, 3H), 3.77 (m, 5H), 3.05 (m, 4H), 2.32 (s, 3H), 1.15 (m, 3H). | 515.20 |
| 18V | 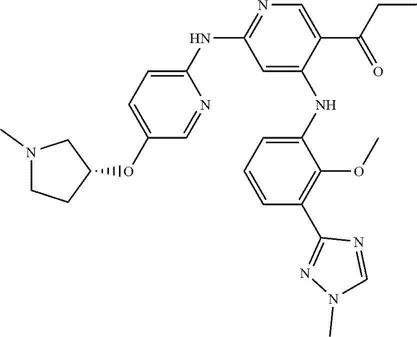 [SMILES: CCC(=O)c1cnc(Nc2ccc(O[C@@H]3CCN(C)C3)cn2)cc1Nc1cccc(-c2ncn(C)n2)c1OC] | 1H NMR (300 MHz, D₂O) δ 8.96 (m, 1H), 8.56 (m, 1H), 7.86 (m, 1H), 7.49 (m, 1H), 7.38 (m, 2H), 7.16 (m, 1H), 6.87 (m, 1H), 6.34 (m, 1H), 5.10 (m, 1H), 3.98 (s, 3H), 3.79 (m, 2H), 3.47 (s, 3H), 3.35-3.15 (m, 2H), 2.97 (m, 3H), 2.86 (m, 2H), 2.58 (m, 1H), 2.3-2.1 (m, 1H), 0.93 (m, 3H). | 529.25 |
| 18W | 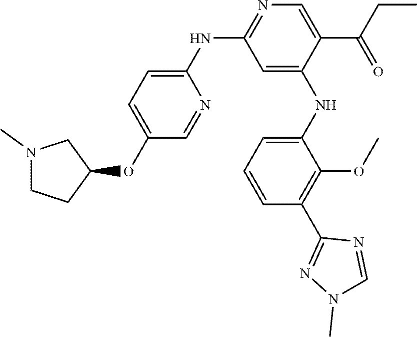 [SMILES: CCC(=O)c1cnc(Nc2ccc(O[C@H]3CCN(C)C3)cn2)cc1Nc1cccc(-c2ncn(C)n2)c1OC] | 1H NMR (300 MHz, DMSO-d6) δ 11.21 (s, 1H), 11.11 (s, 1H), 10.50 (s, 1H), 8.96 (m, 1H), 8.60 (m, 1H), 8.01 (m, 1H), 7.83 (m, 1H), 7.62 (m, 2H), 7.38 (m, 1H), 7.18 (m, 1H), 6.76 (m, 1H), 5.23 (m, 1H), 4.77 (m, 1H), 3.96 (s, 3H), 3.8-3.7 (m, 5H), 3.32 (m, 1H), 3.15 (m, 2H), 2.96-2.87 (m, 3H), 2.1 (m, 1H), 1.17 (m, 3H). | 529.25 |
| 18X | 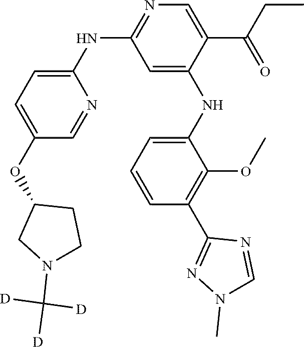 [SMILES: [2H]C([2H])([2H])N1CC[C@@H](Oc2ccc(Nc3cc(Nc4cccc(-c5ncn(C)n5)c4OC)c(C(=O)CC)cn3)nc2)C1] | 1H NMR (300 MHz, Methanol-d4) δ 8.77 (m, 1H), 8.50 (m, 1H), 7.87 (m, 1H), 7.75-7.58 (m, 3H), 7.40-7.28 (m, 3H), 5.9 (m, 1H), 4.04 (s, 3H), 3.74 (s, 3H), 3.09 (m, 2H), 2.98-2.82 (m, 3H), 2.52 (m, 1H), 2.38 (m, 1H), 2.01 (m, 1H), 1.26 (m, 3H). | 532.30 |
| 18Y | 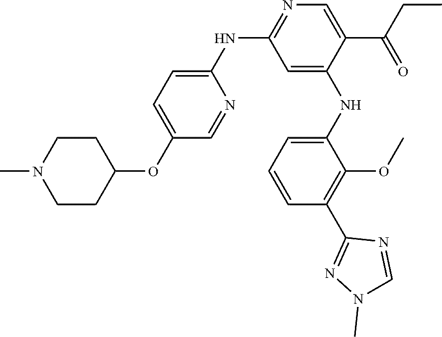 [SMILES: CCC(=O)c1cnc(Nc2ccc(OC3CCN(C)CC3)cn2)cc1Nc1cccc(-c2ncn(C)n2)c1OC] | 1H NMR (300 MHz, DMSO-d6) δ 11.10 (s, 1H), 9.81 (s, 1H), 8.82 (m, 1H), 8.57 (m, 1H), 7.90 (m, 1H), 7.75 (m, 1H), 7.68-7.57 (m, 3H), 7.43-7.29 (m, 2H), 4.30 (m, 1H), 3.96 (s, 3H), 3.75 (s, 3H), 3.07 (m, 2H), 2.62 (m, 2H), 2.22-2.10 (m, 5H), 1.90 (m, 2H), 1.62 (m, 2H), 1.15 (m, 3H). | 543.25 |
| 18Z | 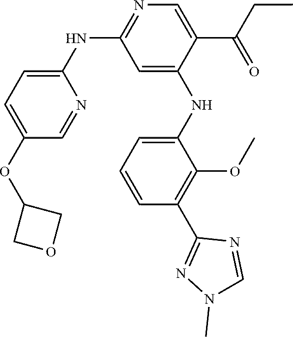 [SMILES: CCC(=O)c1cnc(Nc2ccc(OC3COC3)cn2)cc1Nc1cccc(-c2ncn(C)n2)c1OC] | 1H NMR (400 MHz, Methanol-d4) δ 8.77 (s, 1H), 8.51 (s, 1H), 7.78 - 7.75 (m, 1H), 7.74 - 7.62 (m, 2H), 7.62 (s, 1H), 7.44 - 7.37 (m, 1H), 7.32 (t, J = 7.9 Hz, 1H), 7.29 - 7.15 (m, 1H), 5.39 - 5.24 (m, 1H), 5.12 - 4.98 (m, 2H), 4.74 - 4.65 (m, 2H), 4.04 (s, 3H), 3.74 (s, 3H), 3.07 (q, J = 7.3 Hz, 2H), 1.25 (t, J = 7.3 Hz, 3H). | 502.2 |
| 18AA | 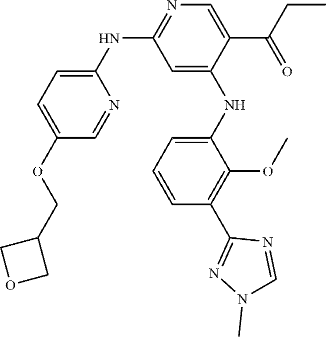 [SMILES: CCC(=O)c1cnc(Nc2ccc(OCC3COC3)cn2)cc1Nc1cccc(-c2ncn(C)n2)c1OC] | 1H NMR (300 MHz, Methanol-d4) δ 8.76 (s, 1H), 8.50 (s, 1H), 7.94 (t, J = 1.8 Hz, 1H), 7.82 - 7.75 (m, 2H), 7.61 (s, 1H), 7.42 - 7.35 (m, 2H), 7.32 (t, J = 7.9 Hz, 1H), 4.89 (d, J = 14.1 Hz, 1H), 4.86 - 4.84 (m, 1H), 4.62 (t, J = 6.1 Hz, 2H), 4.25 (d, J = 6.3 Hz, 2H), 4.04 (s, 3H), 3.74 (s, 3H), 3.48 - 3.38 (m, 1H), 3.06 (q, J = 7.3 Hz, 2H), 1.25 (t, J = 7.3 Hz, 3H). | 516.3 |
| 18BB | 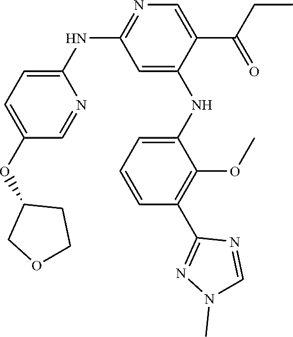 [SMILES: CCC(=O)c1cnc(Nc2ccc(O[C@@H]3CCOC3)cn2)cc1Nc1cccc(-c2ncn(C)n2)c1OC] | 1H NMR (400 MHz, Methanol-d4) δ 8.77 (s, 1H), 8.50 (s, 1H), 7.98 - 7.79 (m, 1H), 7.78 - 7.70 (m, 1H), 7.68 -7.62 (m, 1H), 7.61 (s, 1H), 7.43 -7.28 (m, 3H), 5.09 - 4.93 (m, 1H), 4.04 (s, 3H), 4.01 - 3.85 (m, 4H), 3.74 (s, 3H), 3.07 (q, J = 7.3 Hz, 2H), 2.33 - 2.19 (m, 1H), 2.15-2.02 (m, 1H), 1.25 (t, J = 7.3 Hz, 3H). | 516.3 |
| 18CC | 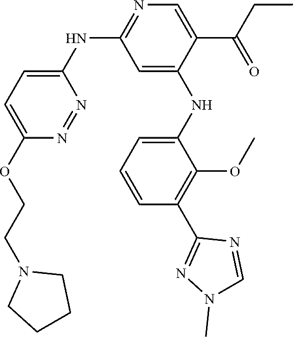 [SMILES: CCC(=O)c1cnc(Nc2ccc(OCCN3CCCC3)nn2)cc1Nc1cccc(-c2ncn(C)n2)c1OC] | 1H NMR (300 MHz, Methanol-d4) δ 8.78 (m, 1H), 8.48 (m, 1H), 8.00 (m, 1H), 7.68-7.61 (m, 2H), 7.35 (m, 1H), 7.29 (m, 1H), 7.10 (m, 1H), 4.53 (m, 2H), 4.02 (s, 3H), 3.72 (s, 3H), 3.08 (m, 2H), 2.98 (m, 2H), 2.69 (m, 4H), 1.86 (m, 4H), 1.24 (m, 3H). | 544.25 |
| 18DD | 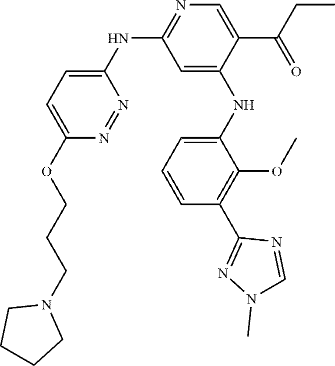 [SMILES: CCC(=O)c1cnc(Nc2ccc(OCCCN3CCCC3)nn2)cc1Nc1cccc(-c2ncn(C)n2)c1OC] | 1H NMR (400 MHz, Methanol-d4) δ 8.81 (m, 1H), 8.48 (m, 1H), 8.00 (m, 1H), 7.66 (m, 2H), 7.32 (m, 2H), 7.10 (m, 1H), 4.43 (m, 2H), 4.03 (s, 3H), 3.71 (s, 3H), 3.08 (m, 2H), 2.72 (m, 2H), 2.65 (m, 4H), 2.08 (m, 2H), 1.85 (m, 4H), 1.24 (m, 3H). | 558.4 |
| 18EE | 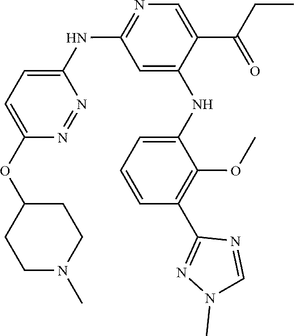 [SMILES: CCC(=O)c1cnc(Nc2ccc(OC3CCN(C)CC3)nn2)cc1Nc1cccc(-c2ncn(C)n2)c1OC] | 1H NMR (400 MHz, Methanol-d4) δ 8.79 (m, 1H), 8.48 (m, 1H), 8.00 (m, 1H), 7.65 (m, 2H), 7.28 (m, 2H), 7.05 (m, 1H), 5.12 (m, 1H), 4.02 (s, 3H), 3.69 (s, 3H), 3.05 (m, 2H), 2.78 (m, 2H), 2.41 (m, 2H), 2.32 (s, 3H), 2.13 (m, 2H), 1.88 (m, 2H), 1.24 (m, 3H). | 544.30 |
| 18FF | 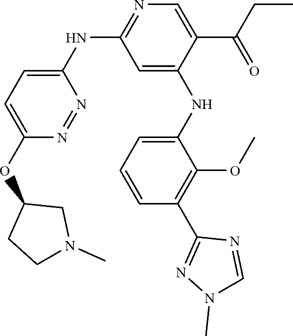 [SMILES: CCC(=O)c1cnc(Nc2ccc(O[C@@H]3CCN(C)C3)nn2)cc1Nc1cccc(-c2ncn(C)n2)c1OC] | 1H NMR (400 MHz, Methanol-d4) δ 8.79 (m, 1H), 8.48 (m, 1H), 8.02 (m, 1H), 7.65 (m, 2H), 7.29 (m, 2H), 7.08 (m, 1H), 5.50 (m, 1H), 4.02 (s, 3H), 3.71 (s, 3H), 3.10 (m, 2H), 2.93 (m, 3H), 2.58 (m, 2H), 2.44 (s, 3H), 2.03 (m, 1H), 1.24 (m, 3H). | 528.20 [M-H] |
| 18GG | 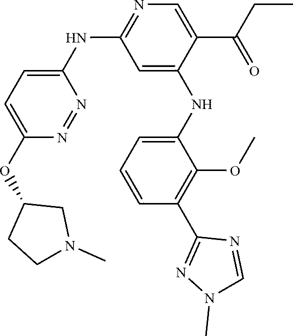 [SMILES: CCC(=O)c1cnc(Nc2ccc(O[C@H]3CCN(C)C3)nn2)cc1Nc1cccc(-c2ncn(C)n2)c1OC] | 1H NMR (300 MHz, DMSO-d6) δ 11.10 (s, 1H), 10.08 (s, 1H), 8.85 (m, 1H), 8.58 (m, 1H), 7.98 (m, 1H), 7.64 (m, 3H), 7.29 (m, 1H), 7.16 (m, 1H), 5.43 (m, 1H), 3.97 (s, 3H), 3.77 (s, 3H), 3.09 (m, 2H), 2.8 (m, 1H), 2.7 (m, 2H), 2.35 (m, 2H), 2.28 (s, 3H), 1.83 (m, 1H), 1.14 (m, 3H). | 530.30 |
| 18HH | 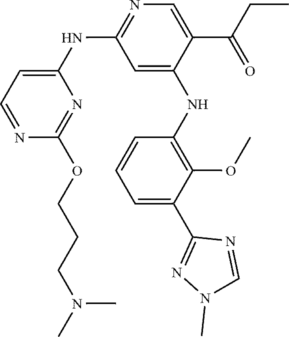 [SMILES: CCC(=O)c1cnc(Nc2ccnc(OCCCN(C)C)n2)cc1Nc1cccc(-c2ncn(C)n2)c1OC] | 1H NMR (400 MHz, Methanol-d4) δ 8.86 (s, 1H), 8.50 (s, 1H), 8.13 (d, J = 5.8 Hz, 1H), 7.93 (s, 1H), 7.75 (dd, J = 7.8, 1.6 Hz, 1H), 7.65 (dd, J = 8.0, 1.6 Hz, 1H), 7.32 (t, J = 7.9 Hz, 1H), 6.95 (d, J = 5.9 Hz, 1H), 4.03 (s, 3H), 3.94 (t, J = 6.0 Hz, 2H), 3.76 (s, 3H), 3.11 (q, J = 7.3 Hz, 2H), 2.43 - 2.34 (m, 2H), 2.20 (s, 6H), 1.82 - 1.70 (m, 2H), 1.26 (t, J = 7.3 Hz, 3H). | |
| 1811 | 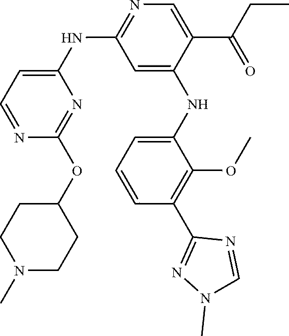 [SMILES: CCC(=O)c1cnc(Nc2ccnc(OC3CCN(C)CC3)n2)cc1Nc1cccc(-c2ncn(C)n2)c1OC] | 1H NMR (300 MHz, DMSO-d6) δ 11.18 (s, 1H), 10.33 (s, 1H), 8.88 (m, 1H), 8.55 (m, 1H), 8.19 (m, 1H), 8.00 (m, 1H), 7.64 (m, 2H), 7.25 (m, 1H), 7.09 (m, 1H), 4.74 (m, 1H), 3.94 (s, 3H), 3.76 (s, 3H), 3.11 (m, 2H), 2.55 (m, 2H), 2.12-2.00 (m, 5H), 1.84 (m, 2H), 1.62 (m, 2H), 1.14 (m, 3H). | 544.35 |
| 18JJ | 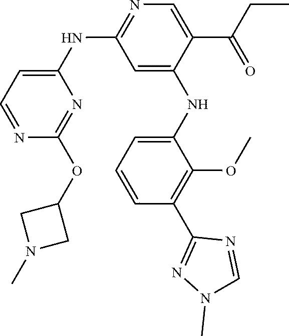 [SMILES: CCC(=O)c1cnc(Nc2ccnc(OC3CN(C)C3)n2)cc1Nc1cccc(-c2ncn(C)n2)c1OC] | 1H NMR (400 MHz, Methanol-d4) δ 8.88 (m, 1H), 8.51 (m, 1H), 8.17 (m, 1H), 7.76-7.70 (m, 3H), 7.36 (m, 1H), 7.21 (m, 1H), 4.96 (m, 1H), 4.04 (s, 3H), 3.77 (s, 3H), 3.64 (m, 2H), 3.33 (m, 2H), 3.13 (m, 2H), 2.34 (s, 3H), 1.27 (m, 3H). | 516.25 |
| 18KK | 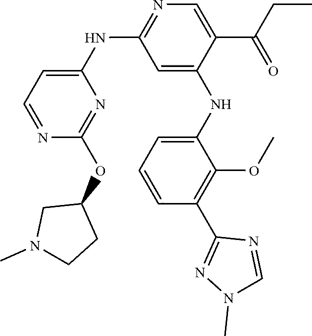 [SMILES: CCC(=O)c1cnc(Nc2ccnc(O[C@H]3CCN(C)C3)n2)cc1Nc1cccc(-c2ncn(C)n2)c1OC] | 1H NMR (300 MHz, DMSO-d6) δ 11.15 (s, 1H), 10.37 (s, 1H), 8.90 (m, 1H), 8.57 (m, 1H), 8.19 (m, 1H), 8.12 (m, 1H), 7.67 (m, 2H), 7.34 (m, 1H), 6.99 (m, 1H), 4.98 (m, 1H), 3.95 (s, 3H), 3.76 (s, 3H), 3.11 (m, 2H), 2.64 (m, 2H), 2.45 (m, 1H), 2.2-2.08 (m, 5H), 1.72 (m, 1H), 1.15 (m, 3H). | 530.25 |
| 18LL | 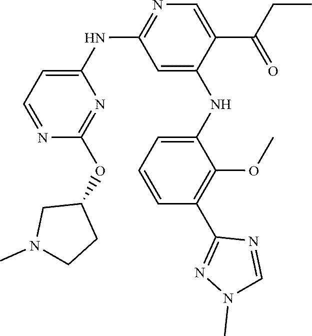 [SMILES: CCC(=O)c1cnc(Nc2ccnc(O[C@@H]3CCN(C)C3)n2)cc1Nc1cccc(-c2ncn(C)n2)c1OC] | 1H NMR (300 MHz, DMSO-d6) δ 11.15 (s, 1H), 10.36 (s, 1H), 8.90 (m, 1H), 8.57 (m, 1H), 8.20 (m, 1H), 8.12 (m, 1H), 7.67 (m, 2H), 7.35 (m, 1H), 7.00 (m, 1H), 4.98 (m, 1H), 3.96 (s, 3H), 3.77 (s, 3H), 3.12 (m, 2H), 2.64 (m, 2H), 2.52 (m, 1H), 2.40 (m, 1H) 2.2-2.05 (m, 4H), 1.74 (m, 1H), 1.14 (m, 3H). | 530.75 |
| 18MM | 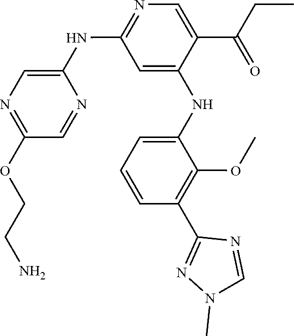 [SMILES: CCC(=O)c1cnc(Nc2cnc(OCCN)cn2)cc1Nc1cccc(-c2ncn(C)n2)c1OC] | 1H NMR (400 MHz, Methanol-d4) δ 8.73 (m, 1H), 8.49 (s, 1H), 8.22 (m, 1H), 7.72 (m, 1H), 7.63 (m, 2H), 7.28 (m, 1H), 7.08 (m, 1H), 4.03 (s, 3H), 3.74 (m, 5H), 3.45 (m, 2H), 3.04 (m, 2H), 1.24 (m, 3H). | 490.15 |
| 18NN | 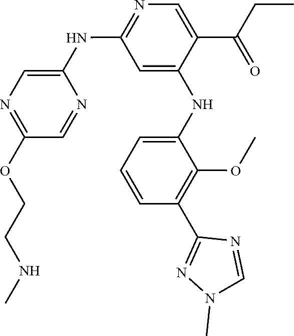 [SMILES: CCC(=O)c1cnc(Nc2cnc(OCCNC)cn2)cc1Nc1cccc(-c2ncn(C)n2)c1OC] | 1H NMR (400 MHz, Methanol-d4) δ 8.78 (m, 1H), 8.51 (m, 2H), 7.90 (m, 1H), 7.65 (m, 2H), 7.30 (m, 2H), 4.39 (m, 2H), 4.04 (s, 3H), 3.73 (s, 3H), 3.05 (m, 2H), 2.97 (m, 2H), 2.47 (s, 3H), 1.24 (m, 3H). | 504.15 |
| 18OO | 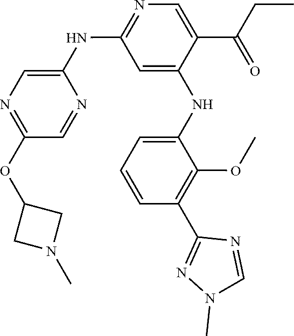 [SMILES: CCC(=O)c1cnc(Nc2cnc(OC3CN(C)C3)cn2)cc1Nc1cccc(-c2ncn(C)n2)c1OC] | 1H NMR (300 MHz, DMSO-d6) δ 11.03 (s, 1H), 9.93 (s, 1H), 8.85 (m, 1H), 8.70 (m, 1H), 8.56 (m, 1H), 7.97 (m, 1H), 7.61 (m, 2H), 7.30 (m, 3H), 5.04 (m, 1H), 3.95 (s, 3H), 3.75-3.68 (m, 5H), 3.12 (m, 2H), 3.00 (m, 2H), 2.31 (s, 3H), 1.15 (m, 3H). | 514.15 |
| 18PP | 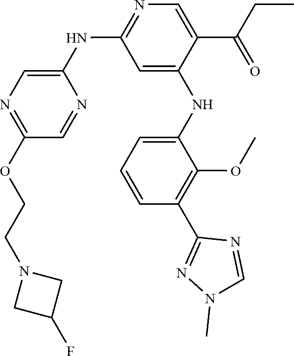 [SMILES: CCC(=O)c1cnc(Nc2cnc(OCCN3CC(F)C3)cn2)cc1Nc1cccc(-c2ncn(C)n2)c1OC] | 1H NMR (300 MHz, Methanol-d4) δ 8.81 (m, 1H), 8.54 (m, 2H), 7.96 (m, 1H), 7.67 (m, 2H), 7.32 (m, 1H), 7.21 (m, 1H), 5.2 (m, 1H), 4.6 (m, 2H), 4.32 (m, 2H), 4.1-4.0 (m, 5H), 3.73 (s, 3H), 3.45 (m, 2H), 3.09 (m, 2H), 1.28 (m, 3H). | 548.20 |
| 18QQ | 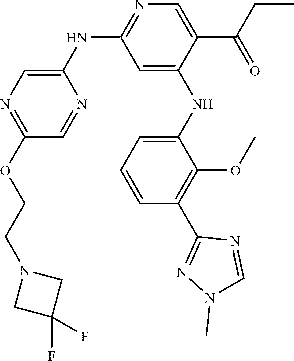 [SMILES: CCC(=O)c1cnc(Nc2cnc(OCCN3CC(F)(F)C3)cn2)cc1Nc1cccc(-c2ncn(C)n2)c1OC] | 1H NMR (300 MHz, Methanol-d4) δ 8.84 (m, 1H), 8.55 (m, 1H), 8.11 (m, 1H), 8.00 (m, 1H), 7.88 (m, 1H), 7.59 (m, 1H), 7.37 (m, 1H), 6.54 (m, 1H), 4.85 (m, 4H), 4.64 (m, 2H), 4.03 (s, 3H), 3.82 (m, 2H), 3.71 (s, 3H), 3.13 (m, 2H), 1.26 (m, 3H). | 566.15 |
| 18RR | 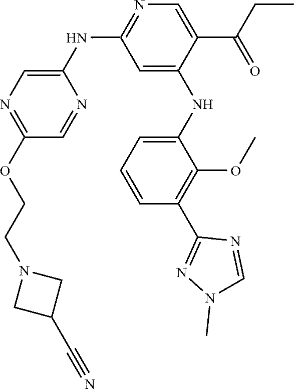 [SMILES: CCC(=O)c1cnc(Nc2cnc(OCCN3CC(C#N)C3)cn2)cc1Nc1cccc(-c2ncn(C)n2)c1OC] | 1H NMR (300 MHz, Methanol-d4) δ 8.85 (m, 1H), 8.56 (m, 1H), 8.13 (m, 1H), 8.02 (m, 1H), 7.89 (m, 1H), 7.60 (m, 1H), 7.42 (m, 1H), 6.53 (m, 1H), 4.62 (m, 6H), 4.05 (s, 3H), 3.78 (m, 2H), 3.73 (s, 3H), 3.12 (m, 2H), 1.29 (m, 3H). | 555.20 |
| 18SS | 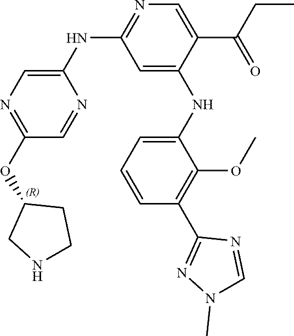 [SMILES: CCC(=O)c1cnc(Nc2cnc(O[C@@H]3CCNC3)cn2)cc1Nc1cccc(-c2ncn(C)n2)c1OC] | 1H NMR (400 MHz, DMSO-d6) δ 11.02 (s, 1H), 9.96 (s, 1H), 8.83 (m, 1H), 8.73 (m, 1H), 8.55 (m, 1H), 7.94 (m, 1H), 7.60 (m, 2H), 7.28 (m, 2H), 5.41 (m, 1H), 3.94 (s, 3H), 3.72 (s, 3H), 3.38 (m, 1H), 3.21-3.03 (m, 5H), 2.22-1.97 (m, 2H), 1.12 (m, 3H). | 516.20 |
| 18TT | 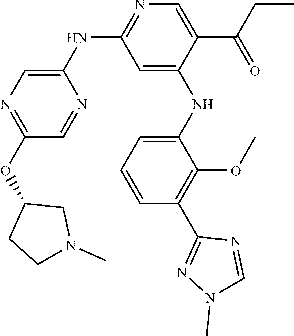 [SMILES: CCC(=O)c1cnc(Nc2cnc(O[C@H]3CCN(C)C3)cn2)cc1Nc1cccc(-c2ncn(C)n2)c1OC] | 1H NMR (400 MHz, DMSO-d6) δ 11.03 (s, 1H), 9.91 (s, 1H), 8.85 (m, 1H), 8.71 (m, 1H), 8.56 (m, 1H), 7.94 (m, 1H), 7.61 (m, 2H), 7.31 (m, 2H), 5.26 (m, 1H), 3.95 (s, 3H), 3.74 (s, 3H), 3.10 (m, 2H), 2.8-2.65 (m, 3H), 2.45-2.28 (m, 2H), 2.26 (s, 3H), 1.81 (m, 1H), 1.15 (m, 3H). | 530.35 |
| 18UU | 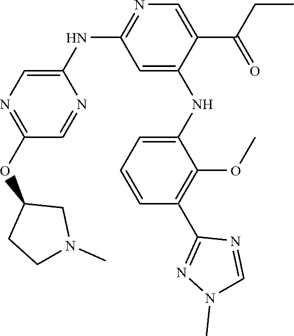 [SMILES: CCC(=O)c1cnc(Nc2cnc(O[C@@H]3CCN(C)C3)cn2)cc1Nc1cccc(-c2ncn(C)n2)c1OC] | 1H NMR (400 MHz, D₂O) δ 9.19 (s, 1H), 8.65 (s, 1H), 7.99 (m, 1H), 7.90 (m, 1H), 7.68 (m, 1H), 7.54 (m, 1H), 7.31 (m, 1H), 6.44 (m, 1H), 5.60-5.48 (m, 1H), 4.05 (s, 3H), 3.82 (m, 2H), 3.57 (s, 3H), 3.40 (m, 1H), 3.24 (m, 1H), 3.02-2.95 (m, 5H), 2.68 (m, 1H), 2.4-2.25 (m, 1H), 1.06 (m, 3H). | 530.05 |
| 18VV | 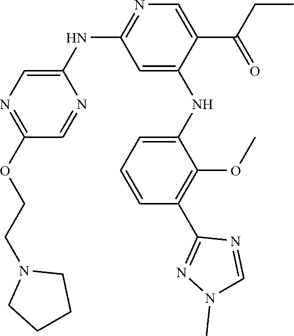 [SMILES: CCC(=O)c1cnc(Nc2cnc(OCCN3CCCC3)cn2)cc1Nc1cccc(-c2ncn(C)n2)c1OC] | 1H NMR (300 MHz, Methanol-d4) δ 8.76 (m, 1H), 8.48 (m, 2H), 7.87 (m, 1H), 7.64 (m, 2H), 7.32-7.25 (m, 2H), 4.44 (m, 2H), 4.02 (s, 3H), 3.71 (s, 3H), 3.06 (m, 2H), 2.92 (m, 2H), 2.68 (m, 4H), 1.84 (m, 4H), 1.22 (m, 3H). | 544.20 |
| 18WW | 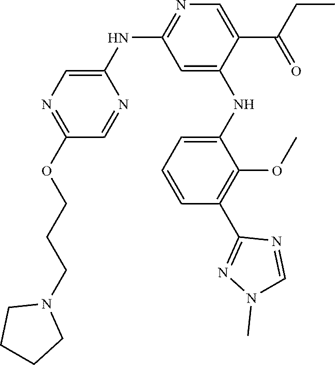 [SMILES: CCC(=O)c1cnc(Nc2cnc(OCCCN3CCCC3)cn2)cc1Nc1cccc(-c2ncn(C)n2)c1OC] | 1H NMR (300 MHz, Methanol-d4) δ 8.81 (m, 1H), 8.52 (m, 2H), 7.90 (m, 1H), 7.70-7.64 (m, 2H), 7.32 (m, 2H), 4.37 (m, 2H), 4.04 (s, 3H), 3.73 (s, 3H), 3.10 (m, 2H), 2.82-2.78 (m, 6H), 2.08 (m, 2H), 1.89 (m, 4H), 1.28 (m, 3H). | 558.30 |
| 18XX | 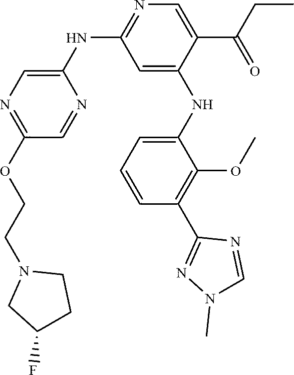 [SMILES: CCC(=O)c1cnc(Nc2cnc(OCCN3CC[C@H](F)C3)cn2)cc1Nc1cccc(-c2ncn(C)n2)c1OC] | 1H NMR (300 MHz, Methanol-d4) δ 8.82 (m, 1H), 8.53 (m, 2H), 7.91 (m, 1H), 7.67 (m, 2H), 7.31 (m, 1H) 5.22 (m, 1H), 4.47 (m, 2H), 4.04 (s, 3H), 3.73 (s, 3H), 3.15-2.6 (m, 8H), 2.32-1.92 (m, 2H), 1.26 (m, 3H). | 562.25 |
| 18YY | 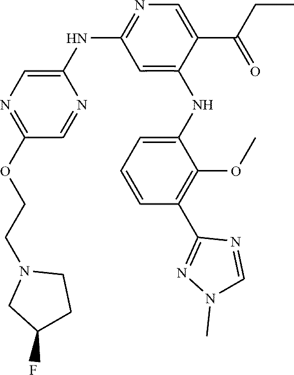 [SMILES: CCC(=O)c1cnc(Nc2cnc(OCCN3CC[C@@H](F)C3)cn2)cc1Nc1cccc(-c2ncn(C)n2)c1OC] | 1H NMR (300 MHz, Methanol-d4) δ 8.81 (m, 1H), 8.52 (m, 2H), 7.90 (m, 1H), 7.68 (m, 2H), 7.32 (m, 1H) 5.2 (m, 1H), 4.47 (m, 2H), 4.04 (s, 3H), 3.73 (s, 3H), 3.15-2.6 (m, 8H), 2.32-1.92 (m, 2H), 1.26 (m, 3H). | 562.35 |
| 18ZZ | 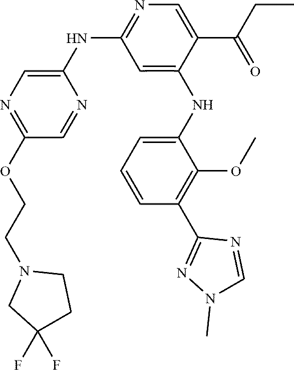 [SMILES: CCC(=O)c1cnc(Nc2cnc(OCCN3CCC(F)(F)C3)cn2)cc1Nc1cccc(-c2ncn(C)n2)c1OC] | 1H NMR (300 MHz, Methanol-d4) δ 8.86 (m, 1H), 8.56 (m, 1H), 8.15 (m, 1H), 8.02 (m, 1H), 7.90 (m, 1H), 7.59 (m, 1H), 7.41 (m, 1H), 6.54 (m, 1H), 4.71 (m, 2H), 4.05-3.97 (m, 5H), 3.85-3.75 (m, 7H), 3.17 (m, 2H), 2.82 (m, 2H), 1.30 (m, 3H). | 580.20 |
| 18AAA | 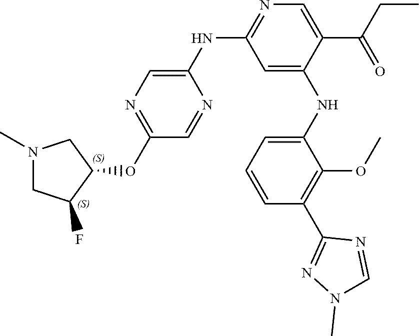 [SMILES: CCC(=O)c1cnc(Nc2cnc(O[C@H]3CN(C)C[C@@H]3F)cn2)cc1Nc1cccc(-c2ncn(C)n2)c1OC] | 1H NMR (300 MHz, Methanol-d4) δ 8.87 (m, 1H), 8.57 (m, 1H), 8.14 (m, 1H), 8.05 (m, 1H), 7.88 (m, 1H), 7.59 (m, 1H), 7.41 (m, 1H), 5.75 (m, 1H), 5.62-5.46 (m, 1H), 4.05 (s, 3H), 4.0-3.8 (m, 4H), 3.73 (s, 3H), 3.2-3.13 (m, 5H), 1.28 (m, 3H). | 548.00 |
| 18BBB | 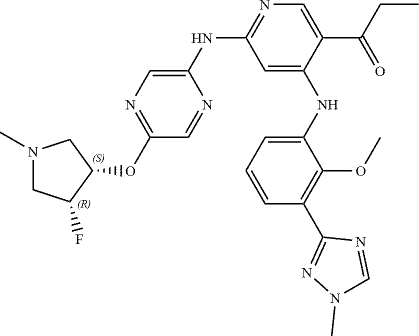 [SMILES: CCC(=O)c1cnc(Nc2cnc(O[C@H]3CN(C)C[C@H]3F)cn2)cc1Nc1cccc(-c2ncn(C)n2)c1OC] | 1H NMR (300 MHz, Methanol-d4) δ 8.79 (m, 1H), 8.53 (m, 2H), 7.92 (m, 1H), 7.65 (m, 2H), 7.30 (m, 2H), 5.42-5.23 (m, 2H), 4.04 (s, 3H), 3.73 (s, 3H), 3.12-3.01 (m, 6H), 2.47 (s, 3H), 1.28 (m, 3H). | 548.20 |
| 18CCC | 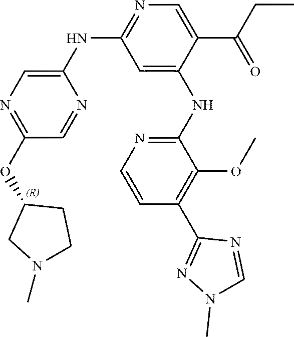 [SMILES: CCC(=O)c1cnc(Nc2cnc(O[C@@H]3CCN(C)C3)cn2)cc1Nc1nccc(-c2ncn(C)n2)c1OC] | 1H NMR (400 MHz, DMSO-d6) δ 12.15 (s, 1H), 10.10 (s, 1H), 9.02 (m, 1H), 8.90 (m, 1H), 8.78 (m, 1H), 8.55 (m, 2H), 8.04 (m, 1H), 7.77 (m, 1H), 5.30 (m, 1H), 4.05 (s, 3H), 3.95 (s, 3H), 3.10 (m, 2H), 2.78-2.62 (m, 3H), 2.38-2.25 (5H), 1.82 (m, 1H), 1.14 (m, 3H). | 531.40 |
| 18DDD | 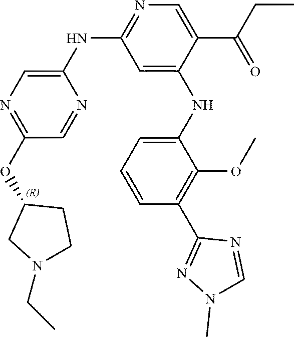 [SMILES: CCC(=O)c1cnc(Nc2cnc(O[C@@H]3CCN(CC)C3)cn2)cc1Nc1cccc(-c2ncn(C)n2)c1OC] | 1H NMR (400 MHz, Methanol-d4) δ 8.83 (m, 1H), 8.53 (m, 1H), 8.09 (m, 1H), 8.00 (m, 1H), 7.86 (m, 2H), 7.57 (m, 1H), 7.37 (m, 1H), 6.53 (m, 1H), 5.67 (m, 1H), 4.03 (s, 3H), 3.8 (m, 2H), 3.71 (s, 3H), 3.42-3.3 (m, 4H), 3.12 (m, 2H), 2.7 (m, 1H), 2.48-2.27 (m, 1H), 1.37 (m, 3H), 1.26 (m, 3H). | 544.30 |
| 18EEE | 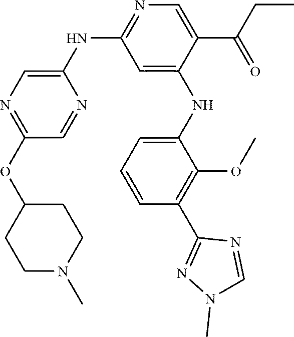 [SMILES: CCC(=O)c1cnc(Nc2cnc(OC3CCN(C)CC3)cn2)cc1Nc1cccc(-c2ncn(C)n2)c1OC] | 1H NMR (400 MHz, Methanol-d4) δ 8.78 (m, 1H), 8.52-8.46 (m, 4H), 7.90 (m, 1H), 7.64 (m, 2H), 7.29 (m, 2H), 5.21 (m, 1H), 4.02 (s, 3H), 3.71 (s, 3H), 3.35 (m, 2H), 3.25 (m, 2H), 3.05 (m, 2H), 2.84 (s, 3H), 2.15 (m, 4H), 1.24 (m, 3H). | 544.30 |
| 18FFF | 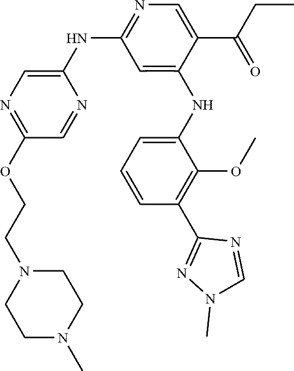 [SMILES: CCC(=O)c1cnc(Nc2cnc(OCCN3CCN(C)CC3)cn2)cc1Nc1cccc(-c2ncn(C)n2)c1OC] | 1H NMR (400 MHz, DMSO-d6) δ 11.04 (s, 1H), 9.93 (s, 1H), 8.85 (m, 1H), 8.72 (m, 1H), 8.57 (m, 1H), 7.97 (m, 1H), 7.62 (m, 2H), 7.32 (m, 2H), 4.34 (m, 2H), 3.95 (s, 3H), 3.74 (s, 3H), 3.10 (m, 2H), 2.69 (m, 2H), 2.5-2.25 (m, 8H), 2.14 (s, 3H), 1.15 (m, 3H). | 571.30 |
| 18GGG | 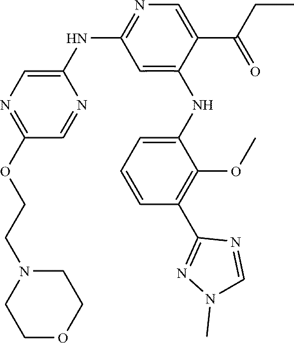 [SMILES: CCC(=O)c1cnc(Nc2cnc(OCCN3CCOCC3)cn2)cc1Nc1cccc(-c2ncn(C)n2)c1OC] | 1H NMR (300 MHz, Methanol-d4) δ 8.80 (m, 1H), 8.52 (m, 2H), 7.89 (m, 1H), 7.65 (m, 2H), 7.31 (m, 2H), 4.47 (m, 2H), 4.04 (s, 3H), 3.74-3.68 (m, 7H), 3.09 (m, 2H), 2.82 (m, 2H), 2.62 (m, 4H), 1.25 (m, 3H). | 560.30 |
| 18HHH | 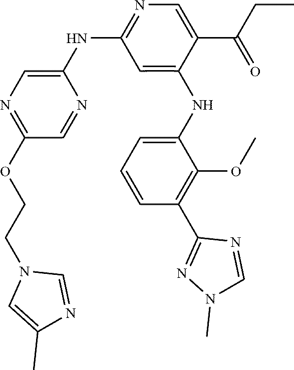 [SMILES: CCC(=O)c1cnc(Nc2cnc(OCCn3cnc(C)c3)cn2)cc1Nc1cccc(-c2ncn(C)n2)c1OC] | 1H NMR (400 MHz, Methanol-d4) δ 8.99-8.90 (m, 3H), 8.12 (m, 1H), 8.02 (m, 1H), 7.89 (m, 1H), 7.65 (m, 1H), 7.45 (m, 2H), 6.60 (m, 1H), 4.72 (m, 2H), 4.66 (m, 2H), 4.10 (s, 3H), 3.75 (s, 3H), 3.12 (m, 2H), 2.36 (s, 3H), 1.26 (m, 3H). | 555.20 |
| 18III | 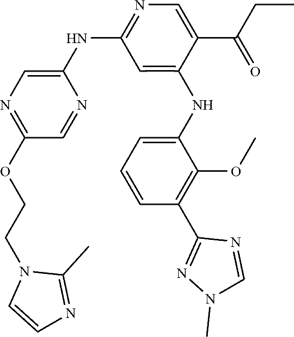 [SMILES: CCC(=O)c1cnc(Nc2cnc(OCCn3ccnc3C)cn2)cc1Nc1cccc(-c2ncn(C)n2)c1OC] | 1H NMR (400 MHz, Methanol-d4) δ 8.80 (m, 1H), 8.52 (m, 2H), 7.88 (m, 1H), 7.66 (m, 2H), 7.32 (m, 2H), 7.08 (m, 1H), 6.80 (m, 1H), 4.59 (m, 2H), 4.37 (m, 2H), 4.04 (s, 3H), 3.73 (s, 3H), 3.09 (m, 2H), 2.42 (s, 3H), 1.25 (m, 3H). | 555.20 |
| 18JJJ | 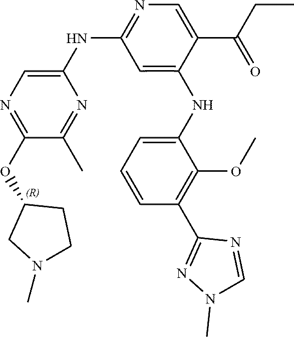 [SMILES: CCC(=O)c1cnc(Nc2cnc(O[C@@H]3CCN(C)C3)c(C)n2)cc1Nc1cccc(-c2ncn(C)n2)c1OC] | 1H NMR (300 MHz, Methanol-d4) δ 8.78 (m, 1H), 8.49 (m, 1H), 8.09 (m, 1H), 7.72-7.64 (m, 3H), 7.32 (m, 1H), 5.41 (m, 1H), 4.04 (s, 3H), 3.8 (m, 2H), 3.74 (s, 3H), 3.08 (m, 2H), 2.98-2.85 (m, 3H), 2.54 (m, 1H), 2.48-2.34 (m, 6H), 2.03 (m, 1H), 1.28 (m, 3H). | 544.35 |
| 18KKK | 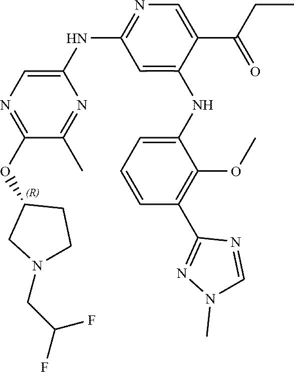 [SMILES: CCC(=O)c1cnc(Nc2cnc(O[C@@H]3CCN(CC(F)F)C3)c(C)n2)cc1Nc1cccc(-c2ncn(C)n2)c1OC] | 1H NMR (400 MHz, DMSO-d6) δ 11.09 (s, 1H), 10.72 (s, 1H), 8.89 (m, 1H), 8.59 (m, 1H), 8.45 (m, 1H), 8.03 (m, 1H), 7.74 (m, 1H), 7.59 (m, 1H), 7.36 (m, 1H), 7.04 (m, 1H), 6.46 (m, 1H), 5.51 (m, 1H), 3.96 (s, 3H), 3.85-3.76 (m, 6H), 3.65-3.42 (m, 3H), 3.12 (m, 2H), 2.52 (m, 1H), 2.20 (m, 1H), 1.16 (m, 3H). | 580.20 |
| 18LLL | 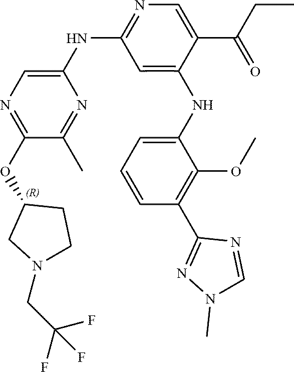 [SMILES: CCC(=O)c1cnc(Nc2cnc(O[C@@H]3CCN(CC(F)(F)F)C3)c(C)n2)cc1Nc1cccc(-c2ncn(C)n2)c1OC] | 1H NMR (400 MHz, DMSO-d6) δ 11.11 (s, 1H), 11.07 (s, 1H), 8.98 (m, 1H), 8.60 (m, 1H), 8.22 (m, 1H), 8.05 (m, 1H), 7.80 (m, 1H), 7.57 (m, 1H), 7.37 (m, 1H), 6.78 (m, 1H), 5.36 (m, 1H), 3.96 (s, 3H), 3.74 (s, 3H), 3.51 (m, 2H), 3.25-3.02 (m, 4H), 2.82 (m, 1H), 2.34 (m, 1H), 1.96 (m, 1H), 1.15 (m, 3H). | 598.20 |
| 18MMM | 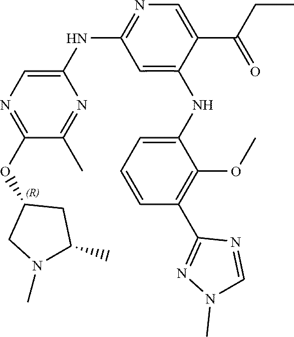 [SMILES: CCC(=O)c1cnc(Nc2cnc(O[C@@H]3C[C@H](C)N(C)C3)c(C)n2)cc1Nc1cccc(-c2ncn(C)n2)c1OC] | 1H NMR (400 MHz, Methanol-d4) δ 8.80 (m, 1H), 8.51 (m, 2H), 7.88 (m, 1H), 7.66 (m, 2H), 7.3 (m, 2H), 5.31 (m, 1H), 4.04 (s, 3H), 3.73 (s, 3H), 3.28 (m, 1H), 3.09 (m, 2H), 2.62 (m, 2H), 2.32 (m, 4H), 1.65 (m, 1H), 1.25 (m, 6H). | 544.20 |
| 18NNN | 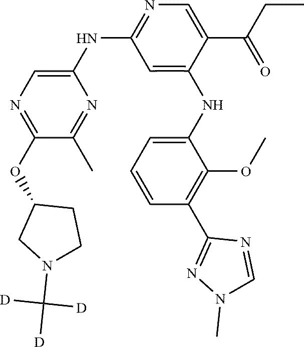 [SMILES: [2H]C([2H])([2H])N1CC[C@@H](Oc2ncc(Nc3cc(Nc4cccc(-c5ncn(C)n5)c4OC)c(C(=O)CC)cn3)nc2C)C1] | 1H NMR (300 MHz, Methanol-d4) δ 8.79 (m, 1H), 8.51 (m, 2H), 7.87 (m, 1H), 7.65 (m, 2H), 7.30 (m, 2H), 5.39 (m, 1H), 4.04 (s, 3H), 3.73 (s, 3H), 3.07 (m, 2H), 2.92-2.85 (m, 3H), 2.58-2.39 (m, 2H), 2.01 (m, 1H), 1.26 (m, 3H). | 533.30 |
| 18OOO | 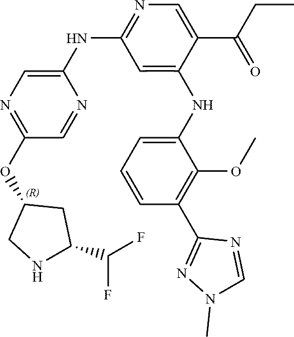 [SMILES: CCC(=O)c1cnc(Nc2cnc(O[C@H]3CN[C@@H](C(F)F)C3)cn2)cc1Nc1cccc(-c2ncn(C)n2)c1OC] | 1H NMR (400 MHz, Methanol-d4) δ 8.81 (m, 1H), 8.51 (m, 2H), 7.91 (m, 1H), 7.66 (m, 2H), 7.32 (m, 1H), 7.25 (m, 1H), 5.94 (m, 1H), 5.48 (m, 1H), 4.04 (s, 3H), 3.73 (s, 3H), 3.68 (m, 1H), 3.34 (m, 1H), 3.25 (m, 1H), 3.09 (m, 2H), 2.51 (m, 1H), 2.08 (m, 1H), 1.25 (m, 3H). | 566.30 |
| 18PPP | 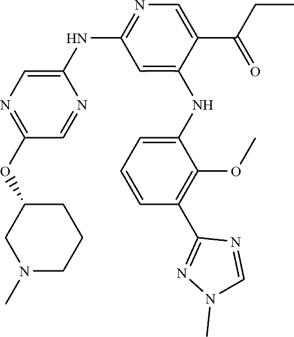 [SMILES: CCC(=O)c1cnc(Nc2cnc(O[C@@H]3CCCN(C)C3)cn2)cc1Nc1cccc(-c2ncn(C)n2)c1OC] | 1H NMR (400 MHz, DMSO-d6) δ 11.10 (s, 1H), 9.93 (s, 1H), 8.87 (m, 1H), 8.71 (m, 1H), 8.57 (m, 1H), 7.94 (m, 1H), 7.62 (m, 2H), 7.31 (m, 2H), 4.95 (m, 1H), 3.96 (s, 3H), 3.74 (s, 3H), 3.10 (m, 2H), 2.90 (m, 1H), 2.65 (m, 1H), 2.25-2.10 (m, 5H), 1.98 (m, 1H), 1.77 (m, 1H), 1.6-1.35 (m, 2H), 1.15 (m, 3H). | 544.20 |
| 18QQQ | 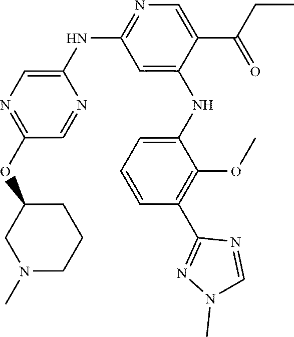 [SMILES: CCC(=O)c1cnc(Nc2cnc(O[C@H]3CCCN(C)C3)cn2)cc1Nc1cccc(-c2ncn(C)n2)c1OC] | 1H NMR (400 MHz, Methanol-d4) δ 8.83 (m, 1H), 8.50 (m, 2H), 7.88 (m, 1H), 7.69 (m, 2H), 7.32 (m, 2H), 5.10 (m, 1H), 4.04 (s, 3H), 3.73 (s, 3H), 3.09 (m, 2H), 2.89 (m, 1H), 2.60 (m, 1H), 2.45-2.30 (m, 5H), 1.95 (m, 2H), 1.66 (m, 2H), 1.26 (m, 3H). | 544.30 |
| 18RRR | 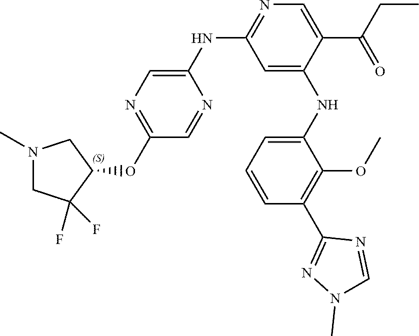 [SMILES: CCC(=O)c1cnc(Nc2cnc(O[C@H]3CN(C)CC3(F)F)cn2)cc1Nc1cccc(-c2ncn(C)n2)c1OC] | 1H NMR (400 MHz, Methanol-d4) δ 8.81 (m, 1H), 8.56 (m, 1H), 8.50 (m, 1H), 7.95 (m, 1H), 7.66 (m, 2H), 7.31 (m, 2H), 5.47 (m, 1H), 4.04 (s, 3H), 3.73 (s, 3H), 3.32 (m, 1H), 3.15-2.9 (m, 4H), 2.80 (m, 1H), 2.42 (s, 3H), 1.26 (m, 3H). | 566.30 |
| 18SSS | 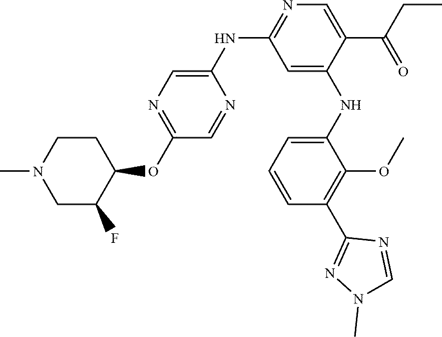 [SMILES: CCC(=O)c1cnc(Nc2cnc(O[C@@H]3CCN(C)C[C@@H]3F)cn2)cc1Nc1cccc(-c2ncn(C)n2)c1OC] | 1H NMR (400 MHz, Methanol-d4) δ 8.83 (m, 1H), 8.53 (m, 2H), 7.92 (m, 1H), 7.66 (m, 2H), 7.32 (m, 2H), 5.15 (m, 1H), 4.04 (s, 3H), 3.73 (s, 3H), 3.10 (m, 3H), 2.9 (m, 1H), 2.7-2.4 (m, 2H), 2.45-2.38 (m, 4H), 2.2 (m, 1H), 2.02 (m, 1H), 1.26 (m, 3H). | 562.20 |
| 18TTT | 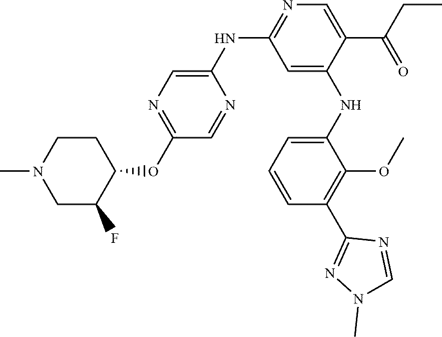 [SMILES: CCC(=O)c1cnc(Nc2cnc(O[C@H]3CCN(C)C[C@@H]3F)cn2)cc1Nc1cccc(-c2ncn(C)n2)c1OC] | 1H NMR (400 MHz, Methanol-d4) δ 8.81 (m, 1H), 8.52 (m, 2H), 7.90 (m, 1H), 7.65 (m, 2H), 7.32 (m, 2H), 5.13 (m, 1H), 4.85-4.65 (m, 1H), 4.04 (s, 3H), 3.73 (s, 3H), 3.13-2.93 (m, 3H), 2.72 (m, 1H), 2.54 (m, 1H), 2.38 (m, 4H), 2.26 (m, 1H), 1.80 (m, 1H), 1.26 (m, 3H). | 562.20 |
| 18UUU | 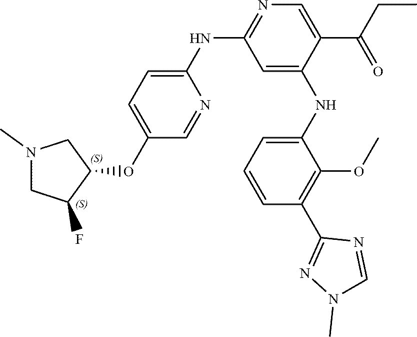 [SMILES: CCC(=O)c1cnc(Nc2ccc(O[C@H]3CN(C)C[C@@H]3F)cn2)cc1Nc1cccc(-c2ncn(C)n2)c1OC] | 1H NMR (400 MHz, Methanol-d4) δ 8.75 (m, 1H), 8.47 (m, 1H), 7.92 (m, 1H), 7.72-7.61 (m, 3H), 7.4-7.27 (m, 3H), 5.18-4.88 (m, 2H), 4.85-4.65 (m, 1H), 4.01 (s, 3H), 3.73 (s, 3H), 3.28 (m, 1H), 3.05 (m, 2H), 2.88 (m, 1H), 2.58 (m, 1H), 2.40 (s, 3H), 1.23 (m, 3H). | 547.40 |
| 18VVV | 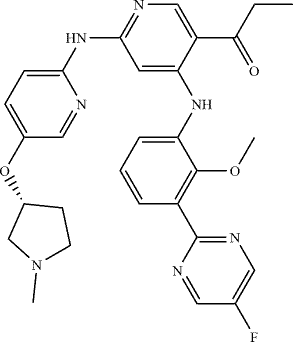 [SMILES: CCC(=O)c1cnc(Nc2ccc(O[C@@H]3CCN(C)C3)cn2)cc1Nc1cccc(-c2ncc(F)cn2)c1OC] | 1H NMR (400 MHz, Methanol-d4) δ 8.89 (m, 2H), 8.77 (m, 1H), 7.87 (m, 1H), 7.74 (m, 1H), 7.58 (m, 2H), 7.40-7.31 (m, 3H), 4.92 (m, 1H), 3.73 (s, 3H), 3.07 (m, 2H), 2.95-2.80 (m, 3H), 2.54-2.35 (m, 5H), 2.00 (m, 1H), 1.28 (m, 3H). | 544.30 |
| 18WWW | 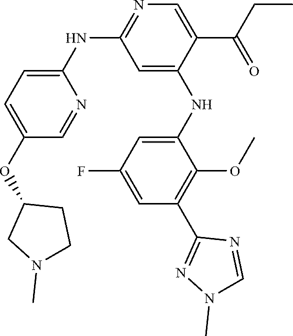 [SMILES: CCC(=O)c1cnc(Nc2ccc(O[C@@H]3CCN(C)C3)cn2)cc1Nc1cc(F)cc(-c2ncn(C)n2)c1OC] | 1H NMR (400 MHz, DMSO-d6) δ 11.30 (s, 1H), 9.92 (s, 1H), 8.86 (m, 1H), 8.61 (m, 1H), 7.95-7.85 (m, 2H), 7.62 (m, 2H), 7.38 (m, 2H), 4.87 (m, 1H), 3.97 (s, 3H), 3.78 (s, 3H), 3.07 (m, 2H), 2.80-2.55 (m, 4H), 2.35-2.25 (m, 4H), 1.78 (m, 1H), 1.15 (m, 3H). | 547.25 |
| 18XXX | 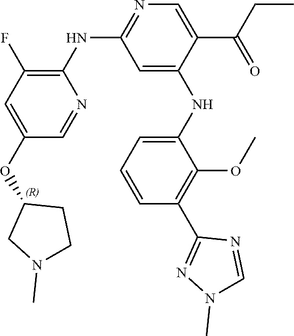 [SMILES: CCC(=O)c1cnc(Nc2ncc(O[C@@H]3CCN(C)C3)cc2F)cc1Nc1cccc(-c2ncn(C)n2)c1OC] | 1H NMR (400 MHz, Methanol-d4) δ 8.76 (m, 1H), 8.50 (m, 1H), 7.88 (m, 1H), 7.76-7.66 (m, 3H), 7.32 (m, 2H), 4.95 (m, 1H), 4.04 (s, 3H), 3.74 (s, 3H), 3.07 (m, 2H), 2.95-2.80 (m, 3H), 2.52 (m, 1H), 2.42-2.36 (m, 4H), 1.98 (m, 1H), 1.25 (m, 3H). | 547.20 |
| 18YYY | 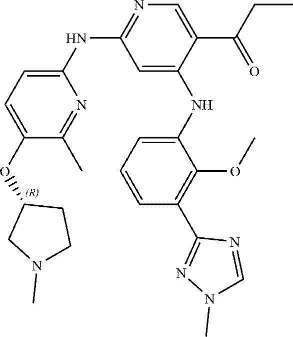 [SMILES: CCC(=O)c1cnc(Nc2ccc(O[C@@H]3CCN(C)C3)c(C)n2)cc1Nc1cccc(-c2ncn(C)n2)c1OC] | 1H NMR (400 MHz, DMSO-d6) δ 11.16 (s, 1H), 9.74 (s, 1H), 8.84 (m, 1H), 8.57 (m, 1H), 8.03 (m, 1H), 7.73-7.62 (m, 2H), 7.31 (m, 3H), 4.79 (m, 1H), 3.97 (s, 3H), 3.74 (s, 3H), 3.08 (m, 2H), 2.80-2.50 (m, 3H), 2.40 (m, 1H), 2.3-2.20 (m, 7H), 1.76 (m, 1H), 1.15 (m, 3H). | 543.20 |
| 18ZZZ | 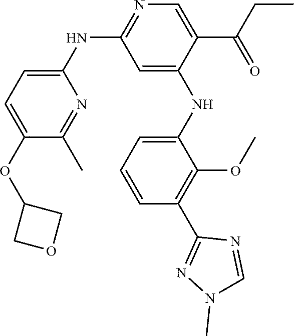 [SMILES: CCC(=O)c1cnc(Nc2ccc(OC3COC3)c(C)n2)cc1Nc1cccc(-c2ncn(C)n2)c1OC] | 1H NMR (400 MHz, Methanol-d4) δ 8.74 (m, 1H), 8.50 (m, 1H), 7.92 (m, 1H), 7.78 (m, 2H), 7.31 (m, 1H), 7.03 (m, 1H), 6.92 (m, 1H), 5.23 (m, 1H), 5.01 (m, 2H), 4.71 (m, 2H), 4.04 (s, 3H), 3.74 (s, 3H), 3.07 (m, 2H), 2.35 (s, 3H), 1.25 (m, 3H). | 516.25 |
| 18AAAA | 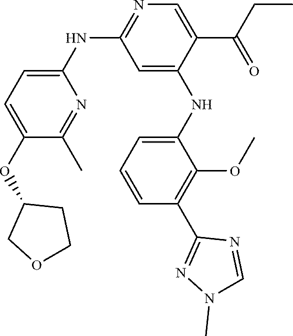 [SMILES: CCC(=O)c1cnc(Nc2ccc(O[C@@H]3CCOC3)c(C)n2)cc1Nc1cccc(-c2ncn(C)n2)c1OC] | 1H NMR (300 MHz, Methanol-d4) δ 8.75 (m, 1H), 8.50 (m, 1H), 7.90 (m, 1H), 7.70 (m, 2H), 7.31 (m, 1H), 7.05 (m, 1H), 4.98 (m, 1H), 4.04-3.90 (m, 7H), 3.74 (s, 3H), 3.08 (m, 2H), 2.31 (s, 3H), 2.25 (m, 1H), 2.12 (m, 1H), 1.26 (m, 3H). | 530.35 |
| 18BBBB | 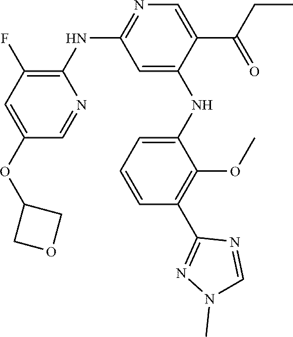 [SMILES: CCC(=O)c1cnc(Nc2ncc(OC3COC3)cc2F)cc1Nc1cccc(-c2ncn(C)n2)c1OC] | 1H NMR (400 MHz, DMSO-d6) δ 11.04 (s, 1H), 9.32 (s, 1H), 8.77 (s, 1H), 8.57 (s, 1H), 7.77 (s, 1H), 7.62 (d, J = 7.1 Hz, 2H), 7.41 - 7.32 (d, J = 12.3 Hz, 3H), 5.40 - 5.33 (m, 1H), 4.94 (t, J = 6.7 Hz, 2H), 4.57 (t, J = 6.0 Hz, 2H), 3.96 (s, 3H), 3.74 (s, 3H), 3.06 (q, J = 7.4 Hz, 2H), 1.12 (t, J = 7.3 Hz, 3H). | 520.2 |
| 18CCCC | 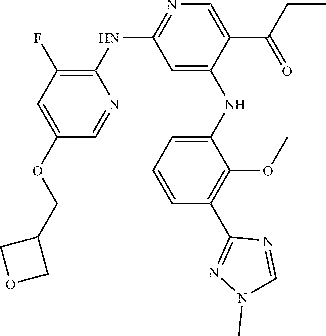 [SMILES: CCC(=O)c1cnc(Nc2ncc(OCC3COC3)cc2F)cc1Nc1cccc(-c2ncn(C)n2)c1OC] | 1H NMR (400 MHz, DMSO-d6) δ 11.05 (s, 1H), 9.28 (s, 1H), 8.77 (s, 1H), 8.57 (s, 1H), 7.92 (s, 1H), 7.70 -7.53 (m, 3H), 7.34 (d, J = 12.3 Hz, 2H), 4.72 (t, J = 7.2 Hz, 2H), 4.55 -4.18 (m, 4H), 3.96 (s, 3H), 3.74 (s, 3H), 3.40 (s, 1H), 3.06 (q, J = 7.8 Hz, 2H), 1.13 (t, J = 7.1 Hz, 3H). | 534.2 |
| 18DDDD | 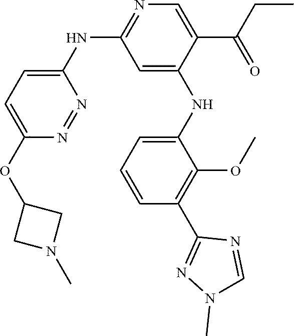 [SMILES: CCC(=O)c1cnc(Nc2ccc(OC3CN(C)C3)nn2)cc1Nc1cccc(-c2ncn(C)n2)c1OC] | 1H NMR (400 MHz, Methanol-d4) δ 8.81 (m, 1H), 8.51 (m, 1H), 8.06 (m, 1H), 7.67 (m, 2H), 7.33 (m, 2H), 7.14 (m, 1H), 5.29 (m, 1H), 4.04 (s, 3H), 3.86 (m, 2H), 3.73 (s, 3H), 3.33 (m, 2H), 3.10 (m, 2H), 2.45 (s, 3H), 1.24 (m, 3H). | 516.15 |

### Example 19

Preparation of 1-(6-((5-(2-amino-1,1-difluoroethyl)pyrazin-2-yl)amino)-4-((2-methoxy-3-(1-methyl-1H-1,2,4-triazol-3-yl)phenyl)amino)pyridin-3-yl)propan-1-one (Compound 19A)

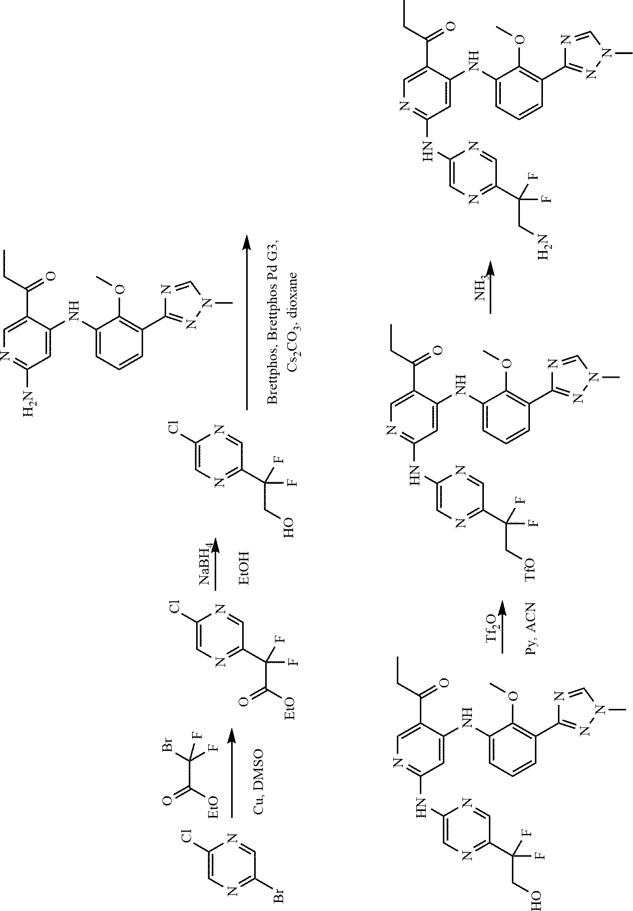

### Step 1: Ethyl 2-(5-chloropyrazin-2-yl)-2,2-difluoroacetate

A solution of 2-bromo-5-chloro-pyrazine (2 g, 10.3 mmol), ethyl 2-bromo-2,2-difluoro-acetate (2.10 g, 10.3 mmol), copper (1.3 g, 20.7 mmol) in dimethylsulfoxide (30 mL) was stirred overnight at room temperature. The resulting mixture was filtered and the filtrate was concentrated under reduced pressure. The residual material was diluted with water (50 ml) and ethyl acetate (100 ml). The biphasic mixture was passed through a celite bed and the filtrate was separated into two layers. The ethyl acetate layer was separated and then washed with water (2 × 30 ml) and saturated aqueous sodium chloride (30 ml), dried over anhydrous sodium sulfate and concentrated and purified by silica gel chromatography eluting with 20% ethyl acetate in petroleum ether to yield ethyl 2-(5-chloropyrazin-2-yl)-2,2-difluoroacetateo-acetate (1.3 g, 53%).

### Step 2: 2-(5-chloropyrazin-2-yl)-2,2-difluoroethan-1-ol

To a solution of ethyl 2-(5-chloropyrazin-2-yl)-2,2-difluoroacetate (500 mg, 2.1 mmol) in ethanol (20 mL) at 0° C., was added sodium borohydride (1.74 g, 4.2 mmol). The mixture was allowed to warm to room temperature and stirred for 1 hour. The mixture was diluted with water (30 mL) and dichloromethane (100 mL) and passed through a celite bed. The filtrate was separated and the dichloromethane layer was washed with water (2 × 30 mL) and saturated aqueous sodium chloride (30 mL). After drying with anhydrous sodium sulfate the reaction mixture was concentrated and purified with silica gel chromatography, eluting with 40% ethyl acetate in petroleum ether to yield 2-(5-chloropyrazin-2-yl)-2,2-difluoroethan-1-ol (200 mg, 49%) as an oil.

### Step 3: 1-(6-((5-(1,1-difluoro-2-hydroxyethyl)pyrazin-2-yl)amino)-4-((2-methoxy-3-(1-methyl-1H-1,2,4-triazol-3-yl)phenyl)amino)pyridin-3-yl)propan-1-one

A solution of 1-(6-amino-4-((2-methoxy-3-(1-methyl-1H-1,2,4-triazol-3-yl)phenyl)amino)pyridin-3-yl)propan-1-one (200 mg, 0.57 mmol), 2-(5-chloropyrazin-2-yl)-2,2-difluoroethan-1-ol (166 mg, 0.85 mmol), Brettphos Pd G3 (51 mg, 0.06 mol), Brettphos (30 mg, 0.06 mmol), cesium carbonate (555 mg, 1.7 mmol) in 1,4-dioxane (10 mL) was stirred at 90° C. for 2 hours under nitrogen. The reaction mixture was concentrated and applied onto a silica gel column and eluted with dichloromethane:methanol (10:1) to yield 1-(6-((5-(1,1-difluoro-2-hydroxyethyl)pyrazin-2-yl)amino)-4-((2-methoxy-3-(1-methyl-1H-1,2,4-triazol-3-yl)phenyl)amino)pyridin-3-yl)propan-1-one (150 mg, 52%) as an oil.

### Step 4: 2,2-difluoro-2-(5-((4-((2-methoxy-3-(1-methyl-1H-1,2,4-triazol-3-yl)phenyl)amino)-5-propionylpyridin-2-yl)amino)pyrazin-2-yl)ethyl trifluoromethanesulfonate

To a solution of 1-(6-((5-(1,1-difluoro-2-hydroxyethyl)pyrazin-2-yl)amino)-4-((2-methoxy-3-(1-methyl-1H-1,2,4-triazol-3-yl)phenyl)amino)pyridin-3-yl)propan-1-one (200 mg, 0.4 mmol), pyridine (46 mg, 0.6 mmol,) in acetonitrile (2 mL) at 0° C. was added trifluoromethanesulfonic anhydride (133 mg, 0.5 mmol) dropwise over 2 minutes under nitrogen. The mixture was allowed to warm to room temperature and stirred for 2 hours. The mixture was concentrated, applied onto a silica gel column and eluted with dichloromethane:methanol (20:1) to yield 2,2-difluoro-2-(5-((4-((2-methoxy-3-(1-methyl-1H-1,2,4-triazol-3-yl)phenyl)amino)-5-propionylpyridin-2-yl)amino)pyrazin-2-yl)ethyl trifluoromethanesulfonate (160 mg, 64%).

### Step 5: 1-(6-((5-(2-amino-1,1-difluoroethyl)pyrazin-2-yl)amino)-4-((2-methoxy-3-(1-methyl-1H-1,2,4-triazol-3-yl)phenyl)amino)pyridin-3-yl)propan-1-one

A solution of 2,2-difluoro-2-(5-((4-((2-methoxy-3-(1-methyl-1H-1,2,4-triazol-3-yl)phenyl)amino)-5-propionylpyridin-2-yl)amino)pyrazin-2-yl)ethyl trifluoromethanesulfonate (60 mg, 0.09 mmol) in 0.4 M ammonia in 1,4-dioxane (4 mL) was stirred overnight at 80° C. The mixture was concentrated and purified by prep HPLC to yield 1-(6-((5-(2-amino-1,1-difluoroethyl)pyrazin-2-yl)amino)-4-((2-methoxy-3-(1-methyl-1H-1,2,4-triazol-3-yl)phenyl)amino)pyridin-3-yl)propan-1-one (13.8 mg, 29%) as a solid. ¹H NMR (400 MHz, Methanol-d₄) δ 8.93-8.86 (m, 2H), 8.52-8.42 (m, 2H), 7.77 (s, 1H), 7.69 (d, J = 7.9 Hz, 2H), 7.34 (t, J = 7.9 Hz, 1H), 4.04 (s, 3H), 3.73 (s, 3H), 3.39 (d, J= 14.3 Hz, 2H), 3.12 (q, J= 7.3 Hz, 2H), 1.26 (t, J= 7.3 Hz, 3H).

$$\text{(ES,} m / z ) : {[ \text{M+H]}}^{+} 510.30.$$

### Preparation of Compounds 19B-19E

Compounds 19B-19E, as indicated in TABLE 14, were prepared in a similar manner and according to the general synthetic schemes and procedures described herein.

TABLE 14

COMPOUNDS 19B THROUGH 19E

| Cmpd. No. | Structure | ¹H NMR | MS (M+H)⁺ |
|---|---|---|---|
| 19B | 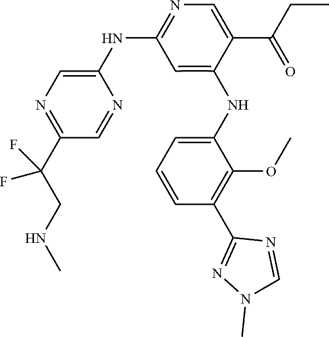 [SMILES: CCC(=O)c1cnc(Nc2cnc(C(F)(F)CNC)cn2)cc1Nc1cccc(-c2ncn(C)n2)c1OC] | 1H NMR (300 MHz, Methanol-d4) δ 8.87 (m, 2H), 8.48 (m, 1H), 8.42 (m, 1H), 7.76 (m, 1H), 7.67 (m, 2H), 7.33 (m, 1H), 4.02 (s, 3H), 3.71 (s, 3H), 3.40-3.30 (m, 2H), 3.07 (m, 2H), 2.42 (m, 3H), 1.28 (m, 3H). | 524.15 |
| 19C | 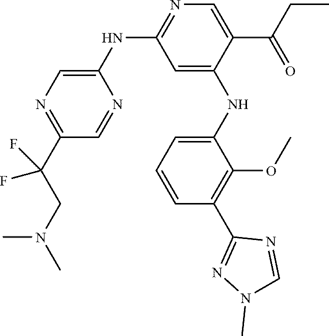 [SMILES: CCC(=O)c1cnc(Nc2cnc(C(F)(F)CN(C)C)cn2)cc1Nc1cccc(-c2ncn(C)n2)c1OC] | 1H NMR (300 MHz, Methanol-d4) δ 8.91 (m, 1H), 8.86 (m, 1H), 8.49 (m, 1H), 8.40 (m, 1H), 7.75 (m, 1H), 7.67 (m, 2H), 7.33 (m, 1H), 4.02 (s, 3H), 3.71 (m, 3H), 3.22-3.06 (m, 4H), 2.32 (s, 6H), 1.24 (m, 3H). | 538.20 |
| 19D | 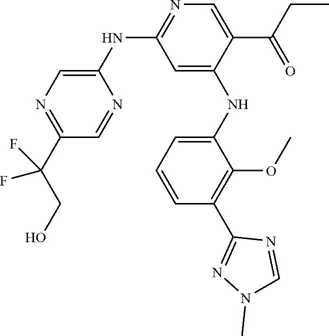 [SMILES: CCC(=O)c1cnc(Nc2cnc(C(F)(F)CO)cn2)cc1Nc1cccc(-c2ncn(C)n2)c1OC] | 1H NMR (300 MHz, DMSO-d6) δ 11.10 (s, 1H), 10.57 (s, 1H), 9.04 (m, 1H), 8.92 (m, 1H), 8.57 (m, 1H), 8.43 (m, 1H), 7.77 (m, 1H), 7.65 (m, 2H), 7.35 (m, 1H), 5.58 (m, 1H), 3.94 (m, 5H), 3.74 (s, 3H), 3.14 (m, 2H), 1.14 (m, 3H). | 511.20 |
| 19E | 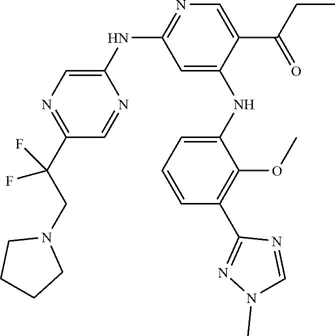 [SMILES: CCC(=O)c1cnc(Nc2cnc(C(F)(F)CN3CCCC3)cn2)cc1Nc1cccc(-c2ncn(C)n2)c1OC] | 1H NMR (300 MHz, Methanol-d4) δ 8.93 (m, 1H), 8.75 (m, 1H), 8.56 (m, 1H), 8.49 (m, 1H), 7.90 (m, 1H), 7.60 (m, 1H), 7.42 (m, 1H), 6.80 (m, 1H), 4.39 (m, 2H), 4.05 (s, 3H), 3.73 (s, 3H), 3.72-3.5 (m, 4H), 3.18 (m, 2H), 2.17 (m, 4H), 1.29 (m, 3H). | 564.35 |

### Example 20

Preparation of 4-((4-((2-methoxy-3-(1-methyl-1H-1,2,4-triazol-3-yl)phenyl)amino)-5-propionylpyridin-2-yl)amino)-1-phenylpyrimidin-2(1H)-one (Compound 20A)

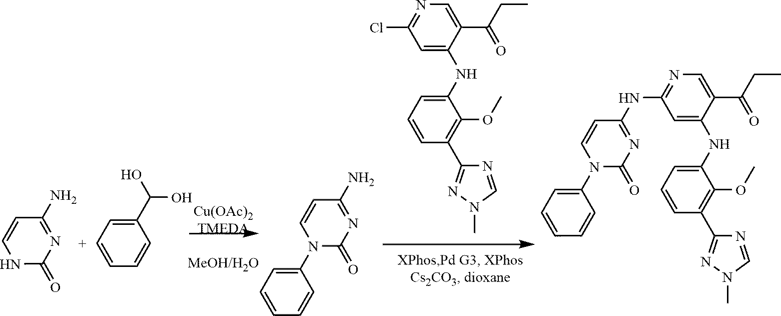

### STEP 1: 4-amino-1-phenylpyrimidin-2(1H)-one

In a 50 ml flask was combined cytosine (200 mg, 1.80 mmol). phenylboronic acid (219 mg, 1.80 mmol), copper(II)acetate (327 mg, 1.80 mmol), N,N,N′,N′-tetramethylethane-1,2-diamine (418 mg, 3.60 mmol), methanol (10 mL) and water (2.5 mL). Stir the mixture vigorously under an atmosphere of air at room temperature for 45 minutes. Concentrated under reduced pressure. The residue was purified by silica gel column chromatography, eluted with dichlorometh ane: methanol (10:1) to yield 4-amino-1-phenyl-pyrimidin-2-one (200 mg, 59%).

### Step 2: 4-((4-((2-methoxy-3-(1-methyl-1H-1,2,4-triazol-3-yl)phenyl)amino)-5-propionylpyridin-2-yl)amino)-1-phenylpyrimidin-2(1H)-one

To a stirred mixture of 1-(6-chloro-4-((2-methoxy-3-(1-methyl-1H-1,2,4-triazol-3-yl)phenyl)amino)pyridin-3-yl)propan-1-one (100 mg, 2.68 mmol) in 1,4-dioxane (10 mL) was added 4-amino-1-phenylpyrimidin-2(1H)-one (76 mg, 4.03 mmol) XPhos (26 mg, 0.54 mmol) Xphos Pd G3 (23 mg, 0.27 mmol) and cesium carbonate (175 mg, 5.4 mol). The mixture was stirred for 12 hours at 90° C. under a nitrogen atmosphere. Concentrated mixture under vacuum and the residue was purified by prep HPLC to yield 4-((4-((2-methoxy-3-(1-methyl-1H-1,2,4-triazol-3-yl)phenyl)amino)-5-propionylpyridin-2-yl)amino)-1-phenylpyrimidin-2(1H)-one (34 mg, 24%) as a solid. ¹H-NMR: (DMSO, 300 MHz, ppm): 11.30 (s, 1H), 10.57 (s, 1H), 8.91 (s, 1H), 8.54 (s, 2H), 7.92 (d, J = 7.3 Hz, 1H), 7.82 (dd, J = 8.3, 1.6 Hz, 1H), 7.60 - 7.35 (m, 6H), 7.24 (t, J = 8.0 Hz, 1H), 6.52 (d, J = 6.8 Hz, 1H), 3.93 (s, 3H), 3.75 (s, 3H), 3.13 (q, J = 7.2 Hz, 2H), 1.12 (t, J = 7.2 Hz, 3H).

$$\text{LC-MS: (ES, m/z): [M+H]+ 523} \text{.15} \text{.}$$

### Example 21

Preparation of 1-(4-((2-methoxy-3-(1-methyl-1H-1,2,4-triazol-3-yl)phenyl)amino)-6-((1-(3-methoxycyclobutyl)-1H-pyrazol-3-yl)amino)pyridin-3-yl)propan-1-one (Compound 21A)

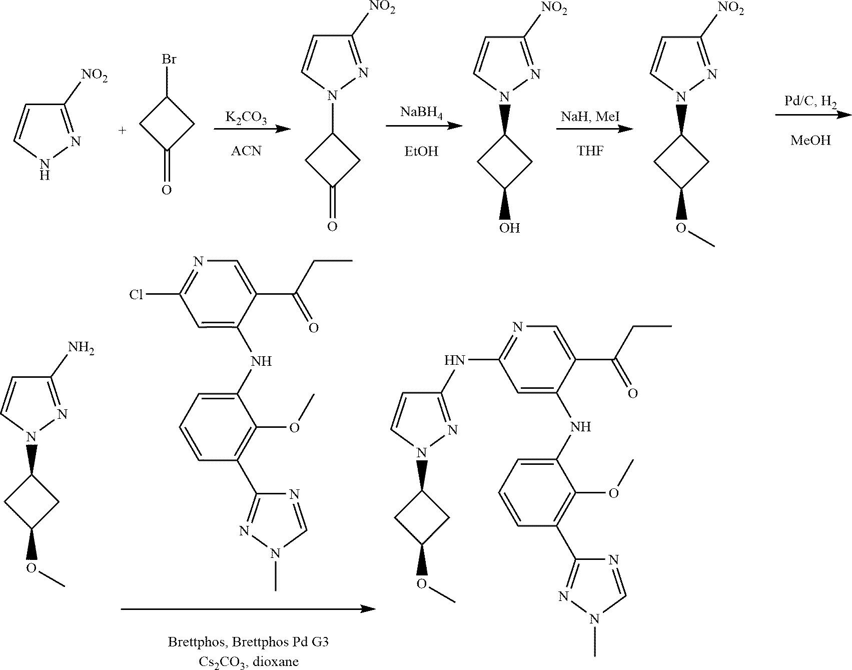

### Step 1: 3-(3-nitro-1H-pyrazol-1-yl)cyclobutan-1-one

Into a round-bottom flask was placed 3-nitro-1H-pyrazole (1 g, 8.8 mmol), acetonitrile (15 mL), potassium carbonate (1.22 g, 8.84 mmol) and 3-bromocyclobutanone (1.32 g, 8.8 mmol) and the mixture was stirred overnight at room temperature. Dichloromethane (100 mL) was added and the solids were filtered off. The filtrate was concentrated under vacuum, then purified on a silica gel column eluting with 0-80% dichloromethane in petroleum ether to yield 3-(3-nitro-1H-pyrazol-1-yl)cyclobutan-1-one (0.85 g, 53%) as a solid.

### Step 2: 3-(3-nitro-1H-pyrazol-1-yl)cyclobutan-1-ol

In a round-bottom flask was combined 3-(3-nitro-1H-pyrazol-1-yl)cyclobutan-1-one (0.85 g, 4.7 mmol) in ethanol (10 mL), followed by the batchwise addition of sodium borohydride (178 mg, 4.7 mmol) at 0° C. The mixture was stirred for 2 hours at 0° C. and then concentrated under vacuum. The residue was purified by silica gel chromatography eluting with a gradient of 0-10% methanol in dichloromethane to yield 3-(3-nitro-1H-pyrazol-1-yl)cyclobutan-1-ol (0.60 g, 70%) as a solid.

### Step 3: 1-(3-methoxycyclobutyl)-3-nitro-1H-pyrazole

To a mixture of 3-(3-nitro-1H-pyrazol-1-yl)cyclobutan-1-ol (200 mg, 1.1 mmol) in tetrahydrofuran (5 mL) under a nitrogen atmosphere was added sodium hydride (66 mg, 1.64 mmol, 60 wt%) at 0° C. and allowed to stirr for 15 minutes at 0° C. To the mixture was added iodomethane (0.27 mL, 4.37 mmol) and stirred at room temperature for 3 hours. The reaction mixture was quenched with methanol (2 mL) and concentrated under vacuum. The residue was purified by Prep TLC (DCM:MeOH; 10:1) to yield 1-(3-methoxycyclobutyl)-3-nitro-1H-pyrazole (110 mg, 51%) as a solid.

### Step 4: 1-(3-methoxycyclobutyl)-1H-pyrazol-3-amine

Into a round-bottom flask was added 1-(3-methoxycyclobutyl)-3-nitro-1H-pyrazole (110 mg, 0.55 mmol) and palladium on carbon (100 mg, 10 wt%) in methanol (5 mL). The atmosphere was purged and refilled with hydrogen gas. Stirred at room temperature for 3 hours. Purged atmosphere with nitrogen, filtered off solids over celite, washing with methanol (50 mL). The filtrate was concentrated under reduced pressure to yield crude 1-(3-methoxycyclobutyl)-1H-pyrazol-3-amine (80 mg, 86%) which was used in the next step without further purification.

### Step 5. 1-(4-((2-methoxy-3-(1-methyl-1H-1,2,4-triazol-3-yl)phenyl)amino)-6-((1-(3-methoxycyclobutyl)-1H-pyrazol-3-yl)amino)pyridin-3-yl)propan-1-one

To 1-(6-chloro-4-((2-methoxy-3-(1-methyl-1H-1,2,4-triazol-3-yl)phenyl)amino)pyridin-3-yl)propan-1-one (150 mg, 0.4 mmol) in 1,4-dioxane (6 mL) was added 1-(3-methoxycyclobutyl)-1H-pyrazol-3-amine (81 mg, 0.48 mmol), Brettphos Pd G3 (37 mg, 0.04 mmol), Brettphos (74 mg, 0.08 mmol), and cesium carbonate (677 mg, 0.8 mmol). The mixture was stirred for 4 hours at 100° C. under a nitrogen atmosphere. After filtrating and concentrating under vacuum, the residue was purified by prep HPLC to yield 1-(4-((2-methoxy-3-(1-methyl-1H-1,2,4-triazol-3-yl)phenyl)amino)-6-((1-(3-methoxycyclobutyl)-1H-pyrazol-3-yl)amino)pyridin-3-yl)propan-1-one (110 mg, 54%) as a solid. ¹H NMR (400 MHz, Methanol-d4) δ 8.80 (s, 1H), 8.50 (s, 1H), 7.75 - 7.65 (m, 2H), 7.58 (s, 1H), 7.36 (t, J = 7.9 Hz, 1H), 7.11 (s, 1H), 6.06 (d, J = 2.4 Hz, 1H), 4.42 -4.30 (m, 1H), 4.04 (s, 3H), 3.82 - 3.72 (m, 1H), 3.71 (s, 3H), 3.29 (s, 3H), 3.07 (q, J = 7.3 Hz, 2H), 2.85 - 2.73 (m, 2H), 2.45 - 2.35 (m, 2H), 1.26 (t, J = 7.3 Hz, 3H).

$$\text{(ES, m/z): [M+H]+ 503} \text{.15} \text{.}$$

### Preparation of Compounds 21B-21GG

Compounds 21B-21GG, as indicated in TABLE 15, were prepared in a similar manner and according to the general synthetic schemes and procedures described herein.

TABLE 15

COMPOUNDS 21B THROUGH 21GG

| Cmpd. No. | Structure | ¹H NMR | MS (M+H)⁺ |
|---|---|---|---|
| 21B | 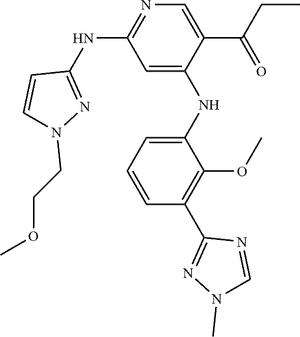 [SMILES: CCC(=O)c1cnc(Nc2ccn(CCOC)n2)cc1Nc1cccc(-c2ncn(C)n2)c1OC] | ¹H NMR (400 MHz, Methanol-d4) δ 8.88 (m, 1H), 8.52 (m, 1H), 7.86 (m, 1H), 7.64 (m, 1H), 7.58 (m, 1H), 7.37 (m, 1H), 6.46 (m, 1H), 5.94 (m, 1H), 4.31 (m, 2H), 4.03 (s, 3H), 3.80-3.72 (m, 5H), 3.3 (s, 3H), 3.13 (m, 2H), 1.26 (m, 3H). | 477.15 |
| 21C | 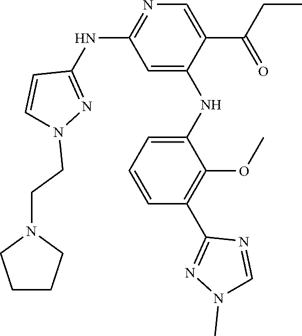 [SMILES: CCC(=O)c1cnc(Nc2ccn(CCN3CCCC3)n2)cc1Nc1cccc(-c2ncn(C)n2)c1OC] | 1H NMR (300 MHz, DMSO-d6) δ 11.12 (s, 1H), 9.62 (s, 1H), 8.78 (m, 1H), 8.60 (m, 1H), 7.68-7.58 (m, 4H), 7.28 (m, 1H), 6.07 (m, 1H), 4.10 (m, 2H), 3.96 (s, 3H), 3.75 (s, 3H), 3.05 (m, 2H), 2.82 (m, 2H), 2.55-2.46 (m, 4H), 1.64 (m, 4H), 1.14 (m, 3H). | 516.30 |
| 21D | 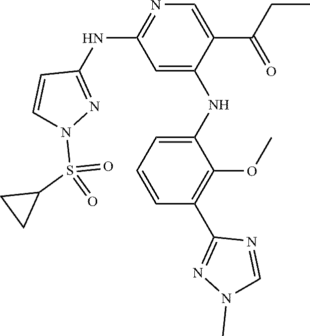 [SMILES: CCC(=O)c1cnc(Nc2ccn(S(=O)(=O)C3CC3)n2)cc1Nc1cccc(-c2ncn(C)n2)c1OC] | 1H NMR (400 MHz, Methanol-d4) δ 8.92 (m, 1H), 8.81 (m, 1H), 8.15 (m, 1H), 7.91 (m, 1H), 7.68 (m, 1H), 7.44 (m, 1H), 7.18 (m, 1H), 6.34 (m, 1H), 4.08 (s, 3H), 3.75 (s, 3H), 3.14 (m, 2H), 2.89 (m, 1H), 1.29 (m, 5H), 1.13 (m, 2H). | 523.10 |
| 21E | 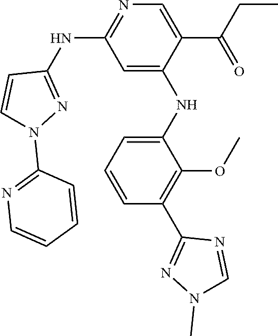 [SMILES: CCC(=O)c1cnc(Nc2ccn(-c3ccccn3)n2)cc1Nc1cccc(-c2ncn(C)n2)c1OC] | 1H NMR (300 MHz, DMSO-d6) δ 11.02 (s, 1H), 9.98 (s, 1H), 8.81 (m, 1H), 8.55 (m, 1H), 8.47 (m, 1H), 8.35 (m, 1H), 7.88-7.66 (m, 4H), 7.34-7.21 (m, 3H), 6.36 (m, 1H), 3.93 (s, 3H), 3.72 (s, 3H), 3.08 (m, 2H), 1.14 (m, 3H). | 496.10 |
| 21F | 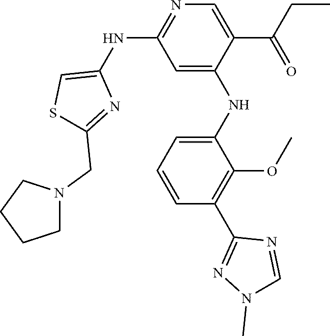 [SMILES: CCC(=O)c1cnc(Nc2csc(CN3CCCC3)n2)cc1Nc1cccc(-c2ncn(C)n2)c1OC] | 1H NMR (300 MHz, Methanol-d4) δ 8.79 (m, 1H), 8.47 (m, 1H), 7.58 (m, 2H), 7.26 (m, 2H), 6.82 (s, 1H), 4.01 (s, 3H), 3.89 (s, 2H), 3.70 (s, 3H), 3.04 (m, 2H), 2.66 (m, 4H), 1.82 (m, 4H), 1.24 (m, 3H). | 519.35 |
| 21G | 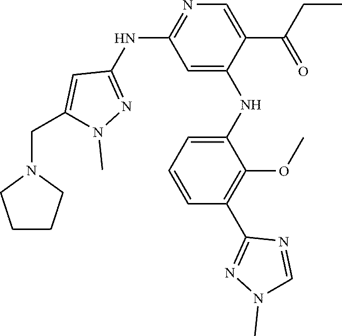 [SMILES: CCC(=O)c1cnc(Nc2cc(CN3CCCC3)n(C)n2)cc1Nc1cccc(-c2ncn(C)n2)c1OC] | 1H NMR (400 MHz, Methanol-d4) δ 8.71 (m, 1H), 8.47 (m, 1H), 7.65 (m, 2H), 7.26 (m, 2H), 6.12 (s, 1H), 4.01 (s, 3H), 3.75-3.71 (m, 8H), 3.4 (m, 2H), 2.68 (m, 4H), 1.85 (m, 4H), 1.23 (m, 3H). | 516.15 |
| 21H | 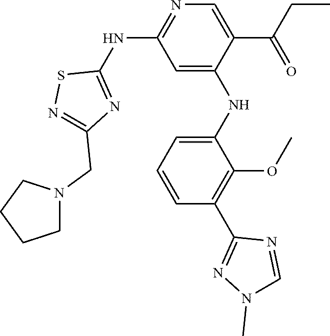 [SMILES: CCC(=O)c1cnc(Nc2nc(CN3CCCC3)ns2)cc1Nc1cccc(-c2ncn(C)n2)c1OC] | 1H NMR (400 MHz, Methanol-d4) δ 9.00 (m, 1H), 8.49 (m, 1H), 7.69 (m, 1H), 7.57 (m, 1H), 7.30 (m, 1H), 6.71 (m, 1H), 4.02 (s, 3H), 3.84 (m, 2H), 3.69 (s, 3H), 3.13 (m, 2H), 2.72 (m, 4H), 1.82 (m, 4H), 1.26 (m, 3H). | 520.20 |
| 21I | 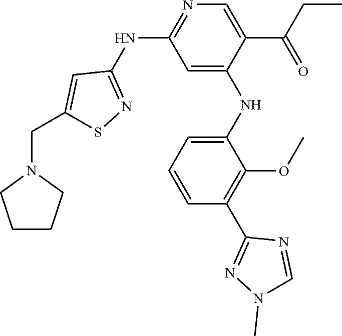 [SMILES: CCC(=O)c1cnc(Nc2cc(CN3CCCC3)sn2)cc1Nc1cccc(-c2ncn(C)n2)c1OC] | 1H NMR (400 MHz, Methanol-d4) δ 8.78 (m, 1H), 8.48 (m, 1H), 7.91 (m, 1H), 7.67 (m, 2H), 7.29 (m, 1H), 7.07 (m, 1H), 4.02 (s, 3H), 3.93 (m, 2H), 3.72 (s, 3H), 3.06 (m, 2H), 2.65 (m, 4H), 1.83 (m, 4H), 1.24 (m, 3H). | 519.20 |
| 21J | 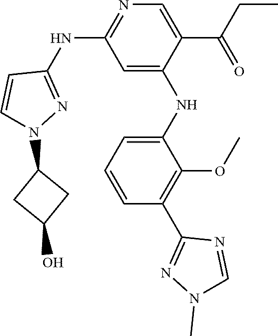 [SMILES: CCC(=O)c1cnc(Nc2ccn([C@H]3C[C@@H](O)C3)n2)cc1Nc1cccc(-c2ncn(C)n2)c1OC] | 1H NMR (400 MHz, Methanol-d4) δ 8.73 (s, 1H), 8.50 (s, 1H), 7.70 - 7.60 (m, 2H), 7.54 (d, J = 2.4 Hz, 1H), 7.34 (t, J = 7.9 Hz, 1H), 7.25 (s, 1H), 6.12 (d, J = 2.3 Hz, 1H), 4.31 - 4.18 (m, 1H), 4.15 - 4.07 (m, 1H), 4.04 (s, 3H), 3.74 (s, 3H), 3.06 (q, J = 7.3 Hz, 2H), 2.85 - 2.74 (m, 2H), 2.40 - 2.30 (m, 2H), 1.25 (t, J = 7.4 Hz, 3H). | 489.2 |
| 21K | 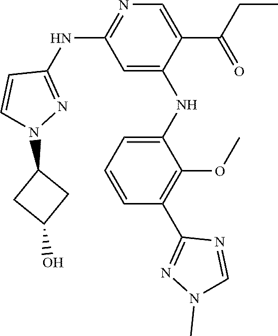 [SMILES: CCC(=O)c1cnc(Nc2ccn([C@H]3C[C@H](O)C3)n2)cc1Nc1cccc(-c2ncn(C)n2)c1OC] | 1H NMR (300 MHz, Methanol-d4) δ 8.70 (s, 1H), 8.47 (s, 1H), 7.64 (d, J = 7.9 Hz, 2H), 7.51 - 7.40 (m, 2H), 7.27 (t, J = 7.9 Hz, 1H), 6.01 (d, J = 2.4 Hz, 1H), 4.85 -4.72 (m, 1H), 4.38 (t, J = 3.7 Hz, 1H), 4.01 (s, 3H), 3.72 (s, 3H), 3.03 (q, J = 7.4 Hz, 2H), 2.76 - 2.51 (m, 2H), 2.50 - 2.12 (m, 2H), 1.23 (t, J = 7.3 Hz, 3H). | 503.2 |
| 21L | 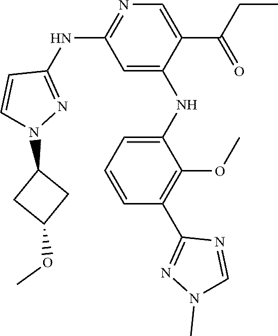 [SMILES: CCC(=O)c1cnc(Nc2ccn([C@H]3C[C@H](OC)C3)n2)cc1Nc1cccc(-c2ncn(C)n2)c1OC] | 1H NMR (300 MHz, DMSO-d6) δ 11.09 (s, 1H), 10.38 (s, 1H), 8.89 (s, 1H), 8.58 (s, 1H), 7.81 - 7.72 (m, 2H), 7.63 - 7.54 (m, 1H), 7.32 (t, J = 7.9 Hz, 1H), 7.10 (s, 1H), 6.05 (d, J = 2.3 Hz, 1H), 4.86 (t, J = 7.6 Hz, 1H), 3.96 (s, 4H), 3.78 (s, 3H), 3.13 (m, 5H), 2.38 (s, 2H), 2.35 - 2.26 (m, 2H) 1.15 (t, J = 7.2 Hz, 3H). | 503.2 |
| 21M | 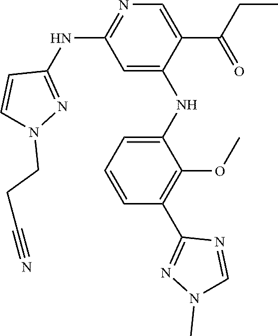 [SMILES: CCC(=O)c1cnc(Nc2ccn(CCC#N)n2)cc1Nc1cccc(-c2ncn(C)n2)c1OC] | 1H NMR (300 MHz, Methanol-d4) δ 8.73 (s, 1H), 8.50 (s, 1H), 7.69 (d, J = 7.9 Hz, 2H), 7.58 - 7.49 (m, 2H), 7.33 (t, J = 7.9 Hz, 1H), 6.08 (d, J = 2.4 Hz, 1H), 4.26 (t, J = 6.5 Hz, 2H), 4.04 (s, 3H), 3.73 (s, 3H), 3.06 (q, J = 7.3 Hz, 2H), 2.87 (t, J = 6.5 Hz, 2H), 1.26 (t, J = 7.3 Hz, 3H). | 472.2 |
| 21N | 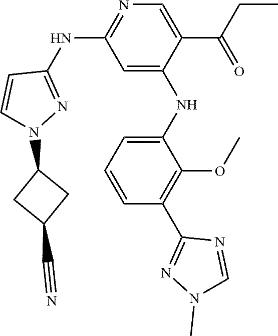 [SMILES: CCC(=O)c1cnc(Nc2ccn([C@H]3C[C@@H](C#N)C3)n2)cc1Nc1cccc(-c2ncn(C)n2)c1OC] | 1H NMR (300 MHz, Methanol-d4) δ 8.78 (s, 1H), 8.49 (s, 1H), 7.73 (dd, J = 7.9, 1.6 Hz, 1H), 7.67 - 7.54 (m, 2H), 7.35 (t, J = 7.9 Hz, 1H), 7.07 (s, 1H), 6.09 (d, J = 2.4 Hz, 1H), 4.81 - 4.64 (m, 1H), 4.03 (s, 3H), 3.73 (s, 3H), 3.18 - 3.07 (m, 2H), 3.04 (d, J = 7.3 Hz, 1H), 2.92 - 2.76 (m, 4H), 1.25 (t, J = 7.3 Hz, 3H). | 498.2 |
| 21O | 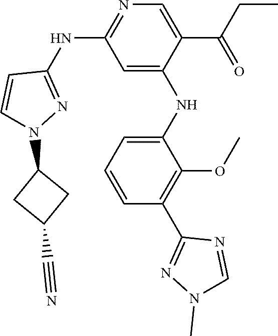 [SMILES: CCC(=O)c1cnc(Nc2ccn([C@H]3C[C@H](C#N)C3)n2)cc1Nc1cccc(-c2ncn(C)n2)c1OC] | 1H NMR (400 MHz, Methanol-d4) δ 8.73 (s, 1H), 8.49 (s, 1H), 7.73 (dd, J = 7.8, 1.6 Hz, 1H), 7.64 (dd, J = 7.9, 1.6 Hz, 1H), 7.53 (s, 1H), 7.47 (d, J = 2.4 Hz, 1H), 7.31 (t, J = 7.9 Hz, 1H), 6.00 (d, J = 2.4 Hz, 1H), 4.97 (dd, J = 8.5, 7.4 Hz, 1H), 4.04 (s, 3H), 3.75 (s, 3H), 3.16 - 3.01 (m, 3H), 2.82 - 2.61 (m, 4H), 1.26 (t, J = 7.3 Hz, 3H). | 498.2 |
| 21P | 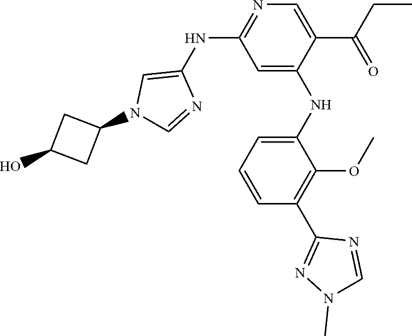 [SMILES: CCC(=O)c1cnc(Nc2cn([C@H]3C[C@@H](O)C3)cn2)cc1Nc1cccc(-c2ncn(C)n2)c1OC] | 1H NMR (400 MHz, DMSO-d6) δ 10.98 (s, 1H), 9.52 (s, 1H), 8.84 (s, 1H), 8.57 (s, 1H), 7.59 (d, J = 7.9 Hz, 2H), 7.51 (s, 1H), 7.35 (s, 1H), 7.25 (t, J = 7.9 Hz, 1H), 6.81 (s, 1H), 5.32 (d, J = 6.8 Hz, 1H), 4.25 - 4.18 (m, 1H), 3.96 (s, 4H), 3.74 (s, 3H), 3.10 - 3.03 (m, 2H), 2.83 - 2.75 (m, 2H), 2.21 - 2.13 (m, 2H), 1.13 (t, J = 7.3 Hz, 3H). | 489.2 |
| 21Q | 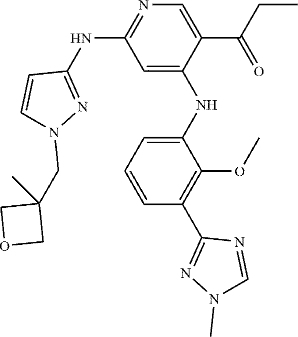 [SMILES: CCC(=O)c1cnc(Nc2ccn(CC3(C)COC3)n2)cc1Nc1cccc(-c2ncn(C)n2)c1OC] | 1H NMR (300 MHz, DMSO-d6) δ 11.06 (s, 1H), 9.63 (s, 1H), 8.78 (s, 1H), 8.57 (s, 1H), 7.66 - 7.57 (m, 3H), 7.42 (s, 1H), 7.28 (t, J = 7.9 Hz, 1H), 6.15 (s, 1H), 4.49 (d, J = 5.9 Hz, 2H), 4.18 - 4.08 (m, 4H), 3.96 (s, 3H), 3.73 (s, 3H), 3.05 (q, J = 7.3 Hz, 2H), 1.18 - 1.05 (m, 6H). | 503.3 |
| 21R | 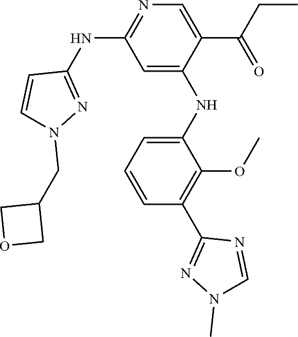 [SMILES: CCC(=O)c1cnc(Nc2ccn(CC3COC3)n2)cc1Nc1cccc(-c2ncn(C)n2)c1OC] | 1H NMR (400 MHz, Methanol-d4) δ 8.72 (s, 1H), 8.50 (s, 1H), 7.72 - 7.69 (m, 2H), 7.50 (d, J = 2.3 Hz, 1H), 7.46 (s, 1H), 7.34 (t, J = 7.9 Hz, 1H), 6.05 (d, J = 2.3 Hz, 1H), 4.75 - 4.65 (m, 2H), 4.44 (t, J = 6.1 Hz, 2H), 4.29 (d, J = 7.3 Hz, 2H), 4.04 (s, 3H), 3.74 (s, 3H), 3.38 (d, J = 7.0 Hz, 1H), 3.06 (q, J = 7.3 Hz, 2H), 1.25 (t, J = 7.3 Hz, 3H). | 489.2 |
| 21S | 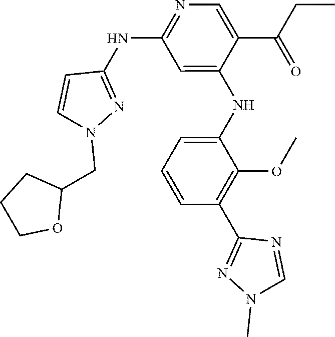 [SMILES: CCC(=O)c1cnc(Nc2ccn(CC3CCCO3)n2)cc1Nc1cccc(-c2ncn(C)n2)c1OC] | 1H NMR (400 MHz, Methanol-d4) δ 8.91 (s, 1H), 8.62 (s, 1H), 7.93 - 7.82 (m, 1H), 7.66 (s, 1H), 7.65 - 7.58 (m, 1H), 7.38 (t, J = 7.9 Hz, 1H), 6.63 - 6.58 (m, 1H), 5.99 (s, 1H), 4.35 - 4.21 (m, 2H), 4.20 - 4.13 (m, 1H), 4.06 (s, 3H), 3.90 - 3.82 (m, 1H), 3.82 - 3.73 (m, 1H), 3.74 (s, 3H), 3.13 (q, J = 7.2 Hz, 2H), 2.12 - 1.99 (m, 1H), 1.97-1.75 (m, 2H), 1.74 - 1.61 (m, 1H), 1.27 (t, J = 7.2 Hz, 3H). | 503.2 |
| 21T | 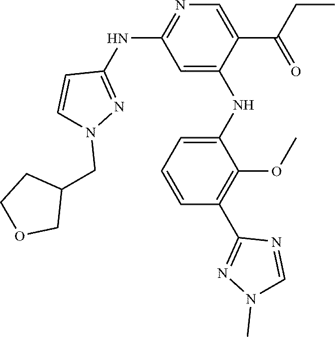 [SMILES: CCC(=O)c1cnc(Nc2ccn(CC3CCOC3)n2)cc1Nc1cccc(-c2ncn(C)n2)c1OC] | 1H NMR (300 MHz, Methanol-d4) δ 8.73 (s, 1H), 8.49 (s, 1H), 7.68 (d, J = 7.9 Hz, 2H), 7.53 - 7.45 (m, 2H), 7.35 - 7.24 (m, 1H), 6.04 (d, J = 2.3 Hz, 1H), 4.03 (s, 3H), 3.97 (d, J = 7.6 Hz, 2H), 3.85 - 3.78 (m, 1H), 3.74 (s, 3H), 3.76 - 3.61 (m, 2H), 3.48 (dd, J = 8.8, 5.4 Hz, 1H), 3.05 (q, J = 7.4 Hz, 2H), 2.75 - 2.65 (m, 1H), 1.97 - 1.90 (m, 1H), 1.55 - 1.45 (m, 1H), 1.25 (t, J = 7.3 Hz, 3H). | 503.2 |
| 21U | 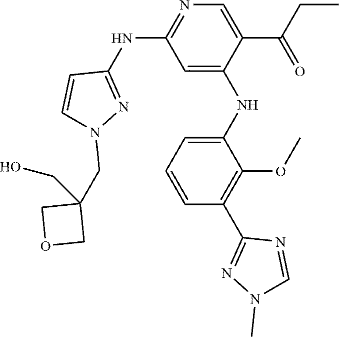 [SMILES: CCC(=O)c1cnc(Nc2ccn(CC3(CO)COC3)n2)cc1Nc1cccc(-c2ncn(C)n2)c1OC] | 1H NMR (400 MHz, Methanol-d4) δ 8.72 (s, 1H), 8.49 (s, 1H), 7.85 - 7.62 (m, 2H), 7.49 (d, J = 2.4 Hz, 1H), 7.42 (s, 1H), 7.34 (t, J = 7.9 Hz, 1H), 6.09 (d, J = 2.3 Hz, 1H), 4.55 (d, J = 6.3 Hz, 2H), 4.33 -4.27 (m, 4H), 4.03 (s, 3H), 3.73 (s, 3H), 3.55 (s, 2H), 3.05 (q, J = 7.4 Hz, 2H), 1.24 (t, J = 7.3 Hz, 3H). | 519.2 |
| 21V | 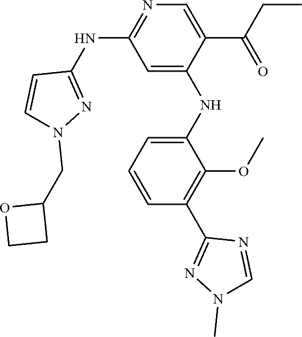 [SMILES: CCC(=O)c1cnc(Nc2ccn(CC3CCO3)n2)cc1Nc1cccc(-c2ncn(C)n2)c1OC] | ¹H NMR (400 MHz, DMSO-d6) δ 8.89 (s, 1H), 8.55 (s, 1H), 7.90 (s, 1H), 7.70 (s, 1H), 7.59 (d, J = 7.6 Hz, 1H), 7.42 - 7.31 (m, 1H), 6.58 - 6.42 (dd, J = 7.9, 1.7 Hz, 1H), 6.07 - 5.90 (t, J = 7.9 Hz, 1H), 5.20 (d, J = 12.3 Hz, 1H), 4.72 - 4.31 (m, 3H), 4.30 - 4.05 (m, 1H), 4.36 - 4.18 (m, 3H), 3.96 (s, 3H), 3.11 (q, J = 7.2 Hz, 2H), 2.83 - 2.70 (m, 1H), 2.58 - 2.39 (m, 1H), 1.25 (t, J = 7.2 Hz, 3H). | 489.2 |
| 21W | 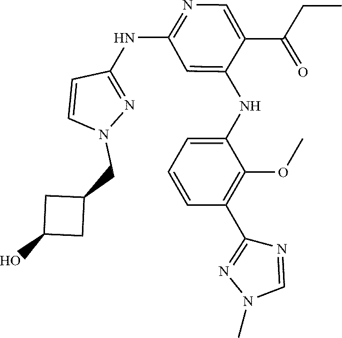 [SMILES: CCC(=O)c1cnc(Nc2ccn(C[C@H]3C[C@@H](O)C3)n2)cc1Nc1cccc(-c2ncn(C)n2)c1OC] | 1H NMR (300 MHz, Methanol-d4) δ 8.61 (s, 1H), 8.37 (s, 1H), 7.81 - 7.48 (m, 3H), 7.46 (d, J = 2.4 Hz, 1H), 7.18 (t, J = 7.9 Hz, 1H), 7.06 (s, 1H), 5.97 (d, J = 2.4 Hz, 1H), 4.52 - 4.38 (m, 1H), 3.91 (s, 3H), 3.61 (s, 3H), 2.93 (q, J = 7.3 Hz, 2H), 2.46-2.29 (m, 1H), 2.20 - 1.79 (m, 5H), 1.13 (t, J = 7.3 Hz, 3H). | 503.2 |
| 21X | 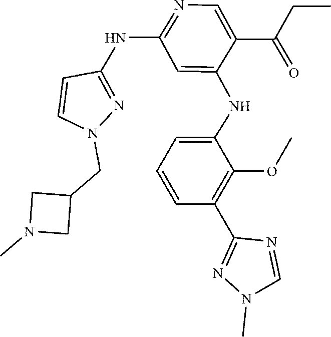 [SMILES: CCC(=O)c1cnc(Nc2ccn(CC3CN(C)C3)n2)cc1Nc1cccc(-c2ncn(C)n2)c1OC] | 1H NMR (400 MHz, Methanol-d4) δ 8.95 (d, J = 17.7 Hz, 1H), 8.56 (s, 1H), 7.88 (dd, J = 7.9, 1.7 Hz, 1H), 7.71 (d, J = 2.4 Hz, 1H), 7.59 (dd, J = 8.0, 1.6 Hz, 1H), 7.40 (t, J = 7.9 Hz, 1H), 6.60 (s, 1H), 6.04 (d, J = 2.7 Hz, 1H), 4.54 - 4.26 (m, 4H), 4.05 (s, 5H), 3.74 (s, 3H), 3.54 - 3.35 (m, 1H), 3.14 (m, 2H), 2.91 (m, 3H), 1.28 (t, J = 7.2 Hz, 3H). | 502.3 |
| 21Y | 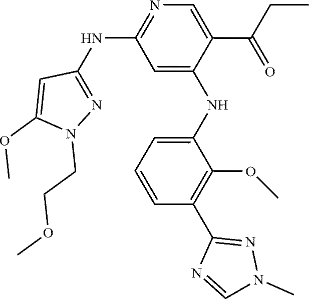 [SMILES: CCC(=O)c1cnc(Nc2cc(OC)n(CCOC)n2)cc1Nc1cccc(-c2ncn(C)n2)c1OC] | 1H NMR (300 MHz, DMSO-d6) δ 11.12 (s, 1H), 9.59 (s, 1H), 8.78 (s, 1H), 8.57 (s, 1H), 7.64 (dd, J = 15.5, 7.9 Hz, 3H), 7.26 (t, J = 7.9 Hz, 1H), 5.55 (s, 1H), 3.96 (s, 3H), 3.90 - 3.80 (m, 5H), 3.75 (s, 3H), 3.55 (t, J = 5.5 Hz, 2H), 3.16 (s, 3H), 3.06 (q, J = 7.2 Hz, 2H), 1.12 (t, J = 7.2 Hz, 3H). | 507.3 |
| 21Z | 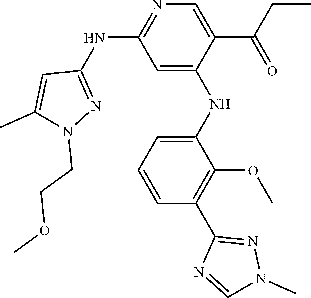 [SMILES: CCC(=O)c1cnc(Nc2cc(C)n(CCOC)n2)cc1Nc1cccc(-c2ncn(C)n2)c1OC] | ¹H NMR (300 MHz, DMSO-d4) δ 11.11 (s, 1H), 9.54 (s, 1H), 8.75 (s, 1H), 8.56 (s, 1H), 7.78 - 7.48 (m, 3H), 7.35 - 7.12 (m, 1H), 5.87 (s, 1H), 4.09 - 3.82 (m, 5H), 3.82 - 3.68 (m, 3H), 3.62 - 3.48 (m, 2H), 3.16 (s, 3H), 3.11 - 2.95 (m, 2H), 2.19 (s, 3H), 1.21 - 1.01 (m, 3H). | 491.0 |
| 21AA | 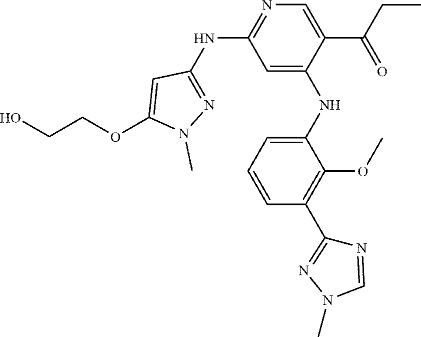 [SMILES: CCC(=O)c1cnc(Nc2cc(OCCO)n(C)n2)cc1Nc1cccc(-c2ncn(C)n2)c1OC] | 1H NMR (400 MHz, DMSO-d6) δ 11.13 (s, 1H), 9.53 (s, 1H), 8.77 (s, 1H), 8.57 (s, 1H), 7.70 - 7.59 (m, 3H), 7.29 (t, J = 7.9 Hz, 1H), 5.56 (s, 1H), 4.95 (s, 1H), 4.05 (t, J = 4.8 Hz, 2H), 3.96 (s, 3H), 3.80 -3.70 (m, 5H), 3.45 - 3.34 (m, 3H), 3.05 (q, J = 7.2 Hz, 2H), 1.13 (t, J = 7.2 Hz, 3H). | 493.3 |
| 21BB | 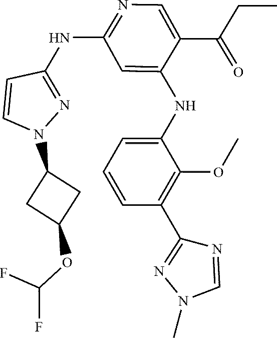 [SMILES: CCC(=O)c1cnc(Nc2ccn([C@H]3C[C@@H](OC(F)F)C3)n2)cc1Nc1cccc(-c2ncn(C)n2)c1OC] | 1H NMR (400 MHz, Methanol-d4) δ 8.73 (s, 1H), 8.49 (s, 1H), 7.74 - 7.55 (m, 2H), 7.52 (d, J = 2.4 Hz, 1H), 7.39 - 7.21 (m, 2H), 6.37 (t, J = 74.9 Hz, 1H), 6.09 (d, J = 2.4 Hz, 1H), 4.60 - 4.20 (m, 2H), 4.03 (s, 3H), 3.74 (s, 3H), 3.05 (q, J = 7.3 Hz, 2H), 2.83 - 2-76 (m, 2H), 2.73 - 2.46 (m, 2H), 1.25 (t, J = 7.3 Hz, 3H). | 539.1 |
| 21CC | 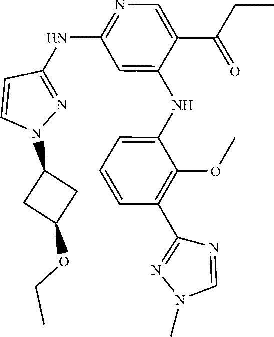 [SMILES: CCO[C@H]1C[C@@H](n2ccc(Nc3cc(Nc4cccc(-c5ncn(C)n5)c4OC)c(C(=O)CC)cn3)n2)C1] | 1H NMR (400 MHz, Methanol-d4) δ 8.73 (s, 1H), 8.49 (s, 1H), 7.76 - 7.62 (m, 2H), 7.53 (d, J = 2.4 Hz, 1H), 7.40 - 7.22 (m, 2H), 6.10 (d, J = 2.4 Hz, 1H), 4.32 - 4.27 (m, 1H), 4.03 (s, 3H), 3.90 - 3.78 (m, 1H), 3.75 (s, 3H), 3.46 (q, J = 7.0 Hz, 2H), 3.05 (d, J = 7.4 Hz, 2H), 2.82 - 2.66 (m, 2H), 2.46 - 2.28 (m, 2H), 1.25 (t, J = 7.3 Hz, 3H), 1.17 (t, J = 7.0 Hz, 3H). | 517.1 |
| 21DD | 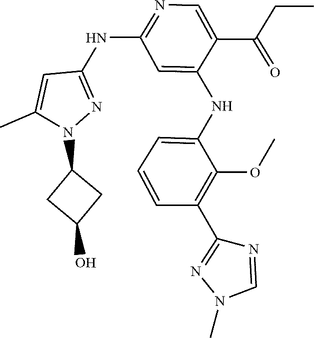 [SMILES: CCC(=O)c1cnc(Nc2cc(C)n([C@H]3C[C@@H](O)C3)n2)cc1Nc1cccc(-c2ncn(C)n2)c1OC] | 1H NMR (400 MHz, Methanol-d4) δ 9.07 (s, 1H), 8.56 (s, 1H), 7.86 (d, J = 8.0 Hz, 1H), 7.59 (d, J = 7.9 Hz, 1H), 7.40 (t, J = 7.9 Hz, 1H), 6.40 (d, J = 5.4 Hz, 1H), 5.77 (s, 1H), 4.47 - 4.43 (m, 1H), 4.25 -4.22 (m, 1H), 4.05 (s, 3H), 3.74 (s, 3H), 3.20 - 3.12 (m, 2H), 2.87 (s, 2H), 2.68 -2.61 (m, 2H), 2.29 (s, 3H), 1.29 (t, J = 7.1 Hz, 3H). | 503.1 |
| 21EE | 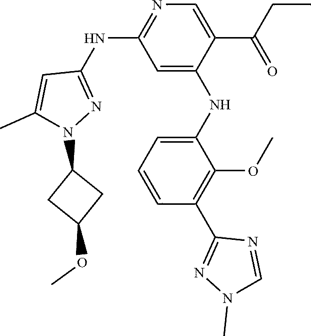 [SMILES: CCC(=O)c1cnc(Nc2cc(C)n([C@H]3C[C@@H](OC)C3)n2)cc1Nc1cccc(-c2ncn(C)n2)c1OC] | 1H NMR (300 MHz, Methanol-d4) δ 9.05 (s, 1H), 8.54 (s, 1H), 7.86 (dd, J = 7.8, 1.7 Hz, 1H), 7.59 (dd, J = 7.9, 1.6 Hz, 1H), 7.39 (t, J = 7.9 Hz, 1H), 6.42 (s, 1H), 5.79 (s, 1H), 4.61 - 4.40 (m, 1H), 4.05 (s, 3H), 3.99 - 3.80 (m, 1H), 3.74 (s, 3H), 3.39 (s, 3H), 3.16 (q, J = 7.2 Hz, 2H), 2.94 - 2.79 (m, 2H), 2.61 (q, J = 9.5 Hz, 2H), 2.29 (s, 3H), 1.29 (t, J = 7.2 Hz, 3H). | 517.2 |
| 21FF | 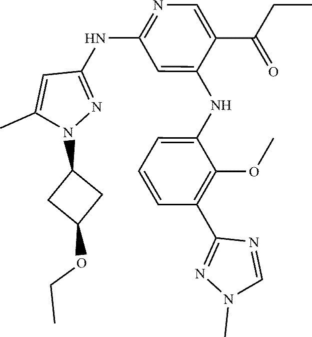 [SMILES: CCO[C@H]1C[C@@H](n2nc(Nc3cc(Nc4cccc(-c5ncn(C)n5)c4OC)c(C(=O)CC)cn3)cc2C)C1] | 1H NMR (400 MHz, Methanol-d4) δ 8.70 (s, 1H), 8.47 (s, 1H), 7.70 - 7.60 (m, 2H), 7.36 (dd, J = 14.5, 6.6 Hz, 2H), 5.86 (s, 1H), 4.35 - 4.22 (m, 1H), 4.02 (s, 3H), 3.88 - 3.76 (m, 1H), 3.74 (s, 3H), 3.41 (q, J = 7.0 Hz, 2H), 3.03 (q, J = 7.4 Hz, 2H), 2.70 - 2.60 (m, 2H), 2.46 - 2.37 (m, 2H), 2.21 (s, 3H), 1.23 (t, J = 7.3 Hz, 3H), 1.12 (t, J = 7.0 Hz, 3H). | 531.3 |
| 21GG | 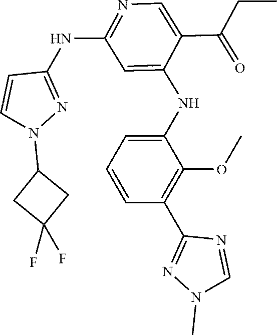 [SMILES: CCC(=O)c1cnc(Nc2ccn(C3CC(F)(F)C3)n2)cc1Nc1cccc(-c2ncn(C)n2)c1OC] | 1H NMR (400 MHz, Methanol-d4) δ 8.74 (s, 1H), 8.49 (s, 1H), 7.71 - 7.60 (m, 2H), 7.56 (d, J = 2.4 Hz, 1H), 7.33 - 7.29 (m, 2H), 6.11 (d, J = 2.4 Hz, 1H), 4.70 - 4.66 (m, 1H), 4.04 (s, 3H), 3.74 (s, 3H), 3.11 -2.94 (m, 6H), 1.26 (t, J = 7.3 Hz, 3H). | 509.2 |

### Example 22

Preparation of N-(4-((2-methoxy-3-(1-methyl-1H-1,2,4-tliazol-3-yl)phenyl)amino)-5-propionylpyridin-2-yl)-2-(1-methyl-1H-pyrazol-4-yl)cyclopropane-1-carboxamide (Compound 22A)

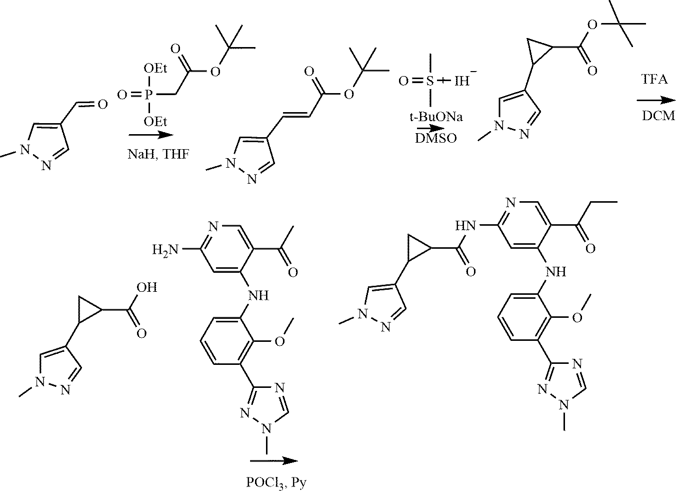

### Step 1: Tert-butyl (E)-3-(1-methyl-1H-pyrazol-4-yl)acrylate

To a stirred solution of tert-butyl 2-diethoxyphosphorylacetate (1.51 g, 6.0 mmol) in tetrahydrofuran (15 mL) was added sodium hydride (131 mg, 5.45 mmol) in portions at 0° C. and stirred for 30 min at 0° C. Then 1-methylpyrazole-4-carbaldehyde (600 mg, 5.45 mmol) was added at 0° C., the cooling bath was removed, and the solution was stirred for 1 hour at room temperature under a nitrogen atmosphere. The reaction was quenched by the addition of saturated sodium bicarbonate (30 mL), then the mixture was extracted with ethyl acetate (3 × 30 ml). The combined organic layers were concentrated under vacuum to afford tert-butyl (E)-3-(1-methyl-1H-pyrazol-4-yl)acrylate (800 mg, 71%) as an oil that was carried onto the next step without further purification.

### Step 2: Tert-Butyl 2-(1-methyl-1H-pyrazol-4-yl)cyc1opropane-1-carboxylate

A solution of trimethylsulfoxonium iodide (687 mg, 3.1 mmol) and potassium tert-butoxide (300 mg, 3.1 mmol) in dimethylsulfoxide (10 mL) was stirred under a nitrogen atmosphere for 30 minutes at 0° C. Then tert-butyl (E)-3-(1-methyl-1H-pyrazol-4-yl)acrylate (500 mg, 2.40 mmol) was added, the cooling bath was removed, and the mixture was stirred at room temperature for 1 hour. The product and the hydrolysis product of the next step (M+1=167) were both detected by LCMS. The mixture was extracted with ethyl acetate (3 × 10 mL), the combined organic layers were concentrated under vacuum. Then the residue was purified by Prep-TLC (PE:EA=1:1) to afford tert-butyl 2-(1-methylpyrazol-4-yl)cyclopropanecarboxylate (180 mg, 0.81 mmol, 33.73% yield) as a yellow oil. The water phase was acidified to Ph=2-3 with HCl(aq.) (1 M), and the mixture were extracted with DCM (3×10mL). The combined organic layers were concentrated under vacuum. Then the residue was purified by Prep-TLC (DCM: methanol=5:1) to afford 2-(1-methylpyrazol-4-yl)cyclopropanecarboxylic acid (170 mg, 1.02 mmol, 42.61% yield) as a yellow oil the hydrolysis product of the next step.

### Step 3: 2-(1-methyl-1H-pyrazol-4-yl)cyclopropane-1-carboxylic Acid

To a stirred solution of tert-butyl 2-(1-methyl-1H-pyrazol-4-yl)cyclopropane-1-carboxylate (180 mg, 0.81 mmol) in dichloromethane (5 mL) was added trifluoroacetic acid (1 mL) dropwise at 0° C. under nitrogen atmosphere. The resulting solution was stirred for 2 hours at room temperature. The mixture was concentrated under vacuumand the residue was purified by Prep-TLC (DCM:methanol 5:1) to yield 2-(1-methyl-1H-pyrazol-4-yl)cyclopropane-1-carboxylic acid (130 mg, 97% yield) as an oil.

### Step 4: N-(4-((2-methoxy-3-(1-methyl-1H-1,2,4-triazol-3-yl)phenyl)amino)-5-propionylpyridin-2-yl)-2-(1-methyl-U/-pyrazol-4-yl)cyclopropane-1-carboxamide

To a stirred solution of 2-(1-methyl-1H-pyrazol-4-yl)cyclopropane-1-carboxylic acid (100 mg, 0.60 mmol) and 1-(6-amino-4-((2-methoxy-3-(1-methyl-1H-1,2,4-triazol-3-yl)phenyl)amino)pyridin-3-yl)propan-1-one (106 mg, 0.30 mmol) in pyridine (5 mL) was added phosphorus oxychloride (461 mg, 3.0 mmol) dropwise at 0° C. under a nitrogen atmosphere. The solution was stirred for 30 minutes at 0° C. The mixture was concentrated under vacuum, purified by Prep-HPLC to yield N-(4-((2-methoxy-3-(1-methyl-1H-1,2,4-triazol-3 -yl)phenyl)amino)-5-propionylpyridin-2-yl)-2-(1-methyl-1H-pyrazol-4-yl)cyclopropane-1-carboxamide (12.4 mg, 8% yield). ¹H-NMR (CD₃OD, 400 MHz, ppm): 8.80 (s, 1H), 8.59 (s, 1H), 7.91 (d, J=7.8 Hz, 1H), 7.56 (d, J=7.9 Hz, 1H), 7.49 (s, 1H), 7.40 (t, J=7.9 Hz, 1H), 7.35 (s, 1H), 6.56 (s, 1H), 4.05 (s, 3H), 3.83 (s, 3H), 3.71 (s, 3H), 3.20-3.16 (m, 2H), 2.52-2.45 (m, 1H), 1.92-1.83 (m, 1H), 1.70-1.60 (m, 1H), 1.45-1.42 (m, 1H), 1.28 (q, J= 7.2 Hz, 3H).

$$\text{(ES,} m / z ) : {[ \text{M+H]}}^{+} 501.15.$$

### Preparation of Compounds 22B-22N

Compounds 22B-22N, as indicated in TABLE 16, were prepared in a similar manner and according to the general synthetic schemes and procedures described herein.

TABLE 16

COMPOUNDS 22B THROUGH 22N

| Cmpd. No. | Structure | ¹H NMR | MS (M+H)⁺ |
|---|---|---|---|
| 22B | 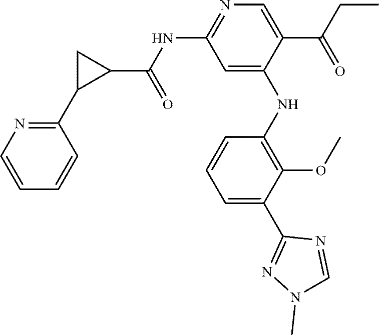 [SMILES: CCC(=O)c1cnc(NC(=O)C2CC2c2ccccn2)cc1Nc1cccc(-c2ncn(C)n2)c1OC] | 1H NMR (400 MHz, DMSO-d6) δ 11.06 (s, 1H), 10.97 (s, 1H), 8.87 (m, 1H), 8.58 (m, 1H), 8.45 (m, 1H), 8.02 (m, 1H), 7.70 (m, 2H), 7.57 (m, 1H), 7.43 (m, 1H), 7.32 (m, 1H), 7.23 (m, 2H), 3.96 (s, 3H), 3.72 (s, 3H), 3.14 (m, 2H), 2.56 (m, 2H), 1.5 (m, 2H), 1.13 (m, 3H). | 498.30 |
| 22C | 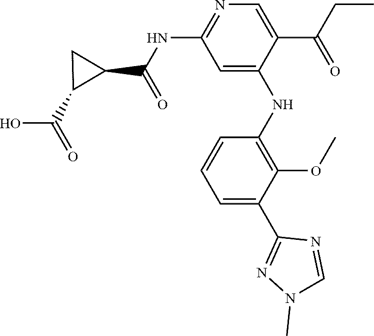 [SMILES: CCC(=O)c1cnc(NC(=O)[C@@H]2C[C@H]2C(=O)O)cc1Nc1cccc(-c2ncn(C)n2)c1OC] | 1H NMR (300 MHz, DMSO-d6) δ 11.24 (s, 1H), 11.07 (s, 1H), 8.86 (m, 1H), 8.57 (m, 1H), 7.69-7.66 (m, 2H), 7.50 (m, 1H), 7.27 (m, 1H), 3.93 (s, 3H), 3.69 (s, 3H), 3.13 (m, 2H), 2.44 (m, 1H), 1.87 (m, 1H), 1.32 (m, 2H), 1.13 (m, 3H). | 465.25 |
| 22D | 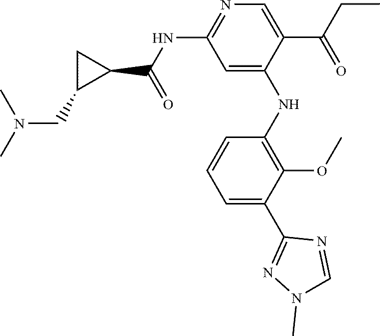 [SMILES: CCC(=O)c1cnc(NC(=O)[C@@H]2C[C@H]2CN(C)C)cc1Nc1cccc(-c2ncn(C)n2)c1OC] | 1H NMR (300 MHz, Methanol-d4) δ 8.83 (m, 1H), 8.54 (m, 1H), 7.86 (m, 1H), 7.57 (m, 1H), 7.38 (m, 1H), 7.02 (m, 1H), 4.05 (s, 3H), 3.72 (s, 3H), 3.3-3.15 (m, 4H), 2.94 (s, 6H), 2.00 (m, 1H), 1.87 (m, 1H), 1.47 (m, 1H), 1.3-1.15 (m, 4H). | 478.20 |
| 22E | 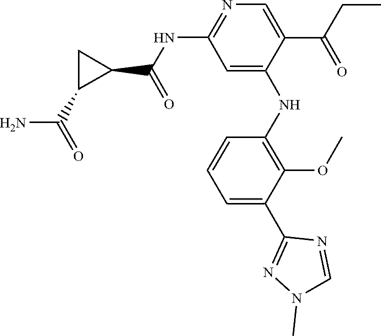 [SMILES: CCC(=O)c1cnc(NC(=O)[C@@H]2C[C@H]2C(N)=O)cc1Nc1cccc(-c2ncn(C)n2)c1OC] | 1H NMR (300 MHz, DMSO-d6) δ 11.04 (m, 2H), 8.88 (m, 1H), 8.57 (m, 1H), 7.93 (m, 1H), 7.66 (m, 2H), 7.52 (m, 1H), 7.26 (m, 1H), 7.01 (m, 1H), 3.95 (s, 3H), 3.71 (s, 3H), 3.12 (m, 2H), 2.35 (m, 1H), 2.03 (m, 1H), 1.15 (m, 5H). | 464.25 |
| 22F | 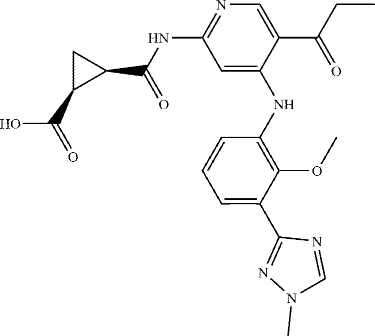 [SMILES: CCC(=O)c1cnc(NC(=O)[C@@H]2C[C@@H]2C(=O)O)cc1Nc1cccc(-c2ncn(C)n2)c1OC] | 1H NMR (300 MHz, Methanol-d4) δ 8.80 (m, 1H), 8.47 (m, 1H), 8.01 (m, 1H), 7.63 (m, 2H), 7.27 (m, 1H), 4.01 (s, 3H), 3.70 (s, 3H), 3.09 (m, 2H), 2.31 (m, 1H), 2.11 (m, 1H), 1.59 (m, 1H), 1.32-1.18 (m, 4H). | 465.15 |
| 22G | 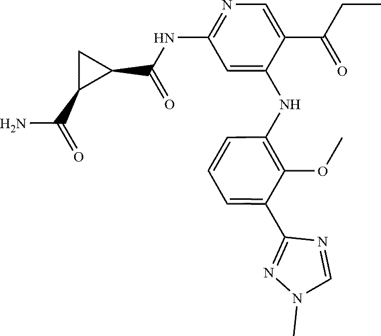 [SMILES: CCC(=O)c1cnc(NC(=O)[C@@H]2C[C@@H]2C(N)=O)cc1Nc1cccc(-c2ncn(C)n2)c1OC] | 1H NMR (300 MHz, Methanol-d4) δ 8.78 (m, 1H), 8.58 (m, 1H), 7.91 (m, 1H), 7.57 (m, 1H), 7.41 (m, 1H), 6.58 (m, 1H), 4.05 (s, 3H), 3.73 (s, 3H), 3.15 (m, 2H), 2.23 (m, 2H), 1.68 (m, 1H), 1.32 (m, 1H), 1.26 (m, 3H). | 464.10 |
| 22H | 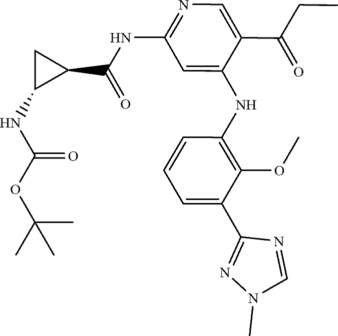 [SMILES: CCC(=O)c1cnc(NC(=O)[C@@H]2C[C@H]2NC(=O)OC(C)(C)C)cc1Nc1cccc(-c2ncn(C)n2)c1OC] | 1H NMR (300 MHz, DMSO-d6) δ 11.00 (m, 1H), 8.92 (s, 1H), 8.57 (s, 1H), 8.06 (s, 1H), 7.65 (m, 1H), 7.54 (m, 2H), 7.26 (m, 1H), 6.15 (m, 1H), 3.95 (s, 3H), 3.73 (s, 3H), 3.13 (m, 2H), 2.78 (m, 1H), 2.35 (m, 2H), 1.82 (m, 1H), 1.35 (s, 9H), 1.12 (m, 3H). | 536.25 |
| 22I | 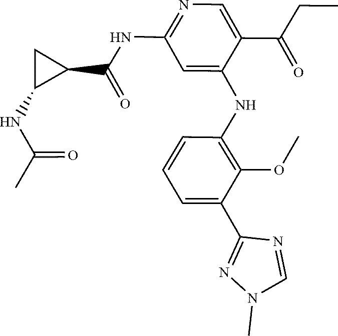 [SMILES: CCC(=O)c1cnc(NC(=O)[C@@H]2C[C@H]2NC(C)=O)cc1Nc1cccc(-c2ncn(C)n2)c1OC] | 1H NMR (300 MHz, Methanol-d4) δ 8.81 (s, 1H), 8.44 (s, 1H), 8.00 (m, 1H), 7.55 (m, 2H), 7.18 (m, 1H), 6.32 (m, 1H), 3.92 (s, 3H), 3.62 (s, 3H), 3.04 (m, 2H), 2.79 (m, 1H), 2.38 (m, 2H), 1.92 (m, 1H), 1.76 (s, 3H), 1.13 (m, 3H). | 478.30 |
| 22J | 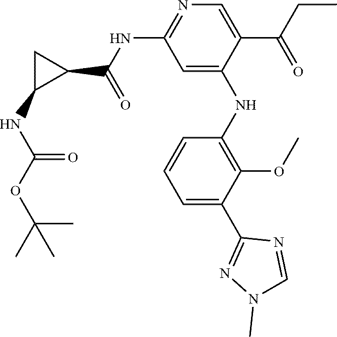 [SMILES: CCC(=O)c1cnc(NC(=O)[C@@H]2C[C@@H]2NC(=O)OC(C)(C)C)cc1Nc1cccc(-c2ncn(C)n2)c1OC] | 1H NMR (300 MHz, Methanol-d4) δ 8.93 (m, 1H), 8.49 (m, 1H), 8.00 (m, 1H), 7.65 (m, 2H), 7.28 (m, 1H), 6.16 (m, 1H), 4.03 (s, 3H), 3.73 (s, 3H), 3.14 (m, 2H), 2.88 (m, 1H), 2.50 (m, 2H), 2.06 (m, 1H), 1.40 (s, 9H), 1.24 (m, 3H). | 536.35 |
| 22K | 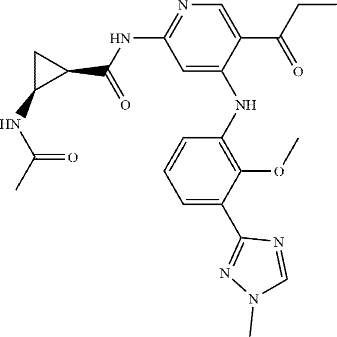 [SMILES: CCC(=O)c1cnc(NC(=O)[C@@H]2C[C@@H]2NC(C)=O)cc1Nc1cccc(-c2ncn(C)n2)c1OC] | 1H NMR (300 MHz, DMSO-d6) δ 11.01 (s, 1H), 8.95 (s, 1H), 8.6-8.5 (m, 2H), 8.17 (s, 1H), 7.68 (m, 1H), 7.56 (m, 1H), 7.28 (m, 1H), 6.35 (m, 1H), 3.97 (s, 3H), 3.74 (s, 3H), 3.15 (m, 2H), 2.78 (m, 1H), 2.4-2.3 (m, 2H), 1.85 (m, 1H), 1.75 (s, 3H), 1.13 (m, 3H). | 478.30 |
| 22L | 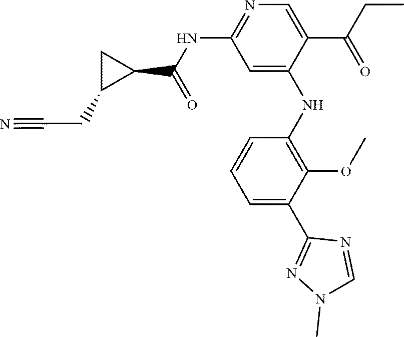 [SMILES: CCC(=O)c1cnc(NC(=O)[C@@H]2C[C@H]2CC#N)cc1Nc1cccc(-c2ncn(C)n2)c1OC] | 1H NMR (300 MHz, DMSO-d6) δ 11.02 (s, 1H), 8.99 (s, 1H), 8.57 (s, 2H), 8.22 (s, 1H), 7.66 (m, 1H), 7.55 (m, 1H), 7.27 (m, 1H), 4.91 (m, 1H), 3.95 (s, 3H), 3.73 (s, 3H), 3.15 (m, 4H), 2.85 (m, 1H), 2.35 (m, 2H), 1.94 (m, 1H), 1.13 (m, 3H). | 460.15 |
| 22M | 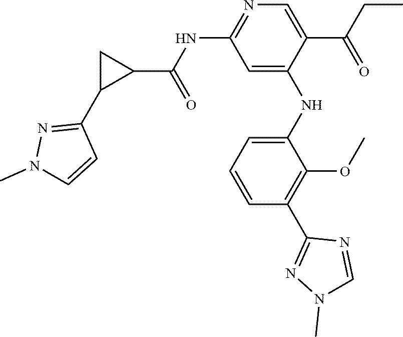 [SMILES: CCC(=O)c1cnc(NC(=O)C2CC2c2ccn(C)n2)cc1Nc1cccc(-c2ncn(C)n2)c1OC] | 1H NMR (400 MHz, Methanol-d4) δ 8.84 (m, 1H), 8.50 (m, 1H), 8.13 (m, 1H), 7.67 (m, 2H), 7.47 (m, 1H), 7.32 (m, 1H), 6.08 (m, 1H), 4.04 (s, 3H), 3.83 (s, 3H), 3.74 (s, 3H), 3.12 (m, 2H), 2.47 (m, 1H), 2.20 (m, 1H), 1.64 (m, 1H), 1.36 (m, 1H), 1.25 (m, 3H). | 501.30 |
| 22N | 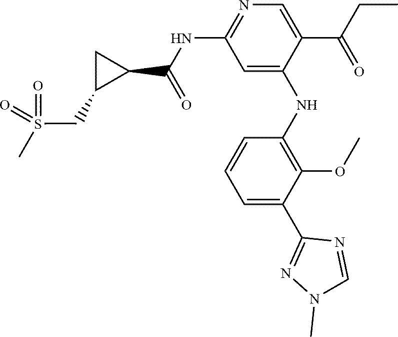 [SMILES: CCC(=O)c1cnc(NC(=O)[C@@H]2C[C@H]2CS(C)(=O)=O)cc1Nc1cccc(-c2ncn(C)n2)c1OC] | 1H NMR (300 MHz, Methanol-d4) δ 8.85 (m, 1H), 8.49 (m, 1H), 8.09 (m, 1H), 7.65 (m, 2H), 7.30 (m, 2H), 4.04 (s, 3H), 3.73 (s, 3H), 3.25-3.07 (m, 4H), 3.00 (m, 3H), 2.02 (m, 1H), 1.75 (m, 1H), 1.34 (m, 1H), 1.25 (m, 3H), 1.10 (m, 1H). | 513.25 |

### Example 23

Preparation of N-(4-((2-methoxy-3-(1-methyl-1H-12,4-triazol-3-yl)phenyl)amino)-5-propionylpyridin-2-yl)-2-(1-methylpiperidin-4-yl)acetamide (Compound 23A)

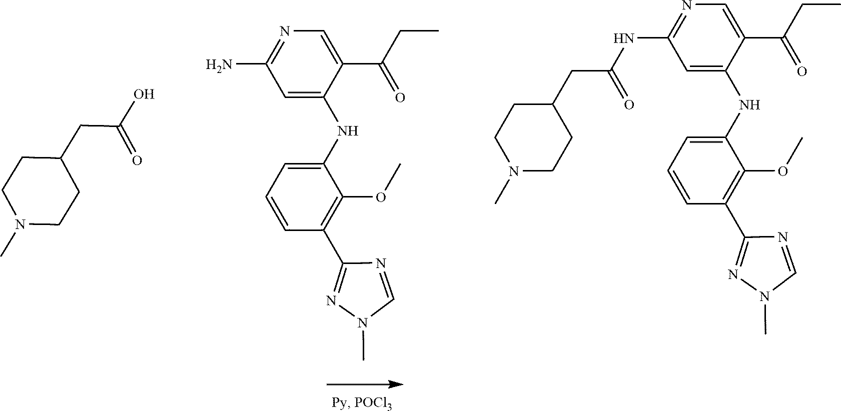

Under an atmosphere of nitrogen was placed 2-(1-methyl-4-piperidyl)acetic acid (45 mg, 0.29 mmol), 1-(6-amino-4-((2-methoxy-3-(1-methyl-1H-1,2,4-triazol-3-yl)phenyl)amino)pyridin-3-yl)propan-1-one (50 mg, 0.14 mmol) in pyridine (3 mL), cooled to 0° C., then added phosphorus oxychloride (110 mg, 0.72 mmol) slowly. The solution was stirred for 15 minutes at 0° C., concentrated under vacuum, diluted with water (10 ml) and extracted with dichloromethane (3 × 10 mL). The extracts were dried with anhydrous sodium sulfate, concentrated under vacuum, and purified by Prep-HPLC to yield N-(4-((2-methoxy-3-(1-methyl-1H-1,2,4-triazol-3-yl)phenyl)amino)-5-propionylpyridin-2-yl)-2-(1-methylpiperidin-4-yl)acetamide (23.5 mg, 33%) as a solid. ¹H-NMR (Methanol-d4, 300 MHz, ppm): 8.84 (s, 1H), 8.49 (s, 1H), 8.12 (s, 1H), 7.62-7.71 (m, 2H), 7.32 (t, J= 7.9 Hz, 1H), 4.04 (s, 3H), 3.74 (s, 3H), 3.11 (m, 2H), 2.89 (d, J= 11.6 Hz, 2H), 2.37 (d, J= 7.0 Hz, 2H), 2.29 (s, 3H), 2.14-2.01 (m, 2H), 1.90-1.74 (m, 3H), 1.47-1.29 (m, 2H), 1.25 (t, J= 7.3 Hz, 3H).

$$\text{(ES,} m / z ) : {[ \text{M+H]}}^{+} 492.30.$$

### Preparation of Compounds 23B-23H

Compounds 23B-23H, as indicated in TABLE 17, were prepared in a similar manner and according to the general synthetic schemes and procedures described herein.

TABLE 17

COMPOUNDS 23B THROUGH 23H

| Cmpd. No. | Structure | ¹H NMR | MS (M+H)⁺ |
|---|---|---|---|
| 23B | 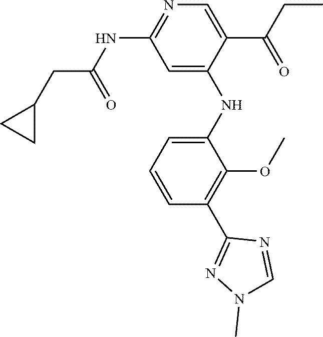 [SMILES: CCC(=O)c1cnc(NC(=O)CC2CC2)cc1Nc1cccc(-c2ncn(C)n2)c1OC] | 1H NMR (300 MHz, DMSO-d6) δ 11.06 (s, 1H), 10.47 (s, 1H), 8.88 (m, 1H), 8.57 (m, 1H), 8.08 (m, 1H), 7.66 (m, 1H), 7.56 (m, 1H), 7.29 (m, 1H), 3.96 (s, 3H), 3.73 (s, 3H), 3.14 (m, 2H), 2.26 (m, 2H), 1.13 (m, 3H) 1.00 (m, 1H), 0.46 (m, 2H), 0.18 (m, 2H). | 435.30 |
| 23C | 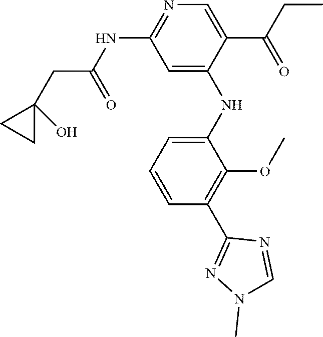 [SMILES: CCC(=O)c1cnc(NC(=O)CC2(O)CC2)cc1Nc1cccc(-c2ncn(C)n2)c1OC] | 1H NMR (400 MHz, Methanol-d4) δ 8.82 (m, 1H), 8.47 (m, 1H), 8.06 (m, 1H), 7.65 (m, 2H), 7.31 (m, 1H), 4.01 (s, 3H), 3.72 (s, 3H), 3.10 (m, 2H), 2.60 (m, 2H), 1.24 (m, 4H), 1.04 (m, 3H). | 451.25 |
| 23D | 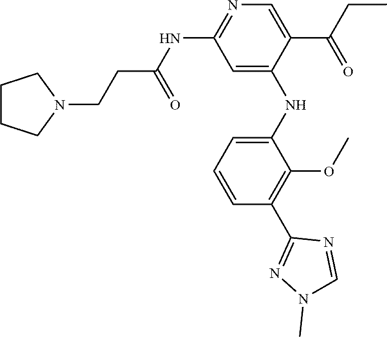 [SMILES: CCC(=O)c1cnc(NC(=O)CCN2CCCC2)cc1Nc1cccc(-c2ncn(C)n2)c1OC] | 1H NMR (300 MHz, Methanol-d4) δ 8.84 (m, 1H), 8.47 (m, 1H), 8.10 (m, 1H), 7.65 (m, 2H), 7.28 (m, 1H), 4.01 (s, 3H), 3.72 (s, 3H), 3.10 (m, 2H), 2.85 (m, 2H), 2.61 (m, 6H), 1.83 (m, 4H), 1.24 (m, 3H). | 478.35 |
| 23E | 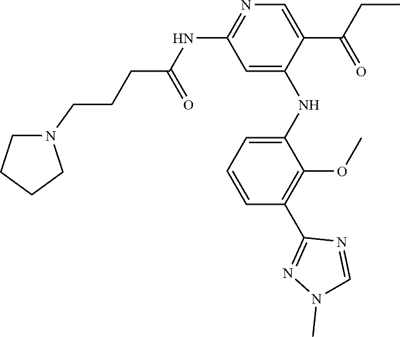 [SMILES: CCC(=O)c1cnc(NC(=O)CCCN2CCCC2)cc1Nc1cccc(-c2ncn(C)n2)c1OC] | 1H NMR (300 MHz, DMSO-d6) δ 11.04 (s, 1H), 10.58 (s, 1H), 8.85 (m, 1H), 8.55 (m, 1H), 8.04 (m, 1H), 7.64 (m, 1H), 7.53 (m, 1H), 7.26 (m, 1H), 3.93 (s, 3H), 3.70 (s, 3H), 3.11 (m, 2H), 2.49-2.34 (m, 8H), 1.65 (m, 6H) 1.12 (m, 3H) | 492.35 |
| 23F | 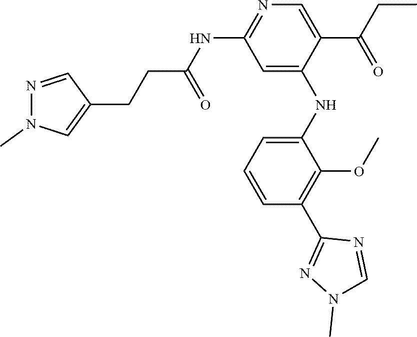 [SMILES: CCC(=O)c1cnc(NC(=O)CCc2cnn(C)c2)cc1Nc1cccc(-c2ncn(C)n2)c1OC] | ¹H NMR (300 MHz, Methanol-d4) δ 8.80 (m, 1H), 8.47 (m, 1H), 8.08 (m, 1H), 7.65 (m, 2H), 7.38 (m, 1H), 7.31 (m, 2H), 4.02 (s, 3H), 3.84 (s, 3H), 3.72 (s, 3H), 3.12 (m, 2H), 2.80 (m, 2H), 2.66 (m, 2H), 1.24 (m, 3H). | 489.25 |
| 23G | 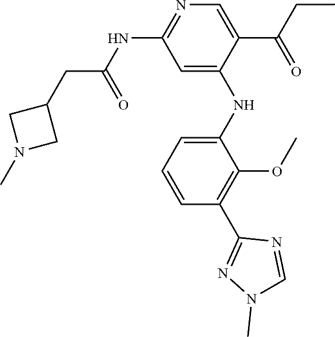 [SMILES: CCC(=O)c1cnc(NC(=O)CC2CN(C)C2)cc1Nc1cccc(-c2ncn(C)n2)c1OC] | 1H NMR (300 MHz, Methanol-d4) δ 11.22 (s, 1H), 8.68 (m, 1H), 8.56 (m, 1H), 7.80 (m, 1H), 7.60 (m, 1H), 7.34 (m, 1H), 4.42 (m, 1H), 4.05-4.00 (m, 4H), 3.74-3.65 (m, 4H), 3.36 (m, 1H), 3.11-3.02 (m, 5H), 2.75 (m, 1H), 2.42 (m, 2H), 1.22 (m, 3H). | 464.10 |
| 23H | 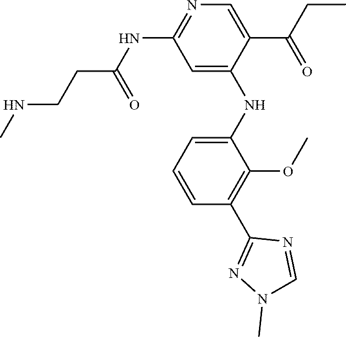 [SMILES: CCC(=O)c1cnc(NC(=O)CCNC)cc1Nc1cccc(-c2ncn(C)n2)c1OC] | 1H NMR (400 MHz, Methanol-d4) δ 8.81 (m, 1H), 8.47 (m, 1H), 8.10 (m, 1H), 7.64 (m, 1H), 7.29 (m, 1H), 4.02 (s, 3H), 3.71 (s, 3H), 3.11 (m, 2H), 2.88 (m, 2H), 2.63 (m, 2H), 2.40 (s, 3H), 1.23 (m, 3H). | 438.15 |

### Example 24

Preparation of 1-(4-((2-methoxy-3-(1-methyl-1H-1,2,4-triazol-3-yl)phenyl)amino)-6-((5-(2-methoxyethoxy)-1-methyl-1H-pyrazol-3-yl)amino)pyridin-3-yl)propan-1-one (Compound 24A)

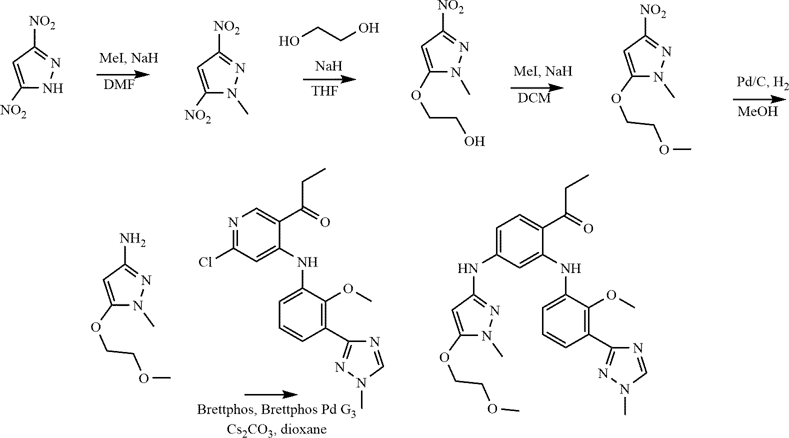

### Step 1: 1-methyl-3,5-dinitro-1H-pyrazole

To a solution of 3,5-dinitro-1H-pyrazole (3.00 g, 19.0 mmol) in N,N-dimethylformamide (30 mL) under a nitrogen atmosphere at 0° C. was added sodium hydride (1.14 g, 28.5 mmol, 60% dispersion in mineral oil). Stirred for 30 minutes at 0° C., then added iodomethane (8.08 g, 56.9 mmol). The mixture was stirred for 2 hours at 20°, quenched with saturated ammonium chloride (150 mL) and extracted with ethyl acetate (100 mL × 3). The combined extracts were washed with water (150 mL × 5), brine (100 mL), dried over anhydrous sodium sulfate and filtered. The filtrate was evaporated to dryness to afford crude 1-methyl-3,5-dinitro-1H-pyrazole (2.42 g, 74%) as a solid that was carried on without further purification.

### Step 2: 2-((1-methyl-3-nitro-1H-pyrazol-5-yl)oxy)ethan-1-ol

To a solution of ethylene glycol (8 mL) in tetrahydrofuan (16 mL) was added sodium hydride (223 mg, 5.6 mmol, 60% dispersion in mineral oil) at 0° C. Stirred for 30 minutes at 0° C. then added 1-methyl-3,5-dinitro-1H-pyrazole (1.00 g, 5.8 mmol). After stirring for 16 hours at 70° C., the reaction mixture was cooled to room temperature, poured into ice-water (100 mL) and a white precipitate formed. The solid was collected by filtration, washed with water (50 mL × 3), and dried under vacuum to afford 2-((1-methyl-3-nitro-1H-pyrazol-5-yl)oxy)ethan-1-ol (740 mg, 68%) as a solid.

### Step 3: 5-(2-methoxyethoxy)-1-methyl-3-nitro-1H-pyrazole

To a solution of 2-((1-methyl-3-nitro-1H-pyrazol-5-yl)oxy)ethan-1-ol (200 mg, 1.07 mmol) in N,N-dimethylformamide (5 mL) was added sodium hydride (64 mg, 1.6 mmol, 60% dispersion in mineral oil) at 0° C. Stirred for 30 minutes at 0° C., then added iodomethane (758 mg, 5.3 mmol). Stirred at 0° C. for 2 hours, quenched with saturated ammonium chloride (50 mL) and extracted ethyl acetate (50 mL × 3). The combined organic layers were washed with water (50 mL × 5) and brine (50 mL), dried over anhydrous sodium sulfate, filtered and concentrated to yield 5-(2-methoxyethoxy)-1-methyl-3-nitro-1H-pyrazole (200 mg) as a solid which was carried further without purification.

### St-ep 4: 5-(2-methoxyethoxy)-1-methyl-1H-pyrazol-3-amine

To a solution of 5-(2-methoxyethoxy)-1-methyl-3-nitro-1H-pyrazole (200 mg, 1.0 mmol) in methanol (10 mL) was added 10% palladium on carbon (11 mg) under nitrogen atmosphere. The resulting mixture was degassed and backfilled with hydrogen three times and stirred for 1 hour at 20° C. under hydrogen atmosphere (1 atm.). The mixture was filtered over Celite, washed with methanol (10 mL × 3), and concentrated to yield 5-(2-methoxyethoxy)-1-methyl-1H-pyrazol-3-amine (160 mg, 94%) as a solid.

### Step 5: 1-(4-((2-methoxy-3-(1-methyl-1H-L2,4-triazol-3-yl)phenyl)amino)-6-((5-(2-methoxyethoxy)-l-methyl-IH-pyrazol-3-yl)amino)pyridin-3-yl)propan-1-one

A mixture of 5-(2-methoxyethoxy)-1-methyl-1H-pyrazol-3-amine (101 mg, 0.6 mmol), 1-(6-chloro-4-((2-methoxy-3-(1-methyl-1H-1,2,4-triazol-3-yl)phenyl)amino)pyridin-3-yl)propan-1-one (200 mg, 0.5 mmol), BrettPhos (29 mg, 0.05 mmol), cesium carbonate (351 mg, 1.1 mmol) and BrettPhos-Pd-G3 (24 mg, 0.03 mmol) in 1,4-dioxane (10 mL) was stirred at 90° C. overnight under a nitrogen atmosphere. The mixture was cooled to room temperature, diluted with water (50 mL) and extracted with ethyl acetate (100 mL × 3). The combined organic layers were washed with water (50 mL × 3) and brine (50 mL), dried over anhydrous sodium sulfate, filtered and concentrated under reduced pressure. The residue was purified by prep-HPLC to give 1-(4-((2-methoxy-3-(1-methyl-1H-1,2,4-triazol-3-yl)phenyl)amino)-6-((5-(2-methoxyethoxy)-1-methyl-1H-pyrazol-3-yl)amino)pyridin-3-yl)propan-1-one (39.4 mg, 14%) as a solid. ¹H NMR (400 MHz, DMSO-d6) δ 11.13 (s, 1H), 9.53 (s, 1H), 8.77 (s, 1H), 8.57 (s, 1H), 7.69 - 7.5(m, 3H), 7.29 (t, J = 7.9 Hz, 1H), 5.58 (s, 1H), 4.19 - 4.12 (m, 2H), 3.96 (s, 3H), 3.74 (s, 3H), 3.68 - 3.62 (m, 2H), 3.44 (s, 3H), 3.33 - 3.32 (m, 3H), 3.05 (q, J = 7.3 Hz, 2H), 1.13 (t, J = 7.2 Hz, 3H).

$$\text{LC-MS: (ES,} m / z ) : {[ \text{M+H]}}^{+} 507.35.$$

### Example 25

Preparation of N-(4-((5-fluoro-2-methoxy-3-(1-methyl-1H-1,2,4-triazol-3-yl)phenyl)amino)-5-propionylpyridin-2-yl)cyclopropanecarboxamide (Compound 25A)

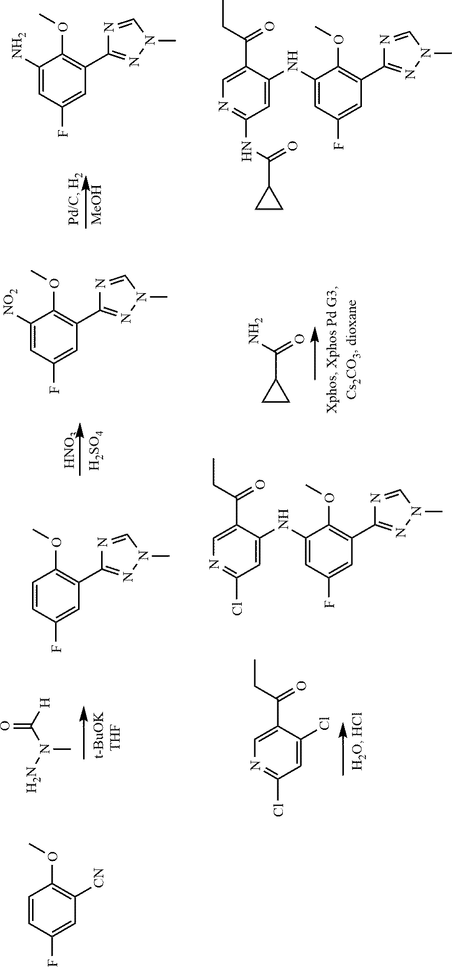

### Step 1: 3-(5-fluoro-2-methoxyphenyl)-1-methyl-1H-1,2,4-triazole

In a round bottom flask maintained with an inert atmosphere of nitrogen was placed methyl 5-fluoro-2-methoxy-benzonitrile (5.00 g, 33.1 mmol) and N-amino-N-methyl-formamide (12.3 g, 165 mmol) in tetrahydrofuran (100 mL). The mixture was cooled to 0° C., added potassium tert-butoxide (14.9 g, 132 mmol) slowly. The solution was stirred at room temperature for 6 hours. The mixture was diluted with water (20 mL), extracted with ethyl acetate (20 ml x 3), dried combined extracts with sodium sulfate, filtered and concentrated under vacuum. The residue was purified by column chromatography eluting with a gradient of 0-100% ethyl acetate in petroleum ether. Desired fractions were combined and concentrated to yield 3-(5-fluoro-2-methoxyphenyl)-1-methyl-1H-1,2,4-triazole (4.30 g, 63%) as a solid.

### Step 2: 3-(5-fluoro-2-methoxy-3-nitrophenl)-1-methyl-1H-1,2,4-triazole

In a round bottom flask maintained with an inert atmosphere of nitrogen was placed 3-(5-fluoro-2-methoxyphenyl)-1-methyl-1H-1,2,4-triazole (4.1 g, 19.8 mmol) and concentrated sulfuric acid (25 mL), cooled to 0° C., then added nitric acid (2.3 mL, 30 mmol, 80 wt%) dropwise. Stirred at 0° C. for 20 hours. Poured mixture into ice water (500 mL), the resulting precipitate was filtrated, washed with water (3 × 50 mL), then dried solids under vacuum to yield 3-(5-fluoro-2-methoxy-3-nitrophenyl)-1-methyl-1H-1,2,4-triazole (3.5 g, 70%) as a solid.

### Step 3: 5-fluoro-2-methoxy-3-(1-methyl-1H-1,2,4-triazol-3-yl)aniline

In a round bottom flask maintained with an inert atmosphere of nitrogen was placed 3-(5-fluoro-2-methoxy-3-nitrophenyl)-1-methyl-1H-1,2,4-triazole (2.0 g, 7.9 mmol) in methanol (40 mL). To the solution was added 10% palladium on carbon (1.5 g) under N₂ atmosphere. Purged and refilled with hyrdrogen, then stirred at room temperature for 2 hours. The mixture was filtered over celite and the filtrates were concentrated under vacuum to afford crude 5-fluoro-2-methoxy-3-(1-methyl-1H-1,2,4-triazol-3-yl)aniline aniline (1.50 g, 85%) as a solid, used directly in the next step without further purification.

### Step 4: 1-(6-chloro-4-((5-fluoro-2-methoxy-3-(1-methyl-1H-1,2,4-triazol-3-yl)phenyl)amino)pyridin-3-yl)propan-1-one

In a round bottom maintained with an inert atmosphere of nitrogen was placed 5-fluoro-2-methoxy-3-(1-methyl-1H-1,2,4-triazol-3-yl)aniline (300 mg, 1.4 mmol), 1-(4,6-dichloropyridin-3-yl)propan-1-one (303 mg, 1.5 mmol), concentrated hydrochloride acid (0.2 mL, 2.8 mmol) and water (10 mL). Stirred at 90° C. overnight. The mixture was cooled to room temperature, basified to pH 7 with sodium bicarbonate solution, and extracted with dichloromethane (20 ml × 3). The extracts were dried with anhydrous sodium sulfate, filtered and concentrated under vacuum. The residue was purified by Prep-TLC (DCM:MeOH=15:1) to afford 1-(6-chloro-4-((5-fluoro-2-methoxy-3 -(1 -methyl- 1H-1,2,4-triazol-3 -yl)phenyl)amino)pyridin-3 -yl)propan-1 -one (320 mg, 62%) as a solid.

### Step 5: N-(4-((5-fluoro-2-methoxy-3-(1-methyl-1H-1,2,4-triazol-3-yl)phenyl)amino)-5-propionylpyridin-2-yl)cyclopropanecarboxamide

In a vial maintained with an inert atmosphere of nitrogen was placed 1-(6-chloro-4-((5-fluoro-2-methoxy-3-(1-methyl-1H-1,2,4-triazol-3-yl)phenyl)amino)pyridin-3-yl)propan-1-one (100 mg, 0.26 mmol), cyclopropanecarboxamide (22 mg, 0.26 mmol), Xphos (24 mg, 0.05 mmol), Xphos Pd G3 (22 mg, 0.026 mmol), cesium carbonate (167 mg, 0.51 mmol) and 1,4-dioxane (5 mL). Heated to 90° C. for 4 hours, then concentrated under vacuum. The residue was purified by Prep-HPLC to yield N-(4-((5-fluoro-2-methoxy-3-(1-methyl-1H-1,2,4-triazol-3-yl)phenyl)amino)-5-propionylpyridin-2-yl)cyclopropanecarboxamide (75 mg, 67%) as a solid. ¹H NMR (300 MHz, Methanol-d4) δ 8.87 (s, 1H), 8.49 (s, 1H), 8.18 (s, 1H), 7.48 - 7.35 (m, 2H), 4.04 (s, 3H), 3.75 (s, 3H), 3.11 (q, J = 7.3 Hz, 2H), 1.90 - 1.81 (m, 1H), 1.24 (t, J = 7.3 Hz, 3H), 1.05 - 0.82 (m, 4H).

$$\text{LC-MS:} \text{(ES,} m / z ) : {[ \text{M+H]}}^{+} 439.20.$$

### Preparation of Compounds 25B-25F

Compounds 25B-25F, as indicated in TABLE 18, were prepared in a similar manner and according to the general synthetic schemes and procedures described herein.

TABLE 18

COMPOUNDS 25B THROUGH 25F

| Cmpd. No. | Structure | ¹H NMR | MS (M+H)⁺ |
|---|---|---|---|
| 25B | 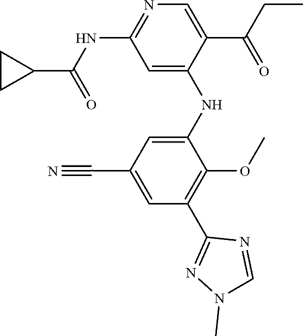 [SMILES: CCC(=O)c1cnc(NC(=O)C2CC2)cc1Nc1cc(C#N)cc(-c2ncn(C)n2)c1OC] | (400 MHz, DMSO-d6) δ 11.13 (s, 1H), 11.00 (s, 1H), 8.92 (s, 1H), 8.64 (s, 1H), 8.05 - 7.97 (m, 3H), 3.98 (s, 3H), 3.78 (s, 3H), 3.14 (q, J = 7.2 Hz, 2H), 2.10 - 2.00 (m, 1H), 1.13 (t, J = 7.2 Hz, 3H), 0.81 (d, J = 6.1 Hz, 4H). | 446.2 |
| 25C | 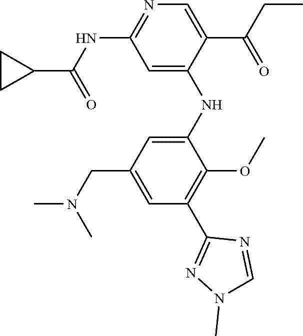 [SMILES: CCC(=O)c1cnc(NC(=O)C2CC2)cc1Nc1cc(CN(C)C)cc(-c2ncn(C)n2)c1OC] | (300 MHz, Methanol-d4) δ 8.86 (s, 1H), 8.57 (s, 1H), 7.90 (d, J = 2.2 Hz, 1H), 7.79 (d, J = 2.2 Hz, 1H), 7.53 (s, 1H), 4.40 (s, 2H), 4.06 (s, 3H), 3.78 (s, 3H), 3.15 (q, J = 7.2 Hz, 2H), 2.96 (s, 6H), 1.90 - 1.86 (m, 1H), 1.26 (t, J = 7.2 Hz, 3H), 1.05 - 0.99 (m, 4H). | 478.2 |
| 25D | 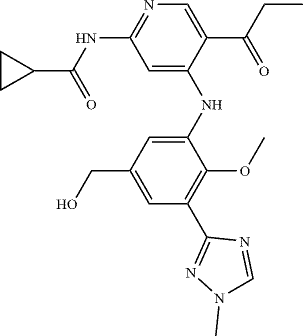 [SMILES: CCC(=O)c1cnc(NC(=O)C2CC2)cc1Nc1cc(CO)cc(-c2ncn(C)n2)c1OC] | (300 MHz, Methanol-d4) δ 8.84 (s, 1H), 8.49 (s, 1H), 8.05 (s, 1H), 7.67 (s, 1H), 7.58 (s, 1H), 4.67 (s, 2H), 4.03 (s, 3H), 3.72 (s, 3H), 3.11 (q, J = 7.3 Hz, 2H), 1.98 - 1.78 (m, 1H), 1.25 (t, J = 7.3 Hz, 3H), 1.02 - 0.72 (m, 4H). | 451.1 |
| 25E | 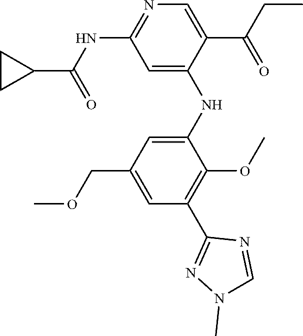 [SMILES: CCC(=O)c1cnc(NC(=O)C2CC2)cc1Nc1cc(COC)cc(-c2ncn(C)n2)c1OC] | (300 MHz, Methanol-d4) δ 8.83 (s, 1H), 8.49 (s, 1H), 8.07 (s, 1H), 7.64 (dd, J = 23.1, 2.1 Hz, 2H), 4.53 (s, 2H), 4.03 (s, 3H), 3.73 (s, 3H), 3.42 (s, 3H), 3.11 (q, J = 7.3 Hz, 2H), 1.96 - 1.74 (m, 1H), 1.24 (t, J = 7.3 Hz, 3H), 1.02 - 0.78 (m, 4H). | 465.2 |
| 25F | 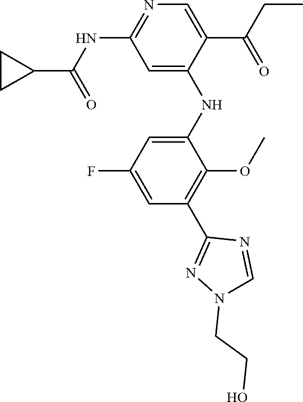 [SMILES: CCC(=O)c1cnc(NC(=O)C2CC2)cc1Nc1cc(F)cc(-c2ncn(CCO)n2)c1OC] | (300 MHz, Methanol-d4) δ 8.87 (s, 1H), 8.54 (s, 1H), 8.19 (s, 1H), 7.48 -7.36 (m, 2H), 4.41 (t, J = 5.1 Hz, 2H), 3.99 (t, J = 5.1 Hz, 2H), 3.77 (s, 3H), 3.12 (q, J = 7.3 Hz, 2H), 1.97 - 1.81 (m, 1H), 1.25 (t, J = 7.3 Hz, 3H), 1.06 - 0.93 (m, 2H), 1.03 - 0.83 (m, 2H). | 469.2 |

### Example 26

Preparation of 1-(6-((1-(cyclopropanecarbonyl)-4,5-dihydro-1H-pyrazol-3-yl)amino)-4-((2-methoxy-3-(1-methyl-1H-1,2,4-triazol-3-yl)phenyl)amino)pyridin-3-yl)propan-1-one (Compound 26A)

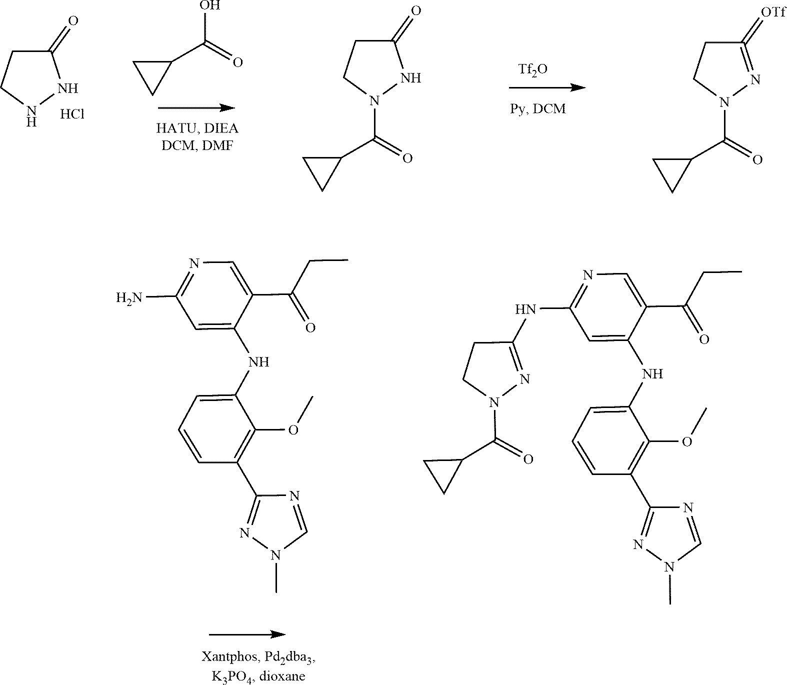

### Step 1: 1-(cyclopropanecarbonyl)pyrazolidin-3-one

In a round bottomed flask was combined pyrazolidin-3-one (700 mg, 8.1 mmol), cyclopropanecarboxylic acid (840 mg, 9.8 mmol), HATU (4.64 g, 12.2 mmol) and N,N-diisopropylethylamine (4.4 mL, 16.3 mmol) in dichloromethane (10 mL) and N,N-dimethylformamide (1 mL). The mixture was stirred at room temperature for 2 hours, then concentrated. The residue was purified by silica gel chromatography eluted with a gradient of 0-10% methanol in dichloromethaneThe fractions containing product were collected and concentrated to yield 1-(cyclopropanecarbonyl)pyrazolidin-3-one (720 mg, 57%) as a solid.

### Step 2: 1-(cyclopropanecarbonyl)-4,5-dihydro-1H-pyrazol-3-yl trifluoromethanesulfonate

To a solution of 1-(cyclopropanecarbonyl)pyrazolidin-3-one (200 mg, 1.3 mmol) and pyridine (0.16 mL, 1.95 mmol) in dichloromethane (10 mL) at -10° C. was added trifluoromethanesulfonic anhydride (730 mg, 2.6 mmol) dropwise at. Stirred at -10° C. for 1 hour, allowed to warm to room temperature. It was subsequently diluted with water (50 ml), extracted with dichloromethane (2 × 50 mL), washed extracts brine (50 mL), dried with anhydrous sodium sulfate, and evaporated. The residue was purified by silica gel chromatography eluting with a gradient of 0-10% methanol in dichloromethane. Desired fractions were combined and concentrated to yield 1-(cyclopropanecarbonyl)-4,5-dihydro-1H-pyrazol-3-yl trifluoromethanesulfonate (150 mg, 40%) as a solid.

### Step 3: 1-(6-((1-(cyclopropanecarbonyl)-4,5-dihydro-1H-pyrazol-3-yl)amino)-4-((2-methoxy-3-(1-methyl-1H-1,2,4-triazol-3-yl)phenyl)amino)pyridin-3-yl)propan-1-one

In a vial was combined 1-(6-amino-4-((2-methoxy-3-(1-methyl-1H-1,2,4-triazol-3-yl)phenyl)amino)pyridin-3-yl)propan-1-one (123.12 mg, 0.35 mmol), 1-(cyclopropanecarbonyl)-4,5-dihydro-1H-pyrazol-3-yl trifluoromethanesulfonate (120 mg, 0.42 mmol eq:1.2), potassium triphosphate (222 mg, 1.05 mmol) XantPhos (40 mg, 0.07 mmol), tris(dibenzylideneacetone)dipalladium(0) (32 mg, 0.035 mmol) in 1,4-dioxane (10 mL) and stirred at 90° C. for 4 hours. The mixture was concentrated and purified by silica gel chromatography eluting with 10% methanol in dichloromethane. The crude product was repurified by Prep-HPLC to yield 1-(6-((1-(cyclopropanecarbonyl)-4,5-dihydro-1H-pyrazol-3-yl)amino)-4-((2-methoxy-3-(1-methyl-1H-1,2,4-triazol-3-yl)phenyl)amino)pyridin-3-yl)propan-1-one (73 mg, 42%) as a solid. ¹H NMR (400 MHz, DMSO-d6) δ 10.90 (s, 1H), 10.04 (s, 1H), 8.84 (s, 1H), 8.56 (s, 1H), 7.62 (d, J = 6.9 Hz, 2H), 7.50 (dd, J = 7.9, 1.6 Hz, 1H), 7.12 (t, J = 7.9 Hz, 1H), 3.95 (s, 3H), 3.75 - 3.65 (m, 5H), 3.11 - 3.01 (m, 4H), 1.75 - 1.65 (m, 1H), 1.13 (t, J = 7.2 Hz, 3H), 0.67 - 0.57 (m, 2H), 0.53 - 0.43 (m, 2H).

$$\text{LC-MS: (ES,} m / z ) : {\text{[M+H]}}^{+} \text{489} \text{.25} \text{.}$$

### Example 27

### TYK2 Activity: TYK2 JH2 NanoBRET® Assay

HEK293T cells were transfected with NanoLuc-TYK2 JH2 Fusion Vector (Promega, customized) using Trans-IT reagent (Mirus, #MIR2700) and incubated for overnight in 37° C. incubator. Cells were harvested using trypLE and resuspended into phenol-free opti-MEM (Life technologies, #11058-021) at 0.25×10⁶/ml. Add 85 µL of cell suspension into white, polypropylene, 96-well plates (Corning, #3600). 90 µL of cells were used for no tracer control samples. 5 µL of diluted NanoBRET K10 tracer (Promega, #CS1810C122) were added to cells to a final concentration of 0.5 µM. Test compounds were diluted into DMSO, and then phenol-free opti-MEM to 10X of final concentration. 10 µL of diluted compounds were added into each well. The cells were incubated with test compound and tracer for 2 hours. 3XNanoBRET® Nano-Glo® Substrate and Extracellular NanoLuc® Inhibitor mixture were prepared and 50 µL of the mixture were added into the wells and mixed. BRET signal was measured using Tecan SPARK plate reader with donor and acceptor emissions at 450 nM and 610 nM, respectively. NanoBRET signal was determined by using the ratio of acceptor signal and donor signal. Binding on TYK2 JH2 domain was calculated by remaining NanoBRET signal relative to DMSO controls and plotted using PRISM (GraphPad) to determine a 50% inhibitory concentration (IC₅₀).

IC₅₀ values are provided for the compounds of the present invention in TABLE 19, below. With respect to TYK2 activity:
“A” denotes an IC₅₀ of less than 5 nM;
“B” denotes an IC₅₀ of from 5 nM to less than 50 nM;
“C” denotes an IC₅₀ of from 50 nM to less than 500 nM; and
“D” denotes an IC₅₀ of 500 nM or more.

TABLE 19

ACTIVITY OF COMPOUNDS 1A THROUGH 7U

| | |
|---|---|
| Compound No. | IC₅₀ (nM) |
| 1A | A |
| 1B | A |
| 1C | A |
| 1D | C |
| 1G | B |
| 1H | B |
| 1I | A |
| 1J | B |
| 1K | A |
| 1L | B |
| 1M | C |
| 1N | C |
| 1O | C |
| 1P | C |
| 2 | D |
| 3 | A |
| 7A | D |
| 7B | D |
| 7C | A |
| 7D | A |
| 7E | A |
| 7F | A |
| 7G | B |
| 7H | A |
| 7I | A |
| 7J | A |
| 7K | A |
| 7L | A |
| 7M | A |
| 7N | A |
| 7O | C |
| 7P | C |
| 7Q | A |
| 7R | A |
| 7S | A |
| 7T | C |
| 7U | A |

### Example 28

### JAK1, JAK2, and JAK3 Activity

Compounds described herein were tested for the ability to inhibit activity of human JAK1, JAK2 and JAK3, which was achieved using TR-FRET assays. Briefly, the Kinases JAK1 (2.5 nM), JAK2 (0.025 nM) and JAK3 (0.0125 nM) were incubated with a series of concentrations of the test compound in the presence of 1 mM JAK Common Substrate (biotin-ahx- EQEDEPEGDYFEWLE-CONH2), 2 nM Eu-labeled anti-pTYRPY20 and 80 nM Streptavidin APC. After 30 min incubation at RT, ATP (30, 5 and 5 mM respectively for JAK1, JAK2 and JAK3) was added to start the reaction, and incubated for 80 min at RT. The reaction was stopped by adding detection buffer and incubated for a further 60 min at RT. The samples were analyzed using Envision to calculate % inhibition at each of the series of concentrations of the test compound. The IC50 value of the compounds for each of the kinases were calculated using XLFit software.

Tyk2 and JAK1, JAK2, and JAK3 IC₅₀ values are provided for compounds 8A-25F in TABLE 20, below. With respect to TYK2 / JAK activity:
“A” denotes an IC₅₀ of less than 5 nM;
“B” denotes an IC₅₀ of from 5 nM to less than 50 nM;
“C” denotes an IC₅₀ of from 50 nM to less than 500 nM; and
“D” denotes an IC₅₀ of 500 nM or more.

TABLE 20

TYK2, JAK1, JAK2 AND JAK3 ACTIVITY - IC₅₀ (nM)

| | | | | |
|---|---|---|---|---|
| Cmpd. No. | TYK2 | JAK1 | JAK2 | JAK3 |
| 8A | A | D | C | C |
| 8B | C | D | D | D |
| 8C | A | D | D | D |
| 8D | A | D | D | D |
| 8E | D | D | D | D |
| 8F | A | D | D | D |
| 8G | A | D | D | D |
| 8H | A | D | D | D |
| 8I | D | D | D | D |
| 8J | A | D | D | D |
| 8K | A | D | D | D |
| 8L | B | D | D | D |
| 8M | A | D | D | D |
| 8N | A | D | D | D |
| 8O | A | D | D | D |
| 9A | A | D | D | D |
| 9B | A | D | D | D |
| 9C | A | D | D | D |
| 9D | B | D | D | D |
| 9E | A | D | D | D |
| 9F | B | D | D | D |
| 9G | A | D | D | D |
| 9H | A | D | D | D |
| 9I | A | D | D | D |
| 9J | A | D | D | D |
| 9K | A | D | D | D |
| 9L | B | D | C | C |
| 9M | A | D | C | D |
| 9N | A | D | D | D |
| 9O | A | D | D | D |
| 9P | A | D | D | D |
| 9Q | A | D | D | D |
| 9R | B | D | D | D |
| 9S | A | D | C | D |
| 9T | A | D | D | D |
| 9U | A | D | D | D |
| 9V | A | D | D | D |
| 9W | A | D | D | D |
| 9X | B | D | D | D |
| 9Y | B | D | D | D |
| 9Z | B | D | C | D |
| 9AA | A | D | C | C |
| 9BB | B | D | D | D |
| 9CC | A | D | D | D |
| 9DD | A | D | D | D |
| 9EE | A | D | D | C |
| 9FF | B | D | D | C |
| 9GG | B | D | D | D |
| 9HH | B | D | D | D |
| 9II | A | D | D | D |
| 9JJ | A | D | D | D |
| 9KK | A | D | D | D |
| 9LL | A | D | D | C |
| 9MM | A | D | D | D |
| 9NN | A | D | D | D |
| 9OO | A | D | C | C |
| 9PP | B | D | D | C |
| 9QQ | B | D | D | C |
| 9RR | A | D | D | D |
| 9SS | A | D | D | D |
| 9TT | A | D | C | C |
| 9UU | A | D | D | D |
| 9VV | A | D | C | C |
| 9WW | B | D | C | C |
| 9XX | A | D | C | C |
| 9YY | A | D | D | C |
| 9ZZ | A | D | D | D |
| 9AAA | A | D | D | D |
| 9BBB | A | D | C | C |
| 9CCC | A | D | D | D |
| 9DDD | A | D | D | D |
| 9EEE | A | D | D | D |
| 9FFF | C | D | D | D |
| 9GGG | B | D | D | D |
| 9HHH | B | D | D | D |
| 9III | B | D | D | D |
| 9JJJ | B | D | D | D |
| 9KKK | A | D | D | D |
| 9LLL | B | D | D | D |
| 9MMM | B | D | D | D |
| 9NNN | B | D | D | D |
| 9OOO | B | D | D | D |
| 9PPP | B | D | C | D |
| 9QQQ | A | D | D | D |
| 9RRR | A | D | D | D |
| 9SSS | A | D | D | D |
| 9TTT | B | D | D | D |
| 9UUU | B | D | D | D |
| 9VVV | B | D | D | D |
| 9XXX | B | D | D | D |
| 9YYY | B | D | D | D |
| 9ZZZ | B | D | D | D |
| 9AAAA | A | D | D | D |
| 9BBBB | A | D | D | D |
| 9CCCC | B | D | D | D |
| 9DDDD | B | D | D | D |
| 9EEEE | B | D | D | D |
| 9FFFF | B | D | D | D |
| 9GGGG | B | D | D | D |
| 9HHHH | A | D | D | D |
| 9IIII | A | D | C | C |
| 9JJJJ | A | D | D | C |
| 9KKKK | A | D | D | C |
| 9LLLL | A | D | D | D |
| 9MMMM | B | D | D | D |
| 9NNNN | A | D | D | D |
| 9OOOO | A | D | D | D |
| 9PPPP | A | D | C | D |
| 9QQQQ | A | D | D | D |
| 9RRRR | B | D | D | D |
| 9SSSS | B | D | D | D |
| 9TTTT | B | D | D | D |
| 9UUUU | C | D | D | D |
| 9WWW | B | D | D | D |
| 10A | A | D | D | D |
| 11A | A | D | D | D |
| 11B | B | D | D | D |
| 11C | A | D | D | D |
| 11D | B | D | D | D |
| 11E | C | D | D | D |
| 11F | A | D | D | D |
| 11G | A | D | D | C |
| 11H | C | D | D | D |
| 11I | B | D | D | D |
| 11J | B | D | D | D |
| 12A | A | D | D | D |
| 13A | A | D | C | C |
| 13B | A | D | D | D |
| 13C | B | D | D | D |
| 13D | A | D | D | D |
| 13D | C | D | D | D |
| 13E | A | D | D | D |
| 13F | B | D | D | D |
| 13G | A | D | D | D |
| 13H | A | D | C | C |
| 13I | A | D | D | D |
| 13J | A | D | D | D |
| 13K | B | D | C | C |
| 13L | D | D | D | D |
| 13M | A | D | C | D |
| 13N | B | D | C | C |
| 130 | A | D | D | D |
| 13P | B | D | D | C |
| 13Q | B | D | D | D |
| 13R | B | D | D | D |
| 13T | B | D | D | D |
| 13U | B | D | D | D |
| 13V | A | D | D | D |
| 13W | C | D | C | C |
| 13X | C | D | C | C |
| 13Y | C | D | D | D |
| 13Z | C | D | D | D |
| 13AA | B | D | D | D |
| 14A | A | D | D | D |
| 15A | A | D | D | D |
| 15B | A | D | D | C |
| 15C | A | D | D | D |
| 15D | A | D | D | D |
| 15E | A | D | D | D |
| 15F | A | D | D | D |
| 15G | A | D | D | D |
| 15H | A | D | D | D |
| 15I | A | D | D | D |
| 15J | A | D | D | D |
| 15K | B | D | D | D |
| 15L | B | D | D | D |
| 15M | B | D | D | D |
| 15N | B | D | D | D |
| 15O | B | D | D | D |
| 15P | A | D | D | D |
| 15Q | A | D | D | D |
| 15R | B | D | D | D |
| 15S | B | D | D | D |
| 15T | A | D | D | D |
| 15U | B | D | D | D |
| 15V | A | D | D | D |
| 15W | B | D | C | C |
| 15X | B | D | C | C |
| 15Y | A | D | C | C |
| 15Z | B | D | D | D |
| 15AA | C | D | D | D |
| 15BB | B | D | D | D |
| 15CC | A | D | D | D |
| 15DD | B | D | D | D |
| 16A | A | D | C | C |
| 17A | A | D | D | D |
| 18A | | | | |
| 18B | A | D | D | D |
| 18C | A | D | D | D |
| 18D | B | D | D | C |
| 18E | A | D | C | C |
| 18F | B | D | D | C |
| 18G | B | D | D | D |
| 18H | B | D | D | D |
| 18I | B | D | D | D |
| 18J | B | D | D | D |
| 18K | B | D | D | D |
| 18L | D | D | C | C |
| 18M | A | D | D | D |
| 18N | A | D | D | D |
| 180 | A | D | D | D |
| 18P | B | D | D | D |
| 18Q | B | D | D | D |
| 18R | A | D | D | C |
| 18S | B | D | D | C |
| 18T | B | D | D | D |
| 18U | A | D | D | D |
| 18V | A | D | D | C |
| 18W | A | D | D | D |
| 18X | A | D | D | C |
| 18Y | A | D | C | C |
| 18Z | A | D | D | D |
| 18AA | A | D | C | C |
| 18BB | A | D | D | D |
| 18CC | A | D | D | D |
| 18DD | A | D | D | D |
| 18EE | A | D | D | D |
| 18FF | B | D | D | D |
| 18GG | B | D | D | D |
| 18HH | A | D | D | D |
| 18II | B | D | D | D |
| 18JJ | B | D | D | D |
| 18KK | B | D | D | D |
| 18LL | B | D | D | D |
| 18MM | A | D | D | D |
| 18NN | A | D | D | D |
| 1800 | A | D | D | D |
| 18PP | B | D | D | D |
| 18QQ | B | D | D | D |
| 18RR | B | D | D | D |
| 18SS | B | D | D | D |
| 18TT | A | D | D | D |
| 18UU | B | D | D | D |
| 18VV | A | D | D | D |
| 18WW | A | D | D | D |
| 18XX | B | D | D | D |
| 18YY | B | D | D | D |
| 18ZZ | B | D | D | D |
| 18AAA | B | D | D | D |
| 18BBB | B | D | D | D |
| 18CCC | D | D | D | D |
| 18DDD | B | D | D | D |
| 18EEE | A | D | D | D |
| 18FFF | A | D | D | D |
| 18GGG | B | D | D | D |
| 18HHH | B | D | D | D |
| 18III | B | D | D | D |
| 18JJJ | B | D | D | D |
| 18KKK | B | D | D | D |
| 18LLL | B | D | D | D |
| 18MMM | B | D | D | D |
| 18NNN | B | D | D | D |
| 18000 | B | D | D | D |
| 18PPP | B | D | D | D |
| 18QQQ | B | D | D | D |
| 18RRR | B | D | D | D |
| 18SSS | B | D | D | D |
| 18TTT | B | D | D | D |
| 18UUU | A | D | D | D |
| 18VVV | B | D | D | C |
| 18WWW | A | D | D | D |
| 18XXX | B | D | D | D |
| 18YYY | A | D | D | C |
| 18ZZZ | A | D | C | C |
| 18AAAA | A | D | C | C |
| 18BBBB | B | D | D | D |
| 18CCCC | C | D | D | D |
| 18DDDD | B | D | D | D |
| 19A | B | D | D | D |
| 19B | C | D | D | D |
| 19C | B | D | D | D |
| 19D | B | D | D | D |
| 19E | B | D | D | D |
| 20A | A | D | D | D |
| 21A | B | D | D | D |
| 21B | B | D | D | D |
| 21C | B | D | D | D |
| 21D | B | D | D | D |
| 21E | A | D | D | D |
| 21F | B | D | D | D |
| 21G | A | D | C | C |
| 21H | C | D | D | D |
| 21I | A | D | C | C |
| 21J | B | D | D | D |
| 21K | B | D | D | D |
| 21L | B | D | D | D |
| 21M | B | D | D | C |
| 21N | B | D | D | D |
| 210 | B | D | D | D |
| 21P | B | D | D | D |
| 21Q | C | D | D | C |
| 21R | B | D | D | C |
| 21S | B | D | D | D |
| 21T | B | D | D | D |
| 21U | C | D | C | C |
| 21V | C | D | D | D |
| 21W | B | D | D | D |
| 21X | C | D | D | D |
| 21Y | B | D | C | C |
| 21Z | B | D | D | C |
| 21AA | B | D | D | C |
| 21BB | A | D | D | D |
| 21CC | B | D | D | D |
| 21DD | B | D | D | D |
| 21EE | A | D | D | D |
| 21FF | A | D | D | D |
| 21GG | B | D | D | D |
| 22A | B | D | D | D |
| 22B | C | D | D | D |
| 22C | D | D | D | D |
| 22D | B | D | D | D |
| 22E | D | D | D | D |
| 22F | D | D | D | D |
| 22G | D | D | D | D |
| 22H | D | D | D | D |
| 22I | D | D | D | D |
| 22J | D | D | D | D |
| 22K | D | D | D | D |
| 22L | D | D | D | D |
| 22M | C | D | D | D |
| 22N | C | D | D | D |
| 23A | B | D | D | D |
| 23B | B | D | D | D |
| 23C | C | D | D | D |
| 23D | C | D | D | D |
| 23E | C | D | D | D |
| 23F | C | D | D | D |
| 23G | D | D | D | D |
| 23H | C | D | D | D |
| 24A | A | D | C | C |
| 25A | B | D | D | D |
| 25B | D | D | D | D |
| 25C | D | D | D | D |
| 25D | C | D | D | D |
| 25E | C | D | D | D |
| 25F | C | D | D | D |
| 26A | B | D | D | D |

### Example 29

### Caco-2 Permeabitlity Assay

Cell membrane permeability of compounds of the present invention is determinted with the Caco-2 Permeability Assay.

### Preparation of Caco-2 Cells

Cell culture medium (25 mL) was added to a Transwell reservoir. Cell culture medium (50 µL) was added to each well of a 96-well HTS transwell plate and the plate then incubated at 37° C. and 5% CO₂ for 1 hour, before cell seeding. Caco-2 cells were diluted with culture medium, to 6.86×10⁵ cells/mL and 50 µL of cell suspension were dispensed into the filter well of the plate. Cells were cultivated for 14-18 days in a cell culture incubator at 37° C., 5% CO₂, 95% relative humidity. Cell culture medium was replaced every other day, beginning no later than 24 hours after initial plating.

### Assessment of Cell Monolayer Integrity

Medium was removed from the reservoir and from each well, and replaced with prewarmed fresh culture medium. Transepithelial electrical resistance (TEER) across the monolayer was measured using Millicell Epithelial Volt-Ohm measuring system (Millipore, USA) and the plate returned to the incubator once the measurement was complete. The TEER value was calculated according to the following equation:

$$\begin{matrix}\text{TEER measurement (ohms)} \times {\text{Area of membrane (cm}}^{2} ) \text{=} \\ \text{TEER value (ohm} \cdot {\text{cm}}^{2} ) \text{A TEER value greater than 230 ohm} \cdot {\text{cm}}^{2} \\ \text{indicates a suitable Caco-2 monolayer} \text{.}\end{matrix}$$

### Preparation of Solutions

### HBSS (25 mM HEPES, pH7.4)

HEPES (5.958 g) and sodium hydrogen carbonate (0.35 g) were added to pure water (900 mL), using sonication to dissolve solids, if required. HBSS (10x, 100 mL) was added to the solution, which was then placed on a stirrer. The pH was slowly adjusted to 7.4, by addition of sodium hydrate. The final solution was filtered before use.

### Compound Working Solution (5 µM)

A solution of compound — test or control (metoprolol, erythromycin or cimetidine) — (10 mM) was prepared and 6 µL was added to DMSO (54 µL) in the same well to obtain 1 mM stock solutions. Transport buffer (597 µL) was loaded into each well of a 96 well plate. 3 µL of 2 mM solution was added to each well to prepare the compound working solution.

Plates were shaken at 1000 rpm for 10 min.

### Drug Transport Assay

The apical to basolateral and basolateral to apical direction assays were performed simultaneously. The Caco-2 plates were removed from the incubator, the monolayer washed twice with pre-warmed HBSS (25 mM HEPES, pH 7.4) and then incubated at 37° C. for 30 minutes.

### Rate of Drug Transport - Apical to Basolateral Direction (A → B)

Working solution (108 µL) was added to the Transwell insert (apical compartment), and transfer 8 µL sample immediately from the apical compartment to 72 µL transport buffer and 240 µL of acetonitrile containing IS (100 nM alprazolam, 200 nM Caffeine and 100 nM tolbutamide) in a new 96-well plate as the initial donor sample (A-B). The plates were vortexed at 1000 rpm for 10 minutes. The wells in the receiver plate (basolateral compartment) were filled with transport buffer (300 µL).

### Rate of Drug Transport - Basolateral to Apical Direction (B → A)

Working solution (308 µL) was added to the receiver plate wells (basolateral compartment), and transfer 8 µL sample immediately from the basolateral compartment to 72 µL transport buffer and 240 µL of acetonitrile containing IS (100 nM alprazolam, 200 nM Caffeine and 100 nM tolbutamide) in a new 96-well plate as the initial donor sample (B-A). The plates were vortexed at 1000 rpm for 10 minutes. The Transwell insert (apical compartment) was filled with transport buffer (100 µL).

The multiwell insert plate was placed into the basolateral receiver plate, and incubated at 37° C. for 2 hours.

A sample from the donor side (8 µL, apical compartment for Ap→B1 flux, and basolateral compartment for B1→Ap flux) was transferred to a mixture of transport buffer (72 µL) and quenching solvent (240 µL) in new 96-well plates.

A sample from the receiver side (80 µL, basolateral compartment for Ap→B1 flux, and apical compartment for B1→Ap flux) was transferred to a mixture of acetonitrile (240 µL) and IS (100 nM alprazolam, 200 nM caffeine and 100 nM tolbutamide) in new 96-well plates.

The plates were vortexed for 10 minutes at 1000 rpm and then centrifuged at 4,000 rpm for 30 minutes. 100 µL of the supernatant was transferred to a new 96-well plate, taking care not to disturb the pellet. Pure water (100 µL) was added to all samples for analysis by LC-MS/MS. All incubations were performed in duplicates.

Lucifer yellow working solutions were prepared by diluting the stock solution with HBSS (25 mM HEPES, pH7.4) to a final concentration of 100 µM. 100 µL of the Lucifer yellow solution was added to the Transwell insert (apical compartment). The wells in the receiver plate (basolateral compartment) were filled with HBSS (300 µL, 25 mM HEPES, pH 7.4) and incubated at 37° C. for 30 minutes. An 80 µL aliquot was removed directly from the basolateral wells and transferred to new 96 wells plates. Measure Lucifer Yellow fluorescence (to monitor monolayer integrity) in a fluorescence plate reader at 485 nM excitation and 530 nM emission.

### Data Analysis

All calculations were carried out using Microsoft Excel. Peak areas are determined from extracted ion chromatograms.

### Lucifer Yellow Leakage

Lucifer yellow leakage of monolayer was calculated according to the following equation:

$$L Y L e a k a g e = \left(\frac{{I}_{a c c e p t o r} \times 0.3}{{I}_{a c c e p t o r} \times 0.3 + {I}_{d o n o r} \times 0.1}\right) \times 100 \%$$

I_{acceptor} is the fluorescence intensity in the acceptor well (0.3 mL)
I_{donor} is the fluorescence intensity in the donor well (0.1 mL)
Lucifer yellow (LY) leakage percentage amount transported values should be less than 1.5%.

### Apparent Permeability (Papp)

Apparent permeability (Papp) can be calculated for drug transport assays using the following equation

$${\text{P}}_{\text{app}} \text{=} \frac{\text{dQ} / \text{dt}}{{\text{A × D}}_{\text{0}}}$$

P ₐₚₚ is apparent permeability (cm/s × 10⁻⁶)
dQ/dt is the rate of drug transport (pmol/second)
A is the surface area of the membrane (cm²)
D₀ is the initial donor concentration (nM; pmol/cm³)

### Efflux Ratio

Efflux ratio can be determined using the following equation:

$$E f f l u x R a t i o = \frac{{P}_{a p p \left(B - A\right)}}{{P}_{a p p \left(A - B\right)}}$$

P ₐₚₚ_{(B-A)} is the apparent permeability coefficient for the basolateral to apical direction
P ₐₚₚ_{(A-B)} is the apparent permeability coefficient for the apical to basolateral direction

### Mass Balance

Mass balance (% recovery) can be determined using the following equation

$$\% r e c o v e r y = \frac{{\left[d r u g\right]}_{a c c e p t o r} \times 0.1 + {\left[d r u g\right]}_{d o n o r} \times 0.3}{{\left[d r u g\right]}_{i n i t i a l , d o n o r} \times 0.3} \times 100 \%$$

### Materials

Test compounds were prepared as described herein.

Caco-2 cells were obtained from the American type culture collection (ATCC, Number HTB-37).

Hepes, Penicillin, Streptomycin, Trypsin/EDTA and DMSO were purchased from Solarbio. Fetal bovine serum, Hank’s balanced salt solution (HBSS) and Non-essential amino acids (NEAA) were purchased from Gibco by Thermo Fisher Scientific. Dulbecco’s Modified Eagle’s Medium (DMEM) was purchased from Corning Corporation. HTS Transwell-96 Well (Cat. No. 3391) Permeable Supports were purchased from Corning Corporation. Millicell Epithelial Volt-Ohm measuring system was purchased from Millipore. Cellometer® Vision was purchased from Nexcelom Bioscience LLC. Infinite 200 PRO microplate reader was purchased from Tecan. MTS2/4 orbital shaker was purchased from IKA Labortechnik.

The various embodiments described above can be combined to provide further embodiments. All of the U.S. Pat., U.S. Pat, Application Publications, U.S. Pat. Applications, foreign patents, foreign patent applications and non-patent publications referred to in this specification and/or listed in the Application Data Sheet are incorporated herein by reference, in their entirety, to the same extent as if each individual publication, patent, or patent application was specifically and individually indicated to be incorporated by reference. Aspects of the embodiments can be modified, if necessary to employ concepts of the various patents, applications and publications to provide yet further embodiments.

This application claims the benefit of priority to U.S. Provisional Application No. 63/009,943, filed Apr. 14, 2020, which application is hereby incorporated by reference in its entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The present invention may be embodied in other specific forms without departing from the spirit or essential attributes thereof. This invention encompasses all combinations of preferred aspects and/or embodiments of the invention noted herein. It is understood that any and all embodiments of the present invention may be taken in conjunction with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

What is claimed is:

1. A compound having the structure of formula (II): 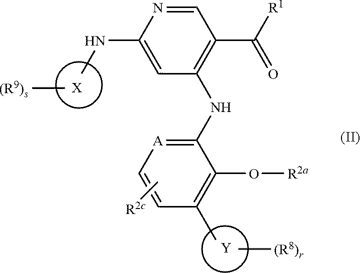
or a pharmaceutically acceptable isomer, racemate, hydrate, solvate, or salt thereof,
wherein:
A is N or CR^{2c};
ring X is a 5- or 6-membered heteroaryl;
ring Y is heteroaryl;
R¹ is C₁₋₄ alkyl, C₃₋₆ cycloalkyl, C₁₋₄ haloalkyl, C₁₋₄ hydroxyalkyl or alkoxyalkyl;
R^{2a} is H, C₁₋₄ alkyl or C₁₋₄ fluoroalkyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, C₁₋₆ haloalkyl, —OR^{11a}, —NR^{11a}R^{11b}, —SO₂R^{11a}, -SO₂NR^{11a}R^{11b}, —SO(═NH)R^{11a}, —C(O)R^{11a}, carbocycle, heterocycle, or (═O), wherein R¹⁰ is substituted with 0-2 R″;
wherein
R^{11a} is, at each occurrence, independently, H, halo, C₁₋₆ alkyl, C₁₋₆ haloalkyl, carbocycle, heterocycle, —CH₂CN, —OH, or —C(O)OH; and
R^{11b} is, at each occurrence, independently, H, halo, C₁₋₆ alkyl, C₁₋₆ haloalkyl, carbocycle, heterocycle, —CH₂CN, —OH, or —C(O)OH; or
R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle;
q is 0-4;
r is 0-2; and
s is 0-2.

2. The compound of claim 1, having the structure of Formula (III): 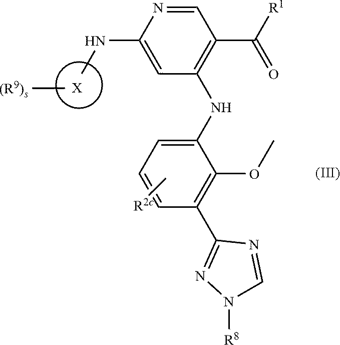
or a pharmaceutically acceptable salt, solvate, hydrate, isomer, tautomer, racemate, or isotope thereof, wherein:
ring X is a 5- or 6-membered heteroaryl;
R¹ is ethyl or cyclopropyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})_{q}—R¹⁰, —O—(CR^{a}R^{b})_{q}—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, C₁₋₆ haloalkyl, —OR^{11a}, —NR^{11a}R^{11b}, —SO₂R^{11a}, -SO₂NR^{11a}R^{11b}, —SO(═NH)R^{11a}, —C(O)R^{11a}, carbocycle, heterocycle, or (═O), wherein R¹⁰ is substituted with 0-2 R″; wherein
R^{11a} is, at each occurrence, independently, H, halo, C₁₋₆ alkyl, C₁₋₆ haloalkyl, carbocycle, heterocycle, —CH₂CN, —OH, or —C(O)OH; and
R^{11b} is, at each occurrence, independently, H, halo, C₁₋₆ alkyl, C₁₋₆ haloalkyl, carbocycle, heterocycle, —CH₂CN, —OH, or —C(O)OH; or
R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle;
q is 0-4;
r is 0-2; and
s is 0-2.

3. The compound of claim 1 or 2, or a pharmaceutically acceptable salt, solvate, hydrate, isomer, tautomer, racemate, or isotope thereof, wherein ring X is pyrrolyl, pyrazolyl, imidazolyl, triazolyl, furanyl, oxazolyl, oxadiazolyl, thiophenyl, thiazolyl, or thiadiazolyl.

4. The compound of any one of claims 1-3, having the structure of Formula (IV): 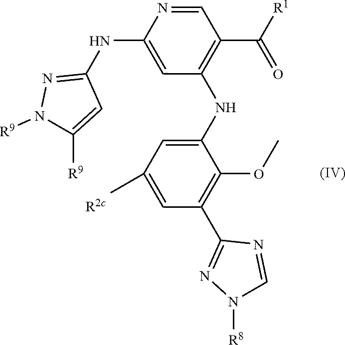
or a pharmaceutically acceptable salt, solvate, hydrate, isomer, tautomer, racemate, or isotope thereof, wherein:
R¹ is ethyl or cyclopropyl;
R^{Z°} is H, halo, —CN, C₁₋₄ alkyl, C₁₋₄ alkoxy, or C₁₋₄haloalkyl;
R⁸ is C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})q—R¹⁰, —O—(CR^{a}R^{b})q—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, C₁₋₆ haloalkyl, —OR^{11a}, —NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
R^{11a} is, at each occurrence, independently, H, halo, C₁₋₆ alkyl, C₁₋₆ haloalkyl, carbocycle, heterocycle, —CH₂CN, —OH, or —C(O)OH; and
R^{11b} is, at each occurrence, independently, H, halo, C₁₋₆ alkyl, C₁₋₆ haloalkyl, carbocycle, heterocycle, —CH₂CN, —OH, or —C(O)OH; or
R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4.

5. The compound of any one of claims 1-4, or a pharmaceutically acceptable salt, solvate, hydrate, isomer, tautomer, racemate, or isotope thereof, wherein R^{2c} is H.

6. The compound of any one of claims 1-4, or a pharmaceutically acceptable salt, solvate, hydrate, isomer, tautomer, racemate, or isotope thereof, wherein R^{2c} is halo, —CN, C₁₋₄ alkyl, C₁₋₄ haloalkyl, or C₁₋₄ alkoxy.

7. A compound having the structure of Formula (V): 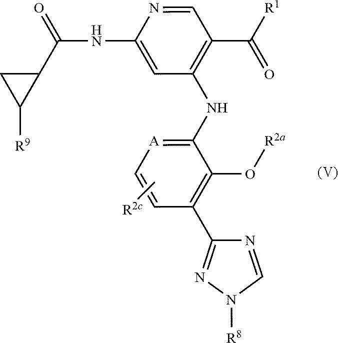
or a pharmaceutically acceptable salt, solvate, hydrate, isomer, tautomer, racemate, or isotope thereof, wherein:
A is N or CR^{2c.},
R¹ is C₁₋₄ alkyl, C₃₋₆ cycloalkyl, C₁₋₄ haloalkyl, C₁₋₄ hydroxyalkyl or alkoxyalkyl;
R^{2a} is H, C₁₋₄ alkyl or C₁₋₄ fluoroalkyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})q—R¹⁰, —O—(CR^{a}R^{b})q—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, C₁₋₆ haloalkyl, —OR^{11a}, —NR^{11a}R^{11b}, —SO₂R^{11a}, ―SO₂NR^{11a}R^{11b}, —SO(═NH)R^{11a}, —C(O)R^{11a}, carbocycle, heterocycle, or (═O), wherein R¹⁰ is substituted with 0-2 R″; wherein
R^{11a} is, at each occurrence, independently, H, halo, C₁₋₆ alkyl, C₁₋₆ haloalkyl, carbocycle, heterocycle, —CH₂CN, —OH, or —C(O)OH; and
R^{11b} is, at each occurrence, independently, H, halo, C₁₋₆ alkyl, C₁₋₆ haloalkyl, carbocycle, heterocycle, —CH₂CN, —OH, or —C(O)OH; or
R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4;
wherein R^{2c} is halo, —CN, C₁₋₄ alkyl, C₁₋₄ alkoxy, or C₁₋₄haloalkyl when R⁹ is H.

8. The compound of claim 7, having the structure of Formula (VI): 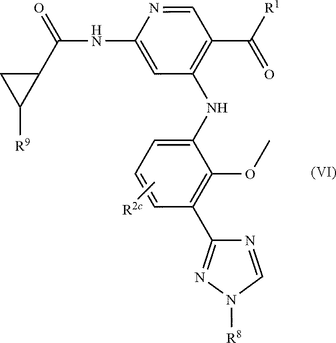
or a pharmaceutically acceptable salt, solvate, hydrate, isomer, tautomer, racemate, or isotope thereof, wherein:
R¹ is C₁₋₄ alkyl or C₃₋₆ cycloalkyl;
R^{2c} is H, halo, —CN, C₁₋₄ alkyl, C₁₋₄ alkoxy, or C₁₋₄haloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, H, halo, —CN, C₁₋₆ alkyl, C₁₋₆ haloalkyl, carbocycle, heterocycle, —(CR^{a}R^{b})q—R¹⁰, —O—(CR^{a}R^{b})q—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, C₁₋₆ haloalkyl, —OR^{11a}, —NR^{11a}R^{11b}, —SO₂R^{11a}, —SO₂NR^{11a}R^{11b}, —SO(═NH)R^{11a}, —C(O)R^{11a}, carbocycle, heterocycle, or (═O), wherein R¹⁰ is substituted with 0-2 R″; wherein
R^{11a} is, at each occurrence, independently, H, halo, C₁₋₆ alkyl, C₁₋₆ haloalkyl, carbocycle, heterocycle, —CH₂CN, —OH, or —C(O)OH; and
R^{11b} is, at each occurrence, independently, H, halo, C₁₋₆ alkyl, C₁₋₆ haloalkyl, carbocycle, heterocycle, —CH₂CN, —OH, or —C(O)OH; or
R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4;
wherein R^{2c} is halo, —CN, C₁₋₄ alkyl, C₁₋₄ alkoxy, or C₁₋₄haloalkyl when R⁹ is H.

9. The compound of claim 7 or 8, having the structure of Formula (VI-A): 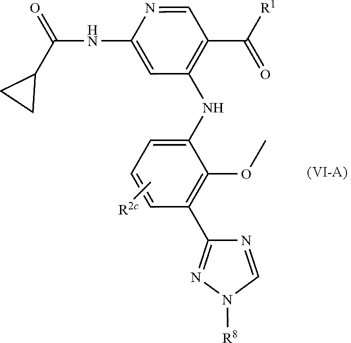
or a pharmaceutically acceptable salt, solvate, hydrate, isomer, tautomer, racemate, or isotope thereof, wherein:
R¹ is C₁₋₄ alkyl or C₃₋₆ cycloalkyl;
R^{2c} is halo, —CN, C₁₋₄ alkyl, C₁₋₄ alkoxy, or C₁₋₄haloalkyl; and
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle.

10. The compound of claim 7 or 8, having the structre of Formula (VI-B): 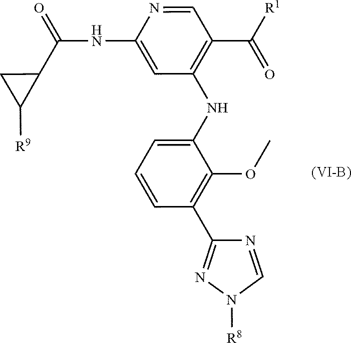
or a pharmaceutically acceptable salt, solvate, hydrate, isomer, tautomer, racemate, or isotope thereof, wherein:
R¹ is C₁₋₄ alkyl or C₃₋₆ cycloalkyl;
R⁸ is, at each occurrence, independently, C₁₋₆ alkyl, C₁₋₆ haloalkyl, C₁₋₆ hydroxyalkyl, C₁₋₆ alkoxy, C₁₋₆ haloalkoxy, alkoxyalkyl, or carbocycle;
R⁹ is, at each occurrence, independently, halo, —CN, C₁₋₆ alkyl, C₁₋₆ haloalkyl, carbocycle, heterocycle, —(CR^{a}R^{b})q—R¹⁰, —O—(CR^{a}R^{b})q—R¹⁰, —NR^{a}C(O)—R¹⁰, —C(O)—R¹⁰, or (═O), wherein R⁹ is substituted with 0-2 R″;
R^{a} is, at each occurrence, independently, H, C₁₋₆ alkyl, or C₁₋₆ haloalkyl;
R^{b} is, at each occurrence, independently, H, C₁₋₆ alkyl, or C₁₋₆ haloalkyl;
R¹⁰ is, at each occurrence, independently, H, halo, —CN, C₁₋₆ alkyl, C₁₋₆ haloalkyl, —OR^{11a}, —NR^{11a}R^{11b}, —SO₂R^{11a}, ―SO₂NR^{11a}R^{11b}, —SO(═NH)R^{11a}, —C(O)R^{11a}, carbocycle, heterocycle, or (═O), wherein R¹⁰ is substituted with 0-2 R″; wherein
R^{11a} is, at each occurrence, independently, H, halo, C₁₋₆ alkyl, C₁₋₆ haloalkyl, carbocycle, heterocycle, —CH₂CN, —OH, or —C(O)OH; and
R^{11b} is, at each occurrence, independently, H, halo, C₁₋₆ alkyl, C₁₋₆ haloalkyl, carbocycle, heterocycle, —CH₂CN, —OH, or —C(O)OH; or
R^{11a} and R^{11b}, together with the N atom to which they are attached, form an optionally substituted 4-, 5-, or 6-membered ring;
R″ is, at each occurrence, independently, H, C₁₋₄ alkyl, carbocycle, or heterocycle; and
q is 0-4.

11. The compound of any one of claims 1-10, or a pharmaceutically acceptable salt, solvate, hydrate, isomer, tautomer, racemate, or isotope thereof, wherein R¹ is ethyl.

12. The compound of any one of claims 1-10, or a pharmaceutically acceptable salt, solvate, hydrate, isomer, tautomer, racemate, or isotope thereof, wherein R¹ is cyclopropyl.

13. A compound of claim 1, wherein R¹ is C₁₋₄ alkyl, C₃₋₆ cycloalkyl, C₁₋₄ haloalkyl.

14. A compound having a structure listed in Table 1, or a pharmaceutically acceptable isomer, racemate, hydrate, solvate, isotope, or salt thereof.

15. A pharmaceutical composition comprising a compound of any one of claims 1-14, or a stereoisomer, tautomer, solvate or prodrug thereof, or a pharmaceutically acceptable salt thereof.

16. The composition of claim 15, further comprising a pharmaceutically acceptable carrier, adjuvant or vehicle.

17. A method of treating a disease responsive to the inhibition of TYK2 kinase activity in a patient, comprising administering to the patient a therapeutically effective amount of a composition of any one of claims 1-16.

18. The method of claim 17, wherein the disease is an inflammatory disease.

19. The method of claim 17, wherein the disease is asthma, inflammatory bowel disease, Crohn’s disease, ulcerative colitis, rheumatoid arthritis, psoriasis, allergic rhinitis, atopic dermatitis, contact dermatitis, delayed hypersensitivity reactions, lupus or multiple sclerosis.

20. The method of claim 17, further comprising administering a second therapeutic agent.

21. A kit comprising a pharmaceutical composition of claim 15 and instructions for use.

22. A compound according to any one of claims 1-14 for use in treating an inflammatory disease, in particular wherein the inflammatory disease is asthma, inflammatory bowel disease, Crohn’s disease, ulcerative colitis, rheumatoid arthritis, psoriasis, allergic rhinitis, atopic dermatitis, contact dermatitis, delayed hypersensitivity reactions, lupus or multiple sclerosis.

23. The use of a compound according to any one of claims 1-14 in the manufacture of a medicament for the treatment of an inflammatory disease, in particular wherein the inflammatory disease is asthma, inflammatory bowel disease, Crohn’s disease, ulcerative colitis, rheumatoid arthritis, psoriasis, allergic rhinitis, atopic dermatitis, contact dermatitis, delayed hypersensitivity reactions, lupus or multiple sclerosis.
